(19) United States
(12) Patent Application Publication
(10) Pub. No.: US 2024/0368283 A1
(43) Pub. Date: Nov. 7, 2024

(54) MOLECULES FOR MODULATING THE IMMUNE SYSTEM AND USES THEREOF

(71) Applicant: Seismic Therapeutic, Inc., Watertown, MA (US)

(72) Inventors: Nathan Higginson-Scott, Hingham, MA (US); Daniela Cipolletta, Belmont, MA (US); Jyothsna Visweswaraiah, Arlington, MA (US); Rebecca Goydel, Watertown, MA (US); Andre Stanlie, Watertown, MA (US); Kevin Lewis Otipoby, Ashland, MA (US); Stephen R. Lutz, Watertown, MA (US); Michael P. Cianci, Watertown, MA (US); Yen-Lin Chen, Cambridge, MA (US); Ryan Peckner, Berkeley, CA (US); Yanfeng Zhou, Boxborough, MA (US)

(21) Appl. No.: 18/648,099

(22) Filed: Apr. 26, 2024

Related U.S. Application Data
(60) Provisional application No. 63/609,741, filed on Dec. 13, 2023.
(60) Provisional application No. 63/601,426, filed on Nov. 21, 2023.
(60) Provisional application No. 63/462,096, filed on Apr. 26, 2023.

(51) Int. Cl. C07K 16/28

(52) U.S. Cl. CPC C07K 16/2818; C07K 16/283; C07K 2317/52; C07K 2317/622; C07K 2317/71; C07K 2317/75; C07K 2317/92

(57) ABSTRACT

Embodiments provided herein, provide for polypeptides, pharmaceutical compositions, and methods that can be used, for example, to target at least two types of cells to modulate the activity of the same to treat disorders, such as autoimmune disorders or cancers.

### CROSS-REFERENCE TO RELATED APPLICATIONS

This application claims priority to U.S. Provisional Application No. 63/462,096, filed Apr. 26, 2023, U.S. Provisional Application No. 63/601,426, filed Nov. 21, 2023, and U.S. Provisional Application No. 63/609,741, filed Dec. 13, 2023, each of which is hereby incorporated by reference in its entirety.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Apr. 23, 2024, is named “SES-009WO_SEQ.xml” and is 697,658 bytes in size.

### FIELD

The embodiments provided herein relate to compositions that target different cells to regulate an immune response.

### BACKGROUND

Cell-mediated immunity plays a critical role in the body's immune response. Unfortunately, uncontrolled cell-mediated immunity may lead to disease or auto-immune conditions. Most treatments available today regulate the body's immune response by targeting one factor. However, these treatments are not always effective, and, therefore, there is still a need for treatments that regulate cell-mediated immunity. In contrast, in treating cancers, there is a need to activate the body's immune response to target the cancer cells. The molecules immuno-oncology products approved today generally only target one type of cell through the binding of a single receptor, which can lead to an incomplete activation of an immune response to treat such cancers. The embodiments provided for herein fulfill these needs as well as others.

### SUMMARY

In some embodiments, an anti-PD-1 antibody, or an antigen-binding fragment thereof, comprising a polypeptide as provided for herein is provided.

In some embodiments, an anti-FcγRIIb antibody, or an antigen-binding fragment thereof, comprising a polypeptide as provided for herein is provided.

In some embodiments, polypeptides are provided comprising i) an anti-PD-1 antibody, or an antigen-binding fragment thereof; ii) an Fc polypeptide; and iii) an anti-FcγRIIb antibody, or antigen-binding fragment thereof, wherein the Fc polypeptide selectively binds to FcγRIIb or is effectorless.

In some embodiments, polypeptides are provided comprising i) an anti-PD-1 antibody, or an antigen-binding fragment thereof and an Fc polypeptide, wherein the Fc polypeptide selectively binds to FcγRIIb. In some embodiments, the polypeptide comprising the an anti-PD-1 antibody, or an antigen-binding fragment thereof and an Fc polypeptide, wherein the Fc polypeptide selectively binds to FcγRIIb does not comprise an antibody that selectively binds to FcγRIIb.

In some embodiments, methods of treating an autoimmune disorder in a subject are provided, the methods comprising administering to the subject a polypeptide, molecule or composition as provided for herein.

In some embodiments, methods of treating cancer in a subject are provided, the methods comprising administering to the subject a polypeptide, molecule or composition as provided for herein.

In some embodiments, methods of modulating two types of cells with a polypeptide, the methods comprising contacting the two types of cells, a polypeptide, molecule or composition as provided for herein.

In some embodiments, methods of modulating the activity of two types of cells in a subject are provided, the method comprising administering to the subject a polypeptide, molecule or composition as provided for herein.

In some embodiments, methods of inhibiting i) an activated immune cell (e.g. T-cell); and ii) the activity of a B-Cell, an antigen presenting cell (APC), or a myeloid cell, the methods comprising administering to a subject or contacting the activated immune cell and the B Cell or antigen presenting cell with a polypeptide, molecule or composition as provided for herein.

In some embodiments, methods of inhibiting or enhancing an activated immune cell (e.g. T-cell) and the activity of B-Cell, an antigen presenting cell (APC), or a myeloid cell are provided, the methods comprising administering to a subject or contacting the activated immune cell and the B Cell or antigen presenting cell with a polypeptide, molecule or composition as provided for herein.

In some embodiments, nucleic acid molecules encoding the polypeptides as provided for herein are provided.

### BRIEF DESCRIPTION OF THE DRAWINGS

FIG. 1 depicts a non-limiting illustration of how a therapeutic compound provided herein could function.

FIG. 2A depicts a non-limiting illustration of the therapeutic compounds provided herein.

FIG. 2B depicts a non-limiting illustration of the therapeutic compounds provided herein.

FIG. 3 depicts a non-limiting illustration of the therapeutic compounds provided herein.

FIG. 4 depicts a non-limiting illustration of the therapeutic compounds provided herein.

FIG. 5 depicts a non-limiting illustration of the therapeutic compounds provided herein.

FIG. 6 depicts a non-limiting illustration of the therapeutic compounds provided herein.

FIG. 7 depicts a non-limiting illustration of the therapeutic compounds provided herein.

FIG. 8 depicts a non-limiting illustration of the therapeutic compounds provided herein.

FIG. 9 depicts a non-limiting illustration of the therapeutic compounds provided herein.

FIG. 10 depicts a non-limiting illustration of the therapeutic compounds provided herein.

FIG. 11 depicts a non-limiting illustration of the therapeutic compounds provided herein.

FIG. 12 depicts a non-limiting illustration of the therapeutic compounds provided herein.

FIG. 13 depicts a non-limiting illustration of the therapeutic compounds provided herein.

FIG. 14 depicts a non-limiting illustration of the therapeutic compounds provided herein.

FIG. 15 illustrates binding affinities of various test articles.

FIG. 16 illustrates PD-1 agonism of various test articles.

FIG. 17 illustrates PD-1 agonism of various test articles in presence or absence of FcγRIIb.

FIG. 18A illustrates PD-1 agonism mediated by selective anchoring to FcγRIIb via the scFv moiety of various test articles.

FIG. 18B illustrates lacks of PD-1 agonism mediated by selective anchoring to FcγRIIb via the scFv moiety of various test articles in FcγRIIb deficient cells.

FIG. 19 illustrates TNF production as stimulated by various test articles.

FIG. 20 illustrates PD-1 agonism as induced by various test articles.

FIG. 21 illustrates PD-1 agonism as induced by various test articles.

FIG. 22 illustrates lack of PD-1 agonism as induced by various test articles.

FIG. 23 illustrates lack of PD-1 antagonism as induced by various test articles.

FIG. 24A illustrates binding affinities of various articles.

FIG. 24B illustrates binding affinities of various articles.

FIG. 24C illustrates binding affinities of various articles.

FIG. 25 illustrates TNF-alpha production in response to various articles.

FIG. 26 illustrates granzyme B and IFN-gamma production in response to various articles.

FIG. 27A illustrates ADCC induction in response to various articles.

FIG. 27B illustrates ADCC induction in response to various articles.

FIG. 28A illustrates IL-2 expression in response to various articles.

FIG. 28B illustrates IFN-gamma expression in response to various articles.

FIG. 28C illustrates TNF-alpha expression in response to various articles.

FIG. 29 illustrates C1q binding to various articles.

FIG. 30A illustrates ADCC induction in response to various articles.

FIG. 30B illustrates ADCC induction in response to various articles.

FIG. 31A illustrates PD-1 binding affinities of various articles.

FIG. 31B illustrates IL-2 expression in response to various articles.

FIG. 32 illustrates surface PD-1 loss in response to various articles.

FIG. 33A illustrates FcγRIIβ agonism data.

FIG. 33B illustrates FcγRIIβ agonism data.

### DETAILED DESCRIPTION

As used herein and unless otherwise indicated, the term “about” means that the numerical value is approximate and small variations would not significantly affect the practice of the disclosed embodiment. Where a numerical limitation is used, unless indicated otherwise by the context, “about” means the numerical value can vary by ±10% and remain within the scope of the disclosed embodiments.

As used herein and in the appended claims, the singular forms “a”, “an” and “the” include plural reference unless the context clearly dictates otherwise.

As used herein, the term “animal” includes, but is not limited to, humans and non-human vertebrates such as wild, domestic, and farm animals. Accordingly, as used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “contacting” means bringing together of two elements in an in vitro system or an in vivo system. For example, “contacting” a therapeutic compound with an individual or patient or cell includes the administration of the compound or composition to an individual or patient, such as a human, as well as, for example, introducing a compound into a sample containing a cellular or purified preparation containing target.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “fused” or “linked” when used in reference to a protein or molecule having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another.

As used herein, the term “individual,” “subject,” or “patient,” which can be used interchangeably, means any animal, including mammals, such as mice, rats, other rodents, rabbits, dogs, cats, swine, cattle, sheep, horses, or primates, such as humans.

As used herein, the term “inhibit” refers to a result, symptom, or activity being reduced as compared to the activity or result in the absence of the compound that is inhibiting the result, symptom, or activity. In some embodiments, the result, symptom, or activity, is inhibited by about, or, at least, 10%, 20%, 30%, 40%, 50%, 60%, 70%, 80%, 90%, 95%, or 99%. An result, symptom, or activity can also be inhibited if it is completely elimination or extinguished.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

As used herein, the phrase “ophthalmically acceptable” means having no persistent detrimental effect on the treated eye or the functioning thereof, or on the general health of the subject being treated. However, it will be recognized that transient effects such as minor irritation or a “stinging” sensation are common with topical ophthalmic administration of drugs and the existence of such transient effects is not inconsistent with the composition, formulation, or ingredient (e.g., excipient) in question being “ophthalmically acceptable” as herein defined. In some embodiments, the pharmaceutical compositions can be ophthalmically acceptable or suitable for ophthalmic administration.

As used herein, the term “position,” is meant to refer to a location in the sequence of a polypeptide. Positions may be numbered sequentially, or according to an established format, such as, but not limited to, the EU Index or numbering system based on Kabat's amino acid positions for antibodies or Fc domains.

In some embodiments, the term “therapeutic molecule” can be used interchangeably with “therapeutic compound,” “molecule,” or “therapeutic,” and refers to any polypeptide, or protein described herein.

“Specific binding” or “specifically binds to” or is “specific for” a particular antigen, target, or an epitope means binding that is measurably different from a non-specific interaction.

Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen, target, or an epitope can be exhibited, for example, by an antibody having a K_{D} for an antigen or epitope of at least about 10^{-4M}, at least about 10^{-5M}, at least about 10^{-6M}, at least about 10^{-7M}, at least about 10^{-8M}, at least about 10^{-9M}, alternatively at least about 10^{-10M}, at least about 10^{-11M}, at least about 10^{-12M}, or greater, where K_{D} refers to a dissociation rate of a particular antibody-target interaction. Typically, an antibody that specifically binds an antigen or target will have a K_{D} that is, or at least, 2-, 4-, 5-, 10-, 20-, 50-, 100-, 500-, 1000-, 5,000-, 10,000-, or more times greater for a control molecule relative to the antigen or epitope.

In some embodiments, specific binding for a particular antigen, target, or an epitope can be exhibited, for example, by an antibody having a K_{A} or Kₐ for a target, antigen, or epitope of at least 2-, 4-, 5-, 20-, 50-, 100-, 500-, 1000-, 5,000-, 10,000- or more times greater for the target, antigen, or epitope relative to a control, where K_{A} or Kₐ refers to an association rate of a particular antibody-antigen interaction.

As provided herein, the compounds and compositions provided for herein can be used in methods of treatment as provided herein. As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. For purposes of these embodiments, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival, as applicable for a specific disease, as compared to expected survival if not receiving treatment. Thus, “treatment of an autoimmune condition” or “treating autoimmunity” means an activity that alleviates or ameliorates any of the primary phenomena or secondary symptoms associated with the autoimmune condition other condition described herein when the terms “treat,” “treated,” or “treating” are used in conjunction with such condition.

As used herein, terms “variant,” “molecule,” “therapeutic,” “therapeutic compound,” “compound,” “polypeptide,” or “protein” can be used interchangeably and relate to the variants, molecules, therapeutics, therapeutic compounds, compounds, polypeptides, and proteins disclosed herein.

Provided herein are compounds, such as antibodies, polypeptides or fusion proteins, e.g., that can be used as therapeutics that include two or more effector domains that bind to at least two different immune cell types. In some embodiments, the compound comprises 2, 3, or 4 effector domains, such as an inhibitory receptor effector domain. In some embodiments, the compounds binds to at least 2 different cell surface receptors molecules, with at least one being on two different cell types. In some embodiments, the compound can comprise 3 effector domains, wherein at least two of the effector domains, which can be inhibitory receptor effector domains, bind to different cell surface receptors, but the at least two of the effector domains bind to the different cell surface receptors on the same cell or cell type. For example, a polypeptide can comprise an inhibitory receptor effector domain that binds to PD-1 and a second inhibitory receptor effector domain that binds to LAG-3. The interaction of these domains with PD-1 and LAG-3 can be, for example on the same cell or it can be on the same cell type, but wherein the PD-1 and LAG-3 are on different cells. In some embodiments, the effector domains can modulate the activity of the cell that they bind to by modulating the activity of the cell surface receptor to which they bind to. In some embodiments, each effector domain, independently, agonizes the activity of molecule to which it binds to. In some embodiments, each effector domain, independently, antagonizes the activity of molecule to which it binds to.

Without being bound to any particular theory, the effector domains by binding to two different cells at the same time, nearly the same time, or in the same local environment, the compounds provided herein can modulate the cell-mediated immunity being regulated by those cells. In some embodiments, the immune response is suppressed. In some embodiments, the immune response is activated. When the immune response is suppressed, the polypeptide can be used to, for example, treat an auto-immune disease or condition, such as those provided for herein. When the immune response is activated, the polypeptide can be used to, for example, treat cancer or other proliferative disorder, such as those provided for herein.

Also provided are methods of using and making the compounds.

In some embodiments, a polypeptide is provided that comprises: a) an inhibitory receptor effector domain; b) a Fc domain; and c) a FcγRII binding effector domain. In some embodiments, a polypeptide is provided that comprises: a) an inhibitory receptor effector domain and b) a Fc domain. In some embodiments, a polypeptide is provided that comprises: a) an inhibitory receptor effector domain and b) a FcγRII binding effector domain. In some embodiments, a polypeptide is provided that comprises: a) an inhibitory receptor effector domain; b) a Fc domain; and c) a FcγRII binding effector domain. In some embodiments, a polypeptide is provided that comprises an inhibitory receptor effector domain and a FcγRII binding effector domain, i.e., without an Fc domain. In some embodiments, a polypeptide is provided that comprises a plurality of inhibitory receptor effector domains and a Fc domain linked to each inhibitory receptor effector domain. The Fc polypeptides linked to each inhibitory receptor effector domain can be the same or different. In some embodiments, a polypeptide is provided that comprises 1, 2, 3, or 4 inhibitory receptor effector domains, each linked to a Fc domain. The Fc polypeptides linked to each inhibitory receptor effector domain can be the same or different. In some embodiments, the inhibitory receptor domains are linked to the Fc polypeptide to the N-terminus and/or the C-terminus of the Fc polypeptide. In some embodiments, each Fc domain has 1 or 2 inhibitory receptor domains linked to the Fc polypeptide. In some embodiments, the Fc polypeptide has an inhibitory effector domain linked to the N-terminus and the C-terminus of the Fc polypeptide. In some embodiments, the inhibitory effector domains binds to the same inhibitory receptor. In some embodiments, the inhibitory effector domain binds to different inhibitory receptors.

In some embodiments, the polypeptide comprises from the N-terminus to the C-terminus: a) an inhibitory receptor effector domain; b) a Fc domain; and c) a FcγRII binding effector domain. In some embodiments, the polypeptide comprises from the N-terminus to the C-terminus a) a FcγRII binding effector domain b) a Fc domain; and c) an inhibitory receptor effector domain.

In some embodiments, the polypeptide comprises from the N-terminus to the C-terminus: an inhibitory receptor effector domain and a FcγRII binding effector domain. In some embodiments, the polypeptide comprises from the N-terminus to the C-terminus: a FcγRII binding effector domain and an inhibitory receptor effector domain.

In some embodiments, the polypeptide comprises from the N-terminus to the C-terminus: a) an inhibitory receptor effector domain and a Fc domain. In some embodiments, the polypeptide comprises from the N-terminus to the C-terminus a) Fc domain and an inhibitory receptor effector domain.

In each of the embodiments, provided for herein, the domains can be linked to one another with a peptide linker, such as the non-limiting examples provided for herein, or without an intervening peptide linker.

In some embodiments, the polypeptide comprises a plurality of inhibitory receptor effector domains that can bind to either the same inhibitory receptors or to two different inhibitory receptors. As provided for herein, in some embodiments, the polypeptide comprises two inhibitory receptor effector domains that bind to the same or different inhibitory receptors.

As used herein, the term “inhibitory receptor effector domain” refers to a polypeptide, such as an antibody, that binds to an inhibitory receptor present on an immune cell, such as, but not limited to, T-cells. In some embodiments, the T-cell is an activated T-cell. In some embodiments, the T-cell is not activated. In some embodiments, the polypeptide comprises one or more inhibitory receptor effector domains. In some embodiments, the polypeptide comprises 2, 3, or 4 inhibitory receptor effector domains. In some embodiments, the inhibitory receptor effector domains bind to the same inhibitory receptors. In some embodiments, the different inhibitory receptor effector domains bind to different inhibitory receptors. For example, if the polypeptide comprises two inhibitory receptor effector domains that bind to different inhibitory receptors, the first inhibitory receptor effector domain can bind to a first inhibitory receptor and the second inhibitory receptor effector domain can bind to a second inhibitory receptor that is different from the first. In some embodiments, the inhibitory receptor effector domain is an antibody. In some embodiments, the antibody is a Fab format antibody. In some embodiments, the antibody is a scFv antibody. In some embodiments, the antibody is an antibody as provided for herein. In some embodiments, the polypeptide comprises an inhibitory receptor effector domain that is an antibody in a Fab format and an inhibitory receptor effector domain that is an scFv antibody.

In some embodiments, the antibody binds to PD-1. In some embodiments, the polypeptide comprises an Fc domain that selectively binds to FcγRIIβ. In some embodiments, the molecule comprises an antibody that binds to PD-1 and comprises an Fc domain that selectively binds to FcγRIIβ. Examples of each of these types of molecules are provided for herein.

In some embodiments, the molecule provided for herein comprise an antibody that selectively binds to FcγRIIβ. In some embodiments, the antibody that selectively binds to FcγRIIβ comprises an Fc domain. The Fc domain may be an effectorless Fc domain, or may comprise mutations that allow the Fc domain to also selectively bind with FcγRIIβ. As used herein, in reference to an antibody that selectively binds to FcγRIIβ, the term “selectively binds to” means that the antibody preferentially binds to FcγRIIβ, that is with a higher affinity to FcγRIIβ as compared to other Fey receptors, such as FcγRIIα.

Additionally, in some embodiments, the molecule may comprise other domains that bind to other molecules or another molecule of interest. In some embodiments, a polypeptide comprises an inhibitory receptor effector domain that also binds to LAG3, PDCD1, BTLA/CD272, CD200R1, CD22/Siglec2, CD300A, CD300LF/CD300F, CD33/Siglec3, CD5, CD72, CEACAM 1, CLEC12A, CLEC4A, CTLA4/CD152, FCGR2B/CD32B, KIRs, KLRB1/CD161, KLRC1, KLRG1, LAIR1, LILRB1, LILRB2, LILRB4, LILRB5, NCR2/NKp44, PECAM1/CD31, PILRA, PVR/CD155, SIGLEC11, SIGLEC5, SIGLEC7, SIGLEC8, SIGLEC9, SIRPA, TIGIT, VSTM1/SIRL1, MAFA, NKG2A, CMRF35H, CD66a, CD66d, CD33, SIGLEC6, ILT2,3,4,5, LIR8, KIR2DL, KIR2DL1, KIR3DL, SIRPa, KIR2DL2/3, KIR2DL5, KIRDL1, KIRDL2, KIRDL3, TIM3, Tactile, IRp60, NKRP1, IAP, PIR-B, CD5, 2B4, GP49B, Ly49Q, MICL, CD160, FCRL4, KIR3DL1, KIR2DL2, LILRB3, DCIR, NKRP-1D, LY49, MAIR-I, CD79a, CD79b, CD19, CD21, CD40, TLR3, CD28, CCR5, or CCR1.

In some embodiments, the other molecule is LAG3.

In some embodiments, the other molecule is an antibody that binds to FcγRIIβ. Thus, the molecule may be a bispecific or trispecific for different proteins, such as PD-1, LAG3, and/or FcγRIIβ. Additionally, the molecule may comprise an Fc domain that specifically binds to FcγRIIβ, such as, but not limited to, those provided for herein.

In some embodiments, polypeptide comprises an inhibitory receptor effector domain that binds to PD-1 and a second inhibitory receptor that binds to LAG-3. In some embodiments, polypeptide comprises an inhibitory receptor effector domain that binds to LAG-3 and a second inhibitory receptor that binds to PD-1.

As used herein, “isotype” refers to the immunoglobulin class (e.g., IgG1, IgG2, IgG3, IgG4, IgM, IgA1, IgA2, IgD, and IgE antibody) that is encoded by the heavy chain constant domain genes. The full-length amino acid sequence of each wild type human IgG constant region (including all domains, i.e., CH1 domain, hinge, CH2 domain, and CH3 domain) is cataloged in the UniProt database available on-line, e.g., as P01857 (IgG1), P01859 (IgG2), P01860 (IgG3), and P01861 (IgG4), or different allotypes thereof (SEQ ID NOs: 1, 2, 3, and 4, respectively). As used herein, a domain of a heavy chain constant region, e.g., the hinge, is of an “IgG1 isotype,” “IgG2 isotype,” “IgG3 isotype,” or “IgG4 isotype,” if the domain comprises the amino acid sequence of the corresponding domain of the respective isotype, or a variant thereof (that has a higher homology to the corresponding domain of the respective isotype than it does to that of the other isotypes).

“Allotype” refers to naturally occurring variants within a specific isotype group, which variants differ in a few amino acids (see, e.g., Jefferies et al. (2009) mAbs 1:1). Molecules described herein may be of any allotype.

A “wild-type” protein or portion thereof is a version of the protein as it is found in nature. An amino acid sequence of a wild-type protein, e.g., a heavy chain constant region, is the amino acid sequence of the protein as it occurs in nature. Due to allotypic differences, there can be more than one amino acid sequence for a wild-type protein. For example, there are several allotypes of naturally occurring human IGg1 heavy chain constant regions (e.g., Jeffries et al. (2009) mAbs 1:1).

An immunoglobulin may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. In certain embodiments, the antibodies described herein are of the human IgG1 or IgG2 subtype. Immunoglobulins, e.g., human IgG1, exist in several allotypes, which differ from each other in at most a few amino acids.

In some embodiments, the IgG proteins (hinge region underlined) are as provided in Table 1.

TABLE 1

| Isotype | Sequence |
|---|---|
| IgG1 | ASTKGPSVFPLAPSSKSTSGGTAALGCLVKDYFPEPVTVSWNSGALTSGVHTFPAVLQSS |
| | GLYSLSSVVTVPSSSLGTQTYICNVNHKPSNTKVDKKVEPKSCDKTHTCPPCPAPELLGG |
| | PSVFLFPPKPKDTLMISRTPEVTCVVVDVSHEDPEVKFNWYVDGVEVHNAKTKPREEQYN |
| | STYRVVSVLTVLHQDWLNGKEYKCKVSNKALPAPIEKTISKAKGQPREPQVYTLPPSRDE |
| | LTKNQVSLTCLVKGFYPSDIAVEWESNGQPENNYKTTPPVLDSDGSFFLYSKLTVDKSRW |
| | QQGNVFSCSVMHEALHNHYTQKSLSLSPGK (SEQ ID NO: 516) |
| IgG2 | ASTKGPSVFPLAPCSRSTSESTAALGCLVKDYFPEPVTVSWNSGALTSGVHTFPAVLQSS |
| | GLYSLSSVVTVPSSNFGTQTYTCNVDHKPSNTKVDKTVERKCCVECPPCPAPPVAGPSVF |
| | LFPPKPKDTLMISRTPEVTCVVVDVSHEDPEVQFNWYVDGVEVHNAKTKPREEQFNSTFR |
| | VVSVLTVVHQDWLNGKEYKCKVSNKGLPAPIEKTISKTKGQPREPQVYTLPPSREEMTKN |
| | QVSLTCLVKGFYPSDISVEWESNGQPENNYKTTPPMLDSDGSFFLYSKLTVDKSRWQQGN |
| | VFSCSVMHEALHNHYTQKSLSLSPGK (SEQ ID NO: 517) |
| IgG3 | ASTKGPSVFPLAPCSRSTSGGTAALGCLVKDYFPEPVTVSWNSGALTSGVHTFPAVLQSS |
| | GLYSLSSVVTVPSSSLGTQTYTCNVNHKPSNTKVDKRVELKTPLGDTTHTCPRCPEPKSC |
| | DTPPPCPRCPEPKSCDTPPPCPRCPEPKSCDTPPPCPRCPAPELLGGPSVFLFPPKPKDT |
| | LMISRTPEVTCVVVDVSHEDPEVQFKWYVDGVEVHNAKTKPREEQYNSTFRVVSVLTVLH |
| | QDWLNGKEYKCKVSNKALPAPIEKTISKTKGQPREPQVYTLPPSREEMTKNQVSLTCLVK |
| | GFYPSDIAVEWESSGQPENNYNTTPPMLDSDGSFFLYSKLTVDKSRWQQGNIFSCSVMHE |
| | ALHNRFTQKSLSLSPGK (SEQ ID NO: 518) |
| IgG4 | ASTKGPSVFPLAPCSRSTSESTAALGCLVKDYFPEPVTVSWNSGALTSGVHTFPAVLQSS |
| | GLYSLSSVVTVPSSSLGTKTYTCNVDHKPSNTKVDKRVESKYGPPCPSCPAPEFLGGPSV |
| | FLFPPKPKDTLMISRTPEVTCVVVDVSQEDPEVQFNWYVDGVEVHNAKTKPREEQFNSTY |
| | RVVSVLTVLHQDWLNGKEYKCKVSNKGLPSSIEKTISKAKGQPREPQVYTLPPSQEEMTK |
| | NQVSLTCLVKGFYPSDIAVEWESNGQPENNYKTTPPVLDSDGSFFLYSRLTVDKSRWQEG |
| | NVFSCSVMHEALHNHYTQKSLSLSLGK (SEQ ID NO: 519) |

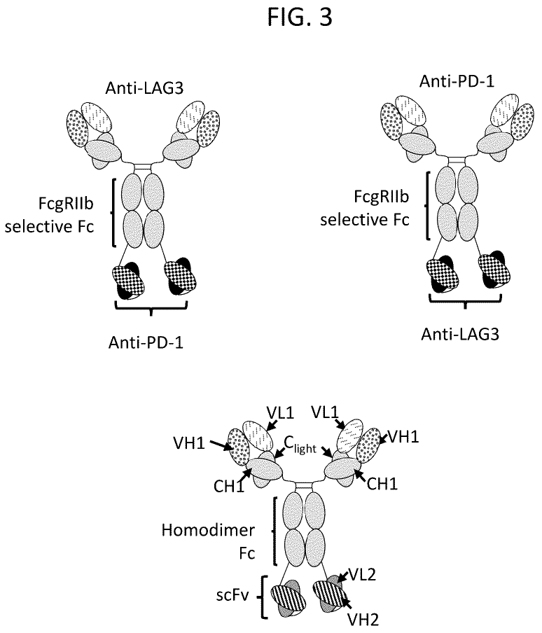

An “Fc polypeptide” (fragment crystallizable region), “Fc domain”, “Fc”, or “constant domain” or an antibody refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc polypeptide of an antibody of isotype IgG comprises the heavy chain constant region of the antibody excluding the first constant region immunoglobulin domain (CH1). In IgG, IgA and IgD antibody isotypes, the Fc polypeptide comprises CH2 and CH3 constant domains in each of the antibody's two heavy chains; IgM and IgE Fc polypeptides comprise three heavy chain constant domains (CH domains 2-4) in each polypeptide chain. For IgG, the Fc polypeptide comprises immunoglobulin domains consisting of the hinge, CH2 and CH3. For purposes herein, the Fc polypeptide is defined as starting at amino acid 216 and ending at amino acid 447, wherein the numbering is according to the EU index as in Kabat. Kabat et al. (1991) Sequences of Proteins of Immunological Interest, National Institutes of Health, Bethesda, MD, and according to FIGS. 3c-3f of U.S. Pat. App. Pub. No. 2008/0248028. The “EU index” may also be referred to as “EU numbering”. In some embodiments, the Fc polypeptide comprises the hinge region. The Fc may be a native (or naturally-occurring or wild-type) Fc, including any allotypic variant, or a variant Fc (e.g., a non-naturally occurring Fc), comprising, e.g., 1, 2, 3, 4, 5, 1-5, 1-10 or 5-10 or more amino acid mutations, e.g., substitutions, additions or deletions. For example, a variant Fc may comprise an amino acid sequence that is at least 75%, 80%, 85%, 90%, 95%, 96%, 97%, 98% or 99% identical to a wild-type Fc. Modified or mutated Fes may have enhanced or reduced effector function and/or half-life. Fc may refer to this region in isolation or in the context of an Fc-comprising protein polypeptide such as a “binding protein comprising an Fc polypeptide,” also referred to as an “Fc fusion protein” (e.g., an antibody or immunoadhesin). In some embodiments, modified or variant Fc molecules have enhanced binding to FcγRIIβ.

A “hinge”, “hinge domain” or “hinge region” or “antibody hinge region” refers to the domain of a heavy chain constant region that joins the CH1 domain to the CH2 domain and includes the upper, middle, and lower portions of the hinge (Roux et al. J. Immunol. 1998 161:4083). The hinge provides varying levels of flexibility between the binding and effector regions of an antibody and also provides sites for intermolecular disulfide bonding between the two heavy chain constant regions. The term “hinge” includes wild-type hinges (such as those set forth in Table 2), as well as variants thereof (e.g., non-naturally-occurring hinges or modified hinges). For example, the term “IgG1 hinge” includes wild-type IgG1 hinge, as shown below, and variants having 1, 2, 3, 4, 5, 1-3, 1-5, 3-5 and/or at most 5, 4, 3, 2, or 1 mutations, e.g., substitutions, deletions or additions. In some embodiments, the hinge regions are as provided in Table 2.

TABLE 2

| Isotype | Hinge Sequence |
|---|---|
| IgG1 | EPKSCDKTHTCPPCPAPELLGGP (SEQ ID NO: 520) |
| IgG2 | ELKTPLGDTTHTCPRCPAPELLGGP (SEQ ID NO: 521) |
| IgG3 | ELKTPLGDTTHTCPRCPEPKSCDTPPPCPRCPEPKSCDTPP |
| | PCPRCPEPKSCDTPPPCPRCPAPELLGGP |
| | (SEQ ID NO: 522) |
| IgG4 | ESKYGPPCPSCPAPEFLGGP (SEQ ID NO: 523) |

The term “CH1 domain” refers to the heavy chain constant region linking the variable domain to the hinge in a heavy chain constant domain. As used herein, a CH1 domain includes wild type CH1 domains, as well as variants thereof (e.g., non-naturally-occurring CH1 domains or modified CH1 domains). As used herein, CH1 domain includes amino acid residues 1-98 of IgG1; 1-98 of IgG2; 1-98 of IgG3; and 1-98 of IgG4. For example, the term “CH1 domain” includes wild-type CH1 domains and variants thereof having 1, 2, 3, 4, 5, 1-3, 1-5, 3-5 and/or at most 5, 4, 3, 2, or 1 mutations, e.g., substitutions, deletions or additions.

The term “CH2 domain” refers to the heavy chain constant region linking the hinge to the CH3 domain in a heavy chain constant domain. As used herein, a CH2 domain includes wild-type CH2 domains, as well as variants thereof (e.g., non-naturally-occurring CH2 domains or modified CH2 domains). As used herein, CH2 domain includes amino acid residues 111-223 of IgG1; 111-219 of IgG2; 161-270 of IgG3; and 111-220 of IgG4. For example, the term “CH2 domain” includes wild-type CH2 domains and variants thereof having 1, 2, 3, 4, 5, 1-3, 1-5, 3-5 and/or at most 5, 4, 3, 2, or 1 mutations, e.g., substitutions, deletions or additions.

The term “CH3 domain” refers to the heavy chain constant region that is C-terminal to the CH2 domain in a heavy chain constant domain. As used herein, a CH3 domain includes wild-type CH3 domains, as well as variants thereof (e.g., non-naturally-occurring CH3 domains or modified CH3 domains). As used herein, CH3 domain includes amino acid residues 224-330 of IgG1; 220-326 of IgG2; 271-376 of IgG3; and 226-322 of IgG4. For example, the term “CH3 domain” includes wild-type CH3 domains and variants thereof having 1, 2, 3, 4, 5, 1-3, 1-5, 3-5 and/or at most 5, 4, 3, 2, or 1 mutations, e.g., substitutions, deletions or additions.

In some embodiments, the hinge/CH2 domain has an amino acid sequence such as those provided in Table 3 below.

TABLE 3

| Isotype | Hinge/CH2 Sequence |
|---|---|
| IgG1 | PCPAPELLGGPSVFLFPPKPKDTLMISRTPEVTCVVVDVS |
| | HEDPEVKENWYVDGVEVHNAKTKPREEQYNSTYRVVSVLT |
| | VLHQDWLNGKEYKCKVSNKALPAPIEKTISKAK (SEQ |
| | ID NO: 524) |
| IgG2 | APPVAGPSVFLFPPKPKDTLMISRTPEVTCVVVDVSHEDP |
| | EVQFNWYVDGVEVHNAKTKPREEQFNSTFRVVSVLTVVHQ |
| | DWLNGKEYKCKVSNKGLPAPIEKTISKTK (SEQ ID |
| | NO: 525) |
| IgG3 | APELLGGPSVFLFPPKPKDTLMISRTPEVTCVVVDVSHED |
| | PEVQFKWYVDGVEVHNAKTKPREEQYNSTFRVVSVLTVLH |
| | QDWLNGKEYKCKVSNKALPAPIEKTISKTK (SEQ ID |
| | NO: 526) |
| IgG4 | APEFLGGPSVFLFPPKPKDTLMISRTPEVTCVVVDVSQED |
| | PEVQFNWYVDGVEVHNAKTKPREEQENSTYRVVSVLTVLH |
| | QDWLNGKEYKCKVSNKGLPSSIEKTISKAK (SEQ ID |
| | NO: 527) |

Without being bound to a particular theory, mutation, or isotype swapping, of the entire hinge region or CH2 region, or certain portions of a hinge region or CH2 region in an IgG1 results in the modified IgG1 having enhanced or altered properties relative to the IgG1 with a wild-type IgG1 constant region. For example, IgG1 can have residues 111-223 replaced with residues 111-220 of IgG4. Other non-limiting examples include IgG1 having at least 50%, at least 60%, at least 70%, at least 75%, at least 80%, at least 85%, at least 90%, at least 95%, or 100% residues 111-223 replaced with at least 50%, at least 60%, at least 70%, at least 75%, at least 80%, at least 85%, at least 90%, at least 95%, or 100% residues 111-220 of IgG4.

In some embodiments, a variant Fc molecule is a hybrid Fc molecule that comprises sequences from at least two IgG isotypes. For example, a variant Fc molecule may comprise the CH2 or CH3 region from one or more other isotypes. For example, a variant Fc can be an IgG1/IgG4 Fc molecule.

In some embodiments, a variant Fc comprises a CH2 region swapped from another IgG isotype. Examples of CH2 regions include, but are not limited to:

SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
A (IgG1 CH2, SEQ ID NO: 528);
or
SVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISK
A (IgG4 CH2, SEQ ID NO: 529).

Provided herein are variant Fc molecules comprising variant Fc domains. Exemplary variant Fc molecules comprising variant Fc domains include an IgG1 hinge, a CH1 domain, a CH2 domain and a CH3 domain, wherein at least one amino acid residue is mutated, wherein the mutation is a substitution, an insertion, or a deletion. In some embodiments, the insertion can be 1-5 residues. In some embodiments, a variant Fc molecule comprises an IgG1 hinge and IgG4 CH2 domain. In some embodiments, a variant Fc molecule comprises a mutated IgG1 hinge and IgG4 CH2 domain. A variant Fc molecule may have effector function similar to that of wild-type IgG, or may be engineered to have enhanced effector function relative to that of the wild-type IgG. In some embodiments, a variant Fc molecule may have FcγRIIβ binding affinity similar to that of wild-type IgG. In some embodiments, a variant Fc molecule may have FcγRIIβ binding affinity that is enhanced to that of wild-type IgG. A variant Fc molecule may comprise a wild-type CH1, hinge, CH2 and/or CH3 domain, or a variant thereof, e.g., a CH1, hinge, CH2 and/or CH3 domain having one or more amino acid substitutions, deletions or additions relative to the corresponding wild-type domain, and/or having an amino acid sequence that is at least 70%, at least 75&, at least 80%, at least 85%, at least 90%, at least 91%, at least 92%, at least 93%, at least 94%, at least 95%, at least 96%, at least 97%, at least 98%, or at least 99% identical, or more, to the corresponding wild-type sequence.

In some embodiments, a variant Fc molecule comprises a mutation that confers selective binding to FcγRIIβ over FcγRIIα. As used herein, in reference to FcγRIIβ, the term “selective binding” means that the Fc polypeptide binds preferentially to FcγRIIβ over FcγRIIα, that is with a higher affinity to FcγRIIβ over FcγRIIα. Examples of such mutations are provided for in, for example, U.S. Pat. Nos. 7,662,926, 7,655,229, US 2009/0087428, U.S. Pat. No. 10,919,952, US 2007/0253948, and US 2006/0073142, each of which is hereby incorporated by reference in its entirety, including the specific mutations that are descried that affect FcγRIIβ binding. In some embodiments, the mutation is as described in Shields et al., J. Biol. Chem. 2001, 276:6591-6604, which is hereby incorporated by reference in its entirety.

In some embodiments, the polypeptide comprises an Fc domain as an effector domain to modulate the subject's response to the polypeptides, which can comprise a bifunctional antibody (two antigen binding domains that bind to the same or different targets as provided for herein). In some embodiments, the Fc polypeptide comprises a mutation that selectively binds to FcγRIIb.

In some embodiments, the Fc polypeptide comprises a mutation that selectively binds to FcγRIIb over FcγRIIα. As used herein, in reference to FcγRIIb, the term “selectively binds to” means that the Fc polypeptide binds preferentially to FcγRIIb, that is with a higher affinity to FcγRIIb as compared to other Fcγ receptors, such as FcγRIIα. Examples of such mutations are provided for in, for example, U.S. Pat. Nos. 7,662,926, 7,655,229, US 2009/0087428, US 2007/0253948, and US 2006/0073142, each of which is hereby incorporated by reference in its entirety, including the specific mutations that are descried that affect the FcγRIIb or FcγRIIα binding. In some embodiments, the mutation is as described in Shields et al., J. Biol. Chem. 2001, 276:6591-6604, which is hereby incorporated by reference in its entirety, including the specific mutations that are described and that affect the FcγRIIb or FcγRIIa binding. In some embodiments, the mutations in the Fc polypeptide are at positions S298, E333, or K334, or any combination thereof (numbering according to EU numbering). In some embodiments, the Fc polypeptide comprises a mutation that corresponds to S298A, E333A, or K334A, or any combination thereof. In some embodiments, the Fc polypeptide comprises the mutations of S298A, E333A, and K334A. In some embodiments, the mutations correspond to G236A, 1332E, G236A, S239D, or 1332E, or any combination thereof. In some embodiments, the Fc polypeptide comprises the mutations of G236A, 1332E, G236A, S239D, and 1332E. The mutations can also be as provided for in, Richards et al., Mol Cancer Ther 2008; 7 (8). August 2008, which is hereby incorporated by reference in its entirety, including the specific mutations that are descried that affect the FcγRIIb or FcγRIIα binding. In some embodiments, the Fc polypeptide comprises a N235S or L328F mutation. In some embodiments, the Fc polypeptide comprises a N235S and L328F mutation. The mutations can also be as provided for in Shang et al., The Journal of Biological Chemistry VOL. 289, NO. 22, pp. 15309-15318, May 30, 2014, which is hereby incorporated by reference in its entirety, including the specific mutations that are descried that affect the FcγRIIb or FcγRIIα binding.

In some embodiments, the Fc mutation is as described in U.S. Pat. No. 10,618,965; EP Serial No. 2679681; EP Serial No. 3604330, US 2014/0093496, US 2015/0203577, U.S. Pat. No. 9,540,451, EP Serial No. 2331578; EP Serial No. 3190128; U.S. Pat. No. 9,902,773; EP. Serial No. 3342782, U.S. Publication No. 2020/0332024; EP Serial No. 2796469; EP Serial No. 2331578; EP Serial No. 3190128; U.S. Pat. No. 9,902,773, EP Serial No. 2331578; EP Serial No. 3190128, U.S. Pat. Nos. 9,493,578, 9,394,366, 9,914,778, EP Serial No. 2940043, U.S. Pat. No. 9,890,218, EP Serial No. 2940135; U.S. Pat. Nos. 10,766,960, 10,919,953, EP 3721900, EP2889377, US 2016/0039912, EP 2982689, or EP 3783017, each of which is hereby incorporated by reference in its entirety, including the specific mutations that are descried that affect the FcγRIIb or FcγRIIα binding.

In some embodiments, the Fc polypeptide comprises a mutation, mutations, or a mutation set that increases selectivity for FcγRIIb. In some embodiments, the Fc polypeptide comprises a mutation, mutations, or a mutation set that increases affinity for FcγRIIb. In some embodiments, the Fc polypeptide comprises a mutation, mutations, or a mutation set that increases selectivity and affinity for FcγRIIb. In some embodiments, the Fc polypeptide comprises a mutation, mutations, or a mutation set that increases selectivity for FcγRIIb over FcγRIIα. In some embodiments, the Fc polypeptide comprises a mutation, mutations, or a mutation set that increases affinity for FcγRIIb over FcγRIIα. In some embodiments, the Fc polypeptide comprises a mutation, mutations, or a mutation set that increases selectivity and affinity for FcγRIIb over FcγRIIα. In some embodiments, the mutation, mutations, or the mutation set is such as those described herein.

In some embodiments, the Fc polypeptide comprises a mutation, mutations, or a mutation set, of G237E and P238E; P238D; P238D and E233D; P238D and L234W; P238D and L234Y; P238D and G237W; P238D and G237F; P238D and G237A; P238D and G237D; P238D and G237E; P238D and G237L; P238D and G237M; P238D and G237Y; P238D and S239D; P238D and S267V; P238D and S267Q; P238D and S267A; P238D and H268N; P238D and H268D; P238D and H268E; P238D and P271G; P238D and Y296D; P238D and V323I; P238D and V323L; P238D and V323M; P238D and K326L; P238D and K326Q; P238D and K326E; P238D and K326M; P238D and K326D; P238D and K326S; P238D and K326T; P238D and K326A; P238D and K326N; P238D and L328E; P238D and A330K; P238D and A330R; P238D and A330M; S239P; S239P and P230E; S239P and A231D; S239P and P232E; S239P and P238E; S239P, P230E and A231D; S239P, P230E and P232E; S239P, P230E and P238E; S239P, P230E, A231D and P232E; S239P, P230E, A231D and P238E; S239P, P230E, A231D, P232E and P238E; S239P, A231D and P232E; S239P, A231D and P238E; S239P, A231D, P232E and P238E; S239P, P232E and P238E; S267E; S267D; S267E and L328F; G236D and S267E; S239D and S267E; S239D and 1332E; K409E; L368K; S364D and K370G; S364Y and K370R; S364D; Y349K; K409D; K392E; D399K; S364E; L368E and K409E; S364E and F405A; Y349K and T394F; S364H and Y349K; P395T, V397S and F405A; T394F; T394S, P395V, P396T, V397E and F405S; V397S and F405A; S364H, D401K and F405A; Y349T, T394F and T411E; L351K, S364H and D401K; Y349T, L351E and T411E; S364H; Y349T; S364H and D401K; Y349T and T411E; S364H and T394F; Y349T and F405A; S364H and F405A; Y349T and T394F; F405A; S364E and T394F; Y349K and F405A; V397T and F405S; S364E and F405S; Y349K and T394Y; S364E, T411E and F405A; Y349K, T394F and D401K; S364E and T411E; Y349K and D401K; L351E and S364D; Y349K and L351K; L351E and S364E; Y349C and S364E; Y349K and S354C; S364H, F405A and T411E; Y349T, T394F and D401K; S364D and T394F; L235Y; L235R; G236D; L328F; L235Y, G236D, S267D and L328F; L235Y, G236D and S267D; L235Y, G236D and S267E; L235Y and G236D; L235Y, S267D and L328F; L235Y, S267E and L328F; L235Y and L328F; L235R, G236D, S267D and L328F; L235R, G236D and S267D; L235R, G236D and S267E; L235R and G236D; L235R, S267D and L328F; L235R, S267E and L328F; L235R and L328F; G236D, S267E and L328F; G236D, S267D and L328F; G236D and L328F; S267D and L328F; G236N and S267E; G236N; L234Y, L235Y, G236W, H268D and S298A; L234Y, L235Y, G236W, H268D, D270E and S298A; L234Y, L235Q, G236W, S239M, H268D, D270E and S298A; L234Y, L235Y, G236W, H268D, S298A and A327D; L234Y, L235Y, G236W, S239M, H268D, S298A and A327D; L234Y, L235Y, G236W, S239M, H268D, S298A, A327D, L328W and K334L; second IgG1 CH2 Domain; K326D, A330M and K334E; D270E, K326D, A330M and K334E; D270E, K326D, A330K and K334E; L234E, L235Y, G236W, S239M, H268D, S298A and A327D; L234S, L235Y, G236W, S239M, H268D, S298A and A327D; L235Q, G236W, S239M, H268D, D270E and S298A; L235Y, G236W, S239M, H268D, S298A and A327D; L234S, L235Q, G236W, S239M, H268D, D270E and S298A; L234F, L235Q, G236W, S239M, H268D, D270E and S298A; L234E, L235Q, G236W, S239M, H268D, D270E and S298A; L234F, L235Y, G236W, S239M, H268D, S298A and A327D; L234V, L235Q, G236W, S239M, H268D, D270E and S298A; L234D, L235Q, G236W, S239M, H268D, D270E and S298A; L234Q, L235Q, G236W, S239M, H268D, D270E and S298A; L234I, L235Q, G236W, S239M, H268D, D270E and S298A; L234M, L235Q, G236W, S239M, H268D, D270E and S298A; L234T, L235Q, G236W, S239M, H268D, D270E and S298A; L234A, L235Q, G236W, S239M, H268D, D270E and S298A; L234G, L235Q, G236W, S239M, H268D, D270E and S298A; L234H, L235Q, G236W, S239M, H268D, D270E and S298A; L234V, L235Y, G236W, S239M, H268D, S298A and A327D; L234D, L235Y, G236W, S239M, H268D, S298A and A327D; L234Q, L235Y, G236W, S239M, H268D, S298A and A327D; L234I, L235Y, G236W, S239M, H268D, S298A and A327D; L234M, L235Y, G236W, S239M, H268D, S298A and A327D; L234T, L235Y, G236W, S239M, H268D, S298A and A327D; L234A, L235Y, G236W, S239M, H268D, S298A and A327D; L234G, L235Y, G236W, S239M, H268D, S298A and A327D; L234H, L235Y, G236W, S239M, H268D, S298A and A327D; L234F, L235Q, G236W, S239I, H268D, D270E and S298A; L234E, L235Q, G236W, S239I, H268D, D270E and S298A; L234D, L235Q, G236W, S239I, H268D, D270E and S298A; L234V, L235Y, G236W, S239I, H268D, S298A and A327D; L234I and L235Y, G236W, S239I, H268D, S298A, A327D; L235Y, G236W, S239I, H268D, S298A, A327D; L234E, L235Y, G236W, S239I, H268D, S298A and A327D; L234D, L235Y, G236W, S239I, H268D, S298A and A327D; L234F, L235Y, G236W, S239I, H268D, S298A and A327D; L234T, L235Y, G236W, S239I, H268D, S298A and A327D; second polypeptide; D270E, K326D and K334E; D270E, K326D, A330F and K334E; D270E, K326D, A3301 and K334E; D270E, K326D, A330Y and K334E; D270E, K326D, A330H and K334E; P238D, E233D, G237D, H268D, P271G, Y296D and A330R; P238D, G237D, H268D, P271G, Y296D and A330R; P238D, G237D, H268E, P271G, Y296D and A330R; P238D, E233D, G237D, H268D, P271G, Y296D, A330R and 1332T; P238D, E233D, G237D, V264I, S267G, H268E, P271G and A330R; P238D, E233D, G237D, V264I, S267A, H268E, P271G and A330R; P238D, E233D, G237D, S267A, H268E, P271G, Y296D, A330R and 1332T; P238D, G237D, S267A, H268E, P271G, Y296D, A330R and 1332T; P238D, E233D, G237D, V264I, S267A, H268E and P271G; P238D, E233D, G237D, V264I, S267A, H268E, P271G, Y296D and A330R; P238D, E233D, G237D, V264I, S267A, H268E, P271G, Y296D, A330R and P396M; P238D, E233D, G237D, V264I, S267A, H268E, P271G, Y296D, A330R and P396L; P238D, G237D, V264I, S267A, H268E, P271G and A330R; P238D, G237D, V264I, S267A, H268E, P271G, Y296D and A330R; P238D, V264I, S267A, H268E and P271G; P238D, V264I, S267A, H268E, P271G and Y296D; P238D, G237D, S267A, H268E, P271G, Y296D and A330R; P238D, G237D, S267G, H268E, P271G, Y296D and A330R; P238D, E233D, G237D, V264I, S267A, H268E, P271G, A330R and P396M; P238D, E233D, G237D, V264I, S267A, H268E, P271G, A330R and P396L; P238D, E233D, G237D, V264I, S267A, H268E, P271G, Y296D, A327G, A330R and P396M; P238D, E233D, G237D, V264I, S267A, H268E, P271G, E272D and Y296D; P238D, G237D, V264I, S267A, H268E, P271G, E272P and A330R; P238D, G237D, V264I, S267A, H268E, P271G, E272P, Y296D and A330R; P238D, E233D, V264I, S267A, H268E and P271G; P238D, G237D, S267E, H268D, P271G, Y296D and A330R; P238D, V264I, S267A, H268E, P271G, E272D and Y296D; P238D, E233D, V264I, S267A, H268E, P271G and Y296D; P238D, E233D, L234Y, L235F, G237D, V264I, D265E, V266F, S267A, H268D, E269D, P271G, E272D, K274Q, Y296D, K326A, A327G, A330K, P331S, 1332K, E333K, K334R, R355A, D356E, L358M, P396A, K409R and Q419E; G237Q, P238D, F241M, Y296E, A330H and S324H; G237Q, P238D, F241M, H268P, Y296E and A330H; G237Q, P238D, L235F, F241M, Y296E and S324H; G237Q, P238D, L235F, F241M, H268P and Y296E; G237Q, P238D, F241M, H268P, Y296E and S324H; G237Q, P238D, L235F, F241M, H268P, Y296E and S324H; G237Q, P238D, L235F, F241M, Y296E, S324H and A330H; G237Q, P238D, L235F, F241M, H268P, Y296E and A330H; G237Q, P238D, F241M, H268P, Y296E, S324H and A330H; G237Q, P238D, E233D, V264I, S267R, H268P, P271G and Y296E; G237Q, P238D, F241M and Y296E; G237Q, P238D, F241M, Y296E and A330H; G237Q, P238D, L235F, F241M and Y296E; G237Q, P238D, L235F, F241M, Y296E and A330H; G237Q and P238D; P238D and F241M; P238D and F241L; P238D and H268P; P238D and Q295V; P238D and Y296E; P238D and Y296H; P238D and S298M; P238D and S324N; P238D and S324H; P238D and A330H; P238D and A330Y; P238D and F241M, H268P, Y296E and S324H; G237Q, P238D, F241M, Y296E and A330H; L235F, G237Q, P238D, F241M and Y296E; P238D, P271G and E233D; P238D, P271G and L234R; P238D, P271G and G237D; P238D, P271G and G237K; P238D, P271G and V264I; P238D, P271G and S267A; P238D, P271G and H268E; P238D, P271G and H268P; P238D, P271G and Y296D; P238D, P271G and Y296E; P238D, P271G, E233D, L234K, V264I, S267A and H268E; P238D, P271G, E233D, L234R, V264I, S267A and H268E; P238D, P271G, E233D, G237K, V264I, S267A and H268E; P238D, P271G, E233D, V264I, D265N, S267A and H268E; P238D, P271G, E233D, V264I, S267R and H268E; P238D, P271G, E233D, G237D, V264I, S267Y, H268E, Y296D, A330R and P396M; P238D, P271G, E233D, G237D, V264I, S267A, H268E, Y296D/Y296A, A330R and P396M; P238D, P271G, E233D, V264I, S267R, H268E and Y296E; P238D, P271G, E233D, V264I, S267R and H268P; P238D, P271G, E233D, F241M, V264I, S267R and H268E; P238D, P271G, E233D, V264I, S267R, H268P and Y296E; P238D, P271G, E233D, G237Q, V264I, S267R, H268P and Y296E; E233D, G237D, P238D, H268D, P271G, and A330R.

In some embodiments, the Fc polypeptide comprises a mutation or mutations of G237E and P238E. In some embodiments, the Fc polypeptide comprises a mutation of P238D. In some embodiments, the Fc polypeptide comprises a mutation or mutations of P238D and E233D. In some embodiments, the Fc polypeptide comprises a mutation or mutations of P238D and L234W. In some embodiments, the Fc polypeptide comprises a mutation or mutations of P238D and L234Y. In some embodiments, the Fc polypeptide comprises a mutation or mutations of P238D and G237W. In some embodiments, the Fc polypeptide comprises a mutation or mutations of P238D and G237F. In some embodiments, the Fc polypeptide comprises a mutation or mutations of P238D and G237A. In some embodiments, the Fc polypeptide comprises a mutation or mutations of P238D and G237D. In some embodiments, the Fc polypeptide comprises a mutation or mutations of P238D and G237E. In some embodiments, the Fc polypeptide comprises a mutation or mutations of P238D and G237L. In some embodiments, the Fc polypeptide comprises a mutation or mutations of P238D and G237M. In some embodiments, the Fc polypeptide comprises a mutation or mutations of P238D and G237Y. In some embodiments, the Fc polypeptide comprises a mutation or mutations of P238D and S239D. In some embodiments, the Fc polypeptide comprises a mutation or mutations of P238D and S267V. In some embodiments, the Fc polypeptide comprises a mutation or mutations of P238D and S267Q. In some embodiments, the Fc polypeptide comprises a mutation or mutations of P238D and S267A. In some embodiments, the Fc polypeptide comprises a mutation or mutations of P238D and H268N. In some embodiments, the Fc polypeptide comprises a mutation or mutations of P238D and H268D. In some embodiments, the Fc polypeptide comprises a mutation or mutations of P238D and H268E. In some embodiments, the Fc polypeptide comprises a mutation or mutations of P238D and P271G. In some embodiments, the Fc polypeptide comprises a mutation or mutations of P238D and Y296D. In some embodiments, the Fc polypeptide comprises a mutation or mutations of P238D and V323I. In some embodiments, the Fc polypeptide comprises a mutation or mutations of P238D and V323L. In some embodiments, the Fc polypeptide comprises a mutation or mutations of P238D and V323M. In some embodiments, the Fc polypeptide comprises a mutation or mutations of P238D and K326L. In some embodiments, the Fc polypeptide comprises a mutation or mutations of P238D and K326Q. In some embodiments, the Fc polypeptide comprises a mutation or mutations of P238D and K326E. In some embodiments, the Fc polypeptide comprises a mutation or mutations of P238D and K326M. In some embodiments, the Fc polypeptide comprises a mutation or mutations of P238D and K326D. In some embodiments, the Fc polypeptide comprises a mutation or mutations of P238D and K326S. In some embodiments, the Fc polypeptide comprises a mutation or mutations of P238D and K326T. In some embodiments, the Fc polypeptide comprises a mutation or mutations of P238D and K326A. In some embodiments, the Fc polypeptide comprises a mutation or mutations of P238D and K326N. In some embodiments, the Fc polypeptide comprises a mutation or mutations of P238D and L328E. In some embodiments, the Fc polypeptide comprises a mutation or mutations of P238D and A330K. In some embodiments, the Fc polypeptide comprises a mutation or mutations of P238D and A330R. In some embodiments, the Fc polypeptide comprises a mutation or mutations of P238D and A330M. In some embodiments, the Fc polypeptide comprises a mutation of S239P. In some embodiments, the Fc polypeptide comprises a mutation or mutations of S239P and P230E. In some embodiments, the Fc polypeptide comprises a mutation or mutations of S239P and A231D. In some embodiments, the Fc polypeptide comprises a mutation or mutations of S239P and P232E. In some embodiments, the Fc polypeptide comprises a mutation or mutations of S239P and P238E. In some embodiments, the Fc polypeptide comprises a mutation or mutations of S239P, P230E and A231D. In some embodiments, the Fc polypeptide comprises a mutation or mutations of S239P, P230E and P232E. In some embodiments, the Fc polypeptide comprises a mutation or mutations of S239P, P230E and P238E. In some embodiments, the Fc polypeptide comprises a mutation or mutations of S239P, P230E, A231D and P232E. In some embodiments, the Fc polypeptide comprises a mutation or mutations of S239P, P230E, A231D and P238E. In some embodiments, the Fc polypeptide comprises a mutation or mutations of S239P, P230E, A231D, P232E and P238E. In some embodiments, the Fc polypeptide comprises a mutation or mutations of S239P, A231D and P232E. In some embodiments, the Fc polypeptide comprises a mutation or mutations of S239P, A231D and P238E. In some embodiments, the Fc polypeptide comprises a mutation or mutations of S239P, A231D, P232E and P238E. In some embodiments, the Fc polypeptide comprises a mutation or mutations of S239P, P232E and P238E. In some embodiments, the Fc polypeptide comprises a mutation of S267E. In some embodiments, the Fc polypeptide comprises a mutation of S267D. In some embodiments, the Fc polypeptide comprises a mutation or mutations of S267E and L328F. In some embodiments, the Fc polypeptide comprises a mutation or mutations of G236D and S267E. In some embodiments, the Fc polypeptide comprises a mutation or mutations of S239D and S267E. In some embodiments, the Fc polypeptide comprises a mutation or mutations of S239D and 1332E. In some embodiments, the Fc polypeptide comprises a mutation of K409E. In some embodiments, the Fc polypeptide comprises a mutation of L368K. In some embodiments, the Fc polypeptide comprises a mutation or mutations of S364D and K370G. In some embodiments, the Fc polypeptide comprises a mutation or mutations of S364Y and K370R. In some embodiments, the Fc polypeptide comprises a mutation of S364D. In some embodiments, the Fc polypeptide comprises a mutation of Y349K. In some embodiments, the Fc polypeptide comprises a mutation of K409D. In some embodiments, the Fc polypeptide comprises a mutation of K392E. In some embodiments, the Fc polypeptide comprises a mutation of D399K. In some embodiments, the Fc polypeptide comprises a mutation of S364E. In some embodiments, the Fc polypeptide comprises a mutation or mutations of L368E and K409E. In some embodiments, the Fc polypeptide comprises a mutation or mutations of S364E and F405A. In some embodiments, the Fc polypeptide comprises a mutation or mutations of Y349K and T394F. In some embodiments, the Fc polypeptide comprises a mutation or mutations of S364H and Y349K. In some embodiments, the Fc polypeptide comprises a mutation or mutations of P395T, V397S and F405A. In some embodiments, the Fc polypeptide comprises a mutation of T394F. In some embodiments, the Fc polypeptide comprises a mutation or mutations of T394S, P395V, P396T, V397E and F405S. In some embodiments, the Fc polypeptide comprises a mutation or mutations of V397S and F405A. In some embodiments, the Fc polypeptide comprises a mutation or mutations of S364H, D401K and F405A. In some embodiments, the Fc polypeptide comprises a mutation or mutations of Y349T, T394F and T411E. In some embodiments, the Fc polypeptide comprises a mutation or mutations of L351K, S364H and D401K. In some embodiments, the Fc polypeptide comprises a mutation or mutations of Y349T, L351E and T411E. In some embodiments, the Fc polypeptide comprises a mutation of S364H. In some embodiments, the Fc polypeptide comprises a mutation of Y349T. In some embodiments, the Fc polypeptide comprises a mutation or mutations of S364H and D401K. In some embodiments, the Fc polypeptide comprises a mutation or mutations of Y349T and T411E. In some embodiments, the Fc polypeptide comprises a mutation or mutations of S364H and T394F. In some embodiments, the Fc polypeptide comprises a mutation or mutations of Y349T and F405A. In some embodiments, the Fc polypeptide comprises a mutation or mutations of S364H and F405A. In some embodiments, the Fc polypeptide comprises a mutation or mutations of Y349T and T394F. In some embodiments, the Fc polypeptide comprises a mutation of F405A. In some embodiments, the Fc polypeptide comprises a mutation or mutations of S364E and T394F. In some embodiments, the Fc polypeptide comprises a mutation or mutations of Y349K and F405A. In some embodiments, the Fc polypeptide comprises a mutation or mutations of V397T and F405S. In some embodiments, the Fc polypeptide comprises a mutation or mutations of S364E and F405S. In some embodiments, the Fc polypeptide comprises a mutation or mutations of Y349K and T394Y. In some embodiments, the Fc polypeptide comprises a mutation or mutations of S364E, T411E and F405A. In some embodiments, the Fc polypeptide comprises a mutation or mutations of Y349K, T394F and D401K. In some embodiments, the Fc polypeptide comprises a mutation or mutations of S364E and T411E. In some embodiments, the Fc polypeptide comprises a mutation or mutations of Y349K and D401K. In some embodiments, the Fc polypeptide comprises a mutation or mutations of L351E and S364D. In some embodiments, the Fc polypeptide comprises a mutation or mutations of Y349K and L351K. In some embodiments, the Fc polypeptide comprises a mutation or mutations of L351E and S364E. In some embodiments, the Fc polypeptide comprises a mutation or mutations of Y349C and S364E. In some embodiments, the Fc polypeptide comprises a mutation or mutations of Y349K and S354C. In some embodiments, the Fc polypeptide comprises a mutation or mutations of S364H, F405A and T411E. In some embodiments, the Fc polypeptide comprises a mutation or mutations of Y349T, T394F and D401K. In some embodiments, the Fc polypeptide comprises a mutation or mutations of S364D and T394F. In some embodiments, the Fc polypeptide comprises a mutation of L235Y. In some embodiments, the Fc polypeptide comprises a mutation of L235R. In some embodiments, the Fc polypeptide comprises a mutation of G236D. In some embodiments, the Fc polypeptide comprises a mutation of L328F. In some embodiments, the Fc polypeptide comprises a mutation or mutations of L235Y, G236D, S267D and L328F. In some embodiments, the Fc polypeptide comprises a mutation or mutations of L235Y, G236D and S267D. In some embodiments, the Fc polypeptide comprises a mutation or mutations of L235Y, G236D and S267E. In some embodiments, the Fc polypeptide comprises a mutation or mutations of L235Y and G236D. In some embodiments, the Fc polypeptide comprises a mutation or mutations of L235Y, S267D and L328F. In some embodiments, the Fc polypeptide comprises a mutation or mutations of L235Y, S267E and L328F. In some embodiments, the Fc polypeptide comprises a mutation or mutations of L235Y and L328F. In some embodiments, the Fc polypeptide comprises a mutation or mutations of L235R, G236D, S267D and L328F. In some embodiments, the Fc polypeptide comprises a mutation or mutations of L235R, G236D and S267D. In some embodiments, the Fc polypeptide comprises a mutation or mutations of L235R, G236D and S267E. In some embodiments, the Fc polypeptide comprises a mutation or mutations of L235R and G236D. In some embodiments, the Fc polypeptide comprises a mutation or mutations of L235R, S267D and L328F. In some embodiments, the Fc polypeptide comprises a mutation or mutations of L235R, S267E and L328F. In some embodiments, the Fc polypeptide comprises a mutation or mutations of L235R and L328F. In some embodiments, the Fc polypeptide comprises a mutation or mutations of G236D, S267E and L328F. In some embodiments, the Fc polypeptide comprises a mutation or mutations of G236D, S267D and L328F. In some embodiments, the Fc polypeptide comprises a mutation or mutations of G236D and L328F. In some embodiments, the Fc polypeptide comprises a mutation or mutations of S267D and L328F. In some embodiments, the Fc polypeptide comprises a mutation or mutations of G236N and S267E. In some embodiments, the Fc polypeptide comprises a mutation of G236N.

In some embodiments, the Fc polypeptide comprises a mutation or mutations of L234Y, L235Y, G236W, H268D and S298A. In some embodiments, the Fc polypeptide comprises a mutation or mutations of L234Y, L235Y, G236W, H268D, D270E and S298A. In some embodiments, the Fc polypeptide comprises a mutation or mutations of L234Y, L235Q, G236W, S239M, H268D, D270E and S298A. In some embodiments, the Fc polypeptide comprises a mutation or mutations of L234Y, L235Y, G236W, H268D, S298A and A327D. In some embodiments, the Fc polypeptide comprises a mutation or mutations of L234Y, L235Y, G236W, S239M, H268D, S298A and A327D. In some embodiments, the Fc polypeptide comprises a mutation or mutations of L234Y, L235Y, G236W, S239M, H268D, S298A, A327D, L328W and K334L. In some embodiments, the Fc polypeptide comprises a mutation or mutations of second IgG1 CH2 Domain. In some embodiments, the Fc polypeptide comprises a mutation or mutations of K326D, A330M and K334E. In some embodiments, the Fc polypeptide comprises a mutation or mutations of D270E, K326D, A330M and K334E. In some embodiments, the Fc polypeptide comprises a mutation or mutations of D270E, K326D, A330K and K334E. In some embodiments, the Fc polypeptide comprises a mutation or mutations of L234E, L235Y, G236W, S239M, H268D, S298A and A327D. In some embodiments, the Fc polypeptide comprises a mutation or mutations of L234S, L235Y, G236W, S239M, H268D, S298A and A327D. In some embodiments, the Fc polypeptide comprises a mutation or mutations of L235Q, G236W, S239M, H268D, D270E and S298A. In some embodiments, the Fc polypeptide comprises a mutation or mutations of L235Y, G236W, S239M, H268D, S298A and A327D. In some embodiments, the Fc polypeptide comprises a mutation or mutations of L234S, L235Q, G236W, S239M, H268D, D270E and S298A. In some embodiments, the Fc polypeptide comprises a mutation or mutations of L234F, L235Q, G236W, S239M, H268D, D270E and S298A. In some embodiments, the Fc polypeptide comprises a mutation or mutations of L234E, L235Q, G236W, S239M, H268D, D270E and S298A. In some embodiments, the Fc polypeptide comprises a mutation or mutations of L234F, L235Y, G236W, S239M, H268D, S298A and A327D. In some embodiments, the Fc polypeptide comprises a mutation or mutations of L234V, L235Q, G236W, S239M, H268D, D270E and S298A. In some embodiments, the Fc polypeptide comprises a mutation or mutations of L234D, L235Q, G236W, S239M, H268D, D270E and S298A. In some embodiments, the Fc polypeptide comprises a mutation or mutations of L234Q, L235Q, G236W, S239M, H268D, D270E and S298A. In some embodiments, the Fc polypeptide comprises a mutation or mutations of L234I, L235Q, G236W, S239M, H268D, D270E and S298A. In some embodiments, the Fc polypeptide comprises a mutation or mutations of L234M, L235Q, G236W, S239M, H268D, D270E and S298A. In some embodiments, the Fc polypeptide comprises a mutation or mutations of L234T, L235Q, G236W, S239M, H268D, D270E and S298A. In some embodiments, the Fc polypeptide comprises a mutation or mutations of L234A, L235Q, G236W, S239M, H268D, D270E and S298A. In some embodiments, the Fc polypeptide comprises a mutation or mutations of L234G, L235Q, G236W, S239M, H268D, D270E and S298A. In some embodiments, the Fc polypeptide comprises a mutation or mutations of L234H, L235Q, G236W, S239M, H268D, D270E and S298A. In some embodiments, the Fc polypeptide comprises a mutation or mutations of L234V, L235Y, G236W, S239M, H268D, S298A and A327D. In some embodiments, the Fc polypeptide comprises a mutation or mutations of L234D, L235Y, G236W, S239M, H268D, S298A and A327D. In some embodiments, the Fc polypeptide comprises a mutation or mutations of L234Q, L235Y, G236W, S239M, H268D, S298A and A327D. In some embodiments, the Fc polypeptide comprises a mutation or mutations of L234I, L235Y, G236W, S239M, H268D, S298A and A327D. In some embodiments, the Fc polypeptide comprises a mutation or mutations of L234M, L235Y, G236W, S239M, H268D, S298A and A327D. In some embodiments, the Fc polypeptide comprises a mutation or mutations of L234T, L235Y, G236W, S239M, H268D, S298A and A327D. In some embodiments, the Fc polypeptide comprises a mutation or mutations of L234A, L235Y, G236W, S239M, H268D, S298A and A327D. In some embodiments, the Fc polypeptide comprises a mutation or mutations of L234G, L235Y, G236W, S239M, H268D, S298A and A327D. In some embodiments, the Fc polypeptide comprises a mutation or mutations of L234H, L235Y, G236W, S239M, H268D, S298A and A327D. In some embodiments, the Fc polypeptide comprises a mutation or mutations of L234F, L235Q, G236W, S239I, H268D, D270E and S298A. In some embodiments, the Fc polypeptide comprises a mutation or mutations of L234E, L235Q, G236W, S239I, H268D, D270E and S298A. In some embodiments, the Fc polypeptide comprises a mutation or mutations of L234D, L235Q, G236W, S239I, H268D, D270E and S298A. In some embodiments, the Fc polypeptide comprises a mutation or mutations of L234V, L235Y, G236W, S239I, H268D, S298A and A327D. In some embodiments, the Fc polypeptide comprises a mutation or mutations of L234I and L235Y, G236W, S239I, H268D, S298A, A327D. In some embodiments, the Fc polypeptide comprises a mutation or mutations of L235Y, G236W, S239I, H268D, S298A, A327D. In some embodiments, the Fc polypeptide comprises a mutation or mutations of L234E, L235Y, G236W, S239I, H268D, S298A and A327D. In some embodiments, the Fc polypeptide comprises a mutation or mutations of L234D, L235Y, G236W, S239I, H268D, S298A and A327D. In some embodiments, the Fc polypeptide comprises a mutation or mutations of L234F, L235Y, G236W, S239I, H268D, S298A and A327D. In some embodiments, the Fc polypeptide comprises a mutation or mutations of L234T, L235Y, G236W, S239I, H268D, S298A and A327D. In some embodiments, the Fc polypeptide comprises a mutation or mutations of second polypeptide. In some embodiments, the Fc polypeptide comprises a mutation or mutations of D270E, K326D and K334E. In some embodiments, the Fc polypeptide comprises a mutation or mutations of D270E, K326D, A330F and K334E. In some embodiments, the Fc polypeptide comprises a mutation or mutations of D270E, K326D, A3301 and K334E. In some embodiments, the Fc polypeptide comprises a mutation or mutations of D270E, K326D, A330Y and K334E. In some embodiments, the Fc polypeptide comprises a mutation or mutations of D270E, K326D, A330H and K334E.

In some embodiments, the Fc polypeptide comprises a mutation or mutations of P238D, E233D, G237D, H268D, P271G, Y296D and A330R. In some embodiments, the Fc polypeptide comprises a mutation or mutations of P238D, G237D, H268D, P271G, Y296D and A330R. In some embodiments, the Fc polypeptide comprises a mutation or mutations of P238D, G237D, H268E, P271G, Y296D and A330R. In some embodiments, the Fc polypeptide comprises a mutation or mutations of P238D, E233D, G237D, H268D, P271G, Y296D, A330R and 1332T. In some embodiments, the Fc polypeptide comprises a mutation or mutations of P238D, E233D, G237D, V264I, S267G, H268E, P271G and A330R. In some embodiments, the Fc polypeptide comprises a mutation or mutations of P238D, E233D, G237D, V264I, S267A, H268E, P271G and A330R. In some embodiments, the Fc polypeptide comprises a mutation or mutations of P238D, E233D, G237D, S267A, H268E, P271G, Y296D, A330R and 1332T. In some embodiments, the Fc polypeptide comprises a mutation or mutations of P238D, G237D, S267A, H268E, P271G, Y296D, A330R and 1332T. In some embodiments, the Fc polypeptide comprises a mutation or mutations of P238D, E233D, G237D, V264I, S267A, H268E and P271G. In some embodiments, the Fc polypeptide comprises a mutation or mutations of P238D, E233D, G237D, V264I, S267A, H268E, P271G, Y296D and A330R. In some embodiments, the Fc polypeptide comprises a mutation or mutations of P238D, E233D, G237D, V264I, S267A, H268E, P271G, Y296D, A330R and P396M. In some embodiments, the Fc polypeptide comprises a mutation or mutations of P238D, E233D, G237D, V264I, S267A, H268E, P271G, Y296D, A330R and P396L. In some embodiments, the Fc polypeptide comprises a mutation or mutations of P238D, G237D, V264I, S267A, H268E, P271G and A330R. In some embodiments, the Fc polypeptide comprises a mutation or mutations of P238D, G237D, V264I, S267A, H268E, P271G, Y296D and A330R. In some embodiments, the Fc polypeptide comprises a mutation or mutations of P238D, V264I, S267A, H268E and P271G. In some embodiments, the Fc polypeptide comprises a mutation or mutations of P238D, V264I, S267A, H268E, P271G and Y296D. In some embodiments, the Fc polypeptide comprises a mutation or mutations of P238D, G237D, S267A, H268E, P271G, Y296D and A330R. In some embodiments, the Fc polypeptide comprises a mutation or mutations of P238D, G237D, S267G, H268E, P271G, Y296D and A330R. In some embodiments, the Fc polypeptide comprises a mutation or mutations of P238D, E233D, G237D, V264I, S267A, H268E, P271G, A330R and P396M. In some embodiments, the Fc polypeptide comprises a mutation or mutations of P238D, E233D, G237D, V264I, S267A, H268E, P271G, A330R and P396L. In some embodiments, the Fc polypeptide comprises a mutation or mutations of P238D, E233D, G237D, V264I, S267A, H268E, P271G, Y296D, A327G, A330R and P396M. In some embodiments, the Fc polypeptide comprises a mutation or mutations of P238D, E233D, G237D, V264I, S267A, H268E, P271G, E272D and Y296D. In some embodiments, the Fc polypeptide comprises a mutation or mutations of P238D, G237D, V264I, S267A, H268E, P271G, E272P and A330R. In some embodiments, the Fc polypeptide comprises a mutation or mutations of P238D, G237D, V264I, S267A, H268E, P271G, E272P, Y296D and A330R. In some embodiments, the Fc polypeptide comprises a mutation or mutations of P238D, E233D, V264I, S267A, H268E and P271G. In some embodiments, the Fc polypeptide comprises a mutation or mutations of P238D, G237D, S267E, H268D, P271G, Y296D and A330R. In some embodiments, the Fc polypeptide comprises a mutation or mutations of P238D, V264I, S267A, H268E, P271G, E272D and Y296D. In some embodiments, the Fc polypeptide comprises a mutation or mutations of P238D, E233D, V264I, S267A, H268E, P271G and Y296D. In some embodiments, the Fc polypeptide comprises a mutation or mutations of P238D, E233D, L234Y, L235F, G237D, V264I, D265E, V266F, S267A, H268D, E269D, P271G, E272D, K274Q, Y296D, K326A, A327G, A330K, P331S, 1332K, E333K, K334R, R355A, D356E, L358M, P396A, K409R and Q419E.

In some embodiments, the Fc polypeptide comprises a mutation or mutations of G237Q, P238D, F241M, Y296E, A330H and S324H. In some embodiments, the Fc polypeptide comprises a mutation or mutations of G237Q, P238D, F241M, H268P, Y296E and A330H. In some embodiments, the Fc polypeptide comprises a mutation or mutations of G237Q, P238D, L235F, F241M, Y296E and S324H. In some embodiments, the Fc polypeptide comprises a mutation or mutations of G237Q, P238D, L235F, F241M, H268P and Y296E. In some embodiments, the Fc polypeptide comprises a mutation or mutations of G237Q, P238D, F241M, H268P, Y296E and S324H. In some embodiments, the Fc polypeptide comprises a mutation or mutations of G237Q, P238D, L235F, F241M, H268P, Y296E and S324H. In some embodiments, the Fc polypeptide comprises a mutation or mutations of G237Q, P238D, L235F, F241M, Y296E, S324H and A330H. In some embodiments, the Fc polypeptide comprises a mutation or mutations of G237Q, P238D, L235F, F241M, H268P, Y296E and A330H. In some embodiments, the Fc polypeptide comprises a mutation or mutations of G237Q, P238D, F241M, H268P, Y296E, S324H and A330H. In some embodiments, the Fc polypeptide comprises a mutation or mutations of G237Q, P238D, E233D, V264I, S267R, H268P, P271G and Y296E. In some embodiments, the Fc polypeptide comprises a mutation or mutations of G237Q, P238D, F241M and Y296E. In some embodiments, the Fc polypeptide comprises a mutation or mutations of G237Q, P238D, F241M, Y296E and A330H. In some embodiments, the Fc polypeptide comprises a mutation or mutations of G237Q, P238D, L235F, F241M and Y296E. In some embodiments, the Fc polypeptide comprises a mutation or mutations of G237Q, P238D, L235F, F241M, Y296E and A330H.

In some embodiments, the Fc polypeptide comprises a mutation or mutations of G237Q and P238D. In some embodiments, the Fc polypeptide comprises a mutation or mutations of P238D and F241M. In some embodiments, the Fc polypeptide comprises a mutation or mutations of P238D and F241L. In some embodiments, the Fc polypeptide comprises a mutation or mutations of P238D and H268P. In some embodiments, the Fc polypeptide comprises a mutation or mutations of P238D and Q295V. In some embodiments, the Fc polypeptide comprises a mutation or mutations of P238D and Y296E. In some embodiments, the Fc polypeptide comprises a mutation or mutations of P238D and Y296H. In some embodiments, the Fc polypeptide comprises a mutation or mutations of P238D and S298M. In some embodiments, the Fc polypeptide comprises a mutation or mutations of P238D and S324N. In some embodiments, the Fc polypeptide comprises a mutation or mutations of P238D and S324H. In some embodiments, the Fc polypeptide comprises a mutation or mutations of P238D and A330H. In some embodiments, the Fc polypeptide comprises a mutation or mutations of P238D and A330Y. In some embodiments, the Fc polypeptide comprises a mutation or mutations of P238D and F241M, H268P, Y296E and S324H. In some embodiments, the Fc polypeptide comprises a mutation or mutations of G237Q, P238D, F241M, Y296E and A330H. In some embodiments, the Fc polypeptide comprises a mutation or mutations of L235F, G237Q, P238D, F241M and Y296E.

In some embodiments, the Fc polypeptide comprises a mutation or mutations of P238D, P271G and E233D. In some embodiments, the Fc polypeptide comprises a mutation or mutations of P238D, P271G and L234R. In some embodiments, the Fc polypeptide comprises a mutation or mutations of P238D, P271G and G237D. In some embodiments, the Fc polypeptide comprises a mutation or mutations of P238D, P271G and G237K. In some embodiments, the Fc polypeptide comprises a mutation or mutations of P238D, P271G and V264I. In some embodiments, the Fc polypeptide comprises a mutation or mutations of P238D, P271G and S267A. In some embodiments, the Fc polypeptide comprises a mutation or mutations of P238D, P271G and H268E. In some embodiments, the Fc polypeptide comprises a mutation or mutations of P238D, P271G and H268P. In some embodiments, the Fc polypeptide comprises a mutation or mutations of P238D, P271G and Y296D. In some embodiments, the Fc polypeptide comprises a mutation or mutations of P238D, P271G and Y296E. In some embodiments, the Fc polypeptide comprises a mutation or mutations of P238D, P271G, E233D, L234K, V264I, S267A and H268E. In some embodiments, the Fc polypeptide comprises a mutation or mutations of P238D, P271G, E233D, L234R, V264I, S267A and H268E. In some embodiments, the Fc polypeptide comprises a mutation or mutations of P238D, P271G, E233D, G237K, V264I, S267A and H268E. In some embodiments, the Fc polypeptide comprises a mutation or mutations of P238D, P271G, E233D, V264I, D265N, S267A and H268E. In some embodiments, the Fc polypeptide comprises a mutation or mutations of P238D, P271G, E233D, V264I, S267R and H268E. In some embodiments, the Fc polypeptide comprises a mutation or mutations of P238D, P271G, E233D, G237D, V264I, S267Y, H268E, Y296D, A330R and P396M. In some embodiments, the Fc polypeptide comprises a mutation or mutations of P238D, P271G, E233D, G237D, V264I, S267A, H268E, Y296D/Y296A, A330R and P396M. In some embodiments, the Fc polypeptide comprises a mutation or mutations of P238D, P271G, E233D, V264I, S267R, H268E and Y296E. In some embodiments, the Fc polypeptide comprises a mutation or mutations of P238D, P271G, E233D, V264I, S267R and H268P. In some embodiments, the Fc polypeptide comprises a mutation or mutations of P238D, P271G, E233D, F241M, V264I, S267R and H268E. In some embodiments, the Fc polypeptide comprises a mutation or mutations of P238D, P271G, E233D, V264I, S267R, H268P and Y296E. In some embodiments, the Fc polypeptide comprises a mutation or mutations of P238D, P271G, E233D, G237Q, V264I, S267R, H268P and Y296E. In some embodiments, the Fc polypeptide comprises a mutation or mutations of E233D, G237D, P238D, H268D, P271G, and A330R.

In some embodiments, an Fc polypeptide comprises a polypeptide comprising one or more mutations that confers selective binding to FcγRIIβ over FcγRIIα. In some embodiments, an Fc polypeptide comprises one or more mutations that enhance selective binding to FcγRIIβ over FcγRIIα. In some embodiments, an Fc polypeptide comprises one or more mutations selected from mutations associated with any one of VFC-1 through VFC-89, as provided in Table 4 below, which are either recited in table or would immediately become apparent if aligned to the amino acid sequence of SEQ ID NO: 516 by either BLASTP or Clustal Omega with default settings.

TABLE 4

| ID | Fc Sequence | Other Comments |
|---|---|---|
| VFC-1 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGFSVFLFPPKPKDTLM | |
| | ISRTPEVTCVVVDVKHEDPEVKFNWYVDGVEVHNAKTKPREEQYN | |
| | STYRVVSVLTVLHQDWLNGKEYKCKVSNKAMPEPIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 417) | |
| VFC-2 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGFSVFLFPPKPKDTLM | |
| | ISRTPEVTCVVVDVKHEDPEVKFNWYVDGVEVHNAKTKPREEQYN | |
| | STYRVVSVLTVLHQDWLNGKEYKCKVSNKDMPEPIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 418) | |
| VFC-3 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGHFSVFLFPPKPKDTLM | |
| | ISRTPEVTCVVVDVKHEDPEVKFNWYVDGVEVHNAKTKPREEQYN | |
| | STYRVVSVLTVLHQDWLNGKEYKCKVSNKDMPEPIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 419) | |
| VFC-4 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGMFSVFLFPPKPKDTLM | |
| | ISRTPEVTCVVVDVKHEDPEVKFNWYVDGVEVHNAKTKPREEQYN | |
| | STYRVVSVLTVLHQDWLNGKEYKCKVSNKDMPEPIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 420) | |
| VFC-5 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 421) | |
| VFC-6 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELGQGPSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALRAPIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 422) | |
| VFC-7 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPEPGQGPSVFLFPPKPKDTLM | |
| | ISRTPEVTCVVVDVSHEDPEVKENWYVDGVEVHNAKTKPREEQYN | |
| | STYRVVSVLTVLHQDWLNGKEYKCKVSNKALRAPIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 423) | |
| VFC-8 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELGQGPSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKAWRAPIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 424) | |
| VFC-9 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELGQRPSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKAWRAPIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 425) | |
| VFC-10 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELGQGPSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALDAPIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 426) | |
| VFC-11 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPLKLGAPSVFLFPPKPKDTLM | |
| | ISRTPEVTCVVVDVSPENPEVKFNWYVDGVEVHNAKTKPREEQYN | |
| | STYRVVSVLTVLHQDWLNGKEYKCKVSTSTIPGQIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 427) | |
| VFC-12 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPMGGGAPSVFLFPPKPKDTLM | |
| | ISRTPEVTCVVVDVSPEDPEVEFNWYVDGVEVHNAKTKPREEQYN | |
| | STYRVVSVLTVLHQDWLNGKEYKCKVSTPSQPADIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 428) | |
| VFC-13 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPITPGAPSVFLFPPKPKDTLM | |
| | ISRTPEVTCVVVDVSPEAPEVEFNWYVDGVEVHNAKTKPREEQYN | |
| | STYRVVSVLTVLHQDWLNGKEYKCKVSTAGLGSNIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 429) | |
| VFC-14 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPRTPALPSVFLFPPKPKDTLM | |
| | ISRTPEVTCVVVDVSPEDPEVEFNWYVDGVEVHNAKTKPREEQYN | |
| | STYRVVSVLTVLHQDWLNGKEYKCKVSNGEIREHIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 430) | |
| VFC-15 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGLPSVFLFPPKPKDTLM | |
| | ISRTPEVTCVVVDVSHEDPEVKENWYVDGVEVHNAKTKPREEQYN | |
| | QTYRVVSVLTVLHQDWLNGKEYKCKVSNKDLPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 431) | |
| VFC-16 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGLPSVFLFPPKPKDTLM | |
| | ISRTPEVTCVVVDVSHEDPEVKFNWYVDGVEVHNAKTKPREEQYN | |
| | QRYRVVSVLTVLHQDWLNGKEYKCKVSNKDLPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 432) | |
| VFC-17 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLPLPSVFLFPPKPKDTLM | |
| | ISRTPEVTCVVVDVSHEDPEVKENWYVDGVEVHNAKTKPREEQYN | |
| | QTYRVVSVLTVLHQDWLNGKEYKCKVSNKDLPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 434) | |
| VFC-18 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGLPSVFLFPPKPKDTLM | |
| | ISRTPEVTCVVVDVSHEDPEVKFNWYVDGVEVHNAKTKPREEQYN | |
| | QTYRVVSVLTVLHQDWLNGKEYKCKVSDKDLPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 435) | |
| VFC-19 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPEMLPLPSVFLFPPKPKDTLM | |
| | ISRTPEVTCVVVDVSHEDPEVKFNWYVDGVEVHNAKTKPREEQYN | |
| | QTYRVVSVLTVLHQDWLNGKEYKCKVSNKDLPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 436) | |
| VFC-20 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPEMLPLPSVFLFPPKPKDTLM | |
| | ISRTPEVTCVVVDVSHEDPEVKFNWYVDGVEVHNAKTKPREEQYN | |
| | QRYRVVSVLTVLHQDWLNGKEYKCKVSNKDLPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 437) | |
| VFC-21 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPEMLPLPSVFLFPPKPKDTLM | |
| | ISRTPEVTCVVVDVSHEDPEVKFNWYVDGVEVHNAKTKPREEQYN | |
| | QTYRVVSVLTVLHQDWLNGKEYKCKVSDKDLPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 438) | |
| VFC-22 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVWDVSHEDPEVKENWYVDGVEVHNAKTKPREEQYN | |
| | STYRVVSVLTVLHQDWLNGKEYKCKVSNKDLPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 439) | |
| VFC-23 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVWDVSHEDPEVKFNWYVDGVEVHNAKTKPREEQYN | |
| | STYRVVSVLTVLHQDWLNGKEYKCKVSDKDLPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 450) | |
| VFC-24 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVWDHSHEDPEVKENWYVDGVEVHNAKTKPREEQYN | |
| | STYRVVSVLTVLHQDWLNGKEYKCKVSDKDLPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 451) | |
| VFC-25 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSQYDPEVKFNWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 452) | |
| VFC-26 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDASQYDPEVKFNWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 453) | |
| VFC-27 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVVDVSQYDPEVKENWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 454) | |
| VFC-28 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSNYDPEVKFNWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 455) | |
| VFC-29 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVVDVSQYEPEVKFNWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 456) | |
| VFC-30 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVVDASQYEPEVKENWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 457) | |
| VFC-31 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVVDVSNYEPEVKFNWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 458) | |
| VFC-32 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVVFVSQYEPEVKENWYVDGVEVHNAKTKPREEQNN | |
| | STYRVVSVLTVLHQDWLNGKEYKCKVSNKALPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 459) | |
| VFC-33 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPMKEGAPSVFLFPPKPKDTLM | |
| | ISRTPEVTCVVVDVSPKSPEVEFNWYVDGVEVHNAKTKPREEQYN | |
| | STYRVVSVLTVLHQDWLNGKEYKCKVSTSALAAEIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 460) | |
| VFC-34 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPPGPGSPSVFLFPPKPKDTLM | |
| | ISRTPEVTCVVVDVSPADPEVHENWYVDGVEVHNAKTKPREEQYN | |
| | STYRVVSVLTVLHQDWLNGKEYKCKVSNNALIGQIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 461) | |
| VFC-35 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPVISGGPSVFLFPPKPKDTLM | |
| | ISRTPEVTCVVVDVSPENPEVKENWYVDGVEVHNAKTKPREEQYN | |
| | STYRVVSVLTVLHQDWLNGKEYKCKVSTKNHPQPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 462) | |
| VFC-36 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPKLLGAPSVFLFPPKPKDTLM | |
| | ISRTPEVTCVVVDVSPSDPEVHFNWYVDGVEVHNAKTKPREEQYN | |
| | STYRVVSVLTVLHQDWLNGKEYKCKVSNKNVNGVIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 463) | |
| VFC-37 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSHEDPEVKENWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 464) | |
| VFC-38 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVWDHSHEDPEVKENWYVDGVEVHNAKTKPREEQYN | |
| | STYRVVSVLTVLHQDWLNGKEYKCKVSNKDLPAPIEKTISKAKGQ | |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 465) | |
| VFC-39 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGESVFLFPPKPKDTLM | |
| | ISRTPEVTCVVVDVSHEDPEVKENWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 466) | |
| VFC-40 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGEESVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 467) | |
| VFC-41 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGDESVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 468) | |
| VFC-42 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGNSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 469) | |
| VFC-43 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGENSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 470) | |
| VFC-44 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGDNSVFLFPPKPKDTLM | |
| | ISRTPEVTCVVVDVSHEDPEVKENWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 471) | |
| VFC-45 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGNSVFLFPPKPKDTLM | |
| | ISRTPEVTCVVVDVKHEDPEVKFNWYVDGVEVHNAKTKPREEQYN | |
| | STYRVVSVLTVLHQDWLNGKEYKCKVSNKAMPEPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 472) | |
| VFC-46 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGFSVFLFPPKPKDTLM | |
| | ISRTPEVTCVVVDVSHEDPEVKENWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 473) | |
| VFC-47 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGEFSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 474) | |
| VFC-48 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGDESVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 475) | |
| VFC-49 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGFSVFLFPPKPKDTLM | |
| | ISRTPEVTCVVVDVKHEDPEVKFNWYVDGVEVHNAKTKPREEQYN | |
| | STYRVVSVLTVLHQDWLNGKEYKCKVSNKAMPEPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 476) | |
| VFC-50 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGQSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 477) | |
| VFC-51 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGEQSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 478) | |
| VFC-52 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGDQSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 479) | |
| VFC-53 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGQSVFLFPPKPKDTLM | |
| | ISRTPEVTCVVVDVKHEDPEVKFNWYVDGVEVHNAKTKPREEQYN | |
| | STYRVVSVLTVLHQDWLNGKEYKCKVSNKAMPEPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 480) | |
| VFC-54 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGMSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 481) | |
| VFC-55 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGEMSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 482) | |
| VFC-56 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGDMSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 483) | |
| VFC-57 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGMSVFLFPPKPKDTLM | |
| | ISRTPEVTCVVVDVKHEDPEVKFNWYVDGVEVHNAKTKPREEQYN | |
| | STYRVVSVLTVLHQDWLNGKEYKCKVSNKAMPEPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 484) | |
| VFC-58 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPEGLLGGDSVFLFPPKPKDTL | |
| | MISRTPEVTCVVVDVKHEDPEVKFNWYVDGVEVHNAKTKPREEQY | |
| | NSTYRVVSVLTVLHQDWLNGKEYKCKVSNKAMPEPIEKTISKAKG | |
| | QPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEA | |
| | LHNHYTQKSLSLSPGK (SEQ ID NO: 485) | |
| VFC-59 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPEGLLGGDSVFLFPPKPKDTL | |
| | MISRTPEVTCVVVDVKHEDPEVKFNWYVDGVEVHNAKTKPREEQY | |
| | NSTYRVVSVLTVLHQDWLNGKEYKCKVSNKDMPEPIEKTISKAKG | |
| | QPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEA | |
| | LHNHYTQKSLSLSPGK (SEQ ID NO: 486) | |
| VFC-60 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPEGLLGHDSVFLFPPKPKDTL | |
| | MISRTPEVTCVVVDVKHEDPEVKFNWYVDGVEVHNAKTKPREEQY | |
| | NSTYRVVSVLTVLHQDWLNGKEYKCKVSNKAMPEPIEKTISKAKG | |
| | QPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEA | |
| | LHNHYTQKSLSLSPGK (SEQ ID NO: 487) | |
| VFC-61 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSQYDPEVKFNWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 488) | |
| VFC-62 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDASQYDPEVKFNWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 489) | |
| VFC-63 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVVDVSQYDPEVKFNWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 490) | |
| VFC-64 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSNYDPEVKFNWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 491) | |
| VFC-65 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVVDVSQYEPEVKFNWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 492) | |
| VFC-66 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVVDASQYEPEVKFNWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 493) | |
| VFC-67 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVVDVSNYEPEVKENWYVDGVEVHNAKTKPREEQNN | |
| | SFYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 494) | |
| VFC-68 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPEVFLFPPKPKDTLM | |
| | ISRTPEVTCVVVFVSQYEPEVKFNWYVDGVEVHNAKTKPREEQNN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 495) | |
| VFC-69 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALDAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 496) | |
| VFC-70 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSHEDPEVKENWYVDGVEVHNAKTKPREEQYN | |
| | STYRVVSVLTVLHQDWLNGKEYKCKVSNKDLDAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 497) | |
| VFC-71 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSDKALDAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 498) | |
| VFC-72 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSDKDLDAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 499) | |
| VFC-73 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSHEDPEVKENWYVDGVEVHNAKTKPREEQYN | |
| | STYRVVSVLTVLHQDWLNGKEYKCKVSNKALEAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 500) | |
| VFC-74 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSHEDPEVKFNWYVDGVEVHNAKTKPREEQYN | |
| | STYRVVSVLIVLHQDWLNGKEYKCKVSNKALRAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 501) | |
| VFC-75 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPEFEGGPSVFLFPPKPKDTLM | var_2_hole, L234F, |
| | ISRTPEVTCVVVDVSDEDGEVKFNWYVDGVEVHNAKTKPREEQYN | L235E, P329A, H268D, |
| | STYRVVSVLTVLHQDWLNGKEYKCKVSNKALAAPIEKTISKAKGQ | P271G, |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 502) | |
| VFC-76 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELLGGDSVFLFPPKPKDTLM | var_1_knob, P238D, |
| | ISRTPEVTCVVVDVSHDEPEVKENWYVDGVEVHNAKTKPREEQYN | E269D, D270E, A330R |
| | STYRVVSVLTVLHQDWLNGKEYKCKVSNKALPRPIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 503) | |
| VFC-77 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELVGGESVFLFPPKPKDTLM | var_7_knob, L235V, |
| | ISRTPEVTCVVVDVSHDEPEVKFNWYVDGVEVHNAKTKPREEQYN | P238E, E269D, D270E, |
| | STYRVVSVLTVLHQDWLNGKEYKCKVSNKALPRPIEKTISKAKGQ | A330R |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 504) | |
| VFC-78 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELLGGSSVFLFPPKPKDTLM | var_3_knob, P238S, |
| | ISRTPEVTCVVVDVSHDEPEVKENWYVDGVEVHNAKTKPREEQYN | E269D, D270E, A330R |
| | STYRVVSVLTVLHQDWLNGKEYKCKVSNKALPRPIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 505) | |
| VFC-79 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPEFEGGPSVFLFPPKPKDTLM | var_10_igG4_CH2_hole, |
| | ISRTPEVTCVVVDVSDEDGEVQFNWYVDGVEVHNAKTKPREEQFN | L234F, L235E, P329A, |
| | STYRVVSVLTVLHQDWLNGKEYKCKVSNKALASSIEKTISKAKGQ | H268D, P271G, |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 506) | |
| VFC-80 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELLGDGSVFLFPPKPKDTLM | var_4_knob, G237D, |
| | ISRTPEVTCVVVDVSHDEPEVKFNWYVDGVEVHNAKTKPREEQYN | P238G, E269D, D270E, |
| | STYRVVSVLTVLHQDWLNGKEYKCKVSNKALPRPIEKTISKAKGQ | A330R |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 507) | |
| VFC-81 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPEFEGGPDVFLFPPKPKDTLM | var_11_igG4_CH2_hole, |
| | ISRTPEVTCVVVDVSDEDGEVQFNWYVDGVEVHNAKTKPREEQFN | L234F, L235E, S239D, |
| | STYRVVSVLTVLHQDWLNGKEYKCKVSNKALASSIEKTISKAKGQ | P329A, H268D, P271G, |
| | PREPQVYTLPPSRDELTKNQVSLSCAVKGFYPSDIAVEWESNGQP | |
| | ENNYKTTPPVLDSDGSFFLVSKLTVDKSRWQQGNVFSCSVMHEAL | |
| | HNHYTQKSLSLSPGK (SEQ ID NO: 508) | |
| VFC-82 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELLGDSGVFLFPPKPKDTLM | var_5_knob, G237D, |
| | ISRTPEVTCVVVDVSHDEPEVKENWYVDGVEVHNAKTKPREEQYN | P238S, S239G, E269D, |
| | STYRVVSVLTVLHQDWLNGKEYKCKVSNKALPRPIEKTISKAKGQ | D270E, A330R |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 509) | |
| VFC-83 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPEFEGGPSVFLFPPKPKDTLM | var_9_igG4_ch2_knob, |
| | ISRTPEVTCVVVDVSQEDPEVQFNWYVDGVEVHNAKTKPREEQEN | L234F, L235E, |
| | STYRVVSVLTVLHQDWLNGKEYKCKVSNKALPSSIEKTISKAKGQ | |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 510) | |
| VFC-84 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELLGDGSAFLFPPKPKDTLM | var_6_knob, G237D, |
| | ISRTPEVTCVVVDVSHDEPEVKFNWYVDGVEVHNAKTKPREEQYN | P238G, V240A, E269D, |
| | STYRVVSVLTVLHQDWLNGKEYKCKVSNKALPRPIEKTISKAKGQ | D270E, A330R |
| | PREPQVYTLPPSRDELTKNQVSLWCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK (SEQ ID NO: 511) | |
| VFC-85 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPEGEVLVGGDSVFLFPPKPKD | var_8_knob, insertion |
| | TLMISRTPEVTCVVVDVSHDEPEVKFNWYVDGVEVHNAKTKPREE | GEV, L235V, P238D, |
| | QYNSTYRVVSVLTVLHQDWLNGKEYKCKVSNKALPRPIEKTISKA | E269D, D270E, A330R |
| | KGQPREPQVYTLPPSRDELTKNQVSLWCLVKGFYPSDIAVEWESN | |
| | GQPENNYKTTPPVLDSDGSFFLYSKLTVDKSRWQQGNVFSCSVMH | |
| | EALHNHYTQKSLSLSPGK (SEQ ID NO: 512) | |
| VFC-86 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELLGEESVFLFPPKPKDTLM | G237E, P238E |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPG (SEQ ID NO: 543) | |
| VFC-87 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELLGDGSAFLFPPKPKDTLM | G237D, P238G, V240A, |
| | ISRTPEVTCVVVDVSHDEPEVKFNWYVDGVEVHNAKTKPREEQYN | E269D, D270E, A330R |
| | STYRVVSVLTVLHQDWLNGKEYKCKVSNKALPRPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPG (SEQ ID NO: 544) | |
| VFC-88 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPEFEGGPSVFLFPPKPKDTLM | L234F, L235E, P329A, |
| | ISRTPEVTCVVVDVSDEDGEVKENWYVDGVEVHNAKTKPREEQYN | H268D, P271G |
| | STYRVVSVLTVLHQDWLNGKEYKCKVSNKALAAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPG (SEQ ID NO: 545) | |
| VFC-89 | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPG (SEQ ID NO: 546) | |
| VFC-90 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELLGEESVFLFPPKPKDTLM | P238E, G237E, M428L, |
| | ISRTPEVTCVVVDVSHEDPEVKFNWYVDGVEVHNAKTKPREEQYN | N434S |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVLHEAL | |
| | HSHYTQKSLSLSPG (SEQ ID NO: 683) | |
| VFC-91 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELLGEESVFLFPPKPKDTLY | P238D, G237E, M252Y, |
| | ITREPEVTCVVVDVSHEDPEVKFNWYVDGVEVHNAKTKPREEQYN | S254T, T256E |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPG (SEQ ID NO: 684) | |
| VFC-92 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELLGDGSAFLFPPKPKDTLM | G237D, P238G, V240A, |
| | ISRTPEVTCVVVDVSHDEPEVKFNWYVDGVEVHNAKTKPREEQYN | E269D, D270E, A330R, |
| | STYRVVSVLTVLHQDWLNGKEYKCKVSNKALPRPIEKTISKAKGQ | M428L, N434S |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVLHEAL | |
| | HSHYTQKSLSLSPG (SEQ ID NO: 685) | |
| VFC-93 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELLGDGSAFLFPPKPKDTLY | G237D, P238G, V240A, |
| | ITREPEVTCVVVDVSHDEPEVKFNWYVDGVEVHNAKTKPREEQYN | E269D, D270E, A330R, |
| | STYRVVSVLTVLHQDWLNGKEYKCKVSNKALPRPIEKTISKAKGQ | M252Y, S254T, T256E |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPG (SEQ ID NO: 686) | |
| VFC-94 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPEFEGGPSVFLFPPKPKDTLM | L234F, L235E, P329A, |
| | ISRTPEVTCVVVDVSDEDGEVKFNWYVDGVEVHNAKTKPREEQYN | H268D, P271G, M428L, |
| | STYRVVSVLTVLHQDWLNGKEYKCKVSNKALAAPIEKTISKAKGQ | N434S |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVLHEAL | |
| | HSHYTQKSLSLSPG (SEQ ID NO: 687) | |
| VFC-95 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPEFEGGPSVFLFPPKPKDTLY | L234F, L235E, P329A, |
| | ITREPEVTCVVVDVSDEDGEVKFNWYVDGVEVHNAKTKPREEQYN | H268D, P271G, M252Y, |
| | STYRVVSVLTVLHQDWLNGKEYKCKVSNKALAAPIEKTISKAKGQ | S254T, T256E |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPG (SEQ ID NO: 688) | |
| VFC-96 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELLGGDSVFLFPPKPKDTLM | P238D, M428L, N434S |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVLHEAL | |
| | HSHYTQKSLSLSPG (SEQ ID NO: 689) | |
| VFC-97 | ASTKGPSVFPLAPSSKSTSGGTAALGCLVKDYFPEPVTVSWNSGA | Mutations as compared |
| | LTSGVHTFPAVLQSSGLYSLSSVVTVPSSSLGTQTYICNVNHKPS | to SEQ ID NO: 516: |
| | NTKVDKKVEPKSCDKTHTCPPCPAPELLGGDSVFLFPPKPKDTLY | P238D, M252Y, S254T, |
| | ITREPEVTCVVVDVSHEDPEVKFNWYVDGVEVHNAKTKPREEQYN | T256E |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPG (SEQ ID NO: 690) | |

In some embodiments, an Fc polypeptide comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 417, 418, 419, 420, 421, 422, 423, 424, 425, 426, 427, 428, 429, 430, 431, 432, 434, 435, 436, 437, 438, 439, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 543, 544, 545, 546, 683, 684, 685, 686, 687, 688, 689, and 690, provided that the Fc polypeptide comprises the mutation or mutations for the reference sequence as set forth in the table above (Table 4) or that would be immediately apparent by aligning the reference sequence with SEQ ID NO: 516 using BLASTP or Clustal Omega with default settings.

In some embodiments, the Fc polypeptide comprises an amino acid sequence having at least 90-99% identity with an amino acid comprising SEQ ID NO: 516 or SEQ ID NO: 543, provided that the Fc polypeptide comprises the mutations of G237E and P238E, according to EU numbering.

In some embodiments, the Fc polypeptide comprises an amino acid sequence having at least 90-99% identity with an amino acid comprising SEQ ID NO: 516, provided that the Fc polypeptide comprises the mutations of P238D, according to EU numbering.

In some embodiments, the Fc polypeptide comprises what is referred to as the “AAA” mutations, which are Leu234Ala, Leu235Ala, and Gly237Ala (EU numbering).

In some embodiments, the Fc polypeptide comprises what is referred to as the “LALA” mutations, which are Leu234Ala and Leu235Ala (EU numbering).

In some embodiments, the Fc polypeptide comprises at least one mutation that extends the half-life of the Fc polypeptide. In some embodiments, the at least one mutation that extends the half-life of the Fc polypeptide, is such as those known in the art, such as, without limitation, a set of mutations of M428L and N434S (“LS” mutations), or M252Y, S254T, and T256E (“YTE” mutations) mutations. The extension mutations can be combined with or used independently of the other Fc mutations provided for herein, such as those that provide selective binding to FcgRIIB or those that impair the function of the Fc polypeptide or that make the Fc polypeptide effectorless, such as “AAA” or “LALA”.

In some embodiments, an Fc polypeptide comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 417, 418, 419, 420, 421, 422, 423, 424, 425, 426, 427, 428, 429, 431, 432, 433, 434, 435, 436, 437, 438, 439, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 543, 544, 545, 546, 683, 684, 685, 686, 687, 688, 689, and 690, and comprises a set of mutations of M428L and N434S. In some embodiments, an Fc polypeptide comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 417, 418, 419, 420, 421, 422, 423, 424, 425, 426, 427, 428, 429, 431, 432, 433, 434, 435, 436, 437, 438, 439, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 543, 544, 545, 546, 683, 684, 685, 686, 687, 688, 689, and 690, and comprises a set of mutations of M252Y, S254T, and T256E.

In some embodiments, the Fc polypeptide comprises an amino acid sequence that is at least at least 90%, at least 91%, at least 92%, at least 93%, at least 94%, at least 95%, at least 96%, at least 97%, at least 98%, or at least 99% identical to SEQ ID NO: 543, provided that the Fc comprises the mutations of G237E and P238E. In some embodiments, the Fc further comprises the YTE or LS mutations.

In some embodiments, the Fc polypeptide comprises an amino acid sequence that is at least at least 90%, at least 91%, at least 92%, at least 93%, at least 94%, at least 95%, at least 96%, at least 97%, at least 98%, or at least 99% identical to SEQ ID NO: 516, provided that the Fc comprises the mutations of P238D. In some embodiments, the Fc further comprises the YTE or LS mutations.

In some embodiments, an Fc polypeptide comprises an amino acid sequence selected from any one of SEQ ID NO: 417, 418, 419, 420, 421, 422, 423, 424, 425, 426, 427, 428, 429, 430, 431, 432, 434, 435, 436, 437, 438, 439, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 543, 544, 545, 546, 683, 684, 685, 686, 687, 688, 689, and 690.

In some embodiments, the Fc polypeptide comprises mutations that render the Fc polypeptide “effectorless” and unable to bind Fc receptors. The mutations that render Fc polypeptides effectorless are known in the art and any mutation or combination of mutations can be used, such as AAA and LALA (provided for herein).

In some embodiments, the mutations in the Fc polypeptide, which is according to the known numbering system, are selected from the group consisting of: L234A, L235A, L234F, L235E, P329G, P331S, N297A, N297G, N297Q, G236A, A330S, S239D, 1332E, S267E, H268F, S324T, Y296W, T299A, V308P, H310A, R409K, Y435H, T307A, T309A, T309K, K322A, K326W, K334W, K326A, K334A, G237A, P238S, H268A, or any combination thereof. In some embodiments, the Fc comprises a mutation at L234 and/or L235 and/or G237. In some embodiments, the Fc comprises L234A and/or L235A mutations, which can be referred to as “LALA” mutations. In some embodiments, the Fc comprises L234A, L235A, and G237A mutations, which can be referred to as “LALAGA” or “AAA”. In some embodiments, the Fc comprises L234F, and L235E mutations, which can be referred to as “LAFE” mutations. In some embodiments, the Fc comprises L234A, L235A, and P329G mutations, which can be referred to as “LALAPG” mutations. In some embodiments, the Fc comprises L234A, L235A, P329G, and P331S mutations, which can be referred to as “LALAPGS” mutations. In some embodiments, the Fc comprises L234A, L235A, and P329S mutations, which can be referred to as “LALAPS” mutations. In some embodiments, the Fc comprises a N297A mutation. In some embodiments, the Fc mutations comprises a N297G mutation. In some embodiments, the Fc comprises a N297Q mutation. In some embodiments, the Fc comprises a P329G mutation. In some embodiments, the Fc comprises G236A, A330S, and P331S mutations, which can be referred to as “GASDALIE” mutations. In some embodiments, the Fc comprises S239D and I332E mutations, which can be referred to as “SIE” mutations. In some embodiments, the Fc comprises S267E, H268F, S324T, and I332E mutations, which can be referred to as “SEHF_STIE” mutations. In some embodiments, the Fc comprises Y296W, T299A, and V308P mutations, which can be referred to as “YTEV” mutations. In some embodiments, the Fc comprises H310A, R409K, and Y435H mutations, which can be referred to as “HRY” mutations. In some embodiments, the Fc comprises T307A and T309A mutations, which can be referred to as “TATA” mutations. In some embodiments, the Fc comprises T307A and T309K mutations, which can be referred to as “TAKA” mutations. In some embodiments, the Fc comprises a K322A mutation. In some embodiments, the Fc comprises K326W and K334W mutations, which can be referred to as “WKWK” mutations. In some embodiments, the Fc comprises K326A and K334A mutations, which can be referred to as “AA” mutations. In some embodiments, the Fc comprises L234A, L235A, G237A, P238S, H268A, A330S, and P331S mutations.

The mutations and positions of the Fc polypeptide, which can also be referred to as the Fc polypeptide, are according to EU numbering.

As used herein, a Fc polypeptide/domain comprising a mutation at a specific position is as compared to the wild-type Fc according the numbering system (EU numbering) as referenced herein.

In some embodiments, the Fc polypeptide sequence comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to:

(SEQ ID NO: 513)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGAPSVFLFPPKPKDTLMISRTPEVTCVVVDVS
HEDPEVKENWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG.

In some embodiments, the Fc polypeptide comprises an amino acid sequence of SEQ ID NO: 513.

In some embodiments, the Fc polypeptide is linked to the inhibitory receptor effector domain. In some embodiments, the Fc polypeptide is linked to a C-terminus of the inhibitory receptor effector domain. In some embodiments, when the inhibitory receptor effector domain is an antibody, the Fc polypeptide is linked to the C-terminus of the heavy chain of the antibody that forms the inhibitor receptor effector domain. In some embodiments, the N-terminus of the Fc polypeptide is linked to the C-terminus of the inhibitory receptor effector domain. In some embodiments, the Fc polypeptide is directly linked, such as without a linker sequence, to the inhibitory receptor effector domain. In some embodiments, the Fc polypeptide is linked to the inhibitory receptor effector domain through a linker, such as a peptide linker. In some embodiments, the linker is as provided for herein.

Examples of peptide linkers that can be used are known in the art and non-limiting examples are provide for herein.

As used herein, the term “FcγRII binding effector domain” refers to a polypeptide, such as an antibody, that binds to FcγRII receptor. Examples of such receptors include the FcγRIIα or FcγRIIb receptor. In some embodiments, the FcγRII binding effector domain is an antibody. In some embodiments, the FcγRII binding effector domain is a scFv antibody. In some embodiments, the N-terminus of the FcγRII binding effector domain is bound to the C-terminus of the Fc polypeptide. In some embodiments, the FcγRII binding effector domain selectively binds to the FcγRIIb receptor. In some embodiments, the FcγRII binding effector domain selectively binds to the FcγRIIb receptor over the FcγRIIα receptor.

Antibody molecule, as that term is used herein, refers to a polypeptide, e.g., an immunoglobulin chain or fragment thereof, comprising at least one functional immunoglobulin variable domain sequence. An antibody molecule encompasses antibodies (e.g., full-length antibodies) and antibody fragments. In some embodiments, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, comprises a portion of an antibody, e.g., Fab, Fab′, F(ab′) 2, F (ab) 2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′) 2 fragments, and single chain variable fragments (scFvs).

The term “antibody molecule” also encompasses whole or antigen binding fragments of domain, or single domain, antibodies, which can also be referred to as “sdAb” or “VHH.” Domain antibodies comprise either V_{H} or V_{L} that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91, which is hereby incorporated by reference). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in WO2004041862 and WO2016065323, each of which is hereby incorporated by reference. The domain antibodies can be a targeting moiety as described herein.

Antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, hexavalent), or with higher orders of specificity (e.g, tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide. Effector, as that term is used herein, refers to an entity, e.g., a cell or molecule, e.g., a soluble or cell surface molecule, which mediates an immune response. In some embodiments, the effector is an antibody. In some embodiments, the effectors binding domains as provided for herein, refers to a polypeptide (e.g.) that has sufficient binding specificity that it can bind the effector with sufficient specificity that it can serve as an effector binding/modulating molecule. In some embodiments, it binds to effector with at least 10, 20, 30, 40, 50, 60, 70, 80, 90, or 95% of the affinity of the naturally occurring counter-ligand. In some embodiments, it has at least 60, 70, 80, 90, 95, 99, or 100% sequence identity, or substantial sequence identity, with a naturally occurring counter-ligand for the effector.

Elevated risk, as used herein, refers to the risk of a disorder in a subject, wherein the subject has one or more of a medical history of the disorder or a symptom of the disorder, a biomarker associated with the disorder or a symptom of the disorder, or a family history of the disorder or a symptom of the disorder.

In some embodiments, the inhibitory effector binding domain can be referred to as an inhibitory immune checkpoint molecule. This can refer to a polypeptide that can bind to the checkpoint molecule and agonize its cognate inhibitory activity. For example, the antibody can be an anti-PD-1 antibody, or an antigen-binding fragment thereof, that binds to PD-1 and agonizes PD-1's activity. In some embodiments, the antibody inhibits the inhibitory checkpoint activity, such that it antagonizes the inhibitory activity. For example, the antibody can be an anti-PD-1 antibody, or an antigen-binding fragment thereof, that binds to PD-1 and antagonizes PD-1's activity. The same can be done if the target is any of the inhibitory receptors, such as those provided for herein. In some embodiments, the inhibitory checkpoint receptor is LAG-3. In some embodiments, the inhibitory checkpoint receptor is as provided for herein. These are non-limiting examples and other inhibitory checkpoint receptors can be agonized or antagonized as provided for herein.

Inhibitory receptor agonism can be elicited either by engagement of the natural ligand of the inhibitory receptor or via antibody crosslinking and higher order clustering of the inhibitory receptors. Thus, immune homeostasis may be restored by agonizing multiple inhibitory receptors (IRs) with one antibody. Without wishing to be bound by a particular theory, agonism of IRs may modulate the network interactions of multiple pathologic immune cell types, thus restoring immune homeostasis in diseases of cell-mediated immunity. Agonizing inhibitory receptors with an antibody molecules can require IR superclustering on the surface of the cell, which is not efficiently induced by Fc-null antibodies. For example, Programmed cell death 1 (PD-1) is a negative costimulatory receptor essential for suppression of T cell activation both in in vitro and in vivo. Studies show that upon interacting with its ligand, PD-L1, PD-1 forms clusters with T cell receptors (TCRs) and transiently associates with the phosphatase SHP2 (Src homology 2 domain-containing tyrosine phosphatase. These inhibitory microclusters trigger the dephosphorylation of nearby TCR signaling molecules, resulting in suppression of T cell activation (Yokosuka T, Takamatsu M, Kobayashi-Imanishi W, Hashimoto-Tane A, Azuma M, Saito T. Programmed cell death 1 forms negative costimulatory microclusters that directly inhibit T cell receptor signaling by recruiting phosphatase SHP2. J Exp Med. 2012 Jun. 4; 209 (6): 1201-17. doi: 10.1084/jem.20112741. Epub 2012 May 28. PMID: 22641383; PMCID: PMC3371732.). Thus, agonist antibody molecules may rely on simultaneous Fc (constant region) tethering on antigen presenting cells (APC), thereby allowing efficient IR superclustering and downstream signaling. Agonistic molecules targeting IRs and containing an IgG1 wild-type Fc bind both activating and inhibitory Fc receptors, thus triggering unwanted production of inflammatory cytokines by APCs as a result of binding the activating Fc receptors. However, selectively binding to FcγRIIβ (the only inhibitory Fc receptor) prevents proinflammatory cytokine production and may also inhibit pathogenic B cell and APC activity.

Accordingly, in some embodiments, provided herein are variant Fc polypeptides comprising a mutation, or set of mutations, that increase selectivity for an FcγRIIβ receptor. In some embodiments, provided herein are a dimer molecule comprising variant Fc polypeptides comprising a mutation, or set of mutations, that increase selectivity for an FcγRIIβ receptor. In some embodiments, the dimer molecule comprising variant Fc polypeptides comprising a mutation, or set of mutations, that increase selectivity for FcγRIIβ, binds to one FcγRIIβ receptor. In some embodiments, the dimer molecule comprises a first variant Fc polypeptide comprising a mutation, or set of mutations, that increase selectivity for FcγRIIβ, and a second variant Fc polypeptide comprising a mutation, or set of mutations, that increase selectivity for FcγRIIβ. In some embodiments, the first variant Fc polypeptide and the second variant Fc polypeptide are the same, such that it is a homodimer in respect to the variant Fc polypeptide. In some embodiments, the first variant Fc polypeptide and the second variant Fc polypeptide are different, such that it is a heterodimer in respect to the variant Fc polypeptide. In some embodiments, the first variant Fc polypeptide comprising a mutation, or set of mutations, that increase selectivity for FcγRIIβ, and the second variant Fc polypeptide comprising a mutation, or set of mutations, that increase selectivity for FcγRIIβ, both bind to the same FcγRIIβ receptor. In some embodiments, a mutation, or set of mutations, that increase selectivity for FcγRIIβ may also affect binding of the antibody to the IR. In some embodiments, a mutation, or set of mutations, that increase selectivity for FcγRIIβ may also increase binding of the antibody to the IR. In some embodiments, a mutation, or set of mutations, that increase selectivity for FcγRIIβ may also decrease binding of the antibody to the IR. In some embodiments, the variant Fc polypeptide comprising a mutation, or set of mutations, that increase selectivity for FcγRIIβ is conjugated or linked to an antibody. In some embodiments, the variant Fc polypeptide comprising a mutation, or set of mutations, that increase selectivity for FcγRIIβ is conjugated or linked to an agonistic antibody. In some embodiments, the first variant Fc polypeptide comprising a mutation, or set of mutations, that increase selectivity for FcγRIIβ is conjugated or linked to an antibody. In some embodiments, the first variant Fc polypeptide comprising a mutation, or set of mutations, that increase selectivity for FcγRIIβ is conjugated or linked to an agonistic antibody. In some embodiments, the second variant Fc polypeptide comprising a mutation, or set of mutations, that increase selectivity for FcγRIIβ is conjugated or linked to an antibody. In some embodiments, the second variant Fc polypeptide comprising a mutation, or set of mutations, that increase selectivity for FcγRIIβ is conjugated or linked to an agonistic antibody. In some embodiments, the first variant Fc polypeptide and the second variant Fc polypeptide, each comprising a mutation, or set of mutations, that increase selectivity for FcγRIIβ is each conjugated or linked to an antibody. In some embodiments, the first variant Fc polypeptide and the second variant Fc polypeptide, each comprising a mutation, or set of mutations, that increase selectivity for FcγRIIβ is each conjugated or linked to an agonistic antibody. In some embodiments, the antibody binds to an IR. In some embodiments, the agonistic antibody binds to an IR. In some embodiments, a mutation, or set of mutations, that increase selectivity for FcγRIIβ may also affect binding of the antibody to the IR. In some embodiments, a mutation, or set of mutations, that increase selectivity for FcγRIIβ may also increase binding of the antibody to the IR. In some embodiments, a mutation, or set of mutations, that increase selectivity for FcγRIIβ may also decrease binding of the antibody to the IR. In some embodiments, a mutation, or set of mutations, that increase selectivity for FcγRIIβ may affect clustering of PD-1. In some embodiments, a mutation, or set of mutations, that increase selectivity for FcγRIIβ may increase clustering of PD-1. In some embodiments, a mutation, or set of mutations, that increase selectivity for FcγRIIβ may decrease clustering of PD-1. In some embodiments, a mutation, or set of mutations, that increase selectivity for FcγRIIβ may affect clustering of PD-1, wherein affecting clustering of PD-1 also affects agonism of PD-1. In some embodiments, a mutation, or set of mutations, that increase selectivity for FcγRIIβ may increase clustering of PD-1, wherein increasing clustering of PD-1 also increases agonism of PD-1. In some embodiments, a mutation, or set of mutations, that increase selectivity for FcγRIIβ may decrease clustering of PD-1, wherein decrease clustering of PD-1 also decrease agonism of PD-1.

The domains can have similarity to those as provided for herein or those that are incorporated by reference. Sequence identity, percentage identity, and related terms, as those terms are used herein, refer to the relatedness of two sequences, e.g., two nucleic acid sequences or two amino acid or polypeptide sequences. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, such as those encoding for the domains, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence. For example, a Fc variant can have the sequence of a Fc domain but comprise a mutation that affects its binding to the FcγRIIα or FcγRIIb receptor. In some embodiments, the Fc variant selectively binds to the FcγRIIb receptor. In some embodiments, the Fc variant selectively binds to the FcγRIIb receptor over the FcγRIIα receptor.

Calculations of homology or sequence identity between sequences (the terms are used interchangeably herein) can be performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to for example any a nucleic acid sequence provided herein. BLAST protein searches can be performed with the XBLAST program, score=50, wordlength=3 to obtain amino acid sequences homologous to protein molecules provided herein. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules and compounds of the present embodiments may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The present disclosure provides, for example, effector domains that can act as PD-1 agonists. Without being bound to any particular theory, agonism of PD-1 inhibits T cell activation/signaling and can be accomplished by different mechanisms. For example cross-linking can lead to agonism, bead-bound, functional PD-1 agonists have been described (Akkaya. Ph.D. Thesis: Modulation of the PD-1 pathway by inhibitory antibody superagonists. Christ Church College, Oxford, U K, 2012), which is hereby incorporated by reference. Crosslinking of PD-1 with two mAbs that bind non-overlapping epitopes induces PD-1 signaling (Davis, US 2011/0171220), which is hereby incorporated by reference. Another example is illustrated through the use of a goat anti-PD-1 antiserum (e.g. AF1086, R&D Systems) which is hereby incorporated by reference, which acts as an agonist when soluble (Said et al., 2010, Nat Med) which is hereby incorporated by reference. Non-limiting examples of PD-1 agonists that can be used in the present embodiments include, but are not limited to, UCB clone 19 or clone 10, PD1AB-1, PD1AB-2, PD1AB-3, PD1AB-4 and PD1AB-5, PD1AB-6 (Anaptys/Celgene), PD1-17, PD1-28, PD1-33 and PD1-35 (Collins et al, US 2008/0311117 A1).

Antibodies against PD-1 and uses therefor, which is incorporated by reference), or can be a bi-specific, monovalent anti-PD-1/anti-CD3 (Ono), and the like. In some embodiments, the PD-1 agonist antibodies can be antibodies that block binding of PD-L1 to PD-1. In some embodiments, the PD-1 agonist antibodies can be antibodies that do not block binding of PD-L1 to PD-1.

PD-1 agonism can be measured by any method, such as the methods described in the examples. For example, cells can be constructed that express, including stably express, constructs that include a human PD-1 polypeptide fused to a b-galactosidase “Enzyme donor” and 2) a SHP-2 polypeptide fused to a b-galactosidase “Enzyme acceptor.” Without being bound by any theory, when PD-1 is engaged, SHP-2 is recruited to PD-1. The enzyme acceptor and enzyme donor form a fully active b-galactosidase enzyme that can be assayed. Although, the assay does not directly show PD-1 agonism, but shows activation of PD-1 signaling. PD-1 agonism can also be measured by measuring inhibition of T cell activation because, without being bound to any theory, PD-1 agonism inhibits anti-CD3-induced T cell activation. For example, PD-1 agonism can be measured by preactivating T cells with PHA (for human T cells) or ConA (for mouse T cells) so that they express PD-1. The cells can then be reactivated with anti-CD3 in the presence of anti-PD-1 (or PD-L1) for the PD-1 agonism assay. T cells that receive a PD-1 agonist signal in the presence of anti-CD3 will show decreased activation, relative to anti-CD3 stimulation alone. Activation can be readout by proliferation or cytokine production (IL-2, IFNγ, IL-17) or other markers, such as CD69 activation marker. Thus, PD-1 agonism can be measured by either cytokine production or cell proliferation. Other methods can also be used to measure PD-1 agonism.

In some embodiments, PD-1 agonism is increased when a PD-1 agonist is linked to an effector domain or other binding moiety that binds specifically to FcγRIIb. In some embodiments, PD-1 agonism is increased when a PD-1 agonist is linked to an effector moiety that selectively binds to FcγRIIb. In some embodiments, the effector is an antibody that selectively binds to FcγRIIb. In some embodiments, PD-1 agonism is increased when a PD-1 agonist is linked to an Fc polypeptide that selectively binds to FcγRIIb. In some embodiments, the antibody is in an scFv format. In some embodiments, the PD-1 agonist is linked to both an Fc polypeptide and an antibody that each selectively binds to FcγRIIb. In some embodiments, only one of the Fc polypeptide and the antibody selectively binds to FcγRIIb. In some embodiments, the Fc polypeptide is effectorless. In some embodiments, the PD-1 agonist is an antibody that binds to PD-1. In some embodiments, the PD-1 agonist is an anti-PD-1 antibody, or an antigen-binding fragment thereof. In some embodiments, the antibody has little or no antagonist activity against PD-1. In some embodiments, The anti-PD-1 antibody, or the antigen-binding fragment thereof, is in a Fab format. In some embodiments, The anti-PD-1 antibody, or the antigen-binding fragment thereof, is in a scFv format.

PD-1 is an Ig superfamily member expressed on activated T cells and other immune cells. The natural ligands for PD-1 appear to be PD-L1 and PD-L2. Without being bound to any particular theory, when PD-L1 or PD-L2 bind to PD-1 on an activated T cell, an inhibitory signaling cascade is initiated, resulting in attenuation of the activated T effector cell function. Thus, blocking the interaction between PD-1 on a T cell, and PD-L1/2 on another cell (eg tumor cell) with a PD-1 antagonist is known as checkpoint inhibition, and releases the T cells from inhibition. In contrast, PD-1 agonist antibodies can bind to PD-1 and send an inhibitory signal and attenuate the function of a T cell. Thus, PD-1 agonist antibodies can be incorporated into various embodiments described herein as an effector molecule binding/modulating moiety, which can accomplish localized tissue-specific immunomodulation when paired with a targeting moiety.

In some embodiments, the antibody is an anti-PD-1 antibody which binds to PD-1. In some embodiments, the antibody binds to amino acids of an epitope of PD-1. The epitopes are described herein, such as in the Tables and described in the Examples.

In some embodiments, anti-PD-1 antibodies, such as those provided herein, bind to an epitope on PD-1. PD-1 is a type I membrane protein, which has the amino acid sequence as set forth in SEQ ID NO: 589:

(SEQ ID NO: 589)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

In some embodiments, anti-PD-1 antibodies, such as those provided herein, bind to an epitope on PD-1.

In some embodiments, anti-PD-1 antibodies, such as those provided herein, bind to an epitope on PD-1 that include PD-1 (SEQ ID NO 589) residues P39, A40, L41, L42, V43, V44, T45, D48, E61, S62, H107, L128, A129, P130, K131, A132, Q133, R143, T145, E146, R147, R148, A149, E150, V151, P152, A154, or any combination thereof. In some embodiments, the epitope includes residues P39, A40, L41, L42, V43, V44, T45, D48, H107, R143, T145, R147, and R148 of PD-1. In some embodiments, the epitope includes residues E61, S62, L128, A129, P130, K131, A132, and Q133 of PD-1. In some embodiments, the epitope includes residues E146, R147, R148, A149, E150, V151, P152, and A154 of PD-1.

Without wishing to be bound by a particular theory, the PD-1 receptor may trigger a negative immunoregulatory mechanism that prevents overactivation of immune cells and subsequent inflammatory diseases. Thus, while immunoenhancing anti-PD-1 blocking antibodies have become a widely used cancer treatment, little is known about the required characteristics for anti-PD-1 antibodies to be capable of stimulating immunosuppressive activity.

In some embodiments, a PD-1 agonist antibody binds to a membrane proximal epitope on PD-1. As used herein, a “membrane proximal” epitope is an epitope on PD-1 protein structure that is in proximity to the cell membrane. In some embodiments, the membrane proximal epitope includes residues P39, A40, L41, L42, V43, V44, T45, D48, H107, R143, T145, R147, and R148 of PD-1. In some embodiments, the membrane proximal epitope includes residues E146, R147, R148, A149, E150, V151, P152, and A154 of PD-1.

In some embodiments, the antibody binds to the epitope, such as those provided for herein and above, with a low affinity. In some embodiments, the antibody that binds to PD-1 with low affinity binds to PD-1 with a binding affinity no greater than 50 nM. For example, an antibody that binds to PD-1 with an antibody of 10 nM would be understood to bind to PD-1 with a greater affinity than antibody that binds to PD-1 with an affinity of 50 nM. In some embodiments, the antibody that binds to PD-1 with low affinity binds to PD-1 with a binding affinity no greater than 70 nM. In some embodiments, the antibody that binds to PD-1 with low affinity binds to PD-1 with a binding affinity no greater than 80 nM. In some embodiments, the antibody that binds to PD-1 with low affinity binds to PD-1 with a binding affinity no greater than 90 nM. In some embodiments, the antibody that binds to PD-1 with low affinity binds to PD-1 with a binding affinity no greater than 100 nM. In some embodiments, the antibody that binds to PD-1 with low affinity binds to PD-1 with a binding affinity no greater than 110 nM. In some embodiments, the antibody that binds to PD-1 with low affinity binds to PD-1 with a binding affinity no greater than 120 nM. In some embodiments, the antibody that binds to PD-1 with low affinity binds to PD-1 with a binding affinity no greater than 130 nM. In some embodiments, the antibody that binds to PD-1 with low affinity binds to PD-1 with a binding affinity no greater than 140 nM. In some embodiments, the antibody that binds to PD-1 with low affinity binds to PD-1 with a binding affinity no greater than 150 nM. In some embodiments, the antibody that binds to PD-1 with low affinity binds to PD-1 with a binding affinity no greater than 152 nM. In some embodiments, the antibody that binds to PD-1 with low affinity binds to PD-1 with a binding affinity no greater than 258 nM. In some embodiments, the antibody that binds to PD-1 with low affinity binds to PD-1 with a binding affinity from about 50 nM to about 500 nM. In some embodiments, the antibody that binds to PD-1 with low affinity binds to PD-1 with a binding affinity from about 100 nM to about 500 nm. In some embodiments, the antibody that binds to PD-1 with low affinity binds to PD-1 with a binding affinity from about 200 nM to about 500 nM. In some embodiments, the antibody that binds to PD-1 with low affinity binds to PD-1 with a binding affinity from about 300 nM to about 500 nM. In some embodiments, the antibody that binds to PD-1 with low affinity binds to PD-1 with a binding affinity from about 400 nM to about 500 nM. In some embodiments, the antibody that binds to PD-1 with low affinity binds to PD-1 with a binding affinity from about 100 nM to about 400 nM. In some embodiments, the antibody that binds to PD-1 with low affinity binds to PD-1 with a binding affinity from about 100 nM to about 300 nM. In some embodiments, the antibody that binds to PD-1 with low affinity binds to PD-1 with a binding affinity from about 100 nM to about 200 nM. In some embodiments, the antibody that binds to PD-1 with low affinity binds to PD-1 with a binding affinity from about 200 nM to about 500 nM. In some embodiments, the antibody that binds to PD-1 with low affinity binds to PD-1 with a binding affinity from about 200 nM to about 400 nM. In some embodiments, the antibody that binds to PD-1 with low affinity binds to PD-1 with a binding affinity from about 200 nM to about 300 nM. In some embodiments, the antibodies referenced herein bind to the epitopes of PD-1, such as those provided herein, at affinities such as those provided herein. The affinity may be measured by any assay, such as those provided for in Example 43. In some embodiments, the affinity to PD-1 is measured using biolayer inferometry. In some embodiments, the measurement of antibody's affinity to PD-1 comprises the step of diluting an anti-PD-1 antibody is in assay buffer to a final concentration of 5 μg/mL, and titrating a recombinant human PD-1. In some embodiments, the measurement of antibody's affinity to PD-1 comprises the step of capturing the anti-PD-1 antibody on anti-human IgG Fc biosensors. In some embodiments, the measurement of antibody's affinity to PD-1 comprises the step of associating the anti-PD-1 antibody in wells with human PD-1, and dissociating in wells with assay buffer. In some embodiments, the measurement of antibody's affinity to PD-1 comprises the step of calculating kinetic parameters (kon and kdis) and equilibrium dissociation constant (KD) from a 1:1 Langmuir global Rmax fit model using the data analysis software of the Octet RED96 version 10.0.

Other examples of PD-1 antibodies that can be used include, but are not limited to, those described in JP6278224B2, JP2018518540A, CN1753912B, JP6174321B2, US20200190187A1, U.S. Pat. No. 10,676,516B2, WO2011082400A2, JP2017537090A, JP2012501670A, US2019/0270818, or CC-9000, each of which is hereby incorporated by reference in its entirety.

In some embodiments, the PD-1 antibody, or an antigen-binding fragment thereof, or antigen-binding fragment thereof, comprises a variable heavy chain amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% to:

(SEQ ID NO: 514)
QVQLVQSGAEVKKPGASVKVSCKVSGYSLSKYDMSWVRQAPGKGLEWMGI
IYTSGYTDYAQKFQGRVTMTEDTSTDTAYMELSSLRSEDTAVYYCATGNP
YYINGENSWGQGTLVTVSS.

In some embodiments, the PD-1 antibody, or an antigen-binding fragment thereof, or antigen-binding fragment thereof, comprises a variable heavy chain amino acid sequence of SEQ ID NO: 514.

In some embodiments, the PD-1 antibody, or an antigen-binding fragment thereof, or antigen-binding fragment thereof, comprises a variable light chain amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% to:

(SEQ ID NO: 515)
DIQMTQSPSSLSASVGDRVTITCQASQSPNNLLAWYQQKPGKAPKLLIYG
ASDLPSGVPSRFSGSGSGTDFTLTISSLQPEDFATYYCQNNYYVGPVSYA
FGGGTKVEIK.

In some embodiments, the PD-1 antibody, or an antigen-binding fragment thereof, or antigen-binding fragment thereof, comprises a variable light chain amino acid sequence of SEQ ID NO: 515.

In some embodiments, the PD-1 antibody, or an antigen-binding fragment thereof, or antigen-binding fragment thereof, comprises a variable heavy chain amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% to SEQ ID NO: 514; and a variable light chain amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% to SEQ ID NO: 515.

In some embodiments, the PD-1 antibody, or an antigen-binding fragment thereof, or antigen-binding fragment thereof, comprises a variable heavy amino acid sequence of SEQ ID NO: 514; and a variable light chain amino acid sequence of SEQ ID NO: 515.

In some embodiments, The anti-PD-1 antibody, or the antigen-binding fragment thereof, comprises a polypeptide comprising an amino acid sequence comprising any one variable heavy chains and any one variable light chains, or as combined with one another as provided in Table 5 below.

TABLE 5

| ID | VH | VL |
|---|---|---|
| PDAB | EVQLLESGGGLVQPGGSLRLSCAASGFTFSDYWMSWV | EIVMTQSPATLSVSPGERATLSCRASQSVSSNLAWYQQK |
| 1 | RQAPGKGLEWVSSIYSGTTYIYYADSVKGRFTISRDN | PGQAPRLLIYGASTRATGIPARFSGSGSGTEFTLTISSL |
| | SENTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 130) | NO: 323) |
| PDAB | EVQLLESGGGLVQPGGSLRLSCAASGFTFSDHYMSWV | EIVMTQSPATLSVSPGERATLSCRASQSVSSNLAWYQQK |
| 2 | RQAPGRGLEWVSTISSGGSYIYYADSVKGRFTISRDN | PGQAPRLLIYGASTRATGIPARFSGSGSGTEFTLTISSL |
| | SKNTLYLQMNSLRAEDTAVYYCAKILKNGNYIYYFDY | QSEDFAVYYCQQYNNWPPWTFGQGTKVEIK (SEQ ID |
| | WGQGTLVTVSS (SEQ ID NO: 131) | NO: 323) |
| PDAB | EVQLLESGGGLVQPGGSLRLSCAASGFTFTGYDMTWV | EIVMTQSPATLSVSPGERATLSCRASQSVSSNLAWYQQK |
| 3 | RQAPGKGLEWVSAISWNTGSTTYYADSVKGRFTISRD | PGQAPRLLIYGASTRATGIPARFSGSGSGTEFTLTISSL |
| | NSKNTLYLQMNSLRAEDTAVYYCTRGYDRKNYFEYWG | QSEDFAVYYCQQYNNWPPWTFGQGTKVEIK (SEQ ID |
| | QGTLVTVSS (SEQ ID NO: 132) | NO: 323) |
| PDAB | EVQLLESGGGLVQPGGSLRLSCAASGFTFSDHYMSWV | EIVMTQSPATLSVSPGERATLSCRASQSVSSNLAWYQQK |
| 4 | RQAPGKGLEWVSTISSGGSYIYYADSVKGRFTISRDN | PGQAPRLLIYGASTRATGIPARFSGSGSGTEFTLTISSL |
| | SKNTLYLQMNSLRAEDTAVYYCAKILKNGNYIYYFDY | QSEDFAVYYCQQYNNWPPWTFGQGTKVEIK (SEQ ID |
| | WGQGTLVTVSS (SEQ ID NO: 133) | NO: 323) |
| PDAB | EVQLLESGGGLVQPGGSLRLSCVASGFTEDDYGMTWV | EIVMTQSPATLSVSPGERATLSCRASQSVSSNLAWYQQK |
| 5 | RQASGKGLEFVATVNWNGNKTYYADSMKGRFTISRNN | PGQAPRLLIYGASTRATGIPARFSGSGSGTEFTLTISSL |
| | SENTVYLEVNSLRDEDTAIYYCVKNHEWKFEYWGQGT | QSEDFAVYYCQQYNNWPPWTFGQGIKVEIK (SEQ ID |
| | LVTVSS (SEQ ID NO: 134) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 6 | RQAPGKGLEWVSSIYSGPSYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 135) | NO: 323) |
| PDAB | EVQLLESGGGLVQPGGSLRLSCAASGFTFSSDAMNWV | EIVMTQSPATLSVSPGERATLSCRASQSVSSNLAWYQQK |
| 7 | RQAPGKGLEWVSTIYSGGSTYYADSVKGRFTISRDNS | PGQAPRLLIYGASTRATGIPARFSGSGSGTEFTLTISSL |
| | KNTLYLQMNSLRAEDTAVYYCARGGNFYNYFDYWGQG | QSEDFAVYYCQQYNNWPPWTFGQGTKVEIK (SEQ ID |
| | TLVTVSS (SEQ ID NO: 136) | NO: 323) |
| PDAB | EVQLLESGGGLVQPGGSLRLSCAASGFTFSDHYMSWV | EIVMTQSPATLSVSPGERATLSCRASQSVSSNLAWYQQK |
| 8 | RQAPGKGLEWVSTISSGGSYVYYADSVKGRFTISRDN | PGQAPRLLIYGASTRATGIPARFSGSGSGTEFTLTISSL |
| | SKNTLYLQMNSLRVEDAAVYYCAKILKNGKYIYYFDY | QSEDFAVYYCQQYNNWPPWTFGQGIKVEIK (SEQ ID |
| | WGQGTLVTVSS (SEQ ID NO: 137) | NO: 323) |
| PDAB | EVQLLESGGGLVQPGGSLRLSCAASGFTFSDHYMSWV | EIVMTQSPATLSVSPGERATLSCRASQSVSSNLAWYQQK |
| 9 | RQAPGKGLEWVSTISSGGSYKYYADSAKGRFTISRDN | PGQAPRLLIYGASTRATGIPARFSGSGSGTEFTLTISSL |
| | SKNTLYLQMNSLRAEDTAVYYCAKILKNGNYIYYFDY | QSEDFAVYYCQQYNNWPPWTFGQGTKVEIK (SEQ ID |
| | WGQGTLVTVSS (SEQ ID NO: 138) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 10 | RQAPGKGLEWVSSIYSGTSYIYYADSVKGRFTISRDN | PGQAPRLLIYGASTRATGIPARFSGSGSGTEFTLTISSL |
| | SRNTLYLQMNSLRAEDTAVYYCTRGWTYLDYWGQGTL | QSEDFAVYYCQQYNNWPPWTFGQGTKVEIK (SEQ ID |
| | VTVSS (SEQ ID NO: 139) | NO: 323) |
| PDAB | EVQLLESGGGLVQPGGSLRLSCAASGFTFSDHYMSWV | EIVMTQSPATLSVSPGERATLSCRASQSVSSNLAWYQQK |
| 11 | RQAPGKGLEWVSTISSGGSYVYYADSVKGRFTISRDN | PGQAPRLLIYGASTRATGIPARFSGSGSGTEFTLTISSL |
| | SKNTLHLQMNSLRVEDAAVYYCAKILKNGKYIYYFDY | QSEDFAVYYCQQYNNWPPWTFGQGTKVEIK (SEQ ID |
| | WGQGTLVTVSS (SEQ ID NO: 140) | NO: 323) |
| PDAB | EVQLLESGGGLVQPGGSLRLSCAAFGFTFTGYDMTWV | EIVMTQSPATLSVSPGERATLSCRASQSVSSNLAWYQQK |
| 12 | RQAPGKGLEWVSAISWNTGSTTYYADSVKGRFTISRD | PGQAPRLLIYGASTRATGIPARFSGSGSGTEFTLTISSL |
| | NSKNTLYLQMNSLRAEDTAVYYCTRGYDRKNYFEYWG | QSEDFAVYYCQQYNNWPPWTFGQGTKVEIK (SEQ ID |
| | QGTLVTVSS (SEQ ID NO: 141) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 13 | RQAPGKGLEWVSSIYSGPSYIYYADSVKGRFTISRDN | PGQAPRLLIYGASTRATGIPARFSGSGSGTEFTLTISSL |
| | PKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 142) | NO: 323) |
| PDAB | EVQLLESGGGLVQPGGSLRLSCAASGFTSSDYWMSWV | EIVMTQSPATLSVSPGERATLSCRASQSVSSNLAWYQQK |
| 14 | RQTPGKGLEWVSSIYSGPSYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 143) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 15 | RQTPGKGLEWVSSIYSGPSYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 144) | NO: 323) |
| PDAB | EVQLLESGGGLVQPGGSLRLSCVASGFTFSDYYMGWV | EIVMTQSPATLSVSPGERATLSCRASQSVSSNLAWYQQK |
| 16 | RQAPGKGLEWVSAIGTIVTYYADSVKGRFTISRDNSK | PGQAPRLLIYGASTRATGIPARFSGSGSGTEFTLTISSL |
| | NTLYLQMNSLRADDTAVYYCARGINYVDDWGQGTLVT | QSEDFAVYYCQQYNNWPPWTFGQGTKVEIK (SEQ ID |
| | VSS (SEQ ID NO: 145) | NO: 323) |
| PDAB | EVQLLESGGGLVQPGGSLRLSCAASGFTFSSYWMSWV | EIVMTQSPATLSVSPGERATLSCRASQSVSSNLAWYQQK |
| 17 | RQAPGKGLEWVSTIGSPGDTYYADSVKGRFTISRDNS | PGQAPRLLIYGASTRATGIPARFSGSGSGTEFTLTISSL |
| | KNTLYLQLNSLTAEDTAVYFCATGYAIFDYWGQGTLV | QSEDFAVYYCQQYNNWPPWTFGQGTKVEIK (SEQ ID |
| | TVSS (SEQ ID NO: 146) | NO: 323) |
| PDAB | EVQLLESGGGLVQPGGSLRLSCVASGFTFSDYYMSWV | EIVMTQSPATLSVSPGERATLSCRASQSVSSNLAWYQQK |
| 18 | RQAPGKGLEWVSAIGTIITYYADSVKGRFTISRDNSK | PGQAPRLLIYGASTRATGIPARFSGSGSGTEFTLTISSL |
| | NTLYLQMNSLRADDTAVYYCARGINFVDDWGQGTLVT | QSEDFAVYYCQQYNNWPPWTFGQGTKVEIK (SEQ ID |
| | VSS (SEQ ID NO: 147) | NO: 323) |
| PDAB | EVQLLESGGGLVQPGGSLRLSCAASGFTFSGYDMSWV | EIVMTQSPATLSVSPGERATLSCRASQSVSSNLAWYQQK |
| 19 | RQAPGKGLEWVSAIGWKSGSIYYADSVKGRFTISRDN | PGQAPRLLIYGASTRATGIPARFSGSGSGTEFTLTISSL |
| | SKNTLYLQMNSLRAEDTAVYYCAKGYNLKNYFDYWGQ | QSEDFAVYYCQQYNNWPPWTFGQGTKVEIK (SEQ ID |
| | GTLVTVSS (SEQ ID NO: 148) | NO: 323) |
| PDAB | EVQLLESGGDSVQPGESLRLSCAASGFTFSSSAMHWV | EIVMTQSPATLSVSPGERATLSCRASQSVSSNLAWYQQK |
| 20 | RQAPGKGLEWVSATNNDGSITYYADSVKGRFTISRDN | PGQAPRLLIYGASTRATGIPARFSGSGSGTEFTLTISSL |
| | SKNTLYLQMNGLRAEDTAVYYCARIIYYFDYWGQGTL | QSEDFAVYYCQQYNNWPPWTFGQGTKVEIK (SEQ ID |
| | VTVSS (SEQ ID NO: 149) | NO: 323) |
| PDAB | EVQLLESGGGLVQPGGSLRLSCAASGFIFSGYDMSWV | EIVMTQSPATLSVSPGERATLSCRASQSVSSNLAWYQQK |
| 21 | RQAPGKGLEWVSAISWTSGSIYYADSVKGRFTISRDN | PGQAPRLLIYGASTRATGIPARFSGSGSGTEFTLTISSL |
| | SKNTLYLQMNSLRAEDTAVYYCAKGYNRNNYEDYWGQ | QSEDFAVYYCQQYNNWPPWTFGQGTKVEIK (SEQ ID |
| | GTLVTVSS (SEQ ID NO: 150) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 22 | RQAPGKGLEWVSSIYSGGSSSRSYIYYADSVKGRFTI | PGQAPRLLIYGASTRATGIPARFSGSGSGTEFTLTISSL |
| | SRDNSKNTLYLQMSSLRAEDTAVYYCTRGWAYFDYWG | QSEDFAVYYCQQYNNWPPWTFGQGTKVEIK (SEQ ID |
| | QGTLVTVSS (SEQ ID NO: 151) | NO: 323) |
| PDAB | EVQLLESGGGLVQPGGSLRLSCAASGFTFSTYWMSWV | EIVMTQSPATLSVSPGERATLSCRASQSVSSNLAWYQQK |
| 23 | RQAPGKGLEWVSNIYSGGSTIDYADSVKGRFTISRDN | PGQAPRLLIYGASTRATGIPARFSGSGSGTEFTLTISSL |
| | SKNTLYLQMNSLRAEDTAVYYCARGWSYFDYWGQGTL | QSEDFAVYYCQQYNNWPPWTFGQGTKVEIK (SEQ ID |
| | VTVSS (SEQ ID NO: 152) | NO: 323) |
| PDAB | EVQLLESGGGLVQPGGSLRLSCAASGFIFSGYDMSWV | EIVMTQSPATLSVSPGERATLSCRASQSVSSNLAWYQQK |
| 24 | RQAPGKGPEWVSAITWDSGSTYYADSVKGRFTISRDN | PGQAPRLLIYGASTRATGIPARFSGSGSGTEFTLTISSL |
| | SKNTLYLQTNSLRAEDTAVYYCAKGYDRNNYFEYWGQ | QSEDFAVYYCQQYNNWPPWTFGQGTKVEIK (SEQ ID |
| | GTLVTVSS (SEQ ID NO: 153) | NO: 323) |
| PDAB | EVQLLESGGGLIQPGGSLRLSCAASGENESDYWMSWV | EIVMTQSPATLSVSPGERATLSCRASQSVSSNLAWYQQK |
| 25 | RQAPGKGLEWISAIYSGGYIYYADSVKGRFTISRDNS | PGQAPRLLIYGASTRATGIPARFSGSGSGTEFTLTISSL |
| | KNTLYLQMNSLRAEDTAVYYCTRGWSYCDYWGQGTLV | QSEDFAVYYCQQYNNWPPWTFGQGTKVEIK (SEQ ID |
| | TVSS (SEQ ID NO: 154) | NO: 323) |
| PDAB | EVQLLESGGGLVQPGGSLRLSCAASGENESDYWMSWV | EIVMTQSPATLSVSPGERATLSCRASQSVSSNLAWYQQK |
| 26 | RQAPGKGLEWVSAIYSGGYIYYADSVKGRFTISRDNS | PGQAPRLLIYGASTRATGIPARFSGSGSGTEFTLTISSL |
| | KNTLYLQMNSLRAEDTAVYYCARGWSYCDSWGQGTLV | QSEDFAVYYCQQYNNWPPWTFGQGTKVEIK (SEQ ID |
| | TVSS (SEQ ID NO: 155) | NO: 323 |
| PDAB | EVQLLESGGGLVQPGGSLRLSCAASGFTFSDHYMSWV | EIVMTQSPATLSVSPGERATLSCRASQSVSSNLAWYQQK |
| 27 | RQAPGKGLEWVSTISSGGNYIYYADSVKGRFTISRDS | PGQAPRLLIYGASTRATGIPARFSGSGSGTEFTLTISSL |
| | SKNTLYLQMNSLRAEDTAVYYCARILKNGKYIYYFDY | QSEDFAVYYCQQYNNWPPWTFGQGTKVEIK (SEQ ID |
| | WGQGTLVTVSS (SEQ ID NO: 156) | NO: 323) |
| PDAB | DVQLVESGGGVVRPGESLRLSCAASGFTFSSYDMSWV | QSVLTQPPSASGTPGQRVTISCSGSSSNIGSKYVSWYQQ |
| 28 | RQAPGEGLEWVSAISGSGVSAYYADSVKGRFTISRDN | LPGTAPKLLIYKDDQRPSGVPDRESGSKSGTSASLAISG |
| | SKNTLYLQMNSLRAEDTAIYYCAKDGLFFDYWGLGTL | LQSEDEADYYCAAWDGSLNGWVFGGGTKLTVL (SEQ |
| | VTVSS (SEQ ID NO: 157) | ID NO: 324) |
| PDAB | DVQLVESGGGVVRPGESLRLSCAASGFNFSSYDMNWV | QSVLTQPPSVSGTPGQRVTISCSGSNSNIGGYYVSWYQQ |
| 29 | RQAPGEGLQWVATITAAGDITYYADSVRGRFAISRDN | VPGTAPKLLIYKNFQRPSGVPDRFSGSKSGTSASLAISG |
| | SKNTLYLGLSSLRAEDTAIYYCGREPWNSFSDYWGLG | LQSEDEADYYCASWDGSLNGWVFGGGTKLTVL (SEQ |
| | TLVTVSS (SEQ ID NO: 158) | ID NO: 325) |
| PDAB | DVQLVESGGGVARPGEFLRLSCAASGFTFRDYDMGWV | QSVLTQPPSASGTPGQRVTISCSGSTSNIGTRYVYWYQQ |
| 30 | RQAPGEGLEWVSSISVSGTTYYADSVKGRFTISRDNS | LPGTAPKLLIYKNLQRPSGVPDRFSGSKSGTSASLAISG |
| | ENTLYLQMNSLTAEDTAVYYCGREDTLFHYWGLGTLV | LQSEDEADYYCAAWDERLNGWVFGGGTKLTVL (SEQ |
| | TVSS (SEQ ID NO: 159) | ID NO: 326) |
| PDAB | DVQLVESGGGVVRPGESLRLSCAASGFTFRNYDMTWV | QSVLTQPPSASGTPGQRVTISCSGSSSNIGRRYVYWYQQ |
| 31 | RQAPGEGLEWVATISGTDDTTYYADSVKGRATISRDN | LPGTAPKLLIYRNFERPSGVPDRESGSKSGTSASLAISG |
| | SNNTLYLRLFSLRAEDTAIYYCAREASNSFIDYWGLG | LQSEDEADYYCASWDESLNGWVFGGGTKLTVL (SEQ |
| | TLVTVSS (SEQ ID NO: 160) | ID NO: 327) |
| PDAB | DVQLVESGGGVVRPGESLRLSCAASGFTFSNYDMTWV | QSVLTQPPSASGTPGQRVTISCSGSTSNIGRRYVYWYQQ |
| 32 | RQAPGEGLEWVSSISPSAYSAYYADSVKGRFTISRDN | LPGTAPKLLIYRNFERPSGVPDRFSGSKSGTSASLAISG |
| | SKNTLYLEMNSLRAEDTAVYYCAKTSGNINYGLDYWG | LQSEDEADYYCASWDESLNGWVFGGGTKLTVL (SEQ |
| | LGTLVTVSS (SEQ ID NO: 161) | ID NO: 328) |
| PDAB | DVQLVESGGGVARPGESLRLSCAASGFTFRDYDMGWV | QSVLTQPPSASGTPGQRVTISCSGSTSNIGTRYVYWYQQ |
| 33 | RQAPGEGLEWVSSISVSGTTYYADSVKGRFTISRDNS | LPGTAPKLLIYKNLQRPSGVPDRESGSKSGTSASLAISG |
| | ENTLYLQMNSLTAEDTAVYYCGREDTLFHYWGLGTLV | LQSEDEADYYCAAWDERLNGWVFGGGTKLTVL (SEQ |
| | TVSS (SEQ ID NO: 162) | ID NO: 326) |
| PDAB | EVQLLESGGDLLQPGGSLRLSCSASGFDESSYYMTWV | EIVMTQSPATLSVSPGERATLSCRASQSVSSNLAWYQQK |
| 34 | RQAPGKGLEWVAAITSLGYTTYYANSVEGRETISRDN | PGQAPRLLIYGASTRATGIPARFSGSGSGTEFTLTISSL |
| | SENTLYLEMNSLRAEDTAVYYCATTHARGSRYFDYWG | QSEDFAVYYCQQYNNWPPWTFGQGTKVEIK (SEQ ID |
| | QGTLVTVSS (SEQ ID NO: 163) | NO: 323) |
| PDAB | EVQLLESGGGLLQPGGSLRLSCSASGFDESSYYMTWV | EIVMTQSPATLSVSPGERATLSCRASQSVSSNLAWYQQK |
| 35 | RQAPGKGLEWVAAITSLGYTTYYANSVEGRETISRDN | PGQAPRLLIYGASTRATGIPARFSGSGSGTEFTLTISSL |
| | SENTLYLEMNSLRAEDTAVYYCATTHARGSRYFDYWG | QSEDFAVYYCQQYNNWPPWTFGQGTKVEIK (SEQ ID |
| | QGTLVTVSS (SEQ ID NO: 164) | NO: 323) |
| PDAB | EVQLLESGGGLVQPGGSLRLSCAASGFSFSDYHMSWV | EIVMTQSPATLSVSPGERATLSCRASQSVSSNLAWYQQK |
| 36 | RQAPGKGLEWVSSISWNRGTTHYADSVRGRFTISRDN | PGQAPRLLIYGASTRATGIPARFSGSGSGTEFTLTISSL |
| | SKNTLYLQMNSLRAEDTAVYYCTRMQVYLMTSYFDYW | QSEDFAVYYCQQYNNWPPWTFGQGTKVEIK (SEQ ID |
| | GQGTLVTVSS (SEQ ID NO: 165) | NO: 323) |
| PDAB | EVQLLESGGGSVQPGGSLRLSCAASGFTFSDYYMSWV | EIVMTQSPATLSVSPGERATLSCRASQSVSSNLAWYQQK |
| 37 | RQAPGKGLEWVSAISWNNGRTYYADSVKGRFTISRDD | PGQAPRLLIYGASTRATGIPARFSGSGSGTEFTLTISSL |
| | SKNTLYLQMNSLTAEDTAVYYCAKMLVYLMTSYFDSW | QSEDFAVYYCQQYNNWPPWTFGQGTKVEIK (SEQ ID |
| | GQGTLVTVSS (SEQ ID NO: 166) | NO: 323) |
| PDAB | EVQLLESGGGLVQPGGSLRLSCAASGFSFSDYHMSWV | EIVMTQSPATLSVSPGERATLSCRASQSVSSNLAWYQQK |
| 38 | RQAPGKGLEWVSTISWNRGTTHYADSVKGRFTISRDN | PGQAPRLLIYGASTRATGIPARFSGSGSGTEFTLTISSL |
| | SKNTLYLQMNSLRAEDTAVYYCTRMQVYLMTSYFDYW | QSEDFAVYYCQQYNNWPPWTFGQGTKVEIK (SEQ ID |
| | GQGTLVTVSS (SEQ ID NO: 167) | NO: 323) |
| PDAB | EVQLLESGGGLVQPGGSLRLSCAASGFSFSDYHMSWV | EIVMTQSPATLSVSPGERATLSCRASQSVSSNLAWYQQK |
| 39 | RQAPGKGLEWVSTISWNRGTTHYADSVKGRLTISRDN | PGQAPRLLIYGASTRATGIPARFSGSGSGTEFTLTISSL |
| | SKNTLYLQMNSLRAEDTAVYYCTRMQVYLMTSYFDYW | QSEDFAVYYCQQYNNWPPWTFGQGTKVEIK (SEQ ID |
| | GQGTLVTVSS (SEQ ID NO: 168) | NO: 323) |
| PDAB | EVQLLESGGGSVQPGGSLRLSCAASGFTFSDYYMSWV | EIVMTQSPATLSVSPGERATLSCRASQSVSSNLAWYQQK |
| 40 | RQAPGKGLEWVSAISWNNGRMYYADSVKGRFTISRDD | PGQAPRLLIYGASTRATGIPARFSGSGSGTEFTLTISSL |
| | SKNTLYLQMNSLTAEDTAVYYCAKMLVYLMTSYFDSW | QSEDFAVYYCQQYNNWPPWTFGQGTKVEIK (SEQ ID |
| | GQGTLVTVSS (SEQ ID NO: 169) | NO: 323) |
| PDAB | EVQLLESGGGLVQPGGSLRLSCVGSGFVFDEYGIHWV | EIVMTQSPATLSVSPGERATLSCRASQSVSSNLAWYQQK |
| 41 | RQAPGKGLEWVAAIDWSGNRTYYADSVKGRFTISRDN | PGQAPRLLIYGASTRATGIPARFSGSGSGTEFTLTISSL |
| | SKNTAYLQMNSLTAEDTAVYYCAKFRWRDFYFEYWGQ | QSEDFAVYYCQQYNNWPPWTFGQGTKVEIK (SEQ ID |
| | GTLVTVSS (SEQ ID NO: 170) | NO: 323) |
| PDAB | EVQLLESGGGLVQPGGSLRLSCAASGFSFSDYHMSWV | EIVMTQSPATLSVSPGERATLSCRASQSVSSNLAWYQQK |
| 42 | RQAPGKGLEWVSSISWNRGTTHYADSVKGRFTISRDN | PGQAPRLLIYGASTRATGIPARFSGSGSGTEFTLTISSL |
| | SKNTLYLQMSSLRAEDTAVYYCTRMQVYLMTSYFDYW | QSEDFAVYYCQQYNNWPPWTFGQGTKVEIK (SEQ ID |
| | GQGTLVTVSS (SEQ ID NO: 171) | NO: 323) |
| PDAB | EVQLLESGGGLVQPGGSLRLSCVGSGFVFDEYGIHWV | EIVMTQSPATLSVSPGERATLSCRASQSVSSNLAWYQQK |
| 43 | RQAPGKGLEWVAAIDWSGNRTYYADSVKGRFTISRDN | PGQAPRLLIYGASTRATGIPARFSGSGSGTEFTLTISSL |
| | SKNTVYLQMNSLTAEDTAVYYCAKFRWRDFYFEYWGQ | QSEDFAVYYCQQYNNWPPWTFGQGTKVEIK (SEQ ID |
| | GTLVTVSS (SEQ ID NO: 172) | NO: 323) |
| PDAB | EVQLLESGGGLVQPGGSLRLSCVGSGFVEDEYGIHWV | EIVMTQSPATLSVSPGERATLSCRASQSVSSNLAWYQQK |
| 44 | RQAPGKGLEWVAAIDWSGNRTYYADSVKGRFTISRDN | PGQAPRLLIYGASTRATGIPARFSGSGSGTEFTLTISSL |
| | SRNTVYLQMNSLTAEDTAVYYCAKFRRREFYFEYWGQ | QSEDFAVYYCQQYNNWPPWTFGQGTKVEIK (SEQ ID |
| | GTLVTVSS (SEQ ID NO: 173) | NO: 323) |
| PDAB | EVQLLESGGGLVQPGGSLRLSCAASGFSFSDYHMSWV | EIVMTQSPATLSVSPGERATLSCRASQSVSSNLAWYQQK |
| 45 | RQAPGKGLEWVSSISWNRGTTHYADSVKGRFTISRDN | PGQAPRLLIYGASTRATGIPARFSGSGSGTEFTLTISSL |
| | SKNTLYLQMNSLRAEDTAVYYCTRMQVYLMTSYFDYW | QSEDFAVYYCQQYNNWPPWTFGQGTKVEIK (SEQ ID |
| | GQGTLVTVSS (SEQ ID NO: 174) | NO: 323) |
| PDAB | EVQLLESGGGLVQPGGSLRLSCAASGFTLSDYWMSWV | QSVLTQPPSASGTPGQRVTISCSGSSSNIRNNYVSWYQQ |
| 46 | RQAPGKGLEWVSDISWSAGDTYYYADSVKGRFTISRD | LPGTAPKLLIYKYNQRPSGVPDRESGSKSGTSASLAISG |
| | NSKNTLYLQMNSLRAEDTAVYYCAKYQRNGGYSFDYW | LQSEDEADYYCGTWDDSLNGFVFGGGTKLTVL (SEQ |
| | GQGTLVTVSS (SEQ ID NO: 175) | ID NO: 329) |
| PDAB | EVQLLESGGGLVQPGGSLRLSCAASGFTEDDYGMSWV | QSVLTQPPSASGTPGQRVTISCSGSSSNIGNNYVSWYQQ |
| 47 | RQAPGKGLEWVSAISWSGGRTYYADSVKGRFTISRDD | LPGTAPKLLIYKDDQRPSGVPDRFSGSKSGTSASLAISG |
| | SKNTLYLQMNSLRAEDTAVYYCTRDITILITYYFDYW | LQSEDEADYYCAARVDTLNVWVFGGGTKLTVL (SEQ |
| | GQGTLVTVSS (SEQ ID NO: 176) | ID NO: 330) |
| PDAB | EVQLLESGGGLVQPGGSLRLSCAASGEMENGSIMQWV | QSVLTQPPSASGTPGQRVTISCSGSSSNIRNNYVSWYQQ |
| 48 | RQAPGKGLEWVAGISDNGGTSYPDFVKGRFTISRDNS | LPGTAPKLLIYRNSQRPSGVPDRFSGSKSGTSASLAISG |
| | KNTVYLQMNSLRAEDTAVYYCVKDIDGYYFDYWGQGT | LQSEDEADYYCAAWDDSLNGWVFGGGTKLTVL (SEQ |
| | LVTVSS (SEQ ID NO: 177) | ID NO: 331) |
| PDAB | EVQLLESGGGLVQPGGSLRLSCAASGFTLSDYWMSWV | QSVLTQPPSASGTPGQRVTISCSGSSPNIRNNYVSWYQQ |
| 49 | RQAPGKGLEWVSDISWSAGDTYYYADSVKGRFTISRD | LPGTAPKLLIYKYNQRPSGVPDRFSGSKSGTSASLAISG |
| | NSKNTLYLQMNSLRAEDTAVYYCAKYQRNGGYSFDYW | LQSEDEADYYCGTWDDSLNGFVFGGGTKLTVL (SEQ |
| | GQGTLVTVSS (SEQ ID NO: 175) | ID NO: 332) |
| PDAB | EVQLLESGGGLVQPGGSLRLSCAVSGFTFSGSAMSWV | QSVLTQPPSASGTPGQRVTISCSGSTSNIGTNRVYWYQR |
| 50 | RQAPGKGREYVAGISSNGGTTYFTDSMKGRFTISRDN | LPGTAPKLLIYRDQERPSGVPDRFSGSKSGTSASLAISG |
| | SKNTLYLQMSSLRAEDTAVYYCARGGYNWNIYIDYWG | LQSEDEADYYCASWDDSLNAWVFGGGTKLTVL (SEQ |
| | QGTLVTVSS (SEQ ID NO: 178) | ID NO: 333) |
| PDAB | EVQLLESGGAFVQPGRSLGLSCAASGFTFSDYYMSWV | QSVLTQPPSASGAPGQRVTISCSGSYSNIGNSYVSWYQQ |
| 51 | RQAPGKGLEWVSVISNSGGSTYYTDSVKGRFTISRDN | PPGTAPKLLIYLNSQRPSGVPDRESGSKSGTSASLAISG |
| | SKNTLYLQMNSLRAEDTAVYYCTKDIGMTYFDYWGQG | LQSEDEADYYCAAWDDLNVWVFGGGTKLTVL (SEQ ID |
| | TLVTVSS (SEQ ID NO: 179) | NO: 334) |
| PDAB | EVQLLESGGALVQPGGSLRLSCAASGFTFSDYYMSWV | QSVLTQPPSASGTPGQRVTISCSGSYSNIGNSYVSWYQQ |
| 52 | RQAPGKGLEWVSVISNSGGSTYYADSVKGRFTISRDN | LPGTAPKLLIYLNSQRPSGVPDRESGSKSGTSASLAISG |
| | SKNTLYLQMNSLRAEDTAVYYCTKDIGMTYFDYWGQG | LRSEDEADYYCAAWDDLNVWVFGGGTKLTVL (SEQ ID |
| | TLVTVSS (SEQ ID NO: 180) | NO: 335) |
| PDAB | EVQLLESGGGLVQPGGSLRLSCAASGFDFSGSPMTWV | QSVLTQPPSASGTPGQRVTISCSGSRSNIGTKYVSWYQQ |
| 53 | RQAPGKGLEWVSVIRSKANSYATYYADSVKGRFTISR | LPGTAPKLLIYKDDQRPSGVPDRFSGSQSGTSASLAISG |
| | DNSKNTLYLQMNSLRAEDTAVYYCARGGTGYFDYWGQ | LQSEDEADYYCGTWDDSLNVWVFGGGTKLTVL (SEQ |
| | GTLVTVSS (SEQ ID NO: 181) | ID NO: 336) |
| PDAB | EVQLLESGGGLVQPGGSLRLSCAASGFTFSGSALSWV | QSVLTQPPSASGTPGQRVTISCSGSRSNIGTNRVYWYQQ |
| 54 | RQAPGKGLEWVSAIFSNGGSAYYADSVKGRFTISRDN | LPGTAPKLLIYKDDQRPSGVPDRFSGSKSGTSASLAISG |
| | SKNTLYLQMNSLRAEDTAVYYCAKGGYNWNNYLEYWG | LQSEDEADYYCAAWDDSLNGWVFGGGTKLTVL (SEQ |
| | QGTLVTVSS (SEQ ID NO: 182) | ID NO: 337) |
| PDAB | EVQLLESGGAFVQPGGSLRLSCAASGFTFSDYYMSWV | QSVLTQPPSASGTPGQRVTISCSGSYSNIGNSYVSWYQQ |
| 55 | RQAPGKGLEWVSVISNSGGSTYYTDSVKGRFTISRDN | LPGTAPKLLIYLNSQRPSGVPDRFSGSKSGTSASLAISG |
| | SKNTLYLQMNSLRAEDTAVYYCTKDIGMTYFDYWGQG | LQSEDEADYYCAAWDDPNVWVFGGGTKLTVL (SEQ ID |
| | TLVTVSS (SEQ ID NO: 183) | NO: 338) |
| PDAB | EVQLLESGGGLVQPGGSLRLSCAASGFTFSNYEMNWV | QSVLTQPPSASGTPGQRVTISCSGSSSNIDINYIDWYQQ |
| 56 | RQAPGKGLEWVGIIGTGGAITYYADSVKGRFTISRDN | LPGTAPKVLIYNTDQRPSGVPDRFSGSKSGTSASLAISG |
| | SKNTLYLQMNSLRAEDTAIYYCVKYSTESYFDYWGQG | LQSEDEADYYCAGWDSSLRVWVEGGGTKLTVL (SEQ |
| | TLVTVSS (SEQ ID NO: 184) | ID NO: 339) |
| PDAB | EVQLLESGGGLVQPGGSLRLSCAASGFDFSGYDMTWV | QSVLTQPPSASGTPGQRVTISCSGSTSNIGRNNYVSWYQ |
| 57 | RQAPGKGLEWVSSITWNSWIDGTKIYYADSVKGRFTI | QLPGTAPKLLIYRDYQRPSGVPDRFSGSKSGTSASLAIS |
| | SRDNSNNTLYLQMNSLRDEDTAIYYCAGGSLTVNYED | GLQSEDEADYYCVAWDDRVNGWVFGGGTKLTVL (SEQ |
| | YWGQGTLVTVSS (SEQ ID NO: 185) | ID NO: 340) |
| PDAB | EVQLLESGGGLVQPGGSLRLSCAASGFTFSDYWMTWV | QSVLTQPPSASGTPGQRVTISCSGSTSNIRNNYVSWYQQ |
| 58 | RQAPGKGLEWVSTISDSSNGGRTYYADSVKGRFTISR | LPGTAPKLLIYGKNQRPSGVPDRFSGSKSGTSASLAISG |
| | DNSKNTLYLQMNSLRAEDTAVYYCTKFDSWGQGALVT | LQSEDEADYYCATWDDSLNGWVFGGGTKLTVL (SEQ |
| | VSS (SEQ ID NO: 186) | ID NO: 341) |
| PDAB | EVQLLESGGGLVQPGGSLRLSCAASGFTFSDYWMTWV | QSVLTQPPSASGTPGQRVTISCSGSTSNIRNNYVSWYQQ |
| 59 | RQAPGKGLEWVSTISDSSNGGRTYYADSVKGRFTISR | LPGTAPKLLIYGKNQRPSGVPDRFSGSKSGTSASLAISG |
| | DNSKNTLYLQMNSLRAEDTAVYYCTKFDSWGQGALVT | LQSEDEADYYCSTWDDSLNGWVFGGGTKLTVL (SEQ |
| | VSS (SEQ ID NO: 186) | ID NO: 342) |
| PDAB | EVQLLESGGALVQPGGSLRLSCAASGFTFSDYYMSWV | QSVLTQPPSASGTPGQRVTISCSGSYSNIGNSYVSWYQQ |
| 60 | RQAPGKGLEWVSVISNSGGSTYYADSVKGRFTISRDN | LPGTAPKLLIYLNSQRPSGVPDRFSGSKSGTSASLAISG |
| | SKNTLYLQMNSLRAEDTAVYYCTKDIGMTYFDYWGQG | LQSEDEADYYCAAWDDLNVWVFGGGTKLTVL (SEQ ID |
| | TLVTVSS (SEQ ID NO: 180) | NO: 343) |
| PDAB | EVQLLESGGAFVQPGGSLRLSCAASGFTFSDYYMSWV | QSVLTQPPSASGAPGQRVTISCSGSYSNIGNSYVSWYQQ |
| 61 | RQAPGKGLEWVSVISNSGGSTYYTDSVKGRFTISRDN | LPGTAPKLLIYLNSQRPSGVPDRFSGSKSGTSASLAISG |
| | SKNTLYLQINSLRAEDTAVYYCTKDIGMTYFDYWGQG | LQSEDEADYYCAAWDDLNVWVFGGGTKLTVL (SEQ ID |
| | TLVTVSS (SEQ ID NO: 187) | NO: 344) |
| PDAB | EVQLLESGGAFVQPGGSLRLSCAASGFTFSDYYMSWV | QSVLTQPPSASGAPGQRVTISCSGSYSNIGNSYVSWYQQ |
| 62 | RQAPGKGLEWVSVISNSGGSTYYTDSVKGRFTISRDN | LPGTAPKLLIYLNSQRPSGVPDRFSGSKSGTSASLAISG |
| | SKNTLYLQMNSLRAEDTAVYYCTKDIGMTYFDYWGQG | LQSEDEADYYCAAWDDLNVWVFGGGTKLTVL (SEQ ID |
| | TLVTVSS (SEQ ID NO: 183) | NO: 344) |
| PDAB | EVQLLESGGGLVQPGGSLRLSCAASGFDFSGYDMTWV | QSVLTQPPSASGTPGQRVTISCSGSTSNIGRNNYVSWYQ |
| 63 | RQAPGKGLEWVSSITWNSWIDGTKIYYADSVKGRFTI | QLPGTAPKLLIYRDYQRPSGAPDRESGSKSGTSASLAIS |
| | SRDNSNNTLYLQMNSLRDEDTAIYYCAGGSLTVNYED | GLQSEDEADYYCVAWDDRVNGWVFGGGTKLTVL (SEQ |
| | YWGQGTLVTVSS (SEQ ID NO: 185) | ID NO: 345) |
| PDAB | EVQLLESGGGLVQPGGSLRLSCAASGFTFSGSALSWV | QSVLTQPPSASGTPGQRVTISCSGSRSNIGTNRVYWYQQ |
| 64 | RQAPGKGLEWVSAIFSNGGSAYYADSVKGRFTISRDN | LPGTAPKLLIYKDDQRPSGVPDRESGSKSGTSASLAISG |
| | SKNTLYLQMNSLRAEDTAVYYCAEGGYNWNNYLEYWG | LQSEDEADYYCASWDDSLNGWVFGGGTKLTVL (SEQ |
| | QGTLVTVSS (SEQ ID NO: 188) | ID NO: 346) |
| PDAB | EVQLLESGGGLVQPGGSLRLSCAASGFTFSDYDMAWV | QSVLTQPPSASGTPGQRVTISCSGSSFNIGTRYVYWYQQ |
| 65 | RQAPGKGLEWVSTITNTGSHIYYADSVKGRSTISRDN | LPGTAPKLLIYRNSQRPSGVPDRFSGSKSGTSASLAISG |
| | SENTLYLQMNSLRAEDTAVYYCVKEGTITIFDYWGQG | LQAEDEADYYCAAWDDSLNGWVFGGGTKLTVL (SEQ |
| | TLVTVSS (SEQ ID NO: 189) | ID NO: 347) |
| PDAB | EVQLLESGGGLVQPGGSLRLSCAASGFTFSGSALSWV | QSVLTQPPSASGTPGQRVTISCSGSRSNIGTNRVYWYQQ |
| 66 | RQAPGKGLEWVSAIFSNGGSAYYADSVKGRFTISRDN | LPGTAPKLLIYKDDQRPSGVPDRESGSKSGTSASLAISG |
| | SKNTLYLQMNSLRAEDTAVYYCAKGGYNWNNYLEYWG | LQSEDEADYYCASWDDSLNGWVFGGGTKLTVL (SEQ |
| | QGTLVTVSS (SEQ ID NO: 182) | ID NO: 346) |
| PDAB | EVQLLESGGGLVQPGGSLRLSCAASGFTFSDYWMSWV | EIVMTQSPATLSVSPGERATLSCRASQSVSSNLAWYQQK |
| 67 | RQAPGKGLEWVSSIYSGGSYIYYADSVKGRFTISRDN | PGQAPRLLIYGASTRATGIPARFSGSGSGTEFTLTISSL |
| | SKNTLYLQMSSLRAEDTAVYYCTRGWAYFDYWGQGTL | QSEDFAVYYCQQYNNWPPWTFGQGTKVEIK (SEQ ID |
| | VTVSS (SEQ ID NO: 190) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 68 | RQAPGKGLEWVSSIYSGPSYIYYADSVKGRFTISRDN | PGQAPRLLIYGASTRATGIPARFSGSGSGTEFTLTISSL |
| | SKNTLYLQMNSLGAEDTAVYYCTRGWTYSDYWGQGTL | QSEDFAVYYCQQYNNWPPWTFGQGTKVEIK (SEQ ID |
| | VTVSS (SEQ ID NO: 191) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 69 | RQAPGKGLEWVSSIYSGPSYIYYADSVKGRFTISRDN | PGQAPRLLIYGASTRATGIPARFSGSGSGTEFTLTISSL |
| | SKNTLYLQMNSLRAEDTAVYYCTRGWTYFDYWDQGTL | QSEDFAVYYCQQYNNWPPWTFGQGTKVEIK (SEQ ID |
| | VTVSS (SEQ ID NO: 192) | NO: 323) |
| PDAB | EVQLLESGGGLVQPGGSLRLSCAASGFIFSGYDMSWV | EIVMTQSPATLSVSPGERATLSCRASQSVSSNLAWYQQK |
| 70 | RQAPGKGLEWVSAIIWNSNTIYYADSVKGRFTISRDN | PGQAPRLLIYGASTRATGIPARFSGSGSGTEFTLTISSL |
| | SKNTVYLQMNSLRAEDTAVYYCARGYNLKNYFDYWGQ | QSEDFAVYYCQQYNNWPPWTFGQGTKVEIK (SEQ ID |
| | GTLVTVSS (SEQ ID NO: 193) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 71 | RQAPGKGLEWVSSIYSGGSYIYYADSVKGRFTISRDN | PGQAPRLLIYGASTRATGIPARFSGSGSGTEFTLTISSL |
| | SENTLYLQMSSLRAEDTAVYYCTRGWAYFDYWGQGTL | QSEDFAVYYCQQYNNWPPWTFGQGTKVEIK (SEQ ID |
| | VTVSS (SEQ ID NO: 194) | NO: 323) |
| PDAB | EVQLLESGGGLVQPGGSLRLSCAASGFIFSGYDMSWV | EIVMTQSPATLSVSPGERATLSCRASQSVSSNLAWYQQK |
| 72 | RQAPGKGLEWVSAIIWNSNTIYYADSVKGRFTISRDN | PGQAPRLLIYGASTRATGIPARFSGSGSGTEFTLTISSL |
| | SKNTVYLQMNSLRAEDTAVYYCVRGYNLKNYFDYWGQ | QSEDFAVYYCQQYNNWPPWTFGQGTKVEIK (SEQ ID |
| | GTLVTVSS (SEQ ID NO: 195) | NO: 323) |
| PDAB | EVQLLESGGGLVQPGGVLRLSCTASGFTEDDYGMHWV | EIVMTQSPATLSVSPGERATLSCRASQSVSSNLAWYQQK |
| 73 | RQAPEKGLEWVGAINWNGNVTHYADSVKGRFTISRDN | PGQAPRLLIYGASTRATGIPARFSGSGSGTEFTLTISSL |
| | SKNTLYLRMNSLRAEDTAVYYCAKNSGSEKRNYYFGY | QSEDFAVYYCQQYNNWPPWTFGQGTKVEIK (SEQ ID |
| | WGQGTLVTVSS (SEQ ID NO: 196) | NO: 323) |
| PDAB | EVQLLESGGGLVQPGGSLRLSCAASGFTFSDHYMSWV | EIVMTQSPATLSVSPGERATLSCRASQSVSSNLAWYQQK |
| 74 | RQAPGKGLEWVSTISSGGNYIYYADSVKGRFTISRDS | PGQAPRLLIYGASTRATGIPARFSGSGSGTEFTLTISSL |
| | SKNTLYLQMNSLRAEDTAVYYCARILKNGKYIYYFDY | QSEDFAVYYCQQYNNWPPWTFGQGTKVEIK (SEQ ID |
| | WGQGTLVTVSS (SEQ ID NO: 156) | NO: 323) |
| PDAB | EVQLLESGGGLVQPGGSLRLSCAASGFTFGDYWMSWV | EIVMTQSPATLSVSPGERATLSCRASQSVSSNLAWYQQK |
| 75 | RQAPGKGLEWVSTIYSGVATIYYADSVKGRFTISRDN | PGQAPRLLIYGASTRATGIPARFSGSGSGTEFTLTISSL |
| | SKNTLYLQMNSLRAEDTAVYYCARGWTYFDYWGQGTL | QSEDFAVYYCQQYNNWPPWTFGQGTKVEIK (SEQ ID |
| | VTVSS (SEQ ID NO: 197) | NO: 323) |
| PDAB | EVQLLESGGGLVQPGGSLRLSCTASGFTEDDYGMHWV | EIVMTQSPATLSVSPGERATLSCRASQSVSSNLAWYQQK |
| 76 | RQAPEKGLEWVGAINWNGNVTHYADSVKGRFTISRDN | PGQAPRLLIYGASTRATGIPARFSGSGSGTEFTLTISSL |
| | SKNTLYLRMNSLRAEDTAVYYCAKNSGSEKRNYYFGY | QSEDFAVYYCQQYNNWPPWTFGQGTKVEIK (SEQ ID |
| | WGQGTLVTVSS (SEQ ID NO: 198) | NO: 323) |
| PDAB | EVQLLESGGGLVQPGGPLRLSCAASGFTFSDYWMSWV | EIVMTQSPATLSVSPGERATLSCRASQSVSSNLAWYQQK |
| 77 | RQAPGKGLEWVSSIYTGGSSYIYYADSVKGRFTISRD | PGQAPRLLIYGASTRATGIPARFSGSGSGTEFTLTISSL |
| | NSKNTLYLQMNSLRAEDTAVYYCTRGWTYFDYWGQGT | QSEDFAVYYCQQYNNWPPWTFGQGTKVEIK (SEQ ID |
| | LVTVSS (SEQ ID NO: 199) | NO: 323) |
| PDAB | EVQLLESGGGLVQPGGSLRLSCTASGFTEDDYGMHWV | EIVMTQSPATLSVSPGERATLSCRASQSVSSNLAWYQQK |
| 78 | RQAPEKGLEWVGAVNWNGNVTHYADSVKGRFTISRDN | PGQAPRLLIYGASTRATGIPARFSGSGSGTEFTLTISSL |
| | SKNTLYLRMNSLRAEDTAVYYCAKNSGSEKRNYYFGY | QSEDFAVYYCQQYNNWPPWTFGQGTKVEIK (SEQ ID |
| | WGQGTLVTVSS (SEQ ID NO: 200) | NO: 323) |
| PDAB | EVQLLESGGGLVQPGGSLRLSCAASGFTEDDYWMSWV | EIVMTQSPATLSVSPGERATLSCRASQSVSSNLAWYQQK |
| 79 | RQAPGKGLEWVSAIYSGSYIYYADSVKGRFTISRDNS | PGQAPRLLIYGASTRATGIPARFSGSGSGTEFTLTISSL |
| | KNTLYLQMNSLRAEDTAVYYCARGWSYFDYWGQGTLV | QSEDFAVYYCQQYNNWPPWTFGQGTKVEIK (SEQ ID |
| | TVSS (SEQ ID NO: 201) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 80 | RQAPGKGLEWVSSIYSGTTYIYYADSVKGRFTISRDN | PGQAPRLLIYGASTRATGIPARFSGSGSGTEFTLTISSL |
| | SENTLYLQMNSLRAEDTAVYYCTRGWTYFDYRGQGTL | QSEDFAVYYCQQYNNWPPWTFGQGTKVEIK (SEQ ID |
| | VTVSS (SEQ ID NO: 202) | NO: 323) |
| PDAB | EVQLLESGGGLIQPGGSLRLSCAASGFTFSDYWMSWV | EIVMTQSPATLSVSPGERATLSCRASQSVSSNLAWYQQK |
| 81 | RQAPGKGLEWVSSIYSGSSYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTF GQGTKVEIK (SEQ ID |
| | VTVSS (SEQ ID NO: 203) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 82 | RQAPGKGLEWVSSIYSGVSYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 204) | NO: 323) |
| PDAB | EVQLLESGGGLVQPGGSLRLSCAASGFTFSGYWMSWV | EIVMTQSPATLSVSPGERATLSCRASQSVSSNLAWYQQK |
| 83 | RQAPGKGLEWVSSIYSGSSYIYYADSVKGRFTISRDK | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 205) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 84 | RQAPGKGLEWVSSIYSGPSYIYYADSVKGRFTISRDN | PGQAPRLLIYGASTRATGIPARFSGSGSGTEFTLTISSL |
| | SKNTLYLQMNSLRAEDTAVYYCTRGWTYFDYLGQGTL | QSEDFAVYYCQQYNNWPPWTFGQGTKVEIK (SEQ ID |
| | VTVSS (SEQ ID NO: 206) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 85 | RQAPGKGLEWVSSIYSGTTYIYYADSVKGRFTISRDN | PGQAPRLLIYGASTRATGIPARFSGSGSGTEFTLTISSL |
| | SENTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 130) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 86 | RQAPGKGLEWVSSIYTGGSSYIYYADSVKGRFTISRD | PGQAPRLLIYGASTRATGIPARFSGSGSGTEFTLTISSL |
| | NSKNTLYLQMNSLRAEDTAVYYCTRGWTYFDYWGQGT | QSEDFAVYYCQQYNNWPPWTFGQGTKVEIK (SEQ ID |
| | LVTVSS (SEQ ID NO: 207) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 87 | RQAPGKGLEWVSSIYSGATYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 208) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 88 | RQAPGKGLEWVSSIYTGGSYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 209) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 89 | RQAPGKGLEWVSSIYSGPSYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 135) | NO: 323) |
| PDAB | EVQLLESGGGLVQPGESLRLSCAASGFTFSDYWMSWV | EIVMTQSPATLSVSPGERATLSCRASQSVSSNLAWYQQK |
| 90 | RQAPGKGLEWVSSIYSGPSYIYYADSVKGRFTISRDN | PGQAPRLLIYGASTRATGIPARFSGSGSGTEFTLTISSL |
| | SKNTLYLQMNSLRAEDTAVYHCTRGWTYFDYWGQGTL | QSEDFAVYYCQQYNNWPPWTFGQGTKVEIK (SEQ ID |
| | VTVSS (SEQ ID NO: 210) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 91 | RQAPGKGLEWVSSIYSGPTYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 211) | NO: 323) |
| PDAB | EVQLLESGGGLVQPGGSLRLSCAASGFIFSGYDMSWV | EIVMTQSPATLSVSPGERATLSCRASQSVSSNLAWYQQK |
| 92 | RQAPGKGLEWVSAIIWNSNTIYYADSVKGRFTISRDN | PGQAPRLLIYGASTRATGIPARFSGSGSGTEFTLTISSL |
| | SKNTVYLRMNSLRAEDTAVYYCARGYNLKNYFDYWGQ | QSEDFAVYYCQQYNNWPPWTFGQGTKVEIK (SEQ ID |
| | GTLVTVSS (SEQ ID NO: 212) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 93 | RQAPGKGLEWVSSIYSGVSYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 204) | NO: 323) |
| PDAB | EVQLLESGGGLVQPGGSLRLSCAAPGFTFSDYWMSWV | EIVMTQSPATLSVSPGERATLSCRASQSVSSNLAWYQQK |
| 94 | RQAPGKGLEWVSSIYSGGSYIYYADSVKGRFTISRDN | PGQAPRLLIYGASTRATGIPARFSGSGSGTEFTLTISSL |
| | SKNTLYLQMSSLRAEDTAVYYCTRGWAYFDYWGQGTL | QSEDFAVYYCQQYNNWPPWTFGQGTKVEIK (SEQ ID |
| | VTVSS (SEQ ID NO: 213) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 95 | RQAPGKGLEWVSSIYSGGPYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 214) | NO: 323) |
| PDAB | EVQLLESGGGLVQPGGSLRLSCAASGFTEDDYWMSWV | EIVMTQSPATLSVSPGERATLSCRASQSVSSNLAWYQQK |
| 96 | RQAPGKGLEWVSTIYSGVATIYYADSVKGRFTISRDN | PGQAPRLLIYGASTRATGIPARFSGSGSGTEFTLTISSL |
| | SKNTLYLQMNSLRAEDTAVYYCARGWTYFDYWGQGTL | QSEDFAVYYCQQYNNWPPWTFGQGTKVEIK (SEQ ID |
| | VTVSS (SEQ ID NO: 215) | NO: 323) |
| PDAB | EVQLLEFGGGLVQPGGSLRLSCAASGFTEDDYWMSWV | EIVMTQSPATLSVSPGERATLSCRASQSVSSNLAWYQQK |
| 97 | RQAPGKGLEWVSTIYSGVATIYYADSVKGRFTISRDN | PGQAPRLLIYGASTRATGIPARFSGSGSGTEFTLTISSL |
| | SKNTLYLQMNSLRAEDTAVYYCARGWTYFDYWGQGTL | QSEDFAVYYCQQYNNWPPWTFGQGTKVEIK (SEQ ID |
| | VTVSS (SEQ ID NO: 216) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 98 | RQAPGKGLEWVSTIYSVGTIYYADSVKGRFTISRDNS | PGQAPRLLIYGASTRATGIPARFSGSGSGTEFTLTISSL |
| | KNTLYLQMDSLRAEDTAVYYCARGWTYFDYWGQGTLV | QSEDFAVYYCQQYNNWPPWTFGQGTKVEIK (SEQ ID |
| | TVSS (SEQ ID NO: 217) | NO: 323) |
| PDAB | EVQLLESGGGLVQPGGSLRLSCAASGFIFSGYDMSWV | EIVMTQSPATLSVSPGERATLSCRASQSVSSNLAWYQQK |
| 99 | RQAPGKGLEWVSAIGWKSGSIYYADSVKGRFTISRDN | PGQAPRLLIYGASTRATGIPARFSGSGSGTEFTLTISSL |
| | SKNTLYLQMNSLRAEDTAVYYCAKGYNLKNYFDYWGQ | QSEDFAVYYCQQYNNWPPWTFGQGTKVEIK (SEQ ID |
| | GTLVTVSS (SEQ ID NO: 218) | NO: 323) |
| PDAB | EVQLLESGGGLVQPGGSLRLPCAASGFTFSDYWMSWV | EIVMTQSPATLSVSPGERATLSCRASQSVSSNLAWYQQK |
| 100 | RQAPGKGLEWVSSIYSGVSYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 219) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 101 | RQAPGKGLEWVSSIYSGPSYIYYADSVKGRFTISRDN | PGQAPRLLIYGASTRATGIPARFSGSGSGTEFTLTISSL |
| | SKNTLYLQMNSPRAEDTAVYYCTRGWTYFDYWGQGTL | QSEDFAVYYCQQYNNWPPWTFGQGTKVEIK (SEQ ID |
| | VTVSS (SEQ ID NO: 220) | NO: 323) |
| PDAB | EVQLLESGGGLVQPGGSLRLSCAASGFIFSGYDMSWV | EIVMTQSPATLSVSPGERATLSCRASQSVSSNLAWYQQK |
| 102 | RQAPGKGLEWVSAISWTSDSIYYADSVKGRFTISRDN | PGQAPRLLIYGASTRATGIPARFSGSGSGTEFTLTISSL |
| | FENTLYLQMNSLRAEDTAVYYCAKGYNRNNYFDYWGQ | QSEDFAVYYCQQYNNWPPWTFGQGTKVEIK (SEQ ID |
| | GTLVTVSS (SEQ ID NO: 221) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 103 | RQAPGKGLEWVSFIYSGPSYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 222) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 104 | RQASGKGLEWVSSIYSGGSYIYYADSVKGRFTISRDN | PGQAPRLLIYGASTRATGIPARFSGSGSGTEFTLTISSL |
| | SKNTLYLQMSSLRAEDTAVYYCTRGWAYFDYWGQGTL | QSEDFAVYYCQQYNNWPPWTFGQGTKVEIK (SEQ ID |
| | VTVSS (SEQ ID NO: 223) | NO: 323) |
| PDAB | EVQLLESGGGLVQPGGSLRLSCAASGFTFSDYWMSWV | EIVMTQSPATLSVSPGERATLSCRASQSVSSNLAWYQQK |
| 105 | RQAPGKGLEWVSSIYSGASYIYYADSVKGRFTISRDN | PGQAPRLLIYGASTRATGIPARFSGSGSGTEFTLTISSL |
| | SKNTLYLQMNSLRAEDTAVYYCTRGWTYFDYWGQGTL | QSEDFAVYYCQQYNNWPPWTFGQGIKVEIK (SEQ ID |
| | VTVSS (SEQ ID NO: 224) | NO: 323) |
| PDAB | EVQLLESGGGLVQPGGSLRLSCAASGFTFSAYWMSWV | EIVMTQSPATLSVSPGERATLSCRASQSVSSNLAWYQQK |
| 106 | RQAPGKGLEWVSSIYSGTTYIYYADSVKGRFTISRDN | PGQAPRLLIYGASTRATGIPARFSGSGSGTEFTLTISSL |
| | SENTLYLQMNSLRAEDTAVYYCTRGWTYFDYWGQGTL | QSEDFAVYYCQQYNNWPPWTFGQGTKVEIK (SEQ ID |
| | VTVSS (SEQ ID NO: 225) | NO: 323) |
| PDAB | EVQLLESGGGLVQPGGSLRLSCAASGFTENDYWMSWV | EIVMTQSPATLSVSPGERATLSCRASQSVSSNLAWYQQK |
| 107 | RQAPGKELEWVSAIYSGGSSSYIYYADSVKGRFTISR | PGQAPRLLIYGASTRATGIPARFSGSGSGTEFTLTISSL |
| | DNSKNTLYLQMNSLRAEDTAVYYCARGWSYFDYWGQG | QSEDFAVYYCQQYNNWPPWTFGQGTKVEIK (SEQ ID |
| | TLVTVSS (SEQ ID NO: 226) | NO: 323) |
| PDAB | EVQLLESGGGSVQPGGSLRLSCAASGFAFSSYWMSWV | EIVMTQSPATLSVSPGERATLSCRASQSVSSNLAWYQQK |
| 108 | RQAPGKGLEWVSAMTGTSYIYYADSVKGRFTISRDNS | PGQAPRLLIYGASTRATGIPARFSGSGSGTEFTLTISSL |
| | KNTLYLQMNSLRAEDTAVYYCARGWAYLDYWGQGTLV | QSEDFAVYYCQQYNNWPPWTFGQGTKVEIK (SEQ ID |
| | TVSS (SEQ ID NO: 227) | NO: 323) |
| PDAB | EVQLLESGGGLVQPGGSLRLSCAASGFIFSGYDMSWV | EIVMTQSPATLSVSPGERATLSCRASQSVSSNLAWYQQK |
| 109 | RQAPGKGLEWVSAISWTSGSIYYADSVKGRFTISRDN | PGQAPRLLIYGASTRATGIPARFSGSGSGTEFTLTISSL |
| | SLNTLYLQMNSLRAEDTAVYYCAKGYNRNNYFDYWGQ | QSEDFAVYYCQQYNNWPPWTFGQGTKVEIK (SEQ ID |
| | GTLVTVSS (SEQ ID NO: 228) | NO: 323) |
| PDAB | DVQLVESGGGVVRPGESLRLSCAASGFNFSSYDMNWV | QSVLTQPPSASGTPGQRVTISCSGSTSNIGTRYVYWYQQ |
| 110 | RQAPGEGLQWVATITAAGDITYYADSVRGRFAISRDN | LPGTAPKLLIYRNFERPSGVPDRESGSKSGTSASLAISG |
| | SKNTLYLGLSSLRAEDTAIYYCGREPWNSFSDYWGLG | LQSEDEADYYCAAWDDRVNGWLFGGGTKLTVL (SEQ |
| | TLVTVSS (SEQ ID NO: 158) | ID NO: 348) |
| PDAB | DVQLVESGGGVVRPGESLRLSCAASGFTFSNFYMNWV | QSVLTQPPSASGTPGQRVTISCSGSSSNIGTNTVDWYQQ |
| 111 | RQAPGEGLEWVSTISGIDDTTWYADSVKGRFAISRDN | LPGTAPKLLIYDNFERPSGVPDRFSGSKSGISASLAISG |
| | SRNTLYLQMNSLRAEDTATYYCAKGTDELDYWGLGTL | LQSEDEADYYCGTWDDSLNAWVEGGGTKLTVL (SEQ |
| | VTVSS (SEQ ID NO: 229) | ID NO: 349) |
| PDAB | DVQLVESGGGVVRPGESLRLSCAASGFTFSDYDMAWV | QSVLTQPPSASGTPGQRVTISCSGSNSNIGRRYVYWYQQ |
| 112 | RQAPGEGLEWVATISGTDDSTYYADSVKGRATISRDN | LPGTAPKLLIYKNFERPSGVPDRFSGPKSGTSASLAISG |
| | SNNTLYLRLFSLRAEDTAIYYCAREASNSFIDYWGLG | LRSEDEADYYCASWDESLNGWVFGGGTKLTVL (SEQ |
| | TLVTVSS (SEQ ID NO: 230) | ID NO: 350) |
| PDAB | DVQLVESGGGVVRPGESLRLSCAASGFTFRNYDMTWV | QSVLTQPPSASGTPGQRVTISCSGSTSNIGRRYVYWYQQ |
| 113 | RQAPGEGLEWVATISGTDDTTYYADSVKGRATISRDN | LPGTAPKLLIYRNFERPSGVPDRFSGSKSGTSASLAISG |
| | SNNTLYLRLFSLRAEDTAIYYCAREASNSFIDYWGLG | LQSEDEADYYCGTWDDSLNSWVFGGGTKLTVL (SEQ |
| | TLVTVSS (SEQ ID NO: 160) | ID NO: 351) |
| PDAB | DVQLVESGGGVVRPGESLRLSCIASGFTFTTYDMSWV | QSVLTQPPSASGTPGQRVTISCSGSTFNIGTNYVSWYQQ |
| 114 | RQAPGEGLEWVSAIRPSGSVTYYADSVKGRFTISRDN | LPGTAPKLLIYKNHQRPSGVPDRESASKSGTSASLAISG |
| | SKNTLYLQMNSLRGEDTAIYYCATEASYSVEDWGLGT | LQSEDEADYYCAAWDDSLNVRVFGGGTKLTVL (SEQ |
| | LVTVSS (SEQ ID NO: 231) | ID NO: 352) |
| PDAB | DVQLVESGGGLVQPGGSLRLSCAASGFTENSYDMNWV | QSVLTQPPSASGTPGQRVTISCSGSSSNIGTRYVYWYQQ |
| 115 | RQAPGEGLEWVSTISGDGGDIYYADSVKGRFTISRDN | LPGTAPKLLIYRNFERPSGVPDRESGSKSGTSASLAISG |
| | SKNTLYLQMNSLRAEDTAIYYCGREGLNAFSDYWGLG | LQSEDEADYYCASWDDSLNGWVFGGGTKLTVL (SEQ |
| | TLVTVSS (SEQ ID NO: 232) | ID NO: 353) |
| PDAB | DVQLVESGGGVVRPGESLRLSCAASGFTFSNYDMTWV | QSVLTQPPSASGTPGQRVTISCSGSSSNIGRRYVYWYQQ |
| 116 | RQAPGEGLEWVATISGTDDSTYYADSVKGRATIFRDN | LPGTAPKLLIYRNFERPSGVPDRFSGSKSGTSASLAISG |
| | SNNTLYLRLLSLRAEDTAIYYCAREASNSFIDYWGLG | LQSEDEAYYYCASWDESLNGWVFGGGTKLTVL (SEQ |
| | TLVTVSS (SEQ ID NO: 233) | ID NO: 354) |
| PDAB | DVQLVESGGGVVRPGESLRLSCIASGFTFSTNDMSWV | QSVLTQPPSASGTPGQRVTISCSGTIFNIGTNYVSWYQQ |
| 117 | RQAPGEGLEWVSAIRPSGSVTYYADSVKGRFTISRDN | LPGTAPKLLIYLNSQRPSGVPDRESASKSGTSASLAISG |
| | SKNTLYLQMNSLRGEDTAIYYCAREASYSVEDWGLGT | LQSEDEADYYCAAWDDSLNVRVFGGGTKLTVL (SEQ |
| | LVTVSS (SEQ ID NO: 234) | ID NO: 355) |
| PDAB | DVQLVESGGGVVRPGESLRLSCAASGFTFSNYDMTWV | QSVLTQPPSASGTPGQRVTISCSGSTSNIGRRYVYWYQQ |
| 118 | RQAPGEGLEWVATISGTDYTTYYAASVKGRATISRDN | LPGTAPKLLIYRNFERPSGVPDRFSGSKSGTSASLAISG |
| | SNNTLYLRLFSLRAEDTAIYYCTREASNSFIDYWGLG | LQSEDEADYYCAAWDESLNGWVFGGGTKLTVL (SEQ |
| | TLVTVSS (SEQ ID NO: 235) | ID NO: 356) |
| PDAB | DVQLVESGGGVVRPGESLRLSCTASGFSFYNYAMNWV | QSVLTQPPSVSGTPGQRVTISCSGTISNIGNDNRVHWYQ |
| 119 | RQAPGEGLEFVADISGSGDTTSYAPAVKGRETISRDN | QLPGTAPKLLIYGNHQRPSGVPDRFSGSKSGTSASLAIS |
| | SKNTLYLQMNSLRAEDTAIYYCAKDSGDILFDYWGLG | GLQSEDEADYYCGTWDDSLNTWVFGGGTKLTVL (SEQ |
| | TLVTVSS (SEQ ID NO: 236) | ID NO: 357) |
| PDAB | DVQLVESGGGVVRPGESLRLSCAASGFTFSNYDMTWV | QSVLTQPPSASGTPGQRVTISCSGTTENIGRRYVYWYQQ |
| 120 | RQAPGEGLEWVATISGIDDSTYYADSVKGRATISRDN | LPGTAPKLLIYRNFERPSGVPDRFSGSKSGTSASLAISG |
| | SNNTLYLRLFSLRAEDTAIYYCAREASNSFIDYWGLG | LQSEDEADYYCASWDESLNGWVFGGGTKLTVL (SEQ |
| | TLVTVSS (SEQ ID NO: 237) | ID NO: 358) |
| PDAB | DVQLVESGGGVVRPGESLRLSCAASGFTFSSYAMNWV | QSVLTQPPSASGTPGQRVTISCSGSYSNIGNNYVYWYQQ |
| 121 | RQAPGEGLEYVADISGSGDATGYADSVKGRFTISRDN | LPGTAPKLLIYKDDQRPSGVPDRESGSKSGTSASLAISG |
| | SKNTLYLQMNSLRAEDTAIYYCAKDSGDIFFDYWGLG | LQSEDEADYYCGAFDDSLNVWVFGGGTKLTVL (SEQ |
| | TLVTVSS (SEQ ID NO: 238) | ID NO: 359) |
| PDAB | DVQLVESGGGVVRPGESLRLSCAASGFTFRNYDMAWV | QSVLTQPPSASGTPGQRVTISCSGSTSNIGRRYVYWYQQ |
| 122 | RQAPGEGLEWVATISGTDGTTYYADSVKGRATISRDN | LPGTAPKLLIYRNFERPSGVPDRFSGSKSGTSASLAISG |
| | SNNTLYLRLLSLRDEDTAIYYCAREASNSFIDYWGLG | LQSEDEADYYCASWDESLNGWVFGGGTKLTVL (SEQ |
| | TLVTVSS (SEQ ID NO: 239) | ID NO: 328) |
| PDAB | DVQLVESGGGVVRPGESLRLSCAASGFTFRNYDMTWV | QSVLTQPPSASGTPGQRVTISCSGSTSNIGRRYVYWYQQ |
| 123 | RQAPGEGLEWVATISGTDDTTYYADSVKGRATISRDN | LPGTAPKLLIYRNFERPSGVPDRESGSKSGTSASLAISG |
| | SNNTLYLRLFSLRAEDTAIYYCAREASNSFIDYWGPG | LQSEDEADYYCASWDESLNGWVFGGGTKLTVL (SEQ |
| | TLVTVSS (SEQ ID NO: 240) | ID NO: 328) |
| PDAB | DVQLVESGGGVVRPGESLRLSCAASGFTFSNYDMTWV | QSVLTQPPSASGTPGQRVTISCSGSSSNIGRRYVYWYQQ |
| 124 | RQAPGEGLEWVATISGTDDSTYYADSVKGRATIFRDN | LPGTAPKLLIYRNFERPSGVPDRFSGSKSGTSASLAISG |
| | SNNTLYLRLLSLRAEDTAIYYCAREASNSFIDYWGLG | LQSEDEADYYCASWDESLNGWVFGGGTKLTVL (SEQ |
| | TLVTVSS (SEQ ID NO: 233) | ID NO: 327) |
| PDAB | DVQLVESGGGVVRPGDSLTLSCAASGFTFSNYAMSWV | QSVLTQPPSASGTPGQRVTISCSGSTFNIQSNYVYWYQQ |
| 125 | RQAPGEGLEWVASISTNGGSTGYADSVKGRFTISRDN | LPGTAPKLLIYQSHVRPSGVPDRESGSKSGTSASLAISG |
| | SKNTLYLRLFSLRAEDTAIYYCTKETWNTSFDYWGLG | LQSEDEADYYCSSWDASLNGWVFGGGTKLTVL (SEQ |
| | TLVTVSS (SEQ ID NO: 241) | ID NO: 360) |
| PDAB | DVQLVESGGGVVRPGESLRLSCAASGFTFSNYDMTWV | QSVLTQPPSASGTPGQRVTISCSGSSSNIGRRYVYWYQQ |
| 126 | RQAPGEGLEWVTTISGTDDSTYYADSVKGRATIFRDN | LPGTAPKLLIYRNFERPSGVPDRFSGSKSGTSASLAISG |
| | SNNTLYLRLLSLRAEDTAIYYCAREASNSFIDYWGLG | LQSEDEADYYCASWDESLNGWVFGGGTKLTVL (SEQ |
| | TLVTVSS (SEQ ID NO: 242) | ID NO: 327) |
| PDAB | DVQLVESGGGVVRPGESLRLSCAASGFTFSNYDMTWV | QSVLTQPPSASGTPGQRVTISCSGSTSNIGRRYVYWYQQ |
| 127 | RQAPGEGLEWVATISGTDYTTYYAASVKGRATISRDN | LPGTAPKLLIYRNFERPSGVPDRESGSKSGTSASLAISG |
| | SNNTLYLRLFSLRAEDTAIYYCAREASNSFIDYWGLG | LQSEDEADYYCAAWDESLNGWVFGGGTKLTVL (SEQ |
| | TLVTVSS (SEQ ID NO: 243) | ID NO: 356) |
| PDAB | DVQLVESGGGVVRPGESLRLSCAASGENFSSYDMNWV | QSVLTQPPSASGTPGRRVTISCSGSTSNIGTRYVYWYQQ |
| 128 | RQAPGEGLQWVATITAAGDITYYADSVRGRFAISRDN | LPGTAPKLLIYRNFERPSGVPDRFSGSKSGTSASLAISG |
| | SKNTLYLGLSSLRAEDTAIYYCGREPWNSFSDYWGLG | LQSEDEADYYCAAWDDRVNGWLFGGGTKLTVL (SEQ |
| | TLVTVSS (SEQ ID NO: 158) | ID NO: 361) |
| PDAB | DVQLVESGGGVVRPGESLRLSCAASGFTFSNFYMNWV | QSVLTQPPSASGTPGQRVTISCSGGSSNIGTNTVDWYQQ |
| 129 | RQAPGEGLEWVSTISGIDDTTWYADSVKGRFTISRDN | LPGTAPKLLIYDNFERPSGVPDRESGSKSGTSASLAISG |
| | SRNTLYLQMNSLRAEDTATYYCAKGTDFLDYWGLGTL | LQSEDEADYYCGTWDDSLNAWVEGGGTKLTVL (SEQ |
| | VTVSS (SEQ ID NO: 244) | ID NO: 362) |
| PDAB | DVQLVESGGGVVRPGESLRLSCAASGFTFSDYDMAWV | QSVLTQPPSASGTPGQRVTISCSGSNSNIGRRYVYWYQQ |
| 130 | RQAPGEGLEWVATISGTDDSTYYADSVKGRATISRDN | LPGTAPKLLIYRNFERPSGVPDRFSGSKSGTSASLAISG |
| | SNNTLYLRLFSLRAEDTAIYYCAREASNSFIDYWGLG | LQSEDEADYYCASWDESLNGWVFGGGTKLTVL (SEQ |
| | TLVTVSS (SEQ ID NO: 230) | ID NO: 363) |
| PDAB | DVQLVESGGGVVRPGESLRLSCAASGFDFSNYDMNWV | QSVLTQPPSASGTPGQRVTMSCSGSSSNIGNRYVYWYQQ |
| 131 | RQAPGEGLEWVATISGSASITGTANITYYAPAVKGRF | FPGTAPKLLIHHNSQRPSGVPDRFSGSKSGTSASLAISG |
| | TISRDNSKNTLYLRLFSLRAEDTAIYYCARETFDGFF | LQSEDEADYYCASWDDSLNGWVFGGGTKLTVL (SEQ |
| | DYCGLGTLVTVSS (SEQ ID NO: 245) | ID NO: 364) |
| PDAB | DVQLVESGGGVVRPGESLRLSCAASGFTFRNYDMTWV | QSVLTQPPSASGTPGQRVTISCSGSTSNIGRRYVYWYQQ |
| 132 | RQAPGEGLEWVATISGTDDTTYYADSVKGRAAISRDN | LPGTAPKLLIYRNSQRPSGVPDRFSGSKSGTSASLAISG |
| | SNNTLYLRLLSLRAEDTAIYYCAREASNSFIDYWGLG | LQSEDEADYYCASWDESLNGWVFGGGTKLTVL (SEQ |
| | TLVTVSS (SEQ ID NO: 246) | ID NO: 365) |
| PDAB | DVQLVESGGGVVRPGESLRLSCAASGFTFSNFYMNWV | QSVLTQPPSASGTPGQRVTISCSGSSSNIGTNTVDWYQQ |
| 133 | RQAPGEGLEWVSTISGIDDTTWYADSVKGRFTISRDN | LPGTAPKLLIYDNFERPSGVPDRFSGSKSGTSASLAISG |
| | SRNTLYLQMNSLRAEDTATYYCAKGTDELDYWGLGTL | LQSEDEADYYCGTWDDSLNAWVFGGGTKLTVL (SEQ |
| | VTVSS (SEQ ID NO: 244) | ID NO: 366) |
| PDAB | DVQLVESGGGMVRPGESLRVSCAASGFDFSNYHMNWV | QSVLTQPPSASGTPGQRVTISCSGSNSNIGDHYVDWYQQ |
| 134 | RQAPGEGLEWVSRISDGDSRTYYSDFVKGRATISRDN | LPGTAPKLLIYNNVQRPSGVPDRFSGSKSGTSASLAISG |
| | SNNTLYLRLFSLRADDTAIYYCVRETLNNVEDYWGLG | LQSEDEADYYCATWDDNLNGWVFGGGTKLTVL (SEQ |
| | TLVTVSS (SEQ ID NO: 247) | ID NO: 367) |
| PDAB | DVQLVESGGGVVRPGESLRLSCAASGFTFSSHGMNWL | QSVLTQPPSASGTPGQRVTISCSGSSYNIDYNYIDWYQQ |
| 135 | RQAPGEGLESVAGIRSDGKYIYYADSVKGRATISRDD | LPGTAPKLLIYNSDQRPSGVPDRESGSKSGTSASLAISG |
| | SKSTVYLQMDSLRAEDTAIYYCARGWNFFDYWGPGAL | LQSEDEADYYCASWDAGLNVWMFGGGTKLTVL (SEQ |
| | VTVSS (SEQ ID NO: 248) | ID NO: 368) |
| PDAB | DVQLVDSGGGVVRPGESLRLSCAASGFTFRNYDMAWV | QSVLTQPPSASGTPGQRVTISCSGSTSNIGRRYVYWYQQ |
| 136 | RQAPGEGLEWVATISGTDGTTYYADSVKGRATISRDN | LPGTAPKLLIYRNFERPSGVPDRESGSKSGTSASLAISG |
| | SNNTLYLRLLSLRDEDTAIYYCAREASNSFIDYWGLG | LQSEDEADYYCASWDERLNGWVFGGGTKLTVL (SEQ |
| | TLVTVSS (SEQ ID NO: 249) | ID NO: 369) |
| PDAB | DVQSVESGGGVVRPGESLRLSCAASGFNFSSYDMNWV | QSVLTQPPSASGTPGQRVTISCSGSTSNIGTRYVYWYQQ |
| 137 | RQAPGEGLQWVATITAAGDITYYADSVRGRFTISRDN | LPGTAPKLLIYRNFERPSGVPDRESGSKSGTSASLAISG |
| | SKNTLYLGLSSLRAEDTAIYYCGREPWNSFSDYWGLG | LQSEDEADYYCAAWDDRVNGWLFGGGTKLTVL (SEQ |
| | TLVTVSS (SEQ ID NO: 250) | ID NO: 348) |
| PDAB | DVQLVESGGGVVRPGESLRLSCAASGENCSSYDMNWV | QSVLTQPPSASGTPGQRVTISCSGSTSNIGTRYVYWYQQ |
| 138 | RQAPGEGLQWVATITAAGDITYYADSVRGRFAISRDN | LPGTAPKLLIYRNFERPSGVPDRFSGSKSGTSASLAISG |
| | SKNTLYLGLSSLRAEDTAIYYCGREPWNSFSDYWGLG | LQSEDEADYYCAAWDDRVNGWLFGGGTKLTVL (SEQ |
| | TLVTVSS (SEQ ID NO: 251) | ID NO: 348) |
| PDAB | DVQLVESGGGVVRLGESLRLSCAASGENFIDYDMAWV | QSVLTQPPSASGTPGQRVTISCSGSTSNIGRRYVYWYQQ |
| 139 | RQAPGEGLEWVATISGTDDSTYYADSVKGRATISRDN | LPGTAPKLLIYRNFERPSGVPDRFSGSKSGTSASLAISG |
| | SNNTLYLRLFSLRAEDTAIYYCAREASNSFIGYWGLG | LQAEDEADYYCASWDESLNGWVFGGGTKLTVL (SEQ |
| | TLVTVSS (SEQ ID NO: 252) | ID NO: 370) |
| PDAB | DVQLVESGGGVVRPGESLRLSCAASGFTFSDYDMAWV | QSVLTQPPSASGTPGQRVTISCSGSNSSIGRRYVYWYQQ |
| 140 | RQAPGEGLEWVATISGTDDSTYYADSVKGRATISRDN | LPGTAPKLLIYRNFERPSGVPDRFSGSKSGTSASLAISG |
| | SNNTLYLRLFSLRAEDTAIYYCAREASNSFIDYWGLG | LQSEDEADYYCASWDESLNGWVFGGGTKLTVL (SEQ |
| | TLVTVSS (SEQ ID NO: 230) | ID NO: 371) |
| PDAB | DVQLVESGGGVVRPGESLRLSCAASGFTFRNYDMTWV | QSVLTQPPSASGTPGQRVTISCSGSTSNIGRRYVYWYQQ |
| 141 | RQAPGEGLEWVATISATDDTTYYADSVKGRATISRDN | VPGTAPKLLIYRNFERPSGVPDRFSGSKSGTSASLAISG |
| | SNNTLYLRLENLRAEDTAIYYCAREASNSFIDYWGLG | LQSEDEADYYCASWDESLNGWVFSGGTKLTVL (SEQ |
| | TLVTVSS (SEQ ID NO: 253) | ID NO: 372) |
| PDAB | DVQLVESGGGVVRPGESLRLSCAASGFNFSSYDMNWV | QSVLTQPPSASGTPGQRVTISCSGSTSNIGTRYVYWYQQ |
| 142 | RQAPGEGPQWVATITAAGDITYYADSVRGRFAISRDN | LPGTAPKLLIYRNFERPSGVPDRFSGSKSGTSASLAISG |
| | SKNTLYLGLSSLRAEDTAIYYCGREPWNSFSDYWGLG | LQSEDEADYYCAAWDDRVNGWLFGGGTKLTVL (SEQ |
| | TLVTVSS (SEQ ID NO: 254) | ID NO: 348) |
| PDAB | DVQLVESGGGVVRPGESLRLSCAASGFDFINYVMNWV | QSVLTQQPAPVSGTPGQRVTISCSGSSSNIGDHYVDWYQ |
| 143 | RQAPGEGLEFVARITNSGDRTWYADSVKGRFTISRDN | QLPGTAPKLLIYDNSQRPSGVPDRFSGSKSGTSASLAIS |
| | SNNTLYLRLFSLRAEDTAIYYCVRETSNYLLDYWGLG | GLQSEDEADYYCGTWDDDLNVWVFGGGTKLTVL (SEQ |
| | TLVTVSS (SEQ ID NO: 255) | ID NO: 373) |
| PDAB | DVQLVESGGGVVRPGESLGLSCAASGFTFRNYDMTWV | QSVLTQPPSASGTPGQRVTISCSGSTSNIGRRYVYWYQQ |
| 144 | RQAPGEGLGWVATISGTDDTTYYADSVKGRAAISRDN | FPGTAPKLLIYRNSQRPSGVPDRESGSKSGTSASLAISG |
| | SNNTLYLRLLSLRAEDTAIYYCAREASNSFIDYWGLG | LQSEDEADYYCASWDESLNGWVFGGGTKLTVL (SEQ |
| | TLVTVSS (SEQ ID NO: 256) | ID NO: 374) |
| PDAB | DVQLVESGGGVVRPGESLRLSCAASGFDFSNYDMNWV | QSVLTQPPSASGTPGQRVTMSCSGSSSNIGNRYVYWYRQ |
| 145 | RQAPGEGLEWVATISGSASITGTANITYYAPAVKGRF | FPGTAPKLLIHHNSQRPSGVPDRESGSKSGTSASLAISG |
| | TISRDNSKNTLYLRLFSLRAEDTAIYYCARETFDGFF | LQSEDEADYYCASWDDSLNGWVFGGGTKLTVL (SEQ |
| | DYCGLGTLVTVSS (SEQ ID NO: 245) | ID NO: 375) |
| PDAB | DVQLVESGGGVVRPGESLRLSCAASGFNFSSYDMNWV | QSVLTQPPSASGTPGQRVTISCSGSTSNIGTRYVYWYQQ |
| 146 | RQAPGEGLQWVATITAAGDITYYADSVRGRFAISRDN | LPGTAPKLLIYRNFERPSGVPDRFSGSKSGTSASLAISG |
| | SKNTLYLGLSSLRAKDTAIYYCGREPWNSFSDYWGLG | LQSEDEADYYCAAWDDRVNGWLFGGGTKLTVL (SEQ |
| | TLVTVSS (SEQ ID NO: 257) | ID NO: 348) |
| PDAB | DVQLVESGGGVVRPGDSLRLSCAASGFTFSDYDMTWV | QSVLTQPPSASGTPGQRVTISCSGSSSNIGTRYVYWYQQ |
| 147 | RQAPGEGLEWVSTISGSGDRIYYADSVKGRFTISRDN | VPGTAPKLLIYRNFERPSGVPDRFSGSKSGTSASLAISG |
| | SKNTLYLRLFSLRAEDTAIYYCAKEGWNSFIDYWGLG | LQSEDEADYYCAAWDDSLNGWVFGGGTKLTVL (SEQ |
| | TLVTVSS (SEQ ID NO: 258) | ID NO: 376) |
| PDAB | DVQLVESGGGVVRPGESLRLSCAASGENFSSYDMNWV | QSVLTQPPSASGTPGQRVTISCSGGTSNIGTRYVYWYQQ |
| 148 | RQAPGEGLQWVATITAAGDITYYADSVRGRFAISRDN | LPGTAPKLLIYRNFERPSGVPDRESGSKSGTSASLAISG |
| | SKNTLYLGLSSLRAEDTAIYYCGREPWNSFSDYWGLG | LQSEDEADYYCAAWDDRVNGWLFGGGTKLTVL (SEQ |
| | TLVTVSS (SEQ ID NO: 158) | ID NO: 377) |
| PDAB | DVQLVESGGGVVRPGESLRLSCAASGSNFSSYDMNWV | QSVLTQPPSASGTPGQRVTISCSGSTSNIGTRYVYWYQQ |
| 149 | RQAPGEGLQWVATITAAGDITYYADSVRGRFAISRDN | LPGTAPKLLIYRNFERPSGVPDRESGSKSGTSASLAISG |
| | SKNTLYLGLSSLRAEDTAIYYCGREPWNSFSDYWGLG | LQSEDEADYYCAAWDDRVNGWLFGGGTKLTVL (SEQ |
| | TLVTVSS (SEQ ID NO: 259) | ID NO: 348) |
| PDAB | DVQLVESGGGVVRPGESLRLSCAASGFTFSNYDMTWV | QSVLTQPPSASGTPGQRVTISCSGSTSNIGRRYVYWYQQ |
| 150 | RQAPGEGLEWVATISGTDYTTYYAASVKGRATISRDN | LPGTAPKLLIYRNFERPSGVPDRFSGSKSGTSASLAISG |
| | SNNTLYLRLFSLRAEDTAIYYCTREAPNSFIDYWGLG | LQSEDEADYYCAAWDESLNGWVFGGGTKLTVL (SEQ |
| | TLVTVSS (SEQ ID NO: 260) | ID NO: 356) |
| PDAB | DVQLVESGGGVVRPGESLRLSCAASGFDFINYVMNWV | QSVLTQQPASVSGTPGQRVTISCSGSSSNIGDHYVDWYQ |
| 151 | RQAPGEGLEFVARITNSGDRTWYADSVKGRFAISRDN | QLPGTAPKLLIYDNSQRPSGVPDRESGSKSGTSASLAIS |
| | SNNTLYLRLFSLRAEDTAIYYCVRETSNYLLDYWGLG | GLQSEDEADYYCGTWDDDLNVWVFGGGTKLIVL (SEQ |
| | TLVTVSS (SEQ ID NO: 261) | ID NO: 378) |
| PDAB | DVQLVESGGGVVRPGESLRLSCAASGENFIDYDMAWV | QSVLTQPPSASGTPGQRVTISCSGSTSNIGRRYVYWYQQ |
| 152 | RQAPGEGLEWVATISGTDDSTYYADSVKGRATISRDN | LPGTAPKLFIYRNFERPSGVPDRESGSKSGTSASLAISG |
| | SNNTLYLRLFSLRAEDTAIYYCAREASNSFIDYWGLG | LQAEDEADYYCASWDESLNGWVFGGGTKLTVL (SEQ |
| | TLVTVSS (SEQ ID NO: 262) | ID NO: 379) |
| PDAB | DVQLVESGGGVVRPGESLRLSCAASGENFSSYDMNWV | QSVLTQPPSASGTPGQRVTISCSGSTSNIGTRYVYWYQQ |
| 153 | RQAPGEGLQWVATITAAGDITYYAGSVRGRFAISRDN | LPGTAPKLLIYRNFERPSGVPDRFSGSKSGTSASLAISG |
| | SKNTLYLGLSSLRAEDTAIYYCGREPWNSFSDYWGLG | LQSEDEADYYCAAWDDRVNGWLFGGGTKLTVL (SEQ |
| | TLVTVSS (SEQ ID NO: 263) | ID NO: 348) |
| PDAB | DVQLVESGGGVVRPGESLRLSCVASGFTFGSDGMNWV | QSVLTQPPSASGTPGQRVTISCSGSNSNIDYNYIDWYQQ |
| 154 | RQAPGKGLDWISTISIDGTPTYYAESVKGRFTISRDN | LPGTAPKLLIYNNDQRP SEVPDRFSGSKSGTSASLAISG |
| | SKNTLYLQMNSLRAEDTAIYYCVKGYNFFDYWGLGTL | LQSEDEADYYCGTWDDSLNAWVEGGGTKLTVL (SEQ |
| | VTVSS (SEQ ID NO: 264) | ID NO: 380) |
| PDAB | DVQLVESGGGVVRPGESLRLSCAASGENFIDYDMAWV | QSVLTQPPSASGTPGQRVTISCSGSTSNIGRRYVYWYQQ |
| 155 | RQAPGEGLEWVATISGTDDSTYYADSVKGRATISRDN | LPGTAPKLLIYRNFERPSGVPDRFSGSKSGTSASLAISG |
| | SNNTLYLRLFSLRAEDTAIYYCAREASNSFIDYWGLG | LQAEDEADYYCASWDESLNGWVFGGGTKLTVL (SEQ |
| | TLVTVSS (SEQ ID NO: 262) | ID NO: 370) |
| PDAB | DVQLVESGGGVVRPGESLRLSCAASGENFIDYDMAWV | QSVLTQPPSASGTPGQRVTISCSGSTSNIGRRYVYWYQQ |
| 156 | RQAPGEGLEWVATISGTDDSTYYADSVKGRATISRDN | LPGTAPKLLIYRNFERPSGVPDRFSGSKSGTSASLAISG |
| | SNNTLYLRLFSLRAEDTAIYYCAREASNSFIDYWGLG | LQSEDEADYYCAAWDDSLNAWVFGGGTKLTVL (SEQ |
| | TLVTVSS (SEQ ID NO: 262) | ID NO: 381) |
| PDAB | DVQLVESGGGVVRPGESLRLSCAASGFTFSDYDMAWV | QSVLTQPPSASGTPGQRVTISCSGSNSNIGRRYVYWYQQ |
| 157 | RQAPGEGLEWVATISGTDDSTCYADSVKGRATISRDN | LPGTAPKLLIYRNFERPSGVPDRFSGSKSGTSASLAISG |
| | SNNTLYLRLFSLRAEDTAIYYCAREASNSFIDYWGLG | LQSEDEADYYCASWDESLNGWVFGGGTKLTVL (SEQ |
| | TLVTVSS (SEQ ID NO: 265) | ID NO: 363) |
| PDAB | DVQLVESGGGVVRPGESLRLSCAASGENFSSYDMNWV | QSVLTQPPSASGTPGQRVTISCSGSTSNIRTRYVYWYQQ |
| 158 | RQAPGEGLQWVATITAAGDITYYADSVRGRFAISRDN | LPGTAPKLLIYRNFERPSGVPDRESGSKSGTSASLAISG |
| | SKNTLYLGLSSLRAEDTAIYYCGREPWNSFSDYWGLG | LQSEDEADYYCAAWDDRVNGWLFGGGTKLTVL (SEQ |
| | TLVTVSS (SEQ ID NO: 158) | ID NO: 382) |
| PDAB | DVQLVESGGGVVRPGESLRLSCAASGENESSYDMNWV | QSVLTQPPSASGTPGQRVTISCSGSTSNIGTRYVYWYQQ |
| 159 | RQAPGEGLQWVATITAAGDITYYADSVRGRFAISRDN | LPGTAPKLLIYRNFERPSGVPDRFSGSKSGTSASLAISG |
| | SKNTLYLGLSSLRAEDTAIYYCGREPWNSFSDYWGLG | LQSEDEANYYCAAWDDSLNGWVFGGGTKLTVL (SEQ |
| | TLVTVSS (SEQ ID NO: 158) | ID NO: 383) |
| PDAB | DVQLVESGGGVVRPGESLRLSCVASGFTFGSDGMNWV | QSVLTQPPSASGTPGQRVTISCSGSNSNIDYNYIDWYQQ |
| 160 | RQAPGKGLDWISTISIDGTPTYYAESVKGRFTISRDN | LPGTAPKLLIYNNDQRPSEVPDRFSGSKSGTSASLAISG |
| | SKNTLYLQMNSLRAEDTAIYYCVKGYNFFDYWGLGTL | LQSEDEADYYCAAWDDNLNSWVEGGGTKLTVL (SEQ |
| | VTVSS (SEQ ID NO: 264) | ID NO: 384) |
| PDAB | DVQLVESGGGVVRPGESLRLSCVASGFTFGSDGMNWV | QSVLTQPPSASGTPGQRVTISCSGSNSNIDYNYIDWYQQ |
| 161 | RQAPGKGLDWISTISIDGTPTYYAESVKGRFTISRDN | LPGTAPKLLIYNNDQRP SEVPDRFSGSKSGTSASLAISG |
| | SKNTLYLQMNSLRAEDTAIYYCVKGYNFFDYWGLGTL | LRSEDEADYYCATWDDNLNSWVFGGGTKLIVL (SEQ |
| | VTVSS (SEQ ID NO: 264) | ID NO: 385) |
| PDAB | DVQLVESGGGVVRPGESLRLSCAASGFTFRNYDMTWV | QSVLTQPPSASGTPGQRVTISCSGSTSNIGRRYVYWYQQ |
| 162 | RQAPGEGLEWVATISATDDTTYYADSVKGRATISRDN | VPGTAPKLLIYRNFERPSGVPDRESGSKSGTSASLAISG |
| | SNNTLYLRLFNLRAEDTAIYYCAREASNSFIDYWGLG | LQSEDEADYYCASWDESLNGWVFGGGTKLTVL (SEQ |
| | TLVTVSS (SEQ ID NO: 253) | ID NO: 386) |
| PDAB | DVQLVESGGGVVRPGESLRLSCAASGFTFRNYDMTWV | QSVLTQPPSASGTPGQRVTISCSGSTSNIGRRYVYWYQQ |
| 163 | RQAPGEGLEWVATISGTDDTTYYADSVKGRAAISRDN | FPGTAPKLLIYRNSQRPSGVPDRFSGSKSGTSASLAISG |
| | SNNTLYLRLLSLRAEDTAIYYCAREASNSFIDYWGLG | LQSEDEADYYCASWDESLNGWVFGGGTKLTVL (SEQ |
| | TLVTVSS (SEQ ID NO: 246) | ID NO: 374) |
| PDAB | DVQLVESGGGVVRPGESLRLSCIASGFTFSSYDIEWV | QSVLTQPPSASGTPGQRVTISCSGSTENIGNNYVSWYQQ |
| 164 | RQAPGKGLEWVAAIDDDGSRRWYADSVKGRATISRDN | LPGTAPKVLIYKNLQRPSGVPDRESGSKSGTSASLAISG |
| | SESTVYLQLNSLRAEDTAVYYCTRATLYSSYDWGLGT | LQSEDEADYYCASWDDSLNVRVFGGGTKLTVL (SEQ |
| | LVTVSS (SEQ ID NO: 266) | ID NO: 387) |
| PDAB | DVQLVESGGGLVQPGGSLRLSCAASGFTFSDFAMGWV | EIVMTQSPATLSVSPGERATLSCRASQSVSSNLAWYQQK |
| 165 | RQAPGEGLEWVSTISGSGVITFYADSVKGRFTISRDN | PGQAPRLLIYGASTRATGIPARFSGSGSGTEFTLTISSL |
| | SKNTLYLQMNSLRVEDTAVYFCSEGMSYHDYWGLGTL | QSEDFAVYYCQQYNNWPPWTFGQGTKVEIK (SEQ ID |
| | VTVSS (SEQ ID NO: 267) | NO: 323) |
| PDAB | DVQLVESGGGVVQPGGSLRLSCAASGFTFSDFAMSWV | EIVMTQSPATLSVSPGERATLSCRASQSVSSNLAWYQQK |
| 166 | RQAPGEGLEWVSTISGSGVVTFYADSVKGRFTISRDN | PGQAPRLLIYGASTRATGIPARFSGSGSGTEFTLTISSL |
| | SKNALYLQMNSLRVEDTAVYFCSEGMSYHDYWGLGTL | QSEDFAVYYCQQYNNWPPWTFGQGTKVEIK (SEQ ID |
| | VTVSS (SEQ ID NO: 268) | NO: 323) |
| PDAB | DVQLVESGGGLVQPGGFLRLSCAASGFTFRDFAMSWV | EIVMTQSPATLSVSPGERATLSCRASQSVSSNLAWYQQK |
| 167 | RQAPGEGLEWVSSISHSGVITFYADSVKGRFTISRDN | PGQAPRLLIYGASTRATGIPARFSGSGSGTEFTLTISSL |
| | SKNTLYLQMNSLRVEDTAIYYCSEGLSYHDYWGLGTL | QSEDFAVYYCQQYNNWPPWTFGQGTKVEIK (SEQ ID |
| | VTVSS (SEQ ID NO: 269) | NO: 323) |
| PDAB | DVQLVESGGGVVQPGGSLRLSCAASGFTFRDFAMSWV | EIVMTQSPATLSVSPGERATLSCRASQSVSSNLAWYQQK |
| 168 | RQAPGEGLEWVSSISHSGVITFYADSVKGRFTISRDN | PGQAPRLLIYGASTRATGIPARFSGSGSGTEFTLTISSL |
| | SKNTLYLQMNSLRVEDTAVYFCSEGLSYHDYWGLGTL | QSEDFAVYYCQQYNNWPPWTFGQGTKVEIK (SEQ ID |
| | VTVSS (SEQ ID NO: 270) | NO: 323) |
| PDAB | DVQLVESGGGVVRPGESLRLSCAASGFTFSSDAMSWV | EIVMTQSPATLSVSPGERATLSCRASQSVSSNLAWYQQK |
| 169 | RQAPGEGLEWVSAISGSGVITYYADSVKGRFTISRDN | PGQAPRLLIYGASTRATGIPARFSGSGSGTEFTLTISSL |
| | SNNTLYLQMNSLRAEDTAIYYCATGFPFFDYWGLGTL | QSEDFAVYYCQQYNNWPPWTFGQGTKVEIK (SEQ ID |
| | VTVSS (SEQ ID NO: 271) | NO: 323) |
| PDAB | DVQLVESGGGVVRPGESLRLSCAASGFSFSSYDMSWV | EIVMTQSPATLSVSPGERATLSCRASQSVSSNLAWYQQK |
| 170 | RQAPGEGLEWVSSISGSGAITYYADSVKGRFTISRDN | PGQAPRLLIYGASTRATGIPARFSGSGSGTEFTLTISSL |
| | SKNTLYLQMNSLRAEDTAVYYCAESMIFFDYWGLGTL | QSEDFAVYYCQQYNNWPPWTFGQGTKVEIK (SEQ ID |
| | VTVSS (SEQ ID NO: 272) | NO: 323) |
| PDAB | DVQLVESGGGLVQPGGSLRLSCAASGFTFSDFAMSWV | EIVMTQSPATLSVSPGERATLSCRASQSVSSNLAWYQQK |
| 171 | RQAPGEGLEWVSTISGSGVIIFYADSVKGRFTISRDN | PGQAPRLLIYGASTRATGIPARFSGSGSGTEFTLTISSL |
| | SKNTLYLQMNSLRVEDTAVYFCSEGMSYHDYWGLGTL | QSEDFAVYYCQQYNNWPPWTFGQGTKVEIK (SEQ ID |
| | VTVSS (SEQ ID NO: 273) | NO: 323) |
| PDAB | DVQLVESGGGVVRPGESLRLSCAASGFSFDIYDMSWA | EIVMTQSPATLSVSPGERATLSCRASQSVSSNLAWYQQK |
| 172 | RQAPGKGLEWVSLISSSGVITYYADSVKGRFTISRDN | PGQAPRLLIYGASTRATGIPARFSGSGSGTEFTLTISSL |
| | SKSTLYLQMNSLRAEDTAVYYCARAGNTFFHYWGLGT | QSEDFAVYYCQQYNNWPPWTFGQGTKVEIK (SEQ ID |
| | LVTVSS (SEQ ID NO: 274) | NO: 323) |
| PDAB | DVQLVESGGGLVQPGGSLRLSCAASGFTFSSYAMSWV | EIVMTQSPATLSVSPGERATLSCRASQSVSSNLAWYQQK |
| 173 | RQAPGEGLEWVSSISHSGVITFYADSVKGRFTISRDN | PGQAPRLLIYGASTRATGIPARFSGSGSGTEFTLTISSL |
| | SKNTLYLQMNSLRVEDTAVYYCSEGLSYHDYWGLGTL | QSEDFAVYYCQQYNNWPPWTFGQGTKVEIK (SEQ ID |
| | VTVSS (SEQ ID NO: 275) | NO: 323) |
| PDAB | DVQLVESGGGVVRPGESLRLSCAASGFSFDIYDMSWV | EIVMTQSPATLSVSPGERATLSCRASQSVSSNLAWYQQK |
| 174 | RQAPGKGLEWVSLISSSGVITYYADSVKGRFTISRDN | PGQAPRLLIYGASTRATGIPARFSGSGSGTEFTLTISSL |
| | SKSTLYLQMNSLRAEDTAIYYCARAGNTFFHYWGLGT | QSEDFAVYYCQQYNNWPPWTFGQGTKVEIK (SEQ ID |
| | LVTVSS (SEQ ID NO: 276) | NO: 323) |
| PDAB | DVQLVESGGGLVQPGGSLRLSCAASGFTFSDFAMSWV | EIVMTQSPATLSVSPGERATLSCRASQSVSSNLAWYQQK |
| 175 | RQAPGEGLEWVSSISHSGVITFYADSVKGRFTISRDN | PGQAPRLLIYGASTRATGIPARFSGSGSGTEFTLTISSL |
| | SKNTLYLQMNSLRVEDTAIYFCSEGLSYHDYWGLGTL | QSEDFAVYYCQQYNNWPPWTFGQGTKVEIK (SEQ ID |
| | VTVSS (SEQ ID NO: 277) | NO: 323) |
| PDAB | DVQLVESGGGVVRPGESLRLSCVASGFTFSTDTMSWV | EIVMTQSPATLSVSPGERATLSCRASQSVSSNLAWYQQK |
| 176 | RQAPGEGLEWVSASSGSGVITYYADSAKGRFTISRDN | PGQAPRLLIYGASTRATGIPARFSGSGSGTEFTLTISSL |
| | SNNTLYLQMNSLRAEDTAIYYCATGFPFFDYWGLGTL | QSEDFAVYYCQQYNNWPPWTFGQGTKVEIK (SEQ ID |
| | VTVSS (SEQ ID NO: 278) | NO: 323) |
| PDAB | DVQLVESGGGLVQPGGSLRLSCAASGFTFSDFAMSWI | EIVMTQSPATLSVSPGERATLSCRASQSVSSNLAWYQQK |
| 177 | RQAPGEGLEWVSTISGSGVITFYADSVKGRFTISRDN | PGQAPRLLIYGASTRATGIPARFSGSGSGTEFTLTISSL |
| | SKNTLYLQMNSLRVEDTAVYFCSEGMSYHDYWGLGTL | QSEDFAVYYCQQYNNWPPWTFGQGTKVEIK (SEQ ID |
| | VTVSS (SEQ ID NO: 279) | NO: 323) |
| PDAB | DVQLVESGGGLVQPGGSLRLSCAASGFTFSDFAVSWV | EIVMTQSPATLSVSPGERATLSCRASQSVSSNLAWYQQK |
| 178 | RQAPGEGLEWVSTISGSGVITFYADSVKGRFTISRDN | PGQAPRLLIYGASTRATGIPARFSGSGSGTEFTLTISSL |
| | SKNTLYLQMNSLRVEDTAVYFCSEGMSYHDYWGLGTL | QSEDFAVYYCQQYNNWPPWTFGQGTKVEIK (SEQ ID |
| | VTVSS (SEQ ID NO: 280) | NO: 323) |
| PDAB | DVQLVESGGGVVRPGESPRLSCVASGFTFSTDTMSWV | EIVMTQSPATLSVSPGERATLSCRASQSVSSNLAWYQQK |
| 179 | RQAPGEGLEWVSASSGSGVITYYADSVKGRFTISRDN | PGQAPRLLIYGASTRATGIPARFSGSGSGTEFTLTISSL |
| | SNNTLYLQMNSLRAEDTAIYYCATGFPFFDYWGLGTL | QSEDFAVYYCQQYNNWPPWTFGQGTKVEIK (SEQ ID |
| | VTVSS (SEQ ID NO: 281) | NO: 323) |
| PDAB | DVQLVESGGGVVRPGESLRLSCAASGFTFSSDAVSWV | EIVMTQSPATLSVSPGERATLSCRASQSVSSNLAWYQQK |
| 180 | RQAPGEGLEWVSSISGSGVITYYADSVKGRFTISRDN | PGQAPRLLIYGASTRATGIPARFSGSGSGTEFTLTISSL |
| | SKNTLYLQMNSLRAEDTAVYYCAKDVFFFNYWGLGTL | QSEDFAVYYCQQYNNWPPWTFGQGTKVEIK (SEQ ID |
| | VTVSS (SEQ ID NO: 282) | NO: 323) |
| PDAB | DVQLVESGGGLVQPGGSLRLSCAASGFTFSDFAMSWV | EIVMTQSPATLSVSPGERATLSCRASQSVSSNLAWYQQK |
| 181 | RQAPGEGLEWVSSISHSGVITFYADSVKGRFTISRDN | PGQAPRLLIYGASTRATGIPARFSGSGSGTEFTLTISSL |
| | SKNTLYLQMNSLRVEDTAIYYCSEGLSYHDYWGLGTL | QSEDFAVYYCQQYNNWPPWTFGQGTKVEIK (SEQ ID |
| | VTVSS (SEQ ID NO: 283) | NO: 323) |
| PDAB | DVQLVESGGGVVRPGESLRLSCAASGFTFSSDAMSWV | EIVMTQSPATLSVSPGERATLSCRASQSVSSNLAWYQQK |
| 182 | RQAPGEGLEWVSSISGSGVITYYADSVKGRFTISRDN | PGQAPRLLIYGASTRATGIPARFSGSGSGTEFTLTISSL |
| | SKNTLYLQMNSLRAEDTAVYYCAKDVFFFNYWGLGTL | QSEDFAVYYCQQYNNWPPWTFGQGTKVEIK (SEQ ID |
| | VTVSS (SEQ ID NO: 284) | NO: 323) |
| PDAB | DVQLVESGGGVVRPGESLRLSCVASGFTFSSDVMSWV | EIVMTQSPATLSVSPGERATLSCRASQSVSSNLAWYQQK |
| 183 | RQAPGEGLEWVSASSGSGVITYYADSVKGRFTISRDN | PGQAPRLLIYGASTRATGIPARFSGSGSGTEFTLTISSL |
| | SKNTLYLQMNSLRAEDTAVYYCAKAGNTFLDYWGLGT | QSEDFAVYYCQQYNNWPPWTFGQGTKVEIK (SEQ ID |
| | LVTVSS (SEQ ID NO: 285) | NO: 323) |
| PDAB | DVQLVESGGGVVRPGESLRLSCAASGFSFDIYDMSWV | EIVMTQSPATLSVSPGERATLSCRASQSVSSNLAWYQQK |
| 184 | RQAPGKGLEWVSLISSSGVITYYADSVKGRFTISRDN | PGQAPRLLIYGASTRATGIPARFSGSGSGTEFTLTISSL |
| | SKSTLYLQMNSLRAEDTAVYYCARAGNTFFHYWGLGT | QSEDFAVYYCQQYNNWPPWTFGQGTKVEIK (SEQ ID |
| | LVTVSS (SEQ ID NO: 286) | NO: 323) |
| PDAB | DVQLVESGGGLVQPGGSLRLSCAASGFTFSDFAMSWV | EIVMTQSPATLSVSPGERATLSCRASQSVSSNLAWYQQK |
| 185 | RQAPGEGLEWVSTISGSGVITFYADSVKGRFTISRDN | PGQAPRLLIYGASTRATGIPARFSGSGSGTEFTLTISSL |
| | SKNALYLQMNSLRVEDTAVYFCSEGMSYHDYWGLGTL | QSEDFAVYYCQQYNNWPPWTFGQGTKVEIK (SEQ ID |
| | VTVSS (SEQ ID NO: 287) | NO: 323) |
| PDAB | DVQLVESGGGVVRPGESLRLSCVASGFTFSTDTMSWV | EIVMTQSPATLSVSPGERATLSCRASQSVSSNLAWYQQK |
| 186 | RQAPGEGLEWVSASSGSGVITYYADSVKGRFTISRDN | PGQAPRLLIYGASTRATGIPARFSGSGSGTEFTLTISSL |
| | SNNTLYLQMNSLRAEDTAIYYCATGFPFFDYWGLGTL | QSEDFAVYYCQQYNNWPPWTFGQGTKVEIK (SEQ ID |
| | VTVSS (SEQ ID NO: 288) | NO: 323) |
| PDAB | DVQLVESGGGLVQPGGSLRLSCAASGFTFSDFAMSWV | EIVMTQSPATLSVSPGERATLSCRASQSVSSNLAWYQQK |
| 187 | RQAPGEGLEWVSTISGSGVITFYADSVKGRFTISRDN | PGQAPRLLIYGASTRATGIPARFSGSGSGTEFTLTISSL |
| | SKNTLYLQMNSLRVEDTAVYFCSEGMSYHDYWGLGTL | QSEDFAVYYCQQYNNWPPWTFGQGTKVEIK (SEQ ID |
| | VTVSS (SEQ ID NO: 289) | NO: 323) |
| PDAB | QVTLKESGPGILQPSQTLSLTCSFSGFSLSTFGMGVG | QAVVTQESALTTSPGETVILTCRSSTGAVTTSNYANWVQ |
| 188 | WIRQPSGKGLEWLAHIWWDDDKYYNPALKSRLTISKD | EKPDHLFTGLIGGINNRAPGVPARFSGSLIGDKAALTIT |
| | TSKNQVFLKIANVDTADTATYYCARIITTAWYFDVWG | GAQTEDEAIYFCALWYSNHFIFGSGTKVTVL (SEQ ID |
| | TGTTVTVSS (SEQ ID NO: 290) | NO: 388) |
| PDAB | QIQLVQSGPELKKPGETVKISCKASGYTFTTYGMSWV | DIQMTQSPASLSASVGETVTITCRASENIYSYLAWYQQK |
| 189 | KQAPGKGLKWMGWINTYSGVPTYADDFKGRFAFSLET | QGKSPQLLVYNAKTLAEGVPSRFSGSGSGTQFSLKINSL |
| | SASTAYLQINNLKNEDTATYFCARPRRQVFDYWGQGT | QPEDFGSYYCQHHYGTPFTFGSGTKLEIK (SEQ ID |
| | TLTVSS (SEQ ID NO: 291) | NO: 389) |
| PDAB | QVKLQQSGAELVRPGASVKLSCKASGYTFTDYYINWI | DIVLTQSPASLAVSLGQRATISCRASKSVSTSGYSYMHW |
| 190 | KQRPGQGLEWIARIYPVSGSTYYNDKFKGKATLTAEK | YQQKPGQPPKLLIYLASNLESGVPARFSGSGSGTDFTLN |
| | SSSTAYMQLSSLISEDSAVYFCVHIYYGNHPYYFDFW | IHPVEEEDAATYYCQHSRELYTFGGGTKLEIK (SEQ |
| | GQGTTLTVSS (SEQ ID NO: 292) | ID NO: 390) |
| PDAB | QVQLQQPGAELVKPGTSVKMSCKASGYTFITYWITWV | QAVVTQESALTTSPGETVTLTCRSSTGAVTTSNYANWVQ |
| 191 | KQRPGHGLEWIGDIYPGSGSTNYNAKFRSKATLTVDT | EKPDHLFTGLIGGTNNRAPGVPARFSGSLIGDKAALTIT |
| | SSSTAYMQFISLTSEDSAVYYCALKEIVPDYWGQGTT | GAQTEDEAIYFCALWYSNHLVFGGGTKLTVL (SEQ ID |
| | LTVSS (SEQ ID NO: 293) | NO: 391) |
| PDAB | QVQLQQPGAELVKPGTSVKMSCKASGYTFITYWITWV | DIVLTQSPASLAVSLGQRATISCRASQSVSTSSYSYMHW |
| 192 | KQRPGHGLEWIGDIYPGSGSTNYNAKFRSKATLTVDT | YQQKPGQPPKLLIKYASNLESGVPARFSGSGSGTDFTLN |
| | SSSTAYMQFISLISEDSAVYYCALKEIVPDYWGQGTT | IHPVEEEDTATYYCQHSWEIPFTFGSGTKLEIK (SEQ |
| | LTVSS (SEQ ID NO: 293) | ID NO: 392) |
| PDAB | QVQLQQPGAELVKPGASVKLSCKASGYTFTSYWMHWV | QAVVTQESALTTSPGETVTLTCRSSTGAVTTSNYANWVQ |
| 193 | KQRPGQGLEWIGMIHPNSGSTNYNEKFKSKATLTVDK | EKPDHLFTGLIGGINNRAPGVPARFSGSLIGDKAALTIT |
| | SSSTAYMQLSSLISEDSAVYYCARSRGNYVWYFDVWG | GAQTEDEAIYFCALWYSNHSWVFGGGTKLTVL (SEQ |
| | TGTTVTVSS (SEQ ID NO: 294) | ID NO: 393) |
| PDAB | QVTLKESGPGILQPSQTLSLTCSFSGFSLSTFGMGVG | ENVLTQSPAIMSASLGEKVTMSCRASSSVNYMYWYQQKS |
| 194 | WIRQPSGKGLEWLAHIWWDDDKYYNPALKSRLTISKD | DASPKLWIYYTSNLAPGVPARFSGSGSGNSYSLTISSME |
| | TSKNQVFLKIANVDTADTATYYCARMNPRNYFDYWGQ | GEDAATYYCQQFTSSPSTEGGGTKLEIK (SEQ ID |
| | GTTLTVSS (SEQ ID NO: 295) | NO: 394) |
| PDAB | QVTLKESGPGILQPSQTLSLTCSFSGFSLSTFGMGVG | QAVVTQESALTTSPGETVTLTCRSSTGAVTTSNYANWVQ |
| 195 | WIRQPSGKGLEWLAHIWWDDDKYYNPALKSRLTISKD | EKPDHLFTGLIGGTNNRAPGVPARFSGSLIGDKAALTIT |
| | TSKNQVFLKIANVDTADTATYYCARMNPRNYFDYWGQ | GAQTEDEAIYFCALWYSNRWVEGGGTKLIVL (SEQ ID |
| | GTTLTVSS (SEQ ID NO: 295) | NO: 395) |
| PDAB | QVQLQQPGAELVKPGASVKVSCKASGYTFTSYWMHWV | ENVLTQSPAIMSASLGEKVTMSCRASSSVNYMYWYQQKS |
| 196 | KQRPGQGLEWIGRIHPSDSDTNYNQKFKGKATLTVDK | DASPKLWIYYTSNLAPGVPARFSGSGSGNSYSLTISSME |
| | SSSTAYMQLSSLTSEDSAVYYCAIRDWDLDYWGQGTT | GEDAATYYCQQFTSSPSTFGGGTKLEIK (SEQ ID |
| | LTVSS (SEQ ID NO: 296) | NO: 394) |
| PDAB | QVQLQQPGAELVKPGASVKVSCKASGYTFTSYWMHWV | QAVVTQESALTTSPGETVTLTCRSSTGAVTTSNYANWVQ |
| 197 | KQRPGQGLEWIGRIHPSDSDTNYNQKFKGKATLTVDK | EKPDHLFTGLIGGTNNRAPGVPARFSGSLIGDKAALTIT |
| | SSSTAYMQLSSLISEDSAVYYCAIRDWDLDYWGQGTT | GAQTEDEAIYFCALWYSNRWVFGGGTKLTVL (SEQ ID |
| | LTVSS (SEQ ID NO: 296) | NO: 395) |
| PDAB | EVMLVESGGGLVKPGGSLKLSCAASGFTFSSYLMSWV | DIQMTQSPASQSASLGESVTITCLASQTIGTWLAWYQQK |
| 198 | RQTPEKRLEWVASISGGGRDTYYPDSVKGRFTISRDN | PGKSPQVLIYAATSLADGVPSRFSGSGSGTKFSFKISSL |
| | AKNTLYLQMSSLRSEDTALYYCARHGVGAMNYWGQGT | QAEDFVSYYCQQLYSTPWTFGGGTKLEIK (SEQ ID |
| | SVTVSS (SEQ ID NO: 297) | NO: 396) |
| PDAB | QVTLKESGPGILQPSQTLSLTCSFSGFSLSTFGMGVG | QAVVTQESALTTSPGETVTLTCRSSTGAVTTSNYANWVQ |
| 199 | WIRQPSGKGLEWLAHIWWDDDKYYNPALKSRLTISKD | EKPDHLFTGLIGGINNRAPGVPARFSGSLIGDKAALTIT |
| | TSKNQVFLKIANVDTADTATYYCARIITTAWYFDVWG | GAQTEDEAIYFCALWYSNHFIFGSGTKVTVL (SEQ ID |
| | TGTTVTVSS (SEQ ID NO: 290) | NO: 388) |
| PDAB | QVQLQQPGAELVKPGTSVKMSCKASGYTFITYWITWV | QAVVTQESALTTSPGETVTLTCRSSTGAVTTSNYANWVQ |
| 200 | KQRPGHGLEWIGDIYPGSGSTNYNAKFRSKATLTVDT | EKPDHLFTGLIGGINNRAPGVPARFSGSLIGDKAALTIT |
| | SSSTAYMQFISLTSEDSAVYYCALKEIVPDYWGQGTT | GAQTEDEAIYFCALWYSNHLVFGGGTKLTVL (SEQ ID |
| | LTVSS (SEQ ID NO: 298) | NO: 391) |
| PDAB | QVQLQQPGAELVKPGASVKLSCKASGYTFTSYWMHWV | QAVVTQESALTTSPGETVTLTCRSSTGAVTTSNYANWVQ |
| 201 | KQRPGQGLEWIGMIHPNSGSTNYNEKFKSKATLTVDK | EKPDHLFTGLIGGTNNRAPGVPARFSGSLIGDKAALTIT |
| | SSSTAYMQLSSLISEDSAVYYCARSRGNYVWYFDVWG | GAQTEDEAIYFCALWYSNHSWVFGGGTKLTVL (SEQ |
| | TGTTVTVSS (SEQ ID NO: 294) | ID NO: 393) |
| PDAB | QVTLKESGPGILQPSQTLSLTCSFSGFSLSTFGMGVG | QAVVTQESALTTSPGETVTLTCRSSTGAVTTSNYANWVQ |
| 202 | WIRQPSGKGLEWLAHIWWDDDKYYNPALKSRLTISKD | EKPDHLFTGLIGGTNNRAPGVPARFSGSLIGDKAALTIT |
| | TSKNQVFLKIANVDTADTATYYCARMNPRNYEDYWGQ | GAQTEDEAIYFCALWYSNRWVFGGGTKLTVL (SEQ ID |
| | GTTLTVSS (SEQ ID NO: 295) | NO: 395) |
| PDAB | QVQLQQPGAELVKPGASVKVSCKASGYTFTSYWMHWV | QAVVTQESALTTSPGETVTLTCRSSTGAVTTSNYANWVQ |
| 203 | KQRPGQGLEWIGRIHPSDSDTNYNQKFKGKATLTVDK | EKPDHLFTGLIGGTNNRAPGVPARFSGSLIGDKAALTIT |
| | SSSTAYMQLSSLTSEDSAVYYCAIRDWDLDYWGQGTT | GAQTEDEAIYFCALWYSNRWVFGGGTKLTVL (SEQ ID |
| | LTVSS (SEQ ID NO: 296) | NQ : 395 |
| PDAB | QVTLKESGPGILQPSQTLSLTCSFSGFSLSTFGMGVG | QAVVTQESALTTSPGETVTLTCRSSTGAVTTSNYANWVQ |
| 204 | WIRQPSGKGLEWLAHIWWDDDKYYNPALKSRLTISKD | EKPDHLFTGLIGGTNNRAPGVPARFSGSLIGDKAALTIT |
| | TSKNQVFLKIANVDTADTATYYCARIPTRYWYFDVWG | GAQTEDEAIYFCALWYSTHYVFGGGTKVTVL (SEQ ID |
| | TGTTVTVSS (SEQ ID NO: 299) | NO: 397) |
| PDAB | QAYLQQSGAELVRPGASVKMSCKASGYTFTSYNMHWV | QAVVTQESALTTSPGETVTLTCRSSTGAVTTSNYANWVQ |
| 205 | KQTPRQGLEWIGAIYPGNGDTSYNQKFKGKATLTVDK | EKPDHLFTGLIGGINNRAPGVPARFSGSLIGDKAALTIT |
| | SSSTAYMQLSSLTSEDSAVYFCARSRDYDGLYAMDYW | GAQTEDEAIYFCALWYSTHYVEGGGTKVTVL (SEQ ID |
| | GQGTSVTVSS (SEQ ID NO: 300) | NO: 397) |
| PDAB | QVTLKESGPGILQPSQTLSLTCSFSGFSLSTFGMGVG | QAVVTQESALTTSPGGTVILTCRSSTGAVTTSNYANWVQ |
| 206 | WIRQPSGKGLEWLAHIWWDDDKYYNPALKSRLTISKD | EKPDHLFTGLIGGTSNRAPGVPVRFSGSLIGDKAALTIT |
| | TSKNQVFLKIANVDTADTATYYCARKWNYWYFDVWGT | GAQTEDDAMYFCALWYSTHYVEGGGTKVTVL (SEQ ID |
| | GTTVTVSS (SEQ ID NO: 301) | NO: 398) |
| PDAB | QVTLKESGPGILQPSQTLSLTCSFSGFSLSTFGMGVG | DIQMTQSPASLSASVGETVTITCGASENIYGGLNWYQRK |
| 207 | WIRQPSGKGLEWLAHIWWDDDKYYNPALKSRLTISKD | QGKSPQLLIYGATNLADGMSSRESGSGSGRQYSLKIRSL |
| | TSKNQVFLKIANVDTADTATYYCARKWNYWYFDVWGT | HPDDVATYYCQNVLSIPYTFGGGTKLEIK (SEQ ID |
| | GTTVTVSS (SEQ ID NO: 301) | NO: 399) |
| PDAB | QVQLQQPGAELVKPGASVKMSCKASGDTFTSYWITWV | QAVVTQESALTTSPGGTVILTCRSSTGAVTTSNYANWVQ |
| 208 | KQRPGQGLEWIGDIYPGSGSTNYNEKFKSKATLTVDT | EKPDHLFTGLIGGTSNRAPGVPVRFSGSLIGDKAALTIT |
| | SSSTAYMQLSSLISEDSAVYYCARKWNYWYFDVWGTG | GAQTEDDAMYFCALWYSTHYVFGGGTKVTVL (SEQ ID |
| | TTVTVSS (SEQ ID NO: 302) | NO: 398) |
| PDAB | QVQLQQPGAELVKPGASVKMSCKASGDTFTSYWITWV | DIQMTQSPASLSASVGETVTITCGASENIYGGLNWYQRK |
| 209 | KQRPGQGLEWIGDIYPGSGSTNYNEKFKSKATLTVDT | QGKSPQLLIYGATNLADGMSSRFSGSGSGRQYSLKIRSL |
| | SSSTAYMQLSSLISEDSAVYYCARKWNYWYFDVWGTG | HPDDVATYYCQNVLSIPYTFGGGTKLEIK (SEQ ID |
| | TTVTVSS (SEQ ID NO: 302) | NO: 399) |
| PDAB | QVQLQQPGAELVKPGASVKMSCKASGDTFTSYWITWV | QAVVTQESALTTSPGGTVILTCRSSTGAVTTSNYANWVQ |
| 210 | KQRPGQGLEWIGDIYPGSGSTNYNEKFKSKATLTVDT | EKPDHLFTGLIGGTSNRAPGVPVRFSGSLIGDKAALTIT |
| | SSSTAYMQLSSLISEDSAVYYCARRYYHGSSWYFDVW | GAQTEDDAMYFCALWYSTHYVEGGGTKVTVL (SEQ ID |
| | GTGTTVTVSS (SEQ ID NO: 303) | NO: 398) |
| PDAB | QVQLQQPGAELVKPGASVKMSCKASGDTFTSYWITWV | DIQMTQSPASLSASVGETVTITCGASENIYGGLNWYQRK |
| 211 | KQRPGQGLEWIGDIYPGSGSTNYNEKFKSKATLTVDT | QGKSPQLLIYGATNLADGMSSRFSGSGSGRQYSLKIRSL |
| | SSSTAYMQLSSLISEDSAVYYCARRYYHGSSWYFDVW | HPDDVATYYCQNVLSIPYTFGGGTKLEIK (SEQ ID |
| | GTGTTVTVSS (SEQ ID NO: 303) | NO: 399) |
| PDAB | QIQFVQSGPELKKPGETVKISCKASVYTFTEYPIHWV | DIVLTQSPASLAVSLGQRATISCRASESVDNYGISEMKW |
| 212 | KQAPGKGFKWMGCINTYSGEPTYADDFKRRFAFSLET | FQQKPGQSPKLLIYAASNQGSGVPARFSGSGSGTDFSLN |
| | SASTAYLQINNLKNEDTATYFCARCYGYDVFDYWGQG | IHPMEEDDTAMYFCQQSKEVPRTFGGGTKLEVK (SEQ |
| | TTLTVSS (SEQ ID NO: 304) | ID NO: 400) |
| PDAB | QVQLQQSGAELVRPGASVTLSCKASGYTFTDYEMHWV | DIVLTQSPASLAVSLGQRATISCRASESVDNYGISEMKW |
| 213 | KQTPVHGLEWIGTIDPETGGTAYNQKFKGKAILTADK | FQQKPGQSPKLLIYAASNQGSGVPARFSGSGSGTDFSLN |
| | SSSTAYMVLRSLTSEDSAVYYCTREGYGSPYYFDYWG | IHPMEEDDTAMYFCQQSKEVPRTFGGGTKLEVK (SEQ |
| | QGTTLTVSS (SEQ ID NO: 305) | ID NO: 400) |
| PDAB | QVTLKESGPGILQPSQTLSLTCSFSGFSLSTFGMGVG | DIVMTQSHKFMSTSVGDRVSITCKASQDVSTAVAWYQQK |
| 214 | WIRQPSGKGLEWLAHIWWDDDKYYNPALKSRLTISKD | PGQSPKLLIYSASYRYTGVPDRFTGSGSGTDFTFTISSV |
| | TSKNQVFLKIANVDTADTATYYCARIAEGRWYFDVWG | QAEDLAVYYCQQHYSTPYTFGGGTKLEIK (SEQ ID |
| | TGTTVTVSS (SEQ ID NO: 306) | NO: 401) |
| PDAB | QVQLQQPGAELVMPGASVKLSCKASGYTFTSYWMHWV | DIQMTQTTSSLSVSLGDRVTISCSASQVITNYLNWYQQK |
| 215 | KQRPGQGLEWIGEIDPSDSYTNYNQKFKGKSTLTVDK | PDGTIKLLIYYTSSLHSGVPSRFSGSGSGTDYSLTISNL |
| | SSSTAYMQLSSLISEDSAVYYCARSPEDYWGQGTTLT | EPEDIATYFCQQYDKLPWTFGGGTKLEIK (SEQ ID |
| | VSS (SEQ ID NO: 307) | NO: 402) |
| PDAB | EVQLQQSGPELVKPGASVKIPCKASGYTFTDYNMDWV | DIQMTQTTSSLSVSLGDRVTISCSASQVITNYLNWYQQK |
| 216 | KQSHGKSLEWIGDINPNNGGTIYNQKFKGKATLTVDK | PDGTIKLLIYYTSSLHSGVPSRFSGSGSGTDYSLTISNL |
| | SSSTAYMELRSLTSEDTGVYYCVTSIYYDSAWFGYWG | EPEDIATYFCQQYDKLPWTFGGGTKLEIK (SEQ ID |
| | QGTLVTVSA (SEQ ID NO: 308) | NO: 402) |
| PDAB | QIQLVQSGPELKKPGETVKISCKASGYTFTTYGMSWV | QAVVTQESALTTSPGETVTLTCRSSTGAVTTSNYANWVQ |
| 217 | KQAPGKGLKWMGWINTYSGVPAYAADFKGRFAFSLET | EKPDHLFTGLIGGTNNRAPGVPARFSGSLIGDKAALTIT |
| | SASTAYLQINTLKNEDTATYFCARGGNPYWGQGTLVT | GAQTEDEAIYFCALWYSNQFIFGSGTKVTVL (SEQ ID |
| | VSA (SEQ ID NO: 309) | NO: 403) |
| PDAB | QVTLKESGPGILQPSQTLSLTCSFSGFSLSTFGMGVG | QAVVIQESALTTSPGETVTLTCRSSTGAVTTSNYANWVQ |
| 218 | WIRQPSGKGLEWLAHIWWDDDKYYNPALKSRLTISKD | EKPDHLFTGLIGGTNNRAPGVPARFSGSLIGDKAALTIT |
| | TSKNQVFLKIANVDTADTATYYCARIRGTWWYFDVWG | GAQTEDEAIYFCALWYSNQFIFGSGTKVTVL (SEQ ID |
| | TGTTVTVSS (SEQ ID NO: 310) | NO: 403) |
| PDAB | EVQLQQSGPELVKPGASVKISCKASGYTFTDYYMNWV | ENVLTQSQAIMSASPGEKVTMTCRASSSVSSSYLHWYQQ |
| 219 | KQSHGKSLEWIGDINPNNGGTSYNQKFKGKATLTVDK | KSGASPKLWIYSTSNLASGVPARFSGSGSGTSYSLTISS |
| | SSSTAYMELRSLTSEDSAVYYCARRGGSSSYWYFDVW | VEAEDAATYYCQQYSGYPLTFGAGTKLELK (SEQ ID |
| | GTGTTVTVSS (SEQ ID NO: 311) | NO: 404) |
| PDAB | EVQLQQSGPELVKPGASVKISCKASGYTFTDYYMNWV | DIVLTQSPASLAVSLGQRATISCRASQSVSTSSYSYMHW |
| 220 | KQSHGKSLEWIGDINPNNGGTSYNQKFKGKATLTVDK | YQQKPGQPPKLLIKYASNLESGVPARFSGSGSGTDFTLN |
| | SSSTAYMELRSLTSEDSAVYYCARRGGSSSYWYFDVW | IHPVEEEDTATYYCQHSWEIPPTFGSGTKLEIK (SEQ |
| | GTGTTVTVSS (SEQ ID NO: 311) | ID NO: 405) |
| PDAB | EVQLQQSGPELVKPGASVKISCKASGYTFTDYYMNWV | DIQMTQSPASQSASLGESVTITCLASQTIGTWLAWYQQK |
| 221 | KQSHGKSLEWIGDINPNNGGTSYNQKFKGKATLTVDK | PGKSPQLLIYAATSLADGVPSRFSGSGSGTKFSFKISSL |
| | SSSTAYMELRSLTSEDSAVYYCARRGGSSSYWYFDVW | QAEDFVSYYCQQFYSTPWTFGGGTKLEIK (SEQ ID |
| | GTGTTVTVSS (SEQ ID NO: 311) | NO: 406) |
| PDAB | EVQLQQSGPELVKPGASVKISCKASGYTFTDYYMNWV | DIQMTQSPASQSASLGESVTITCLASQTIGTWLAWYQQK |
| 222 | KQSHGKSLEWIGDINPNNGGTSYNQKFKGKATLTVDK | PGKSPQLLIYAATSLADGVPSRFSGSGSGTKFSFKISSL |
| | SSSTAYMELRSLTSEDSAVYYCARRGGSSSYWYFDVW | QAEDFESYYCQQFYSTPWTFGGGTKLEIK (SEQ ID |
| | GTGTTVTVSS (SEQ ID NO: 311) | NO: 407) |
| PDAB | QVTLKESGPGILQPSQTLSLTCSFSGFSLSTFGMGVG | DIVLTQSPASLAVSLGQRATISCRASQSVSTSSYSYMHW |
| 223 | WIRQPSGKGLEWLAHIWWDDDKYYNPALKSRLTISKD | YQQKPGQPPKLLIKYASNLESGVPARFSGSGSGTDFTLN |
| | TSKNQVFLKIANVDTADTATYYCARKVGRPLWYFDVW | IHPVEEEDTATYYCQHSWEIPYTFGGGTKLEIK (SEQ |
| | GAGTTVTVSS (SEQ ID NO: 312) | ID NO: 408) |
| PDAB | QVTLKESGPGILQPSQTLSLTCSFSGFSLSTFGMGVG | QAVVTQESALTTSPGGTVILTCRSSTGAVTTSYYANWVQ |
| 224 | WIRQPSGKGLEWLAHIWWDDDKYYNPALKSRLTISKD | EKPDHLFTGLIGGTSNRAPGVPVRFSGSLIGDKAALTIT |
| | TSKNQVFLKIANVDTADTATYYCARKVGRPLWYFDVW | GAQTEDDAMYFCALWYSTHYVFGGGTKVTVL (SEQ ID |
| | GAGTTVTVSS (SEQ ID NO: 312) | NO: 409) |
| PDAB | QVQLKESGPGLVAPSQSLSITCTVSGFSLTSYAISWV | DIVLTQSPASLAVSLGQRATISCRASQSVSTSSYSYMHW |
| 225 | RQPPGKGLEWLGVIWTGGGTNYNSALKSRLSISKDNS | YQQKPGQPPKLLIKYASNLESGVPARFSGSGSGTDFTLN |
| | KSQVFLKMNSLQTDDTARYYCASSKGSYYAMDYWGQG | IHPVEEEDTATYYCQHSWEIPYTFGGGTKLEIK (SEQ |
| | TSVTVSS (SEQ ID NO: 313) | ID NO: 408) |
| PDAB | QVQLKESGPGLVAPSQSLSITCTVSGFSLTSYAISWV | QAVVTQESALTTSPGGTVILTCRSSTGAVTTSYYANWVQ |
| 226 | RQPPGKGLEWLGVIWTGGGTNYNSALKSRLSISKDNS | EKPDHLFTGLIGGTSNRAPGVPVRESGSLIGDKAALTIT |
| | KSQVFLKMNSLQTDDTARYYCASSKGSYYAMDYWGQG | GAQTEDDAMYFCALWYSTHYVFGGGTKVTVL (SEQ ID |
| | TSVTVSS (SEQ ID NO: 313) | NO: 409) |
| PDAB | QIQLVQSGPELKKPGATVKISCKASGFTFTTYGMSWV | QIVLTQSPTIMSASPGEKVTMTCSASLSLSSMFWYQQKP |
| 227 | KQAPGKGFKWMGWLNTYSGVPTYADDFKGRFAFSLET | GSSPRLLIYDTSKLASGVPVRFSGSGSGTSYSLTISRME |
| | SASTAYLQINNLKNEDTATYFCARGGYPYWGRGTTLT | AEDGATYYCQQWSSFPFTFGSGTKLEIK (SEQ ID |
| | VSS (SEQ ID NO: 314) | NO: 410) |
| PDAB | QIQLVQSGPELKKPGATVKISCKASGFTFTTYGMSWV | DIKMTQSPSSMYASLGERVTITCKASQDINSYLSWFQQK |
| 228 | KQAPGKGFKWMGWLNTYSGVPTYADDFKGRFAFSLET | PGKSPKTLIYRANRLVDGVPSRFSGSGSGQDYSLTISSL |
| | SASTAYLQINNLKNEDTATYFCARGGYPYWGRGTTLT | EYEDMGIYYCLQYDEFPYTFGGGTKLEIK (SEQ ID |
| | VSS (SEQ ID NO: 314) | NO: 411) |
| PDAB | EVQLQQSGAELVKPGASVKLSCTASGFNIKDYYMHWV | QIVLTQSPTIMSASPGEKVTMTCSASLSLSSMFWYQQKP |
| 229 | KQRTEQGLEWIGRIDPEDGETKYAPKFQGKATITADT | GSSPRLLIYDTSKLASGVPVRFSGSGSGTSYSLTISRME |
| | SSNTAYLQLSSLTSEDTAVYYCGRDGYYDVYFDYWGQ | AEDGATYYCQQWSSFPFTFGSGTKLEIK (SEQ ID |
| | GTTLTVSS (SEQ ID NO: 315) | NO: 410) |
| PDAB | EVQLQQSGAELVKPGASVKLSCTASGFNIKDYYMHWV | DIKMTQSPSSMYASLGERVTITCKASQDINSYLSWFQQK |
| 230 | KQRTEQGLEWIGRIDPEDGETKYAPKFQGKATITADT | PGKSPKTLIYRANRLVDGVPSRFSGSGSGQDYSLTISSL |
| | SSNTAYLQLSSLTSEDTAVYYCGRDGYYDVYFDYWGQ | EYEDMGIYYCLQYDEFPYTFGGGTKLEIK (SEQ ID |
| | GTTLTVSS (SEQ ID NO: 315) | NO: 411) |
| PDAB | QVQLQQSGAELVKPGASVKISCKTSGYIFSNYWMNWV | QIVLTQSPTIMSASPGEKVTMTCSASLSLSSMFWYQQKP |
| 231 | KQRPGKGLEWIGQIYPGDGDTNYNGDFKGKATLTADK | GSSPRLLIYDTSKLASGVPVRFSGSGSGTSYSLTISRME |
| | SSSTAYIYLNSLTSEDSAVYFCARGPYWGLGTLVTVS | AEDGATYYCQQWSSFPFTFGSGTKLEIK (SEQ ID |
| | A (SEQ ID NO: 316) | NO: 410) |
| PDAB | QVQLQQSGAELVKPGASVKISCKTSGYIFSNYWMNWV | DIKMTQSPSSMYASLGERVTITCKASQDINSYLSWFQQK |
| 232 | KQRPGKGLEWIGQIYPGDGDTNYNGDFKGKATLTADK | PGKSPKTLIYRANRLVDGVPSRFSGSGSGQDYSLTISSL |
| | SSSTAYIYLNSLTSEDSAVYFCARGPYWGLGTLVTVS | EYEDMGIYYCLQYDEFPYTFGGGTKLEIK (SEQ ID |
| | A (SEQ ID NO: 316) | NO: 411) |
| PDAB | QVTLKESGPGILQPSQTLSLTCSFSGFSLSTFGMGVG | QAVVTQESALTTSPGETVTLTCRSSTGAVTTSNYANWVQ |
| 233 | WIRQPSGKGLEWLAHIWWDDDKYYNPALKSRLTISKD | EKPDHLFTGLIGGTNNRAPGVPARFSGSLIGDKAALTIT |
| | TSKNQVFLKIANVDTADTATYYCARIQSFYWYFDVWG | GAQTEDEAIYFCALWYSNHYIFGSGTKVTVL (SEQ ID |
| | TGTTVTVSS (SEQ ID NO: 317) | NO: 412) |
| PDAB | EVQLQQSGAELVKPGASVKLSCTASGFNIKDYYMHWV | QAVVTQESALTTSPGETVTLTCRSSTGAVTTSNYANWVQ |
| 234 | KQRTEQGLEWIGRIDPEDGETKYAPKFQGKTTITADT | EKPDHLFTGLIGGTNNRAPGVPARFSGSLIGDKAALTIT |
| | SSNTAYLQLSSLTSEDTAVYYCGRDGYYDVYFDYWGQ | GAQTEDEAIYFCALWYSNHYIFGSGTKVTVL (SEQ ID |
| | GTTLTVSS (SEQ ID NO: 318) | NO: 412) |
| PDAB | QIQLVQSGPELKKPGETVKISCKASGYTFTTYGMSWV | DVVMTQTPLTLSVTIGQPASISCKSSQSLLDSDGKTYLN |
| 235 | KQAPGKGLKWMGWINTYSGLPTYADDFKGRFAFSLET | WLLQRPGQSPKRLIYLVSKLDSGVPDRFTGSGSGTDFTL |
| | SASTAYLQINNLKNEDTATYFCARGGYDYGAAYWGQG | KISRVEAEDLGVYYCWQGTHEPHTFGAGTKLELK (SEQ |
| | TLVTVSA (SEQ ID NO: 319) | ID NO: 413) |
| PDAB | QIQLVQSGPELKKPGETVKISCKASGYTFTTYGMSWV | QAVVTQESALTTSPGETVTLTCRSSTGAVTTSNYANWVQ |
| 236 | KQAPGKGLKWMGWINTYSGLPTYADDFKGRFAFSLET | EKPDHLFTGLIGGTNNRAPGVPARFSGSLIGDKAALTIT |
| | SASTAYLQINNLKNEDTATYFCARGGYDYGAAYWGQG | GAQTEDEAIYFCALWYSNHLVEGGGTKLTVL (SEQ ID |
| | TLVTVSA (SEQ ID NO: 319) | NO: 391) |
| PDAB | QVTLKESGPGILQPSQTLSLICSFSGFSLSTFGMGVG | DVVMTQTPLTLSVTIGQPASISCKSSQSLLDSDGKTYLN |
| 237 | WIRQPSGKGLEWLTHIWWDDDKYYNPALKSRLTISKD | WLLQRPGQSPKRLIYLVSKLDSGVPDRFTGSGSGTDFTL |
| | TSKNQVFLKIANVDTADTATYYCARVRMSHWYFDVWA | KISRVEAEDLGVYYCWQGTHEPHTFGAGTKLELK (SEQ |
| | TGTTVTVSS (SEQ ID NO: 320) | ID NO: 413) |
| PDAB | QVTLKESGPGILQPSQTLSLTCSFSGFSLSTFGMGVG | QAVVTQESALTTSPGETVTLTCRSSTGAVTTSNYANWVQ |
| 238 | WIRQPSGKGLEWLTHIWWDDDKYYNPALKSRLTISKD | EKPDHLFTGLIGGINNRAPGVPARFSGSLIGDKAALTIT |
| | TSKNQVFLKIANVDTADTATYYCARVRMSHWYFDVWA | GAQTEDEAIYFCALWYSNHLVEGGGTKLIVL (SEQ ID |
| | TGTTVTVSS (SEQ ID NO: 320) | NO: 391) |
| PDAB | QVQLQQSGAELMKPGASVKLSCKATGYTFTGYWIEWV | DVVMTQTPLTLSVTIGQPASISCKSSQSLLDSDGKTYLN |
| 239 | KQRPGHGLEWIGEILPGSGSTNYNEKFKGKATFTADT | WLLQRPGQSPKRLIYLVSKLDSGVPDRFTGSGSGTDFTL |
| | SSNTAYMQLSSLTTEDSAIHYCARKEDGYYLYYFDYW | KISRVEAEDLGVYYCWQGTHEPHTFGAGTKLELK (SEQ |
| | GQGTTLTVSS (SEQ ID NO: 321) | ID NO: 413) |
| PDAB | QVQLQQSGAELMKPGASVKLSCKATGYTFTGYWIEWV | QAVVTQESALTTSPGETVTLTCRSSTGAVTTSNYANWVQ |
| 240 | KQRPGHGLEWIGEILPGSGSTNYNEKFKGKATFTADT | EKPDHLFTGLIGGTNNRAPGVPARFSGSLIGDKAALTIT |
| | SSNTAYMQLSSLTTEDSAIHYCARKEDGYYLYYFDYW | GAQTEDEAIYFCALWYSNHLVEGGGTKLTVL (SEQ ID |
| | GQGTTLTVSS (SEQ ID NO: 321) | NO: 391) |
| PDAB | QVQLQQSGAVLMKPGASVKLSCKATGYTITGSWIAWL | DIQMTQTTSSLSASLGDRVTISCRASQDISNYLNWYQQK |
| 241 | KQRPGHGLEWIGEILPGSGRTNLNEDFKGKATFTADT | PDGTVKLLIYYTSRLHSGVPSRFSGSGSGTDYSLTIRNL |
| | SSNTVFIQLSSLTTEDSAIYYCYNGSAFDYWGQGTTL | DQEDIATYFCQQDKTFPWTFGGGTKLEIK (SEQ ID |
| | TVSS (SEQ ID NO: 322) | NO: 414) |
| PDAB | EVQLLESGGGLVQPGGSLRLSCAASGFTFSDYYMSWV | QSVLTQPPSASGAPGQRVTISCSGSYSNIGESYVSWYQQ |
| 242 | RQAPGKGLEWVSVISNSGGSTYYTDSVKGRFTISRDN | LPGTAPKLLIYLNSQRPSGVPDRESGSKSGTSASLAISG |
| | SKNTLYLQMNSLRAEDTAVYYCTKDIGMTYFDYWGQG | LQSEDEADYYCAAWDDLNVWVFGGGTKLIVL (SEQ ID |
| | TLVTVSS (SEQ ID NO: 530) | NO: 531) |
| PDAB | EVQLLESGGGLVQPGGSLRLSCAASGFTFSDYYMSWV | QSVLTQPPSASGAPGQRVTISCSGSYSNIGGSYVSWYQQ |
| 243 | RQAPGKGLEWVSVISNSGGSTYYTDSVKGRFTISRDN | LPGTAPKLLIYLNSQRPSGVPDRFSGSKSGTSASLAISG |
| | SKNTLYLQMNSLRAEDTAVYYCTKDIGMTYFDYWGQG | LQSEDEADYYCAAWDDLNVWVFGGGTKLTVL (SEQ ID |
| | TLVTVSS (SEQ ID NO: 530) | NO: 532) |
| PDAB | EVQLLESGGGLVQPGGSLRLSCAASGFTFSDYYMSWV | QSVLTQPPSASGAPGQRVTISCSGSYSNIGLSYVSWYQQ |
| 244 | RQAPGKGLEWVSVISNSGGSTYYTDSVKGRFTISRDN | LPGTAPKLLIYLNSQRPSGVPDRFSGSKSGTSASLAISG |
| | SKNTLYLQMNSLRAEDTAVYYCTKDIGMTYFDYWGQG | LQSEDEADYYCAAWDDLNVWVFGGGTKLTVL (SEQ ID |
| | TLVTVSS (SEQ ID NO: 530) | NO: 533) |
| PDAB | EVQLLESGGGLVQPGGSLRLSCAASGFTFSDYYMSWV | QSVLTQPPSASGAPGQRVTISCSGSYSNIGQSYVSWYQQ |
| 245 | RQAPGKGLEWVSVISNSGGSTYYTDSVKGRFTISRDN | LPGTAPKLLIYLNSQRPSGVPDRFSGSKSGTSASLAISG |
| | SKNTLYLQMNSLRAEDTAVYYCTKDIGMTYFDYWGQG | LQSEDEADYYCAAWDDLNVWVFGGGTKLTVL (SEQ ID |
| | TLVTVSS (SEQ ID NO: 530) | NO: 534) |
| PDAB | EVQLLESGGGLVQPGGSLRLSCAASGFTFSDYYMSWV | QSVLTQPPSASGAPGQRVTISCSGSYSNIGSSYVSWYQQ |
| 246 | RQAPGKGLEWVSVISNSGGSTYYTDSVKGRFTISRDN | LPGTAPKLLIYLNSQRPSGVPDRFSGSKSGTSASLAISG |
| | SKNTLYLQMNSLRAEDTAVYYCTKDIGMTYFDYWGQG | LQSEDEADYYCAAWDDLNVWVFGGGTKLTVL (SEQ ID |
| | TLVTVSS (SEQ ID NO: 530) | NO: 535) |
| PDAB | EVQLLESGGGLVQPGGSLRLSCAASGFTFSDYYMSWV | QSVLTQPPSASGAPGQRVTISCSGSYSNIGNDYVSWYQQ |
| 247 | RQAPGKGLEWVSVISNSGGSTYYTDSVKGRFTISRDN | LPGTAPKLLIYLNSQRPSGVPDRESGSKSGTSASLAISG |
| | SKNTLYLQMNSLRAEDTAVYYCTKDIGMTYFDYWGQG | LQSEDEADYYCAAWDDLNVWVFGGGTKLTVL (SEQ ID |
| | TLVTVSS (SEQ ID NO: 530) | NO: 536) |
| PDAB | EVQLLESGGGLVQPGGSLRLSCAASGFTFSDYYMSWV | QSVLTQPPSASGAPGQRVTISCSGSYSNIGNQYVSWYQQ |
| 248 | RQAPGKGLEWVSVISNSGGSTYYTDSVKGRFTISRDN | LPGTAPKLLIYLNSQRPSGVPDRFSGSKSGTSASLAISG |
| | SKNTLYLQMNSLRAEDTAVYYCTKDIGMTYFDYWGQG | LQSEDEADYYCAAWDDLNVWVFGGGTKLTVL (SEQ ID |
| | TLVTVSS (SEQ ID NO: 530) | NO: 537) |
| PDAB | QVTLKESGPTLVKPTQTLTLTCTFSGFSLSTFGMGVG | QAVVTQEPSLTVSPGGTVTLTCRSSTGAVTTSNYANWVQ |
| 249 | WIRQPPGKALEWLAHIWWDDDKYYNPALKSRLTITKD | QKPGQAFRGLIGGINNRAPGVPDRFSGSILGNKAALTIT |
| | TSKNQVVLTMTNMDPVDTATYYCARIITTAWYFDVWG | GAQADDESIYFCALWYSNHFIFGSGTKVTVL (SEQ ID |
| | TGTTVTVSS (SEQ ID NO: 538) | NO: 539) |
| PDAB | QVTLKESGPTLVKPTQTLTLTCSFSGFSLSTFGMGVG | QAVVTQEPSLTVSPGGTVTLTCRSSTGAVTTSNYANWVQ |
| 250 | WIRQPPGKGLEWIGHIWWDDDKYYNPALKSRLTITKD | QKPGQAFRGLIGGINNRAPGVPDRFSGSILGNKAALTIT |
| | TSKNQVVLTMTNMDPVDTATYYCARIITTAWYFDVWG | GAQADDESIYFCALWYSNHFIFGSGTKVTVL (SEQ ID |
| | TGTTVTVSS (SEQ ID NO: 540) | NO: 539) |
| PDAB | QVTLKESGPTLVKPTQTLTLTCSFSGFSLSTEGMGVG | QAVVTQEPSLTVSPGGTVTLTCRSSTGAVTTSNYANWVQ |
| 251 | WIRQPPGKGLEWIGHIWWDDDKYYNPALKSRLTITKD | QKPGQAFRGLIGGINNRAPGVPDRESGSLIGDKAALTIT |
| | TSKNQVVLTMTNMDPVDTATYYCARIITTAWYFDVWG | GAQADDESDYYCALWYSNHFIFGSGTKVTVL (SEQ ID |
| | TGTTVTVSS (SEQ ID NO: 540) | NO: 541) |
| PDAB | QVTLKESGPTLVKPTQTLTLTCTFSGFSLSTFGMGVG | QAVVTQEPSLTVSPGGTVTLTCRSSTGAVTTSNYANWVQ |
| 252 | WIRQPPGKALEWLAHIWWDDDKYYNPALKSRLTITKD | QKPGQAFRGLIGGTNNRAPGVPDRFSGSILGNKAALTIT |
| | TSKNQVVLTMTNMDPVDTATYYCARIITTAWYFDVWG | GAQADDESDYYCALWYSNHFIFGSGTKVTVL (SEQ ID |
| | TGTTVTVSS (SEQ ID NO: 538) | NO: 542) |
| PDAB | QVTLKESGPTLVKPTQTLTLTCSFSGFSLSTFGMGVG | QAVVTQEPSLTVSPGGTVTLTCRSSTGAVTTSNYANWVQ |
| 253 | WIRQPPGKGLEWIGHIWWDDDKYYNPALKSRLTITKD | QKPGQAFRGLIGGINNRAPGVPDRESGSILGNKAALTIT |
| | TSKNQVVLTMTNMDPVDTATYYCARIITTAWYFDVWG | GAQADDESDYYCALWYSNHFIFGSGTKVTVL (SEQ ID |
| | TGTTVTVSS (SEQ ID NO: 540) | NO: 542) |
| PDAB | EVQLLESGGGLVQPGGSLRLSCAASGFTFSDYYMSWV | QSVLTQPPSASGAPGQRVTISCSGSYSNIGNSYVSWYQQ |
| 254 | RQAPGKGLEWVSVISNSGGSTYYTDSVKGRFTISRDN | LPGTAPKLLIYLNSQRPSGVPDRESGSKSGTSASLAISG |
| | SKNTLYLQMNSLRAEDTAVYYCTKDIGMTYFDYWGQG | LQSEDEADYYCAAWDDLNVWVFGGGTKLTVL (SEQ ID |
| | TLVTVSS (SEQ ID NO: 530) | NO: 344) |

In some embodiments, an anti-PD-1 antibody, or an antigen-binding fragment thereof, comprises a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 530, 538, and 540. In some embodiments, the anti-PD-1 antibody or one that has percent identity to the reference VH sequence set forth above comprises the HCDRs for the clone number as provided for herein.

In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 130. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 131. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 132. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 133. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 134. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 135. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 136. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 137. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 138. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 139. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 140. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 141. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 142. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 143. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 144. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 145. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 146. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 147. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 148. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 149. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 150. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 151. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 152. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 153. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 154. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 155. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 156. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 157. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 158. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 159. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 160. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 161. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 162. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 163. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 164. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 165. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 166. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 167. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 168. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 169. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 170. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 171. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 172. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 173. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 174. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 175. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 176. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 177. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 178. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 179. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 180. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 181. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 182. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 183. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 184. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 185. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 186. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 187. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 188. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 189. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 190. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 191. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 192. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 193. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 194. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 195. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 196. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 197. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 198. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 199. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 200. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 201. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 202. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 203. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 204. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 205. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 206. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 207. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 208. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 209. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 210. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 211. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 212. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 213. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 214. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 215. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 216. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 217. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 218. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 219. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 220. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 221. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 222. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 223. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 224. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 225. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 226. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 227. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 228. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 229. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 230. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 231. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 232. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 233. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 234. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 235. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 236. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 237. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 238. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 239. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 240. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 241. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 242. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 243. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 244. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 245. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 246. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 247. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 248. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 249. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 250. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 251. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 252. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 253. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 254. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 255. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 256. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 257. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 258. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 259. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 260. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 261. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 262. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 263. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 264. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 265. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 266. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 267. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 268. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 269. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 270. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 271. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 272. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 273. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 274. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 275. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 276. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 277. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 278. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 279. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 280. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 281. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 282. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 283. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 284. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 285. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 286. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 287. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 288. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 289. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 290. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 291. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 292. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 293. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 294. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 295. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 296. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 297. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 298. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 299. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 300. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 301. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 302. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 303. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 304. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 305. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 306. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 307. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 308. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 309. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 310. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 311. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 312. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 313. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 314. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 315. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 316. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 317. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 318. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 319. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 320. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 321. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 322. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 530. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 538. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 540.

For the percent identity claims provided in regards to the variable heavy chain domain, in some embodiments, the variant comprises the HCDRs as provided for in the reference sequence. The CDRs can be determined via KABAT, Chothia, or IMGT systems, which are known by one of skill in the art.

In some embodiments, an anti-PD-1 antibody, or an antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 531, 532, 533, 534, 535, 536, 537, 539, 541, and 542. In some embodiments, the variable light chains provided for herein may be combined with the different VH domains provided for herein as illustrated in the tables because, and without being bound by any particular theory, the light chain allows for flexibility in antigenic binding. This is illustrated in, for example, Table 5, which shows multiple antibodies sharing the same VL domain.

In some embodiments, an anti-PD-1 antibody, or an antigen-binding fragment thereof, comprises a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 530, 538, and 540; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 531, 532, 533, 534, 535, 536, 537, 539, 541, and 542.

In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 130; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 131; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 132; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 133; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 134; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 135; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 136; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 137; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 138; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 139; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 140; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 141; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 142; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 143; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 144; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 145; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 146; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 147; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 148; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 149; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 150; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 151; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 152; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 153; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 154; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 155; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 156; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 157; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 324. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 158; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 325. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 159; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 326. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 160; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 327. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 161; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 328. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 162; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 326. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 163; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 164; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 165; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 166; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 167; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 168; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 169; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 170; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 171; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 172; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 173; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 174; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 175; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 329. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 176; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 330. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 177; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 331. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 175; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 332. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 178; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 333. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 179; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 334. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 180; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 335. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 181; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 336. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 182; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 337. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 183; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 338. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 184; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 339. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 185; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 340. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 186; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 341. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 186; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 342. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 180; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 343. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 187; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 344. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 183; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 344. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 185; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 345. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 188; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 346. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 189; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 347. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 182; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 346. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 190; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 191; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 192; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 193; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 194; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 195; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 196; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 156; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 197; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 198; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 199; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 200; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 201; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 202; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 203; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 204; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 205; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 206; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 130; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 207; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 208; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 209; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 135; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 210; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 211; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 212; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 204; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 213; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 214; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 215; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 216; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 217; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 218; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 219; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 220; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 221; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 222; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 223; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 224; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 225; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 226; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 227; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 228; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 158; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 348. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 229; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 349. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 230; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 350. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 160; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 351. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 231; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 352. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 232; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 353. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 233; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 354. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 234; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 355. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 235; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 356. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 236; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 357. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 237; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 358. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 238; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 359. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 239; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 328. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 240; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 328. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 233; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 327. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 241; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 360. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 242; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 327. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 243; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 356. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 158; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 361. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 244; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 362. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 230; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 363. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 245; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 364. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 246; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 365. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 244; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 366. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 247; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 367. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 248; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 368. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 249; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 369. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 250; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 348. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 251; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 348. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 252; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 370. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 230; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 371. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 253; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 372. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 254; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 348. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 255; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 373. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 256; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 374. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 245; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 375. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 257; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 348. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 258; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 376. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 158; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 377. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 259; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 348. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 260; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 356. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 261; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 378. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 262; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 379. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 263; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 348. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 264; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 380. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 262; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 370. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 265; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 363. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 158; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 382. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 158; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 383. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 264; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 384. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 264; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 385. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 253; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 386. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 246; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 374. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 266; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 387. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 267; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 268; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 269; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 270; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 271; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 272; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 273; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 274; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 275; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 276; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 277; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 278; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 279; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 280; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 281; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 282; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 283; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 284; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 285; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 286; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 287; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 288; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 289; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 323. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 290; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 388. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 291; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 389. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 292; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 390. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 293; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 391. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 293; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 392. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 294; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 393. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 295; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 394. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 295; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 395. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 296; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 394. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 296; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 395. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 297; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 396. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 290; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 388. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 298; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 391. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 294; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 393. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 295; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 395. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 296; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 395. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 299; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 397. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 300; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 397. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 301; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 398. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 301; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 399. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 302; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 398. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 302; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 399. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 303; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 398. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 303; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 399. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 304; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 400. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 305; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 400. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 306; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 401. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 307; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 402. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 308; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 402. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 309; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 403. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 310; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 403. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 311; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 404. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 311; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 405. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 311; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 406. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 311; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 407. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 312; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 408. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 312; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 409. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 313; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 408. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 313; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 409. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 314; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 410. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 314; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 411. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 315; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 410. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 315; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 411. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 316; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 410. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 316; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 411. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 317; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 412. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 318; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 412. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 319; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 413. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 319; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 391. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 320; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 413. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 320; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 391. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 321; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 413. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 321; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 391. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 322; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 414. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 530; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 531. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 530; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 532. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 530; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 533. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 530; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 534. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 530; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 535. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 530; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 536. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 530; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 537. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 538; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 539. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 540; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 539. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 540; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 541. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 538; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 542. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 540; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 542. In some embodiments, an anti-PD-1 antibody, or an antigen-binding fragment thereof, comprises a variable heavy chain comprising the amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 530; and a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 344.

In some embodiments, an anti-PD-1 antibody, or an antigen-binding fragment thereof, comprises a variable heavy chain comprising an amino acid sequence selected from any one of SEQ ID NOs: 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 530, 538, and 540.

In some embodiments, an anti-PD-1 antibody, or an antigen-binding fragment thereof, comprises a variable heavy chain comprising the amino acid sequence of SEQ ID NO: 130. In some embodiments, an anti-PD-1 antibody, or an antigen-binding fragment thereof, comprises a variable heavy chain comprising the amino acid sequence of SEQ ID NO: 131. In some embodiments, an anti-PD-1 antibody, or an antigen-binding fragment thereof, comprises a variable heavy chain comprising the amino acid sequence of SEQ ID NO: 132. In some embodiments, an anti-PD-1 antibody, or an antigen-binding fragment thereof, comprises a variable heavy chain comprising the amino acid sequence of SEQ ID NO: 133. In some embodiments, an anti-PD-1 antibody, or an antigen-binding fragment thereof, comprises a variable heavy chain comprising the amino acid sequence of SEQ ID NO: 134. In some embodiments, an anti-PD-1 antibody, or an antigen-binding fragment thereof, comprises a variable heavy chain comprising the amino acid sequence of SEQ ID NO: 135. In some embodiments, an anti-PD-1 antibody, or an antigen-binding fragment thereof, comprises a variable heavy chain comprising the amino acid sequence of SEQ ID NO: 136. In some embodiments, an anti-PD-1 antibody, or an antigen-binding fragment thereof, comprises a variable heavy chain comprising the amino acid sequence of SEQ ID NO: 137. In some embodiments, an anti-PD-1 antibody, or an antigen-binding fragment thereof, comprises a variable heavy chain comprising the amino acid sequence of SEQ ID NO: 138. In some embodiments, an anti-PD-1 antibody, or an antigen-binding fragment thereof, comprises a variable heavy chain comprising the amino acid sequence of SEQ ID NO: 139. In some embodiments, an anti-PD-1 antibody, or an antigen-binding fragment thereof, comprises a variable heavy chain comprising the amino acid sequence of SEQ ID NO: 140. In some embodiments, an anti-PD-1 antibody, or an antigen-binding fragment thereof, comprises a variable heavy chain comprising the amino acid sequence of SEQ ID NO: 141. In some embodiments, an anti-PD-1 antibody, or an antigen-binding fragment thereof, comprises a variable heavy chain comprising the amino acid sequence of SEQ ID NO: 142. In some embodiments, an anti-PD-1 antibody, or an antigen-binding fragment thereof, comprises a variable heavy chain comprising the amino acid sequence of SEQ ID NO: 143. In some embodiments, an anti-PD-1 antibody, or an antigen-binding fragment thereof, comprises a variable heavy chain comprising the amino acid sequence of SEQ ID NO: 144. In some embodiments, an anti-PD-1 antibody, or an antigen-binding fragment thereof, comprises a variable heavy chain comprising the amino acid sequence of SEQ ID NO: 145. In some embodiments, an anti-PD-1 antibody, or an antigen-binding fragment thereof, comprises a variable heavy chain comprising the amino acid sequence of SEQ ID NO: 146. In some embodiments, an anti-PD-1 antibody, or an antigen-binding fragment thereof, comprises a variable heavy chain comprising the amino acid sequence of SEQ ID NO: 147. In some embodiments, an anti-PD-1 antibody, or an antigen-binding fragment thereof, comprises a variable heavy chain comprising the amino acid sequence of SEQ ID NO: 148. In some embodiments, an anti-PD-1 antibody, or an antigen-binding fragment thereof, comprises a variable heavy chain comprising the amino acid sequence of SEQ ID NO: 149. In some embodiments, an anti-PD-1 antibody, or an antigen-binding fragment thereof, comprises a variable heavy chain comprising the amino acid sequence of SEQ ID NO: 150. In some embodiments, an anti-PD-1 antibody, or an antigen-binding fragment thereof, comprises a variable heavy chain comprising the amino acid sequence of SEQ ID NO: 151. In some embodiments, an anti-PD-1 antibody, or an antigen-binding fragment thereof, comprises a variable heavy chain comprising the amino acid sequence of SEQ ID NO: 152. In some embodiments, an anti-PD-1 antibody, or an antigen-binding fragment thereof, comprises a variable heavy chain comprising the amino acid sequence of SEQ ID NO: 153. In some embodiments, an anti-PD-1 antibody, or an antigen-binding fragment thereof, comprises a variable heavy chain comprising the amino acid sequence of SEQ ID NO: 154. In some embodiments, an anti-PD-1 antibody, or an antigen-binding fragment thereof, comprises a variable heavy chain comprising the amino acid sequence of SEQ ID NO: 155. In some embodiments, an anti-PD-1 antibody, or an antigen-binding fragment thereof, comprises a variable heavy chain comprising the amino acid sequence of SEQ ID NO: 156. In some embodiments, an anti-PD-1 antibody, or an antigen-binding fragment thereof, comprises a variable heavy chain comprising the amino acid sequence of SEQ ID NO: 157. In some embodiments, an anti-PD-1 antibody, or an antigen-binding fragment thereof, comprises a variable heavy chain comprising the amino acid sequence of SEQ ID NO: 158. In some embodiments, an anti-PD-1 antibody, or an antigen-binding fragment thereof, comprises a variable heavy chain comprising the amino acid sequence of SEQ ID NO: 159. In some embodiments, an anti-PD-1 antibody, or an antigen-binding fragment thereof, comprises a variable heavy chain comprising the amino acid sequence of SEQ ID NO: 160. In some embodiments, an anti-PD-1 antibody, or an antigen-binding fragment thereof, comprises a variable heavy chain comprising the amino acid sequence of SEQ ID NO: 161. In some embodiments, an anti-PD-1 antibody, or an antigen-binding fragment thereof, comprises a variable heavy chain comprising the amino acid sequence of SEQ ID NO: 162. In some embodiments, an anti-PD-1 antibody, or an antigen-binding fragment thereof, comprises a variable heavy chain comprising the amino acid sequence of SEQ ID NO: 163. In some embodiments, an anti-PD-1 antibody, or an antigen-binding fragment thereof, comprises a variable heavy chain comprising the amino acid sequence of SEQ ID NO: 164. In some embodiments, an anti-PD-1 antibody, or an antigen-binding fragment thereof, comprises a variable heavy chain comprising the amino acid sequence of SEQ ID NO: 165. In some embodiments, an anti-PD-1 antibody, or an antigen-binding fragment thereof, comprises a variable heavy chain comprising the amino acid sequence of SEQ ID NO: 166. In some embodiments, an anti-PD-1 antibody, or an antigen-binding fragment thereof, comprises a variable heavy chain comprising the amino acid sequence of SEQ ID NO: 167. In some embodiments, an anti-PD-1 antibody, or an antigen-binding fragment thereof, comprises a variable heavy chain comprising the amino acid sequence of SEQ ID NO: 168. In some embodiments, an anti-PD-1 antibody, or an antigen-binding fragment thereof, comprises a variable heavy chain comprising the amino acid sequence of SEQ ID NO: 169. In some embodiments, an anti-PD-1 antibody, or an antigen-binding fragment thereof, comprises a variable heavy chain comprising the amino acid sequence of SEQ ID NO: 170. In some embodiments, an anti-PD-1 antibody, or an antigen-binding fragment thereof, comprises a variable heavy chain comprising the amino acid sequence of SEQ ID NO: 171. In some embodiments, an anti-PD-1 antibody, or an antigen-binding fragment thereof, comprises a variable heavy chain comprising the amino acid sequence of SEQ ID NO: 172. In some embodiments, an anti-PD-1 antibody, or an antigen-binding fragment thereof, comprises a variable heavy chain comprising the amino acid sequence of SEQ ID NO: 173. In some embodiments, an anti-PD-1 antibody, or an antigen-binding fragment thereof, comprises a variable heavy chain comprising the amino acid sequence of SEQ ID NO: 174. In some embodiments, an anti-PD-1 antibody, or an antigen-binding fragment thereof, comprises a variable heavy chain comprising the amino acid sequence of SEQ ID NO: 175. In some embodiments, an anti-PD-1 antibody, or an antigen-binding fragment thereof, comprises a variable heavy chain comprising the amino acid sequence of SEQ ID NO: 176. In some embodiments, an anti-PD-1 antibody, or an antigen-binding fragment thereof, comprises a variable heavy chain comprising the amino acid sequence of SEQ ID NO: 177. In some embodiments, an anti-PD-1 antibody, or an antigen-binding fragment thereof, comprises a variable heavy chain comprising the amino acid sequence of SEQ ID NO: 178. In some embodiments, an anti-PD-1 antibody, or an antigen-binding fragment thereof, comprises a variable heavy chain comprising the amino acid sequence of SEQ ID NO: 179. In some embodiments, an anti-PD-1 antibody, or an antigen-binding fragment thereof, comprises a variable heavy chain comprising the amino acid sequence of SEQ ID NO: 180. In some embodiments, an anti-PD-1 antibody, or an antigen-binding fragment thereof, comprises a variable heavy chain comprising the amino acid sequence of SEQ ID NO: 181. In some embodiments, an anti-PD-1 antibody, or an antigen-binding fragment thereof, comprises a variable heavy chain comprising the amino acid sequence of SEQ ID NO: 182. In some embodiments, an anti-PD-1 antibody, or an antigen-binding fragment thereof, comprises a variable heavy chain comprising the amino acid sequence of SEQ ID NO: 183. In some embodiments, an anti-PD-1 antibody, or an antigen-binding fragment thereof, comprises a variable heavy chain comprising the amino acid sequence of SEQ ID NO: 184. In some embodiments, an anti-PD-1 antibody, or an antigen-binding fragment thereof, comprises a variable heavy chain comprising the amino acid sequence of SEQ ID NO: 185. In some embodiments, an anti-PD-1 antibody, or an antigen-binding fragment thereof, comprises a variable heavy chain comprising the amino acid sequence of SEQ ID NO: 186. In some embodiments, an anti-PD-1 antibody, or an antigen-binding fragment thereof, comprises a variable heavy chain comprising the amino acid sequence of SEQ ID NO: 187. In some embodiments, an anti-PD-1 antibody, or an antigen-binding fragment thereof, comprises a variable heavy chain comprising the amino acid sequence of SEQ ID NO: 188. In some embodiments, an anti-PD-1 antibody, or an antigen-binding fragment thereof, comprises a variable heavy chain comprising the amino acid sequence of SEQ ID NO: 189. In some embodiments, an anti-PD-1 antibody, or an antigen-binding fragment thereof, comprises a variable heavy chain comprising the amino acid sequence of SEQ ID NO: 190. In some embodiments, an anti-PD-1 antibody, or an antigen-binding fragment thereof, comprises a variable heavy chain comprising the amino acid sequence of SEQ ID NO: 191. In some embodiments, an anti-PD-1 antibody, or an antigen-binding fragment thereof, comprises a variable heavy chain comprising the amino acid sequence of SEQ ID NO: 192. In some embodiments, an anti-PD-1 antibody, or an antigen-binding fragment thereof, comprises a variable heavy chain comprising the amino acid sequence of SEQ ID NO: 193. In some embodiments, an anti-PD-1 antibody, or an antigen-binding fragment thereof, comprises a variable heavy chain comprising the amino acid sequence of SEQ ID NO: 194. In some embodiments, an anti-PD-1 antibody, or an antigen-binding fragment thereof, comprises a variable heavy chain comprising the amino acid sequence of SEQ ID NO: 195. In some embodiments, an anti-PD-1 antibody, or an antigen-binding fragment thereof, comprises a variable heavy chain comprising the amino acid sequence of SEQ ID NO: 196. In some embodiments, an anti-PD-1 antibody, or an antigen-binding fragment thereof, comprises a variable heavy chain comprising the amino acid sequence of SEQ ID NO: 197. In some embodiments, an anti-PD-1 antibody, or an antigen-binding fragment thereof, comprises a variable heavy chain comprising the amino acid sequence of SEQ ID NO: 198. In some embodiments, an anti-PD-1 antibody, or an antigen-binding fragment thereof, comprises a variable heavy chain comprising the amino acid sequence of SEQ ID NO: 199. In some embodiments, an anti-PD-1 antibody, or an antigen-binding fragment thereof, comprises a variable heavy chain comprising the amino acid sequence of SEQ ID NO: 200. In some embodiments, an anti-PD-1 antibody, or an antigen-binding fragment thereof, comprises a variable heavy chain comprising the amino acid sequence of SEQ ID NO: 201. In some embodiments, an anti-PD-1 antibody, or an antigen-binding fragment thereof, comprises a variable heavy chain comprising the amino acid sequence of SEQ ID NO: 202. In some embodiments, an anti-PD-1 antibody, or an antigen-binding fragment thereof, comprises a variable heavy chain comprising the amino acid sequence of SEQ ID NO: 203. In some embodiments, an anti-PD-1 antibody, or an antigen-binding fragment thereof, comprises a variable heavy chain comprising the amino acid sequence of SEQ ID NO: 204. In some embodiments, an anti-PD-1 antibody, or an antigen-binding fragment thereof, comprises a variable heavy chain comprising the amino acid sequence of SEQ ID NO: 205. In some embodiments, an anti-PD-1 antibody, or an antigen-binding fragment thereof, comprises a variable heavy chain comprising the amino acid sequence of SEQ ID NO: 206. In some embodiments, an anti-PD-1 antibody, or an antigen-binding fragment thereof, comprises a variable heavy chain comprising the amino acid sequence of SEQ ID NO: 207. In some embodiments, an anti-PD-1 antibody, or an antigen-binding fragment thereof, comprises a variable heavy chain comprising the amino acid sequence of SEQ ID NO: 208. In some embodiments, an anti-PD-1 antibody, or an antigen-binding fragment thereof, comprises a variable heavy chain comprising the amino acid sequence of SEQ ID NO: 209. In some embodiments, an anti-PD-1 antibody, or an antigen-binding fragment thereof, comprises a variable heavy chain comprising the amino acid sequence of SEQ ID NO: 210. In some embodiments, an anti-PD-1 antibody, or an antigen-binding fragment thereof, comprises a variable heavy chain comprising the amino acid sequence of SEQ ID NO: 211. In some embodiments, an anti-PD-1 antibody, or an antigen-binding fragment thereof, comprises a variable heavy chain comprising the amino acid sequence of SEQ ID NO: 212. In some embodiments, an anti-PD-1 antibody, or an antigen-binding fragment thereof, comprises a variable heavy chain comprising the amino acid sequence of SEQ ID NO: 213. In some embodiments, an anti-PD-1 antibody, or an antigen-binding fragment thereof, comprises a variable heavy chain comprising the amino acid sequence of SEQ ID NO: 214. In some embodiments, an anti-PD-1 antibody, or an antigen-binding fragment thereof, comprises a variable heavy chain comprising the amino acid sequence of SEQ ID NO: 215. In some embodiments, an anti-PD-1 antibody, or an antigen-binding fragment thereof, comprises a variable heavy chain comprising the amino acid sequence of SEQ ID NO: 216. In some embodiments, an anti-PD-1 antibody, or an antigen-binding fragment thereof, comprises a variable heavy chain comprising the amino acid sequence of SEQ ID NO: 217. In some embodiments, an anti-PD-1 antibody, or an antigen-binding fragment thereof, comprises a variable heavy chain comprising the amino acid sequence of SEQ ID NO: 218. In some embodiments, an anti-PD-1 antibody, or an antigen-binding fragment thereof, comprises a variable heavy chain comprising the amino acid sequence of SEQ ID NO: 219. In some embodiments, an anti-PD-1 antibody, or an antigen-binding fragment thereof, comprises a variable heavy chain comprising the amino acid sequence of SEQ ID NO: 220. In some embodiments, an anti-PD-1 antibody, or an antigen-binding fragment thereof, comprises a variable heavy chain comprising the amino acid sequence of SEQ ID NO: 221. In some embodiments, an anti-PD-1 antibody, or an antigen-binding fragment thereof, comprises a variable heavy chain comprising the amino acid sequence of SEQ ID NO: 222. In some embodiments, an anti-PD-1 antibody, or an antigen-binding fragment thereof, comprises a variable heavy chain comprising the amino acid sequence of SEQ ID NO: 223. In some embodiments, an anti-PD-1 antibody, or an antigen-binding fragment thereof, comprises a variable heavy chain comprising the amino acid sequence of SEQ ID NO: 224. In some embodiments, an anti-PD-1 antibody, or an antigen-binding fragment thereof, comprises a variable heavy chain comprising the amino acid sequence of SEQ ID NO: 225. In some embodiments, an anti-PD-1 antibody, or an antigen-binding fragment thereof, comprises a variable heavy chain comprising the amino acid sequence of SEQ ID NO: 226. In some embodiments, an anti-PD-1 antibody, or an antigen-binding fragment thereof, comprises a variable heavy chain comprising the amino acid sequence of SEQ ID NO: 227. In some embodiments, an anti-PD-1 antibody, or an antigen-binding fragment thereof, comprises a variable heavy chain comprising the amino acid sequence of SEQ ID NO: 228. In some embodiments, an anti-PD-1 antibody, or an antigen-binding fragment thereof, comprises a variable heavy chain comprising the amino acid sequence of SEQ ID NO: 229. In some embodiments, an anti-PD-1 antibody, or an antigen-binding fragment thereof, comprises a variable heavy chain comprising the amino acid sequence of SEQ ID NO: 230. In some embodiments, an anti-PD-1 antibody, or an antigen-binding fragment thereof, comprises a variable heavy chain comprising the amino acid sequence of SEQ ID NO: 231. In some embodiments, an anti-PD-1 antibody, or an antigen-binding fragment thereof, comprises a variable heavy chain comprising the amino acid sequence of SEQ ID NO: 232. In some embodiments, an anti-PD-1 antibody, or an antigen-binding fragment thereof, comprises a variable heavy chain comprising the amino acid sequence of SEQ ID NO: 233. In some embodiments, an anti-PD-1 antibody, or an antigen-binding fragment thereof, comprises a variable heavy chain comprising the amino acid sequence of SEQ ID NO: 234. In some embodiments, an anti-PD-1 antibody, or an antigen-binding fragment thereof, comprises a variable heavy chain comprising the amino acid sequence of SEQ ID NO: 235. In some embodiments, an anti-PD-1 antibody, or an antigen-binding fragment thereof, comprises a variable heavy chain comprising the amino acid sequence of SEQ ID NO: 236. In some embodiments, an anti-PD-1 antibody, or an antigen-binding fragment thereof, comprises a variable heavy chain comprising the amino acid sequence of SEQ ID NO: 237. In some embodiments, an anti-PD-1 antibody, or an antigen-binding fragment thereof, comprises a variable heavy chain comprising the amino acid sequence of SEQ ID NO: 238. In some embodiments, an anti-PD-1 antibody, or an antigen-binding fragment thereof, comprises a variable heavy chain comprising the amino acid sequence of SEQ ID NO: 239. In some embodiments, an anti-PD-1 antibody, or an antigen-binding fragment thereof, comprises a variable heavy chain comprising the amino acid sequence of SEQ ID NO: 240. In some embodiments, an anti-PD-1 antibody, or an antigen-binding fragment thereof, comprises a variable heavy chain comprising the amino acid sequence of SEQ ID NO: 241. In some embodiments, an anti-PD-1 antibody, or an antigen-binding fragment thereof, comprises a variable heavy chain comprising the amino acid sequence of SEQ ID NO: 242. In some embodiments, an anti-PD-1 antibody, or an antigen-binding fragment thereof, comprises a variable heavy chain comprising the amino acid sequence of SEQ ID NO: 243. In some embodiments, an anti-PD-1 antibody, or an antigen-binding fragment thereof, comprises a variable heavy chain comprising the amino acid sequence of SEQ ID NO: 244. In some embodiments, an anti-PD-1 antibody, or an antigen-binding fragment thereof, comprises a variable heavy chain comprising the amino acid sequence of SEQ ID NO: 245. In some embodiments, an anti-PD-1 antibody, or an antigen-binding fragment thereof, comprises a variable heavy chain comprising the amino acid sequence of SEQ ID NO: 246. In some embodiments, an anti-PD-1 antibody, or an antigen-binding fragment thereof, comprises a variable heavy chain comprising the amino acid sequence of SEQ ID NO: 247. In some embodiments, an anti-PD-1 antibody, or an antigen-binding fragment thereof, comprises a variable heavy chain comprising the amino acid sequence of SEQ ID NO: 248. In some embodiments, an anti-PD-1 antibody, or an antigen-binding fragment thereof, comprises a variable heavy chain comprising the amino acid sequence of SEQ ID NO: 249. In some embodiments, an anti-PD-1 antibody, or an antigen-binding fragment thereof, comprises a variable heavy chain comprising the amino acid sequence of SEQ ID NO: 250. In some embodiments, an anti-PD-1 antibody, or an antigen-binding fragment thereof, comprises a variable heavy chain comprising the amino acid sequence of SEQ ID NO: 251. In some embodiments, an anti-PD-1 antibody, or an antigen-binding fragment thereof, comprises a variable heavy chain comprising the amino acid sequence of SEQ ID NO: 252. In some embodiments, an anti-PD-1 antibody, or an antigen-binding fragment thereof, comprises a variable heavy chain comprising the amino acid sequence of SEQ ID NO: 253. In some embodiments, an anti-PD-1 antibody, or an antigen-binding fragment thereof, comprises a variable heavy chain comprising the amino acid sequence of SEQ ID NO: 254. In some embodiments, an anti-PD-1 antibody, or an antigen-binding fragment thereof, comprises a variable heavy chain comprising the amino acid sequence of SEQ ID NO: 255. In some embodiments, an anti-PD-1 antibody, or an antigen-binding fragment thereof, comprises a variable heavy chain comprising the amino acid sequence of SEQ ID NO: 256. In some embodiments, an anti-PD-1 antibody, or an antigen-binding fragment thereof, comprises a variable heavy chain comprising the amino acid sequence of SEQ ID NO: 257. In some embodiments, an anti-PD-1 antibody, or an antigen-binding fragment thereof, comprises a variable heavy chain comprising the amino acid sequence of SEQ ID NO: 258. In some embodiments, an anti-PD-1 antibody, or an antigen-binding fragment thereof, comprises a variable heavy chain comprising the amino acid sequence of SEQ ID NO: 259. In some embodiments, an anti-PD-1 antibody, or an antigen-binding fragment thereof, comprises a variable heavy chain comprising the amino acid sequence of SEQ ID NO: 260. In some embodiments, an anti-PD-1 antibody, or an antigen-binding fragment thereof, comprises a variable heavy chain comprising the amino acid sequence of SEQ ID NO: 261. In some embodiments, an anti-PD-1 antibody, or an antigen-binding fragment thereof, comprises a variable heavy chain comprising the amino acid sequence of SEQ ID NO: 262. In some embodiments, an anti-PD-1 antibody, or an antigen-binding fragment thereof, comprises a variable heavy chain comprising the amino acid sequence of SEQ ID NO: 263. In some embodiments, an anti-PD-1 antibody, or an antigen-binding fragment thereof, comprises a variable heavy chain comprising the amino acid sequence of SEQ ID NO: 264. In some embodiments, an anti-PD-1 antibody, or an antigen-binding fragment thereof, comprises a variable heavy chain comprising the amino acid sequence of SEQ ID NO: 265. In some embodiments, an anti-PD-1 antibody, or an antigen-binding fragment thereof, comprises a variable heavy chain comprising the amino acid sequence of SEQ ID NO: 266. In some embodiments, an anti-PD-1 antibody, or an antigen-binding fragment thereof, comprises a variable heavy chain comprising the amino acid sequence of SEQ ID NO: 267. In some embodiments, an anti-PD-1 antibody, or an antigen-binding fragment thereof, comprises a variable heavy chain comprising the amino acid sequence of SEQ ID NO: 268. In some embodiments, an anti-PD-1 antibody, or an antigen-binding fragment thereof, comprises a variable heavy chain comprising the amino acid sequence of SEQ ID NO: 269. In some embodiments, an anti-PD-1 antibody, or an antigen-binding fragment thereof, comprises a variable heavy chain comprising the amino acid sequence of SEQ ID NO: 270. In some embodiments, an anti-PD-1 antibody, or an antigen-binding fragment thereof, comprises a variable heavy chain comprising the amino acid sequence of SEQ ID NO: 271. In some embodiments, an anti-PD-1 antibody, or an antigen-binding fragment thereof, comprises a variable heavy chain comprising the amino acid sequence of SEQ ID NO: 272. In some embodiments, an anti-PD-1 antibody, or an antigen-binding fragment thereof, comprises a variable heavy chain comprising the amino acid sequence of SEQ ID NO: 273. In some embodiments, an anti-PD-1 antibody, or an antigen-binding fragment thereof, comprises a variable heavy chain comprising the amino acid sequence of SEQ ID NO: 274. In some embodiments, an anti-PD-1 antibody, or an antigen-binding fragment thereof, comprises a variable heavy chain comprising the amino acid sequence of SEQ ID NO: 275. In some embodiments, an anti-PD-1 antibody, or an antigen-binding fragment thereof, comprises a variable heavy chain comprising the amino acid sequence of SEQ ID NO: 276. In some embodiments, an anti-PD-1 antibody, or an antigen-binding fragment thereof, comprises a variable heavy chain comprising the amino acid sequence of SEQ ID NO: 277. In some embodiments, an anti-PD-1 antibody, or an antigen-binding fragment thereof, comprises a variable heavy chain comprising the amino acid sequence of SEQ ID NO: 278. In some embodiments, an anti-PD-1 antibody, or an antigen-binding fragment thereof, comprises a variable heavy chain comprising the amino acid sequence of SEQ ID NO: 279. In some embodiments, an anti-PD-1 antibody, or an antigen-binding fragment thereof, comprises a variable heavy chain comprising the amino acid sequence of SEQ ID NO: 280. In some embodiments, an anti-PD-1 antibody, or an antigen-binding fragment thereof, comprises a variable heavy chain comprising the amino acid sequence of SEQ ID NO: 281. In some embodiments, an anti-PD-1 antibody, or an antigen-binding fragment thereof, comprises a variable heavy chain comprising the amino acid sequence of SEQ ID NO: 282. In some embodiments, an anti-PD-1 antibody, or an antigen-binding fragment thereof, comprises a variable heavy chain comprising the amino acid sequence of SEQ ID NO: 283. In some embodiments, an anti-PD-1 antibody, or an antigen-binding fragment thereof, comprises a variable heavy chain comprising the amino acid sequence of SEQ ID NO: 284. In some embodiments, an anti-PD-1 antibody, or an antigen-binding fragment thereof, comprises a variable heavy chain comprising the amino acid sequence of SEQ ID NO: 285. In some embodiments, an anti-PD-1 antibody, or an antigen-binding fragment thereof, comprises a variable heavy chain comprising the amino acid sequence of SEQ ID NO: 286. In some embodiments, an anti-PD-1 antibody, or an antigen-binding fragment thereof, comprises a variable heavy chain comprising the amino acid sequence of SEQ ID NO: 287. In some embodiments, an anti-PD-1 antibody, or an antigen-binding fragment thereof, comprises a variable heavy chain comprising the amino acid sequence of SEQ ID NO: 288. In some embodiments, an anti-PD-1 antibody, or an antigen-binding fragment thereof, comprises a variable heavy chain comprising the amino acid sequence of SEQ ID NO: 289. In some embodiments, an anti-PD-1 antibody, or an antigen-binding fragment thereof, comprises a variable heavy chain comprising the amino acid sequence of SEQ ID NO: 290. In some embodiments, an anti-PD-1 antibody, or an antigen-binding fragment thereof, comprises a variable heavy chain comprising the amino acid sequence of SEQ ID NO: 291. In some embodiments, an anti-PD-1 antibody, or an antigen-binding fragment thereof, comprises a variable heavy chain comprising the amino acid sequence of SEQ ID NO: 292. In some embodiments, an anti-PD-1 antibody, or an antigen-binding fragment thereof, comprises a variable heavy chain comprising the amino acid sequence of SEQ ID NO: 293. In some embodiments, an anti-PD-1 antibody, or an antigen-binding fragment thereof, comprises a variable heavy chain comprising the amino acid sequence of SEQ ID NO: 294. In some embodiments, an anti-PD-1 antibody, or an antigen-binding fragment thereof, comprises a variable heavy chain comprising the amino acid sequence of SEQ ID NO: 295. In some embodiments, an anti-PD-1 antibody, or an antigen-binding fragment thereof, comprises a variable heavy chain comprising the amino acid sequence of SEQ ID NO: 296. In some embodiments, an anti-PD-1 antibody, or an antigen-binding fragment thereof, comprises a variable heavy chain comprising the amino acid sequence of SEQ ID NO: 297. In some embodiments, an anti-PD-1 antibody, or an antigen-binding fragment thereof, comprises a variable heavy chain comprising the amino acid sequence of SEQ ID NO: 298. In some embodiments, an anti-PD-1 antibody, or an antigen-binding fragment thereof, comprises a variable heavy chain comprising the amino acid sequence of SEQ ID NO: 299. In some embodiments, an anti-PD-1 antibody, or an antigen-binding fragment thereof, comprises a variable heavy chain comprising the amino acid sequence of SEQ ID NO: 300. In some embodiments, an anti-PD-1 antibody, or an antigen-binding fragment thereof, comprises a variable heavy chain comprising the amino acid sequence of SEQ ID NO: 301. In some embodiments, an anti-PD-1 antibody, or an antigen-binding fragment thereof, comprises a variable heavy chain comprising the amino acid sequence of SEQ ID NO: 302. In some embodiments, an anti-PD-1 antibody, or an antigen-binding fragment thereof, comprises a variable heavy chain comprising the amino acid sequence of SEQ ID NO: 303. In some embodiments, an anti-PD-1 antibody, or an antigen-binding fragment thereof, comprises a variable heavy chain comprising the amino acid sequence of SEQ ID NO: 304. In some embodiments, an anti-PD-1 antibody, or an antigen-binding fragment thereof, comprises a variable heavy chain comprising the amino acid sequence of SEQ ID NO: 305. In some embodiments, an anti-PD-1 antibody, or an antigen-binding fragment thereof, comprises a variable heavy chain comprising the amino acid sequence of SEQ ID NO: 306. In some embodiments, an anti-PD-1 antibody, or an antigen-binding fragment thereof, comprises a variable heavy chain comprising the amino acid sequence of SEQ ID NO: 307. In some embodiments, an anti-PD-1 antibody, or an antigen-binding fragment thereof, comprises a variable heavy chain comprising the amino acid sequence of SEQ ID NO: 308. In some embodiments, an anti-PD-1 antibody, or an antigen-binding fragment thereof, comprises a variable heavy chain comprising the amino acid sequence of SEQ ID NO: 309. In some embodiments, an anti-PD-1 antibody, or an antigen-binding fragment thereof, comprises a variable heavy chain comprising the amino acid sequence of SEQ ID NO: 310. In some embodiments, an anti-PD-1 antibody, or an antigen-binding fragment thereof, comprises a variable heavy chain comprising the amino acid sequence of SEQ ID NO: 311. In some embodiments, an anti-PD-1 antibody, or an antigen-binding fragment thereof, comprises a variable heavy chain comprising the amino acid sequence of SEQ ID NO: 312. In some embodiments, an anti-PD-1 antibody, or an antigen-binding fragment thereof, comprises a variable heavy chain comprising the amino acid sequence of SEQ ID NO: 313. In some embodiments, an anti-PD-1 antibody, or an antigen-binding fragment thereof, comprises a variable heavy chain comprising the amino acid sequence of SEQ ID NO: 314. In some embodiments, an anti-PD-1 antibody, or an antigen-binding fragment thereof, comprises a variable heavy chain comprising the amino acid sequence of SEQ ID NO: 315. In some embodiments, an anti-PD-1 antibody, or an antigen-binding fragment thereof, comprises a variable heavy chain comprising the amino acid sequence of SEQ ID NO: 316. In some embodiments, an anti-PD-1 antibody, or an antigen-binding fragment thereof, comprises a variable heavy chain comprising the amino acid sequence of SEQ ID NO: 317. In some embodiments, an anti-PD-1 antibody, or an antigen-binding fragment thereof, comprises a variable heavy chain comprising the amino acid sequence of SEQ ID NO: 318. In some embodiments, an anti-PD-1 antibody, or an antigen-binding fragment thereof, comprises a variable heavy chain comprising the amino acid sequence of SEQ ID NO: 319. In some embodiments, an anti-PD-1 antibody, or an antigen-binding fragment thereof, comprises a variable heavy chain comprising the amino acid sequence of SEQ ID NO: 320. In some embodiments, an anti-PD-1 antibody, or an antigen-binding fragment thereof, comprises a variable heavy chain comprising the amino acid sequence of SEQ ID NO: 321. In some embodiments, an anti-PD-1 antibody, or an antigen-binding fragment thereof, comprises a variable heavy chain comprising the amino acid sequence of SEQ ID NO: 322. In some embodiments, an anti-PD-1 antibody, or an antigen-binding fragment thereof, comprises a variable heavy chain comprising the amino acid sequence of SEQ ID NO: 530. In some embodiments, an anti-PD-1 antibody, or an antigen-binding fragment thereof, comprises a variable heavy chain comprising the amino acid sequence of SEQ ID NO: 538. In some embodiments, an anti-PD-1 antibody, or an antigen-binding fragment thereof, comprises a variable heavy chain comprising the amino acid sequence of SEQ ID NO: 540.

In some embodiments, an anti-PD-1 antibody, or an antigen-binding fragment thereof, comprises a variable light chain comprising an amino acid sequence selected from any one of SEQ ID NOs: 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 531, 532, 533, 534, 535, 536, 537, 539, 541, and 542.

In some embodiments, an anti-PD-1 antibody, or an antigen-binding fragment thereof, comprises a variable light chain comprising the amino acid sequence of SEQ ID NO: 323. In some embodiments, an anti-PD-1 antibody, or an antigen-binding fragment thereof, comprises a variable light chain comprising the amino acid sequence of SEQ ID NO: 324. In some embodiments, an anti-PD-1 antibody, or an antigen-binding fragment thereof, comprises a variable light chain comprising the amino acid sequence of SEQ ID NO: 325. In some embodiments, an anti-PD-1 antibody, or an antigen-binding fragment thereof, comprises a variable light chain comprising the amino acid sequence of SEQ ID NO: 326. In some embodiments, an anti-PD-1 antibody, or an antigen-binding fragment thereof, comprises a variable light chain comprising the amino acid sequence of SEQ ID NO: 327. In some embodiments, an anti-PD-1 antibody, or an antigen-binding fragment thereof, comprises a variable light chain comprising the amino acid sequence of SEQ ID NO: 328. In some embodiments, an anti-PD-1 antibody, or an antigen-binding fragment thereof, comprises a variable light chain comprising the amino acid sequence of SEQ ID NO: 329. In some embodiments, an anti-PD-1 antibody, or an antigen-binding fragment thereof, comprises a variable light chain comprising the amino acid sequence of SEQ ID NO: 330. In some embodiments, an anti-PD-1 antibody, or an antigen-binding fragment thereof, comprises a variable light chain comprising the amino acid sequence of SEQ ID NO: 331. In some embodiments, an anti-PD-1 antibody, or an antigen-binding fragment thereof, comprises a variable light chain comprising the amino acid sequence of SEQ ID NO: 332. In some embodiments, an anti-PD-1 antibody, or an antigen-binding fragment thereof, comprises a variable light chain comprising the amino acid sequence of SEQ ID NO: 333. In some embodiments, an anti-PD-1 antibody, or an antigen-binding fragment thereof, comprises a variable light chain comprising the amino acid sequence of SEQ ID NO: 334. In some embodiments, an anti-PD-1 antibody, or an antigen-binding fragment thereof, comprises a variable light chain comprising the amino acid sequence of SEQ ID NO: 335. In some embodiments, an anti-PD-1 antibody, or an antigen-binding fragment thereof, comprises a variable light chain comprising the amino acid sequence of SEQ ID NO: 336. In some embodiments, an anti-PD-1 antibody, or an antigen-binding fragment thereof, comprises a variable light chain comprising the amino acid sequence of SEQ ID NO: 337. In some embodiments, an anti-PD-1 antibody, or an antigen-binding fragment thereof, comprises a variable light chain comprising the amino acid sequence of SEQ ID NO: 338. In some embodiments, an anti-PD-1 antibody, or an antigen-binding fragment thereof, comprises a variable light chain comprising the amino acid sequence of SEQ ID NO: 339. In some embodiments, an anti-PD-1 antibody, or an antigen-binding fragment thereof, comprises a variable light chain comprising the amino acid sequence of SEQ ID NO: 340. In some embodiments, an anti-PD-1 antibody, or an antigen-binding fragment thereof, comprises a variable light chain comprising the amino acid sequence of SEQ ID NO: 341. In some embodiments, an anti-PD-1 antibody, or an antigen-binding fragment thereof, comprises a variable light chain comprising the amino acid sequence of SEQ ID NO: 342. In some embodiments, an anti-PD-1 antibody, or an antigen-binding fragment thereof, comprises a variable light chain comprising the amino acid sequence of SEQ ID NO: 343. In some embodiments, an anti-PD-1 antibody, or an antigen-binding fragment thereof, comprises a variable light chain comprising the amino acid sequence of SEQ ID NO: 344. In some embodiments, an anti-PD-1 antibody, or an antigen-binding fragment thereof, comprises a variable light chain comprising the amino acid sequence of SEQ ID NO: 345. In some embodiments, an anti-PD-1 antibody, or an antigen-binding fragment thereof, comprises a variable light chain comprising the amino acid sequence of SEQ ID NO: 346. In some embodiments, an anti-PD-1 antibody, or an antigen-binding fragment thereof, comprises a variable light chain comprising the amino acid sequence of SEQ ID NO: 347. In some embodiments, an anti-PD-1 antibody, or an antigen-binding fragment thereof, comprises a variable light chain comprising the amino acid sequence of SEQ ID NO: 348. In some embodiments, an anti-PD-1 antibody, or an antigen-binding fragment thereof, comprises a variable light chain comprising the amino acid sequence of SEQ ID NO: 349. In some embodiments, an anti-PD-1 antibody, or an antigen-binding fragment thereof, comprises a variable light chain comprising the amino acid sequence of SEQ ID NO: 350. In some embodiments, an anti-PD-1 antibody, or an antigen-binding fragment thereof, comprises a variable light chain comprising the amino acid sequence of SEQ ID NO: 351. In some embodiments, an anti-PD-1 antibody, or an antigen-binding fragment thereof, comprises a variable light chain comprising the amino acid sequence of SEQ ID NO: 352. In some embodiments, an anti-PD-1 antibody, or an antigen-binding fragment thereof, comprises a variable light chain comprising the amino acid sequence of SEQ ID NO: 353. In some embodiments, an anti-PD-1 antibody, or an antigen-binding fragment thereof, comprises a variable light chain comprising the amino acid sequence of SEQ ID NO: 354. In some embodiments, an anti-PD-1 antibody, or an antigen-binding fragment thereof, comprises a variable light chain comprising the amino acid sequence of SEQ ID NO: 355. In some embodiments, an anti-PD-1 antibody, or an antigen-binding fragment thereof, comprises a variable light chain comprising the amino acid sequence of SEQ ID NO: 356. In some embodiments, an anti-PD-1 antibody, or an antigen-binding fragment thereof, comprises a variable light chain comprising the amino acid sequence of SEQ ID NO: 357. In some embodiments, an anti-PD-1 antibody, or an antigen-binding fragment thereof, comprises a variable light chain comprising the amino acid sequence of SEQ ID NO: 358. In some embodiments, an anti-PD-1 antibody, or an antigen-binding fragment thereof, comprises a variable light chain comprising the amino acid sequence of SEQ ID NO: 359. In some embodiments, an anti-PD-1 antibody, or an antigen-binding fragment thereof, comprises a variable light chain comprising the amino acid sequence of SEQ ID NO: 360. In some embodiments, an anti-PD-1 antibody, or an antigen-binding fragment thereof, comprises a variable light chain comprising the amino acid sequence of SEQ ID NO: 361. In some embodiments, an anti-PD-1 antibody, or an antigen-binding fragment thereof, comprises a variable light chain comprising the amino acid sequence of SEQ ID NO: 362. In some embodiments, an anti-PD-1 antibody, or an antigen-binding fragment thereof, comprises a variable light chain comprising the amino acid sequence of SEQ ID NO: 363. In some embodiments, an anti-PD-1 antibody, or an antigen-binding fragment thereof, comprises a variable light chain comprising the amino acid sequence of SEQ ID NO: 364. In some embodiments, an anti-PD-1 antibody, or an antigen-binding fragment thereof, comprises a variable light chain comprising the amino acid sequence of SEQ ID NO: 365. In some embodiments, an anti-PD-1 antibody, or an antigen-binding fragment thereof, comprises a variable light chain comprising the amino acid sequence of SEQ ID NO: 366. In some embodiments, an anti-PD-1 antibody, or an antigen-binding fragment thereof, comprises a variable light chain comprising the amino acid sequence of SEQ ID NO: 367. In some embodiments, an anti-PD-1 antibody, or an antigen-binding fragment thereof, comprises a variable light chain comprising the amino acid sequence of SEQ ID NO: 368. In some embodiments, an anti-PD-1 antibody, or an antigen-binding fragment thereof, comprises a variable light chain comprising the amino acid sequence of SEQ ID NO: 369. In some embodiments, an anti-PD-1 antibody, or an antigen-binding fragment thereof, comprises a variable light chain comprising the amino acid sequence of SEQ ID NO: 370. In some embodiments, an anti-PD-1 antibody, or an antigen-binding fragment thereof, comprises a variable light chain comprising the amino acid sequence of SEQ ID NO: 371. In some embodiments, an anti-PD-1 antibody, or an antigen-binding fragment thereof, comprises a variable light chain comprising the amino acid sequence of SEQ ID NO: 372. In some embodiments, an anti-PD-1 antibody, or an antigen-binding fragment thereof, comprises a variable light chain comprising the amino acid sequence of SEQ ID NO: 373. In some embodiments, an anti-PD-1 antibody, or an antigen-binding fragment thereof, comprises a variable light chain comprising the amino acid sequence of SEQ ID NO: 374. In some embodiments, an anti-PD-1 antibody, or an antigen-binding fragment thereof, comprises a variable light chain comprising the amino acid sequence of SEQ ID NO: 375. In some embodiments, an anti-PD-1 antibody, or an antigen-binding fragment thereof, comprises a variable light chain comprising the amino acid sequence of SEQ ID NO: 376. In some embodiments, an anti-PD-1 antibody, or an antigen-binding fragment thereof, comprises a variable light chain comprising the amino acid sequence of SEQ ID NO: 377. In some embodiments, an anti-PD-1 antibody, or an antigen-binding fragment thereof, comprises a variable light chain comprising the amino acid sequence of SEQ ID NO: 378. In some embodiments, an anti-PD-1 antibody, or an antigen-binding fragment thereof, comprises a variable light chain comprising the amino acid sequence of SEQ ID NO: 379. In some embodiments, an anti-PD-1 antibody, or an antigen-binding fragment thereof, comprises a variable light chain comprising the amino acid sequence of SEQ ID NO: 380. In some embodiments, an anti-PD-1 antibody, or an antigen-binding fragment thereof, comprises a variable light chain comprising the amino acid sequence of SEQ ID NO: 381. In some embodiments, an anti-PD-1 antibody, or an antigen-binding fragment thereof, comprises a variable light chain comprising the amino acid sequence of SEQ ID NO: 382. In some embodiments, an anti-PD-1 antibody, or an antigen-binding fragment thereof, comprises a variable light chain comprising the amino acid sequence of SEQ ID NO: 383. In some embodiments, an anti-PD-1 antibody, or an antigen-binding fragment thereof, comprises a variable light chain comprising the amino acid sequence of SEQ ID NO: 384. In some embodiments, an anti-PD-1 antibody, or an antigen-binding fragment thereof, comprises a variable light chain comprising the amino acid sequence of SEQ ID NO: 385. In some embodiments, an anti-PD-1 antibody, or an antigen-binding fragment thereof, comprises a variable light chain comprising the amino acid sequence of SEQ ID NO: 386. In some embodiments, an anti-PD-1 antibody, or an antigen-binding fragment thereof, comprises a variable light chain comprising the amino acid sequence of SEQ ID NO: 387. In some embodiments, an anti-PD-1 antibody, or an antigen-binding fragment thereof, comprises a variable light chain comprising the amino acid sequence of SEQ ID NO: 388. In some embodiments, an anti-PD-1 antibody, or an antigen-binding fragment thereof, comprises a variable light chain comprising the amino acid sequence of SEQ ID NO: 389. In some embodiments, an anti-PD-1 antibody, or an antigen-binding fragment thereof, comprises a variable light chain comprising the amino acid sequence of SEQ ID NO: 390. In some embodiments, an anti-PD-1 antibody, or an antigen-binding fragment thereof, comprises a variable light chain comprising the amino acid sequence of SEQ ID NO: 391. In some embodiments, an anti-PD-1 antibody, or an antigen-binding fragment thereof, comprises a variable light chain comprising the amino acid sequence of SEQ ID NO: 392. In some embodiments, an anti-PD-1 antibody, or an antigen-binding fragment thereof, comprises a variable light chain comprising the amino acid sequence of SEQ ID NO: 393. In some embodiments, an anti-PD-1 antibody, or an antigen-binding fragment thereof, comprises a variable light chain comprising the amino acid sequence of SEQ ID NO: 394. In some embodiments, an anti-PD-1 antibody, or an antigen-binding fragment thereof, comprises a variable light chain comprising the amino acid sequence of SEQ ID NO: 395. In some embodiments, an anti-PD-1 antibody, or an antigen-binding fragment thereof, comprises a variable light chain comprising the amino acid sequence of SEQ ID NO: 396. In some embodiments, an anti-PD-1 antibody, or an antigen-binding fragment thereof, comprises a variable light chain comprising the amino acid sequence of SEQ ID NO: 397. In some embodiments, an anti-PD-1 antibody, or an antigen-binding fragment thereof, comprises a variable light chain comprising the amino acid sequence of SEQ ID NO: 398. In some embodiments, an anti-PD-1 antibody, or an antigen-binding fragment thereof, comprises a variable light chain comprising the amino acid sequence of SEQ ID NO: 399. In some embodiments, an anti-PD-1 antibody, or an antigen-binding fragment thereof, comprises a variable light chain comprising the amino acid sequence of SEQ ID NO: 400. In some embodiments, an anti-PD-1 antibody, or an antigen-binding fragment thereof, comprises a variable light chain comprising the amino acid sequence of SEQ ID NO: 401. In some embodiments, an anti-PD-1 antibody, or an antigen-binding fragment thereof, comprises a variable light chain comprising the amino acid sequence of SEQ ID NO: 402. In some embodiments, an anti-PD-1 antibody, or an antigen-binding fragment thereof, comprises a variable light chain comprising the amino acid sequence of SEQ ID NO: 403. In some embodiments, an anti-PD-1 antibody, or an antigen-binding fragment thereof, comprises a variable light chain comprising the amino acid sequence of SEQ ID NO: 404. In some embodiments, an anti-PD-1 antibody, or an antigen-binding fragment thereof, comprises a variable light chain comprising the amino acid sequence of SEQ ID NO: 405. In some embodiments, an anti-PD-1 antibody, or an antigen-binding fragment thereof, comprises a variable light chain comprising the amino acid sequence of SEQ ID NO: 406. In some embodiments, an anti-PD-1 antibody, or an antigen-binding fragment thereof, comprises a variable light chain comprising the amino acid sequence of SEQ ID NO: 407. In some embodiments, an anti-PD-1 antibody, or an antigen-binding fragment thereof, comprises a variable light chain comprising the amino acid sequence of SEQ ID NO: 408. In some embodiments, an anti-PD-1 antibody, or an antigen-binding fragment thereof, comprises a variable light chain comprising the amino acid sequence of SEQ ID NO: 409. In some embodiments, an anti-PD-1 antibody, or an antigen-binding fragment thereof, comprises a variable light chain comprising the amino acid sequence of SEQ ID NO: 410. In some embodiments, an anti-PD-1 antibody, or an antigen-binding fragment thereof, comprises a variable light chain comprising the amino acid sequence of SEQ ID NO: 411. In some embodiments, an anti-PD-1 antibody, or an antigen-binding fragment thereof, comprises a variable light chain comprising the amino acid sequence of SEQ ID NO: 412. In some embodiments, an anti-PD-1 antibody, or an antigen-binding fragment thereof, comprises a variable light chain comprising the amino acid sequence of SEQ ID NO: 413. In some embodiments, an anti-PD-1 antibody, or an antigen-binding fragment thereof, comprises a variable light chain comprising the amino acid sequence of SEQ ID NO: 414. In some embodiments, an anti-PD-1 antibody, or an antigen-binding fragment thereof, comprises a variable light chain comprising the amino acid sequence of SEQ ID NO: 531. In some embodiments, an anti-PD-1 antibody, or an antigen-binding fragment thereof, comprises a variable light chain comprising the amino acid sequence of SEQ ID NO: 532. In some embodiments, an anti-PD-1 antibody, or an antigen-binding fragment thereof, comprises a variable light chain comprising the amino acid sequence of SEQ ID NO: 533. In some embodiments, an anti-PD-1 antibody, or an antigen-binding fragment thereof, comprises a variable light chain comprising the amino acid sequence of SEQ ID NO: 534. In some embodiments, an anti-PD-1 antibody, or an antigen-binding fragment thereof, comprises a variable light chain comprising the amino acid sequence of SEQ ID NO: 535. In some embodiments, an anti-PD-1 antibody, or an antigen-binding fragment thereof, comprises a variable light chain comprising the amino acid sequence of SEQ ID NO: 536. In some embodiments, an anti-PD-1 antibody, or an antigen-binding fragment thereof, comprises a variable light chain comprising the amino acid sequence of SEQ ID NO: 537. In some embodiments, an anti-PD-1 antibody, or an antigen-binding fragment thereof, comprises a variable light chain comprising the amino acid sequence of SEQ ID NO: 539. In some embodiments, an anti-PD-1 antibody, or an antigen-binding fragment thereof, comprises a variable light chain comprising the amino acid sequence of SEQ ID NO: 541. In some embodiments, an anti-PD-1 antibody, or an antigen-binding fragment thereof, comprises a variable light chain comprising the amino acid sequence of SEQ ID NO: 542.

In some embodiments, an anti-PD-1 antibody, or an antigen-binding fragment thereof, comprises a variable heavy chain comprising an amino acid sequence selected from any one of SEQ ID NOs: 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 530, 538, and 540; and a variable light chain comprising an amino acid sequence selected from any one of SEQ ID NOs: 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 531, 532, 533, 534, 535, 536, 537, 539, 541, and 542.

In some embodiments, an anti-PD-1 antibody, or an antigen-binding fragment thereof, comprises a variable heavy chain comprising the amino acid sequence of SEQ ID NO: 130; and a variable light chain comprising an amino acid sequence of SEQ ID NO: 323 In some embodiments, an anti-PD-1 antibody, or an antigen-binding fragment thereof, comprises a variable heavy chain comprising the amino acid sequence of SEQ ID NO: 131; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 132; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 133; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 134; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 135; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 136; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 137; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 138; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 139; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 140; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 141; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 142; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 143; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 144; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 145; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 146; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 147; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 148; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 149; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 150; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 151; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 152; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 153; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 154; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 155; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 156; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 157; and a variable light chain comprising an amino acid sequence of SEQ ID NO: 324. In some embodiments, an anti-PD-1 antibody, or an antigen-binding fragment thereof, comprises a variable heavy chain comprising the amino acid sequence of SEQ ID NO: 158; and a variable light chain comprising an amino acid sequence of SEQ ID NO: 325. In some embodiments, an anti-PD-1 antibody, or an antigen-binding fragment thereof, comprises a variable heavy chain comprising the amino acid sequence of SEQ ID NO: 159; and a variable light chain comprising an amino acid sequence of SEQ ID NO: 326. In some embodiments, an anti-PD-1 antibody, or an antigen-binding fragment thereof, comprises a variable heavy chain comprising the amino acid sequence of SEQ ID NO: 160; and a variable light chain comprising an amino acid sequence of SEQ ID NO: 327. In some embodiments, an anti-PD-1 antibody, or an antigen-binding fragment thereof, comprises a variable heavy chain comprising the amino acid sequence of SEQ ID NO: 161; and a variable light chain comprising an amino acid sequence of SEQ ID NO: 328. In some embodiments, an anti-PD-1 antibody, or an antigen-binding fragment thereof, comprises a variable heavy chain comprising the amino acid sequence of SEQ ID NO: 162; and a variable light chain comprising an amino acid sequence of SEQ ID NO: 326. In some embodiments, an anti-PD-1 antibody, or an antigen-binding fragment thereof, comprises a variable heavy chain comprising the amino acid sequence of SEQ ID NO: 163; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 164; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 165; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 166; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 167; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 168; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 169; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 170; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 171; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 172; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 173; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 174; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 175; and a variable light chain comprising an amino acid sequence of SEQ ID NO: 329. In some embodiments, an anti-PD-1 antibody, or an antigen-binding fragment thereof, comprises a variable heavy chain comprising the amino acid sequence of SEQ ID NO: 176; and a variable light chain comprising an amino acid sequence of SEQ ID NO: 330. In some embodiments, an anti-PD-1 antibody, or an antigen-binding fragment thereof, comprises a variable heavy chain comprising the amino acid sequence of SEQ ID NO: 177; and a variable light chain comprising an amino acid sequence of SEQ ID NO: 331. In some embodiments, an anti-PD-1 antibody, or an antigen-binding fragment thereof, comprises a variable heavy chain comprising the amino acid sequence of SEQ ID NO: 175; and a variable light chain comprising an amino acid sequence of SEQ ID NO: 332. In some embodiments, an anti-PD-1 antibody, or an antigen-binding fragment thereof, comprises a variable heavy chain comprising the amino acid sequence of SEQ ID NO: 178; and a variable light chain comprising an amino acid sequence of SEQ ID NO: 333. In some embodiments, an anti-PD-1 antibody, or an antigen-binding fragment thereof, comprises a variable heavy chain comprising the amino acid sequence of SEQ ID NO: 179; and a variable light chain comprising an amino acid sequence of SEQ ID NO: 334. In some embodiments, an anti-PD-1 antibody, or an antigen-binding fragment thereof, comprises a variable heavy chain comprising the amino acid sequence of SEQ ID NO: 180; and a variable light chain comprising an amino acid sequence of SEQ ID NO: 335. In some embodiments, an anti-PD-1 antibody, or an antigen-binding fragment thereof, comprises a variable heavy chain comprising the amino acid sequence of SEQ ID NO: 181; and a variable light chain comprising an amino acid sequence of SEQ ID NO: 336. In some embodiments, an anti-PD-1 antibody, or an antigen-binding fragment thereof, comprises a variable heavy chain comprising the amino acid sequence of SEQ ID NO: 182; and a variable light chain comprising an amino acid sequence of SEQ ID NO: 337. In some embodiments, an anti-PD-1 antibody, or an antigen-binding fragment thereof, comprises a variable heavy chain comprising the amino acid sequence of SEQ ID NO: 183; and a variable light chain comprising an amino acid sequence of SEQ ID NO: 338. In some embodiments, an anti-PD-1 antibody, or an antigen-binding fragment thereof, comprises a variable heavy chain comprising the amino acid sequence of SEQ ID NO: 184; and a variable light chain comprising an amino acid sequence of SEQ ID NO: 339. In some embodiments, an anti-PD-1 antibody, or an antigen-binding fragment thereof, comprises a variable heavy chain comprising the amino acid sequence of SEQ ID NO: 185; and a variable light chain comprising an amino acid sequence of SEQ ID NO: 340. In some embodiments, an anti-PD-1 antibody, or an antigen-binding fragment thereof, comprises a variable heavy chain comprising the amino acid sequence of SEQ ID NO: 186; and a variable light chain comprising an amino acid sequence of SEQ ID NO: 341. In some embodiments, an anti-PD-1 antibody, or an antigen-binding fragment thereof, comprises a variable heavy chain comprising the amino acid sequence of SEQ ID NO: 186; and a variable light chain comprising an amino acid sequence of SEQ ID NO: 342. In some embodiments, an anti-PD-1 antibody, or an antigen-binding fragment thereof, comprises a variable heavy chain comprising the amino acid sequence of SEQ ID NO: 180; and a variable light chain comprising an amino acid sequence of SEQ ID NO: 343. In some embodiments, an anti-PD-1 antibody, or an antigen-binding fragment thereof, comprises a variable heavy chain comprising the amino acid sequence of SEQ ID NO: 187; and a variable light chain comprising an amino acid sequence of SEQ ID NO: 344. In some embodiments, an anti-PD-1 antibody, or an antigen-binding fragment thereof, comprises a variable heavy chain comprising the amino acid sequence of SEQ ID NO: 183; and a variable light chain comprising an amino acid sequence of SEQ ID NO: 344. In some embodiments, an anti-PD-1 antibody, or an antigen-binding fragment thereof, comprises a variable heavy chain comprising the amino acid sequence of SEQ ID NO: 185; and a variable light chain comprising an amino acid sequence of SEQ ID NO: 345. In some embodiments, an anti-PD-1 antibody, or an antigen-binding fragment thereof, comprises a variable heavy chain comprising the amino acid sequence of SEQ ID NO: 188; and a variable light chain comprising an amino acid sequence of SEQ ID NO: 346. In some embodiments, an anti-PD-1 antibody, or an antigen-binding fragment thereof, comprises a variable heavy chain comprising the amino acid sequence of SEQ ID NO: 189; and a variable light chain comprising an amino acid sequence of SEQ ID NO: 347. In some embodiments, an anti-PD-1 antibody, or an antigen-binding fragment thereof, comprises a variable heavy chain comprising the amino acid sequence of SEQ ID NO: 182; and a variable light chain comprising an amino acid sequence of SEQ ID NO: 346. In some embodiments, an anti-PD-1 antibody, or an antigen-binding fragment thereof, comprises a variable heavy chain comprising the amino acid sequence of SEQ ID NO: 190; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 191; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 192; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 193; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 194; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 195; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 196; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 156; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 197; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 198; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 199; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 200; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 201; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 202; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 203; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 204; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 205; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 206; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 130; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 207; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 208; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 209; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 135; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 210; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 211; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 212; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 204; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 213; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 214; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 215; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 216; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 217; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 218; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 219; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 220; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 221; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 222; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 223; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 224; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 225; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 226; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 227; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 228; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 158; and a variable light chain comprising an amino acid sequence of SEQ ID NO: 348. In some embodiments, an anti-PD-1 antibody, or an antigen-binding fragment thereof, comprises a variable heavy chain comprising the amino acid sequence of SEQ ID NO: 229; and a variable light chain comprising an amino acid sequence of SEQ ID NO: 349. In some embodiments, an anti-PD-1 antibody, or an antigen-binding fragment thereof, comprises a variable heavy chain comprising the amino acid sequence of SEQ ID NO: 230; and a variable light chain comprising an amino acid sequence of SEQ ID NO: 350. In some embodiments, an anti-PD-1 antibody, or an antigen-binding fragment thereof, comprises a variable heavy chain comprising the amino acid sequence of SEQ ID NO: 160; and a variable light chain comprising an amino acid sequence of SEQ ID NO: 351. In some embodiments, an anti-PD-1 antibody, or an antigen-binding fragment thereof, comprises a variable heavy chain comprising the amino acid sequence of SEQ ID NO: 231; and a variable light chain comprising an amino acid sequence of SEQ ID NO: 352. In some embodiments, an anti-PD-1 antibody, or an antigen-binding fragment thereof, comprises a variable heavy chain comprising the amino acid sequence of SEQ ID NO: 232; and a variable light chain comprising an amino acid sequence of SEQ ID NO: 353. In some embodiments, an anti-PD-1 antibody, or an antigen-binding fragment thereof, comprises a variable heavy chain comprising the amino acid sequence of SEQ ID NO: 233; and a variable light chain comprising an amino acid sequence of SEQ ID NO: 354. In some embodiments, an anti-PD-1 antibody, or an antigen-binding fragment thereof, comprises a variable heavy chain comprising the amino acid sequence of SEQ ID NO: 234; and a variable light chain comprising an amino acid sequence of SEQ ID NO: 355. In some embodiments, an anti-PD-1 antibody, or an antigen-binding fragment thereof, comprises a variable heavy chain comprising the amino acid sequence of SEQ ID NO: 235; and a variable light chain comprising an amino acid sequence of SEQ ID NO: 356. In some embodiments, an anti-PD-1 antibody, or an antigen-binding fragment thereof, comprises a variable heavy chain comprising the amino acid sequence of SEQ ID NO: 236; and a variable light chain comprising an amino acid sequence of SEQ ID NO: 357. In some embodiments, an anti-PD-1 antibody, or an antigen-binding fragment thereof, comprises a variable heavy chain comprising the amino acid sequence of SEQ ID NO: 237; and a variable light chain comprising an amino acid sequence of SEQ ID NO: 358. In some embodiments, an anti-PD-1 antibody, or an antigen-binding fragment thereof, comprises a variable heavy chain comprising the amino acid sequence of SEQ ID NO: 238; and a variable light chain comprising an amino acid sequence of SEQ ID NO: 359. In some embodiments, an anti-PD-1 antibody, or an antigen-binding fragment thereof, comprises a variable heavy chain comprising the amino acid sequence of SEQ ID NO: 239; and a variable light chain comprising an amino acid sequence of SEQ ID NO: 328. In some embodiments, an anti-PD-1 antibody, or an antigen-binding fragment thereof, comprises a variable heavy chain comprising the amino acid sequence of SEQ ID NO: 240; and a variable light chain comprising an amino acid sequence of SEQ ID NO: 328. In some embodiments, an anti-PD-1 antibody, or an antigen-binding fragment thereof, comprises a variable heavy chain comprising the amino acid sequence of SEQ ID NO: 233; and a variable light chain comprising an amino acid sequence of SEQ ID NO: 327. In some embodiments, an anti-PD-1 antibody, or an antigen-binding fragment thereof, comprises a variable heavy chain comprising the amino acid sequence of SEQ ID NO: 241; and a variable light chain comprising an amino acid sequence of SEQ ID NO: 360. In some embodiments, an anti-PD-1 antibody, or an antigen-binding fragment thereof, comprises a variable heavy chain comprising the amino acid sequence of SEQ ID NO: 242; and a variable light chain comprising an amino acid sequence of SEQ ID NO: 327. In some embodiments, an anti-PD-1 antibody, or an antigen-binding fragment thereof, comprises a variable heavy chain comprising the amino acid sequence of SEQ ID NO: 243; and a variable light chain comprising an amino acid sequence of SEQ ID NO: 356. In some embodiments, an anti-PD-1 antibody, or an antigen-binding fragment thereof, comprises a variable heavy chain comprising the amino acid sequence of SEQ ID NO: 158; and a variable light chain comprising an amino acid sequence of SEQ ID NO: 361. In some embodiments, an anti-PD-1 antibody, or an antigen-binding fragment thereof, comprises a variable heavy chain comprising the amino acid sequence of SEQ ID NO: 244; and a variable light chain comprising an amino acid sequence of SEQ ID NO: 362. In some embodiments, an anti-PD-1 antibody, or an antigen-binding fragment thereof, comprises a variable heavy chain comprising the amino acid sequence of SEQ ID NO: 230; and a variable light chain comprising an amino acid sequence of SEQ ID NO: 363. In some embodiments, an anti-PD-1 antibody, or an antigen-binding fragment thereof, comprises a variable heavy chain comprising the amino acid sequence of SEQ ID NO: 245; and a variable light chain comprising an amino acid sequence of SEQ ID NO: 364. In some embodiments, an anti-PD-1 antibody, or an antigen-binding fragment thereof, comprises a variable heavy chain comprising the amino acid sequence of SEQ ID NO: 246; and a variable light chain comprising an amino acid sequence of SEQ ID NO: 365. In some embodiments, an anti-PD-1 antibody, or an antigen-binding fragment thereof, comprises a variable heavy chain comprising the amino acid sequence of SEQ ID NO: 244; and a variable light chain comprising an amino acid sequence of SEQ ID NO: 366. In some embodiments, an anti-PD-1 antibody, or an antigen-binding fragment thereof, comprises a variable heavy chain comprising the amino acid sequence of SEQ ID NO: 247; and a variable light chain comprising an amino acid sequence of SEQ ID NO: 367. In some embodiments, an anti-PD-1 antibody, or an antigen-binding fragment thereof, comprises a variable heavy chain comprising the amino acid sequence of SEQ ID NO: 248; and a variable light chain comprising an amino acid sequence of SEQ ID NO: 368. In some embodiments, an anti-PD-1 antibody, or an antigen-binding fragment thereof, comprises a variable heavy chain comprising the amino acid sequence of SEQ ID NO: 249; and a variable light chain comprising an amino acid sequence of SEQ ID NO: 369. In some embodiments, an anti-PD-1 antibody, or an antigen-binding fragment thereof, comprises a variable heavy chain comprising the amino acid sequence of SEQ ID NO: 250; and a variable light chain comprising an amino acid sequence of SEQ ID NO: 348. In some embodiments, an anti-PD-1 antibody, or an antigen-binding fragment thereof, comprises a variable heavy chain comprising the amino acid sequence of SEQ ID NO: 251; and a variable light chain comprising an amino acid sequence of SEQ ID NO: 348. In some embodiments, an anti-PD-1 antibody, or an antigen-binding fragment thereof, comprises a variable heavy chain comprising the amino acid sequence of SEQ ID NO: 252; and a variable light chain comprising an amino acid sequence of SEQ ID NO: 370. In some embodiments, an anti-PD-1 antibody, or an antigen-binding fragment thereof, comprises a variable heavy chain comprising the amino acid sequence of SEQ ID NO: 230; and a variable light chain comprising an amino acid sequence of SEQ ID NO: 371. In some embodiments, an anti-PD-1 antibody, or an antigen-binding fragment thereof, comprises a variable heavy chain comprising the amino acid sequence of SEQ ID NO: 253; and a variable light chain comprising an amino acid sequence of SEQ ID NO: 372. In some embodiments, an anti-PD-1 antibody, or an antigen-binding fragment thereof, comprises a variable heavy chain comprising the amino acid sequence of SEQ ID NO: 254; and a variable light chain comprising an amino acid sequence of SEQ ID NO: 348. In some embodiments, an anti-PD-1 antibody, or an antigen-binding fragment thereof, comprises a variable heavy chain comprising the amino acid sequence of SEQ ID NO: 255; and a variable light chain comprising an amino acid sequence of SEQ ID NO: 373. In some embodiments, an anti-PD-1 antibody, or an antigen-binding fragment thereof, comprises a variable heavy chain comprising the amino acid sequence of SEQ ID NO: 256; and a variable light chain comprising an amino acid sequence of SEQ ID NO: 374. In some embodiments, an anti-PD-1 antibody, or an antigen-binding fragment thereof, comprises a variable heavy chain comprising the amino acid sequence of SEQ ID NO: 245; and a variable light chain comprising an amino acid sequence of SEQ ID NO: 375. In some embodiments, an anti-PD-1 antibody, or an antigen-binding fragment thereof, comprises a variable heavy chain comprising the amino acid sequence of SEQ ID NO: 257; and a variable light chain comprising an amino acid sequence of SEQ ID NO: 348. In some embodiments, an anti-PD-1 antibody, or an antigen-binding fragment thereof, comprises a variable heavy chain comprising the amino acid sequence of SEQ ID NO: 258; and a variable light chain comprising an amino acid sequence of SEQ ID NO: 376. In some embodiments, an anti-PD-1 antibody, or an antigen-binding fragment thereof, comprises a variable heavy chain comprising the amino acid sequence of SEQ ID NO: 158; and a variable light chain comprising an amino acid sequence of SEQ ID NO: 377. In some embodiments, an anti-PD-1 antibody, or an antigen-binding fragment thereof, comprises a variable heavy chain comprising the amino acid sequence of SEQ ID NO: 259; and a variable light chain comprising an amino acid sequence of SEQ ID NO: 348. In some embodiments, an anti-PD-1 antibody, or an antigen-binding fragment thereof, comprises a variable heavy chain comprising the amino acid sequence of SEQ ID NO: 260; and a variable light chain comprising an amino acid sequence of SEQ ID NO: 356. In some embodiments, an anti-PD-1 antibody, or an antigen-binding fragment thereof, comprises a variable heavy chain comprising the amino acid sequence of SEQ ID NO: 261; and a variable light chain comprising an amino acid sequence of SEQ ID NO: 378. In some embodiments, an anti-PD-1 antibody, or an antigen-binding fragment thereof, comprises a variable heavy chain comprising the amino acid sequence of SEQ ID NO: 262; and a variable light chain comprising an amino acid sequence of SEQ ID NO: 379. In some embodiments, an anti-PD-1 antibody, or an antigen-binding fragment thereof, comprises a variable heavy chain comprising the amino acid sequence of SEQ ID NO: 263; and a variable light chain comprising an amino acid sequence of SEQ ID NO: 348. In some embodiments, an anti-PD-1 antibody, or an antigen-binding fragment thereof, comprises a variable heavy chain comprising the amino acid sequence of SEQ ID NO: 264; and a variable light chain comprising an amino acid sequence of SEQ ID NO: 380. In some embodiments, an anti-PD-1 antibody, or an antigen-binding fragment thereof, comprises a variable heavy chain comprising the amino acid sequence of SEQ ID NO: 262; and a variable light chain comprising an amino acid sequence of SEQ ID NO: 370. In some embodiments, an anti-PD-1 antibody, or an antigen-binding fragment thereof, comprises a variable heavy chain comprising the amino acid sequence of SEQ ID NO: 265; and a variable light chain comprising an amino acid sequence of SEQ ID NO: 363. In some embodiments, an anti-PD-1 antibody, or an antigen-binding fragment thereof, comprises a variable heavy chain comprising the amino acid sequence of SEQ ID NO: 158; and a variable light chain comprising an amino acid sequence of SEQ ID NO: 382. In some embodiments, an anti-PD-1 antibody, or an antigen-binding fragment thereof, comprises a variable heavy chain comprising the amino acid sequence of SEQ ID NO: 158; and a variable light chain comprising an amino acid sequence of SEQ ID NO: 383. In some embodiments, an anti-PD-1 antibody, or an antigen-binding fragment thereof, comprises a variable heavy chain comprising the amino acid sequence of SEQ ID NO: 264; and a variable light chain comprising an amino acid sequence of SEQ ID NO: 384. In some embodiments, an anti-PD-1 antibody, or an antigen-binding fragment thereof, comprises a variable heavy chain comprising the amino acid sequence of SEQ ID NO: 264; and a variable light chain comprising an amino acid sequence of SEQ ID NO: 385. In some embodiments, an anti-PD-1 antibody, or an antigen-binding fragment thereof, comprises a variable heavy chain comprising the amino acid sequence of SEQ ID NO: 253; and a variable light chain comprising an amino acid sequence of SEQ ID NO: 386. In some embodiments, an anti-PD-1 antibody, or an antigen-binding fragment thereof, comprises a variable heavy chain comprising the amino acid sequence of SEQ ID NO: 246; and a variable light chain comprising an amino acid sequence of SEQ ID NO: 374. In some embodiments, an anti-PD-1 antibody, or an antigen-binding fragment thereof, comprises a variable heavy chain comprising the amino acid sequence of SEQ ID NO: 266; and a variable light chain comprising an amino acid sequence of SEQ ID NO: 387. In some embodiments, an anti-PD-1 antibody, or an antigen-binding fragment thereof, comprises a variable heavy chain comprising the amino acid sequence of SEQ ID NO: 267; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 268; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 269; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 270; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 271; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 272; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 273; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 274; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 275; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 276; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 277; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 278; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 279; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 280; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 281; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 282; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 283; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 284; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 285; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 286; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 287; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 288; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 289; and a variable light chain comprising an amino acid sequence of SEQ ID NO: 323. In some embodiments, an anti-PD-1 antibody, or an antigen-binding fragment thereof, comprises a variable heavy chain comprising the amino acid sequence of SEQ ID NO: 290; and a variable light chain comprising an amino acid sequence of SEQ ID NO: 388. In some embodiments, an anti-PD-1 antibody, or an antigen-binding fragment thereof, comprises a variable heavy chain comprising the amino acid sequence of SEQ ID NO: 291; and a variable light chain comprising an amino acid sequence of SEQ ID NO: 389. In some embodiments, an anti-PD-1 antibody, or an antigen-binding fragment thereof, comprises a variable heavy chain comprising the amino acid sequence of SEQ ID NO: 292; and a variable light chain comprising an amino acid sequence of SEQ ID NO: 390. In some embodiments, an anti-PD-1 antibody, or an antigen-binding fragment thereof, comprises a variable heavy chain comprising the amino acid sequence of SEQ ID NO: 293; and a variable light chain comprising an amino acid sequence of SEQ ID NO: 391. In some embodiments, an anti-PD-1 antibody, or an antigen-binding fragment thereof, comprises a variable heavy chain comprising the amino acid sequence of SEQ ID NO: 293; and a variable light chain comprising an amino acid sequence of SEQ ID NO: 392. In some embodiments, an anti-PD-1 antibody, or an antigen-binding fragment thereof, comprises a variable heavy chain comprising the amino acid sequence of SEQ ID NO: 294; and a variable light chain comprising an amino acid sequence of SEQ ID NO: 393. In some embodiments, an anti-PD-1 antibody, or an antigen-binding fragment thereof, comprises a variable heavy chain comprising the amino acid sequence of SEQ ID NO: 295; and a variable light chain comprising an amino acid sequence of SEQ ID NO: 394. In some embodiments, an anti-PD-1 antibody, or an antigen-binding fragment thereof, comprises a variable heavy chain comprising the amino acid sequence of SEQ ID NO: 295; and a variable light chain comprising an amino acid sequence of SEQ ID NO: 395. In some embodiments, an anti-PD-1 antibody, or an antigen-binding fragment thereof, comprises a variable heavy chain comprising the amino acid sequence of SEQ ID NO: 296; and a variable light chain comprising an amino acid sequence of SEQ ID NO: 394. In some embodiments, an anti-PD-1 antibody, or an antigen-binding fragment thereof, comprises a variable heavy chain comprising the amino acid sequence of SEQ ID NO: 296; and a variable light chain comprising an amino acid sequence of SEQ ID NO: 395. In some embodiments, an anti-PD-1 antibody, or an antigen-binding fragment thereof, comprises a variable heavy chain comprising the amino acid sequence of SEQ ID NO: 297; and a variable light chain comprising an amino acid sequence of SEQ ID NO: 396. In some embodiments, an anti-PD-1 antibody, or an antigen-binding fragment thereof, comprises a variable heavy chain comprising the amino acid sequence of SEQ ID NO: 290; and a variable light chain comprising an amino acid sequence of SEQ ID NO: 388. In some embodiments, an anti-PD-1 antibody, or an antigen-binding fragment thereof, comprises a variable heavy chain comprising the amino acid sequence of SEQ ID NO: 298; and a variable light chain comprising an amino acid sequence of SEQ ID NO: 391. In some embodiments, an anti-PD-1 antibody, or an antigen-binding fragment thereof, comprises a variable heavy chain comprising the amino acid sequence of SEQ ID NO: 294; and a variable light chain comprising an amino acid sequence of SEQ ID NO: 393. In some embodiments, an anti-PD-1 antibody, or an antigen-binding fragment thereof, comprises a variable heavy chain comprising the amino acid sequence of SEQ ID NO: 295; and a variable light chain comprising an amino acid sequence of SEQ ID NO: 395. In some embodiments, an anti-PD-1 antibody, or an antigen-binding fragment thereof, comprises a variable heavy chain comprising the amino acid sequence of SEQ ID NO: 296; and a variable light chain comprising an amino acid sequence of SEQ ID NO: 395. In some embodiments, an anti-PD-1 antibody, or an antigen-binding fragment thereof, comprises a variable heavy chain comprising the amino acid sequence of SEQ ID NO: 299; and a variable light chain comprising an amino acid sequence of SEQ ID NO: 397. In some embodiments, an anti-PD-1 antibody, or an antigen-binding fragment thereof, comprises a variable heavy chain comprising the amino acid sequence of SEQ ID NO: 300; and a variable light chain comprising an amino acid sequence of SEQ ID NO: 397. In some embodiments, an anti-PD-1 antibody, or an antigen-binding fragment thereof, comprises a variable heavy chain comprising the amino acid sequence of SEQ ID NO: 301; and a variable light chain comprising an amino acid sequence of SEQ ID NO: 398. In some embodiments, an anti-PD-1 antibody, or an antigen-binding fragment thereof, comprises a variable heavy chain comprising the amino acid sequence of SEQ ID NO: 301; and a variable light chain comprising an amino acid sequence of SEQ ID NO: 399. In some embodiments, an anti-PD-1 antibody, or an antigen-binding fragment thereof, comprises a variable heavy chain comprising the amino acid sequence of SEQ ID NO: 302; and a variable light chain comprising an amino acid sequence of SEQ ID NO: 398. In some embodiments, an anti-PD-1 antibody, or an antigen-binding fragment thereof, comprises a variable heavy chain comprising the amino acid sequence of SEQ ID NO: 302; and a variable light chain comprising an amino acid sequence of SEQ ID NO: 399. In some embodiments, an anti-PD-1 antibody, or an antigen-binding fragment thereof, comprises a variable heavy chain comprising the amino acid sequence of SEQ ID NO: 303; and a variable light chain comprising an amino acid sequence of SEQ ID NO: 398. In some embodiments, an anti-PD-1 antibody, or an antigen-binding fragment thereof, comprises a variable heavy chain comprising the amino acid sequence of SEQ ID NO: 303; and a variable light chain comprising an amino acid sequence of SEQ ID NO: 399. In some embodiments, an anti-PD-1 antibody, or an antigen-binding fragment thereof, comprises a variable heavy chain comprising the amino acid sequence of SEQ ID NO: 304; and a variable light chain comprising an amino acid sequence of SEQ ID NO: 400. In some embodiments, an anti-PD-1 antibody, or an antigen-binding fragment thereof, comprises a variable heavy chain comprising the amino acid sequence of SEQ ID NO: 305; and a variable light chain comprising an amino acid sequence of SEQ ID NO: 400. In some embodiments, an anti-PD-1 antibody, or an antigen-binding fragment thereof, comprises a variable heavy chain comprising the amino acid sequence of SEQ ID NO: 306; and a variable light chain comprising an amino acid sequence of SEQ ID NO: 401. In some embodiments, an anti-PD-1 antibody, or an antigen-binding fragment thereof, comprises a variable heavy chain comprising the amino acid sequence of SEQ ID NO: 307; and a variable light chain comprising an amino acid sequence of SEQ ID NO: 402. In some embodiments, an anti-PD-1 antibody, or an antigen-binding fragment thereof, comprises a variable heavy chain comprising the amino acid sequence of SEQ ID NO: 308; and a variable light chain comprising an amino acid sequence of SEQ ID NO: 402. In some embodiments, an anti-PD-1 antibody, or an antigen-binding fragment thereof, comprises a variable heavy chain comprising the amino acid sequence of SEQ ID NO: 309; and a variable light chain comprising an amino acid sequence of SEQ ID NO: 403. In some embodiments, an anti-PD-1 antibody, or an antigen-binding fragment thereof, comprises a variable heavy chain comprising the amino acid sequence of SEQ ID NO: 310; and a variable light chain comprising an amino acid sequence of SEQ ID NO: 403. In some embodiments, an anti-PD-1 antibody, or an antigen-binding fragment thereof, comprises a variable heavy chain comprising the amino acid sequence of SEQ ID NO: 311; and a variable light chain comprising an amino acid sequence of SEQ ID NO: 404. In some embodiments, an anti-PD-1 antibody, or an antigen-binding fragment thereof, comprises a variable heavy chain comprising the amino acid sequence of SEQ ID NO: 311; and a variable light chain comprising an amino acid sequence of SEQ ID NO: 405. In some embodiments, an anti-PD-1 antibody, or an antigen-binding fragment thereof, comprises a variable heavy chain comprising the amino acid sequence of SEQ ID NO: 311; and a variable light chain comprising an amino acid sequence of SEQ ID NO: 406. In some embodiments, an anti-PD-1 antibody, or an antigen-binding fragment thereof, comprises a variable heavy chain comprising the amino acid sequence of SEQ ID NO: 311; and a variable light chain comprising an amino acid sequence of SEQ ID NO: 407. In some embodiments, an anti-PD-1 antibody, or an antigen-binding fragment thereof, comprises a variable heavy chain comprising the amino acid sequence of SEQ ID NO: 312; and a variable light chain comprising an amino acid sequence of SEQ ID NO: 408. In some embodiments, an anti-PD-1 antibody, or an antigen-binding fragment thereof, comprises a variable heavy chain comprising the amino acid sequence of SEQ ID NO: 312; and a variable light chain comprising an amino acid sequence of SEQ ID NO: 409. In some embodiments, an anti-PD-1 antibody, or an antigen-binding fragment thereof, comprises a variable heavy chain comprising the amino acid sequence of SEQ ID NO: 313; and a variable light chain comprising an amino acid sequence of SEQ ID NO: 408. In some embodiments, an anti-PD-1 antibody, or an antigen-binding fragment thereof, comprises a variable heavy chain comprising the amino acid sequence of SEQ ID NO: 313; and a variable light chain comprising an amino acid sequence of SEQ ID NO: 409. In some embodiments, an anti-PD-1 antibody, or an antigen-binding fragment thereof, comprises a variable heavy chain comprising the amino acid sequence of SEQ ID NO: 314; and a variable light chain comprising an amino acid sequence of SEQ ID NO: 410. In some embodiments, an anti-PD-1 antibody, or an antigen-binding fragment thereof, comprises a variable heavy chain comprising the amino acid sequence of SEQ ID NO: 314; and a variable light chain comprising an amino acid sequence of SEQ ID NO: 411. In some embodiments, an anti-PD-1 antibody, or an antigen-binding fragment thereof, comprises a variable heavy chain comprising the amino acid sequence of SEQ ID NO: 315; and a variable light chain comprising an amino acid sequence of SEQ ID NO: 410. In some embodiments, an anti-PD-1 antibody, or an antigen-binding fragment thereof, comprises a variable heavy chain comprising the amino acid sequence of SEQ ID NO: 315; and a variable light chain comprising an amino acid sequence of SEQ ID NO: 411. In some embodiments, an anti-PD-1 antibody, or an antigen-binding fragment thereof, comprises a variable heavy chain comprising the amino acid sequence of SEQ ID NO: 316; and a variable light chain comprising an amino acid sequence of SEQ ID NO: 410. In some embodiments, an anti-PD-1 antibody, or an antigen-binding fragment thereof, comprises a variable heavy chain comprising the amino acid sequence of SEQ ID NO: 316; and a variable light chain comprising an amino acid sequence of SEQ ID NO: 411. In some embodiments, an anti-PD-1 antibody, or an antigen-binding fragment thereof, comprises a variable heavy chain comprising the amino acid sequence of SEQ ID NO: 317; and a variable light chain comprising an amino acid sequence of SEQ ID NO: 412. In some embodiments, an anti-PD-1 antibody, or an antigen-binding fragment thereof, comprises a variable heavy chain comprising the amino acid sequence of SEQ ID NO: 318; and a variable light chain comprising an amino acid sequence of SEQ ID NO: 412. In some embodiments, an anti-PD-1 antibody, or an antigen-binding fragment thereof, comprises a variable heavy chain comprising the amino acid sequence of SEQ ID NO: 319; and a variable light chain comprising an amino acid sequence of SEQ ID NO: 413. In some embodiments, an anti-PD-1 antibody, or an antigen-binding fragment thereof, comprises a variable heavy chain comprising the amino acid sequence of SEQ ID NO: 319; and a variable light chain comprising an amino acid sequence of SEQ ID NO: 391. In some embodiments, an anti-PD-1 antibody, or an antigen-binding fragment thereof, comprises a variable heavy chain comprising the amino acid sequence of SEQ ID NO: 320; and a variable light chain comprising an amino acid sequence of SEQ ID NO: 413. In some embodiments, an anti-PD-1 antibody, or an antigen-binding fragment thereof, comprises a variable heavy chain comprising the amino acid sequence of SEQ ID NO: 320; and a variable light chain comprising an amino acid sequence of SEQ ID NO: 391. In some embodiments, an anti-PD-1 antibody, or an antigen-binding fragment thereof, comprises a variable heavy chain comprising the amino acid sequence of SEQ ID NO: 321; and a variable light chain comprising an amino acid sequence of SEQ ID NO: 413. In some embodiments, an anti-PD-1 antibody, or an antigen-binding fragment thereof, comprises a variable heavy chain comprising the amino acid sequence of SEQ ID NO: 321; and a variable light chain comprising an amino acid sequence of SEQ ID NO: 391. In some embodiments, an anti-PD-1 antibody, or an antigen-binding fragment thereof, comprises a variable heavy chain comprising the amino acid sequence of SEQ ID NO: 322; and a variable light chain comprising an amino acid sequence of SEQ ID NO: 414. In some embodiments, an anti-PD-1 antibody, or an antigen-binding fragment thereof, comprises a variable heavy chain comprising the amino acid sequence of SEQ ID NO: 530; and a variable light chain comprising an amino acid sequence of SEQ ID NO: 531. In some embodiments, an anti-PD-1 antibody, or an antigen-binding fragment thereof, comprises a variable heavy chain comprising the amino acid sequence of SEQ ID NO: 530; and a variable light chain comprising an amino acid sequence of SEQ ID NO: 532. In some embodiments, an anti-PD-1 antibody, or an antigen-binding fragment thereof, comprises a variable heavy chain comprising the amino acid sequence of SEQ ID NO: 530; and a variable light chain comprising an amino acid sequence of SEQ ID NO: 533. In some embodiments, an anti-PD-1 antibody, or an antigen-binding fragment thereof, comprises a variable heavy chain comprising the amino acid sequence of SEQ ID NO: 530; and a variable light chain comprising an amino acid sequence of SEQ ID NO: 534. In some embodiments, an anti-PD-1 antibody, or an antigen-binding fragment thereof, comprises a variable heavy chain comprising the amino acid sequence of SEQ ID NO: 530; and a variable light chain comprising an amino acid sequence of SEQ ID NO: 535. In some embodiments, an anti-PD-1 antibody, or an antigen-binding fragment thereof, comprises a variable heavy chain comprising the amino acid sequence of SEQ ID NO: 530; and a variable light chain comprising an amino acid sequence of SEQ ID NO: 536. In some embodiments, an anti-PD-1 antibody, or an antigen-binding fragment thereof, comprises a variable heavy chain comprising the amino acid sequence of SEQ ID NO: 530; and a variable light chain comprising an amino acid sequence of SEQ ID NO: 537. In some embodiments, an anti-PD-1 antibody, or an antigen-binding fragment thereof, comprises a variable heavy chain comprising the amino acid sequence of SEQ ID NO: 538; and a variable light chain comprising an amino acid sequence of SEQ ID NO: 539. In some embodiments, an anti-PD-1 antibody, or an antigen-binding fragment thereof, comprises a variable heavy chain comprising the amino acid sequence of SEQ ID NO: 540; and a variable light chain comprising an amino acid sequence of SEQ ID NO: 539. In some embodiments, an anti-PD-1 antibody, or an antigen-binding fragment thereof, comprises a variable heavy chain comprising the amino acid sequence of SEQ ID NO: 538; and a variable light chain comprising an amino acid sequence of SEQ ID NO: 542. In some embodiments, an anti-PD-1 antibody, or an antigen-binding fragment thereof, comprises a variable heavy chain comprising the amino acid sequence of SEQ ID NO: 540; and a variable light chain comprising an amino acid sequence of SEQ ID NO: 542. In some embodiments, an anti-PD-1 antibody, or an antigen-binding fragment thereof, comprises a variable heavy chain comprising the amino acid sequence of SEQ ID NO: 530; and a variable light chain comprising an amino acid sequence of SEQ ID NO: 344.

In some embodiments, the anti-PD-1 antibody comprises a heavy chain (HC) and a light chain (LC). In some embodiments, the anti-PD-1 antibody comprises a heavy chain (HC) comprising a VH sequence and Fc sequence of Table 11. In some embodiments, the anti-PD-1 antibody comprises a light chain (LC) comprising a VL sequence and Ck sequence of Table 11. In some embodiments, the anti-PD-1 antibody comprises a heavy chain (HC) having an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of:

(SEQ ID NO: 691)
EVQLLESGGGLVQPGGSLRLSCAASGFTFSDYYMSWVRQAPGKGLEWVSV
ISNSGGSTYYTDSVKGRFTISRDNSKNTLYLQMNSLRAEDTAVYYCTKDI
GMTYFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGEESVFLFPPKPKD
TLMISRTPEVTCVVVDVSHEDPEVKENWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG.

In some embodiments, the anti-PD-1 antibody comprises a light chain (LC) comprising the amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of:

(SEQ ID NO: 692)
QSVLTQPPSASGAPGQRVTISCSGSYSNIGNQYVSWYQQLPGTAPKLLIY
LNSQRPSGVPDRFSGSKSGTSASLAISGLQSEDEADYYCAAWDDLNVWVF
GGGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS.

In some embodiments, the anti-PD-1 antibody comprises a heavy chain (HC) having an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 691; and a light chain (LC) comprising the amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 692.

In some embodiments, the anti-PD-1 antibody comprises a heavy chain (HC) of SEQ ID NO: 691, and a light chain (LC) of SEQ ID NO: 692.

In some embodiments, the anti-PD-1 antibody comprises a VH and VL comprising the CDRs of the amino acid sequence as set forth in PDAB1 to PDAB254. Determining CDRs is routine and can be done according to the Kabat, Chothia, IMGT numbering systems, and the like. In some embodiments, the anti-PD-1 antibody comprises a VH and VL comprising an amino acid sequence that is at least 90%, 91%, 92%, 93%, 94%, 95%, 96%, 97%, 98%, or 99% identical to the VH and VL pairs as set forth in PDAB1 to PDAB254. In some embodiments, the anti-PD-1 antibody comprises a VH and VL comprising an amino acid sequence that is at least 90%, 91%, 92%, 93%, 94%, 95%, 96%, 97%, 98%, or 99% identical to the VH and VL pairs as set forth in PDAB1 to PDAB254, provided that the CDRs are identical to PDAB1 to PDAB254. These can be collectively referred to as a variant of the VH and VL sequences provided for herein. In some embodiments, the anti-PD-1 antibody comprises the CDR sequences according to the Kabat numbering system, provided in Table 12.

TABLE 12

| VH | HCDR1 | HCDR2 | HCDR3 | VL | LCDR1 | LCDR2 | LCDR3 |
|---|---|---|---|---|---|---|---|
| EVQLLESGGGLVQPG | SDAMN | TIYSG | GGNFY | EIVMTQSPATLSVSPG | RASQS | GASTR | QQYNN |
| GSLRLSCAASGFTFS | (SEQ | GSTYY | NYFDY | ERATLSCRASQSVSSN | VSSNL | AT | WPPWT |
| SDAMNWVRQAPGKGL | ID | ADSVK | (SEQ | LAWYQQKPGQAPRLLI | A | (SEQ | (SEQ |
| EWVSTIYSGGSTYYA | NO: | G | ID | YGASTRATGIPARFSG | (SEç | ID | ID |
| DSVKGRFTISRDNSK | 547) | (SEQ | NO: | SGSGTEFTLTISSLQS | ID | NO: | NO: |
| NTLYLQMNSLRAEDT | | ID | 549) | EDFAVYYCQQYNNWPP | NO: | 560) | 561) |
| AVYYCARGGNFYNYF | | NO: | | WTFGQGTKVEIK (SEQ | 559) | | |
| DYWGQGTLVTVSS | | 548) | | ID NO: 323) | | | |
| (SEQ ID NO: 136) | | | | | | | |
| EVQLLESGGGLVQPG | DHYMS | TISSG | ILKNG | EIVMTQSPATLSVSPG | RASQS | GASTR | QQYNN |
| GSLRLSCAASGFTFS | (SEQ | GNYIY | KYIYY | ERATLSCRASQSVSSN | VSSNL | AT | WPPWT |
| DHYMSWVRQAPGKGL | ID | YADSV | FDY | LAWYQQKPGQAPRLLI | A | (SEQ | (SEQ |
| EWVSTISSGGNYIYY | NO: | KG | (SEQ | YGASTRATGIPARFSG | (SEQ | ID | ID |
| ADSVKGRFTISRDSS | 550) | (SEQ | ID | SGSGTEFTLTISSLQS | ID | NO: | NO: |
| KNTLYLQMNSLRAED | | ID | NO: | EDFAVYYCQQYNNWPP | NO: | 560) | 561) |
| TAVYYCARILKNGKY | | NO: | 552) | WTFGQGTKVEIK (SEQ | 559) | | |
| IYYFDYWGQGTLVTV | | 551) | | ID NO: 323) | | | |
| SS (SEQ ID NO: | | | | | | | |
| 156) | | | | | | | |
| EVQLLESGGAFVQPG | DYYMS | VISNS | DIGMT | QSVLTQPPSASGAPGQ | SGSYS | LNSQR | AAWDD |
| GSLRLSCAASGFTFS | (SEQ | GGSTY | YFDY | RVTISCSGSYSNIGNS | NIGNS | PS | LNVWV |
| DYYMSWVRQAPGKGL | ID | YTDSV | (SEQ | YVSWYQQLPGTAPKLL | YVS | (SEQ | (SEQ |
| EWVSVISNSGGSTYY | NO: | KG | ID | IYLNSQRPSGVPDRES | (SEQ | ID | ID |
| TDSVKGRFTISRDNS | 553) | (SEQ | NO: | GSKSGTSASLAISGLQ | ID | NO: | NO: |
| KNTLYLQINSLRAED | | ID | 555) | SEDEADYYCAAWDDLN | NO: | 563) | 564) |
| TAVYYCTKDIGMTYF | | NO: | | VWVFGGGTKLTVL | 562) | | |
| DYWGQGTLVTVSS | | 554) | | (SEQ ID NO: 344) | | | |
| (SEQ ID NO: 187) | | | | | | | |
| QVTLKESGPGILQPS | TFGMG | HIWWD | IITTA | QAVVTQESALTTSPGE | RSSTG | GTNNR | ALWYS |
| QTLSLTCSFSGFSLS | VG | DDKYY | WYFDV | TVTLTCRSSTGAVTTS | AVTTS | AP | NHFI |
| TFGMGVGWIRQPSGK | (SEQ | NPALK | (SEQ | NYANWVQEKPDHLFTG | NYAN | (SEQ | (SEQ |
| GLEWLAHIWWDDDKY | ID | S | ID | LIGGTNNRAPGVPARF | (SEQ | ID | ID |
| YNPALKSRLTISKDT | NO: | (SEQ | NO: | SGSLIGDKAALTITGA | ID | NO: | NO: |
| SKNQVFLKIANVDTA | 556) | ID | 558) | QTEDEAIYFCALWYSN | NO: | 566) | 567) |
| DTATYYCARIITTAW | | NO: | | HFIFGSGTKVTVL | 565) | | |
| YFDVWGTGTTVTVSS | | 557) | | (SEQ ID NO: 388) | | | |
| (SEQ ID NO: 290) | | | | | | | |
| EVQLLESGGGLVQPG | DYYMS | VISNS | DIGMT | QSVLTQPPSASGAPGQ | SGSYS | LNSQR | AAWDD |
| GSLRLSCAASGFTFS | (SEQ | GGSTY | YFDY | RVTISCSGSYSNIGES | NIGES | PS | LNVWV |
| DYYMSWVRQAPGKGL | ID | YTDSV | (SEQ | YVSWYQQLPGTAPKLL | YVS | (SEQ | (SEQ |
| EWVSVISNSGGSTYY | NO: | KG | ID | IYLNSQRPSGVPDRES | (SEQ | ID | ID |
| TDSVKGRFTISRDNS | 553) | (SEQ | NO: | GSKSGTSASLAISGLQ | ID | NO: | NO: |
| KNTLYLQMNSLRAED | | ID | 555) | SEDEADYYCAAWDDLN | NO: | 563) | 564) |
| TAVYYCTKDIGMTYF | | NO: | | VWVFGGGTKLTVL | 568) | | |
| DYWGQGTLVTVSS | | 554) | | (SEQ ID NO: 531) | | | |
| (SEQ ID NO: 530) | | | | | | | |
| EVQLLESGGGLVQPG | DYYMS | VISNS | DIGMT | QSVLTQPPSASGAPGQ | SGSYS | LNSQR | AAWDD |
| GSLRLSCAASGFTFS | (SEç | GGSTY | YFDY | RVTISCSGSYSNIGGS | NIGGS | PS | LNVWV |
| DYYMSWVRQAPGKGL | ID | YTDSV | (SEQ | YVSWYQQLPGTAPKLL | YVS | (SEQ | (SEQ |
| EWVSVISNSGGSTYY | NO: | KG | ID | IYLNSQRPSGVPDRES | (SEQ | ID | ID |
| TDSVKGRFTISRDNS | 553) | (SEQ | | GSKSGTSASLAISGLQ | ID | | |
| KNTLYLQMNSLRAED | | ID | NO: | SEDEADYYCAAWDDLN | NO: | NO: | NO: |
| TAVYYCTKDIGMTYF | | NO: | 555) | VWVFGGGTKLTVL | 569) | 563) | 564) |
| DYWGQGTLVTVSS | | 554) | | (SEQ ID NO: 532) | | | |
| (SEQ ID NO: 530) | | | | | | | |
| EVQLLESGGGLVQPG | DYYMS | VISNS | DIGMT | QSVLTQPPSASGAPGQ | SGSYS | LNSQR | AAWDD |
| GSLRLSCAASGFTFS | (SEQ | GGSTY | YFDY | RVTISCSGSYSNIGLS | NIGLS | PS | LNVWV |
| DYYMSWVRQAPGKGL | ID | YTDSV | (SEQ | YVSWYQQLPGTAPKLL | YVS | (SEQ | (SEQ |
| EWVSVISNSGGSTYY | NO: | KG | ID | IYLNSQRPSGVPDRES | (SEQ | ID | ID |
| TDSVKGRFTISRDNS | 553) | (SEQ | NO: | GSKSGTSASLAISGLQ | ID | NO: | NO: |
| KNTLYLQMNSLRAED | | ID | 555) | SEDEADYYCAAWDDLN | NO: | 563) | 564) |
| TAVYYCTKDIGMTYF | | NO: | | VWVFGGGTKLTVL | 570) | | |
| DYWGQGTLVTVSS | | 554) | | (SEQ ID NO: 533) | | | |
| (SEQ ID NO: 530) | | | | | | | |
| EVQLLESGGGLVQPG | DYYMS | VISNS | DIGMT | QSVLTOPPSASGAPGQ | SGSYS | LNSQR | AAWDD |
| GSLRLSCAASGFTFS | (SEQ | GGSTY | YFDY | RVTISCSGSYSNIGQS | NIGQS | PS | LNVWV |
| DYYMSWVRQAPGKGL | ID | YTDSV | (SEQ | YVSWYQQLPGTAPKLL | YVS | (SEQ | (SEQ |
| EWVSVISNSGGSTYY | NO: | KG | ID | IYLNSQRPSGVPDRFS | (SEQ | ID | ID |
| TDSVKGRFTISRDNS | 553) | (SEQ | NO: | GSKSGTSASLAISGLQ | ID | NO: | NO: |
| KNTLYLQMNSLRAED | | ID | 555) | SEDEADYYCAAWDDLN | NO: | 563) | 564) |
| TAVYYCTKDIGMTYF | | NO: | | VWVFGGGTKLTVL | 571) | | |
| DYWGQGTLVTVSS | | 554) | | (SEQ ID NO: 534) | | | |
| (SEQ ID NO: 530) | | | | | | | |
| EVQLLESGGGLVQPG | DYYMS | VISNS | DIGMT | QSVLTQPPSASGAPGQ | SGSYS | LNSQR | AAWDD |
| GSLRLSCAASGFTFS | (SEQ | GGSTY | YFDY | RVTISCSGSYSNIGSS | NIGSS | PS | LNVWV |
| DYYMSWVRQAPGKGL | ID | YTDSV | (SEQ | YVSWYQQLPGTAPKLL | YVS | (SEQ | (SEQ |
| EWVSVISNSGGSTYY | NO: | KG | ID | IYLNSQRPSGVPDRFS | (SEQ | ID | ID |
| TDSVKGRFTISRDNS | 553) | (SEQ | NO: | GSKSGTSASLAISGLQ | ID | NO: | NO: |
| KNTLYLQMNSLRAED | | ID | 555) | SEDEADYYCAAWDDLN | NO: | 563) | 564) |
| TAVYYCTKDIGMTYF | | NO: | | VWVFGGGTKLTVL | 572) | | |
| DYWGQGTLVTVSS | | 554) | | (SEQ ID NO: 535) | | | |
| (SEQ ID NO: 530) | | | | | | | |
| EVQLLESGGGLVQPG | DYYMS | VISNS | DIGMT | QSVLTQPPSASGAPGQ | SGSYS | LNSQR | AAWDD |
| GSLRLSCAASGFTFS | (SEQ | GGSTY | YFDY | RVTISCSGSYSNIGND | NIGND | PS | LNVWV |
| DYYMSWVRQAPGKGL | ID | YTDSV | (SEQ | YVSWYQQLPGTAPKLL | YVS | (SEQ | (SEQ |
| EWVSVISNSGGSTYY | NO: | KG | ID | IYLNSQRPSGVPDRES | (SEQ | ID | ID |
| TDSVKGRFTISRDNS | 553) | (SEQ | NO: | GSKSGTSASLAISGLQ | ID | NO: | NO: |
| KNTLYLQMNSLRAED | | ID | 555) | SEDEADYYCAAWDDLN | NO: | 563) | 564) |
| TAVYYCTKDIGMTYF | | NO: | | VWVFGGGTKLTVL | 573) | | |
| DYWGQGTLVTVSS | | 554) | | (SEQ ID NO: 536) | | | |
| (SEQ ID NO: 530) | | | | | | | |
| EVQLLESGGGLVQPG | DYYMS | VISNS | DIGMT | QSVLTOPPSASGAPGQ | SGSYS | LNSQR | AAWDD |
| GSLRLSCAASGFTFS | (SEQ | GGSTY | YFDY | RVTISCSGSYSNIGNQ | NIGNQ | PS | LNVWV |
| DYYMSWVRQAPGKGL | ID | YTDSV | (SEQ | YVSWYQQLPGTAPKLL | YVS | (SEQ | (SEQ |
| EWVSVISNSGGSTYY | NO: | KG | ID | IYLNSQRP SGVPDRFS | (SEQ | ID | ID |
| TDSVKGRFTISRDNS | 553) | (SEQ | NO: | GSKSGTSASLAISGLQ | ID | NO: | NO: |
| KNTLYLQMNSLRAED | | ID | 555) | SEDEADYYCAAWDDLN | NO: | 563) | 564) |
| TAVYYCTKDIGMTYF | | NO: | | VWVFGGGTKLTVL | 574) | | |
| DYWGQGTLVTVSS | | 554) | | (SEQ ID NO: 537) | | | |
| (SEQ ID NO: 530) | | | | | | | |
| QVTLKESGPTLVKPT | TFGMG | HIWWD | IITTA | QAVVTQEPSLTVSPGG | RSSTG | GTNNR | ALWYS |
| QTLTLTCTFSGFSLS | VG | DDKYY | WYFDV | TVTLTCRSSTGAVTTS | AVTTS | AP | NHFI |
| TFGMGVGWIRQPPGK | (SEQ | NPALK | (SEQ | NYANWVQQKPGQAFRG | NYAN | (SEQ | (SEQ |
| ALEWLAHIWWDDDKY | ID | S | ID | LIGGTNNRAPGVPDRF | (SEQ | ID | ID |
| YNPALKSRLTITKDT | NO: | (SEQ | NO: | SGSILGNKAALTI TGA | ID | NO: | NO: |
| SKNQVVLTMTNMDPV | 556) | ID | 558) | QADDESIYFCALWYSN | NO: | 566) | 567) |
| DTATYYCARIITTAW | | NO: | | HFIFGSGTKVTVL | 565) | | |
| YFDVWGTGTTVTVSS | | 557) | | (SEQ ID NO: 539) | | | |
| (SEQ ID NO: 538) | | | | | | | |
| QVTLKESGPTLVKPT | TFGMG | HIWWD | IITTA | QAVVTQEPSLTVSPGG | RSSTG | GTNNR | ALWYS |
| QTLTLTCSFSGFSLS | VG | DDKYY | WYFDV | TVTLTCRSSTGAVTTS | AVTTS | AP | NHFI |
| TFGMGVGWIRQPPGK | (SEQ | NPALK | (SEQ | NYANWVQQKPGQAFRG | NYAN | (SEQ | (SEQ |
| GLEWIGHIWWDDDKY | ID | S | ID | LIGGINNRAPGVPDRF | (SEQ | ID | ID |
| YNPALKSRLTITKDT | NO: | (SEQ | NO: | SGSILGNKAALTITGA | ID | NO: | NO: |
| SKNQVVLTMTNMDPV | 556) | ID | 558) | QADDESIYFCALWYSN | NO: | 566) | 567) |
| DTATYYCARIITTAW | | NO: | | HFIFGSGTKVTVL | 565) | | |
| YFDVWGTGTTVTVSS | | 557) | | (SEQ ID NO: 539) | | | |
| (SEQ ID NO: 540) | | | | | | | |
| QVTLKESGPTLVKPT | TFGMG | HIWWD | IITTA | QAVVTQEPSLTVSPGG | RSSTG | GTNNR | ALWYS |
| QTLTLTCSFSGFSLS | VG | DDKYY | WYFDV | TVTLTCRSSTGAVTTS | AVTTS | AP | NHFI |
| TFGMGVGWIRQPPGK | (SEQ | NPALK | (SEQ | NYANWVQQKPGQAFRG | NYAN | (SEQ | (SEQ |
| GLEWIGHIWWDDDKY | ID | S | ID | LIGGTNNRAPGVPDRF | (SEQ | ID | ID |
| YNPALKSRLTITKDT | NO: | (SEQ | NO: | SGSLIGDKAALTITGA | ID | NO: | NO: |
| SKNQVVLTMTNMDPV | 556) | ID | 558) | QADDESDYYCALWYSN | NO: | 566) | 567) |
| DTATYYCARIITTAW | | NO: | | HFIFGSGTKVTVL | 565) | | |
| YFDVWGTGTTVTVSS | | 557) | | (SEQ ID NO: 541) | | | |
| (SEQ ID NO: 540) | | | | | | | |
| QVTLKESGPTLVKPT | TFGMG | HIWWD | IITTA | QAVVTQEPSLTVSPGG | RSSTG | GTNNR | ALWYS |
| QTLTLTCTFSGFSLS | VG | DDKYY | WYFDV | TVTLTCRSSTGAVTTS | AVTTS | AP | NHFI |
| TFGMGVGWIRQPPGK | (SEQ | NPALK | (SEQ | NYANWVQQKPGQAFRG | NYAN | (SEQ | (SEQ |
| ALEWLAHIWWDDDKY | ID | S | ID | LIGGTNNRAPGVPDRF | (SEQ | ID | ID |
| YNPALKSRLTITKDT | NO: | (SEQ | NO: | SGSILGNKAALTI TGA | ID | NO: | NO: |
| SKNQVVLTMTNMDPV | 556) | ID | 558) | QADDESDYYCALWYSN | NO: | 566) | 567) |
| DTATYYCARIITTAW | | NO: | | HFIFGSGTKVTVL | 565) | | |
| YFDVWGTGTTVTVSS | | 557) | | (SEQ ID NO: 542) | | | |
| (SEQ ID NO: 538) | | | | | | | |
| QVTLKESGPTLVKPT | TFGMG | HIWWD | IITTA | QAVVTQEPSLTVSPGG | RSSTG | GTNNR | ALWYS |
| QTLTLTCSFSGFSLS | VG | DDKYY | WYFDV | TVTLTCRSSTGAVTTS | AVTTS | AP | NHFI |
| TFGMGVGWIRQPPGK | (SEQ | NPALK | (SEQ | NYANWVQQKPGQAFRG | NYAN | (SEQ | (SEQ |
| GLEWIGHIWWDDDKY | ID | S | ID | LIGGTNNRAPGVPDRF | (SEQ | ID | ID |
| YNPALKSRLTITKDT | NO: | (SEQ | NO: | SGSILGNKAALTI TGA | ID | NO: | NO: |
| SKNQVVLTMTNMDPV | 556) | ID | 558) | QADDESDYYCALWYSN | NO: | 566) | 567) |
| DTATYYCARIITTAW | | NO: | | HFIFGSGTKVTVL | 565) | | |
| YFDVWGTGTTVTVSS | | 557) | | (SEQ ID NO: 542) | | | |
| (SEQ ID NO: 540) | | | | | | | |
| EVQLLESGGGLVQPG | DYYMS | VISNS | DIGMT | QSVLTOPPSASGAPGQ | SGSYS | LNSQR | AAWDD |
| GSLRLSCAASGFTFS | (SEQ | GGSTY | YFDY | RVTISCSGSYSNIGNS | NIGNS | PS | LNVWV |
| DYYMSWVRQAPGKGL | ID | YTDSV | (SEQ | YVSWYQQLPGTAPKLL | YVS | (SEQ | (SEQ |
| EWVSVISNSGGSTYY | NO: | KG | ID | IYLNSQRPSGVPDRES | (SEQ | ID | ID |
| TDSVKGRFTISRDNS | 553) | (SEQ | NO: | GSKSGTSASLAISGLQ | ID | NO: | NO: |
| KNTLYLQMNSLRAED | | ID | 555) | SEDEADYYCAAWDDLN | NO: | 563) | 564) |
| TAVYYCTKDIGMTYF | | NO: | | VWVFGGGTKLTVL | 562) | | |
| DYWGQGTLVTVSS | | 554) | | (SEQ ID NO: 344) | | | |
| (SEQ ID NO: 530) | | | | | | | |
| EVQLLESGGAFVQPG | DYYMS | VISNS | DIGMT | QSVLTQPPSASGAPGQ | SGSYS | LNSQR | AAWDD |
| GSLRLSCAASGFTFS | (SEQ | GGSTY | YFDY | RVTISCSGSYSNIGNS | NIGNS | PS | LNVWV |
| DYYMSWVRQAPGKGL | ID | YTDSV | (SEQ | YVSWYQQLPGTAPKLL | YVS | (SEQ | (SEQ |
| EWVSVISNSGGSTYY | NO: | KG | ID | IYLNSQRPSGVPDRES | (SEQ | ID | ID |
| TDSVKGRFTISRDNS | 553) | (SEQ | NO: | GSKSGTSASLAISGLQ | ID | NO: | NO: |
| KNTLYLQMNSLRAED | | ID | 555) | SEDEADYYCAAWDDLN | NO: | 563) | 564) |
| TAVYYCTKDIGMTYF | | NO: | | VWVFGGGTKLTVL | 562) | | |
| DYWGQGTLVTVSS | | 554) | | (SEQ ID NO: 344) | | | |
| (SEQ ID NO: 183) | | | | | | | |

In some embodiments, the anti-PD-1 antibody comprises a CDR1 from any one of CDR1 of Table 12, a CDR2 from any one of CDR2 of Table 12, and a CDR3 from any one of CDR3 of Table 12. In some embodiments, the anti-PD-1 antibody comprises a variable heavy (VH) chain comprising a heavy chain CDR1 (HCDR1) from any one of HCDR1 of Table 12, a heavy chain CDR2 (HCDR2) from any one of HCDR2 of Table 12, and a heavy chain CDR3 (HCDR3) from any one of HCDR3 of Table 12. In some embodiments, the anti-PD-1 antibody comprises a variable light (VL) chain comprising a light chain CDR1 (LCDR1) from any one of LCDR1 of Table 12, a light chain CDR2 (LCDR2) from any one of LCDR2 of Table 12, and a light chain CDR3 (LCDR3) from any one of LCDR3 of Table 12. In some embodiments, the amino acid residues of the CDRs shown above contain mutations. In some embodiments, the CDRs contain 1, 2, 3, 4, 5, 6, 7, 8, 9, or 10 substitutions or mutations. In some embodiments, the substitution is a conservative substitution.

In some embodiments, the anti-PD-1 antibody comprises the VH comprising the HCDR1 having an amino acid sequence of any one SEQ ID NOs: 547, 550, 553, or 556, the HCDR2 having an amino acid sequence of any one of SEQ ID NOs: 548, 551, 554, or 557, and the HCDR3 having an amino acid sequence of any one of SEQ ID NOs: 549, 552, 555, or 558; and the VL comprising the LCDR1 having an amino acid sequence of any one SEQ ID NOs: 559, 562, 565, 568, 569, 570, 571, 572, 573, or 574, the LCDR2 having an amino acid sequence of any one of SEQ ID NOs: 560, 563, or 566, and the LCDR3 having an amino acid sequence of any one of SEQ ID NOs: 561, 564, or 567. In some embodiments, the anti-PD-1 antibody comprises the VH of any one of SEQ ID NOs: 136, 156, 183, 187, 290, 530, 538, or 540, comprising the HCDR1 having an amino acid sequence of any one SEQ ID NOs: 547, 550, 553, or 556, the HCDR2 having an amino acid sequence of any one of SEQ ID NOs: 548, 551, 554, or 557, and the HCDR3 having an amino acid sequence of any one of SEQ ID NOs: 549, 552, 555, or 558; and the VL of any one of SEQ ID NOs: 323, 344, 388, 531, 532, 533, 534, 535, 536, 537, 539, 541, or 542, comprising the LCDR1 having an amino acid sequence of any one SEQ ID NOs: 559, 562, 565, 568, 569, 570, 571, 572, 573, or 574, the LCDR2 having an amino acid sequence of any one of SEQ ID NOs: 560, 563, or 566, and the LCDR3 having an amino acid sequence of any one of SEQ ID NOs: 561, 564, or 567.

In some embodiments, the anti-PD-1 antibody comprises the VH comprising the HCDR1 having an amino acid sequence of SEQ ID NO: 547, the HCDR2 having an amino acid sequence of SEQ ID NO: 548, and the HCDR3 having an amino acid sequence of SEQ ID NO: 549; and the VL comprising the LCDR1 having an amino acid sequence of SEQ ID NO: 559, the LCDR2 having an amino acid sequence of any one of SEQ ID NO: 560, and the LCDR3 having an amino acid sequence of SEQ ID NO: 561. In some embodiments, the anti-PD-1 antibody comprises the VH comprising the HCDR1 having an amino acid sequence of SEQ ID NO: 550, the HCDR2 having an amino acid sequence of SEQ ID NO: 551, and the HCDR3 having an amino acid sequence of SEQ ID NO: 552; and the VL comprising the LCDR1 having an amino acid sequence of SEQ ID NO: 559, the LCDR2 having an amino acid sequence of any one of SEQ ID NO: 560, and the LCDR3 having an amino acid sequence of SEQ ID NO: 561. In some embodiments, the anti-PD-1 antibody comprises the VH comprising the HCDR1 having an amino acid sequence of SEQ ID NO: 553, the HCDR2 having an amino acid sequence of SEQ ID NO: 554, and the HCDR3 having an amino acid sequence of SEQ ID NO: 555; and the VL comprising the LCDR1 having an amino acid sequence of SEQ ID NO: 562, the LCDR2 having an amino acid sequence of any one of SEQ ID NO: 563, and the LCDR3 having an amino acid sequence of SEQ ID NO: 564. In some embodiments, the anti-PD-1 antibody comprises the VH comprising the HCDR1 having an amino acid sequence of SEQ ID NO: 556, the HCDR2 having an amino acid sequence of SEQ ID NO: 567, and the HCDR3 having an amino acid sequence of SEQ ID NO: 568; and the VL comprising the LCDR1 having an amino acid sequence of SEQ ID NO: 565, the LCDR2 having an amino acid sequence of any one of SEQ ID NO: 566, and the LCDR3 having an amino acid sequence of SEQ ID NO: 567. In some embodiments, the anti-PD-1 antibody comprises the VH comprising the HCDR1 having an amino acid sequence of SEQ ID NO: 553, the HCDR2 having an amino acid sequence of SEQ ID NO: 554, and the HCDR3 having an amino acid sequence of SEQ ID NO: 555; and the VL comprising the LCDR1 having an amino acid sequence of SEQ ID NO: 568, the LCDR2 having an amino acid sequence of any one of SEQ ID NO: 563, and the LCDR3 having an amino acid sequence of SEQ ID NO: 564. In some embodiments, the anti-PD-1 antibody comprises the VH comprising the HCDR1 having an amino acid sequence of SEQ ID NO: 553, the HCDR2 having an amino acid sequence of SEQ ID NO: 554, and the HCDR3 having an amino acid sequence of SEQ ID NO: 555; and the VL comprising the LCDR1 having an amino acid sequence of SEQ ID NO: 569, the LCDR2 having an amino acid sequence of any one of SEQ ID NO: 563, and the LCDR3 having an amino acid sequence of SEQ ID NO: 564. In some embodiments, the anti-PD-1 antibody comprises the VH comprising the HCDR1 having an amino acid sequence of SEQ ID NO: 553, the HCDR2 having an amino acid sequence of SEQ ID NO: 554, and the HCDR3 having an amino acid sequence of SEQ ID NO: 555; and the VL comprising the LCDR1 having an amino acid sequence of SEQ ID NO: 570, the LCDR2 having an amino acid sequence of any one of SEQ ID NO: 563, and the LCDR3 having an amino acid sequence of SEQ ID NO: 564. In some embodiments, the anti-PD-1 antibody comprises the VH comprising the HCDR1 having an amino acid sequence of SEQ ID NO: 553, the HCDR2 having an amino acid sequence of SEQ ID NO: 554, and the HCDR3 having an amino acid sequence of SEQ ID NO: 555; and the VL comprising the LCDR1 having an amino acid sequence of SEQ ID NO: 571, the LCDR2 having an amino acid sequence of any one of SEQ ID NO: 563, and the LCDR3 having an amino acid sequence of SEQ ID NO: 564. In some embodiments, the anti-PD-1 antibody comprises the VH comprising the HCDR1 having an amino acid sequence of SEQ ID NO: 553, the HCDR2 having an amino acid sequence of SEQ ID NO: 554, and the HCDR3 having an amino acid sequence of SEQ ID NO: 555; and the VL comprising the LCDR1 having an amino acid sequence of SEQ ID NO: 572, the LCDR2 having an amino acid sequence of any one of SEQ ID NO: 563, and the LCDR3 having an amino acid sequence of SEQ ID NO: 564. In some embodiments, the anti-PD-1 antibody comprises the VH comprising the HCDR1 having an amino acid sequence of SEQ ID NO: 553, the HCDR2 having an amino acid sequence of SEQ ID NO: 554, and the HCDR3 having an amino acid sequence of SEQ ID NO: 555; and the VL comprising the LCDR1 having an amino acid sequence of SEQ ID NO: 573, the LCDR2 having an amino acid sequence of any one of SEQ ID NO: 563, and the LCDR3 having an amino acid sequence of SEQ ID NO: 564. In some embodiments, the anti-PD-1 antibody comprises the VH comprising the HCDR1 having an amino acid sequence of SEQ ID NO: 553, the HCDR2 having an amino acid sequence of SEQ ID NO: 554, and the HCDR3 having an amino acid sequence of SEQ ID NO: 555; and the VL comprising the LCDR1 having an amino acid sequence of SEQ ID NO: 574, the LCDR2 having an amino acid sequence of any one of SEQ ID NO: 563, and the LCDR3 having an amino acid sequence of SEQ ID NO: 564. In some embodiments, the anti-PD-1 antibody comprises the VH comprising the HCDR1 having an amino acid sequence of SEQ ID NO: 556, the HCDR2 having an amino acid sequence of SEQ ID NO: 557, and the HCDR3 having an amino acid sequence of SEQ ID NO: 558; and the VL comprising the LCDR1 having an amino acid sequence of SEQ ID NO: 565, the LCDR2 having an amino acid sequence of any one of SEQ ID NO: 566, and the LCDR3 having an amino acid sequence of SEQ ID NO: 567. In some embodiments, the anti-PD-1 antibody comprises the VH comprising the HCDR1 having an amino acid sequence of SEQ ID NO: 553, the HCDR2 having an amino acid sequence of SEQ ID NO: 554, and the HCDR3 having an amino acid sequence of SEQ ID NO: 555; and the VL comprising the LCDR1 having an amino acid sequence of SEQ ID NO: 562, the LCDR2 having an amino acid sequence of any one of SEQ ID NO: 563, and the LCDR3 having an amino acid sequence of SEQ ID NO: 564.

In some embodiments, the anti-PD-1 antibody comprises a VH having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 530, 538, and 540, provided that the VH comprises a HCDR1 having an amino acid sequence of any one SEQ ID NOs: 547, 550, 553, or 556, a HCDR2 having an amino acid sequence of any one SEQ ID NOs: 548, 551, 554, or 557, and a HCDR3 having an amino acid sequence of any one SEQ ID NOs: 549, 552, 555, or 558; and

In some embodiments, the anti-PD-1 antibody comprises a VL having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 531, 532, 533, 534, 535, 536, 537, 539, 541, and 542, provided that the VL comprises a LCDR1 having an amino acid sequence of any one SEQ ID NOs: 559, 562, 565, 568, 569, 570, 571, 572, 573, or 574, a LCDR2 having an amino acid sequence of any one SEQ ID NOs: 560, 563, or 566, and a LCDR3 having an amino acid sequence of any one SEQ ID NOs: 561, 564, or 567.

In some embodiments, the antibodies comprise a VH or VL that comprises a glutamine as the N-terminal residue. In some embodiments, the antibodies comprise a VH or VL that comprises a glutamic acid (E) residue as the N-terminal residue. In some embodiments, antibodies are provided wherein the glutamine (Q) is mutated to a glutamic acid (E) residue.

As provided for herein, in some embodiments, the inhibitory receptor effector domain binds to LAG-3. In some embodiments, the antibody is as described in Angin et al., J Immunol Feb. 15, 2020, 204 (4) 810-818; KR20180004094A, EP3798234A1, and KR20180021833A, each of which is hereby incorporated by reference in its entirety.

### Anti-FcγRIIβ Antibodies

In some embodiments, the effector domain is an antibody that selectively binds to FcγRIIb. Such an antibody can also be referred to as an effector domain or FcγRII binding effector domain. The antibody can be linked to a Fc polypeptide (domain), such as those provided for herein, including those that are specifically bind to FcγRIIb or those that are effectorless. The antibody can also be linked to an anti-PD-1 antibody, such as those provided for herein. The anti-PD-1 antibody can be an agonist antibody. Thus, in some embodiments, the antibody is a bi-specific antibody in that it has at two antigen binding domains that bind to different antigens, such as PD-1 and FcγRIIb. In some embodiments, the anti-FcγRIIb antibody is not linked to a anti-PD-1 antibody. In some embodiments, the anti-FcγRIIβ antibody, or antigen-binding fragment thereof, is in a scFv, Fab, Fab′, F(ab′)2, and/or VHH antibody format. In some embodiments, the antibody, or antigen-binding fragment thereof, is in a scFv format. In some embodiments, the antibody, or antigen-binding fragment thereof, is in a Fab format. In some embodiments, the antibody, or antigen-binding fragment thereof, is in a Fab′ format. In some embodiments, the antibody, or antigen-binding fragment thereof, is in a F(ab′)2 format. In some embodiments, the antibody, or antigen-binding fragment thereof, is in a VHH format. In some embodiments, the anti-FcγRIIb antibody is an IgG format or scFv or a format as provided for herein.

In some embodiments, the C-terminus of the Fc polypeptide is linked to the N-terminus of the anti-FcγRIIb antibody. In some embodiments, the N-terminus of the Fc polypeptide is linked to a C-terminus of a polypetide of the anti-FcγRIIb antibody, such as the heavy variable chain of such antibody. In some embodiments, the C-terminus of the Fc polypeptide is linked to a N-terminus of a polypetide of the anti-FcγRIIb antibody, such as the heavy variable chain of such antibody. In some embodiments, the Fc polypeptide is linked to the anti-FcγRIIb antibody, such as without a peptide linker. In some embodiments, the Fc polypeptide is linked to the anti-FcγRIIb antibody through a peptide linker. Non-limiting examples of such linkers are provided for herein.

In some embodiments, the anti-FcγRIIβ antibody comprises a polypeptide comprising an amino acid sequence comprising any one variable heavy chains and any one variable light chains provided in Table 6 below. In some embodiments, the anti-FcγRIIβ antibody is an scFv comprising an amino acid sequence comprising any one variable heavy chains and any one variable light chains provided in Table 6 below. In some embodiments, the anti-FcγRIIβ antibody is in an scFv format. In some embodiments, the anti-FcγRIIβ scFv antibody is as provided in Table 6. In some embodiments, the anti-FcγRIIβ scFv antibody comprises a VH linked or conjugated to a VL. In some embodiments, the anti-FcγRIIβ scFv antibody comprises a VH linked or conjugated to a VL, wherein the VH and the VL are linked or conjugated from a C-terminus of the VH to an N-terminus of the VL. In some embodiments, the the anti-FcγRIIβ scFv antibody comprises a VH linked or conjugated to a VL, wherein the VH and the VL are linked or conjugated from a C-terminus of the VL to an N-terminus of the VH.

TABLE 6

| Molecule | | | |
|---|---|---|---|
| ID | scFv VH | scFv Linker | scFv VL |
| ARB1 | EVQLVESGGGLVQPGGSLRLSCAASAFT | GGGGSGGGGSG | DIVMTQSPLSLPVTPGEPASISCRSSQSLVHGSG |
| | FSAHSMSWVRQAPGKGLELVASISPSGS | GGGSGGGGS | YTFLHWYLQKPGQSPQLLIYDAVTRATGVPDR |
| | VTYYPDSVKGRFTISRDNAKNSLYLQMN | (SEQ ID NO: 4) | FSGSGSGTDFTLKISRVEAEDVGVYYCMQTTE |
| | SLRAEDTAVYYCARDSGSYRDAFDIWG | | FPYTFGGGTKVEIK (SEQ ID NO: 18) |
| | QGTMVTVSS (SEQ ID NO: 54) | | |
| ARB2 | EVQLVESGGGLVQPGGSLRLSCAASGFT | GGGGSGGGGSG | DIVMTQSPLSLPVTPGEPASISCRSSQSLLHSTG |
| | FAGHAMSWVRQAPGKGLELVASISPSGS | GGGSGGGGS | YNFLHWYLQKPGQSPQLLIYDASKRAPGVPDR |
| | TTYYPDSVKGRFTISRDNAKNSLYLQMN | (SEQ ID NO: 4) | FSGSGSGTDFTLKISRVEAEDVGVYYCMQTVE |
| | SLRAEDTAVYYCARDGDSSDAFDIWGQ | | LPFTFGGGTKVEIK (SEQ ID NO: 19) |
| | GTMVTVSS (SEQ ID NO: 55) | | |
| ARB3 | QVQLQESGPGLVKPSQTLSLTCTVSGASI | GGGGSGGGGSG | EIVMTQSPATLSLSPGERATLSCRASQSVDRHL |
| | STIDYSWSWIRQPPGKGLEWIGYIDESGR | GGGSGGGGS | AWYQQKPGQAPRLLIYDVSNRAPGIPARFSGS |
| | IDYNPSLKSRVTMSVDTSKNQFSLKVNS | (SEQ ID NO: 4) | GSGTDFTLTISSLEPEDFAVYYCQQYGWPSITF |
| | VTAADTAVYYCAREGQWGQFDYWGQG | | GGGTKVEIK (SEQ ID NO: 20) |
| | TLVTVSS (SEQ ID NO: 56) | | |
| ARB4 | EVQLVESGGGLVQPGGSLRLSCAASGFT | GGGGSGGGGSG | DIVMTQSPLSLPVTPGEPASISCRSSQSLLSSYG |
| | FSNHAMSWVRQAPGKGLELVASINPSGS | GGGSGGGGS | YHNLHWYLQKPGQSPQLLIYDAYVRATGVPD |
| | VTYYPDSVKGRFTISRDNAKNSLYLQMN | (SEQ ID NO: 4) | RFSGSGSGTDFTLKISRVEAEDVGVYYCMQSK |
| | SLRAEDTAVYYCARDGDYGDYLDYWG | | ELPYTFGGGTKVEIK (SEQ ID NO: 21) |
| | QGTLVTVSS (SEQ ID NO: 57) | | |
| ARB5 | QVQLVQSGAEVKKPGASVKVSCKVSGD | GGGGSGGGGSG | DIQMTQSPSSVSASVGDRVTITCRASQDIGSNL |
| | TFTSNAIHWVRQAPGKGLEWMGGIVAG | GGGSGGGGS | AWYQQKPGKAPKLLIYSGSTLQSGVPSRFSGS |
| | SGHTIYAQKFQGRVTMTEDTSTDTAYME | (SEQ ID NO: 4) | GSGTDFTLTISSLQPEDFANYYCQQTSSSPPYTF |
| | LSSLKSEDTAVYYCAREGAEYNGFDPW | | GGGTKVEIK (SEQ ID NO: 22) |
| | GQGTLVTVSS (SEQ ID NO: 58) | | |
| ARB6 | QVQLVQSGAEVKKPGASVKVSCKVSGF | GGGGSGGGGSG | DIQMTQSPSSVSASVGDRVTITCRASQNIGNWL |
| | TFTSSVIHWVRQAPGKGLEWMGGISPRG | GGGSGGGGS | AWYQQKPGKAPKLLIYAASNLQRGVPSRFSGS |
| | ESTIYAQKFQGRVTMTEDTSTDTAYMEL | (SEQ ID NO: 4) | GSGTDFTLTISSLQPEDFANYYCQQYHNIPITFG |
| | SSLKSEDTAVYYCAREGASTGAFDIWGQ | | GGTKVEIK (SEQ ID NO: 23) |
| | GTMVTVSS (SEQ ID NO: 59) | | |
| ARB7 | QVQLVQSGAEVKKPGASVKVSCKVSGY | GGGGSGGGGSG | DIQMTQSPSSVSASVGDRVTITCRASQGIGTYL |
| | TLTGNAIHWVRQAPGKGLEWMGGIIPSI | GGGSGGGGS | AWYQQKPGKAPKLLIYDASILGSGVPSRFSGS |
| | GTAIYAQKFQGRVTMTEDTSTDTAYMEL | (SEQ ID NO: 4) | GSGTDFTLTISSLQPEDFANYYCQHYGTSPPTF |
| | SSLKSEDTAVYYCAKDRSDIGDIFDYWG | | GGGTKVEIK (SEQ ID NO: 24) |
| | QGTLVTVSS (SEQ ID NO: 60) | | |
| ARB8 | EVQLVESGGGLVQPGGSLRLSCAASGFT | GGGGSGGGGSG | DIVMTQSPLSLPVTPGEPASISCRSSQSLLHSTG |
| | FTTHSMSWVRQAPGKGLELVASINPAGSI | GGGSGGGGS | YNFLHWYLQKPGQSPQLLIYDTFHRATGVPDR |
| | TYYPDSVKGRFTISRDNAKNSLYLQMNS | (SEQ ID NO: 4) | FSGSGSGTDFTLKISRVEAEDVGVYYCMQSLQ |
| | LRAEDTAVYYCARDNNYGYGDHFDYW | | PRFTFGGGTKVEIK (SEQ ID NO: 25) |
| | GQGTLVTVSS (SEQ ID NO: 61) | | |
| ARB9 | EVQLVESGGGLVQPGGSLRLSCAASGFT | GGGGSGGGGSG | DIVMTQSPLSLPVTPGEPASISCRSSQSLLHSTG |
| | FGDHVMSWVRQAPGKGLELVASISPSGD | GGGSGGGGS | YNYLHWYLQKPGQSPQLLIYDTSTRASGVPDR |
| | VTYYPDSVKGRFTISRDNAKNSLYLQMN | (SEQ ID NO: 4) | FSGSGSGTDFTLKISRVEAEDVGVYYCMQTFH |
| | SLRAEDTAVYYCTTDGDSDAFDIWGQGT | | LPFTFGGGTKVEIK (SEQ ID NO: 26) |
| | MVTVSS (SEQ ID NO: 62) | | |
| ARB10 | EVQLVESGGGLVQPGGSLRLSCAASGLT | GGGGSGGGGSG | DIVMTQSPLSLPVTPGEPASISCRSSQSLLHSSG |
| | FSNHAMSWVRQAPGKGLELVASINPSGS | GGGSGGGGS | YNYLHWYLQKPGQSPQLLIYDTTNRATGVPD |
| | VTYYPDSVKGRFTISRDNAKNSLYLQMN | (SEQ ID NO: 4) | RFSGSGSGTDFTLKISRVEAEDVGVYYCMQTT |
| | SLRAEDTAVYYCTADDYGDYLDYWGQ | | QLPYTFGGGTKVEIK (SEQ ID NO: 27) |
| | GTLVTVSS (SEQ ID NO: 63) | | |
| ARB11 | QVQLVQSGAEVKKPGASVKVSCKVSGY | GGGGSGGGGSG | DIQMTQSPSSVSASVGDRVTITCRASQGIGRSL |
| | TFSNYVIHWVRQAPGKGLEWMGGIVAG | GGGSGGGGS | AWYQQKPGKAPKLLIYKDTERASGVPSRFSGS |
| | SGHTIYAQKFQGRVTMTEDTSTDTAYME | (SEQ ID NO: 4) | GSGTDFTLTISSLQPEDFANYYCQQVHTFPPTF |
| | LSSLKSEDTAVYYCATESAAGNWFDPW | | GGGTKVEIK (SEQ ID NO: 28) |
| | GQGTLVTVSS (SEQ ID NO: 64) | | |
| ARB12 | EVQLVESGGGLVQPGGSLRLSCAASGFT | GGGGSGGGGSG | DIVMTQSPLSLPVTPGEPASISCRSSQSLLHVTG |
| | FSDHTMSWVRQAPGKGLELVASINPSGS | GGGSGGGGS | YNYLHWYLQKPGQSPQLLIYETSKRAPGVPDR |
| | VTYYPDSVKGRFTISRDNAKNSLYLQMN | (SEQ ID NO: 4) | FSGSGSGTDFTLKISRVEAEDVGVYYCMQTTH |
| | SLRAEDTAVYYCARDGGYGDYFDYWG | | WPSTFGGGTKVEIK (SEQ ID NO: 29) |
| | QGTLVTVSS (SEQ ID NO: 65) | | |
| ARB13 | QVQLVQSGAEVKKPGASVKVSCKVSGT | GGGGSGGGGSG | DIQMTQSPSSVSASVGDRVTITCRASQSIGTNL |
| | PLPATIHWVRQAPGKGLEWMGGINPSGA | GGGSGGGGS | AWYQQKPGKAPKLLIYDASKRPTGVPSRFSGS |
| | TIYAQKFQGRVTMTEDTSTDTAYMELSS | (SEQ ID NO: 4) | GSGTDFTLTISSLQPEDFANYYCQQGYDIPLTF |
| | LKSEDTAVYYCAKDSDVAAAGSFFDYW | | GGGTKVEIK (SEQ ID NO: 30) |
| | GQGTLVTVSS (SEQ ID NO: 66) | | |
| ARB14 | QVQLVQSGAEVKKPGASVKVSCKVSGY | GGGGSGGGGSG | DIQMTQSPSSVSASVGDRVTITCRASQNIGDRL |
| | TFRNYAIHWVRQAPGKGLEWMGGISPSG | GGGSGGGGS | AWYQQKPGKAPKLLIYQDRNRPSGVPSRFSGS |
| | STTIYAQKFQGRVTMTEDTSTDTAYMEL | (SEQ ID NO: 4) | GSGTDFTLTISSLQPEDFANYYCQQYATSPFTF |
| | SSLKSEDTAVYYCARESAENYGDYLDY | | GGGTKVEIK (SEQ ID NO: 31) |
| | WGQGTLVTVSS (SEQ ID NO: 67) | | |
| ARB15 | QVQLVQSGAEVKKPGASVKVSCKVSGID | GGGGSGGGGSG | DIQMTQSPSSVSASVGDRVTITCRASQNIDTYL |
| | LTTSAIHWVRQAPGKGLEWMGGIAIGSG | GGGSGGGGS | AWYQQKPGKAPKLLIYEGTKRPSGVPSRFSGS |
| | HTIYAQKFQGRVTMTEDTSTDTAYMELS | (SEQ ID NO: 4) | GSGTDFTLTISSLQPEDFANYYCQQWDNLPLTF |
| | SLKSEDTAVYYCARDGNFGDYIEYWGQ | | GGGTKVEIK (SEQ ID NO: 32) |
| | GTLVTVSS (SEQ ID NO: 68) | | |
| ARB16 | QVQLVQSGAEVKKPGASVKVSCKVSGH | GGGGSGGGGSG | DIQMTQSPSSVSASVGDRVTITCRASESISSHLA |
| | TFTGYFIHWVRQAPGKGLEWMGGMDPI | GGGSGGGGS | WYQQKPGKAPKLLIYAGSSRATGVPSRFSGSG |
| | SGATIYAQKFQGRVTMTEDTSTDTAYME | (SEQ ID NO: 4) | SGTDFTLTISSLQPEDFANYYCHQYDTLPFTFG |
| | LSSLKSEDTAVYYCAREGTTIDAFDIWG | | GGTKVEIK (SEQ ID NO: 33) |
| | QGTMVTVSS (SEQ ID NO: 69) | | |
| ARB17 | EVQLVESGGGLVQPGGSLRLSCAASGLM | GGGGSGGGGSG | DIVMTQSPLSLPVTPGEPASISCRSSRSLVHGSG |
| | FSTSTMSWVRQAPGKGLELVASINPSGS | GGGSGGGGS | DNYLHWYLQKPGQSPQLLIYMTSNRAPGVPD |
| | VTYYPDSVKGRFTISRDNAKNSLYLQMN | (SEQ ID NO: 4) | RFSGSGSGTDFTLKISRVEAEDVGVYYCMQSG |
| | SLRAEDTAVYYCAREDGDSRGEAFDIWG | | HWPPTFGGGTKVEIK (SEQ ID NO: 34) |
| | QGTMVTVSS (SEQ ID NO: 70) | | |
| ARB18 | QVQLVQSGAEVKKPGASVKVSCKVSGID | GGGGSGGGGSG | DIQMTQSPSSVSASVGDRVTITCRASQNIDTWL |
| | LTTSAIHWVRQAPGKGLEWMGGINPGT | GGGSGGGGS | AWYQQKPGKAPKLLIYKASTLASGVPSRFSGS |
| | GSTIYAQKFQGRVTMTEDTSTDTAYMEL | (SEQ ID NO: 4) | GSGTDFTLTISSLQPEDFANYYCQQYNEVPLTF |
| | SSLKSEDTAVYYCAKEVAATGAYYYWG | | GGGTKVEIK (SEQ ID NO: 35) |
| | QGTLVTVSS (SEQ ID NO: 71) | | |
| ARB19 | EVQLVESGGGLVQPGGSLRLSCAASGFP | GGGGSGGGGSG | DIVMTQSPLSLPVTPGEPASISCRSSQSLLHSTG |
| | FSDYVMSWVRQAPGKGLELVASISPSGS | GGGSGGGGS | YNYLHWYLQKPGQSPQLLIYDATNRASGVPD |
| | TTYYPDSVKGRFTISRDNAKNSLYLQMN | (SEQ ID NO: 4) | RFSGSGSGTDFTLKISRVEAEDVGVYYCQQYA |
| | SLRAEDTAVYYCVRSGEWTDAFDIWGQ | | ASPPTFGGGTKVEIK (SEQ ID NO: 36) |
| | GTLVTVSS (SEQ ID NO: 72) | | |
| ARB20 | QVQLVQSGAEVKKPGASVKVSCKVSGY | GGGGSGGGGSG | DIQMTQSPSSVSASVGDRVTITCRASQDISTYL |
| | TFSDYVIHWVRQAPGKGLEWMGGINPY | GGGSGGGGS | AWYQQKPGKAPKLLIYDTSNRATGIPSRFSGS |
| | SGHTIYAQKFQGRVTMTEDTSTDTAYME | (SEQ ID NO: 4) | GSGTDFTLTISSLQPEDFANYYCQQSAKIPFTFG |
| | LSSLKSEDTAVYYCAKELDTAGNAFDIW | | GGTKVEIK (SEQ ID NO: 37) |
| | GQGTMVTVSS (SEQ ID NO: 73) | | |
| ARB21 | QVQLQESGPGLVKPSQTLSLTCTVSGAS | GGGGSGGGGSG | EIVMTQSPATLSLSPGERATLSCRASHSVTSNL |
| | VRDHYWSWIRQPPGKGLEWIGYAHYSGI | GGGSGGGGS | AWYQQKPGQAPRLLIYGASSRVPGIPARFSGS |
| | TDYNPSLKSRVTMSVDTSKNQFSLKVNS | (SEQ ID NO: 4) | GSGTDFTLTISSLEPEDFAVYYCQQYDNRPITF |
| | VTAADTAVYYCAIYSSGWWEDYWGQG | | GGGTKVEIK (SEQ ID NO: 38) |
| | TLVTVSS (SEQ ID NO: 74) | | |
| ARB22 | QVQLVQSGAEVKKPGASVKVSCKVSGY | GGGGSGGGGSG | DIQMTQSPSSVSASVGDRVTITCRASQSIAPLA |
| | PFPSYDIHWVRQAPGKGLEWMGGMNPT | GGGSGGGGS | WYQQKPGKAPKLLIYAASTLQPGVPSRFSGSG |
| | TGDTIYAQKFQGRVTMTEDTSTDTAYME | (SEQ ID NO: 4) | SGTDFTLTISSLQPEDFANYYCLQYHVLPITFG |
| | LSSLKSEDTAVYYCARETGGGEMAFDIW | | GGTKVEIK (SEQ ID NO: 39) |
| | GQGTMVTVSS (SEQ ID NO: 75) | | |
| ARB23 | QVQLQESGPGLVKPSQTLSLTCTVSEYAI | GGGGSGGGGSG | EIVMTQSPATLSLSPGERATLSCRASQNIGTNL |
| | RSDYTWSWIRQPPGKGLEWIGYIHHSGL | GGGSGGGGS | AWYQQKPGQAPRLLIYDASKRPTGIPARFSGS |
| | TDYNPSLKSRVTMSVDTSKNQFSLKVNS | (SEQ ID NO: 4) | GSGTDFTLTISSLEPEDFAVYYCQQYGTTPFTF |
| | VTAADTAVYYCARVRYSSSSEGWFDPW | | GGGTKVEIK (SEQ ID NO: 40) |
| | GQGTLVTVSS (SEQ ID NO: 76) | | |
| ARB24 | QVQLQESGPGLVKPSQTLSLTCTVSGASI | GGGGGGGGSG | EIVMTQSPATLSLSPGERATLSCRASESIGSNLA |
| | STSDTWSWIRQPPGKGLEWIGYIHHSGIT | GGGSGGGGS | WYQQKPGQAPRLLIYDASNRATGIPARFSGSG |
| | DYNPSLKSRVTMSVDTSKNQFSLKVNSV | (SEQ ID NO: 4) | SGTDFTLTISSLEPEDFAVYYCQQYDHWPLTFG |
| | TAADTAVYYCARGGSSGNWYLLDYWG | | GGTKVEIK (SEQ ID NO: 41) |
| | QGTLVTVSS (SEQ ID NO: 77) | | |
| ARB25 | EVQLVESGGGLVQPGGSLRLSCAASRFT | GGGGSGGGGSG | DIVMTQSPLSLPVTPGEPASISCRSSRSLVHGSG |
| | FSDYHMSWVRQAPGKGLELVASIDTEGK | GGGSGGGGS | DNYLHWYLQKPGQSPQLLIYMASNRAPGVPD |
| | TYYPDSVKGRFTISRDNAKNSLYLQMNS | (SEQ ID NO: 4) | RFSGSGSGTDFTLKISRVEAEDVGVYYCMQAL |
| | LRAEDTAVYYCARDVGDWYFDLWGRG | | RAPFSFGGGTKVEIK (SEQ ID NO: 42) |
| | TLVTVSS (SEQ ID NO: 78) | | |
| ARB26 | QVQLQESGPGLVKPSQTLSLTCTVSGVSL | GGGGSGGGGSG | EIVMTQSPATLSLSPGERATLSCRASQSLSSSHL |
| | SNARMGWSWIRQPPGKGLEWIGYVHTS | GGGSGGGGS | AWYQQKPGQAPRLLIYDASIRVPGIPARFSGSG |
| | GSTDYNPSLKSRVTMSVDTSKNQFSLKV | (SEQ ID NO: 4) | SGTDFTLTISSLEPEDFAVYYCQQYAVPPITFG |
| | NSVTAADTAVYYCARDAGNWFDPWGQ | | GGTKVEIK (SEQ ID NO: 43) |
| | GTLVTVSS (SEQ ID NO: 79) | | |
| ARB27 | EVQLVESGGGLVQPGRSLRLSCAASGST | GGGGSGGGGSG | DIQMTQSPSSLSASVGDRVTITCRASQGIGSYL |
| | LSSYGMHWVRQAPGKGLEWVSAISYDA | GGGSGGGGS | AWYQQKPGKAPKLLIYEASRLESGVPSRFSGS |
| | STIDYADSVEGRFTISRDNAKNSLYLQM | (SEQ ID NO: 4) | GSGTDFTLTISSLQPEDVATYYCQQGYNAPITF |
| | NSLRAEDTAVYYCARDLLGYGMDVWG | | GGGTKVEIK (SEQ ID NO: 44) |
| | QGTMVTVSS (SEQ ID NO: 80) | | |
| ARB28 | QVQLVQSGAEVKKPGASVKVSCKVSGS | GGGGSGGGGSG | DIQMTQSPSSVSASVGDRVTITCRASQDIGNWL |
| | AFTSNDIHWVRQAPGKGLEWMGGINPRS | GGGSGGGGS | AWYQQKPGKAPKLLIYDASTLDTGVPSRFSGS |
| | GATIYAQKFQGRVTMTEDTSTDTAYMEL | (SEQ ID NO: 4) | GSGTDFTLTISSLQPEDFANYYCLQDYSYPLTF |
| | SSLKSEDTAVYYCARALSDDSSGYDAFD | | GGGTKVEIK (SEQ ID NO: 45) |
| | IWGQGTMVTVSS (SEQ ID NO: 81) | | |
| ARB29 | EVQLVESGGGLVQPGRSLRLSCAASGST | GGGGSGGGGSG | DIQMTQSPSSLSASVGDRVTITCRASQDISNWL |
| | LSSYGMHWVRQAPGKGLEWVSAISYDA | GGGSGGGGS | AWYQQKPGKAPKLLIYEASRLESGVPSRFSGS |
| | STIDYADSVEGRFTISRDNAKNSLYLQM | (SEQ ID NO: 4) | GSGTDFTLTISSLQPEDVATYYCQQGYNAPITF |
| | NSLRAEDTAVYYCARDLLGYGMDVWG | | GGGTKVEIK (SEQ ID NO: 46) |
| | QGTMVTVSS (SEQ ID NO: 80) | | |
| ARB30 | EVQLVESGGGLVQPGRSLRLSCAASGEN | GGGGSGGGGSG | DIQMTQSPSSLSASVGDRVTITCRASQDISNWL |
| | FGAFAMHWVRQAPGKGLEWVSAINQGG | GGGSGGGGS | AWYQQKPGKAPKLLIYDASSLVSGVPSRFSGS |
| | SEVDYADSVEGRFTISRDNAKNSLYLQM | (SEQ ID NO: 4) | GSGTDFTLTISSLQPEDVATYYCHQIYDTPPTF |
| | NSLRAEDTAVYYCARDPGLPVSAFDIWG | | GGGTKVEIK (SEQ ID NO: 47) |
| | LGTMVTVSS (SEQ ID NO: 82) | | |
| ARB31 | EVQLVESGGGLVQPGRSLRLSCAASGFT | GGGGSGGGGSG | DIQMTQSPSSLSASVGDRVTITCRASQAISGSL |
| | FENYGMHWVRQAPGKGLEWVSAISYDA | GGGSGGGGS | AWYQQKPGKAPKLLIYATSRLESGVPSRFSGS |
| | STIDYADSVEGRFTISRDNAKNSLYLQM | (SEQ ID NO: 4) | GSGTDFTLTISSLQPEDVATYYCQQSGSTPITFG |
| | NSLRAEDTAVYYCASEYGLAFDQWGQG | | GGTKVEIK (SEQ ID NO: 48) |
| | TLVTVSS (SEQ ID NO: 83) | | |
| ARB32 | EVQLVESGGGLVQPGRSLRLSCAASGST | GGGGSGGGGSG | DIQMTQSPSSLSASVGDRVTITCRASQGIGSYL |
| | LSSYGMHWVRQAPGKGLEWVSAISYDA | GGGSGGGGS | AWYQQKPGKAPKLLIYEASRLESGVPSRFSGS |
| | STIDYADSVEGRFTISRDNAKNSLYLQM | (SEQ ID NO: 4) | GSGTDFTLTISSLQPEDVATYYCQQGYNAPITF |
| | NSLRAEDTAVYYCASEYGLAFDQWGQG | | GGGTKVEIK (SEQ ID NO: 44) |
| | TLVTVSS (SEQ ID NO: 84) | | |
| ARB33 | EVQLVESGGGLVQPGRSLRLSCAASGLT | GGGGSGGGGSG | DIQMTQSPSSLSASVGDRVTITCRASQDIAGWL |
| | FSNHGMHWVRQAPGKGLEWVSAISSSA | GGGSGGGGS | AWYQQKPGKAPKLLIYDASTLQGGVPSRFSGS |
| | DIVDYADSVEGRFTISRDNAKNSLYLQM | (SEQ ID NO: 4) | GSGTDFTLTISSLQPEDVATYYCQQSYTAPLNF |
| | NSLRAEDTAVYYCASEGVPYGMDVWG | | GGGTKVEIK (SEQ ID NO: 49) |
| | QGTMVTVSS (SEQ ID NO: 85) | | |
| ARB34 | EVQLVESGGGLVQPGRSLRLSCAASGST | GGGGSGGGGSG | DIQMTQSPSSLSASVGDRVTITCRASQGIGSYL |
| | LSSYGMHWVRQAPGKGLEWVSAISYDA | GGGSGGGGS | AWYQQKPGKAPKLLIYEASRLESGVPSRFSGS |
| | STIDYADSVEGRFTISRDNAKNSLYLQM | (SEQ ID NO: 4) | GSGTDFTLTISSLQPEDVATYYCQQGYNAPITF |
| | NSLRAEDTAVYYCASEGVPYGMDVWG | | GGGTKVEIK (SEQ ID NO: 44) |
| | QGTMVTVSS (SEQ ID NO: 86) | | |
| ARB35 | QVQLQESGPGLVKPSQTLSLTCTVSGESF | GGGGSGGGGSG | EIVMTQSPATLSLSPGERATLSCRASQTIGTNL |
| | SDFYWSWIRQPPGKGLEWIGYIDHTGST | GGGSGGGGS | AWYQQKPGQAPRLLIYDASTRANGIPARFSGS |
| | DYNPSLKSRVTMSVDTSKNQFSLKVNSV | (SEQ ID NO: 4) | GSGTDFTLTISSLEPEDFAVYYCQQYAAPPLTF |
| | TAADTAVYYCAGDKYADGFDVWGQGT | | GGGTKVEIK (SEQ ID NO: 50) |
| | MVTVSS (SEQ ID NO: 87) | | |
| ARB36 | QVQLQESGPGLVKPSQTLSLTCTVSGFSL | GGGGSGGGGSG | EIVMTQSPATLSLSPGERATLSCRASQSIRSDLA |
| | STSGVRWSWIRQPPGKGLEWIGYINPSGT | GGGSGGGGS | WYQQKPGQAPRLLIYDASHRPAGIPARFSGSG |
| | TDYNPSLKSRVTMSVDTSKNQFSLKVNS | (SEQ ID NO: 4) | SGTDFTLTISSLEPEDFAVYYCQQYGSVPRPTF |
| | VTAADTAVYYCARGGGYCSGGSCYDW | | GGGTKVEIK (SEQ ID NO: 51) |
| | YFDLWGRGTLVTVSS | | |
| | (SEQ ID NO: 88) | | |
| ARB37 | QVQLQESGPGLVKPSQTLSLTCTVSGFSL | GGGGSGGGGSG | EIVMTQSPATLSLSPGERATLSCRASQSIRSDLA |
| | STSGVRWSWIRQPPGKGLEWIGYINPSGT | GGGSGGGGS | WYQQKPGQAPRLLIYDATSRAAGIPARFSGSG |
| | TDYNPSLKSRVTMSVDTSKNQFSLKVNS | (SEQ ID NO: 4) | SGTDFTLTISSLEPEDFAVYYCQEYGSAPLTFG |
| | VTAADTAVYYCARGGGYCSGGSCYDW | | GGTKVEIK (SEQ ID NO: 52) |
| | YFDLWGRGTLVTVSS | | |
| | (SEQ ID NO: 88) | | |
| ARB38 | QVQLQESGPGLVKPSQTLSLTCTVSGESF | GGGGSGGGGSG | EIVMTQSPATLSLSPGERATLSCRASMGVSSNL |
| | SGYSWSWIRQPPGKGLEWIGYITHSGTID | GGGSGGGGS | AWYQQKPGQAPRLLIYDASNRAAGIPARFSGS |
| | YNPSLKSRVTMSVDTSKNQFSLKVNSVT | (SEQ ID NO: 4) | GSGTDFTLTISSLEPEDFAVYYCQQYAEGPLTF |
| | AADTAVYYCAKPLDFGDYKDAFDIWGQ | | GGGTKVEIK (SEQ ID NO: 53) |
| | GTMVTVSS (SEQ ID NO: 89) | | |
| ARB39 | EVQLVESGGGLVQPGRSLRLSCAASGST | GGGGSGGGGSG | DIQMTQSPSSLSASVGDRVTITCRASQGIGSYL |
| | LSSYGMHWVRQAPGKGLEWVSAISYDA | GGGSGGGGS | AWYQQKPGKAPKLLIYEASRLESGVPSRFSGS |
| | STIDYADSVEGRFTISRDNAKNSLYLQM | (SEQ ID NO: 4) | GSGTDFTLTISSLQPEDVATYYCQQGYNAPITF |
| | NSLRAEDTAVYYCARDGISGIDYWGQGT | | GGGTKVEIK (SEQ ID NO: 44) |
| | LVTVSS (SEQ ID NO: 90) | | |

In some embodiments, the anti-FcγRIIβ antibody comprises a polypeptide comprising an amino acid sequence comprising any one variable heavy chains and any one variable light chains provided in Table 33 below. In some embodiments, the anti-FcγRIIβ antibody is an Fab format (IgG format) comprising an amino acid sequence having any one variable heavy chains and any one variable light chains provided in Table 3 below.

TABLE 33

| Molecule | | |
|---|---|---|
| ID | VH | VL |
| ARB1 | EVQLVESGGGLVQPGGSLRLSCAASAFTFSAHSMSWVR | DIVMTQSPLSLPVTPGEPASISCRSSQSLVHGSGYTFL |
| | QAPGKGLELVASISPSGSVTYYPDSVKGRFTISRDNAKN | HWYLQKPGQSPQLLIYDAVTRATGVPDRFSGSGSGT |
| | SLYLQMNSLRAEDTAVYYCARDSGSYRDAFDIWGQGT | DFTLKISRVEAEDVGVYYCMQTTEFPYTFGGGTKVEI |
| | MVTVSS (SEQ ID NO: 54) | K (SEQ ID NO: 18) |
| ARB2 | EVQLVESGGGLVQPGGSLRLSCAASGFTFAGHAMSWV | DIVMTQSPLSLPVTPGEPASISCRSSQSLLHSTGYNFLH |
| | RQAPGKGLELVASISPSGSTTYYPDSVKGRFTISRDNAK | WYLQKPGQSPQLLIYDASKRAPGVPDRFSGSGSGTDF |
| | NSLYLQMNSLRAEDTAVYYCARDGDSSDAFDIWGQGT | TLKISRVEAEDVGVYYCMQTVELPFTFGGGTKVEIK |
| | MVTVSS (SEQ ID NO: 55) | (SEQ ID NO: 19) |
| ARB3 | QVQLQESGPGLVKPSQTLSLTCTVSGASISTIDYSWSWI | EIVMTQSPATLSLSPGERATLSCRASQSVDRHLAWYQ |
| | RQPPGKGLEWIGYIDESGRIDYNPSLKSRVTMSVDTSKN | QKPGQAPRLLIYDVSNRAPGIPARFSGSGSGTDFTLTI |
| | QFSLKVNSVTAADTAVYYCAREGQWGQFDYWGQGTL | SSLEPEDFAVYYCQQYGWPSITFGGGTKVEIK (SEQ |
| | VTVSS (SEQ ID NO: 56) | ID NO: 20) |
| ARB4 | EVQLVESGGGLVQPGGSLRLSCAASGFTFSNHAMSWV | DIVMTQSPLSLPVTPGEPASISCRSSQSLLSSYGYHNL |
| | RQAPGKGLELVASINPSGSVTYYPDSVKGRFTISRDNAK | HWYLQKPGQSPQLLIYDAYVRATGVPDRFSGSGSGT |
| | NSLYLQMNSLRAEDTAVYYCARDGDYGDYLDYWGQG | DFTLKISRVEAEDVGVYYCMQSKELPYTFGGGTKVEI |
| | TLVTVSS (SEQ ID NO: 57) | K (SEQ ID NO: 21) |
| ARB5 | QVQLVQSGAEVKKPGASVKVSCKVSGDTFTSNAIHWV | DIQMTQSPSSVSASVGDRVTITCRASQDIGSNLAWYQ |
| | RQAPGKGLEWMGGIVAGSGHTIYAQKFQGRVTMTEDT | QKPGKAPKLLIYSGSTLQSGVPSRFSGSGSGTDFTLTIS |
| | STDTAYMELSSLKSEDTAVYYCAREGAEYNGFDPWGQ | SLQPEDFANYYCQQTSSSPPYTFGGGTKVEIK (SEQ ID |
| | GTLVTVSS (SEQ ID NO: 58) | NO: 22) |
| ARB6 | QVQLVQSGAEVKKPGASVKVSCKVSGFTFTSSVIHWVR | DIQMTQSPSSVSASVGDRVTITCRASQNIGNWLAWY |
| | QAPGKGLEWMGGISPRGESTIYAQKFQGRVTMTEDTST | QQKPGKAPKLLIYAASNLQRGVPSRFSGSGSGTDFTL |
| | DTAYMELSSLKSEDTAVYYCAREGASTGAFDIWGQGT | TISSLQPEDFANYYCQQYHNIPITFGGGTKVEIK (SEQ |
| | MVTVSS (SEQ ID NO: 59) | ID NO: 23) |
| ARB7 | QVQLVQSGAEVKKPGASVKVSCKVSGYTLTGNAIHWV | DIQMTQSPSSVSASVGDRVTITCRASQGIGTYLAWYQ |
| | RQAPGKGLEWMGGIIPSIGTAIYAQKFQGRVTMTEDTS | QKPGKAPKLLIYDASILGSGVPSRFSGSGSGTDFTLTIS |
| | TDTAYMELSSLKSEDTAVYYCAKDRSDIGDIFDYWGQ | SLQPEDFANYYCQHYGTSPPTFGGGTKVEIK (SEQ ID |
| | GTLVTVSS (SEQ ID NO: 60) | NO: 24) |
| ARB8 | EVQLVESGGGLVQPGGSLRLSCAASGFTFTTHSMSWVR | DIVMTQSPLSLPVTPGEPASISCRSSQSLLHSTGYNFLH |
| | QAPGKGLELVASINPAGSITYYPDSVKGRFTISRDNAKN | WYLQKPGQSPQLLIYDTFHRATGVPDRFSGSGSGTDF |
| | SLYLQMNSLRAEDTAVYYCARDNNYGYGDHFDYWGQ | TLKISRVEAEDVGVYYCMQSLQPRFTFGGGTKVEIK |
| | GTLVTVSS (SEQ ID NO: 61) | (SEQ ID NO: 25) |
| ARB9 | EVQLVESGGGLVQPGGSLRLSCAASGFTFGDHVMSWV | DIVMTQSPLSLPVTPGEPASISCRSSQSLLHSTGYNYL |
| | RQAPGKGLELVASISPSGDVTYYPDSVKGRFTISRDNAK | HWYLQKPGQSPQLLIYDTSTRASGVPDRFSGSGSGTD |
| | NSLYLQMNSLRAEDTAVYYCTTDGDSDAFDIWGQGTM | FTLKISRVEAEDVGVYYCMQTFHLPFTFGGGTKVEIK |
| | VTVSS (SEQ ID NO: 62) | (SEQ ID NO: 26) |
| ARB10 | EVQLVESGGGLVQPGGSLRLSCAASGLTFSNHAMSWV | DIVMTQSPLSLPVTPGEPASISCRSSQSLLHSSGYNYL |
| | RQAPGKGLELVASINPSGSVTYYPDSVKGRFTISRDNAK | HWYLQKPGQSPQLLIYDTTNRATGVPDRFSGSGSGT |
| | NSLYLQMNSLRAEDTAVYYCTADDYGDYLDYWGQGT | DFTLKISRVEAEDVGVYYCMQTTQLPYTFGGGTKVEI |
| | LVTVSS (SEQ ID NO: 63) | K (SEQ ID NO: 27) |
| ARB11 | QVQLVQSGAEVKKPGASVKVSCKVSGYTFSNYVIHWV | DIQMTQSPSSVSASVGDRVTITCRASQGIGRSLAWYQ |
| | RQAPGKGLEWMGGIVAGSGHTIYAQKFQGRVTMTEDT | QKPGKAPKLLIYKDTERASGVPSRFSGSGSGTDFTLTI |
| | STDTAYMELSSLKSEDTAVYYCATESAAGNWFDPWGQ | SSLQPEDFANYYCQQVHTFPPTFGGGTKVEIK (SEQ |
| | GTLVTVSS (SEQ ID NO: 64) | ID NO: 28) |
| ARB12 | EVQLVESGGGLVQPGGSLRLSCAASGFTFSDHTMSWVR | DIVMTQSPLSLPVTPGEPASISCRSSQSLLHVTGYNYL |
| | QAPGKGLELVASINPSGSVTYYPDSVKGRFTISRDNAKN | HWYLQKPGQSPQLLIYETSKRAPGVPDRFSGSGSGTD |
| | SLYLQMNSLRAEDTAVYYCARDGGYGDYFDYWGQGT | FTLKISRVEAEDVGVYYCMQTTHWPSTFGGGTKVEI |
| | LVTVSS (SEQ ID NO: 65) | K (SEQ ID NO: 29) |
| ARB13 | QVQLVQSGAEVKKPGASVKVSCKVSGTPLPATIHWVR | DIQMTQSPSSVSASVGDRVTITCRASQSIGTNLAWYQ |
| | QAPGKGLEWMGGINPSGATIYAQKFQGRVTMTEDTST | QKPGKAPKLLIYDASKRPTGVPSRFSGSGSGTDFTLTI |
| | DTAYMELSSLKSEDTAVYYCAKDSDVAAAGSFFDYWG | SSLQPEDFANYYCQQGYDIPLTFGGGTKVEIK (SEQ |
| | QGTLVTVSS (SEQ ID NO: 66) | ID NO: 30) |
| ARB14 | QVQLVQSGAEVKKPGASVKVSCKVSGYTFRNYAIHWV | DIQMTQSPSSVSASVGDRVTITCRASQNIGDRLAWYQ |
| | RQAPGKGLEWMGGISPSGSTTIYAQKFQGRVTMTEDTS | QKPGKAPKLLIYQDRNRPSGVPSRFSGSGSGTDFTLTI |
| | TDTAYMELSSLKSEDTAVYYCARESAENYGDYLDYWG | SSLQPEDFANYYCQQYATSPFTFGGGTKVEIK (SEQ |
| | QGTLVTVSS (SEQ ID NO: 67) | ID NO: 31) |
| ARB15 | QVQLVQSGAEVKKPGASVKVSCKVSGIDLTTSAIHWVR | DIQMTQSPSSVSASVGDRVTITCRASQNIDTYLAWYQ |
| | QAPGKGLEWMGGIAIGSGHTIYAQKFQGRVTMTEDTST | QKPGKAPKLLIYEGTKRPSGVPSRFSGSGSGTDFTLTI |
| | DTAYMELSSLKSEDTAVYYCARDGNFGDYIEYWGQGT | SSLQPEDFANYYCQQWDNLPLTFGGGTKVEIK (SEQ |
| | LVTVSS (SEQ ID NO: 68) | ID NO: 32) |
| ARB16 | QVQLVQSGAEVKKPGASVKVSCKVSGHTFTGYFIHWV | DIQMTQSPSSVSASVGDRVTITCRASESISSHLAWYQQ |
| | RQAPGKGLEWMGGMDPISGATIYAQKFQGRVTMTEDT | KPGKAPKLLIYAGSSRATGVPSRFSGSGSGTDFTLTIS |
| | STDTAYMELSSLKSEDTAVYYCAREGTTIDAFDIWGQG | SLQPEDFANYYCHQYDTLPFTFGGGTKVEIK (SEQ ID |
| | TMVTVSS (SEQ ID NO: 69) | NO: 33) |
| ARB17 | EVQLVESGGGLVQPGGSLRLSCAASGLMFSTSTMSWV | DIVMTQSPLSLPVTPGEPASISCRSSRSLVHGSGDNYL |
| | RQAPGKGLELVASINPSGSVTYYPDSVKGRFTISRDNAK | HWYLQKPGQSPQLLIYMTSNRAPGVPDRFSGSGSGT |
| | NSLYLQMNSLRAEDTAVYYCAREDGDSRGEAFDIWGQ | DFTLKISRVEAEDVGVYYCMQSGHWPPTFGGGTKVE |
| | GTMVTVSS (SEQ ID NO: 70) | IK (SEQ ID NO: 34) |
| ARB18 | QVQLVQSGAEVKKPGASVKVSCKVSGIDLTTSAIHWVR | DIQMTQSPSSVSASVGDRVTITCRASQNIDTWLAWYQ |
| | QAPGKGLEWMGGINPGTGSTIYAQKFQGRVTMTEDTS | QKPGKAPKLLIYKASTLASGVPSRFSGSGSGTDFTLTI |
| | TDTAYMELSSLKSEDTAVYYCAKEVAATGAYYYWGQ | SSLQPEDFANYYCQQYNEVPLTFGGGTKVEIK (SEQ |
| | GTLVTVSS (SEQ ID NO: 71) | ID NO: 35) |
| ARB19 | EVQLVESGGGLVQPGGSLRLSCAASGFPFSDYVMSWV | DIVMTQSPLSLPVTPGEPASISCRSSQSLLHSTGYNYL |
| | RQAPGKGLELVASISPSGSTTYYPDSVKGRFTISRDNAK | HWYLQKPGQSPQLLIYDATNRASGVPDRFSGSGSGT |
| | NSLYLQMNSLRAEDTAVYYCVRSGEWTDAFDIWGQGT | DFTLKISRVEAEDVGVYYCQQYAASPPTFGGGTKVEI |
| | LVTVSS (SEQ ID NO: 72) | K (SEQ ID NO: 36) |
| ARB20 | QVQLVQSGAEVKKPGASVKVSCKVSGYTFSDYVIHWV | DIQMTQSPSSVSASVGDRVTITCRASQDISTYLAWYQ |
| | RQAPGKGLEWMGGINPYSGHTIYAQKFQGRVTMTEDT | QKPGKAPKLLIYDTSNRATGIPSRFSGSGSGTDFTLTIS |
| | STDTAYMELSSLKSEDTAVYYCAKELDTAGNAFDIWG | SLQPEDFANYYCQQSAKIPFTFGGGTKVEIK (SEQ ID |
| | QGTMVTVSS (SEQ ID NO: 73) | NO: 37) |
| ARB21 | QVQLQESGPGLVKPSQTLSLTCTVSGASVRDHYWSWIR | EIVMTQSPATLSLSPGERATLSCRASHSVTSNLAWYQ |
| | QPPGKGLEWIGYAHYSGITDYNPSLKSRVTMSVDTSKN | QKPGQAPRLLIYGASSRVPGIPARFSGSGSGTDFTLTIS |
| | QFSLKVNSVTAADTAVYYCAIYSSGWWEDYWGQGTL | SLEPEDFAVYYCQQYDNRPITFGGGTKVEIK (SEQ ID |
| | VTVSS (SEQ ID NO: 74) | NO: 38) |
| ARB22 | QVQLVQSGAEVKKPGASVKVSCKVSGYPFPSYDIHWV | DIQMTQSPSSVSASVGDRVTITCRASQSIAPLAWYQQ |
| | RQAPGKGLEWMGGMNPTTGDTIYAQKFQGRVTMTED | KPGKAPKLLIYAASTLQPGVPSRFSGSGSGTDFTLTISS |
| | TSTDTAYMELSSLKSEDTAVYYCARETGGGEMAFDIW | LQPEDFANYYCLQYHVLPITFGGGTKVEIK (SEQ ID |
| | GQGTMVTVSS (SEQ ID NO: 75) | NO: 39) |
| ARB23 | QVQLQESGPGLVKPSQTLSLTCTVSEYAIRSDYTWSWIR | EIVMTQSPATLSLSPGERATLSCRASQNIGTNLAWYQ |
| | QPPGKGLEWIGYIHHSGLTDYNPSLKSRVTMSVDTSKN | QKPGQAPRLLIYDASKRPTGIPARFSGSGSGTDFTLTIS |
| | QFSLKVNSVTAADTAVYYCARVRYSSSSEGWFDPWGQ | SLEPEDFAVYYCQQYGTTPFTFGGGTKVEIK (SEQ ID |
| | GTLVTVSS (SEQ ID NO: 76) | NO: 40) |
| ARB24 | QVQLQESGPGLVKPSQTLSLTCTVSGASISTSDTWSWIR | EIVMTQSPATLSLSPGERATLSCRASESIGSNLAWYQQ |
| | QPPGKGLEWIGYIHHSGITDYNPSLKSRVTMSVDTSKN | KPGQAPRLLIYDASNRATGIPARFSGSGSGTDFTLTISS |
| | QFSLKVNSVTAADTAVYYCARGGSSGNWYLLDYWGQ | LEPEDFAVYYCQQYDHWPLTFGGGTKVEIK (SEQ ID |
| | GTLVTVSS (SEQ ID NO: 77) | NO: 41) |
| ARB25 | EVQLVESGGGLVQPGGSLRLSCAASRFTFSDYHMSWV | DIVMTQSPLSLPVTPGEPASISCRSSRSLVHGSGDNYL |
| | RQAPGKGLELVASIDTEGKTYYPDSVKGRFTISRDNAK | HWYLQKPGQSPQLLIYMASNRAPGVPDRFSGSGSGT |
| | NSLYLQMNSLRAEDTAVYYCARDVGDWYFDLWGRGT | DFTLKISRVEAEDVGVYYCMQALRAPFSFGGGTKVEI |
| | LVTVSS (SEQ ID NO: 78) | K (SEQ ID NO: 42) |
| ARB26 | QVQLQESGPGLVKPSQTLSLTCTVSGVSLSNARMGWS | EIVMTQSPATLSLSPGERATLSCRASQSLSSSHLAWY |
| | WIRQPPGKGLEWIGYVHTSGSTDYNPSLKSRVTMSVDT | QQKPGQAPRLLIYDASIRVPGIPARFSGSGSGTDFTLTI |
| | SKNQFSLKVNSVTAADTAVYYCARDAGNWFDPWGQG | SSLEPEDFAVYYCQQYAVPPITFGGGTKVEIK (SEQ ID |
| | TLVTVSS (SEQ ID NO: 79) | NO: 43) |
| ARB27 | EVQLVESGGGLVQPGRSLRLSCAASGSTLSSYGMHWV | DIQMTQSPSSLSASVGDRVTITCRASQGIGSYLAWYQ |
| | RQAPGKGLEWVSAISYDASTIDYADSVEGRFTISRDNA | QKPGKAPKLLIYEASRLESGVPSRFSGSGSGTDFTLTIS |
| | KNSLYLQMNSLRAEDTAVYYCARDLLGYGMDVWGQG | SLQPEDVATYYCQQGYNAPITFGGGTKVEIK (SEQ ID |
| | TMVTVSS (SEQ ID NO: 80) | NO: 44) |
| ARB28 | QVQLVQSGAEVKKPGASVKVSCKVSGSAFTSNDIHWV | DIQMTQSPSSVSASVGDRVTITCRASQDIGNWLAWY |
| | RQAPGKGLEWMGGINPRSGATIYAQKFQGRVTMTEDT | QQKPGKAPKLLIYDASTLDTGVPSRFSGSGSGTDFTL |
| | STDTAYMELSSLKSEDTAVYYCARALSDDSSGYDAFDI | TISSLQPEDFANYYCLQDYSYPLTFGGGTKVEIK (SEQ |
| | WGQGTMVTVSS (SEQ ID NO: 81) | ID NO: 45) |
| ARB29 | EVQLVESGGGLVQPGRSLRLSCAASGSTLSSYGMHWV | DIQMTQSPSSLSASVGDRVTITCRASQDISNWLAWYQ |
| | RQAPGKGLEWVSAISYDASTIDYADSVEGRFTISRDNA | QKPGKAPKLLIYEASRLESGVPSRFSGSGSGTDFTLTIS |
| | KNSLYLQMNSLRAEDTAVYYCARDLLGYGMDVWGQG | SLQPEDVATYYCQQGYNAPITFGGGTKVEIK (SEQ ID |
| | TMVTVSS (SEQ ID NO: 80) | NO: 46) |
| ARB30 | EVQLVESGGGLVQPGRSLRLSCAASGFNFGAFAMHWV | DIQMTQSPSSLSASVGDRVTITCRASQDISNWLAWYQ |
| | RQAPGKGLEWVSAINQGGSEVDYADSVEGRFTISRDNA | QKPGKAPKLLIYDASSLVSGVPSRFSGSGSGTDFTLTI |
| | KNSLYLQMNSLRAEDTAVYYCARDPGLPVSAFDIWGL | SSLQPEDVATYYCHQIYDTPPTFGGGTKVEIK (SEQ ID |
| | GTMVTVSS (SEQ ID NO: 82) | NO: 47) |
| ARB31 | EVQLVESGGGLVQPGRSLRLSCAASGFTFENYGMHWV | DIQMTQSPSSLSASVGDRVTITCRASQAISGSLAWYQ |
| | RQAPGKGLEWVSAISYDASTIDYADSVEGRFTISRDNA | QKPGKAPKLLIYATSRLESGVPSRFSGSGSGTDFTLTIS |
| | KNSLYLQMNSLRAEDTAVYYCASEYGLAFDQWGQGT | SLQPEDVATYYCQQSGSTPITFGGGTKVEIK (SEQ ID |
| | LVTVSS (SEQ ID NO: 83) | NO: 48) |
| ARB32 | EVQLVESGGGLVQPGRSLRLSCAASGSTLSSYGMHWV | DIQMTQSPSSLSASVGDRVTITCRASQGIGSYLAWYQ |
| | RQAPGKGLEWVSAISYDASTIDYADSVEGRFTISRDNA | QKPGKAPKLLIYEASRLESGVPSRFSGSGSGTDFTLTIS |
| | KNSLYLQMNSLRAEDTAVYYCASEYGLAFDQWGQGT | SLQPEDVATYYCQQGYNAPITFGGGTKVEIK (SEQ ID |
| | LVTVSS (SEQ ID NO: 84) | NO: 44) |
| ARB33 | EVQLVESGGGLVQPGRSLRLSCAASGLTFSNHGMHWV | DIQMTQSPSSLSASVGDRVTITCRASQDIAGWLAWYQ |
| | RQAPGKGLEWVSAISSSADIVDYADSVEGRFTISRDNAK | QKPGKAPKLLIYDASTLQGGVPSRFSGSGSGTDFTLTI |
| | NSLYLQMNSLRAEDTAVYYCASEGVPYGMDVWGQGT | SSLQPEDVATYYCQQSYTAPLNFGGGTKVEIK (SEQ |
| | MVTVSS (SEQ ID NO: 85) | ID NO: 49) |
| ARB34 | EVQLVESGGGLVQPGRSLRLSCAASGSTLSSYGMHWV | DIQMTQSPSSLSASVGDRVTITCRASQGIGSYLAWYQ |
| | RQAPGKGLEWVSAISYDASTIDYADSVEGRFTISRDNA | QKPGKAPKLLIYEASRLESGVPSRFSGSGSGTDFTLTIS |
| | KNSLYLQMNSLRAEDTAVYYCASEGVPYGMDVWGQG | SLQPEDVATYYCQQGYNAPITFGGGTKVEIK (SEQ ID |
| | TMVTVSS (SEQ ID NO: 86) | NO: 44) |
| ARB35 | QVQLQESGPGLVKPSQTLSLTCTVSGFSFSDFYWSWIRQ | EIVMTQSPATLSLSPGERATLSCRASQTIGTNLAWYQ |
| | PPGKGLEWIGYIDHTGSTDYNPSLKSRVTMSVDTSKNQ | QKPGQAPRLLIYDASTRANGIPARFSGSGSGTDFTLTI |
| | FSLKVNSVTAADTAVYYCAGDKYADGFDVWGQGTMV | SSLEPEDFAVYYCQQYAAPPLTFGGGTKVEIK (SEQ |
| | TVSS (SEQ ID NO: 87) | ID NO: 50) |
| ARB36 | QVQLQESGPGLVKPSQTLSLTCTVSGFSLSTSGVRWSWI | EIVMTQSPATLSLSPGERATLSCRASQSIRSDLAWYQ |
| | RQPPGKGLEWIGYINPSGTTDYNPSLKSRVTMSVDTSK | QKPGQAPRLLIYDASHRPAGIPARFSGSGSGTDFTLTI |
| | NQFSLKVNSVTAADTAVYYCARGGGYCSGGSCYDWY | SSLEPEDFAVYYCQQYGSVPRPTFGGGTKVEIK (SEQ |
| | FDLWGRGTLVTVSS (SEQ ID NO: 88) | ID NO: 51) |
| ARB37 | QVQLQESGPGLVKPSQTLSLTCTVSGFSLSTSGVRWSWI | EIVMTQSPATLSLSPGERATLSCRASQSIRSDLAWYQ |
| | RQPPGKGLEWIGYINPSGTTDYNPSLKSRVTMSVDTSK | QKPGQAPRLLIYDATSRAAGIPARFSGSGSGTDFTLTI |
| | NQFSLKVNSVTAADTAVYYCARGGGYCSGGSCYDWY | SSLEPEDFAVYYCQEYGSAPLTFGGGTKVEIK (SEQ |
| | FDLWGRGTLVTVSS (SEQ ID NO: 88) | ID NO: 52) |
| ARB38 | QVQLQESGPGLVKPSQTLSLTCTVSGFSFSGYSWSWIRQ | EIVMTQSPATLSLSPGERATLSCRASMGVSSNLAWYQ |
| | PPGKGLEWIGYITHSGTIDYNPSLKSRVTMSVDTSKNQF | QKPGQAPRLLIYDASNRAAGIPARFSGSGSGTDFTLTI |
| | SLKVNSVTAADTAVYYCAKPLDFGDYKDAFDIWGQGT | SSLEPEDFAVYYCQQYAEGPLTFGGGTKVEIK (SEQ |
| | MVTVSS (SEQ ID NO: 89) | ID NO: 53) |
| ARB39 | EVQLVESGGGLVQPGRSLRLSCAASGSTLSSYGMHWV | DIQMTQSPSSLSASVGDRVTITCRASQGIGSYLAWYQ |
| | RQAPGKGLEWVSAISYDASTIDYADSVEGRFTISRDNA | QKPGKAPKLLIYEASRLESGVPSRFSGSGSGTDFTLTIS |
| | KNSLYLQMNSLRAEDTAVYYCARDGISGIDYWGQGTL | SLQPEDVATYYCQQGYNAPITFGGGTKVEIK (SEQ ID |
| | VTVSS (SEQ ID NO: 90) | NO: 44) |

In some embodiments, the anti-FcγRIIβ antibodies comprise a VH or VL that comprises a glutamine as the N-terminal residue. In some embodiments, antibodies are provided wherein the glutamine (Q) is mutated to a glutamic acid (E) residue.

In some embodiment, the antibody is in a scFv format where the VH and VL are linked with a linker, such as those provided for herein and as illustrated in non-limiting embodiments in the table above.

In some embodiments, an anti-FcγRIIb antibody, or an antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53.

In some embodiments, an anti-FcγRIIb antibody, or an antigen-binding fragment thereof, comprises a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90.

In some embodiments, an anti-FcγRIIb comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53; and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90.

In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 18, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 54. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 19, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 55. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 20, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 56. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 21, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 57. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 22, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 58. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 23, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 59. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 24, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 60. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 25, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 61. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 26, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 62. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 27, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 63. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 28, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 64. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 29, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 65. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 30, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 66. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 31, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 67. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 32, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 68. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 33, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 69. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 34, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 70. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 35, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 71. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 36, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 72. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 37, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 73. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 38, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 74. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 39, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 75. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 40, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 76. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 41, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 77. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 42, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 78. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 43, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 79. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 44, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 80. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 45, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 81. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 46, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 80. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 47, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 82. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 48, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 83. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 44, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 84. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 49, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 85. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 44, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 86. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 50, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 87. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 51, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 88. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 52, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 88. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 53, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 89. In some embodiments, the anti-FcγRIIb antibody, or the antigen-binding fragment thereof, comprises a variable light chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 44, and a variable heavy chain comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 90.

In some embodiments, an anti-FcγRIIb antibody, or an antigen-binding fragment thereof, comprises a variable light chain comprising an amino acid sequence of any one of SEQ ID NOS: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 54.

In some embodiments, an anti-FcγRIIb antibody, or an antigen-binding fragment thereof, comprises a variable heavy chain comprising an amino acid sequence of any one of SEQ ID NOs: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90.

In some embodiments, an anti-FcγRIIb antibody, or an antigen-binding fragment thereof, comprises a variable light chain comprising an amino acid sequence selected from any one of SEQ ID NOs: 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53; and a variable heavy chain comprising an amino acid sequence selected from any one of SEQ ID NOs: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90.

In some embodiments, the anti-FcγRIIb antibody, or the antigen-binding fragment thereof, comprises a variable light chain comprising the amino acid sequence of SEQ ID NO: 18, and a variable heavy chain comprising the amino acid sequence of SEQ ID NO: 54. In some embodiments, the anti-FcγRIIb antibody, or the antigen-binding fragment thereof, comprises a variable light chain comprising the amino acid sequence of SEQ ID NO: 19, and a variable heavy chain comprising the amino acid sequence of SEQ ID NO: 55. In some embodiments, the anti-FcγRIIb antibody, or the antigen-binding fragment thereof, comprises a variable light chain comprising the amino acid sequence of SEQ ID NO: 20, and a variable heavy chain comprising the amino acid sequence of SEQ ID NO: 56. In some embodiments, the anti-FcγRIIb antibody, or the antigen-binding fragment thereof, comprises a variable light chain comprising the amino acid sequence of SEQ ID NO: 21, and a variable heavy chain comprising the amino acid sequence of SEQ ID NO: 57. In some embodiments, the anti-FcγRIIb antibody, or the antigen-binding fragment thereof, comprises a variable light chain comprising the amino acid sequence of SEQ ID NO: 22, and a variable heavy chain comprising the amino acid sequence of SEQ ID NO: 58. In some embodiments, the anti-FcγRIIb antibody, or the antigen-binding fragment thereof, comprises a variable light chain comprising the amino acid sequence of SEQ ID NO: 23, and a variable heavy chain comprising the amino acid sequence of SEQ ID NO: 59. In some embodiments, the anti-FcγRIIb antibody, or the antigen-binding fragment thereof, comprises a variable light chain comprising the amino acid sequence of SEQ ID NO: 24, and a variable heavy chain comprising the amino acid sequence of SEQ ID NO: 60. In some embodiments, the anti-FcγRIIb antibody, or the antigen-binding fragment thereof, comprises a variable light chain comprising the amino acid sequence of SEQ ID NO: 25, and a variable heavy chain comprising the amino acid sequence of SEQ ID NO: 61. In some embodiments, the anti-FcγRIIb antibody, or the antigen-binding fragment thereof, comprises a variable light chain comprising the amino acid sequence of SEQ ID NO: 26, and a variable heavy chain comprising the amino acid sequence of SEQ ID NO: 62. In some embodiments, the anti-FcγRIIb antibody, or the antigen-binding fragment thereof, comprises a variable light chain comprising the amino acid sequence of SEQ ID NO: 27, and a variable heavy chain comprising the amino acid sequence of SEQ ID NO: 63. In some embodiments, the anti-FcγRIIb antibody, or the antigen-binding fragment thereof, comprises a variable light chain comprising the amino acid sequence of SEQ ID NO: 28, and a variable heavy chain comprising the amino acid sequence of SEQ ID NO: 64. In some embodiments, the anti-FcγRIIb antibody, or the antigen-binding fragment thereof, comprises a variable light chain comprising the amino acid sequence of SEQ ID NO: 29, and a variable heavy chain comprising the amino acid sequence of SEQ ID NO: 65. In some embodiments, the anti-FcγRIIb antibody, or the antigen-binding fragment thereof, comprises a variable light chain comprising the amino acid sequence of SEQ ID NO: 30, and a variable heavy chain comprising the amino acid sequence of SEQ ID NO: 66. In some embodiments, the anti-FcγRIIb antibody, or the antigen-binding fragment thereof, comprises a variable light chain comprising the amino acid sequence of SEQ ID NO: 31, and a variable heavy chain comprising the amino acid sequence of SEQ ID NO: 67. In some embodiments, the anti-FcγRIIb antibody, or the antigen-binding fragment thereof, comprises a variable light chain comprising the amino acid sequence of SEQ ID NO: 32, and a variable heavy chain comprising the amino acid sequence of SEQ ID NO: 68. In some embodiments, the anti-FcγRIIb antibody, or the antigen-binding fragment thereof, comprises a variable light chain comprising the amino acid sequence of SEQ ID NO: 33, and a variable heavy chain comprising the amino acid sequence of SEQ ID NO: 69. In some embodiments, the anti-FcγRIIb antibody, or the antigen-binding fragment thereof, comprises a variable light chain comprising the amino acid sequence of SEQ ID NO: 34, and a variable heavy chain comprising the amino acid sequence of SEQ ID NO: 70. In some embodiments, the anti-FcγRIIb antibody, or the antigen-binding fragment thereof, comprises a variable light chain comprising the amino acid sequence of SEQ ID NO: 35, and a variable heavy chain comprising the amino acid sequence of SEQ ID NO: 71. In some embodiments, the anti-FcγRIIb antibody, or the antigen-binding fragment thereof, comprises a variable light chain comprising the amino acid sequence of SEQ ID NO: 36, and a variable heavy chain comprising the amino acid sequence of SEQ ID NO: 72. In some embodiments, the anti-FcγRIIb antibody, or the antigen-binding fragment thereof, comprises a variable light chain comprising the amino acid sequence of SEQ ID NO: 37, and a variable heavy chain comprising the amino acid sequence of SEQ ID NO: 73. In some embodiments, the anti-FcγRIIb antibody, or the antigen-binding fragment thereof, comprises a variable light chain comprising the amino acid sequence of SEQ ID NO: 38, and a variable heavy chain comprising the amino acid sequence of SEQ ID NO: 74. In some embodiments, the anti-FcγRIIb antibody, or the antigen-binding fragment thereof, comprises a variable light chain comprising the amino acid sequence of SEQ ID NO: 39, and a variable heavy chain comprising the amino acid sequence of SEQ ID NO: 75. In some embodiments, the anti-FcγRIIb antibody, or the antigen-binding fragment thereof, comprises a variable light chain comprising the amino acid sequence of SEQ ID NO: 40, and a variable heavy chain comprising the amino acid sequence of SEQ ID NO: 76. In some embodiments, the anti-FcγRIIb antibody, or the antigen-binding fragment thereof, comprises a variable light chain comprising the amino acid sequence of SEQ ID NO: 41, and a variable heavy chain comprising the amino acid sequence of SEQ ID NO: 77. In some embodiments, the anti-FcγRIIb antibody, or the antigen-binding fragment thereof, comprises a variable light chain comprising the amino acid sequence of SEQ ID NO: 42, and a variable heavy chain comprising the amino acid sequence of SEQ ID NO: 78. In some embodiments, the anti-FcγRIIb antibody, or the antigen-binding fragment thereof, comprises a variable light chain comprising the amino acid sequence of SEQ ID NO: 43, and a variable heavy chain comprising the amino acid sequence of SEQ ID NO: 79. In some embodiments, the anti-FcγRIIb antibody, or the antigen-binding fragment thereof, comprises a variable light chain comprising the amino acid sequence of SEQ ID NO: 44, and a variable heavy chain comprising the amino acid sequence of SEQ ID NO: 80. In some embodiments, the anti-FcγRIIb antibody, or the antigen-binding fragment thereof, comprises a variable light chain comprising the amino acid sequence of SEQ ID NO: 45, and a variable heavy chain comprising the amino acid sequence of SEQ ID NO: 81. In some embodiments, the anti-FcγRIIb antibody, or the antigen-binding fragment thereof, comprises a variable light chain comprising the amino acid sequence of SEQ ID NO: 46, and a variable heavy chain comprising the amino acid sequence of SEQ ID NO: 80. In some embodiments, the anti-FcγRIIb antibody, or the antigen-binding fragment thereof, comprises a variable light chain comprising the amino acid sequence of SEQ ID NO: 47, and a variable heavy chain comprising the amino acid sequence of SEQ ID NO: 82. In some embodiments, the anti-FcγRIIb antibody, or the antigen-binding fragment thereof, comprises a variable light chain comprising the amino acid sequence of SEQ ID NO: 48, and a variable heavy chain comprising the amino acid sequence of SEQ ID NO: 83. In some embodiments, the anti-FcγRIIb antibody, or the antigen-binding fragment thereof, comprises a variable light chain comprising the amino acid sequence of SEQ ID NO: 44, and a variable heavy chain comprising the amino acid sequence of SEQ ID NO: 84. In some embodiments, the anti-FcγRIIb antibody, or the antigen-binding fragment thereof, comprises a variable light chain comprising the amino acid sequence of SEQ ID NO: 49, and a variable heavy chain comprising the amino acid sequence of SEQ ID NO: 85. In some embodiments, the anti-FcγRIIb antibody, or the antigen-binding fragment thereof, comprises a variable light chain comprising the amino acid sequence of SEQ ID NO: 44, and a variable heavy chain comprising the amino acid sequence of SEQ ID NO: 86. In some embodiments, the anti-FcγRIIb antibody, or the antigen-binding fragment thereof, comprises a variable light chain comprising the amino acid sequence of SEQ ID NO: 50, and a variable heavy chain comprising the amino acid sequence of SEQ ID NO: 87. In some embodiments, the anti-FcγRIIb antibody, or the antigen-binding fragment thereof, comprises a variable light chain comprising the amino acid sequence of SEQ ID NO: 51, and a variable heavy chain comprising the amino acid sequence of SEQ ID NO: 88. In some embodiments, the anti-FcγRIIb antibody, or the antigen-binding fragment thereof, comprises a variable light chain comprising the amino acid sequence of SEQ ID NO: 52, and a variable heavy chain comprising the amino acid sequence of SEQ ID NO: 88. In some embodiments, the anti-FcγRIIb antibody, or the antigen-binding fragment thereof, comprises a variable light chain comprising the amino acid sequence of SEQ ID NO: 53, and a variable heavy chain comprising the amino acid sequence of SEQ ID NO: 89. In some embodiments, the anti-FcγRIIb antibody, or the antigen-binding fragment thereof, comprises a variable light chain comprising the amino acid sequence of SEQ ID NO: 44, and a variable heavy chain comprising the amino acid sequence of SEQ ID NO: 90.

In some embodiments, the anti-FcγRIIβ antibody comprises a VH and VL comprising the CDRs of the amino acid sequence as set forth in ARB1 to ARB39. Determining CDRs is routine and can be done according to the Kabat, Chothia, IMGT numbering systems, and the like. In some embodiments, the anti-FcγRIIβ antibody comprises a VH and VL comprising an amino acid sequence that is at least 90%, 91%, 92%, 93%, 94%, 95%, 96%, 97%, 98%, or 99% identical to the VH and VL pairs as set forth in ARB1 to ARB39. In some embodiments, the anti-PD-1 antibody comprises a VH and VL comprising an amino acid sequence that is at least 90%, 91%, 92%, 93%, 94%, 95%, 96%, 97%, 98%, or 99% identical to the VH and VL pairs as set forth in ARB1 to ARB39, provided that the CDRs are identical to ARB1 to ARB39. These can be collectively referred to as a variant of the VH and VL sequences provided for herein.

TABLE 13

| VH | HCDR1 | HCDR2 | HCDR3 | VL | LCDR1 | LCDR2 | LCDR3 |
|---|---|---|---|---|---|---|---|
| EVQLVESGGGLVQPG | DYHMS | SIDTE | DVGDW | DIVMTQSPLSLPVTPG | RSSRS | MASNR | MQALR |
| GSLRLSCAASRFTFS | (SEQ | GKTYY | YFDL | EPASISCRSSRSLVHG | LVHGS | AP | APFS |
| DYHMSWVRQAPGKGL | ID | PDSVK | (SEQ | SGDNYLHWYLQKPGQS | GDNYL | (SEQ | (SEQ |
| ELVASIDTEGKTYYP | NO: | G | ID | PQLLIYMASNRAPGVP | H | ID | ID |
| DSVKGRFTISRDNAK | 575) | (SEQ | NO: | DRFSGSGSGTDFTLKI | (SEQ | NO: | NO: |
| NSLYLQMNSLRAEDT | | ID | 577) | SRVEAEDVGVYYCMQA | ID | 582) | 583) |
| AVYYCARDVGDWYFD | | NO: | | LRAPFSFGGGTKVEIK | NO: | | |
| LWGRGTLVTVSS | | 576) | | (SEQ ID NO: 42) | 581) | | |
| (SEQ ID NO: 78) | | | | | | | |
| EVQLVESGGGLVQPG | SYGMH | AISYD | DGISG | DIQMTQSPSSLSASVG | RASQG | EASRL | QQGYN |
| RSLRLSCAASGSTLS | (SEQ | ASTID | IDY | DRVTITCRASQGIGSY | IGSYL | ES | APIT |
| SYGMHWVRQAPGKGL | ID | YADSV | (SEQ | LAWYQQKPGKAPKLLI | A | (SEQ | (SEQ |
| EWVSAISYDASTIDY | NO: | EG | ID | YEASRLESGVPSRFSG | (SEQ | ID | ID |
| ADSVEGRFTISRDNA | 578) | (SEQ | NO: | SGSGTDFTLTISSLQP | ID | NO: | NO: |
| KNSLYLQMNSLRAED | | ID | 580) | EDVATYYCQQGYNAPI | NO: | 585) | 586) |
| TAVYYCARDGISGID | | NO: | | TFGGGTKVEIK (SEQ | 584) | | |
| YWGQGTLVTVSS | | 579) | | ID NO: 44) | | | |
| (SEQ ID NO: 90) | | | | | | | |
| EVQLVESGGGLVQPG | RFTFS | IDTEG | ARDVG | DIVMTQSPLSLPVTPG | RSLVH | MAS | MQALR |
| GSLRLSCAASRFTFS | DYH | KT | DWYFD | EPASISCRSSRSLVHG | GSGDN | (SEQ | APFS |
| DYHMSWVRQAPGKGL | (SEQ | (SEQ | L | SGDNYLHWYLQKPGQS | Y | ID | (SEQ |
| ELVASIDTEGKTYYP | ID | ID | (SEQ | PQLLIYMASNRAPGVP | (SEQ | NO: | ID |
| DSVKGRFTISRDNAK | NO: | NO: | ID | DRFSGSGSGTDFTLKI | ID | 594) | NO: |
| NSLYLQMNSLRAEDT | 590) | 591) | NO: | SRVEAEDVGVYYCMQA | NO: | | 583) |
| AVYYCARDVGDWYFD | | | 592) | LRAPFSFGGGTKVEIK | 593) | | |
| LWGRGTLVTVSS | | | | (SEQ ID NO: 42) | | | |
| (SEQ ID NO: 78) | | | | | | | |
| QVQLQESGPGLVKPS | GVSLS | VHTSG | ARDAG | EIVMTQSPATLSLSPG | QSLSS | DAS | QQYAV |
| QTLSLTCTVSGVSLS | NARMG | ST | NWFDP | ERATLSCRASQSLSSS | SH | (SEQ | PPIT |
| NARMGWSWIRQPPGK | (SEQ | (SEQ | (SEQ | HLAWYQQKPGQAPRLL | (SEQ | ID | (SEQ |
| GLEWIGYVHTSGSTD | ID | ID | ID | IYDASIRVPGIPARFS | ID | NO: | ID |
| YNPSLKSRVTMSVDT | NO: | NO: | NO: | GSGSGTDFTLTISSLE | NO: | 599) | NO: |
| SKNQFSLKVNSVTAA | 595) | 596) | 597) | PEDFAVYYCQQYAVPP | 598) | | 600) |
| DTAVYYCARDAGNWE | | | | ITFGGGTKVEIK | | | |
| DPWGQGTLVTVSS | | | | (SEQ ID NO: 43) | | | |
| (SEQ ID NO: 79) | | | | | | | |
| EVQLVESGGGLVQPG | GFNFG | INQGG | ARDPG | DIQMTQSPSSLSASVG | QDISN | DAS | HQIYD |
| RSLRLSCAASGENFG | AFA | SEV | LPVSA | DRVTITCRASQDISNW | W | (SEQ | TPPT |
| AFAMHWVRQAPGKGL | (SEQ | (SEQ | FDI | LAWYQQKPGKAPKLLI | (SEQ | ID | (SEQ |
| EWVSAINQGGSEVDY | ID | ID | (SEQ | YDASSLVSGVPSRFSG | ID | NO: | ID |
| ADSVEGRFTISRDNA | NO: | NO: | ID | SGSGTDFTLTISSLQP | NO: | 599) | NO: |
| KNSLYLQMNSLRAED | 601) | 602) | NO | EDVATYYCHQIYDTPP | 604) | | 605) |
| TAVYYCARDPGLPVS | | | 603) | TFGGGTKVEIK | | | |
| AFDIWGLGTMVTVSS | | | | (SEQ ID NO: 47) | | | |
| (SEQ ID NO: 82) | | | | | | | |
| EVQLVESGGGLVQPG | GSTLS | ISYDA | ASEYG | DIQMTQSPSSLSASVG | QGIGS | EAS | QQGYN |
| RSLRLSCAASGSTLS | SYG | STI | LAFDQ | DRVTITCRASQGIGSY | Y | (SEQ | APIT |
| SYGMHWVRQAPGKGL | (SEQ | (SEQ | (SEQ | LAWYQQKPGKAPKLLI | (SEQ | ID | (SEQ |
| EWVSAISYDASTIDY | ID | ID | ID | YEASRLESGVPSRFSG | ID | NO: | ID |
| ADSVEGRFTISRDNA | NO: | NO: | NO: | SGSGTDFTLTISSLQP | NO: | 610) | NO: |
| KNSLYLQMNSLRAED | 606) | 607) | 608) | EDVATYYCQQGYNAPI | 609) | | 586) |
| TAVYYCASEYGLAFD | | | | TFGGGTKVEIK | | | |
| QWGQGTLVTVSS | | | | (SEQ ID NO: 44) | | | |
| (SEQ ID NO: 84) | | | | | | | |

In some embodiments, the anti-FcγRIIβ antibody comprises a CDR1 from any one of CDR1 of Table 13, a CDR2 from any one of CDR2 of Table 13, and a CDR3 from any one of CDR3 of Table 13. In some embodiments, the anti-FcγRIIβ antibody comprises a variable heavy (VH) chain comprising a heavy chain CDR1 (HCDR1) from any one of HCDR1 of Table 13, a heavy chain CDR2 (HCDR2) from any one of HCDR2 of Table 13, and a heavy chain CDR3 (HCDR3) from any one of HCDR3 of Table 13. In some embodiments, the anti-FcγRIIβ antibody comprises a variable light (VL) chain comprising a light chain CDR1 (LCDR1) from any one of LCDR1 of Table 13, a light chain CDR2 (LCDR2) from any one of LCDR2 of Table 13, and a light chain CDR3 (LCDR3) from any one of LCDR3 of Table 13. In some embodiments, the amino acid residues of the CDRs shown above contain mutations. In some embodiments, the CDRs contain 1, 2, 3, 4, 5, 6, 7, 8, 9, or 10 substitutions or mutations. In some embodiments, the substitution is a conservative substitution.

In some embodiments, the anti-FcγRIIβ antibody comprises the VH comprising the HCDR1 having an amino acid sequence of any one SEQ ID NOs: 575, 578, 590, 595, 601, or 606, the HCDR2 having an amino acid sequence of any one of SEQ ID NOs: 576, 579, 591, 596, 602, or 607, and the HCDR3 having an amino acid sequence of any one of SEQ ID NOs: 577, 580, 592, 597, 603, or 608; and the VL comprising the LCDR1 having an amino acid sequence of any one SEQ ID NOs: 581, 584, 593, 598, 604, or 609, the LCDR2 having an amino acid sequence of any one of SEQ ID NOs: 582, 585, 594, 599, or 610, and the LCDR3 having an amino acid sequence of any one of SEQ ID NOs: 583, 586, 600, or 605.

In some embodiments, the anti-FcγRIIβ antibody comprises the VH comprising the HCDR1 having an amino acid sequence of SEQ ID NO: 575, the HCDR2 having an amino acid sequence of SEQ ID NO: 576, and the HCDR3 having an amino acid sequence of SEQ ID NO: 577; and the VL comprising the LCDR1 having an amino acid sequence of SEQ ID NO: 581, the LCDR2 having an amino acid sequence of any one of SEQ ID NO: 582, and the LCDR3 having an amino acid sequence of SEQ ID NO: 583. In some embodiments, the anti-FcγRIIβ antibody comprises the VH comprising the HCDR1 having an amino acid sequence of SEQ ID NO: 578, the HCDR2 having an amino acid sequence of SEQ ID NO: 579, and the HCDR3 having an amino acid sequence of SEQ ID NO: 580; and the VL comprising the LCDR1 having an amino acid sequence of SEQ ID NO: 584, the LCDR2 having an amino acid sequence of any one of SEQ ID NO: 585, and the LCDR3 having an amino acid sequence of SEQ ID NO: 586. In some embodiments, the anti-FcγRIIβ antibody comprises the VH comprising the HCDR1 having an amino acid sequence of SEQ ID NO: 590, the HCDR2 having an amino acid sequence of SEQ ID NO: 591, and the HCDR3 having an amino acid sequence of SEQ ID NO: 592; and the VL comprising the LCDR1 having an amino acid sequence of SEQ ID NO: 593, the LCDR2 having an amino acid sequence of any one of SEQ ID NO: 594, and the LCDR3 having an amino acid sequence of SEQ ID NO: 583. In some embodiments, the anti-FcγRIIβ antibody comprises the VH comprising the HCDR1 having an amino acid sequence of SEQ ID NO: 595, the HCDR2 having an amino acid sequence of SEQ ID NO: 596, and the HCDR3 having an amino acid sequence of SEQ ID NO: 597; and the VL comprising the LCDR1 having an amino acid sequence of SEQ ID NO: 598, the LCDR2 having an amino acid sequence of any one of SEQ ID NO: 599, and the LCDR3 having an amino acid sequence of SEQ ID NO: 600. In some embodiments, the anti-FcγRIIβ antibody comprises the VH comprising the HCDR1 having an amino acid sequence of SEQ ID NO: 601, the HCDR2 having an amino acid sequence of SEQ ID NO: 602, and the HCDR3 having an amino acid sequence of SEQ ID NO: 603; and the VL comprising the LCDR1 having an amino acid sequence of SEQ ID NO: 604, the LCDR2 having an amino acid sequence of any one of SEQ ID NO: 599, and the LCDR3 having an amino acid sequence of SEQ ID NO: 605. In some embodiments, the anti-FcγRIIβ antibody comprises the VH comprising the HCDR1 having an amino acid sequence of SEQ ID NO: 606, the HCDR2 having an amino acid sequence of SEQ ID NO: 607, and the HCDR3 having an amino acid sequence of SEQ ID NO: 608; and the VL comprising the LCDR1 having an amino acid sequence of SEQ ID NO: 609, the LCDR2 having an amino acid sequence of any one of SEQ ID NO: 610, and the LCDR3 having an amino acid sequence of SEQ ID NO: 586.

In some embodiments, an anti-FcγRIIb antibody, or an antigen-binding fragment thereof, comprises the VH having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 78, wherein the amino acid sequence comprises the HCDR1 having an amino acid sequence of SEQ ID NO: 575; the HCDR2 having an amino acid sequence of SEQ ID NO: 576; and the HCDR3 having an amino acid sequence of SEQ ID NO: 577. In some embodiments, an anti-FcγRIIb antibody, or an antigen-binding fragment thereof, comprises the VH having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 90, wherein the amino acid sequence comprises the HCDR1 having an amino acid sequence of SEQ ID NO: 578; the HCDR2 having an amino acid sequence of SEQ ID NO: 579; and the HCDR3 having an amino acid sequence of SEQ ID NO: 580. In some embodiments, an anti-FcγRIIb antibody, or an antigen-binding fragment thereof, comprises the VH having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 78, wherein the amino acid sequence comprises the HCDR1 having an amino acid sequence of SEQ ID NO: 590; the HCDR2 having an amino acid sequence of SEQ ID NO: 591; and the HCDR3 having an amino acid sequence of SEQ ID NO: 592. In some embodiments, an anti-FcγRIIb antibody, or an antigen-binding fragment thereof, comprises the VH having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 79, wherein the amino acid sequence comprises the HCDR1 having an amino acid sequence of SEQ ID NO: 595; the HCDR2 having an amino acid sequence of SEQ ID NO: 596; and the HCDR3 having an amino acid sequence of SEQ ID NO: 597. In some embodiments, an anti-FcγRIIb antibody, or an antigen-binding fragment thereof, comprises the VH having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 82, wherein the amino acid sequence comprises the HCDR1 having an amino acid sequence of SEQ ID NO: 601; the HCDR2 having an amino acid sequence of SEQ ID NO: 602; and the HCDR3 having an amino acid sequence of SEQ ID NO: 603. In some embodiments, an anti-FcγRIIb antibody, or an antigen-binding fragment thereof, comprises the VH having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 84, wherein the amino acid sequence comprises the HCDR1 having an amino acid sequence of SEQ ID NO: 606; the HCDR2 having an amino acid sequence of SEQ ID NO: 607; and the HCDR3 having an amino acid sequence of SEQ ID NO: 608.

In some embodiments, an anti-FcγRIIb antibody, or an antigen-binding fragment thereof, comprises the VL having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 42, wherein the amino acid sequence comprises the LCDR1 having an amino acid sequence of SEQ ID NO: 581; the LCDR2 having an amino acid sequence of SEQ ID NO: 582; and the LCDR3 having an amino acid sequence of SEQ ID NO: 583. In some embodiments, an anti-FcγRIIb antibody, or an antigen-binding fragment thereof, comprises the VL having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 44, wherein the amino acid sequence comprises the LCDR1 having an amino acid sequence of SEQ ID NO: 584; the LCDR2 having an amino acid sequence of SEQ ID NO: 585; and the LCDR3 having an amino acid sequence of SEQ ID NO: 586. In some embodiments, an anti-FcγRIIb antibody, or an antigen-binding fragment thereof, comprises the VL having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 42, wherein the amino acid sequence comprises the LCDR1 having an amino acid sequence of SEQ ID NO: 593; the LCDR2 having an amino acid sequence of SEQ ID NO: 594; and the LCDR3 having an amino acid sequence of SEQ ID NO: 583. In some embodiments, an anti-FcγRIIb antibody, or an antigen-binding fragment thereof, comprises the VL having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 43, wherein the amino acid sequence comprises the LCDR1 having an amino acid sequence of SEQ ID NO: 598; the LCDR2 having an amino acid sequence of SEQ ID NO: 599; and the LCDR3 having an amino acid sequence of SEQ ID NO: 600. In some embodiments, an anti-FcγRIIb antibody, or an antigen-binding fragment thereof, comprises the VL having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 47, wherein the amino acid sequence comprises the LCDR1 having an amino acid sequence of SEQ ID NO: 604; the LCDR2 having an amino acid sequence of SEQ ID NO: 599; and the LCDR3 having an amino acid sequence of SEQ ID NO: 605. In some embodiments, an anti-FcγRIIb antibody, or an antigen-binding fragment thereof, comprises the VL having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 44, wherein the amino acid sequence comprises the LCDR1 having an amino acid sequence of SEQ ID NO: 609; the LCDR2 having an amino acid sequence of SEQ ID NO: 610; and the LCDR3 having an amino acid sequence of SEQ ID NO: 586.

In some embodiments, the anti-FcγRIIβ antibody, or the antigen-binding fragment thereof, is in a Fab or IgG format.

In some embodiments, the anti-FcγRIIβ antibody, or the antigen-binding fragment thereof, is in an scFv format. In some embodiments, the anti-FcγRIIβ antibody, or the antigen-binding fragment thereof, comprises a polypeptide comprising any one of the amino acid sequences provided in Table 7 below.

TABLE 7

| Molecule | |
|---|---|
| ID | scFv Sequence |
| ARB1 | DIVMTQSPLSLPVTPGEPASISCRSSQSLVHGSGYTFLHWYLQKPGQSPQLLIYDAVTRATGVPDRFSGSGSGTDFTLK |
| | ISRVEAEDVGVYYCMQTTEFPYTFGGGTKVEIKGGGGSGGGGSGGGGSGGGGSEVQLVESGGGLVQPGGSLRLSCA |
| | ASAFTFSAHSMSWVRQAPGKGLELVASISPSGSVTYYPDSVKGRFTISRDNAKNSLYLQMNSLRAEDTAVYYCARDS |
| | GSYRDAFDIWGQGTMVTVSS (SEQ ID NO: 91) |
| ARB2 | DIVMTQSPLSLPVTPGEPASISCRSSQSLLHSTGYNFLHWYLQKPGQSPQLLIYDASKRAPGVPDRFSGSGSGTDFTLKI |
| | SRVEAEDVGVYYCMQTVELPFTFGGGTKVEIKGGGGSGGGGSGGGGSGGGGSEVQLVESGGGLVQPGGSLRLSCA |
| | ASGFTFAGHAMSWVRQAPGKGLELVASISPSGSTTYYPDSVKGRFTISRDNAKNSLYLQMNSLRAEDTAVYYCARD |
| | GDSSDAFDIWGQGTMVTVSS (SEQ ID NO: 92) |
| ARB3 | EIVMTQSPATLSLSPGERATLSCRASQSVDRHLAWYQQKPGQAPRLLIYDVSNRAPGIPARFSGSGSGTDFTLTISSLE |
| | PEDFAVYYCQQYGWPSITFGGGTKVEIKGGGGSGGGGSGGGGSGGGGSQVQLQESGPGLVKPSQTLSLTCTVSGASI |
| | STIDYSWSWIRQPPGKGLEWIGYIDESGRIDYNPSLKSRVTMSVDTSKNQFSLKVNSVTAADTAVYYCAREGQWGQF |
| | DYWGQGTLVTVSS (SEQ ID NO: 93) |
| ARB4 | DIVMTQSPLSLPVTPGEPASISCRSSQSLLSSYGYHNLHWYLQKPGQSPQLLIYDAYVRATGVPDRFSGSGSGTDFTLK |
| | ISRVEAEDVGVYYCMQSKELPYTFGGGTKVEIKGGGGSGGGGSGGGGSGGGGSEVQLVESGGGLVQPGGSLRLSCA |
| | ASGFTFSNHAMSWVRQAPGKGLELVASINPSGSVTYYPDSVKGRFTISRDNAKNSLYLQMNSLRAEDTAVYYCARD |
| | GDYGDYLDYWGQGTLVTVSS (SEQ ID NO: 94) |
| ARB5 | DIQMTQSPSSVSASVGDRVTITCRASQDIGSNLAWYQQKPGKAPKLLIYSGSTLQSGVPSRFSGSGSGTDFTLTISSLQP |
| | EDFANYYCQQTSSSPPYTFGGGTKVEIKGGGGSGGGGSGGGGSGGGGSQVQLVQSGAEVKKPGASVKVSCKVSGD |
| | TFTSNAIHWVRQAPGKGLEWMGGIVAGSGHTIYAQKFQGRVTMTEDTSTDTAYMELSSLKSEDTAVYYCAREGAE |
| | YNGFDPWGQGTLVTVSS (SEQ ID NO: 95) |
| ARB6 | DIQMTQSPSSVSASVGDRVTITCRASQNIGNWLAWYQQKPGKAPKLLIYAASNLQRGVPSRFSGSGSGTDFTLTISSL |
| | QPEDFANYYCQQYHNIPITFGGGTKVEIKGGGGSGGGGSGGGGSGGGGSQVQLVQSGAEVKKPGASVKVSCKVSGF |
| | TFTSSVIHWVRQAPGKGLEWMGGISPRGESTIYAQKFQGRVTMTEDTSTDTAYMELSSLKSEDTAVYYCAREGAST |
| | GAFDIWGQGTMVTVSS (SEQ ID NO: 96) |
| ARB7 | DIQMTQSPSSVSASVGDRVTITCRASQGIGTYLAWYQQKPGKAPKLLIYDASILGSGVPSRFSGSGSGTDFTLTISSLQP |
| | EDFANYYCQHYGTSPPTFGGGTKVEIKGGGGSGGGGSGGGGSGGGGSQVQLVQSGAEVKKPGASVKVSCKVSGYT |
| | LTGNAIHWVRQAPGKGLEWMGGIIPSIGTAIYAQKFQGRVTMTEDTSTDTAYMELSSLKSEDTAVYYCAKDRSDIGD |
| | IFDYWGQGTLVTVSS (SEQ ID NO: 97) |
| ARB8 | DIVMTQSPLSLPVTPGEPASISCRSSQSLLHSTGYNFLHWYLQKPGQSPQLLIYDTFHRATGVPDRFSGSGSGTDFTLKI |
| | SRVEAEDVGVYYCMQSLQPRFTFGGGTKVEIKGGGGSGGGGSGGGGSGGGGSEVQLVESGGGLVQPGGSLRLSCA |
| | ASGFTFTTHSMSWVRQAPGKGLELVASINPAGSITYYPDSVKGRFTISRDNAKNSLYLQMNSLRAEDTAVYYCARDN |
| | NYGYGDHFDYWGQGTLVTVSS (SEQ ID NO: 98) |
| ARB9 | DIVMTQSPLSLPVTPGEPASISCRSSQSLLHSTGYNYLHWYLQKPGQSPQLLIYDTSTRASGVPDRFSGSGSGTDFTLKI |
| | SRVEAEDVGVYYCMQTFHLPFTFGGGTKVEIKGGGGSGGGGSGGGGSGGGGSEVQLVESGGGLVQPGGSLRLSCA |
| | ASGFTFGDHVMSWVRQAPGKGLELVASISPSGDVTYYPDSVKGRFTISRDNAKNSLYLQMNSLRAEDTAVYYCTTD |
| | GDSDAFDIWGQGTMVTVSS (SEQ ID NO: 99) |
| ARB10 | DIVMTQSPLSLPVTPGEPASISCRSSQSLLHSSGYNYLHWYLQKPGQSPQLLIYDTTNRATGVPDRFSGSGSGTDFTLK |
| | ISRVEAEDVGVYYCMQTTQLPYTFGGGTKVEIKGGGGSGGGGSGGGGSGGGGSEVQLVESGGGLVQPGGSLRLSCA |
| | ASGLTFSNHAMSWVRQAPGKGLELVASINPSGSVTYYPDSVKGRFTISRDNAKNSLYLQMNSLRAEDTAVYYCTAD |
| | DYGDYLDYWGQGTLVTVSS (SEQ ID NO: 100) |
| ARB11 | DIQMTQSPSSVSASVGDRVTITCRASQGIGRSLAWYQQKPGKAPKLLIYKDTERASGVPSRFSGSGSGTDFTLTISSLQ |
| | PEDFANYYCQQVHTFPPTFGGGTKVEIKGGGGSGGGGSGGGGSGGGGSQVQLVQSGAEVKKPGASVKVSCKVSGY |
| | TFSNYVIHWVRQAPGKGLEWMGGIVAGSGHTIYAQKFQGRVTMTEDTSTDTAYMELSSLKSEDTAVYYCATESAA |
| | GNWFDPWGQGTLVTVSS (SEQ ID NO: 101) |
| ARB12 | DIVMTQSPLSLPVTPGEPASISCRSSQSLLHVTGYNYLHWYLQKPGQSPQLLIYETSKRAPGVPDRFSGSGSGTDFTLK |
| | ISRVEAEDVGVYYCMQTTHWPSTFGGGTKVEIKGGGGSGGGGSGGGGSGGGGSEVQLVESGGGLVQPGGSLRLSC |
| | AASGFTFSDHTMSWVRQAPGKGLELVASINPSGSVTYYPDSVKGRFTISRDNAKNSLYLQMNSLRAEDTAVYYCAR |
| | DGGYGDYFDYWGQGTLVTVSS (SEQ ID NO: 102) |
| ARB13 | DIQMTQSPSSVSASVGDRVTITCRASQSIGTNLAWYQQKPGKAPKLLIYDASKRPTGVPSRFSGSGSGTDFTLTISSLQ |
| | PEDFANYYCQQGYDIPLTFGGGTKVEIKGGGGSGGGGSGGGGSGGGGSQVQLVQSGAEVKKPGASVKVSCKVSGTP |
| | LPATIHWVRQAPGKGLEWMGGINPSGATIYAQKFQGRVTMTEDTSTDTAYMELSSLKSEDTAVYYCAKDSDVAAA |
| | GSFFDYWGQGTLVTVSS (SEQ ID NO: 103) |
| ARB14 | DIQMTQSPSSVSASVGDRVTITCRASQNIGDRLAWYQQKPGKAPKLLIYQDRNRPSGVPSRFSGSGSGTDFTLTISSLQ |
| | PEDFANYYCQQYATSPFTFGGGTKVEIKGGGGSGGGGSGGGGSGGGGSQVQLVQSGAEVKKPGASVKVSCKVSGY |
| | TFRNYAIHWVRQAPGKGLEWMGGISPSGSTTIYAQKFQGRVTMTEDTSTDTAYMELSSLKSEDTAVYYCARESAEN |
| | YGDYLDYWGQGTLVTVSS (SEQ ID NO: 104) |
| ARB15 | DIQMTQSPSSVSASVGDRVTITCRASQNIDTYLAWYQQKPGKAPKLLIYEGTKRPSGVPSRFSGSGSGTDFTLTISSLQ |
| | PEDFANYYCQQWDNLPLTFGGGTKVEIKGGGGSGGGGSGGGGSGGGGSQVQLVQSGAEVKKPGASVKVSCKVSGI |
| | DLTTSAIHWVRQAPGKGLEWMGGIAIGSGHTIYAQKFQGRVTMTEDTSTDTAYMELSSLKSEDTAVYYCARDGNFG |
| | DYIEYWGQGTLVTVSS (SEQ ID NO: 105) |
| ARB16 | DIQMTQSPSSVSASVGDRVTITCRASESISSHLAWYQQKPGKAPKLLIYAGSSRATGVPSRFSGSGSGTDFTLTISSLQP |
| | EDFANYYCHQYDTLPFTFGGGTKVEIKGGGGSGGGGSGGGGSGGGGSQVQLVQSGAEVKKPGASVKVSCKVSGHT |
| | FTGYFIHWVRQAPGKGLEWMGGMDPISGATIYAQKFQGRVTMTEDTSTDTAYMELSSLKSEDTAVYYCAREGTTID |
| | AFDIWGQGTMVTVSS (SEQ ID NO: 106) |
| ARB17 | DIVMTQSPLSLPVTPGEPASISCRSSRSLVHGSGDNYLHWYLQKPGQSPQLLIYMTSNRAPGVPDRFSGSGSGTDFTLK |
| | ISRVEAEDVGVYYCMQSGHWPPTFGGGTKVEIKGGGGSGGGGSGGGGSGGGGSEVQLVESGGGLVQPGGSLRLSC |
| | AASGLMFSTSTMSWVRQAPGKGLELVASINPSGSVTYYPDSVKGRFTISRDNAKNSLYLQMNSLRAEDTAVYYCAR |
| | EDGDSRGEAFDIWGQGTMVTVSS (SEQ ID NO: 107) |
| ARB18 | DIQMTQSPSSVSASVGDRVTITCRASQNIDTWLAWYQQKPGKAPKLLIYKASTLASGVPSRFSGSGSGTDFTLTISSLQ |
| | PEDFANYYCQQYNEVPLTFGGGTKVEIKGGGGSGGGGSGGGGSGGGGSQVQLVQSGAEVKKPGASVKVSCKVSGI |
| | DLTTSAIHWVRQAPGKGLEWMGGINPGTGSTIYAQKFQGRVTMTEDTSTDTAYMELSSLKSEDTAVYYCAKEVAAT |
| | GAYYYWGQGTLVTVSS (SEQ ID NO: 108) |
| ARB19 | DIVMTQSPLSLPVTPGEPASISCRSSQSLLHSTGYNYLHWYLQKPGQSPQLLIYDATNRASGVPDRFSGSGSGTDFTLK |
| | ISRVEAEDVGVYYCQQYAASPPTFGGGTKVEIKGGGGSGGGGSGGGGSGGGGSEVQLVESGGGLVQPGGSLRLSCA |
| | ASGFPFSDYVMSWVRQAPGKGLELVASISPSGSTTYYPDSVKGRFTISRDNAKNSLYLQMNSLRAEDTAVYYCVRSG |
| | EWTDAFDIWGQGTLVTVSS (SEQ ID NO: 109) |
| ARB20 | DIQMTQSPSSVSASVGDRVTITCRASQDISTYLAWYQQKPGKAPKLLIYDTSNRATGIPSRFSGSGSGTDFTLTISSLQP |
| | EDFANYYCQQSAKIPFTFGGGTKVEIKGGGGSGGGGSGGGGSGGGGSQVQLVQSGAEVKKPGASVKVSCKVSGYTF |
| | SDYVIHWVRQAPGKGLEWMGGINPYSGHTIYAQKFQGRVTMTEDTSTDTAYMELSSLKSEDTAVYYCAKELDTAG |
| | NAFDIWGQGTMVTVSS (SEQ ID NO: 110) |
| ARB21 | EIVMTQSPATLSLSPGERATLSCRASHSVTSNLAWYQQKPGQAPRLLIYGASSRVPGIPARFSGSGSGTDFTLTISSLEP |
| | EDFAVYYCQQYDNRPITFGGGTKVEIKGGGGSGGGGSGGGGSGGGGSQVQLQESGPGLVKPSQTLSLTCTVSGASV |
| | RDHYWSWIRQPPGKGLEWIGYAHYSGITDYNPSLKSRVTMSVDTSKNQFSLKVNSVTAADTAVYYCAIYSSGWWE |
| | DYWGQGTLVTVSS (SEQ ID NO: 111) |
| ARB22 | DIQMTQSPSSVSASVGDRVTITCRASQSIAPLAWYQQKPGKAPKLLIYAASTLQPGVPSRFSGSGSGTDFTLTISSLQPE |
| | DFANYYCLQYHVLPITFGGGTKVEIKGGGGSGGGGSGGGGSGGGGSQVQLVQSGAEVKKPGASVKVSCKVSGYPFP |
| | SYDIHWVRQAPGKGLEWMGGMNPTTGDTIYAQKFQGRVTMTEDTSTDTAYMELSSLKSEDTAVYYCARETGGGE |
| | MAFDIWGQGTMVTVSS (SEQ ID NO: 112) |
| ARB23 | EIVMTQSPATLSLSPGERATLSCRASQNIGTNLAWYQQKPGQAPRLLIYDASKRPTGIPARFSGSGSGTDFTLTISSLEP |
| | EDFAVYYCQQYGTTPFTFGGGTKVEIKGGGGSGGGGSGGGGSGGGGSQVQLQESGPGLVKPSQTLSLTCTVSEYAIR |
| | SDYTWSWIRQPPGKGLEWIGYIHHSGLTDYNPSLKSRVTMSVDTSKNQFSLKVNSVTAADTAVYYCARVRYSSSSE |
| | GWFDPWGQGTLVTVSS (SEQ ID NO: 113) |
| ARB24 | EIVMTQSPATLSLSPGERATLSCRASESIGSNLAWYQQKPGQAPRLLIYDASNRATGIPARFSGSGSGTDFTLTISSLEP |
| | EDFAVYYCQQYDHWPLTFGGGTKVEIKGGGGSGGGGSGGGGSGGGGSQVQLQESGPGLVKPSQTLSLTCTVSGASI |
| | STSDTWSWIRQPPGKGLEWIGYIHHSGITDYNPSLKSRVTMSVDTSKNQFSLKVNSVTAADTAVYYCARGGSSGNW |
| | YLLDYWGQGTLVTVSS (SEQ ID NO: 114) |
| ARB25 | DIVMTQSPLSLPVTPGEPASISCRSSRSLVHGSGDNYLHWYLQKPGQSPQLLIYMASNRAPGVPDRFSGSGSGTDFTL |
| | KISRVEAEDVGVYYCMQALRAPFSFGGGTKVEIKGGGGSGGGGSGGGGSGGGGSEVQLVESGGGLVQPGGSLRLSC |
| | AASRFTFSDYHMSWVRQAPGKGLELVASIDTEGKTYYPDSVKGRFTISRDNAKNSLYLQMNSLRAEDTAVYYCARD |
| | VGDWYFDLWGRGTLVTVSS (SEQ ID NO: 115) |
| ARB26 | EIVMTQSPATLSLSPGERATLSCRASQSLSSSHLAWYQQKPGQAPRLLIYDASIRVPGIPARFSGSGSGTDFTLTISSLEP |
| | EDFAVYYCQQYAVPPITFGGGTKVEIKGGGGSGGGGSGGGGSGGGGSQVQLQESGPGLVKPSQTLSLTCTVSGVSLS |
| | NARMGWSWIRQPPGKGLEWIGYVHTSGSTDYNPSLKSRVTMSVDTSKNQFSLKVNSVTAADTAVYYCARDAGNW |
| | FDPWGQGTLVTVSS (SEQ ID NO: 116) |
| ARB27 | DIQMTQSPSSLSASVGDRVTITCRASQGIGSYLAWYQQKPGKAPKLLIYEASRLESGVPSRFSGSGSGTDFTLTISSLQP |
| | EDVATYYCQQGYNAPITFGGGTKVEIKGGGGSGGGGSGGGGSGGGGSEVQLVESGGGLVQPGRSLRLSCAASGSTL |
| | SSYGMHWVRQAPGKGLEWVSAISYDASTIDYADSVEGRFTISRDNAKNSLYLQMNSLRAEDTAVYYCARDLLGYG |
| | MDVWGQGTMVTVSS (SEQ ID NO: 117) |
| ARB28 | DIQMTQSPSSVSASVGDRVTITCRASQDIGNWLAWYQQKPGKAPKLLIYDASTLDTGVPSRFSGSGSGTDFTLTISSLQ |
| | PEDFANYYCLQDYSYPLTFGGGTKVEIKGGGGSGGGGSGGGGSGGGGSQVQLVQSGAEVKKPGASVKVSCKVSGS |
| | AFTSNDIHWVRQAPGKGLEWMGGINPRSGATIYAQKFQGRVTMTEDTSTDTAYMELSSLKSEDTAVYYCARALSDD |
| | SSGYDAFDIWGQGTMVTVSS (SEQ ID NO: 118) |
| ARB29 | DIQMTQSPSSLSASVGDRVTITCRASQDISNWLAWYQQKPGKAPKLLIYEASRLESGVPSRFSGSGSGTDFTLTISSLQ |
| | PEDVATYYCQQGYNAPITFGGGTKVEIKGGGGSGGGGSGGGGSGGGGSEVQLVESGGGLVQPGRSLRLSCAASGST |
| | LSSYGMHWVRQAPGKGLEWVSAISYDASTIDYADSVEGRFTISRDNAKNSLYLQMNSLRAEDTAVYYCARDLLGY |
| | GMDVWGQGTMVTVSS (SEQ ID NO: 119) |
| ARB30 | DIQMTQSPSSLSASVGDRVTITCRASQDISNWLAWYQQKPGKAPKLLIYDASSLVSGVPSRFSGSGSGTDFTLTISSLQ |
| | PEDVATYYCHQIYDTPPTFGGGTKVEIKGGGGSGGGGSGGGGSGGGGSEVQLVESGGGLVQPGRSLRLSCAASGFNF |
| | GAFAMHWVRQAPGKGLEWVSAINQGGSEVDYADSVEGRFTISRDNAKNSLYLQMNSLRAEDTAVYYCARDPGLPV |
| | SAFDIWGLGTMVTVSS (SEQ ID NO: 120) |
| ARB31 | DIQMTQSPSSLSASVGDRVTITCRASQAISGSLAWYQQKPGKAPKLLIYATSRLESGVPSRFSGSGSGTDFTLTISSLQP |
| | EDVATYYCQQSGSTPITFGGGTKVEIKGGGGSGGGGSGGGGSGGGGSEVQLVESGGGLVQPGRSLRLSCAASGFTFE |
| | NYGMHWVRQAPGKGLEWVSAISYDASTIDYADSVEGRFTISRDNAKNSLYLQMNSLRAEDTAVYYCASEYGLAFD |
| | QWGQGTLVTVSS (SEQ ID NO: 121) |
| ARB32 | DIQMTQSPSSLSASVGDRVTITCRASQGIGSYLAWYQQKPGKAPKLLIYEASRLESGVPSRFSGSGSGTDFTLTISSLQP |
| | EDVATYYCQQGYNAPITFGGGTKVEIKGGGGSGGGGSGGGGSGGGGSEVQLVESGGGLVQPGRSLRLSCAASGSTL |
| | SSYGMHWVRQAPGKGLEWVSAISYDASTIDYADSVEGRFTISRDNAKNSLYLQMNSLRAEDTAVYYCASEYGLAFD |
| | QWGQGTLVTVSS (SEQ ID NO: 122) |
| ARB33 | DIQMTQSPSSLSASVGDRVTITCRASQDIAGWLAWYQQKPGKAPKLLIYDASTLQGGVPSRFSGSGSGTDFTLTISSLQ |
| | PEDVATYYCQQSYTAPLNFGGGTKVEIKGGGGSGGGGSGGGGSGGGGSEVQLVESGGGLVQPGRSLRLSCAASGLT |
| | FSNHGMHWVRQAPGKGLEWVSAISSSADIVDYADSVEGRFTISRDNAKNSLYLQMNSLRAEDTAVYYCASEGVPYG |
| | MDVWGQGTMVTVSS (SEQ ID NO: 123) |
| ARB34 | DIQMTQSPSSLSASVGDRVTITCRASQGIGSYLAWYQQKPGKAPKLLIYEASRLESGVPSRFSGSGSGTDFTLTISSLQP |
| | EDVATYYCQQGYNAPITFGGGTKVEIKGGGGSGGGGSGGGGSGGGGSEVQLVESGGGLVQPGRSLRLSCAASGSTL |
| | SSYGMHWVRQAPGKGLEWVSAISYDASTIDYADSVEGRFTISRDNAKNSLYLQMNSLRAEDTAVYYCASEGVPYG |
| | MDVWGQGTMVTVSS (SEQ ID NO: 124) |
| ARB35 | EIVMTQSPATLSLSPGERATLSCRASQTIGTNLAWYQQKPGQAPRLLIYDASTRANGIPARFSGSGSGTDFTLTISSLEP |
| | EDFAVYYCQQYAAPPLTFGGGTKVEIKGGGGGGGGSGGGGSGGGGSQVQLQESGPGLVKPSQTLSLTCTVSGESFS |
| | DFYWSWIRQPPGKGLEWIGYIDHTGSTDYNPSLKSRVTMSVDTSKNQFSLKVNSVTAADTAVYYCAGDKYADGFD |
| | VWGQGTMVTVSS (SEQ ID NO: 125) |
| ARB36 | EIVMTQSPATLSLSPGERATLSCRASQSIRSDLAWYQQKPGQAPRLLIYDASHRPAGIPARFSGSGSGTDFTLTISSLEP |
| | EDFAVYYCQQYGSVPRPTFGGGTKVEIKGGGGSGGGGSGGGGSGGGGSQVQLQESGPGLVKPSQTLSLTCTVSGFSL |
| | STSGVRWSWIRQPPGKGLEWIGYINPSGTTDYNPSLKSRVTMSVDTSKNQFSLKVNSVTAADTAVYYCARGGGYCS |
| | GGSCYDWYFDLWGRGTLVTVSS (SEQ ID NO: 126) |
| ARB37 | EIVMTQSPATLSLSPGERATLSCRASQSIRSDLAWYQQKPGQAPRLLIYDATSRAAGIPARFSGSGSGTDFTLTISSLEP |
| | EDFAVYYCQEYGSAPLTFGGGTKVEIKGGGGSGGGGSGGGGSGGGGSQVQLQESGPGLVKPSQTLSLTCTVSGFSLS |
| | TSGVRWSWIRQPPGKGLEWIGYINPSGTTDYNPSLKSRVTMSVDTSKNQFSLKVNSVTAADTAVYYCARGGGYCSG |
| | GSCYDWYFDLWGRGTLVTVSS (SEQ ID NO: 127) |
| ARB38 | EIVMTQSPATLSLSPGERATLSCRASMGVSSNLAWYQQKPGQAPRLLIYDASNRAAGIPARFSGSGSGTDFTLTISSLE |
| | PEDFAVYYCQQYAEGPLTFGGGTKVEIKGGGGSGGGGSGGGGSGGGGSQVQLQESGPGLVKPSQTLSLTCTVSGESF |
| | SGYSWSWIRQPPGKGLEWIGYITHSGTIDYNPSLKSRVTMSVDTSKNQFSLKVNSVTAADTAVYYCAKPLDFGDYK |
| | DAFDIWGQGTMVTVSS (SEQ ID NO: 128) |
| ARB39 | DIQMTQSPSSLSASVGDRVTITCRASQGIGSYLAWYQQKPGKAPKLLIYEASRLESGVPSRFSGSGSGTDFTLTISSLQP |
| | EDVATYYCQQGYNAPITFGGGTKVEIKGGGGSGGGGSGGGGSGGGGSEVQLVESGGGLVQPGRSLRLSCAASGSTL |
| | SSYGMHWVRQAPGKGLEWVSAISYDASTIDYADSVEGRFTISRDNAKNSLYLQMNSLRAEDTAVYYCARDGISGID |
| | YWGQGTLVTVSS (SEQ ID NO: 129) |

In some embodiments, an anti-FcγRIIb antibody, or an antigen-binding fragment thereof, comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, or 129, provided that the HCDRs and the LCDRs are identical to the CDRs of the antibody as set forth herein or as determined by KABAT, Chothia, or IMGT.

In some embodiments, an anti-FcγRIIb antibody, or an antigen-binding fragment thereof, comprises the amino acid sequence of any one of SEQ ID NO: 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, or 129.

In some embodiments, the anti-FcγRIIβ antibody is conjugated to an Fc polypeptide, such as those provided herein. In some embodiments, the anti-FcγRIIβ antibody is conjugated to an Fc polypeptide as provided for herein. In some embodiments, the Fc comprises “AAA” mutations, LALA mutations, P238D mutation, or G237E and P238E. In some embodiments, the Fc comprises an amino acid sequence of SEQ ID NO: 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 543, 544, 545, 546, 683, 684, 685, 686, 687, 688, 689, or 690. In some embodiments, the Fc comprises the amino acid sequence of SEQ ID NO: 543.

In some embodiments, the anti-FcγRIIβ antibody conjugated to the Fc polypeptide comprises the heavy chain of any one amino acid sequence provided in Table 34 below, which can be expressed with the light chain as provided for in Table 34.

TABLE 34

| Molecule | | |
|---|---|---|
| ID | Heavy Chain | Light Chain |
| ARB40 | EVQLVESGGGLVQPGGSLRLSCAASRFTFSDYHMSWVRQAPGKGLELVA | DIVMTQSPLSLPVTPGEPASISCR |
| | SIDTEGKTYYPDSVKGRFTISRDNAKNSLYLQMNSLRAEDTAVYYCARD | SSRSLVHGSGDNYLHWYLQKPGQS |
| | VGDWYFDLWGRGTLVTVSSASTKGPSVFPLAPSSKSTSGGTAALGCLVK | PQLLIYMASNRAPGVPDRFSGSGS |
| | DYFPEPVTVSWNSGALTSGVHTFPAVLQSSGLYSLSSVVTVPSSSLGTQ | GTDFTLKISRVEAEDVGVYYCMQA |
| | TYICNVNHKPSNTKVDKKVEPKSCDKTHTCPPCPAPEAAGAPSVFLFPP | LRAPFSFGGGTKVEIKRTVAAPSV |
| | KPKDTLMISRTPEVTCVVVDVSHEDPEVKFNWYVDGVEVHNAKTKPREE | FIFPPSDEQLKSGTASVVCLLNNF |
| | QYNSTYRVVSVLTVLHQDWLNGKEYKCKVSNKALPAPIEKTISKAKGQP | YPREAKVQWKVDNALQSGNSQESV |
| | REPQVYTLPPSRDELTKNQVSLTCLVKGFYPSDIAVEWESNGQPENNYK | TEQDSKDSTYSLSSTLTLSKADYE |
| | TTPPVLDSDGSFFLYSKLTVDKSRWQQGNVFSCSVMHEALHNHYTQKSL | KHKVYACEVTHQGLSSPVTKSFNR |
| | SLSPG (SEQ ID NO: 611) | GEC (SEQ ID NO: 647) |
| ARB41 | EVQLVESGGGLVQPGGSLRLSCAASRFTFSDYHMSWVRQAPGKGLELVA | DIVMTQSPLSLPVTPGEPASISCR |
| | SIDTEGKTYYPDSVKGRFTISRDNAKNSLYLQMNSLRAEDTAVYYCARD | SSRSLVHGSGDNYLHWYLQKPGQS |
| | VGDWYFDLWGRGTLVTVSSASTKGPSVFPLAPSSKSTSGGTAALGCLVK | PQLLIYMASNRAPGVPDRFSGSGS |
| | DYFPEPVTVSWNSGALTSGVHTFPAVLQSSGLYSLSSVVTVPSSSLGTQ | GTDFTLKISRVEAEDVGVYYCMQA |
| | TYICNVNHKPSNTKVDKKVEPKSCDKTHTCPPCPAPEAAGAPSVFLFPP | LRAPFSFGGGTKVEIKRTVAAPSV |
| | KPKDTLMISRTPEVTCVVVDVSHEDPEVKFNWYVDGVEVHNAKTKPREE | FIFPPSDEQLKSGTASVVCLLNNF |
| | QYNSTYRVVSVLTVLHQDWLNGKEYKCKVSNKALPAPIEKTISKAKGQP | YPREAKVQWKVDNALQSGNSQESV |
| | REPQVYTLPPSRDELTKNQVSLTCLVKGFYPSDIAVEWESNGQPENNYK | TEQDSKDSTYSLSSTLTLSKADYE |
| | TTPPVLDSDGSFFLYSKLTVDKSRWQQGNVFSCSVLHEALHSHYTQKSL | KHKVYACEVTHQGLSSPVTKSFNR |
| | SLSPG (SEQ ID NO: 612) | GEC (SEQ ID NO: 648) |
| ARB42 | EVQLVESGGGLVQPGGSLRLSCAASRFTFSDYHMSWVRQAPGKGLELVA | DIVMTQSPLSLPVTPGEPASISCR |
| | SIDTEGKTYYPDSVKGRFTISRDNAKNSLYLQMNSLRAEDTAVYYCARD | SSRSLVHGSGDNYLHWYLQKPGQS |
| | VGDWYFDLWGRGTLVTVSSASTKGPSVFPLAPSSKSTSGGTAALGCLVK | PQLLIYMASNRAPGVPDRFSGSGS |
| | DYFPEPVTVSWNSGALTSGVHTFPAVLOSSGLYSLSSVVTVPSSSLGTQ | GTDFTLKISRVEAEDVGVYYCMQA |
| | TYICNVNHKPSNTKVDKKVEPKSCDKTHTCPPCPAPEAAGAPSVFLFPP | LRAPFSFGGGTKVEIKRTVAAPSV |
| | KPKDTLYITREPEVTCVVVDVSHEDPEVKFNWYVDGVEVHNAKTKPREE | FIFPPSDEQLKSGTASVVCLLNNF |
| | QYNSTYRVVSVLTVLHQDWLNGKEYKCKVSNKALPAPIEKTISKAKGQP | YPREAKVQWKVDNALQSGNSQESV |
| | REPQVYTLPPSRDELTKNQVSLTCLVKGFYPSDIAVEWESNGQPENNYK | TEQDSKDSTYSLSSTLTLSKADYE |
| | TTPPVLDSDGSFFLYSKLTVDKSRWQQGNVFSCSVMHEALHNHYTQKSL | KHKVYACEVTHQGLSSPVTKSFNR |
| | SLSPG (SEQ ID NO: 613) | GEC (SEQ ID NO: 649) |
| ARB43 | EVQLVESGGGLVQPGGSLRLSCAASRFTFSDYHMSWVRQAPGKGLELVA | DIVMTQSPLSLPVTPGEPASISCR |
| | SIDTEGKTYYPDSVKGRFTISRDNAKNSLYLQMNSLRAEDTAVYYCARD | SSRSLVHGSGDNYLHWYLQKPGQS |
| | VGDWYFDLWGRGTLVTVSSASTKGPSVFPLAPSSKSTSGGTAALGCLVK | PQLLIYMASNRAPGVPDRFSGSGS |
| | DYFPEPVTVSWNSGALTSGVHTFPAVLQSSGLYSLSSVVTVPSSSLGTQ | GTDFTLKISRVEAEDVGVYYCMQA |
| | TYICNVNHKPSNTKVDKKVEPKSCDKTHTCPPCPAPELLGGDSVFLFPP | LRAPFSFGGGTKVEIKRTVAAPSV |
| | KPKDTLMISRTPEVTCVVVDVSHEDPEVKFNWYVDGVEVHNAKTKPREE | FIFPPSDEQLKSGTASVVCLLNNF |
| | QYNSTYRVVSVLTVLHQDWLNGKEYKCKVSNKALPAPIEKTISKAKGQP | YPREAKVQWKVDNALQSGNSQESV |
| | REPQVYTLPPSRDELTKNQVSLTCLVKGFYPSDIAVEWESNGQPENNYK | TEQDSKDSTYSLSSTLTLSKADYE |
| | TTPPVLDSDGSFFLYSKLTVDKSRWQQGNVFSCSVMHEALHNHYTQKSL | KHKVYACEVTHQGLSSPVTKSFNR |
| | SLSPG (SEQ ID NO: 614) | GEC (SEQ ID NO: 650) |
| ARB44 | EVQLVESGGGLVQPGGSLRLSCAASRFTFSDYHMSWVRQAPGKGLELVA | DIVMTQSPLSLPVTPGEPASISCR |
| | SIDTEGKTYYPDSVKGRFTISRDNAKNSLYLQMNSLRAEDTAVYYCARD | SSRSLVHGSGDNYLHWYLQKPGQS |
| | VGDWYFDLWGRGTLVTVSSASTKGPSVFPLAPSSKSTSGGTAALGCLVK | PQLLIYMASNRAPGVPDRFSGSGS |
| | DYFPEPVTVSWNSGALTSGVHTFPAVLQSSGLYSLSSVVTVPSSSLGTQ | GTDFTLKISRVEAEDVGVYYCMQA |
| | TYICNVNHKPSNTKVDKKVEPKSCDKTHTCPPCPAPELLGGDSVFLFPP | LRAPFSFGGGTKVEIKRTVAAPSV |
| | KPKDTLMISRTPEVTCVVVDVSHEDPEVKFNWYVDGVEVHNAKTKPREE | FIFPPSDEQLKSGTASVVCLLNNF |
| | QYNSTYRVVSVLTVLHQDWLNGKEYKCKVSNKALPAPIEKTISKAKGQP | YPREAKVQWKVDNALQSGNSQESV |
| | REPQVYTLPPSRDELTKNQVSLTCLVKGFYPSDIAVEWESNGQPENNYK | TEQDSKDSTYSLSSTLTLSKADYE |
| | TTPPVLDSDGSFFLYSKLTVDKSRWQQGNVFSCSVLHEALHSHYTQKSL | KHKVYACEVTHQGLSSPVTKSFNR |
| | SLSPG (SEQ ID NO: 615) | GEC (SEQ ID NO: 651) |
| ARB45 | EVQLVESGGGLVQPGGSLRLSCAASRFTFSDYHMSWVRQAPGKGLELVA | DIVMTQSPLSLPVTPGEPASISCR |
| | SIDTEGKTYYPDSVKGRFTISRDNAKNSLYLQMNSLRAEDTAVYYCARD | SSRSLVHGSGDNYLHWYLQKPGQS |
| | VGDWYFDLWGRGTLVTVSSASTKGPSVFPLAPSSKSTSGGTAALGCLVK | PQLLIYMASNRAPGVPDRFSGSGS |
| | DYFPEPVTVSWNSGALTSGVHTFPAVLQSSGLYSLSSVVTVPSSSLGTQ | GTDFTLKISRVEAEDVGVYYCMQA |
| | TYICNVNHKPSNTKVDKKVEPKSCDKTHTCPPCPAPELLGGDSVFLFPP | LRAPFSFGGGTKVEIKRTVAAPSV |
| | KPKDTLYITREPEVTCVVVDVSHEDPEVKFNWYVDGVEVHNAKTKPREE | FIFPPSDEQLKSGTASVVCLLNNF |
| | QYNSTYRVVSVLTVLHQDWLNGKEYKCKVSNKALPAPIEKTISKAKGQP | YPREAKVQWKVDNALQSGNSQESV |
| | REPQVYTLPPSRDELTKNQVSLTCLVKGFYPSDIAVEWESNGQPENNYK | TEQDSKDSTYSLSSTLTLSKADYE |
| | TTPPVLDSDGSFFLYSKLTVDKSRWQQGNVFSCSVMHEALHNHYTQKSL | KHKVYACEVTHQGLSSPVTKSFNR |
| | SLSPG (SEQ ID NO: 616) | GEC (SEQ ID NO: 652) |
| ARB46 | EVQLVESGGGLVQPGGSLRLSCAASRFTFSDYHMSWVRQAPGKGLELVA | DIVMTQSPLSLPVTPGEPASISCR |
| | SIDTEGKTYYPDSVKGRFTISRDNAKNSLYLQMNSLRAEDTAVYYCARD | SSRSLVHGSGDNYLHWYLQKPGQS |
| | VGDWYFDLWGRGTLVTVSSASTKGPSVFPLAPSSKSTSGGTAALGCLVK | PQLLIYMASNRAPGVPDRFSGSGS |
| | DYFPEPVTVSWNSGALTSGVHTFPAVLQSSGLYSLSSVVTVPSSSLGTQ | GTDFTLKISRVEAEDVGVYYCMQA |
| | TYICNVNHKPSNTKVDKKVEPKSCDKTHTCPPCPAPELLGEESVFLFPP | LRAPFSFGGGTKVEIKRTVAAPSV |
| | KPKDTLMISRTPEVTCVVVDVSHEDPEVKFNWYVDGVEVHNAKTKPREE | FIFPPSDEQLKSGTASVVCLLNNF |
| | QYNSTYRVVSVLTVLHQDWLNGKEYKCKVSNKALPAPIEKTISKAKGQP | YPREAKVQWKVDNALQSGNSQESV |
| | REPQVYTLPPSRDELTKNQVSLTCLVKGFYPSDIAVEWESNGQPENNYK | TEQDSKDSTYSLSSTLTLSKADYE |
| | TTPPVLDSDGSFFLYSKLTVDKSRWQQGNVFSCSVMHEALHNHYTQKSL | KHKVYACEVTHQGLSSPVTKSFNR |
| | SLSPG (SEQ ID NO: 617) | GEC (SEQ ID NO: 653) |
| ARB47 | EVQLVESGGGLVQPGGSLRLSCAASRFTFSDYHMSWVRQAPGKGLELVA | DIVMTQSPLSLPVTPGEPASISCR |
| | SIDTEGKTYYPDSVKGRFTISRDNAKNSLYLQMNSLRAEDTAVYYCARD | SSRSLVHGSGDNYLHWYLQKPGQS |
| | VGDWYFDLWGRGTLVTVSSASTKGPSVFPLAPSSKSTSGGTAALGCLVK | PQLLIYMASNRAPGVPDRFSGSGS |
| | DYFPEPVTVSWNSGALTSGVHTFPAVLQSSGLYSLSSVVTVPSSSLGTQ | GTDFTLKISRVEAEDVGVYYCMQA |
| | TYICNVNHKPSNTKVDKKVEPKSCDKTHTCPPCPAPELLGEESVFLFPP | LRAPFSFGGGTKVEIKRTVAAPSV |
| | KPKDTLMISRTPEVTCVVVDVSHEDPEVKFNWYVDGVEVHNAKTKPREE | FIFPPSDEQLKSGTASVVCLLNNF |
| | QYNSTYRVVSVLTVLHQDWLNGKEYKCKVSNKALPAPIEKTISKAKGQP | YPREAKVQWKVDNALQSGNSQESV |
| | REPQVYTLPPSRDELTKNQVSLTCLVKGFYPSDIAVEWESNGQPENNYK | TEQDSKDSTYSLSSTLTLSKADYE |
| | TTPPVLDSDGSFFLYSKLTVDKSRWQQGNVFSCSVLHEALHSHYTQKSL | KHKVYACEVTHQGLSSPVTKSFNR |
| | SLSPG (SEQ ID NO: 618) | GEC (SEQ ID NO: 654) |
| ARB48 | EVQLVESGGGLVQPGGSLRLSCAASRFTFSDYHMSWVRQAPGKGLELVA | DIVMTQSPLSLPVTPGEPASISCR |
| | SIDTEGKTYYPDSVKGRFTISRDNAKNSLYLQMNSLRAEDTAVYYCARD | SSRSLVHGSGDNYLHWYLQKPGQS |
| | VGDWYFDLWGRGTLVTVSSASTKGPSVFPLAPSSKSTSGGTAALGCLVK | PQLLIYMASNRAPGVPDRFSGSGS |
| | DYFPEPVTVSWNSGALTSGVHTFPAVLQSSGLYSLSSVVTVPSSSLGTQ | GTDFTLKISRVEAEDVGVYYCMQA |
| | TYICNVNHKPSNTKVDKKVEPKSCDKTHTCPPCPAPELLGEESVFLFPP | LRAPFSFGGGTKVEIKRTVAAPSV |
| | KPKDTLYITREPEVTCVVVDVSHEDPEVKFNWYVDGVEVHNAKTKPREE | FIFPPSDEQLKSGTASVVCLLNNF |
| | QYNSTYRVVSVLTVLHQDWLNGKEYKCKVSNKALPAPIEKTISKAKGQP | YPREAKVQWKVDNALQSGNSQESV |
| | REPQVYTLPPSRDELTKNQVSLTCLVKGFYPSDIAVEWESNGQPENNYK | TEQDSKDSTYSLSSTLTLSKADYE |
| | TTPPVLDSDGSFFLYSKLTVDKSRWQQGNVFSCSVMHEALHNHYTQKSL | KHKVYACEVTHQGLSSPVTKSFNR |
| | SLSPG (SEQ ID NO: 619) | GEC (SEQ ID NO: 655) |
| ARB49 | QVQLQESGPGLVKPSQTLSLTCTVSGVSLSNARMGWSWIRQPPGKGLEW | EIVMTQSPATLSLSPGERATLSCR |
| | IGYVHTSGSTDYNPSLKSRVTMSVDTSKNQFSLKVNSVTAADTAVYYCA | ASQSLSSSHLAWYQQKPGQAPRLL |
| | RDAGNWFDPWGQGTLVTVSSASTKGPSVFPLAPSSKSTSGGTAALGCLV | IYDASIRVPGIPARFSGSGSGTDF |
| | KDYFPEPVTVSWNSGALTSGVHTFPAVLQSSGLYSLSSVVTVPSSSLGT | TLTISSLEPEDFAVYYCQQYAVPP |
| | QTYICNVNHKPSNTKVDKKVEPKSCDKTHTCPPCPAPEAAGAPSVFLFP | ITFGGGTKVEIKRTVAAPSVFIFP |
| | PKPKDTLMISRTPEVTCVVVDVSHEDPEVKFNWYVDGVEVHNAKTKPRE | PSDEQLKSGTASVVCLLNNFYPRE |
| | EQYNSTYRVVSVLTVLHQDWLNGKEYKCKVSNKALPAPIEKTISKAKGQ | AKVQWKVDNALQSGNSQESVTEQD |
| | PREPQVYTLPPSRDELTKNQVSLTCLVKGFYPSDIAVEWESNGQPENNY | SKDSTYSLSSTLTLSKADYEKHKV |
| | KTTPPVLDSDGSFFLYSKLTVDKSRWQQGNVFSCSVMHEALHNHYTQKS | YACEVTHQGLSSPVTKSFNRGEC |
| | LSLSPG (SEQ ID NO: 620) | (SEQ ID NO: 656) |
| ARB50 | QVQLQESGPGLVKPSQTLSLTCTVSGVSLSNARMGWSWIRQPPGKGLEW | EIVMTQSPATLSLSPGERATLSCR |
| | IGYVHTSGSTDYNPSLKSRVTMSVDTSKNQFSLKVNSVTAADTAVYYCA | ASQSLSSSHLAWYQQKPGQAPRLL |
| | RDAGNWFDPWGQGTLVTVSSASTKGPSVFPLAPSSKSTSGGTAALGCLV | IYDASIRVPGIPARFSGSGSGTDF |
| | KDYFPEPVTVSWNSGALTSGVHTFPAVLQSSGLYSLSSVVTVPSSSLGT | TLTISSLEPEDFAVYYCQQYAVPP |
| | QTYICNVNHKPSNTKVDKKVEPKSCDKTHTCPPCPAPEAAGAPSVFLFP | ITFGGGTKVEIKRTVAAPSVFIFP |
| | PKPKDTLMISRTPEVTCVVVDVSHEDPEVKFNWYVDGVEVHNAKTKPRE | PSDEQLKSGTASVVCLLNNFYPRE |
| | EQYNSTYRVVSVLTVLHQDWLNGKEYKCKVSNKALPAPIEKTISKAKGQ | AKVQWKVDNALQSGNSQESVTEQD |
| | PREPQVYTLPPSRDELTKNQVSLTCLVKGFYPSDIAVEWESNGQPENNY | SKDSTYSLSSTLTLSKADYEKHKV |
| | KTTPPVLDSDGSFFLYSKLTVDKSRWQQGNVFSCSVLHEALHSHYTQKS | YACEVTHQGLSSPVTKSFNRGEC |
| | LSLSPG (SEQ ID NO: 621) | (SEQ ID NO: 657) |
| ARB51 | QVQLQESGPGLVKPSQTLSLTCTVSGVSLSNARMGWSWIRQPPGKGLEW | EIVMTQSPATLSLSPGERATLSCR |
| | IGYVHTSGSTDYNPSLKSRVTMSVDTSKNQFSLKVNSVTAADTAVYYCA | ASQSLSSSHLAWYQQKPGQAPRLL |
| | RDAGNWFDPWGQGTLVTVSSASTKGPSVFPLAPSSKSTSGGTAALGCLV | IYDASIRVPGIPARFSGSGSGTDF |
| | KDYFPEPVTVSWNSGALTSGVHTFPAVLQSSGLYSLSSVVTVPSSSLGT | TLTISSLEPEDFAVYYCQQYAVPP |
| | QTYICNVNHKPSNTKVDKKVEPKSCDKTHTCPPCPAPEAAGAPSVFLFP | ITFGGGTKVEIKRTVAAPSVFIFP |
| | PKPKDTLYITREPEVTCVVVDVSHEDPEVKFNWYVDGVEVHNAKTKPRE | PSDEQLKSGTASVVCLLNNFYPRE |
| | EQYNSTYRVVSVLTVLHQDWLNGKEYKCKVSNKALPAPIEKTISKAKGQ | AKVQWKVDNALQSGNSQESVTEQD |
| | PREPQVYTLPPSRDELTKNQVSLTCLVKGFYPSDIAVEWESNGQPENNY | SKDSTYSLSSTLTLSKADYEKHKV |
| | KTTPPVLDSDGSFFLYSKLTVDKSRWQQGNVFSCSVMHEALHNHYTQKS | YACEVTHQGLSSPVTKSFNRGEC |
| | LSLSPG (SEQ ID NO: 622) | (SEQ ID NO: 658) |
| ARB52 | QVQLQESGPGLVKPSQTLSLTCTVSGVSLSNARMGWSWIRQPPGKGLEW | EIVMTQSPATLSLSPGERATLSCR |
| | IGYVHTSGSTDYNPSLKSRVTMSVDTSKNQFSLKVNSVTAADTAVYYCA | ASQSLSSSHLAWYQQKPGQAPRLL |
| | RDAGNWFDPWGQGTLVTVSSASTKGPSVFPLAPSSKSTSGGTAALGCLV | IYDASIRVPGIPARFSGSGSGTDF |
| | KDYFPEPVTVSWNSGALTSGVHTFPAVLQSSGLYSLSSVVTVPSSSLGT | TLTISSLEPEDFAVYYCQQYAVPP |
| | QTYICNVNHKPSNTKVDKKVEPKSCDKTHTCPPCPAPELLGGDSVFLFP | ITFGGGTKVEIKRTVAAPSVFIFP |
| | PKPKDTLMISRTPEVTCVVVDVSHEDPEVKFNWYVDGVEVHNAKTKPRE | PSDEQLKSGTASVVCLLNNFYPRE |
| | EQYNSTYRVVSVLTVLHQDWLNGKEYKCKVSNKALPAPIEKTISKAKGQ | AKVQWKVDNALQSGNSQESVTEQD |
| | PREPQVYTLPPSRDELTKNQVSLTCLVKGFYPSDIAVEWESNGQPENNY | SKDSTYSLSSTLTLSKADYEKHKV |
| | KTTPPVLDSDGSFFLYSKLTVDKSRWQQGNVFSCSVMHEALHNHYTQKS | YACEVTHQGLSSPVTKSFNRGEC |
| | LSLSPG (SEQ ID NO: 623) | (SEQ ID NO: 659) |
| ARB53 | QVQLQESGPGLVKPSQTLSLTCTVSGVSLSNARMGWSWIRQPPGKGLEW | EIVMTQSPATLSLSPGERATLSCR |
| | IGYVHTSGSTDYNPSLKSRVTMSVDTSKNQFSLKVNSVTAADTAVYYCA | ASQSLSSSHLAWYQQKPGQAPRLL |
| | RDAGNWFDPWGQGTLVTVSSASTKGPSVFPLAPSSKSTSGGTAALGCLV | IYDASIRVPGIPARFSGSGSGTDE |
| | KDYFPEPVTVSWNSGALTSGVHTFPAVLQSSGLYSLSSVVTVPSSSLGT | TLTISSLEPEDFAVYYCQQYAVPP |
| | QTYICNVNHKPSNTKVDKKVEPKSCDKTHTCPPCPAPELLGGDSVFLFP | ITFGGGTKVEIKRTVAAPSVFIFP |
| | PKPKDTLMISRTPEVTCVVVDVSHEDPEVKFNWYVDGVEVHNAKTKPRE | PSDEQLKSGTASVVCLLNNFYPRE |
| | EQYNSTYRVVSVLTVLHQDWLNGKEYKCKVSNKALPAPIEKTISKAKGQ | AKVQWKVDNALQSGNSQESVTEQD |
| | PREPQVYTLPPSRDELTKNQVSLTCLVKGFYPSDIAVEWESNGQPENNY | SKDSTYSLSSTLTLSKADYEKHKV |
| | KTTPPVLDSDGSFFLYSKLTVDKSRWQQGNVFSCSVLHEALHSHYTQKS | YACEVTHQGLSSPVTKSFNRGEC |
| | LSLSPG (SEQ ID NO: 624) | (SEQ ID NO: 660) |
| ARB54 | QVQLQESGPGLVKPSQTLSLTCTVSGVSLSNARMGWSWIRQPPGKGLEW | EIVMTQSPATLSLSPGERATLSCR |
| | IGYVHTSGSTDYNPSLKSRVTMSVDTSKNQFSLKVNSVTAADTAVYYCA | ASQSLSSSHLAWYQQKPGQAPRLL |
| | RDAGNWFDPWGQGTLVTVSSASTKGPSVFPLAPSSKSTSGGTAALGCLV | IYDASIRVPGIPARFSGSGSGTDE |
| | KDYFPEPVTVSWNSGALTSGVHTFPAVLQSSGLYSLSSVVTVPSSSLGT | TLTISSLEPEDFAVYYCQQYAVPP |
| | QTYICNVNHKPSNTKVDKKVEPKSCDKTHTCPPCPAPELLGGDSVFLFP | ITFGGGTKVEIKRTVAAPSVFIFP |
| | PKPKDTLYITREPEVTCVVVDVSHEDPEVKFNWYVDGVEVHNAKTKPRE | PSDEQLKSGTASVVCLLNNFYPRE |
| | EQYNSTYRVVSVLTVLHQDWLNGKEYKCKVSNKALPAPIEKTISKAKGQ | AKVQWKVDNALQSGNSQESVTEQD |
| | PREPQVYTLPPSRDELTKNQVSLTCLVKGFYPSDIAVEWESNGQPENNY | SKDSTYSLSSTLTLSKADYEKHKV |
| | KTTPPVLDSDGSFFLYSKLTVDKSRWQQGNVFSCSVMHEALHNHYTQKS | YACEVTHQGLSSPVTKSFNRGEC |
| | LSLSPG (SEQ ID NO: 625) | (SEQ ID NO: 661) |
| ARB55 | QVQLQESGPGLVKPSQTLSLTCTVSGVSLSNARMGWSWIRQPPGKGLEW | EIVMTQSPATLSLSPGERATLSCR |
| | IGYVHTSGSTDYNPSLKSRVTMSVDTSKNQFSLKVNSVTAADTAVYYCA | ASQSLSSSHLAWYQQKPGQAPRLL |
| | RDAGNWFDPWGQGTLVTVSSASTKGPSVFPLAPSSKSTSGGTAALGCLV | IYDASIRVPGIPARFSGSGSGTDF |
| | KDYFPEPVTVSWNSGALTSGVHTFPAVLQSSGLYSLSSVVTVPSSSLGT | TLTISSLEPEDFAVYYCQQYAVPP |
| | QTYICNVNHKPSNTKVDKKVEPKSCDKTHTCPPCPAPELLGEESVFLFP | ITFGGGTKVEIKRTVAAPSVFIFP |
| | PKPKDTLMISRTPEVTCVVVDVSHEDPEVKFNWYVDGVEVHNAKTKPRE | PSDEQLKSGTASVVCLLNNFYPRE |
| | EQYNSTYRVVSVLTVLHQDWLNGKEYKCKVSNKALPAPIEKTISKAKGQ | AKVQWKVDNALQSGNSQESVTEQD |
| | PREPQVYTLPPSRDELTKNQVSLTCLVKGFYPSDIAVEWESNGQPENNY | SKDSTYSLSSTLTLSKADYEKHKV |
| | KTTPPVLDSDGSFFLYSKLTVDKSRWQQGNVFSCSVMHEALHNHYTQKS | YACEVTHQGLSSPVTKSFNRGEC |
| | LSLSPG (SEQ ID NO: 626) | (SEQ ID NO: 662) |
| ARB56 | QVQLQESGPGLVKPSQTLSLTCTVSGVSLSNARMGWSWIRQPPGKGLEW | EIVMTQSPATLSLSPGERATLSCR |
| | IGYVHTSGSTDYNPSLKSRVTMSVDTSKNQFSLKVNSVTAADTAVYYCA | ASQSLSSSHLAWYQQKPGQAPRLL |
| | RDAGNWFDPWGQGTLVTVSSASTKGPSVFPLAPSSKSTSGGTAALGCLV | IYDASIRVPGIPARFSGSGSGTDF |
| | KDYFPEPVTVSWNSGALTSGVHTFPAVLQSSGLYSLSSVVTVPSSSLGT | TLTISSLEPEDFAVYYCQQYAVPP |
| | QTYICNVNHKPSNTKVDKKVEPKSCDKTHTCPPCPAPELLGEESVFLFP | ITFGGGTKVEIKRTVAAPSVFIFP |
| | PKPKDTLMISRTPEVTCVVVDVSHEDPEVKFNWYVDGVEVHNAKTKPRE | PSDEQLKSGTASVVCLLNNFYPRE |
| | EQYNSTYRVVSVLTVLHQDWLNGKEYKCKVSNKALPAPIEKTISKAKGQ | AKVQWKVDNALQSGNSQESVTEQD |
| | PREPQVYTLPPSRDELTKNQVSLTCLVKGFYPSDIAVEWESNGQPENNY | SKDSTYSLSSTLTLSKADYEKHKV |
| | KTTPPVLDSDGSFFLYSKLTVDKSRWQQGNVFSCSVLHEALHSHYTQKS | YACEVTHQGLSSPVTKSFNRGEC |
| | LSLSPG (SEQ ID NO: 627) | (SEQ ID NO: 663) |
| ARB57 | QVQLQESGPGLVKPSQTLSLTCTVSGVSLSNARMGWSWIRQPPGKGLEW | EIVMTQSPATLSLSPGERATLSCR |
| | IGYVHTSGSTDYNPSLKSRVTMSVDTSKNQFSLKVNSVTAADTAVYYCA | ASQSLSSSHLAWYQQKPGQAPRLL |
| | RDAGNWFDPWGQGTLVTVSSASTKGPSVFPLAPSSKSTSGGTAALGCLV | IYDASIRVPGIPARFSGSGSGTDF |
| | KDYFPEPVTVSWNSGALTSGVHTFPAVLQSSGLYSLSSVVTVPSSSLGT | TLTISSLEPEDFAVYYCQQYAVPP |
| | QTYICNVNHKPSNTKVDKKVEPKSCDKTHTCPPCPAPELLGEESVFLFP | ITFGGGTKVEIKRTVAAPSVFIFP |
| | PKPKDTLYITREPEVTCVVVDVSHEDPEVKFNWYVDGVEVHNAKTKPRE | PSDEQLKSGTASVVCLLNNFYPRE |
| | EQYNSTYRVVSVLTVLHQDWLNGKEYKCKVSNKALPAPIEKTISKAKGQ | AKVQWKVDNALQSGNSQESVTEQD |
| | PREPQVYTLPPSRDELTKNQVSLTCLVKGFYPSDIAVEWESNGQPENNY | SKDSTYSLSSTLTLSKADYEKHKV |
| | KTTPPVLDSDGSFFLYSKLTVDKSRWQQGNVFSCSVMHEALHNHYTQKS | YACEVTHQGLSSPVTKSFNRGEC |
| | LSLSPG (SEQ ID NO: 628) | (SEQ ID NO: 664) |
| ARB58 | EVQLVESGGGLVQPGRSLRLSCAASGFNFGAFAMHWVRQAPGKGLEWVS | DIQMTQSPSSLSASVGDRVTITCR |
| | AINQGGSEVDYADSVEGRFTISRDNAKNSLYLQMNSLRAEDTAVYYCAR | ASQDISNWLAWYQQKPGKAPKLLI |
| | DPGLPVSAFDIWGLGTMVTVSSASTKGPSVFPLAPSSKSTSGGTAALGC | YDASSLVSGVPSRFSGSGSGTDFT |
| | LVKDYFPEPVTVSWNSGALTSGVHTFPAVLQSSGLYSLSSVVTVPSSSL | LTISSLQPEDVATYYCHQIYDTPP |
| | GTQTYICNVNHKPSNTKVDKKVEPKSCDKTHTCPPCPAPEAAGAPSVFL | TFGGGTKVEIKRTVAAPSVFIFPP |
| | FPPKPKDTLMISRTPEVTCVVVDVSHEDPEVKFNWYVDGVEVHNAKTKP | SDEQLKSGTASVVCLLNNFYPREA |
| | REEQYNSTYRVVSVLTVLHQDWLNGKEYKCKVSNKALPAPIEKTISKAK | KVQWKVDNALQSGNSQESVTEQDS |
| | GQPREPQVYTLPPSRDELTKNQVSLTCLVKGFYPSDIAVEWESNGQPEN | KDSTYSLSSTLTLSKADYEKHKVY |
| | NYKTTPPVLDSDGSFFLYSKLTVDKSRWQQGNVFSCSVMHEALHNHYTQ | ACEVTHQGLSSPVTKSFNRGEC |
| | KSLSLSPG (SEQ ID NO: 629) | (SEQ ID NO: 665) |
| ARB59 | EVQLVESGGGLVQPGRSLRLSCAASGENFGAFAMHWVRQAPGKGLEWVS | DIQMTQSPSSLSASVGDRVTITCR |
| | AINQGGSEVDYADSVEGRFTISRDNAKNSLYLQMNSLRAEDTAVYYCAR | ASQDISNWLAWYQQKPGKAPKLLI |
| | DPGLPVSAFDIWGLGTMVTVSSASTKGPSVFPLAPSSKSTSGGTAALGC | YDASSLVSGVPSRFSGSGSGTDFT |
| | LVKDYFPEPVTVSWNSGALTSGVHTFPAVLQSSGLYSLSSVVTVPSSSL | LTISSLQPEDVATYYCHQIYDTPP |
| | GTQTYICNVNHKPSNTKVDKKVEPKSCDKTHTCPPCPAPEAAGAPSVFL | TFGGGTKVEIKRTVAAPSVFIFPP |
| | FPPKPKDTLMISRTPEVTCVVVDVSHEDPEVKFNWYVDGVEVHNAKTKP | SDEQLKSGTASVVCLLNNFYPREA |
| | REEQYNSTYRVVSVLTVLHQDWLNGKEYKCKVSNKALPAPIEKTISKAK | KVQWKVDNALQSGNSQESVTEQDS |
| | GQPREPQVYTLPPSRDELTKNQVSLTCLVKGFYPSDIAVEWESNGQPEN | KDSTYSLSSTLTLSKADYEKHKVY |
| | NYKTTPPVLDSDGSFFLYSKLTVDKSRWQQGNVFSCSVLHEALHSHYTQ | ACEVTHQGLSSPVTKSFNRGEC |
| | KSLSLSPG (SEQ ID NO: 630) | (SEQ ID NO: 666) |
| ARB60 | EVQLVESGGGLVQPGRSLRLSCAASGENFGAFAMHWVRQAPGKGLEWVS | DIQMTQSPSSLSASVGDRVTITCR |
| | AINQGGSEVDYADSVEGRFTISRDNAKNSLYLQMNSLRAEDTAVYYCAR | ASQDISNWLAWYQQKPGKAPKLLI |
| | DPGLPVSAFDIWGLGTMVTVSSASTKGPSVFPLAPSSKSTSGGTAALGC | YDASSLVSGVPSRFSGSGSGTDFT |
| | LVKDYFPEPVTVSWNSGALTSGVHTFPAVLQSSGLYSLSSVVTVPSSSL | LTISSLQPEDVATYYCHQIYDTPP |
| | GTQTYICNVNHKPSNTKVDKKVEPKSCDKTHTCPPCPAPEAAGAPSVFL | TFGGGTKVEIKRTVAAPSVFIFPP |
| | FPPKPKDTLYITREPEVTCVVVDVSHEDPEVKFNWYVDGVEVHNAKTKP | SDEQLKSGTASVVCLLNNFYPREA |
| | REEQYNSTYRVVSVLTVLHQDWLNGKEYKCKVSNKALPAPIEKTISKAK | KVQWKVDNALQSGNSQESVTEQDS |
| | GQPREPQVYTLPPSRDELTKNQVSLTCLVKGFYPSDIAVEWESNGQPEN | KDSTYSLSSTLTLSKADYEKHKVY |
| | NYKTTPPVLDSDGSFFLYSKLTVDKSRWQQGNVFSCSVMHEALHNHYTQ | ACEVTHQGLSSPVTKSFNRGEC |
| | KSLSLSPG (SEQ ID NO: 631) | (SEQ ID NO: 667) |
| ARB61 | EVQLVESGGGLVQPGRSLRLSCAASGENFGAFAMHWVRQAPGKGLEWVS | DIQMTQSPSSLSASVGDRVTITCR |
| | AINQGGSEVDYADSVEGRFTISRDNAKNSLYLQMNSLRAEDTAVYYCAR | ASQDISNWLAWYQQKPGKAPKLLI |
| | DPGLPVSAFDIWGLGTMVTVSSASTKGPSVFPLAPSSKSTSGGTAALGC | YDASSLVSGVPSRFSGSGSGTDFT |
| | LVKDYFPEPVTVSWNSGALTSGVHTFPAVLQSSGLYSLSSVVTVPSSSL | LTISSLQPEDVATYYCHQIYDTPP |
| | GTQTYICNVNHKPSNTKVDKKVEPKSCDKTHTCPPCPAPELLGGDSVFL | TFGGGTKVEIKRTVAAPSVFIFPP |
| | FPPKPKDTLMISRTPEVTCVVVDVSHEDPEVKFNWYVDGVEVHNAKTKP | SDEQLKSGTASVVCLLNNFYPREA |
| | REEQYNSTYRVVSVLTVLHQDWLNGKEYKCKVSNKALPAPIEKTISKAK | KVQWKVDNALQSGNSQESVTEQDS |
| | GQPREPQVYTLPPSRDELTKNQVSLTCLVKGFYPSDIAVEWESNGQPEN | KDSTYSLSSTLTLSKADYEKHKVY |
| | NYKTTPPVLDSDGSFFLYSKLTVDKSRWQQGNVFSCSVMHEALHNHYTQ | ACEVTHQGLSSPVTKSFNRGEC |
| | KSLSLSPG (SEQ ID NO: 632) | (SEQ ID NO: 668) |
| ARB62 | EVQLVESGGGLVQPGRSLRLSCAASGENFGAFAMHWVRQAPGKGLEWVS | DIQMTQSPSSLSASVGDRVTITCR |
| | AINQGGSEVDYADSVEGRFTISRDNAKNSLYLQMNSLRAEDTAVYYCAR | ASQDISNWLAWYQQKPGKAPKLLI |
| | DPGLPVSAFDIWGLGTMVTVSSASTKGPSVFPLAPSSKSTSGGTAALGC | YDASSLVSGVPSRFSGSGSGTDFT |
| | LVKDYFPEPVTVSWNSGALTSGVHTFPAVLQSSGLYSLSSVVTVPSSSL | LTISSLQPEDVATYYCHQIYDTPP |
| | GTQTYICNVNHKPSNTKVDKKVEPKSCDKTHTCPPCPAPELLGGDSVFL | TFGGGTKVEIKRTVAAPSVFIFPP |
| | FPPKPKDTLMISRTPEVTCVVVDVSHEDPEVKFNWYVDGVEVHNAKTKP | SDEQLKSGTASVVCLLNNFYPREA |
| | REEQYNSTYRVVSVLTVLHQDWLNGKEYKCKVSNKALPAPIEKTISKAK | KVQWKVDNALQSGNSQESVTEQDS |
| | GQPREPQVYTLPPSRDELTKNQVSLTCLVKGFYPSDIAVEWESNGQPEN | KDSTYSLSSTLTLSKADYEKHKVY |
| | NYKTTPPVLDSDGSFFLYSKLTVDKSRWQQGNVFSCSVLHEALHSHYTQ | ACEVTHQGLSSPVTKSFNRGEC |
| | KSLSLSPG (SEQ ID NO: 633) | (SEQ ID NO: 669) |
| ARB63 | EVQLVESGGGLVQPGRSLRLSCAASGENFGAFAMHWVRQAPGKGLEWVS | DIQMTQSPSSLSASVGDRVTITCR |
| | AINQGGSEVDYADSVEGRFTISRDNAKNSLYLQMNSLRAEDTAVYYCAR | ASQDISNWLAWYQQKPGKAPKLLI |
| | DPGLPVSAFDIWGLGTMVTVSSASTKGPSVFPLAPSSKSTSGGTAALGC | YDASSLVSGVPSRFSGSGSGTDFT |
| | LVKDYFPEPVTVSWNSGALTSGVHTFPAVLQSSGLYSLSSVVTVPSSSL | LTISSLQPEDVATYYCHQIYDTPP |
| | GTQTYICNVNHKPSNTKVDKKVEPKSCDKTHTCPPCPAPELLGGDSVFL | TFGGGTKVEIKRTVAAPSVFIFPP |
| | FPPKPKDTLYITREPEVTCVVVDVSHEDPEVKFNWYVDGVEVHNAKTKP | SDEQLKSGTASVVCLLNNFYPREA |
| | REEQYNSTYRVVSVLTVLHQDWLNGKEYKCKVSNKALPAPIEKTISKAK | KVQWKVDNALQSGNSQESVTEQDS |
| | GQPREPQVYTLPPSRDELTKNQVSLTCLVKGFYPSDIAVEWESNGQPEN | KDSTYSLSSTLTLSKADYEKHKVY |
| | NYKTTPPVLDSDGSFFLYSKLTVDKSRWQQGNVFSCSVMHEALHNHYTQ | ACEVTHQGLSSPVTKSFNRGEC |
| | KSLSLSPG (SEQ ID NO: 634) | (SEQ ID NO: 670) |
| ARB64 | EVQLVESGGGLVQPGRSLRLSCAASGENFGAFAMHWVRQAPGKGLEWVS | DIQMTQSPSSLSASVGDRVTITCR |
| | AINQGGSEVDYADSVEGRFTISRDNAKNSLYLQMNSLRAEDTAVYYCAR | ASQDISNWLAWYQQKPGKAPKLLI |
| | DPGLPVSAFDIWGLGTMVTVSSASTKGPSVFPLAPSSKSTSGGTAALGC | YDASSLVSGVPSRFSGSGSGTDFT |
| | LVKDYFPEPVTVSWNSGALTSGVHTFPAVLQSSGLYSLSSVVTVPSSSL | LTISSLQPEDVATYYCHQIYDTPP |
| | GTQTYICNVNHKPSNTKVDKKVEPKSCDKTHTCPPCPAPELLGEESVFL | TFGGGTKVEIKRTVAAPSVFIFPP |
| | FPPKPKDTLMISRTPEVTCVVVDVSHEDPEVKFNWYVDGVEVHNAKTKP | SDEQLKSGTASVVCLLNNFYPREA |
| | REEQYNSTYRVVSVLTVLHQDWLNGKEYKCKVSNKALPAPIEKTISKAK | KVQWKVDNALQSGNSQESVTEQDS |
| | GQPREPQVYTLPPSRDELTKNQVSLTCLVKGFYPSDIAVEWESNGQPEN | KDSTYSLSSTLTLSKADYEKHKVY |
| | NYKTTPPVLDSDGSFFLYSKLTVDKSRWQQGNVFSCSVMHEALHNHYTQ | ACEVTHQGLSSPVTKSFNRGEC |
| | KSLSLSPG (SEQ ID NO: 635) | (SEQ ID NO: 671) |
| ARB65 | EVQLVESGGGLVQPGRSLRLSCAASGENFGAFAMHWVRQAPGKGLEWVS | DIQMTQSPSSLSASVGDRVTITCR |
| | AINQGGSEVDYADSVEGRFTISRDNAKNSLYLQMNSLRAEDTAVYYCAR | ASQDISNWLAWYQQKPGKAPKLLI |
| | DPGLPVSAFDIWGLGTMVTVSSASTKGPSVFPLAPSSKSTSGGTAALGC | YDASSLVSGVPSRFSGSGSGTDFT |
| | LVKDYFPEPVTVSWNSGALTSGVHTFPAVLQSSGLYSLSSVVTVPSSSL | LTISSLQPEDVATYYCHQIYDTPP |
| | GTQTYICNVNHKPSNTKVDKKVEPKSCDKTHTCPPCPAPELLGEESVFL | TFGGGTKVEIKRTVAAPSVFIFPP |
| | FPPKPKDTLMISRTPEVTCVVVDVSHEDPEVKFNWYVDGVEVHNAKTKP | SDEQLKSGTASVVCLLNNFYPREA |
| | REEQYNSTYRVVSVLTVLHQDWLNGKEYKCKVSNKALPAPIEKTISKAK | KVQWKVDNALQSGNSQESVTEQDS |
| | GQPREPQVYTLPPSRDELTKNQVSLTCLVKGFYPSDIAVEWESNGQPEN | KDSTYSLSSTLTLSKADYEKHKVY |
| | NYKTTPPVLDSDGSFFLYSKLTVDKSRWQQGNVFSCSVLHEALHSHYTQ | ACEVTHQGLSSPVTKSFNRGEC |
| | KSLSLSPG (SEQ ID NO: 636) | (SEQ ID NO: 672) |
| ARB66 | EVQLVESGGGLVQPGRSLRLSCAASGENFGAFAMHWVRQAPGKGLEWVS | DIQMTQSPSSLSASVGDRVTITCR |
| | AINQGGSEVDYADSVEGRFTISRDNAKNSLYLQMNSLRAEDTAVYYCAR | ASQDISNWLAWYQQKPGKAPKLLI |
| | DPGLPVSAFDIWGLGTMVTVSSASTKGPSVFPLAPSSKSTSGGTAALGC | YDASSLVSGVPSRFSGSGSGTDFT |
| | LVKDYFPEPVTVSWNSGALTSGVHTFPAVLQSSGLYSLSSVVTVPSSSL | LTISSLQPEDVATYYCHQIYDTPP |
| | GTQTYICNVNHKPSNTKVDKKVEPKSCDKTHTCPPCPAPELLGEESVFL | TFGGGTKVEIKRTVAAPSVFIFPP |
| | FPPKPKDTLYITREPEVTCVVVDVSHEDPEVKFNWYVDGVEVHNAKTKP | SDEQLKSGTASVVCLLNNFYPREA |
| | REEQYNSTYRVVSVLTVLHQDWLNGKEYKCKVSNKALPAPIEKTISKAK | KVQWKVDNALQSGNSQESVTEQDS |
| | GQPREPQVYTLPPSRDELTKNQVSLTCLVKGFYPSDIAVEWESNGQPEN | KDSTYSLSSTLTLSKADYEKHKVY |
| | NYKTTPPVLDSDGSFFLYSKLTVDKSRWQQGNVFSCSVMHEALHNHYTQ | ACEVTHQGLSSPVTKSFNRGEC |
| | KSLSLSPG (SEQ ID NO: 637) | (SEQ ID NO: 673) |
| ARB67 | EVQLVESGGGLVQPGRSLRLSCAASGSTLSSYGMHWVRQAPGKGLEWVS | DIQMTQSPSSLSASVGDRVTITCR |
| | AISYDASTIDYADSVEGRFTISRDNAKNSLYLQMNSLRAEDTAVYYCAS | ASQGIGSYLAWYQQKPGKAPKLLI |
| | EYGLAFDQWGQGTLVTVSSASTKGPSVFPLAPSSKSTSGGTAALGCLVK | YEASRLESGVPSRFSGSGSGTDFT |
| | DYFPEPVTVSWNSGALTSGVHTFPAVLQSSGLYSLSSVVTVPSSSLGTQ | LTISSLQPEDVATYYCQQGYNAPI |
| | TYICNVNHKPSNTKVDKKVEPKSCDKTHTCPPCPAPEAAGAPSVFLFPP | TFGGGTKVEIKRTVAAPSVFIFPP |
| | KPKDTLMISRTPEVTCVVVDVSHEDPEVKFNWYVDGVEVHNAKTKPREE | SDEQLKSGTASVVCLLNNFYPREA |
| | QYNSTYRVVSVLTVLHQDWLNGKEYKCKVSNKALPAPIEKTISKAKGQP | KVQWKVDNALQSGNSQESVTEQDS |
| | REPQVYTLPPSRDELTKNQVSLTCLVKGFYPSDIAVEWESNGQPENNYK | KDSTYSLSSTLTLSKADYEKHKVY |
| | TTPPVLDSDGSFFLYSKLTVDKSRWQQGNVFSCSVMHEALHNHYTQKSL | ACEVTHQGLSSPVTKSFNRGEC |
| | SLSPG (SEQ ID NO: 638) | (SEQ ID NO: 674) |
| ARB68 | EVQLVESGGGLVQPGRSLRLSCAASGSTLSSYGMHWVRQAPGKGLEWVS | DIQMTQSPSSLSASVGDRVTITCR |
| | AISYDASTIDYADSVEGRFTISRDNAKNSLYLQMNSLRAEDTAVYYCAS | ASQGIGSYLAWYQQKPGKAPKLLI |
| | EYGLAFDQWGQGTLVTVSSASTKGPSVFPLAPSSKSTSGGTAALGCLVK | YEASRLESGVPSRFSGSGSGTDFT |
| | DYFPEPVTVSWNSGALTSGVHTFPAVLQSSGLYSLSSVVTVPSSSLGTQ | LTISSLQPEDVATYYCQQGYNAPI |
| | TYICNVNHKPSNTKVDKKVEPKSCDKTHTCPPCPAPEAAGAPSVFLFPP | TFGGGTKVEIKRTVAAPSVFIFPP |
| | KPKDTLMISRTPEVTCVVVDVSHEDPEVKFNWYVDGVEVHNAKTKPREE | SDEQLKSGTASVVCLLNNFYPREA |
| | QYNSTYRVVSVLTVLHQDWLNGKEYKCKVSNKALPAPIEKTISKAKGQP | KVQWKVDNALQSGNSQESVTEQDS |
| | REPQVYTLPPSRDELTKNQVSLTCLVKGFYPSDIAVEWESNGQPENNYK | KDSTYSLSSTLTLSKADYEKHKVY |
| | TTPPVLDSDGSFFLYSKLTVDKSRWQQGNVFSCSVLHEALHSHYTQKSL | ACEVTHQGLSSPVTKSFNRGEC |
| | SLSPG (SEQ ID NO: 639) | (SEQ ID NO: 675) |
| ARB69 | EVQLVESGGGLVQPGRSLRLSCAASGSTLSSYGMHWVRQAPGKGLEWVS | DIQMTQSPSSLSASVGDRVTITCR |
| | AISYDASTIDYADSVEGRFTISRDNAKNSLYLQMNSLRAEDTAVYYCAS | ASQGIGSYLAWYQQKPGKAPKLLI |
| | EYGLAFDQWGQGTLVTVSSASTKGPSVFPLAPSSKSTSGGTAALGCLVK | YEASRLESGVPSRFSGSGSGTDFT |
| | DYFPEPVTVSWNSGALTSGVHTFPAVLQSSGLYSLSSVVTVPSSSLGTQ | LTISSLQPEDVATYYCQQGYNAPI |
| | TYICNVNHKPSNTKVDKKVEPKSCDKTHTCPPCPAPEAAGAPSVFLFPP | TFGGGTKVEIKRTVAAPSVFIFPP |
| | KPKDTLYITREPEVTCVVVDVSHEDPEVKFNWYVDGVEVHNAKTKPREE | SDEQLKSGTASVVCLLNNFYPREA |
| | QYNSTYRVVSVLTVLHQDWLNGKEYKCKVSNKALPAPIEKTISKAKGQP | KVQWKVDNALQSGNSQESVTEQDS |
| | REPQVYTLPPSRDELTKNQVSLTCLVKGFYPSDIAVEWESNGQPENNYK | KDSTYSLSSTLTLSKADYEKHKVY |
| | TTPPVLDSDGSFFLYSKLTVDKSRWQQGNVFSCSVMHEALHNHYTQKSL | ACEVTHQGLSSPVTKSFNRGEC |
| | SLSPG (SEQ ID NO: 640) | (SEQ ID NO: 676) |
| ARB70 | EVQLVESGGGLVQPGRSLRLSCAASGSTLSSYGMHWVRQAPGKGLEWVS | DIQMTQSPSSLSASVGDRVTITCR |
| | AISYDASTIDYADSVEGRFTISRDNAKNSLYLQMNSLRAEDTAVYYCAS | ASQGIGSYLAWYQQKPGKAPKLLI |
| | EYGLAFDQWGQGTLVTVSSASTKGPSVFPLAPSSKSTSGGTAALGCLVK | YEASRLESGVPSRFSGSGSGTDFT |
| | DYFPEPVTVSWNSGALTSGVHTFPAVLQSSGLYSLSSVVTVPSSSLGTQ | LTISSLQPEDVATYYCQQGYNAPI |
| | TYICNVNHKPSNTKVDKKVEPKSCDKTHTCPPCPAPELLGGDSVFLFPP | TFGGGTKVEIKRTVAAPSVFIFPP |
| | KPKDTLMISRTPEVTCVVVDVSHEDPEVKFNWYVDGVEVHNAKTKPREE | SDEQLKSGTASVVCLLNNFYPREA |
| | QYNSTYRVVSVLTVLHQDWLNGKEYKCKVSNKALPAPIEKTISKAKGQP | KVQWKVDNALQSGNSQESVTEQDS |
| | REPQVYTLPPSRDELTKNQVSLTCLVKGFYPSDIAVEWESNGQPENNYK | KDSTYSLSSTLTLSKADYEKHKVY |
| | TTPPVLDSDGSFFLYSKLTVDKSRWQQGNVFSCSVMHEALHNHYTQKSL | ACEVTHQGLSSPVTKSFNRGEC |
| | SLSPG (SEQ ID NO: 641) | (SEQ ID NO: 677) |
| ARB71 | EVQLVESGGGLVQPGRSLRLSCAASGSTLSSYGMHWVRQAPGKGLEWVS | DIQMTQSPSSLSASVGDRVTITCR |
| | AISYDASTIDYADSVEGRFTISRDNAKNSLYLQMNSLRAEDTAVYYCAS | ASQGIGSYLAWYQQKPGKAPKLLI |
| | EYGLAFDQWGQGTLVTVSSASTKGPSVFPLAPSSKSTSGGTAALGCLVK | YEASRLESGVPSRFSGSGSGTDFT |
| | DYFPEPVTVSWNSGALTSGVHTFPAVLQSSGLYSLSSVVTVPSSSLGTQ | LTISSLQPEDVATYYCQQGYNAPI |
| | TYICNVNHKPSNTKVDKKVEPKSCDKTHTCPPCPAPELLGGDSVFLFPP | TFGGGTKVEIKRTVAAPSVFIFPP |
| | KPKDTLMISRTPEVTCVVVDVSHEDPEVKFNWYVDGVEVHNAKTKPREE | SDEQLKSGTASVVCLLNNFYPREA |
| | QYNSTYRVVSVLTVLHQDWLNGKEYKCKVSNKALPAPIEKTISKAKGQP | KVQWKVDNALQSGNSQESVTEQDS |
| | REPQVYTLPPSRDELTKNQVSLTCLVKGFYPSDIAVEWESNGQPENNYK | KDSTYSLSSTLTLSKADYEKHKVY |
| | TTPPVLDSDGSFFLYSKLTVDKSRWQQGNVFSCSVLHEALHSHYTQKSL | ACEVTHQGLSSPVTKSFNRGEC |
| | SLSPG (SEQ ID NO: 642) | (SEQ ID NO: 678) |
| ARB72 | EVQLVESGGGLVQPGRSLRLSCAASGSTLSSYGMHWVRQAPGKGLEWVS | DIQMTQSPSSLSASVGDRVTITCR |
| | AISYDASTIDYADSVEGRFTISRDNAKNSLYLQMNSLRAEDTAVYYCAS | ASQGIGSYLAWYQQKPGKAPKLLI |
| | EYGLAFDQWGQGTLVTVSSASTKGPSVFPLAPSSKSTSGGTAALGCLVK | YEASRLESGVPSRFSGSGSGTDFT |
| | DYFPEPVTVSWNSGALTSGVHTFPAVLQSSGLYSLSSVVTVPSSSLGTQ | LTISSLQPEDVATYYCQQGYNAPI |
| | TYICNVNHKPSNTKVDKKVEPKSCDKTHTCPPCPAPELLGGDSVFLFPP | TFGGGTKVEIKRTVAAPSVFIFPP |
| | KPKDTLYITREPEVTCVVVDVSHEDPEVKFNWYVDGVEVHNAKTKPREE | SDEQLKSGTASVVCLLNNFYPREA |
| | QYNSTYRVVSVLTVLHQDWLNGKEYKCKVSNKALPAPIEKTISKAKGQP | KVQWKVDNALQSGNSQESVTEQDS |
| | REPQVYTLPPSRDELTKNQVSLTCLVKGFYPSDIAVEWESNGQPENNYK | KDSTYSLSSTLTLSKADYEKHKVY |
| | TTPPVLDSDGSFFLYSKLTVDKSRWQQGNVFSCSVMHEALHNHYTQKSL | ACEVTHQGLSSPVTKSFNRGEC |
| | SLSPG (SEQ ID NO: 643) | (SEQ ID NO: 679) |
| ARB73 | EVQLVESGGGLVQPGRSLRLSCAASGSTLSSYGMHWVRQAPGKGLEWVS | DIQMTQSPSSLSASVGDRVTITCR |
| | AISYDASTIDYADSVEGRFTISRDNAKNSLYLQMNSLRAEDTAVYYCAS | ASQGIGSYLAWYQQKPGKAPKLLI |
| | EYGLAFDQWGQGTLVTVSSASTKGPSVFPLAPSSKSTSGGTAALGCLVK | YEASRLESGVPSRFSGSGSGTDFT |
| | DYFPEPVTVSWNSGALTSGVHTFPAVLQSSGLYSLSSVVTVPSSSLGTQ | LTISSLQPEDVATYYCQQGYNAPI |
| | TYICNVNHKPSNTKVDKKVEPKSCDKTHTCPPCPAPELLGEESVFLFPP | TFGGGTKVEIKRTVAAPSVFIFPP |
| | KPKDTLMISRTPEVTCVVVDVSHEDPEVKFNWYVDGVEVHNAKTKPREE | SDEQLKSGTASVVCLLNNFYPREA |
| | QYNSTYRVVSVLTVLHQDWLNGKEYKCKVSNKALPAPIEKTISKAKGQP | KVQWKVDNALQSGNSQESVTEQDS |
| | REPQVYTLPPSRDELTKNQVSLTCLVKGFYPSDIAVEWESNGQPENNYK | KDSTYSLSSTLTLSKADYEKHKVY |
| | TTPPVLDSDGSFFLYSKLTVDKSRWQQGNVFSCSVMHEALHNHYTQKSL | ACEVTHQGLSSPVTKSFNRGEC |
| | SLSPG (SEQ ID NO: 644) | (SEQ ID NO: 680) |
| ARB74 | EVQLVESGGGLVQPGRSLRLSCAASGSTLSSYGMHWVRQAPGKGLEWVS | DIQMTQSPSSLSASVGDRVTITCR |
| | AISYDASTIDYADSVEGRFTISRDNAKNSLYLQMNSLRAEDTAVYYCAS | ASQGIGSYLAWYQQKPGKAPKLLI |
| | EYGLAFDQWGQGTLVTVSSASTKGPSVFPLAPSSKSTSGGTAALGCLVK | YEASRLESGVPSRFSGSGSGTDFT |
| | DYFPEPVTVSWNSGALTSGVHTFPAVLQSSGLYSLSSVVTVPSSSLGTQ | LTISSLQPEDVATYYCQQGYNAPI |
| | TYICNVNHKPSNTKVDKKVEPKSCDKTHTCPPCPAPELLGEESVFLFPP | TFGGGTKVEIKRTVAAPSVFIFPP |
| | KPKDTLMISRTPEVTCVVVDVSHEDPEVKFNWYVDGVEVHNAKTKPREE | SDEQLKSGTASVVCLLNNFYPREA |
| | QYNSTYRVVSVLTVLHQDWLNGKEYKCKVSNKALPAPIEKTISKAKGQP | KVQWKVDNALQSGNSQESVTEQDS |
| | REPQVYTLPPSRDELTKNQVSLTCLVKGFYPSDIAVEWESNGQPENNYK | KDSTYSLSSTLTLSKADYEKHKVY |
| | TTPPVLDSDGSFFLYSKLTVDKSRWQQGNVFSCSVLHEALHSHYTQKSL | ACEVTHQGLSSPVTKSFNRGEC |
| | SLSPG (SEQ ID NO: 645) | (SEQ ID NO: 681) |
| ARB75 | EVQLVESGGGLVQPGRSLRLSCAASGSTLSSYGMHWVRQAPGKGLEWVS | DIQMTQSPSSLSASVGDRVTITCR |
| | AISYDASTIDYADSVEGRFTISRDNAKNSLYLQMNSLRAEDTAVYYCAS | ASQGIGSYLAWYQQKPGKAPKLLI |
| | EYGLAFDQWGQGTLVTVSSASTKGPSVFPLAPSSKSTSGGTAALGCLVK | YEASRLESGVPSRFSGSGSGTDFT |
| | DYFPEPVTVSWNSGALTSGVHTFPAVLQSSGLYSLSSVVTVPSSSLGTQ | LTISSLQPEDVATYYCQQGYNAPI |
| | TYICNVNHKPSNTKVDKKVEPKSCDKTHTCPPCPAPELLGEESVFLFPP | TFGGGTKVEIKRTVAAPSVFIFPP |
| | KPKDTLYITREPEVTCVVVDVSHEDPEVKFNWYVDGVEVHNAKTKPREE | SDEQLKSGTASVVCLLNNFYPREA |
| | QYNSTYRVVSVLTVLHQDWLNGKEYKCKVSNKALPAPIEKTISKAKGQP | KVQWKVDNALQSGNSQESVTEQDS |
| | REPQVYTLPPSRDELTKNQVSLTCLVKGFYPSDIAVEWESNGQPENNYK | KDSTYSLSSTLTLSKADYEKHKVY |
| | TTPPVLDSDGSFFLYSKLTVDKSRWQQGNVFSCSVMHEALHNHYTQKSL | ACEVTHQGLSSPVTKSFNRGEC |
| | SLSPG (SEQ ID NO: 646) | (SEQ ID NO: 682) |

In some embodiments, an anti-FcγRIIb antibody, or an antigen-binding fragment thereof, is in a Fab format. In some embodiments, an anti-FcγRIIb antibody, or an antigen-binding fragment thereof, comprises a heavy chain having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, or 646.

In some embodiments, an anti-FcγRIIb antibody, or an antigen-binding fragment thereof, comprises a light chain having an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682.

In some embodiments, an anti-FcγRIIb antibody, or an antigen-binding fragment thereof, comprises a heavy chain having an amino acid sequence of any one of SEQ ID NOs: 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, or 646.

In some embodiments, an anti-FcγRIIb antibody, or an antigen-binding fragment thereof, comprises a light chain having an amino acid sequence of any one of SEQ ID NOs: 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682.

In some embodiments, an anti-FcγRIIb antibody, or an antigen-binding fragment thereof, comprises a heavy chain having an amino acid sequence of SEQ ID NO: 611; and a light chain having an amino acid sequence of SEQ ID NO: 647. In some embodiments, an anti-FcγRIIb antibody, or an antigen-binding fragment thereof, comprises a heavy chain having an amino acid sequence of SEQ ID NO: 612; and a light chain having an amino acid sequence of SEQ ID NO: 648. In some embodiments, an anti-FcγRIIb antibody, or an antigen-binding fragment thereof, comprises a heavy chain having an amino acid sequence of SEQ ID NO: 613; and a light chain having an amino acid sequence of SEQ ID NO: 649. In some embodiments, an anti-FcγRIIb antibody, or an antigen-binding fragment thereof, comprises a heavy chain having an amino acid sequence of SEQ ID NO: 614; and a light chain having an amino acid sequence of SEQ ID NO: 650. In some embodiments, an anti-FcγRIIb antibody, or an antigen-binding fragment thereof, comprises a heavy chain having an amino acid sequence of SEQ ID NO: 615; and a light chain having an amino acid sequence of SEQ ID NO: 651. In some embodiments, an anti-FcγRIIb antibody, or an antigen-binding fragment thereof, comprises a heavy chain having an amino acid sequence of SEQ ID NO: 616; and a light chain having an amino acid sequence of SEQ ID NO: 652. In some embodiments, an anti-FcγRIIb antibody, or an antigen-binding fragment thereof, comprises a heavy chain having an amino acid sequence of SEQ ID NO: 617; and a light chain having an amino acid sequence of SEQ ID NO: 653. In some embodiments, an anti-FcγRIIb antibody, or an antigen-binding fragment thereof, comprises a heavy chain having an amino acid sequence of SEQ ID NO: 618; and a light chain having an amino acid sequence of SEQ ID NO: 654. In some embodiments, an anti-FcγRIIb antibody, or an antigen-binding fragment thereof, comprises a heavy chain having an amino acid sequence of SEQ ID NO: 619; and a light chain having an amino acid sequence of SEQ ID NO: 655. In some embodiments, an anti-FcγRIIb antibody, or an antigen-binding fragment thereof, comprises a heavy chain having an amino acid sequence of SEQ ID NO: 620; and a light chain having an amino acid sequence of SEQ ID NO: 656. In some embodiments, an anti-FcγRIIb antibody, or an antigen-binding fragment thereof, comprises a heavy chain having an amino acid sequence of SEQ ID NO: 621; and a light chain having an amino acid sequence of SEQ ID NO: 657. In some embodiments, an anti-FcγRIIb antibody, or an antigen-binding fragment thereof, comprises a heavy chain having an amino acid sequence of SEQ ID NO: 622; and a light chain having an amino acid sequence of SEQ ID NO: 658. In some embodiments, an anti-FcγRIIb antibody, or an antigen-binding fragment thereof, comprises a heavy chain having an amino acid sequence of SEQ ID NO: 623; and a light chain having an amino acid sequence of SEQ ID NO: 659. In some embodiments, an anti-FcγRIIb antibody, or an antigen-binding fragment thereof, comprises a heavy chain having an amino acid sequence of SEQ ID NO: 624; and a light chain having an amino acid sequence of SEQ ID NO: 660. In some embodiments, an anti-FcγRIIb antibody, or an antigen-binding fragment thereof, comprises a heavy chain having an amino acid sequence of SEQ ID NO: 625; and a light chain having an amino acid sequence of SEQ ID NO: 661. In some embodiments, an anti-FcγRIIb antibody, or an antigen-binding fragment thereof, comprises a heavy chain having an amino acid sequence of SEQ ID NO: 626; and a light chain having an amino acid sequence of SEQ ID NO: 662. In some embodiments, an anti-FcγRIIb antibody, or an antigen-binding fragment thereof, comprises a heavy chain having an amino acid sequence of SEQ ID NO: 627; and a light chain having an amino acid sequence of SEQ ID NO: 663. In some embodiments, an anti-FcγRIIb antibody, or an antigen-binding fragment thereof, comprises a heavy chain having an amino acid sequence of SEQ ID NO: 628; and a light chain having an amino acid sequence of SEQ ID NO: 664. In some embodiments, an anti-FcγRIIb antibody, or an antigen-binding fragment thereof, comprises a heavy chain having an amino acid sequence of SEQ ID NO: 629; and a light chain having an amino acid sequence of SEQ ID NO: 665. In some embodiments, an anti-FcγRIIb antibody, or an antigen-binding fragment thereof, comprises a heavy chain having an amino acid sequence of SEQ ID NO: 630; and a light chain having an amino acid sequence of SEQ ID NO: 666. In some embodiments, an anti-FcγRIIb antibody, or an antigen-binding fragment thereof, comprises a heavy chain having an amino acid sequence of SEQ ID NO: 631; and a light chain having an amino acid sequence of SEQ ID NO: 667. In some embodiments, an anti-FcγRIIb antibody, or an antigen-binding fragment thereof, comprises a heavy chain having an amino acid sequence of SEQ ID NO: 632; and a light chain having an amino acid sequence of SEQ ID NO: 668. In some embodiments, an anti-FcγRIIb antibody, or an antigen-binding fragment thereof, comprises a heavy chain having an amino acid sequence of SEQ ID NO: 633; and a light chain having an amino acid sequence of SEQ ID NO: 669. In some embodiments, an anti-FcγRIIb antibody, or an antigen-binding fragment thereof, comprises a heavy chain having an amino acid sequence of SEQ ID NO: 634; and a light chain having an amino acid sequence of SEQ ID NO: 670. In some embodiments, an anti-FcγRIIb antibody, or an antigen-binding fragment thereof, comprises a heavy chain having an amino acid sequence of SEQ ID NO: 635; and a light chain having an amino acid sequence of SEQ ID NO: 671. In some embodiments, an anti-FcγRIIb antibody, or an antigen-binding fragment thereof, comprises a heavy chain having an amino acid sequence of SEQ ID NO: 636; and a light chain having an amino acid sequence of SEQ ID NO: 672. In some embodiments, an anti-FcγRIIb antibody, or an antigen-binding fragment thereof, comprises a heavy chain having an amino acid sequence of SEQ ID NO: 637; and a light chain having an amino acid sequence of SEQ ID NO: 673. In some embodiments, an anti-FcγRIIb antibody, or an antigen-binding fragment thereof, comprises a heavy chain having an amino acid sequence of SEQ ID NO: 638; and a light chain having an amino acid sequence of SEQ ID NO: 674. In some embodiments, an anti-FcγRIIb antibody, or an antigen-binding fragment thereof, comprises a heavy chain having an amino acid sequence of SEQ ID NO: 639; and a light chain having an amino acid sequence of SEQ ID NO: 675. In some embodiments, an anti-FcγRIIb antibody, or an antigen-binding fragment thereof, comprises a heavy chain having an amino acid sequence of SEQ ID NO: 640; and a light chain having an amino acid sequence of SEQ ID NO: 676. In some embodiments, an anti-FcγRIIb antibody, or an antigen-binding fragment thereof, comprises a heavy chain having an amino acid sequence of SEQ ID NO: 641; and a light chain having an amino acid sequence of SEQ ID NO: 677. In some embodiments, an anti-FcγRIIb antibody, or an antigen-binding fragment thereof, comprises a heavy chain having an amino acid sequence of SEQ ID NO: 642; and a light chain having an amino acid sequence of SEQ ID NO: 678. In some embodiments, an anti-FcγRIIb antibody, or an antigen-binding fragment thereof, comprises a heavy chain having an amino acid sequence of SEQ ID NO: 643; and a light chain having an amino acid sequence of SEQ ID NO: 679. In some embodiments, an anti-FcγRIIb antibody, or an antigen-binding fragment thereof, comprises a heavy chain having an amino acid sequence of SEQ ID NO: 644; and a light chain having an amino acid sequence of SEQ ID NO: 680. In some embodiments, an anti-FcγRIIb antibody, or an antigen-binding fragment thereof, comprises a heavy chain having an amino acid sequence of SEQ ID NO: 645; and a light chain having an amino acid sequence of SEQ ID NO: 681. In some embodiments, an anti-FcγRIIb antibody, or an antigen-binding fragment thereof, comprises a heavy chain having an amino acid sequence of SEQ ID NO: or 646; and a light chain having an amino acid sequence of SEQ ID NO: 682.

In some embodiments, the antibody, or antigen binding fragment thereof, comprises a VH that is at least 90%, 91%, 92%, 93%, 94%, 95%, 96%, 97%, 98%, or 99% identical to the amino acid sequence of SEQ ID NO: 84 and a VL that is at least 90%, 91%, 92%, 93%, 94%, 95%, 96%, 97%, 98%, or 99% identical to the amino acid sequence of SEQ ID NO: 44. In some embodiments, the antibody, or antigen binding fragment thereof, comprises a VH that is at least 90%, 91%, 92%, 93%, 94%, 95%, 96%, 97%, 98%, or 99% identical to the amino acid sequence of SEQ ID NO: 84, provided that the VH comprises a HCDR1 of SEQ ID NO: 606, a HCDR2 of SEQ ID NO: 608, and a HCDR3 of SEQ ID NO: 608, and a VL that is at least 90%, 91%, 92%, 93%, 94%, 95%, 96%, 97%, 98%, or 99% identical to the amino acid sequence of SEQ ID NO: 44, provided that the VL comprises a LCDR1 of SEQ ID NO: 609, a LCDR2 of SEQ ID NO: 610, and a LCDR3 of SEQ ID NO: 586.

In some embodiments, the antibody, or antigen binding fragment thereof, comprises a HC that is at least 90%, 91%, 92%, 93%, 94%, 95%, 96%, 97%, 98%, or 99% identical to the amino acid sequence of SEQ ID NO: 638 and a LC that is at least 90%, 91%, 92%, 93%, 94%, 95%, 96%, 97%, 98%, or 99% identical to the amino acid sequence of SEQ ID NO: 674. In some embodiments, the antibody, or antigen binding fragment thereof, comprises a HC that is at least 90%, 91%, 92%, 93%, 94%, 95%, 96%, 97%, 98%, or 99% identical to the amino acid sequence of SEQ ID NO: 638, provided that the HC comprises a variable heavy chain domain comprising a HCDR1 of SEQ ID NO: 606, a HCDR2 of SEQ ID NO: 608, and a HCDR3 of SEQ ID NO: 608, and a LC that is at least 90%, 91%, 92%, 93%, 94%, 95%, 96%, 97%, 98%, or 99% identical to the amino acid sequence of SEQ ID NO: 674, provided that the LC comprises a light chain variable domain that comprises a LCDR1 of SEQ ID NO: 609, a LCDR2 of SEQ ID NO: 610, and a LCDR3 of SEQ ID NO: 586. In some embodiments, the antibody comprises a VH of SEQ ID NO: 84 and a VL of SEQ ID NO: 44. In some embodiments, the antibody comprises a HC of SEQ ID NO: 638 and a LC of SEQ ID NO: 674.

### Dimeric Molecules

In some embodiments, two (or more) linkers associate, either covalently or non-covalently, e.g., to form a heterodimeric or homodimeric therapeutic compound. In some embodiments, the linker can comprise an Fc polypeptide and two Fc polypeptides associate with one another. In some embodiments of a therapeutic compound comprising two linker regions, the linker regions can self-associate, e.g., as two identical Fc polypeptides. In some embodiments of a therapeutic compound comprising two linker regions, the linker regions are not capable of, or not capable of substantial, self-association, e.g., the two Fc polypeptides can be members of a knob and hole pair. In some embodiments, the polypeptide comprises a first polypeptide and a second polypeptide. In some embodiments, the first polypeptide comprises a knob mutation, and the second polypeptide comprises a hole mutation. In some embodiments, the first polypeptide comprises a hole mutation, and the second polypeptide comprises a knob mutation. In some embodiments, the knob mutation is such as those provided herein. In some embodiments, the hole mutation is such as those provided herein.

In some embodiments, a polypeptide can associate with another polypeptide. In some embodiments, the polypeptide associated with another polypeptide forms a dimer molecule.

In some embodiments, the dimer is a homodimer molecule. In some embodiments, the dimer is a heterodimer molecule.

As used herein, the term “non-covalently conjugated” can mean that a polypeptide is tethered to another polypeptide through a linker. In some embodiments, the linker is a peptide linker. Non-limiting examples of peptide linkers that can be used are known in the art and are provide for herein.

In some embodiments, the polypeptide that is the compound comprises at the N-terminus an antibody comprised of a plurality of Fab on an Fc polypeptide fused to a plurality of scFv on the C-terminus of the Fc polypeptide. In some embodiments, the Fc polypeptide is such as those provided herein. The Fc polypeptides described in this paragraph can be used throughout this application where a Fc polypeptide is referred to as part of the therapeutic compound. The Fc polypeptide can be any one of the Fc polypeptides as provided for herein and further comprise a mutation, or set of mutations, as provided for herein. Thus, as provided for herein, the Fc polypeptide can selectively bind to FcγRIIb over FcγRIIα.

In some embodiments, the antibody comprised of a plurality of Fab fused to an Fc polypeptide can be an anti-PD-1 antibody, or an antigen-binding fragment thereof, an anti-LAG-3, or an anti-CTLA4 antibody (or any other antibody that binds to an inhibitory receptor). In some embodiments, the plurality of scFv polypeptides fused to the C-terminus could be the anti-FcγRII antibody. In some embodiments, the polypeptide comprises two antibodies linked separately to two separate anti-FcγRII antibodies. In some embodiments, the Fab bind to PD-1 or LAG-3. In some embodiments, one antibody binds to PD-1 and the other binds to LAG-3.

In some embodiments, the FcγRII binding effector domain is an anti-FcγRIIb antibody, such as those provided for herein. In some embodiments, the anti-FcγRIIb antibody, or an antigen-binding fragment thereof, provided for herein is selective for FcγRIIb over the FcγRIIα-R131 isoform or the FcγRIIα-H131 isoform. Without being bound to any particular theory, these FcγRIIb binding effector domain can be used to help down regulate or inhibit an immune response. In some embodiments, n some embodiments, the anti-FcγRIIb antibody, or an antigen-binding fragment thereof, provided for herein is selective for FcγRIIα-R131 isoform or the FcγRIIα-H131 isoform over FcγRIIb.

In some embodiments, an Fc dimer molecule comprises a first Fc polypeptide and a second Fc polypeptide. In some embodiments, a dimer molecule comprises a first polypeptide and a second polypeptide, wherein the first polypeptide and the second polypeptide comprise different amino acid sequence. In some embodiments, the dimer molecule is a homodimer molecule. In some embodiments, the dimer molecule is a heterodimer molecule. In some embodiments, the dimer molecule comprises a pair of a first polypeptide and a second polypeptide, and wherein the first polypeptide and the second polypeptide comprise different amino acid sequences. In some embodiments, the dimer molecule is a Fc polypeptide comprising a first polypeptide and a second polypeptide. In some embodiments, the first polypeptide is a first Fc polypeptide. In some embodiments, the second polypeptide is a second Fc polypeptide. In some embodiments, the first Fc polypeptide and the second Fc polypeptide are not the same. In some embodiments, the first Fc polypeptide and the second Fc polypeptide are different. In some embodiments, the first polypeptide is such a those provided herein, e.g., SEQ ID NOs: 417, 418, 419, 420, 421, 422, 423, 424, 425, 426, 427, 428, 429, 430, 431, 432, 434, 435, 436, 437, 438, 439, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 543, 544, 545, 546, 683, 684, 685, 686, 687, 688, 689, and 690. In some embodiments, the second polypeptide is such a those provided herein, e.g., SEQ ID NOs: 417, 418, 419, 420, 421, 422, 423, 424, 425, 426, 427, 428, 429, 430, 431, 432, 434, 435, 436, 437, 438, 439, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 543, 544, 545, 546, 683, 684, 685, 686, 687, 688, 689, and 690. In some embodiments, the Fc polypeptide comprises the first Fc polypeptide comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to anyone of SEQ ID NOs: 417, 418, 419, 420, 421, 422, 423, 424, 425, 426, 427, 428, 429, 430, 431, 432, 434, 435, 436, 437, 438, 439, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 543, 544, 545, 546, 683, 684, 685, 686, 687, 688, 689, and 690. In some embodiments, the second polypeptide is such a those provided herein, e.g., SEQ ID NOs: 417, 418, 419, 420, 421, 422, 423, 424, 425, 426, 427, 428, 429, 430, 431, 432, 434, 435, 436, 437, 438, 439, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 546, 683, 684, 685, 686, 687, 688, 689, and 690; and the second Fc polypeptide comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to anyone of SEQ ID NOs: 417, 418, 419, 420, 421, 422, 423, 424, 425, 426, 427, 428, 429, 430, 431, 432, 434, 435, 436, 437, 438, 439, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 543, 544, 545, 546, 683, 684, 685, 686, 687, 688, 689, and 690. In some embodiments, the Fc polypeptide comprises the first Fc polypeptide comprising an amino acid sequence selected from any one of SEQ ID NOs: 417, 418, 419, 420, 421, 422, 423, 424, 425, 426, 427, 428, 429, 430, 431, 432, 434, 435, 436, 437, 438, 439, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 543, 544, 545, 546, 683, 684, 685, 686, 687, 688, 689, and 690; and the second Fc polypeptide comprising an amino acid sequence selected from any one of SEQ ID NOs: 417, 418, 419, 420, 421, 422, 423, 424, 425, 426, 427, 428, 429, 430, 431, 432, 434, 435, 436, 437, 438, 439, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 543, 544, 545, 546, 683, 684, 685, 686, 687, 688, 689, and 690. In some embodiments, the Fc dimer comprises the first polypeptide and the second polypeptide as provided in Table 8 below.

In some embodiments, the Fc dimer comprises the first Fc polypeptide and the second Fc polypeptide as provided in VFC-86 through VFC-9685. In some embodiments, the Fc dimer comprises VFC-86 through VFC-9685. In some embodiments, the Fc dimer consists of VFC-86 through VFC-9685.

As discussed herein the different domains, molecules, or polypeptides can be linked together with a linker domain or region. Any linker region described herein can be used as a linker. Linkers can be for example, glycine/serine linkers. In some embodiments, the linker can comprise one or more repeats of GGGGS (SEQ ID NO: 1). In some embodiments, the linker comprises 1, 2, 3, 4, or 5 repeats. In some embodiments, the linker comprises GGGGSGGGGS (SEQ ID NO: 2). In some embodiments, the linker comprises GGGGSGGGGSGGGGS (SEQ ID NO: 3). In some embodiments, the linker comprises: GGGGS (SEQ ID NO: 1), (GGGGS) 3 (SEQ ID NO: 3), (GGGGS)ₙ (n=1, 2, 3, 4) (SEQ ID NO: 1-4), (Gly)₈ (SEQ ID NO: 5), (Gly)₆ (SEQ ID NO: 6). (EAAAK)₃ (SEQ ID NO: 7), (EAAK)ₙ (n=1-3) (SEQ ID NO: 8-10), A(EAAAK)₄ALEA(EAAAK)₄A (SEQ ID NO: 11), or AEAAAKEAAAKA (SEQ ID NO: 12). These linkers can be used in any of the compounds or compositions provided herein. These peptide linkers are non-limiting examples and other peptide linkers can also be used.

In some embodiments, the polypeptide forms a dimer. In some embodiments, the dimer is a homodimer. In some embodiments, the dimer is a heterodimer.

Non-limiting exemplary configurations of therapeutic compounds comprise the following (e.g., in N-terminus to C-terminus order):
R1-Linker Region A-R2
R3-Linker Region B-R4,
   wherein,
R1, R2, R3, and R4, each independently comprises an effector binding/modulating moiety, e.g., anti-PD1 antibody, anti-LAG3 antibody, anti-CTLA4 antibody, anti-FcγRIIb antibody; or is absent;
Linker Region A and Linker Region B comprise moieties that can associate with one another, e.g., Linker A and Linker Region B, each comprises an Fc polypeptide provided that an effector binding/modulating moiety and a specific targeting moiety are present. Furthermore, Linker A and Linker Region B, each comprise an Fc polypeptide that is selective for FcγRIIb. In some embodiments, the Fc polypeptide that is selective for FcγRIIb has increased selectivity for FcγRIIb. In some embodiments, the Fc polypeptide that is selective for FcγRIIb has increased affinity for FcγRIIb. In some embodiments, the Fc polypeptide that is selective for FcγRIIb has increased selectivity and affinity for FcγRIIb. In some embodiments, the Fc polypeptide that is selective for FcγRIIb has increased selectivity for FcγRIIb over FcγRIIα. In some embodiments, the Fc polypeptide that is selective for FcγRIIb has increased affinity for FcγRIIb over FcγRIIα. In some embodiments, the Fc polypeptide that is selective for FcγRIIb has increased selectivity and affinity for FcγRIIb over FcγRIIα. In some embodiments, Linker Region A and Linker Region B are both absent.

In some embodiments, the dimer comprises the formula of:
R1-Linker Region A-R2
R3-Linker Region B—R4,
wherein,
   R1, R2, R3, and R4, each independently comprises an effector binding/modulating moiety, e.g., anti-PD1 antibody, anti-LAG3 antibody, anti-CTLA4 antibody, anti-FcγRIIb antibody; or is absent; and
   Linker Region A and Linker Region B, each independently comprises an Fc polypeptide provided that the effector binding/modulating moieties are present, and wherein the Fc polypeptide selectively binds to FcγRIIb.

In some embodiments:
R1 comprises an effector binding/modulating moiety, e.g., anti-PD-1 antibody, or an antigen-binding fragment thereof, anti-LAG3 antibody, anti-CTLA4 antibody, or anti-FcγRIIb antibody, or is absent;
R2 comprises an effector binding/modulating moiety, e.g., anti-PD-1 antibody, or an antigen-binding fragment thereof, anti-LAG3 antibody, anti-CTLA4 antibody, or anti-FcγRIIb antibody;
R3 comprises an effector binding/modulating moiety, e.g., anti-PD-1 antibody, or an antigen-binding fragment thereof, anti-LAG3 antibody, anti-CTLA4 antibody, or anti-FcγRIIb antibody, or is absent;
R4 comprises an effector binding/modulating moiety, e.g., anti-PD-1 antibody, or an antigen-binding fragment thereof, anti-LAG3 antibody, anti-CTLA4 antibody, or anti-FcγRIIb antibody; and
Linker Region A and Linker Region B comprise moieties that can associate with one another, e.g., Linker A and Linker Region B, each comprises an Fc polypeptide, provided that one of R1 or R3 is present and one of R2 or R4 is present. Furthermore, Linker A and Linker Region B, each comprise an Fc polypeptide that is selective for FcγRIIb. In some embodiments, the Fc polypeptide that is selective for FcγRIIb has increased selectivity for FcγRIIb. In some embodiments, the Fc polypeptide that is selective for FcγRIIb has increased affinity for FcγRIIb. In some embodiments, the Fc polypeptide that is selective for FcγRIIb has increased selectivity and affinity for FcγRIIb. In some embodiments, the Fc polypeptide that is selective for FcγRIIb has increased selectivity for FcγRIIb over FcγRIIα. In some embodiments, the Fc polypeptide that is selective for FcγRIIb has increased affinity for FcγRIIb over FcγRIIα. In some embodiments, the Fc polypeptide that is selective for FcγRIIb has increased selectivity and affinity for FcγRIIb over FcγRIIα.

Non-limiting exemplary configurations of therapeutic compounds comprise the following (e.g., in N-terminus to C-terminus order):
R1-Linker Region A-R2
R3-Linker Region B—R4,
   wherein,
R1, R2, R3, and R4, each independently comprises an effector binding/modulating moiety, e.g., anti-PD1 antibody, anti-LAG3 antibody, anti-CTLA4 antibody, anti-FcγRIIb antibody; or is absent;
Linker Region A and Linker Region B comprise moieties that can associate with one another, e.g., Linker A and Linker Region B, each comprises an Fc polypeptide provided that an effector binding/modulating moiety and a specific targeting moiety are present. Furthermore, Linker A and Linker Region B, each comprise an Fc polypeptide that is selective for FcγRIIb. In some embodiments, the Fc polypeptide that is selective for FcγRIIb has increased selectivity for FcγRIIb. In some embodiments, the Fc polypeptide that is selective for FcγRIIb has increased affinity for FcγRIIb. In some embodiments, the Fc polypeptide that is selective for FcγRIIb has increased selectivity and affinity for FcγRIIb. In some embodiments, the Fc polypeptide that is selective for FcγRIIb has increased selectivity for FcγRIIb over FcγRIIα. In some embodiments, the Fc polypeptide that is selective for FcγRIIb has increased affinity for FcγRIIb over FcγRIIα. In some embodiments, the Fc polypeptide that is selective for FcγRIIb has increased selectivity and affinity for FcγRIIb over FcγRIIα. In some embodiments, Linker Region A and Linker Region B are both absent.

In some embodiments, the dimer comprises the formula of:
R1-Linker Region A-R2
R3-Linker Region B—R4,
wherein,
   R1, R2, R3, and R4, each independently comprises an effector binding/modulating moiety, e.g., anti-PD1 antibody, anti-LAG3 antibody, anti-CTLA4 antibody, anti-FcγRIIb antibody; or is absent; and
   Linker Region A and Linker Region B, each independently comprises an Fc polypeptide provided that the effector binding/modulating moieties are present, and wherein the Fc polypeptide selectively binds to FcγRIIb.

In some embodiments:
R1 comprises an effector binding/modulating moiety, e.g., anti-PD-1 antibody, or an antigen-binding fragment thereof, anti-LAG3 antibody, anti-CTLA4 antibody, or anti-FcγRIIb antibody, or is absent;
R2 comprises an effector binding/modulating moiety, e.g., anti-PD-1 antibody, or an antigen-binding fragment thereof, anti-LAG3 antibody, anti-CTLA4 antibody, or anti-FcγRIIb antibody;
R3 comprises an effector binding/modulating moiety, e.g., anti-PD-1 antibody, or an antigen-binding fragment thereof, anti-LAG3 antibody, anti-CTLA4 antibody, or anti-FcγRIIb antibody, or is absent;
R4 comprises an effector binding/modulating moiety, e.g., anti-PD-1 antibody, or an antigen-binding fragment thereof, anti-LAG3 antibody, anti-CTLA4 antibody, or anti-FcγRIIb antibody; and
Linker Region A and Linker Region B comprise moieties that can associate with one another, e.g., Linker A and Linker Region B, each comprises an Fc polypeptide, provided that one of R1 or R3 is present and one of R2 or R4 is present. Furthermore, Linker A and Linker Region B, each comprise an Fc polypeptide that is selective for FcγRIIb. In some embodiments, the Fc polypeptide that is selective for FcγRIIb has increased selectivity for FcγRIIb. In some embodiments, the Fc polypeptide that is selective for FcγRIIb has increased affinity for FcγRIIb. In some embodiments, the Fc polypeptide that is selective for FcγRIIb has increased selectivity and affinity for FcγRIIb. In some embodiments, the Fc polypeptide that is selective for FcγRIIb has increased selectivity for FcγRIIb over FcγRIIα. In some embodiments, the Fc polypeptide that is selective for FcγRIIb has increased affinity for FcγRIIb over FcγRIIα. In some embodiments, the Fc polypeptide that is selective for FcγRIIb has increased selectivity and affinity for FcγRIIb over FcγRIIα.

In some embodiments, the effector domain is an anti-PD-1 antibody, or an antigen-binding fragment thereof, such as those provided for herein.

In some embodiments, non-limiting example of a molecule comprising an anti-FcγRIIb antibody, or an antigen-binding fragment thereof, include those set forth in Table 9 below. In some embodiments, the signal peptide is optional, and thus, non-limiting examples of molecules optionally comprising the signal peptide may comprise the signal peptide. In some embodiments, non-limiting examples of molecules optionally comprising the signal peptide may not comprise the signal peptide. In some embodiments, the molecule comprising an anti-FcγRIIb antibody, or an antigen-binding fragment thereof, comprises a Kappa constant region (Ck) amino acid sequence. In some embodiments, the Ck amino acid sequence is as provided in SEQ ID NO: 415.

| | Polypeptide 1 | | | | | | | Polypeptide 2 | |
|---|---|---|---|---|---|---|---|---|---|
| | | | | C- | scFv VH | | scFv VL | VL | |
| Molecule | Signal | VH (anti- | | terminal | (anti- | scFv | (anti- | (anti- | |
| ID | Peptide | PD1) | Fc | linker | FcγRIIb) | Linker | FcyRIIb) | PD1) | Ck |
| TA1 | MGWS | QVQLVQ | ASTKGPSVFPLAP | GGGGS | EVQLV | GGGGS | DIVMT | DIQMT | RTVAAPSV |
| | CIILFL | SGAEVK | SSKSTSGGTAALG | GGGGS | ESGGGL | GGGGS | QSPLSL | QSPSSL | FIFPPSDEQ |
| | VATA | KPGASV | CLVKDYFPEPVTV | GGGGS | VQPGG | GGGGS | PVTPGE | SASVG | LKSGTASV |
| | TGVH | KVSCKV | SWNSGALTSGVH | (SEQ ID | SLRLSC | GGGGS | PASISC | DRVTIT | VCLLNNF |
| | S (SEQ | SGYSLS | TFPAVLQSSGLYS | NO: 3) | AASAFT | (SEQ ID | RSSQSL | CQASQ | YPREAKV |
| | ID NO: | KYDMS | LSSVVTVPSSSLG | | FSAHS | NO: 4) | VHGSG | SPNNL | QWKVDNA |
| | 588) | WVRQA | TQTYICNVNHKPS | | MSWVR | | YTFLH | LAWY | LQSGNSQE |
| | | PGKGLE | NTKVDKKVEPKS | | QAPGK | | WYLQK | QQKPG | SVTEQDSK |
| | | WMGIIY | CDKTHTCPPCPAP | | GLELV | | PGQSPQ | KAPKL | DSTYSLSS |
| | | TSGYTD | EAAGAPSVFLFPP | | ASISPS | | LLIYDA | LIYGAS | TLTLSKAD |
| | | YAQKFQ | KPKDTLMISRTPE | | GSVTY | | VTRAT | DLPSG | YEKHKVY |
| | | GRVTMT | VTCVVVDVSHED | | YPDSV | | GVPDRF | VPSRFS | ACEVTHQ |
| | | EDTSTD | PEVKFNWYVDGV | | KGRFTI | | SGSGSG | GSGSG | GLSSPVTK |
| | | TAYMEL | EVHNAKTKPREE | | SRDNA | | TDFTLK | TDFTL | SFNRGEC |
| | | SSLRSED | QYNSTYRVVSVL | | KNSLYL | | ISRVEA | TISSLQ | (SEQ ID |
| | | TAVYYC | TVLHQDWLNGKE | | QMNSL | | EDVGV | PEDFA | NO: 415) |
| | | ATGNPY | YKCKVSNKALPA | | RAEDT | | YYCMQ | TYYCQ | |
| | | YTNGFN | PIEKTISKAKGQPR | | AVYYC | | TTEFPY | NNYYV | |
| | | SWGQGT | EPQVYTLPPSRDE | | ARDSGS | | TFGGGT | GPVSY | |
| | | LVTVSS | LTKNQVSLTCLV | | YRDAF | | KVEIK | AFGGG | |
| | | (SEQ ID | KGFYPSDIAVEWE | | DIWGQ | | (SEQ ID | TKVEI | |
| | | NO: 514) | SNGQPENNYKTTP | | GTMVT | | NO: 18) | K (SEQ | |
| | | | PVLDSDGSFFLYS | | VSS | | | ID NO: | |
| | | | KLTVDKSRWQQG | | (SEQ ID | | | 515) | |
| | | | NVFSCSVMHEAL | | NO: 54) | | | | |
| | | | HNHYTQKSLSLSP | | | | | | |
| | | | G (SEQ ID NO: 513) | | | | | | |
| TA2 | MGWS | QVQLVQ | ASTKGPSVFPLAP | GGGGS | EVQLV | GGGGS | DIVMT | DIQMT | RTVAAPSV |
| | CIILFL | SGAEVK | SSKSTSGGTAALG | GGGGS | ESGGGL | GGGGS | QSPLSL | QSPSSL | FIFPPSDEQ |
| | VATA | KPGASV | CLVKDYFPEPVTV | GGGGS | VQPGG | GGGGS | PVTPGE | SASVG | LKSGTASV |
| | TGVH | KVSCKV | SWNSGALTSGVH | (SEQ ID | SLRLSC | GGGGS | PASISC | DRVTIT | VCLLNNF |
| | S (SEQ | SGYSLS | TFPAVLQSSGLYS | NO: 3) | AASGFT | (SEQ ID | RSSQSL | CQASQ | YPREAKV |
| | ID NO: | KYDMS | LSSVVTVPSSSLG | | FAGHA | NO: 4) | LHSTGY | SPNNL | QWKVDNA |
| | 588) | WVRQA | TQTYICNVNHKPS | | MSWVR | | NFLHW | LAWY | LQSGNSQE |
| | | PGKGLE | NTKVDKKVEPKS | | QAPGK | | YLQKP | QQKPG | SVTEQDSK |
| | | WMGIIY | CDKTHTCPPCPAP | | GLELV | | GQSPQL | KAPKL | DSTYSLSS |
| | | TSGYTD | EAAGAPSVFLFPP | | ASISPS | | LIYDAS | LIYGAS | TLTLSKAD |
| | | YAQKFQ | KPKDTLMISRTPE | | GSTTYY | | KRAPG | DLPSG | YEKHKVY |
| | | GRVTMT | VTCVVVDVSHED | | PDSVK | | VPDRFS | VPSRFS | ACEVTHQ |
| | | EDTSTD | PEVKFNWYVDGV | | GRFTIS | | GSGSGT | GSGSG | GLSSPVTK |
| | | TAYMEL | EVHNAKTKPREE | | RDNAK | | DFTLKI | TDFTL | SFNRGEC |
| | | SSLRSED | QYNSTYRVVSVL | | NSLYLQ | | SRVEAE | TISSLQ | (SEQ ID |
| | | TAVYYC | TVLHQDWLNGKE | | MNSLR | | DVGVY | PEDFA | NO: 415) |
| | | ATGNPY | YKCKVSNKALPA | | AEDTA | | YCMQT | TYYCQ | |
| | | YTNGFN | PIEKTISKAKGQPR | | VYYCA | | VELPFT | NNYYV | |
| | | SWGQGT | EPQVYTLPPSRDE | | RDGDSS | | FGGGT | GPVSY | |
| | | LVTVSS | LTKNQVSLTCLV | | DAFDI | | KVEIK | AFGGG | |
| | | (SEQ ID | KGFYPSDIAVEWE | | WGQGT | | (SEQ ID | TKVEI | |
| | | NO: 514) | SNGQPENNYKTTP | | MVTVS | | NO: 19) | K (SEQ | |
| | | | PVLDSDGSFFLYS | | S (SEQ | | | ID NO: | |
| | | | KLTVDKSRWQQG | | ID NO: | | | 515) | |
| | | | NVFSCSVMHEAL | | 55) | | | | |
| | | | HNHYTQKSLSLSP | | | | | | |
| | | | G (SEQ ID NO: 513) | | | | | | |
| TA3 | MGWS | QVQLVQ | ASTKGPSVFPLAP | GGGGS | QVQLQ | GGGGS | EIVMTQ | DIQMT | RTVAAPSV |
| | CIILFL | SGAEVK | SSKSTSGGTAALG | GGGGS | ESGPGL | GGGGS | SPATLS | QSPSSL | FIFPPSDEQ |
| | VATA | KPGASV | CLVKDYFPEPVTV | GGGGS | VKPSQT | GGGGS | LSPGER | SASVG | LKSGTASV |
| | TGVH | KVSCKV | SWNSGALTSGVH | (SEQ ID | LSLTCT | GGGGS | ATLSCR | DRVTIT | VCLLNNF |
| | S (SEQ | SGYSLS | TFPAVLQSSGLYS | NO: 3) | VSGASI | (SEQ ID | ASQSV | CQASQ | YPREAKV |
| | ID NO: | KYDMS | LSSVVTVPSSSLG | | STIDYS | NO: 4) | DRHLA | SPNNL | QWKVDNA |
| | 588) | WVRQA | TQTYICNVNHKPS | | WSWIR | | WYQQK | LAWY | LQSGNSQE |
| | | PGKGLE | NTKVDKKVEPKS | | QPPGK | | PGQAPR | QQKPG | SVTEQDSK |
| | | WMGIIY | CDKTHTCPPCPAP | | GLEWIG | | LLIYDV | KAPKL | DSTYSLSS |
| | | TSGYTD | EAAGAPSVFLFPP | | YIDESG | | SNRAPG | LIYGAS | TLTLSKAD |
| | | YAQKFQ | KPKDTLMISRTPE | | RIDYNP | | IPARFS | DLPSG | YEKHKVY |
| | | GRVTMT | VTCVVVDVSHED | | SLKSRV | | GSGSGT | VPSRFS | ACEVTHQ |
| | | EDTSTD | PEVKFNWYVDGV | | TMSVD | | DFTLTI | GSGSG | GLSSPVTK |
| | | TAYMEL | EVHNAKTKPREE | | TSKNQF | | SSLEPE | TDFTL | SFNRGEC |
| | | SSLRSED | QYNSTYRVVSVL | | SLKVNS | | DFAVY | TISSLQ | (SEQ ID |
| | | TAVYYC | TVLHQDWLNGKE | | VTAAD | | YCQQY | PEDFA | NO: 415) |
| | | ATGNPY | YKCKVSNKALPA | | TAVYY | | GWPSIT | TYYCQ | |
| | | YTNGFN | PIEKTISKAKGQPR | | CAREG | | FGGGT | NNYYV | |
| | | SWGQGT | EPQVYTLPPSRDE | | QWGQF | | KVEIK | GPVSY | |
| | | LVTVSS | LTKNQVSLTCLV | | DYWGQ | | (SEQ ID | AFGGG | |
| | | (SEQ ID | KGFYPSDIAVEWE | | GTLVT | | NO: 20) | TKVEI | |
| | | NO: 514) | SNGQPENNYKTTP | | VSS | | | K (SEQ | |
| | | | PVLDSDGSFFLYS | | (SEQ ID | | | ID NO: | |
| | | | KLTVDKSRWQQG | | NO: 56) | | | 515) | |
| | | | NVFSCSVMHEAL | | | | | | |
| | | | HNHYTQKSLSLSP | | | | | | |
| | | | G (SEQ ID NO: 513) | | | | | | |
| TA4 | MGWS | QVQLVQ | ASTKGPSVFPLAP | GGGGS | EVQLV | GGGGS | DIVMT | DIQMT | RTVAAPSV |
| | CIILFL | SGAEVK | SSKSTSGGTAALG | GGGGS | ESGGGL | GGGGS | QSPLSL | QSPSSL | FIFPPSDEQ |
| | VATA | KPGASV | CLVKDYFPEPVTV | GGGGS | VQPGG | GGGGS | PVTPGE | SASVG | LKSGTASV |
| | TGVH | KVSCKV | SWNSGALTSGVH | (SEQ ID | SLRLSC | GGGGS | PASISC | DRVTIT | VCLLNNF |
| | S (SEQ | SGYSLS | TFPAVLQSSGLYS | NO: 3) | AASGFT | (SEQ ID | RSSQSL | CQASQ | YPREAKV |
| | ID NO: | KYDMS | LSSVVTVPSSSLG | | FSNHA | NO: 4) | LSSYGY | SPNNL | QWKVDNA |
| | 588) | WVRQA | TQTYICNVNHKPS | | MSWVR | | HNLHW | LAWY | LQSGNSQE |
| | | PGKGLE | NTKVDKKVEPKS | | QAPGK | | YLQKP | QQKPG | SVTEQDSK |
| | | WMGIIY | CDKTHTCPPCPAP | | GLELV | | GQSPQL | KAPKL | DSTYSLSS |
| | | TSGYTD | EAAGAPSVFLFPP | | ASINPS | | LIYDAY | LIYGAS | TLTLSKAD |
| | | YAQKFQ | KPKDTLMISRTPE | | GSVTY | | VRATG | DLPSG | YEKHKVY |
| | | GRVTMT | VTCVVVDVSHED | | YPDSV | | VPDRFS | VPSRFS | ACEVTHQ |
| | | EDTSTD | PEVKFNWYVDGV | | KGRFTI | | GSGSGT | GSGSG | GLSSPVTK |
| | | TAYMEL | EVHNAKTKPREE | | SRDNA | | DFTLKI | TDFTL | SFNRGEC |
| | | SSLRSED | QYNSTYRVVSVL | | KNSLYL | | SRVEAE | TISSLQ | (SEQ ID |
| | | TAVYYC | TVLHQDWLNGKE | | QMNSL | | DVGVY | PEDFA | NO: 415) |
| | | ATGNPY | YKCKVSNKALPA | | RAEDT | | YCMQS | TYYCQ | |
| | | YTNGFN | PIEKTISKAKGQPR | | AVYYC | | KELPYT | NNYYV | |
| | | SWGQGT | EPQVYTLPPSRDE | | ARDGD | | FGGGT | GPVSY | |
| | | LVTVSS | LTKNQVSLTCLV | | YGDYL | | KVEIK | AFGGG | |
| | | (SEQ ID | KGFYPSDIAVEWE | | DYWGQ | | (SEQ ID | TKVEI | |
| | | NO: 514) | SNGQPENNYKTTP | | GTLVT | | NO: 21) | K (SEQ | |
| | | | PVLDSDGSFFLYS | | VSS | | | ID NO: | |
| | | | KLTVDKSRWQQG | | (SEQ ID | | | 515) | |
| | | | NVFSCSVMHEAL | | NO: 57) | | | | |
| | | | HNHYTQKSLSLSP | | | | | | |
| | | | G (SEQ ID NO: 513) | | | | | | |
| TA5 | MGWS | QVQLVQ | ASTKGPSVFPLAP | GGGGS | QVQLV | GGGGS | DIQMT | DIQMT | RTVAAPSV |
| | CIILFL | SGAEVK | SSKSTSGGTAALG | GGGGS | QSGAE | GGGGS | QSPSSV | QSPSSL | FIFPPSDEQ |
| | VATA | KPGASV | CLVKDYFPEPVTV | GGGGS | VKKPG | GGGGS | SASVG | SASVG | LKSGTASV |
| | TGVH | KVSCKV | SWNSGALTSGVH | (SEQ ID | ASVKV | GGGGS | DRVTIT | DRVTIT | VCLLNNF |
| | S (SEQ | SGYSLS | TFPAVLQSSGLYS | NO: 3) | SCKVSG | (SEQ ID | CRASQ | CQASQ | YPREAKV |
| | ID NO: | KYDMS | LSSVVTVPSSSLG | | DTFTSN | NO: 4) | DIGSNL | SPNNL | QWKVDNA |
| | 588) | WVRQA | TQTYICNVNHKPS | | AIHWV | | AWYQQ | LAWY | LQSGNSQE |
| | | PGKGLE | NTKVDKKVEPKS | | RQAPG | | KPGKA | QQKPG | SVTEQDSK |
| | | WMGIIY | CDKTHTCPPCPAP | | KGLEW | | PKLLIY | KAPKL | DSTYSLSS |
| | | TSGYTD | EAAGAPSVFLFPP | | MGGIV | | SGSTLQ | LIYGAS | TLTLSKAD |
| | | YAQKFQ | KPKDTLMISRTPE | | AGSGH | | SGVPSR | DLPSG | YEKHKVY |
| | | GRVTMT | VTCVVVDVSHED | | TIYAQK | | FSGSGS | VPSRFS | ACEVTHQ |
| | | EDTSTD | PEVKFNWYVDGV | | FQGRV | | GTDFTL | GSGSG | GLSSPVTK |
| | | TAYMEL | EVHNAKTKPREE | | TMTED | | TISSLQP | TDFTL | SFNRGEC |
| | | SSLRSED | QYNSTYRVVSVL | | TSTDTA | | EDFAN | TISSLQ | (SEQ ID |
| | | TAVYYC | TVLHQDWLNGKE | | YMELSS | | YYCQQ | PEDFA | NO: 415) |
| | | ATGNPY | YKCKVSNKALPA | | LKSEDT | | TSSSPP | TYYCQ | |
| | | YTNGFN | PIEKTISKAKGQPR | | AVYYC | | YTFGG | NNYYV | |
| | | SWGQGT | EPQVYTLPPSRDE | | AREGA | | GTKVEI | GPVSY | |
| | | LVTVSS | LTKNQVSLTCLV | | EYNGF | | K (SEQ | AFGGG | |
| | | (SEQ ID | KGFYPSDIAVEWE | | DPWGQ | | ID NO: | TKVEI | |
| | | NO: 514) | SNGQPENNYKTTP | | GTLVT | | 22) | K (SEQ | |
| | | | PVLDSDGSFFLYS | | VSS | | | ID NO: | |
| | | | KLTVDKSRWQQG | | (SEQ ID | | | 515) | |
| | | | NVFSCSVMHEAL | | NO: 58) | | | | |
| | | | HNHYTQKSLSLSP | | | | | | |
| | | | G (SEQ ID NO: 513) | | | | | | |
| TA6 | MGWS | QVQLVQ | ASTKGPSVFPLAP | GGGGS | QVQLV | GGGGS | DIQMT | DIQMT | RTVAAPSV |
| | CIILFL | SGAEVK | SSKSTSGGTAALG | GGGGS | QSGAE | GGGGS | QSPSSV | QSPSSL | FIFPPSDEQ |
| | VATA | KPGASV | CLVKDYFPEPVTV | GGGGS | VKKPG | GGGGS | SASVG | SASVG | LKSGTASV |
| | TGVH | KVSCKV | SWNSGALTSGVH | (SEQ ID | ASVKV | GGGGS | DRVTIT | DRVTIT | VCLLNNF |
| | S (SEQ | SGYSLS | TFPAVLQSSGLYS | NO: 3) | SCKVSG | (SEQ ID | CRASQ | CQASQ | YPREAKV |
| | ID NO: | KYDMS | LSSVVTVPSSSLG | | FTFTSS | NO: 4) | NIGNW | SPNNL | QWKVDNA |
| | 588) | WVRQA | TQTYICNVNHKPS | | VIHWV | | LAWYQ | LAWY | LQSGNSQE |
| | | PGKGLE | NTKVDKKVEPKS | | RQAPG | | QKPGK | QQKPG | SVTEQDSK |
| | | WMGIIY | CDKTHTCPPCPAP | | KGLEW | | APKLLI | KAPKL | DSTYSLSS |
| | | TSGYTD | EAAGAPSVFLFPP | | MGGISP | | YAASN | LIYGAS | TLTLSKAD |
| | | YAQKFQ | KPKDTLMISRTPE | | RGESTI | | LQRGV | DLPSG | YEKHKVY |
| | | GRVTMT | VTCVVVDVSHED | | YAQKF | | PSRFSG | VPSRFS | ACEVTHQ |
| | | EDTSTD | PEVKFNWYVDGV | | QGRVT | | SGSGTD | GSGSG | GLSSPVTK |
| | | TAYMEL | EVHNAKTKPREE | | MTEDT | | FTLTISS | TDFTL | SFNRGEC |
| | | SSLRSED | QYNSTYRVVSVL | | STDTAY | | LQPEDF | TISSLQ | (SEQ ID |
| | | TAVYYC | TVLHQDWLNGKE | | MELSSL | | ANYYC | PEDFA | NO: 415) |
| | | ATGNPY | YKCKVSNKALPA | | KSEDTA | | QQYHNI | TYYCQ | |
| | | YTNGFN | PIEKTISKAKGQPR | | VYYCA | | PITFGG | NNYYV | |
| | | SWGQGT | EPQVYTLPPSRDE | | REGAST | | GTKVEI | GPVSY | |
| | | LVTVSS | LTKNQVSLTCLV | | GAFDI | | K (SEQ | AFGGG | |
| | | (SEQ ID | KGFYPSDIAVEWE | | WGQGT | | ID NO: | TKVEI | |
| | | NO: 514) | SNGQPENNYKTTP | | MVTVS | | 23) | K (SEQ | |
| | | | PVLDSDGSFFLYS | | S (SEQ | | | ID NO: | |
| | | | KLTVDKSRWQQG | | ID NO: | | | 515) | |
| | | | NVFSCSVMHEAL | | 59) | | | | |
| | | | HNHYTQKSLSLSP | | | | | | |
| | | | G (SEQ ID NO: 513) | | | | | | |
| TA7 | MGWS | QVQLVQ | ASTKGPSVFPLAP | GGGGS | QVQLV | GGGGS | DIQMT | DIQMT | RTVAAPSV |
| | CIILFL | SGAEVK | SSKSTSGGTAALG | GGGGS | QSGAE | GGGGS | QSPSSV | QSPSSL | FIFPPSDEQ |
| | VATA | KPGASV | CLVKDYFPEPVTV | GGGGS | VKKPG | GGGGS | SASVG | SASVG | LKSGTASV |
| | TGVH | KVSCKV | SWNSGALTSGVH | (SEQ ID | ASVKV | GGGGS | DRVTIT | DRVTIT | VCLLNNF |
| | S (SEQ | SGYSLS | TFPAVLQSSGLYS | NO: 3) | SCKVSG | (SEQ ID | CRASQ | CQASQ | YPREAKV |
| | ID NO: | KYDMS | LSSVVTVPSSSLG | | YTLTG | NO: 4) | GIGTYL | SPNNL | QWKVDNA |
| | 588) | WVRQA | TQTYICNVNHKPS | | NAIHW | | AWYQQ | LAWY | LQSGNSQE |
| | | PGKGLE | NTKVDKKVEPKS | | VRQAP | | KPGKA | QQKPG | SVTEQDSK |
| | | WMGIIY | CDKTHTCPPCPAP | | GKGLE | | PKLLIY | KAPKL | DSTYSLSS |
| | | TSGYTD | EAAGAPSVFLFPP | | WMGGII | | DASILG | LIYGAS | TLTLSKAD |
| | | YAQKFQ | KPKDTLMISRTPE | | PSIGTAI | | SGVPSR | DLPSG | YEKHKVY |
| | | GRVTMT | VTCVVVDVSHED | | YAQKF | | FSGSGS | VPSRFS | ACEVTHQ |
| | | EDTSTD | PEVKFNWYVDGV | | QGRVT | | GTDFTL | GSGSG | GLSSPVTK |
| | | TAYMEL | EVHNAKTKPREE | | MTEDT | | TISSLQP | TDFTL | SFNRGEC |
| | | SSLRSED | QYNSTYRVVSVL | | STDTAY | | EDFAN | TISSLQ | (SEQ ID |
| | | TAVYYC | TVLHQDWLNGKE | | MELSSL | | YYCQH | PEDFA | NO: 415) |
| | | ATGNPY | YKCKVSNKALPA | | KSEDTA | | YGTSPP | TYYCQ | |
| | | YTNGFN | PIEKTISKAKGQPR | | VYYCA | | TFGGGT | NNYYV | |
| | | SWGQGT | EPQVYTLPPSRDE | | KDRSDI | | KVEIK | GPVSY | |
| | | LVTVSS | LTKNQVSLTCLV | | GDIFDY | | (SEQ ID | AFGGG | |
| | | (SEQ ID | KGFYPSDIAVEWE | | WGQGT | | NO: 24) | TKVEI | |
| | | NO: 514) | SNGQPENNYKTTP | | LVTVSS | | | K (SEQ | |
| | | | PVLDSDGSFFLYS | | (SEQ ID | | | ID NO: | |
| | | | KLTVDKSRWQQG | | NO: 60) | | | 515) | |
| | | | NVFSCSVMHEAL | | | | | | |
| | | | HNHYTQKSLSLSP | | | | | | |
| | | | G (SEQ ID NO: 513) | | | | | | |
| TA8 | MGWS | QVQLVQ | ASTKGPSVFPLAP | GGGGS | EVQLV | GGGGS | DIVMT | DIQMT | RTVAAPSV |
| | CIILFL | SGAEVK | SSKSTSGGTAALG | GGGGS | ESGGGL | GGGGS | QSPLSL | QSPSSL | FIFPPSDEQ |
| | VATA | KPGASV | CLVKDYFPEPVTV | GGGGS | VQPGG | GGGGS | PVTPGE | SASVG | LKSGTASV |
| | TGVH | KVSCKV | SWNSGALTSGVH | (SEQ ID | SLRLSC | GGGGS | PASISC | DRVTIT | VCLLNNF |
| | S (SEQ | SGYSLS | TFPAVLQSSGLYS | NO: 3) | AASGFT | (SEQ ID | RSSQSL | CQASQ | YPREAKV |
| | ID NO: | KYDMS | LSSVVTVPSSSLG | | FTTHSM | NO: 4) | LHSTGY | SPNNL | QWKVDNA |
| | 588) | WVRQA | TQTYICNVNHKPS | | SWVRQ | | NFLHW | LAWY | LQSGNSQE |
| | | PGKGLE | NTKVDKKVEPKS | | APGKG | | YLQKP | QQKPG | SVTEQDSK |
| | | WMGIIY | CDKTHTCPPCPAP | | LELVAS | | GQSPQL | KAPKL | DSTYSLSS |
| | | TSGYTD | EAAGAPSVFLFPP | | INPAGSI | | LIYDTF | LIYGAS | TLTLSKAD |
| | | YAQKFQ | KPKDTLMISRTPE | | TYYPDS | | HRATG | DLPSG | YEKHKVY |
| | | GRVTMT | VTCVVVDVSHED | | VKGRF | | VPDRFS | VPSRFS | ACEVTHQ |
| | | EDTSTD | PEVKFNWYVDGV | | TISRDN | | GSGSGT | GSGSG | GLSSPVTK |
| | | TAYMEL | EVHNAKTKPREE | | AKNSL | | DFTLKI | TDFTL | SFNRGEC |
| | | SSLRSED | QYNSTYRVVSVL | | YLQMN | | SRVEAE | TISSLQ | (SEQ ID |
| | | TAVYYC | TVLHQDWLNGKE | | SLRAED | | DVGVY | PEDFA | NO: 415) |
| | | ATGNPY | YKCKVSNKALPA | | TAVYY | | YCMQS | TYYCQ | |
| | | YTNGFN | PIEKTISKAKGQPR | | CARDN | | LQPRFT | NNYYV | |
| | | SWGQGT | EPQVYTLPPSRDE | | NYGYG | | FGGGT | GPVSY | |
| | | LVTVSS | LTKNQVSLTCLV | | DHFDY | | KVEIK | AFGGG | |
| | | (SEQ ID | KGFYPSDIAVEWE | | WGQGT | | (SEQ ID | TKVEI | |
| | | NO: 514) | SNGQPENNYKTTP | | LVTVSS | | NO: 25) | K (SEQ | |
| | | | PVLDSDGSFFLYS | | (SEQ ID | | | ID NO: | |
| | | | KLTVDKSRWQQG | | NO: 61) | | | 515) | |
| | | | NVFSCSVMHEAL | | | | | | |
| | | | HNHYTQKSLSLSP | | | | | | |
| | | | G (SEQ ID NO: 513) | | | | | | |
| TA9 | MGWS | QVQLVQ | ASTKGPSVFPLAP | GGGGS | EVQLV | GGGGS | DIVMT | DIQMT | RTVAAPSV |
| | CIILFL | SGAEVK | SSKSTSGGTAALG | GGGGS | ESGGGL | GGGGS | QSPLSL | QSPSSL | FIFPPSDEQ |
| | VATA | KPGASV | CLVKDYFPEPVTV | GGGGS | VQPGG | GGGGS | PVTPGE | SASVG | LKSGTASV |
| | TGVH | KVSCKV | SWNSGALTSGVH | (SEQ ID | SLRLSC | GGGGS | PASISC | DRVTIT | VCLLNNF |
| | S (SEQ | SGYSLS | TFPAVLQSSGLYS | NO: 3) | AASGFT | (SEQ ID | RSSQSL | CQASQ | YPREAKV |
| | ID NO: | KYDMS | LSSVVTVPSSSLG | | FGDHV | NO: 4) | LHSTGY | SPNNL | QWKVDNA |
| | 588) | WVRQA | TQTYICNVNHKPS | | MSWVR | | NYLHW | LAWY | LQSGNSQE |
| | | PGKGLE | NTKVDKKVEPKS | | QAPGK | | YLQKP | QQKPG | SVTEQDSK |
| | | WMGIIY | CDKTHTCPPCPAP | | GLELV | | GQSPQL | KAPKL | DSTYSLSS |
| | | TSGYTD | EAAGAPSVFLFPP | | ASISPS | | LIYDTS | LIYGAS | TLTLSKAD |
| | | YAQKFQ | KPKDTLMISRTPE | | GDVTY | | TRASG | DLPSG | YEKHKVY |
| | | GRVTMT | VTCVVVDVSHED | | YPDSV | | VPDRFS | VPSRFS | ACEVTHQ |
| | | EDTSTD | PEVKFNWYVDGV | | KGRFTI | | GSGSGT | GSGSG | GLSSPVTK |
| | | TAYMEL | EVHNAKTKPREE | | SRDNA | | DFTLKI | TDFTL | SFNRGEC |
| | | SSLRSED | QYNSTYRVVSVL | | KNSLYL | | SRVEAE | TISSLQ | (SEQ ID |
| | | TAVYYC | TVLHQDWLNGKE | | QMNSL | | DVGVY | PEDFA | NO: 415) |
| | | ATGNPY | YKCKVSNKALPA | | RAEDT | | YCMQT | TYYCQ | |
| | | YTNGFN | PIEKTISKAKGQPR | | AVYYC | | FHLPFT | NNYYV | |
| | | SWGQGT | EPQVYTLPPSRDE | | TTDGDS | | FGGGT | GPVSY | |
| | | LVTVSS | LTKNQVSLTCLV | | DAFDI | | KVEIK | AFGGG | |
| | | (SEQ ID | KGFYPSDIAVEWE | | WGQGT | | (SEQ ID | TKVEI | |
| | | NO: 514) | SNGQPENNYKTTP | | MVTVS | | NO: 26) | K (SEQ | |
| | | | PVLDSDGSFFLYS | | S (SEQ | | | ID NO: | |
| | | | KLTVDKSRWQQG | | ID NO: | | | 515) | |
| | | | NVFSCSVMHEAL | | 62) | | | | |
| | | | HNHYTQKSLSLSP | | | | | | |
| | | | G (SEQ ID NO: 513) | | | | | | |
| TA10 | MGWS | QVQLVQ | ASTKGPSVFPLAP | GGGGS | EVQLV | GGGGS | DIVMT | DIQMT | RTVAAPSV |
| | CIILFL | SGAEVK | SSKSTSGGTAALG | GGGGS | ESGGGL | GGGGS | QSPLSL | QSPSSL | FIFPPSDEQ |
| | VATA | KPGASV | CLVKDYFPEPVTV | GGGGS | VQPGG | GGGGS | PVTPGE | SASVG | LKSGTASV |
| | TGVH | KVSCKV | SWNSGALTSGVH | (SEQ ID | SLRLSC | GGGGS | PASISC | DRVTIT | VCLLNNF |
| | S (SEQ | SGYSLS | TFPAVLQSSGLYS | NO: 3) | AASGLT | (SEQ ID | RSSQSL | CQASQ | YPREAKV |
| | ID NO: | KYDMS | LSSVVTVPSSSLG | | FSNHA | NO: 4) | LHSSGY | SPNNL | QWKVDNA |
| | 588) | WVRQA | TQTYICNVNHKPS | | MSWVR | | NYLHW | LAWY | LQSGNSQE |
| | | PGKGLE | NTKVDKKVEPKS | | QAPGK | | YLQKP | QQKPG | SVTEQDSK |
| | | WMGIIY | CDKTHTCPPCPAP | | GLELV | | GQSPQL | KAPKL | DSTYSLSS |
| | | TSGYTD | EAAGAPSVFLFPP | | ASINPS | | LIYDTT | LIYGAS | TLTLSKAD |
| | | YAQKFQ | KPKDTLMISRTPE | | GSVTY | | NRATG | DLPSG | YEKHKVY |
| | | GRVTMT | VTCVVVDVSHED | | YPDSV | | VPDRFS | VPSRFS | ACEVTHQ |
| | | EDTSTD | PEVKFNWYVDGV | | KGRFTI | | GSGSGT | GSGSG | GLSSPVTK |
| | | TAYMEL | EVHNAKTKPREE | | SRDNA | | DFTLKI | TDFTL | SFNRGEC |
| | | SSLRSED | QYNSTYRVVSVL | | KNSLYL | | SRVEAE | TISSLQ | (SEQ ID |
| | | TAVYYC | TVLHQDWLNGKE | | QMNSL | | DVGVY | PEDFA | NO: 415) |
| | | ATGNPY | YKCKVSNKALPA | | RAEDT | | YCMQT | TYYCQ | |
| | | YTNGFN | PIEKTISKAKGQPR | | AVYYC | | TQLPYT | NNYYV | |
| | | SWGQGT | EPQVYTLPPSRDE | | TADDY | | FGGGT | GPVSY | |
| | | LVTVSS | LTKNQVSLTCLV | | GDYLD | | KVEIK | AFGGG | |
| | | (SEQ ID | KGFYPSDIAVEWE | | YWGQG | | (SEQ ID | TKVEI | |
| | | NO: 514) | SNGQPENNYKTTP | | TLVTVS | | NO: 27) | K (SEQ | |
| | | | PVLDSDGSFFLYS | | S (SEQ | | | ID NO: | |
| | | | KLTVDKSRWQQG | | ID NO: | | | 515) | |
| | | | NVFSCSVMHEAL | | 63) | | | | |
| | | | HNHYTQKSLSLSP | | | | | | |
| | | | G (SEQ ID NO: 513) | | | | | | |
| TA11 | MGWS | QVQLVQ | ASTKGPSVFPLAP | GGGGS | QVQLV | GGGGS | DIQMT | DIQMT | RTVAAPSV |
| | CIILFL | SGAEVK | SSKSTSGGTAALG | GGGGS | QSGAE | GGGGS | QSPSSV | QSPSSL | FIFPPSDEQ |
| | VATA | KPGASV | CLVKDYFPEPVTV | GGGGS | VKKPG | GGGGS | SASVG | SASVG | LKSGTASV |
| | TGVH | KVSCKV | SWNSGALTSGVH | (SEQ ID | ASVKV | GGGGS | DRVTIT | DRVTIT | VCLLNNF |
| | S (SEQ | SGYSLS | TFPAVLQSSGLYS | NO: 3) | SCKVSG | (SEQ ID | CRASQ | CQASQ | YPREAKV |
| | ID NO: | KYDMS | LSSVVTVPSSSLG | | YTFSNY | NO: 4) | GIGRSL | SPNNL | QWKVDNA |
| | 588) | WVRQA | TQTYICNVNHKPS | | VIHWV | | AWYQQ | LAWY | LQSGNSQE |
| | | PGKGLE | NTKVDKKVEPKS | | RQAPG | | KPGKA | QQKPG | SVTEQDSK |
| | | WMGIIY | CDKTHTCPPCPAP | | KGLEW | | PKLLIY | KAPKL | DSTYSLSS |
| | | TSGYTD | EAAGAPSVFLFPP | | MGGIV | | KDTER | LIYGAS | TLTLSKAD |
| | | YAQKFQ | KPKDTLMISRTPE | | AGSGH | | ASGVPS | DLPSG | YEKHKVY |
| | | GRVTMT | VTCVVVDVSHED | | TIYAQK | | RFSGSG | VPSRFS | ACEVTHQ |
| | | EDTSTD | PEVKFNWYVDGV | | FQGRV | | SGTDFT | GSGSG | GLSSPVTK |
| | | TAYMEL | EVHNAKTKPREE | | TMTED | | LTISSL | TDFTL | SFNRGEC |
| | | SSLRSED | QYNSTYRVVSVL | | TSTDTA | | QPEDFA | TISSLQ | (SEQ ID |
| | | TAVYYC | TVLHQDWLNGKE | | YMELSS | | NYYCQ | PEDFA | NO: 415) |
| | | ATGNPY | YKCKVSNKALPA | | LKSEDT | | QVHTFP | TYYCQ | |
| | | YTNGFN | PIEKTISKAKGQPR | | AVYYC | | PTFGGG | NNYYV | |
| | | SWGQGT | EPQVYTLPPSRDE | | ATESAA | | TKVEIK | GPVSY | |
| | | LVTVSS | LTKNQVSLTCLV | | GNWFD | | (SEQ ID | AFGGG | |
| | | (SEQ ID | KGFYPSDIAVEWE | | PWGQG | | NO: 28) | TKVEI | |
| | | NO: 514) | SNGQPENNYKTTP | | TLVTVS | | | K (SEQ | |
| | | | PVLDSDGSFFLYS | | S (SEQ | | | ID NO: | |
| | | | KLTVDKSRWQQG | | ID NO: | | | 515) | |
| | | | NVFSCSVMHEAL | | 64) | | | | |
| | | | HNHYTQKSLSLSP | | | | | | |
| | | | G (SEQ ID NO: 513) | | | | | | |
| TA12 | MGWS | QVQLVQ | ASTKGPSVFPLAP | GGGGS | EVQLV | GGGGS | DIVMT | DIQMT | RTVAAPSV |
| | CIILFL | SGAEVK | SSKSTSGGTAALG | GGGGS | ESGGGL | GGGGS | QSPLSL | QSPSSL | FIFPPSDEQ |
| | VATA | KPGASV | CLVKDYFPEPVTV | GGGGS | VQPGG | GGGGS | PVTPGE | SASVG | LKSGTASV |
| | TGVH | KVSCKV | SWNSGALTSGVH | (SEQ ID | SLRLSC | GGGGS | PASISC | DRVTIT | VCLLNNF |
| | S (SEQ | SGYSLS | TFPAVLQSSGLYS | NO: 3) | AASGFT | (SEQ ID | RSSQSL | CQASQ | YPREAKV |
| | ID NO: | KYDMS | LSSVVTVPSSSLG | | FSDHT | NO: 4) | LHVTG | SPNNL | QWKVDNA |
| | 588) | WVRQA | TQTYICNVNHKPS | | MSWVR | | YNYLH | LAWY | LOSGNSQE |
| | | PGKGLE | NTKVDKKVEPKS | | QAPGK | | WYLQK | QQKPG | SVTEQDSK |
| | | WMGIIY | CDKTHTCPPCPAP | | GLELV | | PGQSPQ | KAPKL | DSTYSLSS |
| | | TSGYTD | EAAGAPSVFLFPP | | ASINPS | | LLIYET | LIYGAS | TLTLSKAD |
| | | YAQKFQ | KPKDTLMISRTPE | | GSVTY | | SKRAPG | DLPSG | YEKHKVY |
| | | GRVTMT | VTCVVVDVSHED | | YPDSV | | VPDRFS | VPSRFS | ACEVTHQ |
| | | EDTSTD | PEVKFNWYVDGV | | KGRFTI | | GSGSGT | GSGSG | GLSSPVTK |
| | | TAYMEL | EVHNAKTKPREE | | SRDNA | | DFTLKI | TDFTL | SFNRGEC |
| | | SSLRSED | QYNSTYRVVSVL | | KNSLYL | | SRVEAE | TISSLQ | (SEQ ID |
| | | TAVYYC | TVLHQDWLNGKE | | QMNSL | | DVGVY | PEDFA | NO: 415) |
| | | ATGNPY | YKCKVSNKALPA | | RAEDT | | YCMQT | TYYCQ | |
| | | YTNGFN | PIEKTISKAKGQPR | | AVYYC | | THWPS | NNYYV | |
| | | SWGQGT | EPQVYTLPPSRDE | | ARDGG | | TFGGGT | GPVSY | |
| | | LVTVSS | LTKNQVSLTCLV | | YGDYF | | KVEIK | AFGGG | |
| | | (SEQ ID | KGFYPSDIAVEWE | | DYWGQ | | (SEQ ID | TKVEI | |
| | | NO: 514) | SNGQPENNYKTTP | | GTLVT | | NO: 29) | K (SEQ | |
| | | | PVLDSDGSFFLYS | | VSS | | | ID NO: | |
| | | | KLTVDKSRWQQG | | (SEQ ID | | | 515) | |
| | | | NVFSCSVMHEAL | | NO: 65) | | | | |
| | | | HNHYTQKSLSLSP | | | | | | |
| | | | G (SEQ ID NO: 513) | | | | | | |
| TA13 | MGWS | QVQLVQ | ASTKGPSVFPLAP | GGGGS | QVQLV | GGGGS | DIQMT | DIQMT | RTVAAPSV |
| | CIILFL | SGAEVK | SSKSTSGGTAALG | GGGGS | QSGAE | GGGGS | QSPSSV | QSPSSL | FIFPPSDEQ |
| | VATA | KPGASV | CLVKDYFPEPVTV | GGGGS | VKKPG | GGGGS | SASVG | SASVG | LKSGTASV |
| | TGVH | KVSCKV | SWNSGALTSGVH | (SEQ ID | ASVKV | GGGGS | DRVTIT | DRVTIT | VCLLNNF |
| | S (SEQ | SGYSLS | TFPAVLQSSGLYS | NO: 3) | SCKVSG | (SEQ ID | CRASQS | CQASQ | YPREAKV |
| | ID NO: | KYDMS | LSSVVTVPSSSLG | | TPLPAT | NO: 4) | IGTNLA | SPNNL | QWKVDNA |
| | 588) | WVRQA | TQTYICNVNHKPS | | IHWVR | | WYQQK | LAWY | LQSGNSQE |
| | | PGKGLE | NTKVDKKVEPKS | | QAPGK | | PGKAP | QQKPG | SVTEQDSK |
| | | WMGIIY | CDKTHTCPPCPAP | | GLEWM | | KLLIYD | KAPKL | DSTYSLSS |
| | | TSGYTD | EAAGAPSVFLFPP | | GGINPS | | ASKRPT | LIYGAS | TLTLSKAD |
| | | YAQKFQ | KPKDTLMISRTPE | | GATIYA | | GVPSRF | DLPSG | YEKHKVY |
| | | GRVTMT | VTCVVVDVSHED | | QKFQG | | SGSGSG | VPSRFS | ACEVTHQ |
| | | EDTSTD | PEVKFNWYVDGV | | RVTMT | | TDFTLT | GSGSG | GLSSPVTK |
| | | TAYMEL | EVHNAKTKPREE | | EDTSTD | | ISSLQPE | TDFTL | SFNRGEC |
| | | SSLRSED | QYNSTYRVVSVL | | TAYME | | DFANY | TISSLQ | (SEQ ID |
| | | TAVYYC | TVLHQDWLNGKE | | LSSLKS | | YCQQG | PEDFA | NO: 415) |
| | | ATGNPY | YKCKVSNKALPA | | EDTAV | | YDIPLT | TYYCQ | |
| | | YTNGFN | PIEKTISKAKGQPR | | YYCAK | | FGGGT | NNYYV | |
| | | SWGQGT | EPQVYTLPPSRDE | | DSDVA | | KVEIK | GPVSY | |
| | | LVTVSS | LTKNQVSLTCLV | | AAGSFF | | (SEQ ID | AFGGG | |
| | | (SEQ ID | KGFYPSDIAVEWE | | DYWGQ | | NO: 30) | TKVEI | |
| | | NO: 514) | SNGQPENNYKTTP | | GTLVT | | | K (SEQ | |
| | | | PVLDSDGSFFLYS | | VSS | | | ID NO: | |
| | | | KLTVDKSRWQQG | | (SEQ ID | | | 515) | |
| | | | NVFSCSVMHEAL | | NO: 66) | | | | |
| | | | HNHYTQKSLSLSP | | | | | | |
| | | | G (SEQ ID NO: 513) | | | | | | |
| TA14 | MGWS | QVQLVQ | ASTKGPSVFPLAP | GGGGS | QVQLV | GGGGS | DIQMT | DIQMT | RTVAAPSV |
| | CIILFL | SGAEVK | SSKSTSGGTAALG | GGGGS | QSGAE | GGGGS | QSPSSV | QSPSSL | FIFPPSDEQ |
| | VATA | KPGASV | CLVKDYFPEPVTV | GGGGS | VKKPG | GGGGS | SASVG | SASVG | LKSGTASV |
| | TGVH | KVSCKV | SWNSGALTSGVH | (SEQ ID | ASVKV | GGGGS | DRVTIT | DRVTIT | VCLLNNF |
| | S (SEQ | SGYSLS | TFPAVLQSSGLYS | NO: 3) | SCKVSG | (SEQ ID | CRASQ | CQASQ | YPREAKV |
| | ID NO: | KYDMS | LSSVVTVPSSSLG | | YTFRN | NO: 4) | NIGDRL | SPNNL | QWKVDNA |
| | 588) | WVRQA | TQTYICNVNHKPS | | YAIHW | | AWYQQ | LAWY | LQSGNSQE |
| | | PGKGLE | NTKVDKKVEPKS | | VRQAP | | KPGKA | QQKPG | SVTEQDSK |
| | | WMGIIY | CDKTHTCPPCPAP | | GKGLE | | PKLLIY | KAPKL | DSTYSLSS |
| | | TSGYTD | EAAGAPSVFLFPP | | WMGGI | | QDRNR | LIYGAS | TLTLSKAD |
| | | YAQKFQ | KPKDTLMISRTPE | | SPSGST | | PSGVPS | DLPSG | YEKHKVY |
| | | GRVTMT | VTCVVVDVSHED | | TIYAQK | | RFSGSG | VPSRFS | ACEVTHQ |
| | | EDTSTD | PEVKFNWYVDGV | | FQGRV | | SGTDFT | GSGSG | GLSSPVTK |
| | | TAYMEL | EVHNAKTKPREE | | TMTED | | LTISSL | TDFTL | SFNRGEC |
| | | SSLRSED | QYNSTYRVVSVL | | TSTDTA | | QPEDFA | TISSLQ | (SEQ ID |
| | | TAVYYC | TVLHQDWLNGKE | | YMELSS | | NYYCQ | PEDFA | NO: 415) |
| | | ATGNPY | YKCKVSNKALPA | | LKSEDT | | QYATSP | TYYCQ | |
| | | YTNGFN | PIEKTISKAKGQPR | | AVYYC | | FTFGGG | NNYYV | |
| | | SWGQGT | EPQVYTLPPSRDE | | ARESAE | | TKVEIK | GPVSY | |
| | | LVTVSS | LTKNQVSLTCLV | | NYGDY | | (SEQ ID | AFGGG | |
| | | (SEQ ID | KGFYPSDIAVEWE | | LDYWG | | NO: 31) | TKVEI | |
| | | NO: 514) | SNGQPENNYKTTP | | QGTLV | | | K (SEQ | |
| | | | PVLDSDGSFFLYS | | TVSS | | | ID NO: | |
| | | | KLTVDKSRWQQG | | (SEQ ID | | | 515) | |
| | | | NVFSCSVMHEAL | | NO: 67) | | | | |
| | | | HNHYTQKSLSLSP | | | | | | |
| | | | G (SEQ ID NO: 513) | | | | | | |
| TA15 | MGWS | QVQLVQ | ASTKGPSVFPLAP | GGGGS | QVQLV | GGGGS | DIQMT | DIQMT | RTVAAPSV |
| | CIILFL | SGAEVK | SSKSTSGGTAALG | GGGGS | QSGAE | GGGGS | QSPSSV | QSPSSL | FIFPPSDEQ |
| | VATA | KPGASV | CLVKDYFPEPVTV | GGGGS | VKKPG | GGGGS | SASVG | SASVG | LKSGTASV |
| | TGVH | KVSCKV | SWNSGALTSGVH | (SEQ ID | ASVKV | GGGGS | DRVTIT | DRVTIT | VCLLNNF |
| | S (SEQ | SGYSLS | TFPAVLQSSGLYS | NO: 3) | SCKVSG | (SEQ ID | CRASQ | CQASQ | YPREAKV |
| | ID NO: | KYDMS | LSSVVTVPSSSLG | | IDLTTS | NO: 4) | NIDTYL | SPNNL | QWKVDNA |
| | 588) | WVRQA | TQTYICNVNHKPS | | AIHWV | | AWYQQ | LAWY | LQSGNSQE |
| | | PGKGLE | NTKVDKKVEPKS | | RQAPG | | KPGKA | QQKPG | SVTEQDSK |
| | | WMGIIY | CDKTHTCPPCPAP | | KGLEW | | PKLLIY | KAPKL | DSTYSLSS |
| | | TSGYTD | EAAGAPSVFLFPP | | MGGIAI | | EGTKRP | LIYGAS | TLTLSKAD |
| | | YAQKFQ | KPKDTLMISRTPE | | GSGHTI | | SGVPSR | DLPSG | YEKHKVY |
| | | GRVTMT | VTCVVVDVSHED | | YAQKF | | FSGSGS | VPSRFS | ACEVTHQ |
| | | EDTSTD | PEVKFNWYVDGV | | QGRVT | | GTDFTL | GSGSG | GLSSPVTK |
| | | TAYMEL | EVHNAKTKPREE | | MTEDT | | TISSLQP | TDFTL | SFNRGEC |
| | | SSLRSED | QYNSTYRVVSVL | | STDTAY | | EDFAN | TISSLQ | (SEQ ID |
| | | TAVYYC | TVLHQDWLNGKE | | MELSSL | | YYCQQ | PEDFA | NO: 415) |
| | | ATGNPY | YKCKVSNKALPA | | KSEDTA | | WDNLP | TYYCQ | |
| | | YTNGFN | PIEKTISKAKGQPR | | VYYCA | | LTFGGG | NNYYV | |
| | | SWGQGT | EPQVYTLPPSRDE | | RDGNF | | TKVEIK | GPVSY | |
| | | LVTVSS | LTKNQVSLTCLV | | GDYIEY | | (SEQ ID | AFGGG | |
| | | (SEQ ID | KGFYPSDIAVEWE | | WGQGT | | NO: 32) | TKVEI | |
| | | NO: 514) | SNGQPENNYKTTP | | LVTVSS | | | K (SEQ | |
| | | | PVLDSDGSFFLYS | | (SEQ ID | | | ID NO: | |
| | | | KLTVDKSRWQQG | | NO : 68) | | | 515) | |
| | | | NVFSCSVMHEAL | | | | | | |
| | | | HNHYTQKSLSLSP | | | | | | |
| | | | G (SEQ ID NO: 513) | | | | | | |
| TA16 | MGWS | QVQLVQ | ASTKGPSVFPLAP | GGGGS | QVQLV | GGGGS | DIQMT | DIQMT | RTVAAPSV |
| | CIILFL | SGAEVK | SSKSTSGGTAALG | GGGGS | QSGAE | GGGGS | QSPSSV | QSPSSL | FIFPPSDEQ |
| | VATA | KPGASV | CLVKDYFPEPVTV | GGGGS | VKKPG | GGGGS | SASVG | SASVG | LKSGTASV |
| | TGVH | KVSCKV | SWNSGALTSGVH | (SEQ ID | ASVKV | GGGGS | DRVTIT | DRVTIT | VCLLNNF |
| | S (SEQ | SGYSLS | TFPAVLQSSGLYS | NO: 3) | SCKVSG | (SEQ ID | CRASES | CQASQ | YPREAKV |
| | ID NO: | KYDMS | LSSVVTVPSSSLG | | HTFTGY | NO: 4) | ISSHLA | SPNNL | QWKVDNA |
| | 588) | WVRQA | TQTYICNVNHKPS | | FIHWVR | | WYQQK | LAWY | LQSGNSQE |
| | | PGKGLE | NTKVDKKVEPKS | | QAPGK | | PGKAP | QQKPG | SVTEQDSK |
| | | WMGIIY | CDKTHTCPPCPAP | | GLEWM | | KLLIYA | KAPKL | DSTYSLSS |
| | | TSGYTD | EAAGAPSVFLFPP | | GGMDPI | | GSSRAT | LIYGAS | TLTLSKAD |
| | | YAQKFQ | KPKDTLMISRTPE | | SGATIY | | GVPSRF | DLPSG | YEKHKVY |
| | | GRVTMT | VTCVVVDVSHED | | AQKFQ | | SGSGSG | VPSRFS | ACEVTHQ |
| | | EDTSTD | PEVKFNWYVDGV | | GRVTM | | TDFTLT | GSGSG | GLSSPVTK |
| | | TAYMEL | EVHNAKTKPREE | | TEDTST | | ISSLQPE | TDFTL | SFNRGEC |
| | | SSLRSED | QYNSTYRVVSVL | | DTAYM | | DFANY | TISSLQ | (SEQ ID |
| | | TAVYYC | TVLHQDWLNGKE | | ELSSLK | | YCHQY | PEDFA | NO: 415) |
| | | ATGNPY | YKCKVSNKALPA | | SEDTAV | | DTLPFT | TYYCQ | |
| | | YTNGFN | PIEKTISKAKGQPR | | YYCAR | | FGGGT | NNYYV | |
| | | SWGQGT | EPQVYTLPPSRDE | | EGTTID | | KVEIK | GPVSY | |
| | | LVTVSS | LTKNQVSLTCLV | | AFDIW | | (SEQ ID | AFGGG | |
| | | (SEQ ID | KGFYPSDIAVEWE | | GQGTM | | NO: 33) | TKVEI | |
| | | NO: 514) | SNGQPENNYKTTP | | VTVSS | | | K (SEQ | |
| | | | PVLDSDGSFFLYS | | (SEQ ID | | | ID NO: | |
| | | | KLTVDKSRWQQG | | NO: 69) | | | 515) | |
| | | | NVFSCSVMHEAL | | | | | | |
| | | | HNHYTQKSLSLSP | | | | | | |
| | | | G (SEQ ID NO: 513) | | | | | | |
| TA17 | MGWS | QVQLVQ | ASTKGPSVFPLAP | GGGGS | EVQLV | GGGGS | DIVMT | DIQMT | RTVAAPSV |
| | CIILFL | SGAEVK | SSKSTSGGTAALG | GGGGS | ESGGGL | GGGGS | QSPLSL | QSPSSL | FIFPPSDEQ |
| | VATA | KPGASV | CLVKDYFPEPVTV | GGGGS | VQPGG | GGGGS | PVTPGE | SASVG | LKSGTASV |
| | TGVH | KVSCKV | SWNSGALTSGVH | (SEQ ID | SLRLSC | GGGGS | PASISC | DRVTIT | VCLLNNF |
| | S (SEQ | SGYSLS | TFPAVLQSSGLYS | NO: 3) | AASGL | (SEQ ID | RSSRSL | CQASQ | YPREAKV |
| | ID NO: | KYDMS | LSSVVTVPSSSLG | | MFSTST | NO: 4) | VHGSG | SPNNL | QWKVDNA |
| | 588) | WVRQA | TQTYICNVNHKPS | | MSWVR | | DNYLH | LAWY | LQSGNSQE |
| | | PGKGLE | NTKVDKKVEPKS | | QAPGK | | WYLQK | QQKPG | SVTEQDSK |
| | | WMGIIY | CDKTHTCPPCPAP | | GLELV | | PGQSPQ | KAPKL | DSTYSLSS |
| | | TSGYTD | EAAGAPSVFLFPP | | ASINPS | | LLIYMT | LIYGAS | TLTLSKAD |
| | | YAQKFQ | KPKDTLMISRTPE | | GSVTY | | SNRAPG | DLPSG | YEKHKVY |
| | | GRVTMT | VTCVVVDVSHED | | YPDSV | | VPDRES | VPSRFS | ACEVTHQ |
| | | EDTSTD | PEVKFNWYVDGV | | KGRFTI | | GSGSGT | GSGSG | GLSSPVTK |
| | | TAYMEL | EVHNAKTKPREE | | SRDNA | | DFTLKI | TDFTL | SFNRGEC |
| | | SSLRSED | QYNSTYRVVSVL | | KNSLYL | | SRVEAE | TISSLQ | (SEQ ID |
| | | TAVYYC | TVLHQDWLNGKE | | QMNSL | | DVGVY | PEDFA | NO: 415) |
| | | ATGNPY | YKCKVSNKALPA | | RAEDT | | YCMQS | TYYCQ | |
| | | YTNGFN | PIEKTISKAKGQPR | | AVYYC | | GHWPP | NNYYV | |
| | | SWGQGT | EPQVYTLPPSRDE | | AREDG | | TFGGGT | GPVSY | |
| | | LVTVSS | LTKNQVSLTCLV | | DSRGE | | KVEIK | AFGGG | |
| | | (SEQ ID | KGFYPSDIAVEWE | | AFDIW | | (SEQ ID | TKVEI | |
| | | NO: 514) | SNGQPENNYKTTP | | GQGTM | | NO: 34) | K (SEQ | |
| | | | PVLDSDGSFFLYS | | VTVSS | | | ID NO: | |
| | | | KLTVDKSRWQQG | | (SEQ ID | | | 515) | |
| | | | NVFSCSVMHEAL | | NO: 70) | | | | |
| | | | HNHYTQKSLSLSP | | | | | | |
| | | | G (SEQ ID NO: 513) | | | | | | |
| TA18 | MGWS | QVQLVQ | ASTKGPSVFPLAP | GGGGS | QVQLV | GGGGS | DIQMT | DIQMT | RTVAAPSV |
| | CIILFL | SGAEVK | SSKSTSGGTAALG | GGGGS | QSGAE | GGGGS | QSPSSV | QSPSSL | FIFPPSDEQ |
| | VATA | KPGASV | CLVKDYFPEPVTV | GGGGS | VKKPG | GGGGS | SASVG | SASVG | LKSGTASV |
| | TGVH | KVSCKV | SWNSGALTSGVH | (SEQ ID | ASVKV | GGGGS | DRVTIT | DRVTIT | VCLLNNF |
| | S (SEQ | SGYSLS | TFPAVLQSSGLYS | NO: 3) | SCKVSG | (SEQ ID | CRASQ | CQASQ | YPREAKV |
| | ID NO: | KYDMS | LSSVVTVPSSSLG | | IDLTTS | NO: 4) | NIDTWL | SPNNL | QWKVDNA |
| | 588) | WVRQA | TQTYICNVNHKPS | | AIHWV | | AWYQQ | LAWY | LQSGNSQE |
| | | PGKGLE | NTKVDKKVEPKS | | RQAPG | | KPGKA | QQKPG | SVTEQDSK |
| | | WMGIIY | CDKTHTCPPCPAP | | KGLEW | | PKLLIY | KAPKL | DSTYSLSS |
| | | TSGYTD | EAAGAPSVFLFPP | | MGGINP | | KASTLA | LIYGAS | TLTLSKAD |
| | | YAQKFQ | KPKDTLMISRTPE | | GTGSTI | | SGVPSR | DLPSG | YEKHKVY |
| | | GRVTMT | VTCVVVDVSHED | | YAQKF | | FSGSGS | VPSRFS | ACEVTHQ |
| | | EDTSTD | PEVKFNWYVDGV | | QGRVT | | GTDFTL | GSGSG | GLSSPVTK |
| | | TAYMEL | EVHNAKTKPREE | | MTEDT | | TISSLQP | TDFTL | SFNRGEC |
| | | SSLRSED | QYNSTYRVVSVL | | STDTAY | | EDFAN | TISSLQ | (SEQ ID |
| | | TAVYYC | TVLHQDWLNGKE | | MELSSL | | YYCQQ | PEDFA | NO: 415) |
| | | ATGNPY | YKCKVSNKALPA | | KSEDTA | | YNEVPL | TYYCQ | |
| | | YTNGFN | PIEKTISKAKGQPR | | VYYCA | | TFGGGT | NNYYV | |
| | | SWGQGT | EPQVYTLPPSRDE | | KEVAA | | KVEIK | GPVSY | |
| | | LVTVSS | LTKNQVSLTCLV | | TGAYY | | (SEQ ID | AFGGG | |
| | | (SEQ ID | KGFYPSDIAVEWE | | YWGQG | | NO: 35) | TKVEI | |
| | | NO: 514) | SNGQPENNYKTTP | | TLVTVS | | | K (SEQ | |
| | | | PVLDSDGSFFLYS | | S (SEQ | | | ID NO: | |
| | | | KLTVDKSRWQQG | | ID NO: | | | 515) | |
| | | | NVFSCSVMHEAL | | 71) | | | | |
| | | | HNHYTQKSLSLSP | | | | | | |
| | | | G (SEQ ID NO: 513) | | | | | | |
| TA19 | MGWS | QVQLVQ | ASTKGPSVFPLAP | GGGGS | EVQLV | GGGGS | DIVMT | DIQMT | RTVAAPSV |
| | CIILFL | SGAEVK | SSKSTSGGTAALG | GGGGS | ESGGGL | GGGGS | QSPLSL | QSPSSL | FIFPPSDEQ |
| | VATA | KPGASV | CLVKDYFPEPVTV | GGGGS | VQPGG | GGGGS | PVTPGE | SASVG | LKSGTASV |
| | TGVH | KVSCKV | SWNSGALTSGVH | (SEQ ID | SLRLSC | GGGGS | PASISC | DRVTIT | VCLLNNF |
| | S (SEQ | SGYSLS | TFPAVLQSSGLYS | NO: 3) | AASGFP | (SEQ ID | RSSQSL | CQASQ | YPREAKV |
| | ID NO: | KYDMS | LSSVVTVPSSSLG | | FSDYV | NO: 4) | LHSTGY | SPNNL | QWKVDNA |
| | 588) | WVRQA | TQTYICNVNHKPS | | MSWVR | | NYLHW | LAWY | LQSGNSQE |
| | | PGKGLE | NTKVDKKVEPKS | | QAPGK | | YLQKP | QQKPG | SVTEQDSK |
| | | WMGIIY | CDKTHTCPPCPAP | | GLELV | | GQSPQL | KAPKL | DSTYSLSS |
| | | TSGYTD | EAAGAPSVFLFPP | | ASISPS | | LIYDAT | LIYGAS | TLTLSKAD |
| | | YAQKFQ | KPKDTLMISRTPE | | GSTTYY | | NRASG | DLPSG | YEKHKVY |
| | | GRVTMT | VTCVVVDVSHED | | PDSVK | | VPDRFS | VPSRFS | ACEVTHQ |
| | | EDTSTD | PEVKFNWYVDGV | | GRFTIS | | GSGSGT | GSGSG | GLSSPVTK |
| | | TAYMEL | EVHNAKTKPREE | | RDNAK | | DFTLKI | TDFTL | SFNRGEC |
| | | SSLRSED | QYNSTYRVVSVL | | NSLYLQ | | SRVEAE | TISSLQ | (SEQ ID |
| | | TAVYYC | TVLHQDWLNGKE | | MNSLR | | DVGVY | PEDFA | NO: 415) |
| | | ATGNPY | YKCKVSNKALPA | | AEDTA | | YCQQY | TYYCQ | |
| | | YTNGFN | PIEKTISKAKGQPR | | VYYCV | | AASPPT | NNYYV | |
| | | SWGQGT | EPQVYTLPPSRDE | | RSGEW | | FGGGT | GPVSY | |
| | | LVTVSS | LTKNQVSLTCLV | | TDAFDI | | KVEIK | AFGGG | |
| | | (SEQ ID | KGFYPSDIAVEWE | | WGQGT | | (SEQ ID | TKVEI | |
| | | NO: 514) | SNGQPENNYKTTP | | LVTVSS | | NO: 36) | K (SEQ | |
| | | | PVLDSDGSFFLYS | | (SEQ ID | | | ID NO: | |
| | | | KLTVDKSRWQQG | | NO: 72) | | | 515) | |
| | | | NVFSCSVMHEAL | | | | | | |
| | | | HNHYTQKSLSLSP | | | | | | |
| | | | G (SEQ ID NO: 513) | | | | | | |
| TA20 | MGWS | QVQLVQ | ASTKGPSVFPLAP | GGGGS | QVQLV | GGGGS | DIQMT | DIQMT | RTVAAPSV |
| | CIILFL | SGAEVK | SSKSTSGGTAALG | GGGGS | QSGAE | GGGGS | QSPSSV | QSPSSL | FIFPPSDEQ |
| | VATA | KPGASV | CLVKDYFPEPVTV | GGGGS | VKKPG | GGGGS | SASVG | SASVG | LKSGTASV |
| | TGVH | KVSCKV | SWNSGALTSGVH | (SEQ ID | ASVKV | GGGGS | DRVTIT | DRVTIT | VCLLNNF |
| | S (SEQ | SGYSLS | TFPAVLQSSGLYS | NO: 3) | SCKVSG | (SEQ ID | CRASQ | CQASQ | YPREAKV |
| | ID NO: | KYDMS | LSSVVTVPSSSLG | | YTFSDY | NO: 4) | DISTYL | SPNNL | QWKVDNA |
| | 588) | WVRQA | TQTYICNVNHKPS | | VIHWV | | AWYQQ | LAWY | LQSGNSQE |
| | | PGKGLE | NTKVDKKVEPKS | | RQAPG | | KPGKA | QQKPG | SVTEQDSK |
| | | WMGIIY | CDKTHTCPPCPAP | | KGLEW | | PKLLIY | KAPKL | DSTYSLSS |
| | | TSGYTD | EAAGAPSVFLFPP | | MGGINP | | DTSNR | LIYGAS | TLTLSKAD |
| | | YAQKFQ | KPKDTLMISRTPE | | YSGHTI | | ATGIPS | DLPSG | YEKHKVY |
| | | GRVTMT | VTCVVVDVSHED | | YAQKF | | RFSGSG | VPSRFS | ACEVTHQ |
| | | EDTSTD | PEVKFNWYVDGV | | QGRVT | | SGTDFT | GSGSG | GLSSPVTK |
| | | TAYMEL | EVHNAKTKPREE | | MTEDT | | LTISSL | TDFTL | SFNRGEC |
| | | SSLRSED | QYNSTYRVVSVL | | STDTAY | | QPEDFA | TISSLQ | (SEQ ID |
| | | TAVYYC | TVLHQDWLNGKE | | MELSSL | | NYYCQ | PEDFA | NO: 415) |
| | | ATGNPY | YKCKVSNKALPA | | KSEDTA | | QSAKIP | TYYCQ | |
| | | YTNGFN | PIEKTISKAKGQPR | | VYYCA | | FTFGGG | NNYYV | |
| | | SWGQGT | EPQVYTLPPSRDE | | KELDT | | TKVEIK | GPVSY | |
| | | LVTVSS | LTKNQVSLTCLV | | AGNAF | | (SEQ ID | AFGGG | |
| | | (SEQ ID | KGFYPSDIAVEWE | | DIWGQ | | NO: 37) | TKVEI | |
| | | NO: 514) | SNGQPENNYKTTP | | GTMVT | | | K (SEQ | |
| | | | PVLDSDGSFFLYS | | VSS | | | ID NO: | |
| | | | KLTVDKSRWQQG | | (SEQ ID | | | 515) | |
| | | | NVFSCSVMHEAL | | NO: 73) | | | | |
| | | | HNHYTQKSLSLSP | | | | | | |
| | | | G (SEQ ID NO: 513) | | | | | | |
| TA21 | MGWS | QVQLVQ | ASTKGPSVFPLAP | GGGGS | QVQLQ | GGGGS | EIVMTQ | DIQMT | RTVAAPSV |
| | CIILFL | SGAEVK | SSKSTSGGTAALG | GGGGS | ESGPGL | GGGGS | SPATLS | QSPSSL | FIFPPSDEQ |
| | VATA | KPGASV | CLVKDYFPEPVTV | GGGGS | VKPSQT | GGGGS | LSPGER | SASVG | LKSGTASV |
| | TGVH | KVSCKV | SWNSGALTSGVH | (SEQ ID | LSLTCT | GGGGS | ATLSCR | DRVTIT | VCLLNNF |
| | S (SEQ | SGYSLS | TFPAVLQSSGLYS | NO: 3) | VSGAS | (SEQ ID | ASHSVT | CQASQ | YPREAKV |
| | ID NO: | KYDMS | LSSVVTVPSSSLG | | VRDHY | NO: 4) | SNLAW | SPNNL | QWKVDNA |
| | 588) | WVRQA | TQTYICNVNHKPS | | WSWIR | | YQQKP | LAWY | LQSGNSQE |
| | | PGKGLE | NTKVDKKVEPKS | | QPPGK | | GQAPR | QQKPG | SVTEQDSK |
| | | WMGIIY | CDKTHTCPPCPAP | | GLEWIG | | LLIYGA | KAPKL | DSTYSLSS |
| | | TSGYTD | EAAGAPSVFLFPP | | YAHYS | | SSRVPG | LIYGAS | TLTLSKAD |
| | | YAQKFQ | KPKDTLMISRTPE | | GITDYN | | IPARFS | DLPSG | YEKHKVY |
| | | GRVTMT | VTCVVVDVSHED | | PSLKSR | | GSGSGT | VPSRFS | ACEVTHQ |
| | | EDTSTD | PEVKFNWYVDGV | | VTMSV | | DFTLTI | GSGSG | GLSSPVTK |
| | | TAYMEL | EVHNAKTKPREE | | DTSKN | | SSLEPE | TDFTL | SFNRGEC |
| | | SSLRSED | QYNSTYRVVSVL | | QFSLKV | | DFAVY | TISSLQ | (SEQ ID |
| | | TAVYYC | TVLHQDWLNGKE | | NSVTA | | YCQQY | PEDFA | NO: 415) |
| | | ATGNPY | YKCKVSNKALPA | | ADTAV | | DNRPIT | TYYCQ | |
| | | YTNGFN | PIEKTISKAKGQPR | | YYCAIY | | FGGGT | NNYYV | |
| | | SWGQGT | EPQVYTLPPSRDE | | SSGWW | | KVEIK | GPVSY | |
| | | LVTVSS | LTKNQVSLTCLV | | EDYWG | | (SEQ ID | AFGGG | |
| | | (SEQ ID | KGFYPSDIAVEWE | | QGTLV | | NO: 38) | TKVEI | |
| | | NO: 514) | SNGQPENNYKTTP | | TVSS | | | K (SEQ | |
| | | | PVLDSDGSFFLYS | | (SEQ ID | | | ID NO: | |
| | | | KLTVDKSRWQQG | | NO: 74) | | | 515) | |
| | | | NVFSCSVMHEAL | | | | | | |
| | | | HNHYTQKSLSLSP | | | | | | |
| | | | G (SEQ ID NO: 513) | | | | | | |
| TA22 | MGWS | QVQLVQ | ASTKGPSVFPLAP | GGGGS | QVQLV | GGGGS | DIQMT | DIQMT | RTVAAPSV |
| | CIILFL | SGAEVK | SSKSTSGGTAALG | GGGGS | QSGAE | GGGGS | QSPSSV | QSPSSL | FIFPPSDEQ |
| | VATA | KPGASV | CLVKDYFPEPVTV | GGGGS | VKKPG | GGGGS | SASVG | SASVG | LKSGTASV |
| | TGVH | KVSCKV | SWNSGALTSGVH | (SEQ ID | ASVKV | GGGGS | DRVTIT | DRVTIT | VCLLNNF |
| | S (SEQ | SGYSLS | TFPAVLQSSGLYS | NO: 3) | SCKVSG | (SEQ ID | CRASQS | CQASQ | YPREAKV |
| | ID NO: | KYDMS | LSSVVTVPSSSLG | | YPFPSY | NO: 4) | IAPLAW | SPNNL | QWKVDNA |
| | 588) | WVRQA | TQTYICNVNHKPS | | DIHWV | | YQQKP | LAWY | LQSGNSQE |
| | | PGKGLE | NTKVDKKVEPKS | | RQAPG | | GKAPK | QQKPG | SVTEQDSK |
| | | WMGIIY | CDKTHTCPPCPAP | | KGLEW | | LLIYAA | KAPKL | DSTYSLSS |
| | | TSGYTD | EAAGAPSVFLFPP | | MGGMN | | STLQPG | LIYGAS | TLTLSKAD |
| | | YAQKFQ | KPKDTLMISRTPE | | PTTGDT | | VPSRFS | DLPSG | YEKHKVY |
| | | GRVTMT | VTCVVVDVSHED | | IYAQKF | | GSGSGT | VPSRFS | ACEVTHQ |
| | | EDTSTD | PEVKFNWYVDGV | | QGRVT | | DFTLTI | GSGSG | GLSSPVTK |
| | | TAYMEL | EVHNAKTKPREE | | MTEDT | | SSLQPE | TDFTL | SFNRGEC |
| | | SSLRSED | QYNSTYRVVSVL | | STDTAY | | DFANY | TISSLQ | (SEQ ID |
| | | TAVYYC | TVLHQDWLNGKE | | MELSSL | | YCLQY | PEDFA | NO: 415) |
| | | ATGNPY | YKCKVSNKALPA | | KSEDTA | | HVLPIT | TYYCQ | |
| | | YTNGFN | PIEKTISKAKGQPR | | VYYCA | | FGGGT | NNYYV | |
| | | SWGQGT | EPQVYTLPPSRDE | | RETGG | | KVEIK | GPVSY | |
| | | LVTVSS | LTKNQVSLTCLV | | GEMAF | | (SEQ ID | AFGGG | |
| | | (SEQ ID | KGFYPSDIAVEWE | | DIWGQ | | NO: 39) | TKVEI | |
| | | NO: 514) | SNGQPENNYKTTP | | GTMVT | | | K (SEQ | |
| | | | PVLDSDGSFFLYS | | VSS | | | ID NO: | |
| | | | KLTVDKSRWQQG | | (SEQ ID | | | 515) | |
| | | | NVFSCSVMHEAL | | NO: 75) | | | | |
| | | | HNHYTQKSLSLSP | | | | | | |
| | | | G (SEQ ID NO: 513) | | | | | | |
| TA23 | MGWS | QVQLVQ | ASTKGPSVFPLAP | GGGGS | QVQLQ | GGGGS | EIVMTQ | DIQMT | RTVAAPSV |
| | CIILFL | SGAEVK | SSKSTSGGTAALG | GGGGS | ESGPGL | GGGGS | SPATLS | QSPSSL | FIFPPSDEQ |
| | VATA | KPGASV | CLVKDYFPEPVTV | GGGGS | VKPSQT | GGGGS | LSPGER | SASVG | LKSGTASV |
| | TGVH | KVSCKV | SWNSGALTSGVH | (SEQ ID | LSLTCT | GGGGS | ATLSCR | DRVTIT | VCLLNNF |
| | S (SEQ | SGYSLS | TFPAVLQSSGLYS | NO: 3) | VSEYAI | (SEQ ID | ASQNIG | CQASQ | YPREAKV |
| | ID NO: | KYDMS | LSSVVTVPSSSLG | | RSDYT | NO: 4) | TNLAW | SPNNL | QWKVDNA |
| | 588) | WVRQA | TQTYICNVNHKPS | | WSWIR | | YQQKP | LAWY | LQSGNSQE |
| | | PGKGLE | NTKVDKKVEPKS | | QPPGK | | GQAPR | QQKPG | SVTEQDSK |
| | | WMGIIY | CDKTHTCPPCPAP | | GLEWIG | | LLIYDA | KAPKL | DSTYSLSS |
| | | TSGYTD | EAAGAPSVFLFPP | | YIHHSG | | SKRPTG | LIYGAS | TLTLSKAD |
| | | YAQKFQ | KPKDTLMISRTPE | | LTDYNP | | IPARFS | DLPSG | YEKHKVY |
| | | GRVTMT | VTCVVVDVSHED | | SLKSRV | | GSGSGT | VPSRFS | ACEVTHQ |
| | | EDTSTD | PEVKFNWYVDGV | | TMSVD | | DFTLTI | GSGSG | GLSSPVTK |
| | | TAYMEL | EVHNAKTKPREE | | TSKNQF | | SSLEPE | TDFTL | SFNRGEC |
| | | SSLRSED | QYNSTYRVVSVL | | SLKVNS | | DFAVY | TISSLQ | (SEQ ID |
| | | TAVYYC | TVLHQDWLNGKE | | VTAAD | | YCQQY | PEDFA | NO: 415) |
| | | ATGNPY | YKCKVSNKALPA | | TAVYY | | GTTPFT | TYYCQ | |
| | | YTNGFN | PIEKTISKAKGQPR | | CARVR | | FGGGT | NNYYV | |
| | | SWGQGT | EPQVYTLPPSRDE | | YSSSSE | | KVEIK | GPVSY | |
| | | LVTVSS | LTKNQVSLTCLV | | GWFDP | | (SEQ ID | AFGGG | |
| | | (SEQ ID | KGFYPSDIAVEWE | | WGQGT | | NO: 40) | TKVEI | |
| | | NO: 514) | SNGQPENNYKTTP | | LVTVSS | | | K (SEQ | |
| | | | PVLDSDGSFFLYS | | (SEQ ID | | | ID NO: | |
| | | | KLTVDKSRWQQG | | NO: 76) | | | 515) | |
| | | | NVFSCSVMHEAL | | | | | | |
| | | | HNHYTQKSLSLSP | | | | | | |
| | | | G (SEQ ID NO: 513) | | | | | | |
| TA24 | MGWS | QVQLVQ | ASTKGPSVFPLAP | GGGGS | QVQLQ | GGGGS | EIVMTQ | DIQMT | RTVAAPSV |
| | CIILFL | SGAEVK | SSKSTSGGTAALG | GGGGS | ESGPGL | GGGGS | SPATLS | QSPSSL | FIFPPSDEQ |
| | VATA | KPGASV | CLVKDYFPEPVTV | GGGGS | VKPSQT | GGGGS | LSPGER | SASVG | LKSGTASV |
| | TGVH | KVSCKV | SWNSGALTSGVH | (SEQ ID | LSLTCT | GGGGS | ATLSCR | DRVTIT | VCLLNNF |
| | S (SEQ | SGYSLS | TFPAVLQSSGLYS | NO: 3) | VSGASI | (SEQ ID | ASESIG | CQASQ | YPREAKV |
| | ID NO: | KYDMS | LSSVVTVPSSSLG | | STSDT | NO: 4) | SNLAW | SPNNL | QWKVDNA |
| | 588) | WVRQA | TQTYICNVNHKPS | | WSWIR | | YQQKP | LAWY | LQSGNSQE |
| | | PGKGLE | NTKVDKKVEPKS | | QPPGK | | GQAPR | QQKPG | SVTEQDSK |
| | | WMGIIY | CDKTHTCPPCPAP | | GLEWIG | | LLIYDA | KAPKL | DSTYSLSS |
| | | TSGYTD | EAAGAPSVFLFPP | | YIHHSG | | SNRAT | LIYGAS | TLTLSKAD |
| | | YAQKFQ | KPKDTLMISRTPE | | ITDYNP | | GIPARF | DLPSG | YEKHKVY |
| | | GRVTMT | VTCVVVDVSHED | | SLKSRV | | SGSGSG | VPSRFS | ACEVTHQ |
| | | EDTSTD | PEVKFNWYVDGV | | TMSVD | | TDFTLT | GSGSG | GLSSPVTK |
| | | TAYMEL | EVHNAKTKPREE | | TSKNQF | | ISSLEPE | TDFTL | SFNRGEC |
| | | SSLRSED | QYNSTYRVVSVL | | SLKVNS | | DFAVY | TISSLQ | (SEQ ID |
| | | TAVYYC | TVLHQDWLNGKE | | VTAAD | | YCQQY | PEDFA | NO: 415) |
| | | ATGNPY | YKCKVSNKALPA | | TAVYY | | DHWPL | TYYCQ | |
| | | YTNGFN | PIEKTISKAKGQPR | | CARGG | | TFGGGT | NNYYV | |
| | | SWGQGT | EPQVYTLPPSRDE | | SSGNW | | KVEIK | GPVSY | |
| | | LVTVSS | LTKNQVSLTCLV | | YLLDY | | (SEQ ID | AFGGG | |
| | | (SEQ ID | KGFYPSDIAVEWE | | WGQGT | | NO: 41) | TKVEI | |
| | | NO: 514) | SNGQPENNYKTTP | | LVTVSS | | | K (SEQ | |
| | | | PVLDSDGSFFLYS | | (SEQ ID | | | ID NO: | |
| | | | KLTVDKSRWQQG | | NO: 77) | | | 515) | |
| | | | NVFSCSVMHEAL | | | | | | |
| | | | HNHYTQKSLSLSP | | | | | | |
| | | | G (SEQ ID NO: 513) | | | | | | |
| TA25 | MGWS | QVQLVQ | ASTKGPSVFPLAP | GGGGS | EVQLV | GGGGS | DIVMT | DIQMT | RTVAAPSV |
| | CIILFL | SGAEVK | SSKSTSGGTAALG | GGGGS | ESGGGL | GGGGS | QSPLSL | QSPSSL | FIFPPSDEQ |
| | VATA | KPGASV | CLVKDYFPEPVTV | GGGGS | VQPGG | GGGGS | PVTPGE | SASVG | LKSGTASV |
| | TGVH | KVSCKV | SWNSGALTSGVH | (SEQ ID | SLRLSC | GGGGS | PASISC | DRVTIT | VCLLNNF |
| | S (SEQ | SGYSLS | TFPAVLQSSGLYS | NO: 3) | AASRFT | (SEQ ID | RSSRSL | CQASQ | YPREAKV |
| | ID NO: | KYDMS | LSSVVTVPSSSLG | | FSDYH | NO: 4) | VHGSG | SPNNL | QWKVDNA |
| | 588) | WVRQA | TQTYICNVNHKPS | | MSWVR | | DNYLH | LAWY | LQSGNSQE |
| | | PGKGLE | NTKVDKKVEPKS | | QAPGK | | WYLQK | QQKPG | SVTEQDSK |
| | | WMGIIY | CDKTHTCPPCPAP | | GLELV | | PGQSPQ | KAPKL | DSTYSLSS |
| | | TSGYTD | EAAGAPSVFLFPP | | ASIDTE | | LLIYMA | LIYGAS | TLTLSKAD |
| | | YAQKFQ | KPKDTLMISRTPE | | GKTYY | | SNRAPG | DLPSG | YEKHKVY |
| | | GRVTMT | VTCVVVDVSHED | | PDSVK | | VPDRFS | VPSRFS | ACEVTHQ |
| | | EDTSTD | PEVKFNWYVDGV | | GRFTIS | | GSGSGT | GSGSG | GLSSPVTK |
| | | TAYMEL | EVHNAKTKPREE | | RDNAK | | DFTLKI | TDFTL | SFNRGEC |
| | | SSLRSED | QYNSTYRVVSVL | | NSLYLQ | | SRVEAE | TISSLQ | (SEQ ID |
| | | TAVYYC | TVLHQDWLNGKE | | MNSLR | | DVGVY | PEDFA | NO: 415) |
| | | ATGNPY | YKCKVSNKALPA | | AEDTA | | YCMQA | TYYCQ | |
| | | YTNGFN | PIEKTISKAKGQPR | | VYYCA | | LRAPFS | NNYYV | |
| | | SWGQGT | EPQVYTLPPSRDE | | RDVGD | | FGGGT | GPVSY | |
| | | LVTVSS | LTKNQVSLTCLV | | WYFDL | | KVEIK | AFGGG | |
| | | (SEQ ID | KGFYPSDIAVEWE | | WGRGT | | (SEQ ID | TKVEI | |
| | | NO: 514) | SNGQPENNYKTTP | | LVTVSS | | NO: 42) | K (SEQ | |
| | | | PVLDSDGSFFLYS | | (SEQ ID | | | ID NO: | |
| | | | KLTVDKSRWQQG | | NO: 78) | | | 515) | |
| | | | NVFSCSVMHEAL | | | | | | |
| | | | HNHYTQKSLSLSP | | | | | | |
| | | | G (SEQ ID NO: 513) | | | | | | |
| TA26 | MGWS | QVQLVQ | ASTKGPSVFPLAP | GGGGS | QVQLQ | GGGGS | EIVMTQ | DIQMT | RTVAAPSV |
| | CIILFL | SGAEVK | SSKSTSGGTAALG | GGGGS | ESGPGL | GGGGS | SPATLS | QSPSSL | FIFPPSDEQ |
| | VATA | KPGASV | CLVKDYFPEPVTV | GGGGS | VKPSQT | GGGGS | LSPGER | SASVG | LKSGTASV |
| | TGVH | KVSCKV | SWNSGALTSGVH | (SEQ ID | LSLTCT | GGGGS | ATLSCR | DRVTIT | VCLLNNF |
| | S (SEQ | SGYSLS | TFPAVLQSSGLYS | NO: 3) | VSGVSL | (SEQ ID | ASQSLS | CQASQ | YPREAKV |
| | ID NO: | KYDMS | LSSVVTVPSSSLG | | SNARM | NO: 4) | SSHLA | SPNNL | QWKVDNA |
| | 588) | WVRQA | TQTYICNVNHKPS | | GWSWI | | WYQQK | LAWY | LQSGNSQE |
| | | PGKGLE | NTKVDKKVEPKS | | RQPPGK | | PGQAPR | QQKPG | SVTEQDSK |
| | | WMGIIY | CDKTHTCPPCPAP | | GLEWIG | | LLIYDA | KAPKL | DSTYSLSS |
| | | TSGYTD | EAAGAPSVFLFPP | | YVHTS | | SIRVPGI | LIYGAS | TLTLSKAD |
| | | YAQKFQ | KPKDTLMISRTPE | | GSTDY | | PARFSG | DLPSG | YEKHKVY |
| | | GRVTMT | VTCVVVDVSHED | | NPSLKS | | SGSGTD | VPSRFS | ACEVTHQ |
| | | EDTSTD | PEVKFNWYVDGV | | RVTMS | | FTLTISS | GSGSG | GLSSPVTK |
| | | TAYMEL | EVHNAKTKPREE | | VDTSK | | LEPEDF | TDFTL | SFNRGEC |
| | | SSLRSED | QYNSTYRVVSVL | | NQFSLK | | AVYYC | TISSLQ | (SEQ ID |
| | | TAVYYC | TVLHQDWLNGKE | | VNSVT | | QQYAV | PEDFA | NO: 415) |
| | | ATGNPY | YKCKVSNKALPA | | AADTA | | PPITFG | TYYCQ | |
| | | YTNGFN | PIEKTISKAKGQPR | | VYYCA | | GGTKV | NNYYV | |
| | | SWGQGT | EPQVYTLPPSRDE | | RDAGN | | EIK | GPVSY | |
| | | LVTVSS | LTKNQVSLTCLV | | WFDPW | | (SEQ ID | AFGGG | |
| | | (SEQ ID | KGFYPSDIAVEWE | | GQGTL | | NO: 43) | TKVEI | |
| | | NO: 514) | SNGQPENNYKTTP | | VTVSS | | | K (SEQ | |
| | | | PVLDSDGSFFLYS | | (SEQ ID | | | ID NO: | |
| | | | KLTVDKSRWQQG | | NO: 79) | | | 515) | |
| | | | NVFSCSVMHEAL | | | | | | |
| | | | HNHYTQKSLSLSP | | | | | | |
| | | | G (SEQ ID NO: 513) | | | | | | |
| TA27 | MGWS | QVQLVQ | ASTKGPSVFPLAP | GGGGS | EVQLV | GGGGS | DIQMT | DIQMT | RTVAAPSV |
| | CIILFL | SGAEVK | SSKSTSGGTAALG | GGGGS | ESGGGL | GGGGS | QSPSSL | QSPSSL | FIFPPSDEQ |
| | VATA | KPGASV | CLVKDYFPEPVTV | GGGGS | VQPGRS | GGGGS | SASVG | SASVG | LKSGTASV |
| | TGVH | KVSCKV | SWNSGALTSGVH | (SEQ ID | LRLSCA | GGGGS | DRVTIT | DRVTIT | VCLLNNF |
| | S (SEQ | SGYSLS | TFPAVLQSSGLYS | NO: 3) | ASGSTL | (SEQ ID | CRASQ | CQASQ | YPREAKV |
| | ID NO: | KYDMS | LSSVVTVPSSSLG | | SSYGM | NO: 4) | GIGSYL | SPNNL | QWKVDNA |
| | 588) | WVRQA | TQTYICNVNHKPS | | HWVRQ | | AWYQQ | LAWY | LQSGNSQE |
| | | PGKGLE | NTKVDKKVEPKS | | APGKG | | KPGKA | QQKPG | SVTEQDSK |
| | | WMGIIY | CDKTHTCPPCPAP | | LEWVS | | PKLLIY | KAPKL | DSTYSLSS |
| | | TSGYTD | EAAGAPSVFLFPP | | AISYDA | | EASRLE | LIYGAS | TLTLSKAD |
| | | YAQKFQ | KPKDTLMISRTPE | | STIDYA | | SGVPSR | DLPSG | YEKHKVY |
| | | GRVTMT | VTCVVVDVSHED | | DSVEG | | FSGSGS | VPSRFS | ACEVTHQ |
| | | EDTSTD | PEVKFNWYVDGV | | RFTISR | | GTDFTL | GSGSG | GLSSPVTK |
| | | TAYMEL | EVHNAKTKPREE | | DNAKN | | TISSLQP | TDFTL | SFNRGEC |
| | | SSLRSED | QYNSTYRVVSVL | | SLYLQ | | EDVAT | TISSLQ | (SEQ ID |
| | | TAVYYC | TVLHQDWLNGKE | | MNSLR | | YYCQQ | PEDFA | NO: 415) |
| | | ATGNPY | YKCKVSNKALPA | | AEDTA | | GYNAPI | TYYCQ | |
| | | YTNGFN | PIEKTISKAKGQPR | | VYYCA | | TFGGGT | NNYYV | |
| | | SWGQGT | EPQVYTLPPSRDE | | RDLLG | | KVEIK | GPVSY | |
| | | LVTVSS | LTKNQVSLTCLV | | YGMDV | | (SEQ ID | AFGGG | |
| | | (SEQ ID | KGFYPSDIAVEWE | | WGQGT | | NO: 44) | TKVEI | |
| | | NO: 514) | SNGQPENNYKTTP | | MVTVS | | | K (SEQ | |
| | | | PVLDSDGSFFLYS | | S (SEQ | | | ID NO: | |
| | | | KLTVDKSRWQQG | | ID NO: | | | 515) | |
| | | | NVFSCSVMHEAL | | 80) | | | | |
| | | | HNHYTQKSLSLSP | | | | | | |
| | | | G (SEQ ID NO: 513) | | | | | | |
| TA28 | MGWS | QVQLVQ | ASTKGPSVFPLAP | GGGGS | QVQLV | GGGGS | DIQMT | DIQMT | RTVAAPSV |
| | CIILFL | SGAEVK | SSKSTSGGTAALG | GGGGS | QSGAE | GGGGS | QSPSSV | QSPSSL | FIFPPSDEQ |
| | VATA | KPGASV | CLVKDYFPEPVTV | GGGGS | VKKPG | GGGGS | SASVG | SASVG | LKSGTASV |
| | TGVH | KVSCKV | SWNSGALTSGVH | (SEQ II | ASVKV | GGGGS | DRVTIT | DRVTIT | VCLLNNF |
| | S (SEQ | SGYSLS | TFPAVLQSSGLYS | NO: 3) | SCKVSG | (SEQ ID | CRASQ | CQASQ | YPREAKV |
| | ID NO: | KYDMS | LSSVVTVPSSSLG | | SAFTSN | NO: 4) | DIGNW | SPNNL | QWKVDNA |
| | 588) | WVRQA | TQTYICNVNHKPS | | DIHWV | | LAWYQ | LAWY | LQSGNSQE |
| | | PGKGLE | NTKVDKKVEPKS | | RQAPG | | QKPGK | QQKPG | SVTEQDSK |
| | | WMGIIY | CDKTHTCPPCPAP | | KGLEW | | APKLLI | KAPKL | DSTYSLSS |
| | | TSGYTD | EAAGAPSVFLFPP | | MGGINP | | YDASTL | LIYGAS | TLTLSKAD |
| | | YAQKFQ | KPKDTLMISRTPE | | RSGATI | | DTGVPS | DLPSG | YEKHKVY |
| | | GRVTMT | VTCVVVDVSHED | | YAQKF | | RFSGSG | VPSRFS | ACEVTHQ |
| | | EDTSTD | PEVKFNWYVDGV | | QGRVT | | SGTDFT | GSGSG | GLSSPVTK |
| | | TAYMEL | EVHNAKTKPREE | | MTEDT | | LTISSL | TDFTL | SFNRGEC |
| | | SSLRSED | QYNSTYRVVSVL | | STDTAY | | QPEDFA | TISSLQ | (SEQ ID |
| | | TAVYYC | TVLHQDWLNGKE | | MELSSL | | NYYCL | PEDFA | NO: 415) |
| | | ATGNPY | YKCKVSNKALPA | | KSEDTA | | QDYSY | TYYCQ | |
| | | YTNGFN | PIEKTISKAKGQPR | | VYYCA | | PLTFGG | NNYYV | |
| | | SWGQGT | EPQVYTLPPSRDE | | RALSD | | GTKVEI | GPVSY | |
| | | LVTVSS | LTKNQVSLTCLV | | DSSGY | | K (SEQ | AFGGG | |
| | | (SEQ ID | KGFYPSDIAVEWE | | DAFDI | | ID NO: | TKVEI | |
| | | NO: 514) | SNGQPENNYKTTP | | WGQGT | | 45) | K (SEQ | |
| | | | PVLDSDGSFFLYS | | MVTVS | | | ID NO: | |
| | | | KLTVDKSRWQQG | | S (SEQ | | | 515) | |
| | | | NVFSCSVMHEAL | | ID NO:81) | | | | |
| | | | HNHYTQKSLSLSP | | | | | | |
| | | | G (SEQ ID NO: 513) | | | | | | |
| TA29 | MGWS | QVQLVQ | ASTKGPSVFPLAP | GGGGS | EVQLV | GGGGS | DIQMT | DIQMT | RTVAAPSV |
| | CIILFL | SGAEVK | SSKSTSGGTAALG | GGGGS | ESGGGL | GGGGS | QSPSSL | QSPSSL | FIFPPSDEQ |
| | VATA | KPGASV | CLVKDYFPEPVTV | GGGGS | VQPGRS | GGGGS | SASVG | SASVG | LKSGTASV |
| | TGVH | KVSCKV | SWNSGALTSGVH | (SEQ ID | LRLSCA | GGGGS | DRVTIT | DRVTIT | VCLLNNF |
| | S (SEQ | SGYSLS | TFPAVLQSSGLYS | NO: 3) | ASGSTL | (SEQ ID | CRASQ | CQASQ | YPREAKV |
| | ID NO: | KYDMS | LSSVVTVPSSSLG | | SSYGM | NO: 4) | DISNWL | SPNNL | QWKVDNA |
| | 588) | WVRQA | TQTYICNVNHKPS | | HWVRQ | | AWYQQ | LAWY | LQSGNSQE |
| | | PGKGLE | NTKVDKKVEPKS | | APGKG | | KPGKA | QQKPG | SVTEQDSK |
| | | WMGIIY | CDKTHTCPPCPAP | | LEWVS | | PKLLIY | KAPKL | DSTYSLSS |
| | | TSGYTD | EAAGAPSVFLFPP | | AISYDA | | EASRLE | LIYGAS | TLTLSKAD |
| | | YAQKFQ | KPKDTLMISRTPE | | STIDYA | | SGVPSR | DLPSG | YEKHKVY |
| | | GRVTMT | VTCVVVDVSHED | | DSVEG | | FSGSGS | VPSRFS | ACEVTHQ |
| | | EDTSTD | PEVKFNWYVDGV | | RFTISR | | GTDFTL | GSGSG | GLSSPVTK |
| | | TAYMEL | EVHNAKTKPREE | | DNAKN | | TISSLQP | TDFTL | SFNRGEC |
| | | SSLRSED | QYNSTYRVVSVL | | SLYLQ | | EDVAT | TISSLQ | (SEQ ID |
| | | TAVYYC | TVLHQDWLNGKE | | MNSLR | | YYCQQ | PEDFA | NO: 415) |
| | | ATGNPY | YKCKVSNKALPA | | AEDTA | | GYNAPI | TYYCQ | |
| | | YTNGFN | PIEKTISKAKGQPR | | VYYCA | | TFGGGT | NNYYV | |
| | | SWGQGT | EPQVYTLPPSRDE | | RDLLG | | KVEIK | GPVSY | |
| | | LVTVSS | LTKNQVSLTCLV | | YGMDV | | (SEQ ID | AFGGG | |
| | | (SEQ ID | KGFYPSDIAVEWE | | WGQGT | | NO: 46) | TKVEI | |
| | | NO: 514) | SNGQPENNYKTTP | | MVTVS | | | K (SEQ | |
| | | | PVLDSDGSFFLYS | | S (SEQ | | | ID NO: | |
| | | | KLTVDKSRWQQG | | ID NO: | | | 515) | |
| | | | NVFSCSVMHEAL | | 80) | | | | |
| | | | HNHYTQKSLSLSP | | | | | | |
| | | | G (SEQ ID NO: 513) | | | | | | |
| TA30 | MGWS | QVQLVQ | ASTKGPSVFPLAP | GGGGS | EVQLV | GGGGS | DIQMT | DIQMT | RTVAAPSV |
| | CIILFL | SGAEVK | SSKSTSGGTAALG | GGGGS | ESGGGL | GGGGS | QSPSSL | QSPSSL | FIFPPSDEQ |
| | VATA | KPGASV | CLVKDYFPEPVTV | GGGGS | VQPGRS | GGGGS | SASVG | SASVG | LKSGTASV |
| | TGVH | KVSCKV | SWNSGALTSGVH | (SEQ ID | LRLSCA | GGGGS | DRVTIT | DRVTIT | VCLLNNF |
| | S (SEQ | SGYSLS | TFPAVLQSSGLYS | NO: 3) | ASGFNF | (SEQ ID | CRASQ | CQASQ | YPREAKV |
| | ID NO: | KYDMS | LSSVVTVPSSSLG | | GAFAM | NO: 4) | DISNWL | SPNNL | QWKVDNA |
| | 588) | WVRQA | TQTYICNVNHKPS | | HWVRQ | | AWYQQ | LAWY | LQSGNSQE |
| | | PGKGLE | NTKVDKKVEPKS | | APGKG | | KPGKA | QQKPG | SVTEQDSK |
| | | WMGIIY | CDKTHTCPPCPAP | | LEWVS | | PKLLIY | KAPKL | DSTYSLSS |
| | | TSGYTD | EAAGAPSVFLFPP | | AINQGG | | DASSLV | LIYGAS | TLTLSKAD |
| | | YAQKFQ | KPKDTLMISRTPE | | SEVDY | | SGVPSR | DLPSG | YEKHKVY |
| | | GRVTMT | VTCVVVDVSHED | | ADSVE | | FSGSGS | VPSRFS | ACEVTHQ |
| | | EDTSTD | PEVKFNWYVDGV | | GRFTIS | | GTDFTL | GSGSG | GLSSPVTK |
| | | TAYMEL | EVHNAKTKPREE | | RDNAK | | TISSLQP | TDFTL | SFNRGEC |
| | | SSLRSED | QYNSTYRVVSVL | | NSLYLQ | | EDVAT | TISSLQ | (SEQ ID |
| | | TAVYYC | TVLHQDWLNGKE | | MNSLR | | YYCHQI | PEDFA | NO: 415) |
| | | ATGNPY | YKCKVSNKALPA | | AEDTA | | YDTPPT | TYYCQ | |
| | | YTNGFN | PIEKTISKAKGQPR | | VYYCA | | FGGGT | NNYYV | |
| | | SWGQGT | EPQVYTLPPSRDE | | RDPGLP | | KVEIK | GPVSY | |
| | | LVTVSS | LTKNQVSLTCLV | | VSAFDI | | (SEQ ID | AFGGG | |
| | | (SEQ ID | KGFYPSDIAVEWE | | WGLGT | | NO: 47) | TKVEI | |
| | | NO: 514) | SNGQPENNYKTTP | | MVTVS | | | K (SEQ | |
| | | | PVLDSDGSFFLYS | | S (SEQ | | | ID NO: | |
| | | | KLTVDKSRWQQG | | ID NO: | | | 515) | |
| | | | NVFSCSVMHEAL | | 82) | | | | |
| | | | HNHYTQKSLSLSP | | | | | | |
| | | | G (SEQ ID NO: 513) | | | | | | |
| TA31 | MGWS | QVQLVQ | ASTKGPSVFPLAP | GGGGS | EVQLV | GGGGS | DIQMT | DIQMT | RTVAAPSV |
| | CIILFL | SGAEVK | SSKSTSGGTAALG | GGGGS | ESGGGL | GGGGS | QSPSSL | QSPSSL | FIFPPSDEQ |
| | VATA | KPGASV | CLVKDYFPEPVTV | GGGGS | VQPGRS | GGGGS | SASVG | SASVG | LKSGTASV |
| | TGVH | KVSCKV | SWNSGALTSGVH | (SEQ ID | LRLSCA | GGGGS | DRVTIT | DRVTIT | VCLLNNF |
| | S (SEQ | SGYSLS | TFPAVLQSSGLYS | NO: 3) | ASGFTF | (SEQ ID | CRASQ | CQASQ | YPREAKV |
| | ID NO: | KYDMS | LSSVVTVPSSSLG | | ENYGM | NO: 4) | AISGSL | SPNNL | QWKVDNA |
| | 588) | WVRQA | TQTYICNVNHKPS | | HWVRQ | | AWYQQ | LAWY | LQSGNSQE |
| | | PGKGLE | NTKVDKKVEPKS | | APGKG | | KPGKA | QQKPG | SVTEQDSK |
| | | WMGIIY | CDKTHTCPPCPAP | | LEWVS | | PKLLIY | KAPKL | DSTYSLSS |
| | | TSGYTD | EAAGAPSVFLFPP | | AISYDA | | ATSRLE | LIYGAS | TLTLSKAD |
| | | YAQKFQ | KPKDTLMISRTPE | | STIDYA | | SGVPSR | DLPSG | YEKHKVY |
| | | GRVTMT | VTCVVVDVSHED | | DSVEG | | FSGSGS | VPSRFS | ACEVTHQ |
| | | EDTSTD | PEVKFNWYVDGV | | RFTISR | | GTDFTL | GSGSG | GLSSPVTK |
| | | TAYMEL | EVHNAKTKPREE | | DNAKN | | TISSLQP | TDFTL | SFNRGEC |
| | | SSLRSED | QYNSTYRVVSVL | | SLYLQ | | EDVAT | TISSLQ | (SEQ ID |
| | | TAVYYC | TVLHQDWLNGKE | | MNSLR | | YYCQQ | PEDFA | NO: 415) |
| | | ATGNPY | YKCKVSNKALPA | | AEDTA | | SGSTPIT | TYYCQ | |
| | | YTNGFN | PIEKTISKAKGQPR | | VYYCA | | FGGGT | NNYYV | |
| | | SWGQGT | EPQVYTLPPSRDE | | SEYGLA | | KVEIK | GPVSY | |
| | | LVTVSS | LTKNQVSLTCLV | | FDQWG | | (SEQ ID | AFGGG | |
| | | (SEQ ID | KGFYPSDIAVEWE | | QGTLV | | NO: 48) | TKVEI | |
| | | NO: 514) | SNGQPENNYKTTP | | TVSS | | | K (SEQ | |
| | | | PVLDSDGSFFLYS | | (SEQ ID | | | ID NO: | |
| | | | KLTVDKSRWQQG | | NO: 83) | | | 515) | |
| | | | NVFSCSVMHEAL | | | | | | |
| | | | HNHYTQKSLSLSP | | | | | | |
| | | | G (SEQ ID NO: 513) | | | | | | |
| TA32 | MGWS | QVQLVQ | ASTKGPSVFPLAP | GGGGS | EVQLV | GGGGS | DIQMT | DIQMT | RTVAAPSV |
| | CIILFL | SGAEVK | SSKSTSGGTAALG | GGGGS | ESGGGL | GGGGS | QSPSSL | QSPSSL | FIFPPSDEQ |
| | VATA | KPGASV | CLVKDYFPEPVTV | GGGGS | VQPGRS | GGGGS | SASVG | SASVG | LKSGTASV |
| | TGVH | KVSCKV | SWNSGALTSGVH | (SEQ ID | LRLSCA | GGGGS | DRVTIT | DRVTIT | VCLLNNF |
| | S (SEQ | SGYSLS | TFPAVLQSSGLYS | NO: 3) | ASGSTL | (SEQ ID | CRASQ | CQASQ | YPREAKV |
| | ID NO: | KYDMS | LSSVVTVPSSSLG | | SSYGM | NO: 4) | GIGSYL | SPNNL | QWKVDNA |
| | 588) | WVRQA | TQTYICNVNHKPS | | HWVRQ | | AWYQQ | LAWY | LQSGNSQE |
| | | PGKGLE | NTKVDKKVEPKS | | APGKG | | KPGKA | QQKPG | SVTEQDSK |
| | | WMGIIY | CDKTHTCPPCPAP | | LEWVS | | PKLLIY | KAPKL | DSTYSLSS |
| | | TSGYTD | EAAGAPSVFLFPP | | AISYDA | | EASRLE | LIYGAS | TLTLSKAD |
| | | YAQKFQ | KPKDTLMISRTPE | | STIDYA | | SGVPSR | DLPSG | YEKHKVY |
| | | GRVTMT | VTCVVVDVSHED | | DSVEG | | FSGSGS | VPSRFS | ACEVTHQ |
| | | EDTSTD | PEVKFNWYVDGV | | RFTISR | | GTDFTL | GSGSG | GLSSPVTK |
| | | TAYMEL | EVHNAKTKPREE | | DNAKN | | TISSLQP | TDFTL | SFNRGEC |
| | | SSLRSED | QYNSTYRVVSVL | | SLYLQ | | EDVAT | TISSLQ | (SEQ ID |
| | | TAVYYC | TVLHQDWLNGKE | | MNSLR | | YYCQQ | PEDFA | NO: 415) |
| | | ATGNPY | YKCKVSNKALPA | | AEDTA | | GYNAPI | TYYCQ | |
| | | YTNGFN | PIEKTISKAKGQPR | | VYYCA | | TFGGGT | NNYYV | |
| | | SWGQGT | EPQVYTLPPSRDE | | SEYGLA | | KVEIK | GPVSY | |
| | | LVTVSS | LTKNQVSLTCLV | | FDQWG | | (SEQ ID | AFGGG | |
| | | (SEQ ID | KGFYPSDIAVEWE | | QGTLV | | NO: 44) | TKVEI | |
| | | NO: 514) | SNGQPENNYKTTP | | TVSS | | | K (SEQ | |
| | | | PVLDSDGSFFLYS | | (SEQ ID | | | ID NO: | |
| | | | KLTVDKSRWQQG | | NO: 84) | | | 515) | |
| | | | NVFSCSVMHEAL | | | | | | |
| | | | HNHYTQKSLSLSP | | | | | | |
| | | | G (SEQ ID NO: 513) | | | | | | |
| TA33 | MGWS | QVQLVQ | ASTKGPSVFPLAP | GGGGS | EVQLV | GGGGS | DIQMT | DIQMT | RTVAAPSV |
| | CIILFL | SGAEVK | SSKSTSGGTAALG | GGGGS | ESGGGL | GGGGS | QSPSSL | QSPSSL | FIFPPSDEQ |
| | VATA | KPGASV | CLVKDYFPEPVTV | GGGGS | VQPGRS | GGGGS | SASVG | SASVG | LKSGTASV |
| | TGVH | KVSCKV | SWNSGALTSGVH | (SEQ ID | LRLSCA | GGGGS | DRVTIT | DRVTIT | VCLLNNF |
| | S (SEQ | SGYSLS | TFPAVLQSSGLYS | NO: 3) | ASGLTF | (SEQ ID | CRASQ | CQASQ | YPREAKV |
| | ID NO: | KYDMS | LSSVVTVPSSSLG | | SNHGM | NO: 4) | DIAGW | SPNNL | QWKVDNA |
| | 588) | WVRQA | TQTYICNVNHKPS | | HWVRQ | | LAWYQ | LAWY | LQSGNSQE |
| | | PGKGLE | NTKVDKKVEPKS | | APGKG | | QKPGK | QQKPG | SVTEQDSK |
| | | WMGIIY | CDKTHTCPPCPAP | | LEWVS | | APKLLI | KAPKL | DSTYSLSS |
| | | TSGYTD | EAAGAPSVFLFPP | | AISSSA | | YDASTL | LIYGAS | TLTLSKAD |
| | | YAQKFQ | KPKDTLMISRTPE | | DIVDYA | | QGGVP | DLPSG | YEKHKVY |
| | | GRVTMT | VTCVVVDVSHED | | DSVEG | | SRFSGS | VPSRFS | ACEVTHQ |
| | | EDTSTD | PEVKFNWYVDGV | | RFTISR | | GSGTDF | GSGSG | GLSSPVTK |
| | | TAYMEL | EVHNAKTKPREE | | DNAKN | | TLTISSL | TDFTL | SFNRGEC |
| | | SSLRSED | QYNSTYRVVSVL | | SLYLQ | | QPEDV | TISSLQ | (SEQ ID |
| | | TAVYYC | TVLHQDWLNGKE | | MNSLR | | ATYYC | PEDFA | NO: 415) |
| | | ATGNPY | YKCKVSNKALPA | | AEDTA | | QQSYT | TYYCQ | |
| | | YTNGFN | PIEKTISKAKGQPR | | VYYCA | | APLNFG | NNYYV | |
| | | SWGQGT | EPQVYTLPPSRDE | | SEGVPY | | GGTKV | GPVSY | |
| | | LVTVSS | LTKNQVSLTCLV | | GMDV | | EIK | AFGGG | |
| | | (SEQ ID | KGFYPSDIAVEWE | | WGQGT | | (SEQ ID | TKVEI | |
| | | NO: 514) | SNGQPENNYKTTP | | MVTVS | | NO: 49) | K (SEQ | |
| | | | PVLDSDGSFFLYS | | S (SEQ | | | ID NO: | |
| | | | KLTVDKSRWQQG | | ID NO: | | | | |
| | | | NVFSCSVMHEAL | | | | | | |
| | | | HNHYTQKSLSLSP | | 85) | | | 515) | |
| | | | G (SEQ ID NO: 513) | | | | | | |
| TA34 | MGWS | QVQLVQ | ASTKGPSVFPLAP | GGGGS | EVQLV | GGGGS | DIQMT | DIQMT | RTVAAPSV |
| | CIILFL | SGAEVK | SSKSTSGGTAALG | GGGGS | ESGGGL | GGGGS | QSPSSL | QSPSSL | FIFPPSDEQ |
| | VATA | KPGASV | CLVKDYFPEPVTV | GGGGS | VQPGRS | GGGGS | SASVG | SASVG | LKSGTASV |
| | TGVH | KVSCKV | SWNSGALTSGVH | (SEQ ID | LRLSCA | GGGGS | DRVTIT | DRVTIT | VCLLNNF |
| | S (SEQ | SGYSLS | TFPAVLQSSGLYS | NO: 3) | ASGSTL | (SEQ ID | CRASQ | CQASQ | YPREAKV |
| | ID NO: | KYDMS | LSSVVTVPSSSLG | | SSYGM | NO: 4) | GIGSYL | SPNNL | QWKVDNA |
| | 588) | WVRQA | TQTYICNVNHKPS | | HWVRQ | | AWYQQ | LAWY | LQSGNSQE |
| | | PGKGLE | NTKVDKKVEPKS | | APGKG | | KPGKA | QQKPG | SVTEQDSK |
| | | WMGIIY | CDKTHTCPPCPAP | | LEWVS | | PKLLIY | KAPKL | DSTYSLSS |
| | | TSGYTD | EAAGAPSVFLFPP | | AISYDA | | EASRLE | LIYGAS | TLTLSKAD |
| | | YAQKFQ | KPKDTLMISRTPE | | STIDYA | | SGVPSR | DLPSG | YEKHKVY |
| | | GRVTMT | VTCVVVDVSHED | | DSVEG | | FSGSGS | VPSRFS | ACEVTHQ |
| | | EDTSTD | PEVKFNWYVDGV | | RFTISR | | GTDFTL | GSGSG | GLSSPVTK |
| | | TAYMEL | EVHNAKTKPREE | | DNAKN | | TISSLQP | TDFTL | SFNRGEC |
| | | SSLRSED | QYNSTYRVVSVL | | SLYLQ | | EDVAT | TISSLQ | (SEQ ID |
| | | TAVYYC | TVLHQDWLNGKE | | MNSLR | | YYCQQ | PEDFA | NO: 415) |
| | | ATGNPY | YKCKVSNKALPA | | AEDTA | | GYNAPI | TYYCQ | |
| | | YTNGFN | PIEKTISKAKGQPR | | VYYCA | | TFGGGT | NNYYV | |
| | | SWGQGT | EPQVYTLPPSRDE | | SEGVPY | | KVEIK | GPVSY | |
| | | LVTVSS | LTKNQVSLTCLV | | GMDV | | (SEQ ID | AFGGG | |
| | | (SEQ ID | KGFYPSDIAVEWE | | WGQGT | | NO: 44) | TKVEI | |
| | | NO: 514) | SNGQPENNYKTTP | | MVTVS | | | K (SEQ | |
| | | | PVLDSDGSFFLYS | | S (SEQ | | | ID NO: | |
| | | | KLTVDKSRWQQG | | ID NO: | | | 515) | |
| | | | NVFSCSVMHEAL | | 86) | | | | |
| | | | HNHYTQKSLSLSP | | | | | | |
| | | | G (SEQ ID NO: 513) | | | | | | |
| TA35 | MGWS | QVQLVQ | ASTKGPSVFPLAP | GGGGS | QVQLQ | GGGGS | EIVMTQ | DIQMT | RTVAAPSV |
| | CIILFL | SGAEVK | SSKSTSGGTAALG | GGGGS | ESGPGL | GGGGS | SPATLS | QSPSSL | FIFPPSDEQ |
| | VATA | KPGASV | CLVKDYFPEPVTV | GGGGS | VKPSQT | GGGGS | LSPGER | SASVG | LKSGTASV |
| | TGVH | KVSCKV | SWNSGALTSGVH | (SEQ ID | LSLTCT | GGGGS | ATLSCR | DRVTIT | VCLLNNF |
| | S (SEQ | SGYSLS | TFPAVLQSSGLYS | NO: 3) | VSGESF | (SEQ ID | ASQTIG | CQASQ | YPREAKV |
| | ID NO: | KYDMS | LSSVVTVPSSSLG | | SDFYW | NO: 4) | TNLAW | SPNNL | QWKVDNA |
| | 588) | WVRQA | TQTYICNVNHKPS | | SWIRQP | | YQQKP | LAWY | LQSGNSQE |
| | | PGKGLE | NTKVDKKVEPKS | | PGKGLE | | GQAPR | QQKPG | SVTEQDSK |
| | | WMGIIY | CDKTHTCPPCPAP | | WIGYID | | LLIYDA | KAPKL | DSTYSLSS |
| | | TSGYTD | EAAGAPSVFLFPP | | HTGSTD | | STRAN | LIYGAS | TLTLSKAD |
| | | YAQKFQ | KPKDTLMISRTPE | | YNPSLK | | GIPARF | DLPSG | YEKHKVY |
| | | GRVTMT | VTCVVVDVSHED | | SRVTM | | SGSGSG | VPSRFS | ACEVTHQ |
| | | EDTSTD | PEVKFNWYVDGV | | SVDTSK | | TDFTLT | GSGSG | GLSSPVTK |
| | | TAYMEL | EVHNAKTKPREE | | NQFSLK | | ISSLEPE | TDFTL | SFNRGEC |
| | | SSLRSED | QYNSTYRVVSVL | | VNSVT | | DFAVY | TISSLQ | (SEQ ID |
| | | TAVYYC | TVLHQDWLNGKE | | AADTA | | YCQQY | PEDFA | NO: 415) |
| | | ATGNPY | YKCKVSNKALPA | | VYYCA | | AAPPLT | TYYCQ | |
| | | YTNGFN | PIEKTISKAKGQPR | | GDKYA | | FGGGT | NNYYV | |
| | | SWGQGT | EPQVYTLPPSRDE | | DGFDV | | KVEIK | GPVSY | |
| | | LVTVSS | LTKNQVSLTCLV | | WGQGT | | (SEQ ID | AFGGG | |
| | | (SEQ ID | KGFYPSDIAVEWE | | MVTVS | | NO: 50) | TKVEI | |
| | | NO: 514) | SNGQPENNYKTTP | | S (SEQ | | | K (SEQ | |
| | | | PVLDSDGSFFLYS | | ID NO: | | | ID NO: | |
| | | | KLTVDKSRWQQG | | 87) | | | 515) | |
| | | | NVFSCSVMHEAL | | | | | | |
| | | | HNHYTQKSLSLSP | | | | | | |
| | | | G (SEQ ID NO: 513) | | | | | | |
| TA36 | MGWS | QVQLVQ | ASTKGPSVFPLAP | GGGGS | QVQLQ | GGGGS | EIVMTQ | DIQMT | RTVAAPSV |
| | CIILFL | SGAEVK | SSKSTSGGTAALG | GGGGS | ESGPGL | GGGGS | SPATLS | QSPSSL | FIFPPSDEQ |
| | VATA | KPGASV | CLVKDYFPEPVTV | GGGGS | VKPSQT | GGGGS | LSPGER | SASVG | LKSGTASV |
| | TGVH | KVSCKV | SWNSGALTSGVH | (SEQ ID | LSLTCT | GGGGS | ATLSCR | DRVTIT | VCLLNNF |
| | S (SEQ | SGYSLS | TFPAVLQSSGLYS | NO: 3) | VSGFSL | (SEQ ID | ASQSIR | CQASQ | YPREAKV |
| | ID NO: | KYDMS | LSSVVTVPSSSLG | | STSGVR | NO: 4) | SDLAW | SPNNL | QWKVDNA |
| | 588) | WVRQA | TQTYICNVNHKPS | | WSWIR | | YQQKP | LAWY | LQSGNSQE |
| | | PGKGLE | NTKVDKKVEPKS | | QPPGK | | GQAPR | QQKPG | SVTEQDSK |
| | | WMGIIY | CDKTHTCPPCPAP | | GLEWIG | | LLIYDA | KAPKL | DSTYSLSS |
| | | TSGYTD | EAAGAPSVFLFPP | | YINPSG | | SHRPAG | LIYGAS | TLTLSKAD |
| | | YAQKFQ | KPKDTLMISRTPE | | TTDYNP | | IPARFS | DLPSG | YEKHKVY |
| | | GRVTMT | VTCVVVDVSHED | | SLKSRV | | GSGSGT | VPSRFS | ACEVTHQ |
| | | EDTSTD | PEVKFNWYVDGV | | TMSVD | | DFTLTI | GSGSG | GLSSPVTK |
| | | TAYMEL | EVHNAKTKPREE | | TSKNQF | | SSLEPE | TDFTL | SFNRGEC |
| | | SSLRSED | QYNSTYRVVSVL | | SLKVNS | | DFAVY | TISSLQ | (SEQ ID |
| | | TAVYYC | TVLHQDWLNGKE | | VTAAD | | YCQQY | PEDFA | NO: 415) |
| | | ATGNPY | YKCKVSNKALPA | | TAVYY | | GSVPRP | TYYCQ | |
| | | YTNGFN | PIEKTISKAKGQPR | | CARGG | | TFGGGT | NNYYV | |
| | | SWGQGT | EPQVYTLPPSRDE | | GYCSG | | KVEIK | GPVSY | |
| | | LVTVSS | LTKNQVSLTCLV | | GSCYD | | (SEQ ID | AFGGG | |
| | | (SEQ ID | KGFYPSDIAVEWE | | WYFDL | | NO: 51) | TKVEI | |
| | | NO: 514) | SNGQPENNYKTTP | | WGRGT | | | K (SEQ | |
| | | | PVLDSDGSFFLYS | | LVTVSS | | | ID NO: | |
| | | | KLTVDKSRWQQG | | (SEQ ID | | | 515) | |
| | | | NVFSCSVMHEAL | | NO: 88) | | | | |
| | | | HNHYTQKSLSLSP | | | | | | |
| | | | G (SEQ ID NO: 513) | | | | | | |
| TA37 | MGWS | QVQLVQ | ASTKGPSVFPLAP | GGGGS | QVQLQ | GGGGS | EIVMTQ | DIQMT | RTVAAPSV |
| | CIILFL | SGAEVK | SSKSTSGGTAALG | GGGGS | ESGPGL | GGGGS | SPATLS | QSPSSL | FIFPPSDEQ |
| | VATA | KPGASV | CLVKDYFPEPVTV | GGGGS | VKPSQT | GGGGS | LSPGER | SASVG | LKSGTASV |
| | TGVH | KVSCKV | SWNSGALTSGVH | (SEQ ID | LSLTCT | GGGGS | ATLSCR | DRVTIT | VCLLNNF |
| | S (SEQ | SGYSLS | TFPAVLQSSGLYS | NO: 3) | VSGFSL | (SEQ ID | ASQSIR | CQASQ | YPREAKV |
| | ID NO: | KYDMS | LSSVVTVPSSSLG | | STSGVR | NO: 4) | SDLAW | SPNNL | QWKVDNA |
| | 588) | WVRQA | TQTYICNVNHKPS | | WSWIR | | YQQKP | LAWY | LQSGNSQE |
| | | PGKGLE | NTKVDKKVEPKS | | QPPGK | | GQAPR | QQKPG | SVTEQDSK |
| | | WMGIIY | CDKTHTCPPCPAP | | GLEWIG | | LLIYDA | KAPKL | DSTYSLSS |
| | | TSGYTD | EAAGAPSVFLFPP | | YINPSG | | TSRAA | LIYGAS | TLTLSKAD |
| | | YAQKFQ | KPKDTLMISRTPE | | TTDYNP | | GIPARF | DLPSG | YEKHKVY |
| | | GRVTMT | VTCVVVDVSHED | | SLKSRV | | SGSGSG | VPSRFS | ACEVTHQ |
| | | EDTSTD | PEVKFNWYVDGV | | TMSVD | | TDFTLT | GSGSG | GLSSPVTK |
| | | TAYMEL | EVHNAKTKPREE | | TSKNQF | | ISSLEPE | TDFTL | SFNRGEC |
| | | SSLRSED | QYNSTYRVVSVL | | SLKVNS | | DFAVY | TISSLQ | (SEQ ID |
| | | TAVYYC | TVLHQDWLNGKE | | VTAAD | | YCQEY | PEDFA | NO: 415) |
| | | ATGNPY | YKCKVSNKALPA | | TAVYY | | GSAPLT | TYYCQ | |
| | | YTNGFN | PIEKTISKAKGQPR | | CARGG | | FGGGT | NNYYV | |
| | | SWGQGT | EPQVYTLPPSRDE | | GYCSG | | KVEIK | GPVSY | |
| | | LVTVSS | LTKNQVSLTCLV | | GSCYD | | (SEQ ID | AFGGG | |
| | | (SEQ ID | KGFYPSDIAVEWE | | WYFDL | | NO: 52) | TKVEI | |
| | | NO: 514) | SNGQPENNYKTTP | | WGRGT | | | K (SEQ | |
| | | | PVLDSDGSFFLYS | | LVTVSS | | | ID NO: | |
| | | | KLTVDKSRWQQG | | (SEQ ID | | | 515) | |
| | | | NVFSCSVMHEAL | | NO: 88) | | | | |
| | | | HNHYTQKSLSLSP | | | | | | |
| | | | G (SEQ ID NO: 513) | | | | | | |
| TA38 | MGWS | QVQLVQ | ASTKGPSVFPLAP | GGGGS | QVQLQ | GGGGS | EIVMTQ | DIQMT | RTVAAPSV |
| | CIILFL | SGAEVK | SSKSTSGGTAALG | GGGGS | ESGPGL | GGGGS | SPATLS | QSPSSL | FIFPPSDEQ |
| | VATA | KPGASV | CLVKDYFPEPVTV | GGGGS | VKPSQT | GGGGS | LSPGER | SASVG | LKSGTASV |
| | TGVH | KVSCKV | SWNSGALTSGVH | (SEQ ID | LSLTCT | GGGGS | ATLSCR | DRVTIT | VCLLNNF |
| | S (SEQ | SGYSLS | TFPAVLQSSGLYS | NO: 3) | VSGESF | (SEQ ID | ASMGV | CQASQ | YPREAKV |
| | ID NO: | KYDMS | LSSVVTVPSSSLG | | SGYSW | NO: 4) | SSNLA | SPNNL | QWKVDNA |
| | 588) | WVRQA | TQTYICNVNHKPS | | SWIRQP | | WYQQK | LAWY | LQSGNSQE |
| | | PGKGLE | NTKVDKKVEPKS | | PGKGLE | | PGQAPR | QQKPG | SVTEQDSK |
| | | WMGIIY | CDKTHTCPPCPAP | | WIGYIT | | LLIYDA | KAPKL | DSTYSLSS |
| | | TSGYTD | EAAGAPSVFLFPP | | HSGTID | | SNRAA | LIYGAS | TLTLSKAD |
| | | YAQKFQ | KPKDTLMISRTPE | | YNPSLK | | GIPARF | DLPSG | YEKHKVY |
| | | GRVTMT | VTCVVVDVSHED | | SRVTM | | SGSGSG | VPSRFS | ACEVTHQ |
| | | EDTSTD | PEVKFNWYVDGV | | SVDTSK | | TDFTLT | GSGSG | GLSSPVTK |
| | | TAYMEL | EVHNAKTKPREE | | NQFSLK | | ISSLEPE | TDFTL | SFNRGEC |
| | | SSLRSED | QYNSTYRVVSVL | | VNSVT | | DFAVY | TISSLQ | (SEQ ID |
| | | TAVYYC | TVLHQDWLNGKE | | AADTA | | YCQQY | PEDFA | NO: 415) |
| | | ATGNPY | YKCKVSNKALPA | | VYYCA | | AEGPLT | TYYCQ | |
| | | YTNGFN | PIEKTISKAKGQPR | | KPLDFG | | FGGGT | NNYYV | |
| | | SWGQGT | EPQVYTLPPSRDE | | DYKDA | | KVEIK | GPVSY | |
| | | LVTVSS | LTKNQVSLTCLV | | FDIWG | | (SEQ ID | AFGGG | |
| | | (SEQ ID | KGFYPSDIAVEWE | | QGTMV | | NO: 53) | TKVEI | |
| | | NO: 514) | SNGQPENNYKTTP | | TVSS | | | K (SEQ | |
| | | | PVLDSDGSFFLYS | | (SEQ ID | | | ID NO: | |
| | | | KLTVDKSRWQQG | | NO: 89) | | | 515) | |
| | | | NVFSCSVMHEAL | | | | | | |
| | | | HNHYTQKSLSLSP | | | | | | |
| | | | G (SEQ ID NO: 513) | | | | | | |
| TA39 | MGWS | QVQLVQ | ASTKGPSVFPLAP | GGGGS | EVQLV | GGGGS | DIQMT | DIQMT | RTVAAPSV |
| | CIILFL | SGAEVK | SSKSTSGGTAALG | GGGGS | ESGGGL | GGGGS | QSPSSL | QSPSSL | FIFPPSDEQ |
| | VATA | KPGASV | CLVKDYFPEPVTV | GGGGS | VQPGRS | GGGGS | SASVG | SASVG | LKSGTASV |
| | TGVH | KVSCKV | SWNSGALTSGVH | (SEQ ID | LRLSCA | GGGGS | DRVTIT | DRVTIT | VCLLNNF |
| | S (SEQ | SGYSLS | TFPAVLQSSGLYS | NO: 3) | ASGSTL | (SEQ ID | CRASQ | CQASQ | YPREAKV |
| | ID NO: | KYDMS | LSSVVTVPSSSLG | | SSYGM | NO: 4) | GIGSYL | SPNNL | QWKVDNA |
| | 588) | WVRQA | TQTYICNVNHKPS | | HWVRQ | | AWYQQ | LAWY | LQSGNSQE |
| | | PGKGLE | NTKVDKKVEPKS | | APGKG | | KPGKA | QQKPG | SVTEQDSK |
| | | WMGIIY | CDKTHTCPPCPAP | | LEWVS | | PKLLIY | KAPKL | DSTYSLSS |
| | | TSGYTD | EAAGAPSVFLFPP | | AISYDA | | EASRLE | LIYGAS | TLTLSKAD |
| | | YAQKFQ | KPKDTLMISRTPE | | STIDYA | | SGVPSR | DLPSG | YEKHKVY |
| | | GRVTMT | VTCVVVDVSHED | | DSVEG | | FSGSGS | VPSRFS | ACEVTHQ |
| | | EDTSTD | PEVKFNWYVDGV | | RFTISR | | GTDFTL | GSGSG | GLSSPVTK |
| | | TAYMEL | EVHNAKTKPREE | | DNAKN | | TISSLQP | TDFTL | SFNRGEC |
| | | SSLRSED | QYNSTYRVVSVL | | SLYLQ | | EDVAT | TISSLQ | (SEQ ID |
| | | TAVYYC | TVLHQDWLNGKE | | MNSLR | | YYCQQ | PEDFA | NO: 415) |
| | | ATGNPY | YKCKVSNKALPA | | AEDTA | | GYNAPI | TYYCQ | |
| | | YTNGFN | PIEKTISKAKGQPR | | VYYCA | | TFGGGT | NNYYV | |
| | | SWGQGT | EPQVYTLPPSRDE | | RDGISG | | KVEIK | GPVSY | |
| | | LVTVSS | LTKNQVSLTCLV | | IDYWG | | (SEQ ID | AFGGG | |
| | | (SEQ ID | KGFYPSDIAVEWE | | QGTLV | | NO: 44) | TKVEI | |
| | | NO: 514) | SNGQPENNYKTTP | | TVSS | | | K (SEQ | |
| | | | PVLDSDGSFFLYS | | (SEQ ID | | | ID NO: | |
| | | | KLTVDKSRWQQG | | NO: 90) | | | 515) | |
| | | | NVFSCSVMHEAL | | | | | | |
| | | | HNHYTQKSLSLSP | | | | | | |
| | | | G (SEQ ID NO: 513) | | | | | | |

For clarity, the tables provided herein, such as the table above, include the signal peptide sequence, which can be used to facilitate the expression of the molecule, but is removed during a post-translational process. Thus, the processed polypeptides that form the bi-directional molecule that can bind to PD-1 and/or FcγRIIβ would not have, or do not need, the signal peptide present when administered to a subject.

In some embodiments, a bispecific antibody comprising an anti-PD-1 antibody and an anti-FcγRIIβ antibody is provided. In some embodiments, a bispecific antibody comprising an anti-PD-1 antibody in Fab format and an anti-FcγRIIβ antibody in scFv format is provided. In some embodiments, a bispecific antibody comprising an anti-PD-1 antibody in scFv format and an anti-FcγRIIβ antibody in Fab format is provided. In some embodiments, a bispecific antibody comprises an anti-PD-1 antibody, an Fc molecule, and an anti-FcγRIIβ antibody. In some embodiments, a bispecific antibody comprises an anti-PD-1 antibody in Fab format, an Fc molecule, and an anti-FcγRIIβ antibody in scFv format. In some embodiments, a bispecific antibody comprises an anti-PD-1 antibody in scFv format, an Fc molecule, and an anti-FcγRIIβ antibody in Fab format. In some embodiments, the bispecific antibody is as provided in Table 14. In some embodiments, the bispecific antibody as provided in Table 14 further comprises a Ck. In some embodiments, the bispecific antibody as provided in Table 14 further comprises a Ck having an amino acid sequence of SEQ ID NO: 415.

TABLE 14

| | VH | VL | | C- | SCFv VH | SCFv | SCFv VL |
|---|---|---|---|---|---|---|---|
| Molecule | (anti- | (anti- | | terminal | (anti- | Linker | (anti- |
| ID | PD1) | PD1) | Fc | linker | FcγRIIb) | | FcγRIIb) |
| TA40 | EVQLLESG | EIVMTQS | ASTKGPSVFPLAPSSK | GGGGSGGG | EVQLVESG | GGGGSG | DIVMTQSPL |
| | GGLVQPGG | PATLSVS | STSGGTAALGCLVKDY | GSGGGGSG | GGLVQPGG | GGGSGG | SLPVTPGEP |
| | SLRLSCAA | PGERATL | FPEPVTVSWNSGALTS | GGGS (SEQ | SLRLSCAA | GGSGGG | ASISCRSSR |
| | SGFTFSSD | SCRASQS | GVHTFPAVLQSSGLYS | ID NO: 4) | SRFTFSDY | GS (SEQ | SLVHGSGDN |
| | AMNWVRQA | VSSNLAW | LSSVVTVPSSSLGTQT | | HMSWVRQA | ID NO: 4) | YLHWYLQKP |
| | PGKGLEWY | YQQKPGQ | YICNVNHKPSNTKVDK | | PGKGLELV | | GQSPQLLIY |
| | STIYSGGS | APRLLIY | KVEPKSCDKTHTCPPC | | ASIDTEGK | | MASNRAPGV |
| | TYYADSVK | GASTRAT | PAPEAAGAPSVFLFPP | | TYYPDSVK | | PDRFSGSGS |
| | GRFTISRD | GIPARFS | KPKDTLMISRTPEVTC | | GRFTISRD | | GTDFTLKIS |
| | NSKNTLYL | GSGSGTE | VVVDVSHEDPEVKFNW | | NAKNSLYL | | RVEAEDVGV |
| | QMNSLRAE | FTLTISS | YVDGVEVHNAKTKPRE | | QMNSLRAE | | YYCMQALRA |
| | DTAVYYCA | LQSEDFA | EQYNSTYRVVSVLTVL | | DTAVYYCA | | PFSFGGGTK |
| | RGGNFYNY | VYYCQQY | HQDWLNGKEYKCKVSN | | RDVGDWYF | | VEIK (SEQ |
| | FDYWGQGT | NNWPPWT | KALPAPIEKTISKAKG | | DLWGRGTL | | ID NO: 42) |
| | LVTVSS | FGQGTKV | QPREPQVYTLPPSRDE | | VTVSS | | |
| | (SEQ ID | EIK | LTKNQVSLTCLVKGFY | | (SEQ ID NO: | | |
| | NO: 136) | (SEQ ID | PSDIAVEWESNGQPEN | | 78) | | |
| | | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ ID | | | | |
| | | | NO: 513) | | | | |
| TA 41 | EVQLLESG | EIVMTQS | ASTKGPSVFPLAPSSK | GGGGSGGG | EVQLVESG | GGGGSG | DIQMTQSPS |
| | GGLVQPGG | PATLSVS | STSGGTAALGCLVKDY | GSGGGGSG | GGLVQPGR | GGGSGG | SLSASVGDR |
| | SLRLSCAA | PGERATL | FPEPVTVSWNSGALTS | GGGS (SEQ | SLRLSCAA | GGSGGG | VTITCRASQ |
| | SGFTFSSD | SCRASQS | GVHTFPAVLQSSGLYS | ID NO: 4) | SGSTLSSY | GS (SEQ | GIGSYLAWY |
| | AMNWVRQA | VSSNLAW | LSSVVTVPSSSLGTQT | | GMHWVRQA | ID NO: 4) | QQKPGKAPK |
| | PGKGLEWV | YQQKPGQ | YICNVNHKPSNTKVDK | | PGKGLEWV | | LLIYEASRL |
| | STIYSGGS | APRLLIY | KVEPKSCDKTHTCPPC | | SAISYDAS | | ESGVPSRFS |
| | TYYADSVK | GASTRAT | PAPEAAGAPSVFLFPP | | TIDYADSV | | GSGSGTDFT |
| | GRFTISRD | GIPARFS | KPKDTLMISRTPEVTC | | EGRFTISR | | LTISSLQPE |
| | NSKNTLYL | GSGSGTE | VVVDVSHEDPEVKFNW | | DNAKNSLY | | DVATYYCQQ |
| | QMNSLRAE | FTLTISS | YVDGVEVHNAKTKPRE | | LQMNSLRA | | GYNAPITFG |
| | DTAVYYCA | LQSEDFA | EQYNSTYRVVSVLTVL | | EDTAVYYC | | GGTKVEIK |
| | RGGNFYNY | VYYCQQY | HQDWLNGKEYKCKVSN | | ARDGISGI | | (SEQ ID NO: |
| | FDYWGQGT | NNWPPWT | KALPAPIEKTISKAKG | | DYWGQGTL | | 44) |
| | LVTVSS | FGQGTKV | QPREPQVYTLPPSRDE | | VTVSS | | |
| | (SEQ ID | EIK | LTKNQVSLTCLVKGFY | | (SEQ ID NO: | | |
| | NO: 136) | (SEQ ID | PSDIAVEWESNGQPEN | | 90) | | |
| | | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ ID | | | | |
| | | | NO: 513) | | | | |
| TA42 | EVQLLESG | EIVMTQS | ASTKGPSVFPLAPSSK | GGGGSGGG | EVQLVESG | GGGGSG | DIVMTQSPL |
| | GGLVQPGG | PATLSVS | STSGGTAALGCLVKDY | GSGGGGSG | GGLVQPGG | GGGSGG | SLPVTPGEP |
| | SLRLSCAA | PGERATL | FPEPVTVSWNSGALTS | GGGS (SEQ | SLRLSCAA | GGSGGG | ASISCRSSR |
| | SGFTFSDH | SCRASQS | GVHTFPAVLQSSGLYS | ID NO: 4) | SRFTFSDY | GS (SEQ | SLVHGSGDN |
| | YMSWVRQA | VSSNLAW | LSSVVTVPSSSLGTQT | | HMSWVRQA | ID NO: 4) | YLHWYLQKP |
| | PGKGLEWV | YQQKPGQ | YICNVNHKPSNTKVDK | | PGKGLELV | | GQSPQLLIY |
| | STISSGGN | APRLLIY | KVEPKSCDKTHTCPPC | | ASIDTEGK | | MASNRAPGV |
| | YIYYADSV | GASTRAT | PAPEAAGAPSVFLFPP | | TYYPDSVK | | PDRFSGSGS |
| | KGRFTISR | GIPARFS | KPKDTLMISRTPEVTC | | GRFTISRD | | GTDFTLKIS |
| | DSSKNTLY | GSGSGTE | VVVDVSHEDPEVKFNW | | NAKNSLYL | | RVEAEDVGV |
| | LQMNSLRA | FTLTISS | YVDGVEVHNAKTKPRE | | QMNSLRAE | | YYCMQALRA |
| | EDTAVYYC | LQSEDFA | EQYNSTYRVVSVLTVL | | DTAVYYCA | | PFSFGGGTK |
| | ARILKNGK | VYYCQQY | HQDWLNGKEYKCKVSN | | RDVGDWYF | | VEIK (SEQ |
| | YIYYFDYW | NNWPPWT | KALPAPIEKTISKAKG | | DLWGRGTL | | ID NO: 42) |
| | GQGTLVTV | FGQGTKV | QPREPQVYTLPPSRDE | | VTVSS | | |
| | SS (SEQ | EIK | LTKNQVSLTCLVKGFY | | (SEQ ID NO: | | |
| | ID NO: | (SEQ ID | PSDIAVEWESNGQPEN | | 78) | | |
| | 156) | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ ID | | | | |
| | | | NO: 513) | | | | |
| TA43 | EVQLLESG | EIVMTQS | ASTKGPSVFPLAPSSK | GGGGSGGG | EVQLVESG | GGGGSG | DIQMTQSPS |
| | GGLVQPGG | PATLSVS | STSGGTAALGCLVKDY | GSGGGGSG | GGLVQPGR | GGGSGG | SLSASVGDR |
| | SLRLSCAA | PGERATL | FPEPVTVSWNSGALTS | GGGS (SEQ | SLRLSCAA | GGSGGG | VTITCRASQ |
| | SGFTFSDH | SCRASQS | GVHTFPAVLQSSGLYS | ID NO: 4) | SGSTLSSY | GS (SEQ | GIGSYLAWY |
| | YMSWVRQA | VSSNLAW | LSSVVTVPSSSLGTQT | | GMHWVRQA | ID NO: 4) | QQKPGKAPK |
| | PGKGLEWV | YQQKPGQ | YICNVNHKPSNTKVDK | | PGKGLEWV | | LLIYEASRL |
| | STISSGGN | APRLLIY | KVEPKSCDKTHTCPPC | | SAISYDAS | | ESGVPSRFS |
| | YIYYADSV | GASTRAT | PAPEAAGAPSVFLFPP | | TIDYADSV | | GSGSGTDFT |
| | KGRFTISR | GIPARFS | KPKDTLMISRTPEVTC | | EGRFTISR | | LTISSLQPE |
| | DSSKNTLY | GSGSGTE | VVVDVSHEDPEVKFNW | | DNAKNSLY | | DVATYYCQQ |
| | LQMNSLRA | FTLTISS | YVDGVEVHNAKTKPRE | | LQMNSLRA | | GYNAPITFG |
| | EDTAVYYC | LQSEDFA | EQYNSTYRVVSVLTVL | | EDTAVYYC | | GGTKVEIK |
| | ARILKNGK | VYYCQQY | HQDWLNGKEYKCKVSN | | ARDGISGI | | (SEQ ID NO: |
| | YIYYFDYW | NNWPPWT | KALPAPIEKTISKAKG | | DYWGQGTL | | 44) |
| | GQGTLVTV | FGQGTKV | QPREPQVYTLPPSRDE | | VTVSS | | |
| | SS (SEQ | EIK | LTKNQVSLTCLVKGFY | | (SEQ ID NO: | | |
| | ID NO: | (SEQ ID | PSDIAVEWESNGQPEN | | 90) | | |
| | 156) | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ ID | | | | |
| | | | NO: 513) | | | | |
| TA44 | EVQLLESG | QSVLTQP | ASTKGPSVFPLAPSSK | GGGGSGGG | EVQLVESG | GGGGSG | DIVMTQSPL |
| | GAFVQPGG | PSASGAP | STSGGTAALGCLVKDY | GSGGGGSG | GGLVQPGG | GGGSGG | SLPVTPGEP |
| | SLRLSCAA | GQRVTIS | FPEPVTVSWNSGALTS | GGGS (SEQ | SLRLSCAA | GGSGGG | ASISCRSSR |
| | SGFTFSDY | CSGSYSN | GVHTFPAVLQSSGLYS | ID NO: 4) | SRFTFSDY | GS (SEQ | SLVHGSGDN |
| | YMSWVRQA | IGNSYVS | LSSVVTVPSSSLGTQT | | HMSWVRQA | ID NO: 4) | YLHWYLQKP |
| | PGKGLEWV | WYQQLPG | YICNVNHKPSNTKVDK | | PGKGLELV | | GQSPQLLIY |
| | SVISNSGG | TAPKLLI | KVEPKSCDKTHTCPPC | | ASIDTEGK | | MASNRAPGV |
| | STYYTDSV | YLNSQRP | PAPEAAGAPSVFLFPP | | TYYPDSVK | | PDRFSGSGS |
| | KGRFTISR | SGVPDRF | KPKDTLMISRTPEVTC | | GRFTISRD | | GTDFTLKIS |
| | DNSKNTLY | SGSKSGT | VVVDVSHEDPEVKFNW | | NAKNSLYL | | RVEAEDVGV |
| | LQINSLRA | SASLAIS | YVDGVEVHNAKTKPRE | | QMNSLRAE | | YYCMQALRA |
| | EDTAVYYC | GLQSEDE | EQYNSTYRVVSVLTVL | | DTAVYYCA | | PFSFGGGTK |
| | TKDIGMTY | ADYYCAA | HQDWLNGKEYKCKVSN | | RDVGDWYF | | VEIK (SEQ |
| | FDYWGQGT | WDDLNVW | KALPAPIEKTISKAKG | | DLWGRGTL | | ID NO: 42) |
| | LVTVSS | VFGGGTK | QPREPQVYTLPPSRDE | | VTVSS | | |
| | (SEQ ID | LTVL | LTKNQVSLTCLVKGFY | | (SEQ ID NO: | | |
| | NO: 187) | (SEQ ID | PSDIAVEWESNGQPEN | | 78) | | |
| | | NO: | NYKTTPPVLDSDGSFF | | | | |
| | | 344) | LYSKLTVDKSRWQQGN | | | | |
| | | | VFSCSVMHEALHNHYT | | | | |
| | | | QKSLSLSPG (SEQ ID | | | | |
| | | | NO: 513) | | | | |
| TA45 | EVQLLESG | QSVLTQP | ASTKGPSVFPLAPSSK | GGGGSGGG | EVQLVESG | GGGGSG | DIQMTQSPS |
| | GAFVQPGG | PSASGAP | STSGGTAALGCLVKDY | GSGGGGSG | GGLVQPGR | GGGSGG | SLSASVGDR |
| | SLRLSCAA | GQRVTIS | FPEPVTVSWNSGALTS | GGGS (SEQ | SLRLSCAA | GGSGGG | VTITCRASQ |
| | SGFTFSDY | CSGSYSN | GVHTFPAVLQSSGLYS | ID NO: 4) | SGSTLSSY | GS (SEQ | GIGSYLAWY |
| | YMSWVRQA | IGNSYVS | LSSVVTVPSSSLGTQT | | GMHWVRQA | ID NO: 4) | QQKPGKAPK |
| | PGKGLEWV | WYQQLPG | YICNVNHKPSNTKVDK | | PGKGLEWV | | LLIYEASRL |
| | SVISNSGG | TAPKLLI | KVEPKSCDKTHTCPPC | | SAISYDAS | | ESGVPSRFS |
| | STYYTDSV | YLNSQRP | PAPEAAGAPSVFLFPP | | TIDYADSV | | GSGSGTDFT |
| | KGRFTISR | SGVPDRF | KPKDTLMISRTPEVTC | | EGRFTISR | | LTISSLQPE |
| | DNSKNTLY | SGSKSGT | VVVDVSHEDPEVKFNW | | DNAKNSLY | | DVATYYCQQ |
| | LQINSLRA | SASLAIS | YVDGVEVHNAKTKPRE | | LQMNSLRA | | GYNAPITFG |
| | EDTAVYYC | GLQSEDE | EQYNSTYRVVSVLTVL | | EDTAVYYC | | GGTKVEIK |
| | TKDIGMTY | ADYYCAA | HQDWLNGKEYKCKVSN | | ARDGISGI | | (SEQ ID NO: |
| | FDYWGQGT | WDDLNVW | KALPAPIEKTISKAKG | | DYWGQGTL | | 44) |
| | LVTVSS | VFGGGTK | QPREPQVYTLPPSRDE | | VTVSS | | |
| | (SEQ ID | LTVL | LTKNQVSLTCLVKGFY | | (SEQ ID NO: | | |
| | NO: 187) | (SEQ ID | PSDIAVEWESNGQPEN | | 90) | | |
| | | NO: | NYKTTPPVLDSDGSFF | | | | |
| | | 344) | LYSKLTVDKSRWQQGN | | | | |
| | | | VFSCSVMHEALHNHYT | | | | |
| | | | QKSLSLSPG (SEQ ID | | | | |
| | | | NO: 513) | | | | |
| TA46 | EVQLLESG | EIVMTQS | ASTKGPSVFPLAPSSK | GGGGSGGG | EVQLVESG | GGGGSG | DIVMTQSPL |
| | GGLVQPGG | PATLSVS | STSGGTAALGCLVKDY | GSGGGGSG | GGLVQPGG | GGGSGG | SLPVTPGEP |
| | SLRLSCAA | PGERATL | FPEPVTVSWNSGALTS | GGGS (SEQ | SLRLSCAA | GGSGGG | ASISCRSSR |
| | SGFTFSSD | SCRASQS | GVHTFPAVLQSSGLYS | ID NO: 4) | SRFTFSDY | GS (SEQ | SLVHGSGDN |
| | AMNWVRQA | VSSNLAW | LSSVVTVPSSSLGTQT | | HMSWVRQA | ID NO: 4) | YLHWYLQKP |
| | PGKGLEWV | YQQKPGQ | YICNVNHKPSNTKVDK | | PGKGLELV | | GQSPQLLIY |
| | STIYSGGS | APRLLIY | KVEPKSCDKTHTCPPC | | ASIDTEGK | | MASNRAPGV |
| | TYYADSVK | GASTRAT | PAPELLGEESVFLFPP | | TYYPDSVK | | PDRFSGSGS |
| | GRFTISRD | GIPARFS | KPKDTLMISRTPEVTC | | GRFTISRD | | GTDFTLKIS |
| | NSKNTLYL | GSGSGTE | VVVDVSHEDPEVKFNW | | NAKNSLYL | | RVEAEDVGV |
| | QMNSLRAE | FTLTISS | YVDGVEVHNAKTKPRE | | QMNSLRAE | | YYCMQALRA |
| | DTAVYYCA | LQSEDFA | EQYNSTYRVVSVLTVL | | DTAVYYCA | | PFSFGGGTK |
| | RGGNFYNY | VYYCQQY | HQDWLNGKEYKCKVSN | | RDVGDWYF | | VEIK (SEQ |
| | FDYWGQGT | NNWPPWT | KALPAPIEKTISKAKG | | DLWGRGTL | | ID NO: 42) |
| | LVTVSS | FGQGTKV | QPREPQVYTLPPSRDE | | VTVSS | | |
| | (SEQ ID | EIK | LTKNQVSLTCLVKGFY | | (SEQ ID NO: | | |
| | NO: 136) | (SEQ ID | PSDIAVEWESNGQPEN | | 78) | | |
| | | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ | | | | |
| | | | ID NO: 543) | | | | |
| TA47 | EVQLLESG | EIVMTQS | ASTKGPSVFPLAPSSK | GGGGSGGG | EVQLVESG | GGGGSG | DIVMTQSPL |
| | GGLVQPGG | PATLSVS | STSGGTAALGCLVKDY | GSGGGGSG | GGLVQPGG | GGGSGG | SLPVTPGEP |
| | SLRLSCAA | PGERATL | FPEPVTVSWNSGALTS | GGGS (SEQ | SLRLSCAA | GGSGGG | ASISCRSSR |
| | SGFTFSSD | SCRASQS | GVHTFPAVLQSSGLYS | ID NO: 4) | SRFTFSDY | GS (SEQ | SLVHGSGDN |
| | AMNWVRQA | VSSNLAW | LSSVVTVPSSSLGTQT | | HMSWVRQA | ID NO: 4) | YLHWYLQKP |
| | PGKGLEWV | YQQKPGQ | YICNVNHKPSNTKVDK | | PGKGLELV | | GQSPQLLIY |
| | STIYSGGS | APRLLIY | KVEPKSCDKTHTCPPC | | ASIDTEGK | | MASNRAPGV |
| | TYYADSVK | GASTRAT | PAPELLGDGSAFLFPP | | TYYPDSVK | | PDRFSGSGS |
| | GRFTISRD | GIPARFS | KPKDTLMISRTPEVTC | | GRFTISRD | | GTDFTLKIS |
| | NSKNTLYL | GSGSGTE | VVVDVSHDEPEVKFNW | | NAKNSLYL | | RVEAEDVGV |
| | QMNSLRAE | FTLTISS | YVDGVEVHNAKTKPRE | | QMNSLRAE | | YYCMQALRA |
| | DTAVYYCA | LQSEDFA | EQYNSTYRVVSVLTVL | | DTAVYYCA | | PFSFGGGTK |
| | RGGNFYNY | VYYCQQY | HQDWLNGKEYKCKVSN | | RDVGDWYF | | VEIK (SEQ |
| | FDYWGQGT | NNWPPWT | KALPRPIEKTISKAKG | | DLWGRGTL | | ID NO: 42) |
| | LVTVSS | FGQGTKV | QPREPQVYTLPPSRDE | | VTVSS | | |
| | (SEQ ID | EIK | LTKNQVSLTCLVKGFY | | (SEQ ID NO: | | |
| | NO: 136) | (SEQ ID | PSDIAVEWESNGQPEN | | 78) | | |
| | | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ | | | | |
| | | | ID NO: 544) | | | | |
| TA48 | EVQLLESG | EIVMTQS | ASTKGPSVFPLAPSSK | GGGGSGGG | EVQLVESG | GGGGSG | DIVMTQSPL |
| | GGLVQPGG | PATLSVS | STSGGTAALGCLVKDY | GSGGGGSG | GGLVQPGG | GGGSGG | SLPVTPGEP |
| | SLRLSCAA | PGERATL | FPEPVTVSWNSGALTS | GGGS (SEQ | SLRLSCAA | GGSGGG | ASISCRSSR |
| | SGFTFSSD | SCRASQS | GVHTFPAVLQSSGLYS | ID NO: 4) | SRFTFSDY | GS (SEQ | SLVHGSGDN |
| | AMNWVRQA | VSSNLAW | LSSVVTVPSSSLGTQT | | HMSWVRQA | ID NO: 4) | YLHWYLQKP |
| | PGKGLEWV | YQQKPGQ | YICNVNHKPSNTKVDK | | PGKGLELV | | GQSPQLLIY |
| | STIYSGGS | APRLLIY | KVEPKSCDKTHTCPPC | | ASIDTEGK | | MASNRAPGV |
| | TYYADSVK | GASTRAT | PAPEFEGGPSVFLFPP | | TYYPDSVK | | PDRFSGSGS |
| | GRFTISRD | GIPARFS | KPKDTLMISRTPEVTC | | GRFTISRD | | GTDFTLKIS |
| | NSKNTLYL | GSGSGTE | VVVDVSDEDGEVKFNW | | NAKNSLYL | | RVEAEDVGV |
| | QMNSLRAE | FTLTISS | YVDGVEVHNAKTKPRE | | QMNSLRAE | | YYCMQALRA |
| | DTAVYYCA | LQSEDFA | EQYNSTYRVVSVLTVL | | DTAVYYCA | | PFSFGGGTK |
| | RGGNFYNY | VYYCQQY | HQDWLNGKEYKCKVSN | | RDVGDWYF | | VEIK (SEQ |
| | FDYWGQGT | NNWPPWT | KALAAPIEKTISKAKG | | DLWGRGTL | | ID NO: 42) |
| | LVTVSS | FGQGTKV | QPREPQVYTLPPSRDE | | VTVSS | | |
| | (SEQ ID | EIK | LTKNQVSLTCLVKGFY | | (SEQ ID NO: | | |
| | NO: 136) | (SEQ ID | PSDIAVEWESNGQPEN | | 78) | | |
| | | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ | | | | |
| | | | ID NO: 545) | | | | |
| TA49 | EVQLLESG | EIVMTQS | ASTKGPSVFPLAPSSK | GGGGSGGG | EVQLVESG | GGGGSG | DIVMTQSPL |
| | GGLVQPGG | PATLSVS | STSGGTAALGCLVKDY | GSGGGGSG | GGLVQPGG | GGGSGG | SLPVTPGEP |
| | SLRLSCAA | PGERATL | FPEPVTVSWNSGALTS | GGGS (SEQ | SLRLSCAA | GGSGGG | ASISCRSSR |
| | SGFTFSDH | SCRASQS | GVHTFPAVLQSSGLYS | ID NO: 4) | SRFTFSDY | GS (SEQ | SLVHGSGDN |
| | YMSWVRQA | VSSNLAW | LSSVVTVPSSSLGTQT | | HMSWVRQA | ID NO: 4) | YLHWYLQKP |
| | PGKGLEWV | YQQKPGQ | YICNVNHKPSNTKVDK | | PGKGLELV | | GQSPQLLIY |
| | STISSGGN | APRLLIY | KVEPKSCDKTHTCPPC | | ASIDTEGK | | MASNRAPGV |
| | YIYYADSV | GASTRAT | PAPELLGEESVFLFPP | | TYYPDSVK | | PDRFSGSGS |
| | KGRFTISR | GIPARFS | KPKDTLMISRTPEVTC | | GRFTISRD | | GTDFTLKIS |
| | DSSKNTLY | GSGSGTE | VVVDVSHEDPEVKFNW | | NAKNSLYL | | RVEAEDVGV |
| | LQMNSLRA | FTLTISS | YVDGVEVHNAKTKPRE | | QMNSLRAE | | YYCMQALRA |
| | EDTAVYYC | LQSEDFA | EQYNSTYRVVSVLTVL | | DTAVYYCA | | PFSFGGGTK |
| | ARILKNGK | VYYCQQY | HQDWLNGKEYKCKVSN | | RDVGDWYF | | VEIK (SEQ |
| | YIYYFDYW | NNWPPWT | KALPAPIEKTISKAKG | | DLWGRGTL | | ID NO: 42) |
| | GQGTLVTV | FGQGTKV | QPREPQVYTLPPSRDE | | VTVSS | | |
| | SS (SEQ | EIK | LTKNQVSLTCLVKGFY | | (SEQ ID NO: | | |
| | ID NO: | (SEQ ID | PSDIAVEWESNGQPEN | | 78) | | |
| | 156) | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ | | | | |
| | | | ID NO: 543) | | | | |
| TA50 | EVQLLESG | EIVMTQS | ASTKGPSVFPLAPSSK | GGGGSGGG | EVQLVESG | GGGGSG | DIVMTQSPL |
| | GGLVQPGG | PATLSVS | STSGGTAALGCLVKDY | GSGGGGSG | GGLVQPGG | GGGSGG | SLPVTPGEP |
| | SLRLSCAA | PGERATL | FPEPVTVSWNSGALTS | GGGS (SEQ | SLRLSCAA | GGSGGG | ASISCRSSR |
| | SGFTFSDH | SCRASQS | GVHTFPAVLQSSGLYS | ID NO: 4) | SRFTFSDY | GS (SEQ | SLVHGSGDN |
| | YMSWVRQA | VSSNLAW | LSSVVTVPSSSLGTQT | | HMSWVRQA | ID NO: 4) | YLHWYLQKP |
| | PGKGLEWV | YQQKPGQ | YICNVNHKPSNTKVDK | | PGKGLELV | | GQSPQLLIY |
| | STISSGGN | APRLLIY | KVEPKSCDKTHTCPPC | | ASIDTEGK | | MASNRAPGV |
| | YIYYADSV | GASTRAT | PAPELLGDGSAFLFPP | | TYYPDSVK | | PDRFSGSGS |
| | KGRFTISR | GIPARFS | KPKDTLMISRTPEVTC | | GRFTISRD | | GTDFTLKIS |
| | DSSKNTLY | GSGSGTE | VVVDVSHDEPEVKFNW | | NAKNSLYL | | RVEAEDVGV |
| | LQMNSLRA | FTLTISS | YVDGVEVHNAKTKPRE | | QMNSLRAE | | YYCMQALRA |
| | EDTAVYYC | LQSEDFA | EQYNSTYRVVSVLTVL | | DTAVYYCA | | PFSFGGGTK |
| | ARILKNGK | VYYCQQY | HQDWLNGKEYKCKVSN | | RDVGDWYF | | VEIK (SEQ |
| | YIYYFDYW | NNWPPWT | KALPRPIEKTISKAKG | | DLWGRGTL | | ID NO: 42) |
| | GQGTLVTV | FGQGTKV | QPREPQVYTLPPSRDE | | VTVSS | | |
| | SS (SEQ | EIK | LTKNQVSLTCLVKGFY | | (SEQ ID NO: | | |
| | ID NO: | (SEQ ID | PSDIAVEWESNGQPEN | | 78) | | |
| | 156) | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ | | | | |
| | | | ID NO: 544) | | | | |
| TA51 | EVQLLESG | EIVMTQS | ASTKGPSVFPLAPSSK | GGGGSGGG | EVQLVESG | GGGGSG | DIVMTQSPL |
| | GGLVQPGG | PATLSVS | STSGGTAALGCLVKDY | GSGGGGSG | GGLVQPGG | GGGSGG | SLPVTPGEP |
| | SLRLSCAA | PGERATL | FPEPVTVSWNSGALTS | GGGS (SEQ | SLRLSCAA | GGSGGG | ASISCRSSR |
| | SGFTFSDH | SCRASQS | GVHTFPAVLQSSGLYS | ID NO: 4) | SRFTFSDY | GS (SEQ | SLVHGSGDN |
| | YMSWVRQA | VSSNLAW | LSSVVTVPSSSLGTQT | | HMSWVRQA | ID NO: 4) | YLHWYLQKP |
| | PGKGLEWV | YQQKPGQ | YICNVNHKPSNTKVDK | | PGKGLELV | | GQSPQLLIY |
| | STISSGGN | APRLLIY | KVEPKSCDKTHTCPPC | | ASIDTEGK | | MASNRAPGV |
| | YIYYADSV | GASTRAT | PAPEFEGGPSVFLFPP | | TYYPDSVK | | PDRFSGSGS |
| | KGRFTISR | GIPARFS | KPKDTLMISRTPEVTC | | GRFTISRD | | GTDFTLKIS |
| | DSSKNTLY | GSGSGTE | VVVDVSDEDGEVKFNW | | NAKNSLYL | | RVEAEDVGV |
| | LQMNSLRA | FTLTISS | YVDGVEVHNAKTKPRE | | QMNSLRAE | | YYCMQALRA |
| | EDTAVYYC | LQSEDFA | EQYNSTYRVVSVLTVL | | DTAVYYCA | | PFSFGGGTK |
| | ARILKNGK | VYYCQQY | HQDWLNGKEYKCKVSN | | RDVGDWYF | | VEIK (SEQ |
| | YIYYFDYW | NNWPPWT | KALAAPIEKTISKAKG | | DLWGRGTL | | ID NO: 42) |
| | GQGTLVTV | FGQGTKV | QPREPQVYTLPPSRDE | | VTVSS | | |
| | SS (SEQ | EIK | LTKNQVSLTCLVKGFY | | (SEQ ID NO: | | |
| | ID NO: | (SEQ ID | PSDIAVEWESNGQPEN | | 78) | | |
| | 156) | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ | | | | |
| | | | ID NO: 545) | | | | |
| TA52 | EVQLLESG | QSVLTQP | ASTKGPSVFPLAPSSK | GGGGSGGG | EVQLVESG | GGGGSG | DIVMTQSPL |
| | GAFVQPGG | PSASGAP | STSGGTAALGCLVKDY | GSGGGGSG | GGLVQPGG | GGGSGG | SLPVTPGEP |
| | SLRLSCAA | GQRVTIS | FPEPVTVSWNSGALTS | GGGS (SEQ | SLRLSCAA | GGSGGG | ASISCRSSR |
| | SGFTFSDY | CSGSYSN | GVHTFPAVLQSSGLYS | ID NO: 4) | SRFTFSDY | GS (SEQ | SLVHGSGDN |
| | YMSWVRQA | IGNSYVS | LSSVVTVPSSSLGTQT | | HMSWVRQA | ID NO: 4) | YLHWYLQKP |
| | PGKGLEWV | WYQQLPG | YICNVNHKPSNTKVDK | | PGKGLELV | | GQSPQLLIY |
| | SVISNSGG | TAPKLLI | KVEPKSCDKTHTCPPC | | ASIDTEGK | | MASNRAPGV |
| | STYYTDSV | YLNSQRP | PAPELLGEESVFLFPP | | TYYPDSVK | | PDRFSGSGS |
| | KGRFTISR | SGVPDRF | KPKDTLMISRTPEVTC | | GRFTISRD | | GTDFTLKIS |
| | DNSKNTLY | SGSKSGT | VVVDVSHEDPEVKFNW | | NAKNSLYL | | RVEAEDVGV |
| | LQINSLRA | SASLAIS | YVDGVEVHNAKTKPRE | | QMNSLRAE | | YYCMQALRA |
| | EDTAVYYC | GLQSEDE | EQYNSTYRVVSVLTVL | | DTAVYYCA | | PFSFGGGTK |
| | TKDIGMTY | ADYYCAA | HQDWLNGKEYKCKVSN | | RDVGDWYF | | VEIK (SEQ |
| | FDYWGQGT | WDDLNVW | KALPAPIEKTISKAKG | | DLWGRGTL | | ID NO: 42) |
| | LVTVSS | VFGGGTK | QPREPQVYTLPPSRDE | | VTVSS | | |
| | (SEQ ID | LTVL | LTKNQVSLTCLVKGFY | | (SEQ ID NO: | | |
| | NO: 187) | (SEQ ID | PSDIAVEWESNGQPEN | | 78) | | |
| | | NO: | NYKTTPPVLDSDGSFF | | | | |
| | | 344) | LYSKLTVDKSRWQQGN | | | | |
| | | | VFSCSVMHEALHNHYT | | | | |
| | | | QKSLSLSPG (SEQ | | | | |
| | | | ID NO: 543) | | | | |
| TA53 | EVQLLESG | QSVLTQP | ASTKGPSVFPLAPSSK | GGGGSGGG | EVQLVESG | GGGGSG | DIVMTQSPL |
| | GAFVQPGG | PSASGAP | STSGGTAALGCLVKDY | GSGGGGSG | GGLVQPGG | GGGSGG | SLPVTPGEP |
| | SLRLSCAA | GQRVTIS | FPEPVTVSWNSGALTS | GGGS (SEQ | SLRLSCAA | GGSGGG | ASISCRSSR |
| | SGFTFSDY | CSGSYSN | GVHTFPAVLQSSGLYS | ID NO: 4) | SRFTFSDY | GS (SEQ | SLVHGSGDN |
| | YMSWVRQA | IGNSYVS | LSSVVTVPSSSLGTQT | | HMSWVRQA | ID NO: 4) | YLHWYLQKP |
| | PGKGLEWV | WYQQLPG | YICNVNHKPSNTKVDK | | PGKGLELV | | GQSPQLLIY |
| | SVISNSGG | TAPKLLI | KVEPKSCDKTHTCPPC | | ASIDTEGK | | MASNRAPGV |
| | STYYTDSV | YLNSQRP | PAPELLGDGSAFLFPP | | TYYPDSVK | | PDRFSGSGS |
| | KGRFTISR | SGVPDRF | KPKDTLMISRTPEVTC | | GRFTISRD | | GTDFTLKIS |
| | DNSKNTLY | SGSKSGT | VVVDVSHDEPEVKFNW | | NAKNSLYL | | RVEAEDVGV |
| | LQINSLRA | SASLAIS | YVDGVEVHNAKTKPRE | | QMNSLRAE | | YYCMQALRA |
| | EDTAVYYC | GLQSEDE | EQYNSTYRVVSVLTVL | | DTAVYYCA | | PFSFGGGTK |
| | TKDIGMTY | ADYYCAA | HQDWLNGKEYKCKVSN | | RDVGDWYF | | VEIK (SEQ |
| | FDYWGQGT | WDDLNVW | KALPRPIEKTISKAKG | | DLWGRGTL | | ID NO: 42) |
| | LVTVSS | VFGGGTK | QPREPQVYTLPPSRDE | | VTVSS | | |
| | (SEQ ID | LTVL | LTKNQVSLTCLVKGFY | | (SEQ ID NO: | | |
| | NO: 187) | (SEQ ID | PSDIAVEWESNGQPEN | | 78) | | |
| | | NO: | NYKTTPPVLDSDGSFF | | | | |
| | | 344) | LYSKLTVDKSRWQQGN | | | | |
| | | | VFSCSVMHEALHNHYT | | | | |
| | | | QKSLSLSPG (SEQ | | | | |
| | | | ID NO: 544) | | | | |
| TA54 | EVQLLESG | QSVLTQP | ASTKGPSVFPLAPSSK | GGGGSGGG | EVQLVESG | GGGGSG | DIVMTQSPL |
| | GAFVQPGG | PSASGAP | STSGGTAALGCLVKDY | GSGGGGSG | GGLVQPGG | GGGSGG | SLPVTPGEP |
| | SLRLSCAA | GQRVTIS | FPEPVTVSWNSGALTS | GGGS (SEQ | SLRLSCAA | GGSGGG | ASISCRSSR |
| | SGFTFSDY | CSGSYSN | GVHTFPAVLQSSGLYS | ID NO: 4) | SRFTFSDY | GS (SEQ | SLVHGSGDN |
| | YMSWVRQA | IGNSYVS | LSSVVTVPSSSLGTQT | | HMSWVRQA | ID NO: 4) | YLHWYLQKP |
| | PGKGLEWV | WYQQLPG | YICNVNHKPSNTKVDK | | PGKGLELV | | GQSPQLLIY |
| | SVISNSGG | TAPKLLI | KVEPKSCDKTHTCPPC | | ASIDTEGK | | MASNRAPGV |
| | STYYTDSV | YLNSQRP | PAPEFEGGPSVFLFPP | | TYYPDSVK | | PDRFSGSGS |
| | KGRFTISR | SGVPDRF | KPKDTLMISRTPEVTC | | GRFTISRD | | GTDFTLKIS |
| | DNSKNTLY | SGSKSGT | VVVDVSDEDGEVKFNW | | NAKNSLYL | | RVEAEDVGV |
| | LQINSLRA | SASLAIS | YVDGVEVHNAKTKPRE | | QMNSLRAE | | YYCMQALRA |
| | EDTAVYYC | GLQSEDE | EQYNSTYRVVSVLTVL | | DTAVYYCA | | PFSFGGGTK |
| | TKDIGMTY | ADYYCAA | HQDWLNGKEYKCKVSN | | RDVGDWYF | | VEIK (SEQ |
| | FDYWGQGT | WDDLNVW | KALAAPIEKTISKAKG | | DLWGRGTL | | ID NO: 42) |
| | LVTVSS | VFGGGTK | QPREPQVYTLPPSRDE | | VTVSS | | |
| | (SEQ ID | LTVL | LTKNQVSLTCLVKGFY | | (SEQ ID NO: | | |
| | NO: 187) | (SEQ ID | PSDIAVEWESNGQPEN | | 78) | | |
| | | NO: | NYKTTPPVLDSDGSFF | | | | |
| | | 344) | LYSKLTVDKSRWQQGN | | | | |
| | | | VFSCSVMHEALHNHYT | | | | |
| | | | QKSLSLSPG (SEQ | | | | |
| | | | ID NO: 545) | | | | |
| TA55 | QVTLKESG | QAVVTQE | ASTKGPSVFPLAPSSK | GGGGSGGG | EVQLVESG | GGGGSG | DIVMTQSPL |
| | PGILQPSQ | SALTTSP | STSGGTAALGCLVKDY | GSGGGGSG | GGLVQPGG | GGGSGG | SLPVTPGEP |
| | TLSLTCSF | GETVTLT | FPEPVTVSWNSGALTS | GGGS (SEQ | SLRLSCAA | GGSGGG | ASISCRSSR |
| | SGFSLSTF | CRSSTGA | GVHTFPAVLQSSGLYS | ID NO: 4) | SRFTFSDY | GS (SEQ | SLVHGSGDN |
| | GMGVGWIR | VTTSNYA | LSSVVTVPSSSLGTQT | | HMSWVRQA | ID NO: 4) | YLHWYLQKP |
| | QPSGKGLE | NWVQEKP | YICNVNHKPSNTKVDK | | PGKGLELV | | GQSPQLLIY |
| | WLAHIWWD | DHLFTGL | KVEPKSCDKTHTCPPC | | ASIDTEGK | | MASNRAPGV |
| | DDKYYNPA | IGGTNNR | PAPELLGEESVFLFPP | | TYYPDSVK | | PDRFSGSGS |
| | LKSRLTIS | APGVPAR | KPKDTLMISRTPEVTC | | GRFTISRD | | GTDFTLKIS |
| | KDTSKNQV | FSGSLIG | VVVDVSHEDPEVKFNW | | NAKNSLYL | | RVEAEDVGV |
| | FLKIANVD | DKAALTI | YVDGVEVHNAKTKPRE | | QMNSLRAE | | YYCMQALRA |
| | TADTATYY | TGAQTED | EQYNSTYRVVSVLTVL | | DTAVYYCA | | PFSFGGGTK |
| | CARIITTA | EAIYFCA | HQDWLNGKEYKCKVSN | | RDVGDWYF | | VEIK (SEQ |
| | WYFDVWGT | LWYSNHF | KALPAPIEKTISKAKG | | DLWGRGTL | | ID NO: 42) |
| | GTTVTVSS | IFGSGTK | QPREPQVYTLPPSRDE | | VTVSS | | |
| | (SEQ ID | VTVL | LTKNQVSLTCLVKGFY | | (SEQ ID NO: | | |
| | NO: 290) | (SEQ ID | PSDIAVEWESNGQPEN | | 78) | | |
| | | NO: | NYKTTPPVLDSDGSFF | | | | |
| | | 388) | LYSKLTVDKSRWQQGN | | | | |
| | | | VFSCSVMHEALHNHYT | | | | |
| | | | QKSLSLSPG (SEQ | | | | |
| | | | ID NO: 543) | | | | |
| TA56 | QVTLKESG | QAVVTQE | ASTKGPSVFPLAPSSK | GGGGSGGG | EVQLVESG | GGGGSG | DIVMTQSPL |
| | PGILQPSQ | SALTTSP | STSGGTAALGCLVKDY | GSGGGGSG | GGLVQPGG | GGGSGG | SLPVTPGEP |
| | TLSLTCSF | GETVTLT | FPEPVTVSWNSGALTS | GGGS (SEQ | SLRLSCAA | GGSGGG | ASISCRSSR |
| | SGFSLSTF | CRSSTGA | GVHTFPAVLQSSGLYS | ID NO: 4) | SRFTFSDY | GS (SEQ | SLVHGSGDN |
| | GMGVGWIR | VTTSNYA | LSSVVTVPSSSLGTQT | | HMSWVRQA | ID NO: 4) | YLHWYLQKP |
| | QPSGKGLE | NWVQEKP | YICNVNHKPSNTKVDK | | PGKGLELV | | GQSPQLLIY |
| | WLAHIWWD | DHLFTGL | KVEPKSCDKTHTCPPC | | ASIDTEGK | | MASNRAPGV |
| | DDKYYNPA | IGGTNNR | PAPELLGDGSAFLFPP | | TYYPDSVK | | PDRFSGSGS |
| | LKSRLTIS | APGVPAR | KPKDTLMISRTPEVTC | | GRFTISRD | | GTDFTLKIS |
| | KDTSKNQV | FSGSLIG | VVVDVSHDEPEVKFNW | | NAKNSLYL | | RVEAEDVGV |
| | FLKIANVD | DKAALTI | YVDGVEVHNAKTKPRE | | QMNSLRAE | | YYCMQALRA |
| | TADTATYY | TGAQTED | EQYNSTYRVVSVLTVL | | DTAVYYCA | | PFSFGGGTK |
| | CARIITTA | EAIYFCA | HQDWLNGKEYKCKVSN | | RDVGDWYF | | VEIK (SEQ |
| | WYFDVWGT | LWYSNHF | KALPRPIEKTISKAKG | | DLWGRGTL | | ID NO: 42) |
| | GTTVTVSS | IFGSGTK | QPREPQVYTLPPSRDE | | VTVSS | | |
| | (SEQ ID | VTVL | LTKNQVSLTCLVKGFY | | (SEQ ID NO: | | |
| | NO: 290) | (SEQ ID | PSDIAVEWESNGQPEN | | 78) | | |
| | | NO: | NYKTTPPVLDSDGSFF | | | | |
| | | 388) | LYSKLTVDKSRWQQGN | | | | |
| | | | VFSCSVMHEALHNHYT | | | | |
| | | | QKSLSLSPG (SEQ | | | | |
| | | | ID NO: 544) | | | | |
| TA57 | QVTLKESG | QAVVTQE | ASTKGPSVFPLAPSSK | GGGGSGGG | EVQLVESG | GGGGSG | DIVMTQSPL |
| | PGILQPSQ | SALTTSP | STSGGTAALGCLVKDY | GSGGGGSG | GGLVQPGG | GGGSGG | SLPVTPGEP |
| | TLSLTCSF | GETVTLT | FPEPVTVSWNSGALTS | GGGS (SEQ | SLRLSCAA | GGSGGG | ASISCRSSR |
| | SGFSLSTF | CRSSTGA | GVHTFPAVLQSSGLYS | ID NO: 4) | SRFTFSDY | GS (SEQ | SLVHGSGDN |
| | GMGVGWIR | VTTSNYA | LSSVVTVPSSSLGTQT | | HMSWVRQA | ID NO: 4) | YLHWYLQKP |
| | QPSGKGLE | NWVQEKP | YICNVNHKPSNTKVDK | | PGKGLELV | | GQSPQLLIY |
| | WLAHIWWD | DHLFTGL | KVEPKSCDKTHTCPPC | | ASIDTEGK | | MASNRAPGV |
| | DDKYYNPA | IGGTNNR | PAPEFEGGPSVFLFPP | | TYYPDSVK | | PDRFSGSGS |
| | LKSRLTIS | APGVPAR | KPKDTLMISRTPEVTC | | GRFTISRD | | GTDFTLKIS |
| | KDTSKNQV | FSGSLIG | VVVDVSDEDGEVKFNW | | NAKNSLYL | | RVEAEDVGV |
| | FLKIANVD | DKAALTI | YVDGVEVHNAKTKPRE | | QMNSLRAE | | YYCMQALRA |
| | TADTATYY | TGAQTED | EQYNSTYRVVSVLTVL | | DTAVYYCA | | PFSFGGGTK |
| | CARIITTA | EAIYFCA | HQDWLNGKEYKCKVSN | | RDVGDWYF | | VEIK (SEQ |
| | WYFDVWGT | LWYSNHF | KALAAPIEKTISKAKG | | DLWGRGTL | | ID NO: 42) |
| | GTTVTVSS | IFGSGTK | QPREPQVYTLPPSRDE | | VTVSS | | |
| | (SEQ ID | VTVL | LTKNQVSLTCLVKGFY | | (SEQ ID NO: | | |
| | NO: 290) | (SEQ ID | PSDIAVEWESNGQPEN | | 78) | | |
| | | NO: | NYKTTPPVLDSDGSFF | | | | |
| | | 388) | LYSKLTVDKSRWQQGN | | | | |
| | | | VFSCSVMHEALHNHYT | | | | |
| | | | QKSLSLSPG (SEQ | | | | |
| | | | ID NO: 545) | | | | |
| TA58 | EVQLLESG | EIVMTQS | ASTKGPSVFPLAPSSK | GGGGSGGG | EVQLVESG | GGGGSG | DIQMTQSPS |
| | GGLVQPGG | PATLSVS | STSGGTAALGCLVKDY | GSGGGGSG | GGLVQPGR | GGGSGG | SLSASVGDR |
| | SLRLSCAA | PGERATL | FPEPVTVSWNSGALTS | GGGS (SEQ | SLRLSCAA | GGSGGG | VTITCRASQ |
| | SGFTFSSD | SCRASQS | GVHTFPAVLQSSGLYS | ID NO: 4) | SGSTLSSY | GS (SEQ | GIGSYLAWY |
| | AMNWVRQA | VSSNLAW | LSSVVTVPSSSLGTQT | | GMHWVRQA | ID NO: 4) | QQKPGKAPK |
| | PGKGLEWV | YQQKPGQ | YICNVNHKPSNTKVDK | | PGKGLEWV | | LLIYEASRL |
| | STIYSGGS | APRLLIY | KVEPKSCDKTHTCPPC | | SAISYDAS | | ESGVPSRES |
| | TYYADSVK | GASTRAT | PAPELLGEESVFLFPP | | TIDYADSV | | GSGSGTDFT |
| | GRFTISRD | GIPARFS | KPKDTLMISRTPEVTC | | EGRFTISR | | LTISSLQPE |
| | NSKNTLYL | GSGSGTE | VVVDVSHEDPEVKFNW | | DNAKNSLY | | DVATYYCQQ |
| | QMNSLRAE | FTLTISS | YVDGVEVHNAKTKPRE | | LQMNSLRA | | GYNAPITFG |
| | DTAVYYCA | LQSEDFA | EQYNSTYRVVSVLTVL | | EDTAVYYC | | GGTKVEIK |
| | RGGNFYNY | VYYCQQY | HQDWLNGKEYKCKVSN | | ARDGISGI | | (SEQ ID NO: |
| | FDYWGQGT | NNWPPWT | KALPAPIEKTISKAKG | | DYWGQGTL | | 44) |
| | LVTVSS | FGQGTKV | QPREPQVYTLPPSRDE | | VTVSS | | |
| | (SEQ ID | EIK | LTKNQVSLTCLVKGFY | | (SEQ ID NO: | | |
| | NO: 136) | (SEQ ID | PSDIAVEWESNGQPEN | | 90) | | |
| | | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ | | | | |
| | | | ID NO: 543) | | | | |
| TA59 | EVQLLESG | EIVMTQS | ASTKGPSVFPLAPSSK | GGGGSGGG | EVQLVESG | GGGGSG | DIQMTQSPS |
| | GGLVQPGG | PATLSVS | STSGGTAALGCLVKDY | GSGGGGSG | GGLVQPGR | GGGSGG | SLSASVGDR |
| | SLRLSCAA | PGERATL | FPEPVTVSWNSGALTS | GGGS (SEQ | SLRLSCAA | GGSGGG | VTITCRASQ |
| | SGFTFSSD | SCRASQS | GVHTFPAVLQSSGLYS | ID NO: 4) | SGSTLSSY | GS (SEQ | GIGSYLAWY |
| | AMNWVRQA | VSSNLAW | LSSVVTVPSSSLGTQT | | GMHWVRQA | ID NO: 4) | QQKPGKAPK |
| | PGKGLEWV | YQQKPGQ | YICNVNHKPSNTKVDK | | PGKGLEWV | | LLIYEASRL |
| | STIYSGGS | APRLLIY | KVEPKSCDKTHTCPPC | | SAISYDAS | | ESGVPSRES |
| | TYYADSVK | GASTRAT | PAPELLGDGSAFLFPP | | TIDYADSV | | GSGSGTDFT |
| | GRFTISRD | GIPARFS | KPKDTLMISRTPEVTC | | EGRFTISR | | LTISSLQPE |
| | NSKNTLYL | GSGSGTE | VVVDVSHDEPEVKFNW | | DNAKNSLY | | DVATYYCQQ |
| | QMNSLRAE | FTLTISS | YVDGVEVHNAKTKPRE | | LQMNSLRA | | GYNAPITFG |
| | DTAVYYCA | LQSEDFA | EQYNSTYRVVSVLTVL | | EDTAVYYC | | GGTKVEIK |
| | RGGNFYNY | VYYCQQY | HQDWLNGKEYKCKVSN | | ARDGISGI | | (SEQ ID NO: |
| | FDYWGQGT | NNWPPWT | KALPRPIEKTISKAKG | | DYWGQGTL | | 44) |
| | LVTVSS | FGQGTKV | QPREPQVYTLPPSRDE | | VTVSS | | |
| | (SEQ ID | EIK | LTKNQVSLTCLVKGFY | | (SEQ ID NO: | | |
| | NO: 136) | (SEQ ID | PSDIAVEWESNGQPEN | | 90) | | |
| | | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ | | | | |
| | | | ID NO: 544) | | | | |
| TA60 | EVQLLESG | EIVMTQS | ASTKGPSVFPLAPSSK | GGGGSGGG | EVQLVESG | GGGGSG | DIQMTQSPS |
| | GGLVQPGG | PATLSVS | STSGGTAALGCLVKDY | GSGGGGSG | GGLVQPGR | GGGSGG | SLSASVGDR |
| | SLRLSCAA | PGERATL | FPEPVTVSWNSGALTS | GGGS (SEQ | SLRLSCAA | GGSGGG | VTITCRASQ |
| | SGFTFSSD | SCRASQS | GVHTFPAVLQSSGLYS | ID NO: 4) | SGSTLSSY | GS (SEQ | GIGSYLAWY |
| | AMNWVRQA | VSSNLAW | LSSVVTVPSSSLGTQT | | GMHWVRQA | ID NO: 4) | QQKPGKAPK |
| | PGKGLEWV | YQQKPGQ | YICNVNHKPSNTKVDK | | PGKGLEWV | | LLIYEASRL |
| | STIYSGGS | APRLLIY | KVEPKSCDKTHTCPPC | | SAISYDAS | | ESGVPSRES |
| | TYYADSVK | GASTRAT | PAPEFEGGPSVFLFPP | | TIDYADSV | | GSGSGTDFT |
| | GRFTISRD | GIPARFS | KPKDTLMISRTPEVTC | | EGRFTISR | | LTISSLQPE |
| | NSKNTLYL | GSGSGTE | VVVDVSDEDGEVKFNW | | DNAKNSLY | | DVATYYCQQ |
| | QMNSLRAE | FTLTISS | YVDGVEVHNAKTKPRE | | LQMNSLRA | | GYNAPITFG |
| | DTAVYYCA | LQSEDFA | EQYNSTYRVVSVLTVL | | EDTAVYYC | | GGTKVEIK |
| | RGGNFYNY | VYYCQQY | HQDWLNGKEYKCKVSN | | ARDGISGI | | (SEQ ID NO: |
| | FDYWGQGT | NNWPPWT | KALAAPIEKTISKAKG | | DYWGQGTL | | 44) |
| | LVTVSS | FGQGTKV | QPREPQVYTLPPSRDE | | VTVSS | | |
| | (SEQ ID | EIK | LTKNQVSLTCLVKGFY | | (SEQ ID NO: | | |
| | NO: 136) | (SEQ ID | PSDIAVEWESNGQPEN | | 90) | | |
| | | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ | | | | |
| | | | ID NO: 545) | | | | |
| TA61 | EVQLLESG | EIVMTQS | ASTKGPSVFPLAPSSK | GGGGSGGG | EVQLVESG | GGGGSG | DIQMTQSPS |
| | GGLVQPGG | PATLSVS | STSGGTAALGCLVKDY | GSGGGGSG | GGLVQPGR | GGGSGG | SLSASVGDR |
| | SLRLSCAA | PGERATL | FPEPVTVSWNSGALTS | GGGS (SEQ | SLRLSCAA | GGSGGG | VTITCRASQ |
| | SGFTFSDH | SCRASQS | GVHTFPAVLQSSGLYS | ID NO: 4) | SGSTLSSY | GS (SEQ | GIGSYLAWY |
| | YMSWVRQA | VSSNLAW | LSSVVTVPSSSLGTQT | | GMHWVRQA | ID NO: 4) | QQKPGKAPK |
| | PGKGLEWV | YQQKPGQ | YICNVNHKPSNTKVDK | | PGKGLEWV | | LLIYEASRL |
| | STISSGGN | APRLLIY | KVEPKSCDKTHTCPPC | | SAISYDAS | | ESGVPSRES |
| | YIYYADSV | GASTRAT | PAPELLGEESVFLFPP | | TIDYADSV | | GSGSGTDFT |
| | KGRFTISR | GIPARFS | KPKDTLMISRTPEVTC | | EGRFTISR | | LTISSLQPE |
| | DSSKNTLY | GSGSGTE | VVVDVSHEDPEVKFNW | | DNAKNSLY | | DVATYYCQQ |
| | LQMNSLRA | FTLTISS | YVDGVEVHNAKTKPRE | | LQMNSLRA | | GYNAPITFG |
| | EDTAVYYC | LQSEDFA | EQYNSTYRVVSVLIVL | | EDTAVYYC | | GGTKVEIK |
| | ARILKNGK | VYYCQQY | HQDWLNGKEYKCKVSN | | ARDGISGI | | (SEQ ID NO: |
| | YIYYFDYW | NNWPPWT | KALPAPIEKTISKAKG | | DYWGQGTL | | 44) |
| | GQGTLVTV | FGQGTKV | QPREPQVYTLPPSRDE | | VTVSS | | |
| | SS (SEQ | EIK | LTKNQVSLTCLVKGFY | | (SEQ ID NO: | | |
| | ID NO: | (SEQ ID | PSDIAVEWESNGQPEN | | 90) | | |
| | 156) | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ | | | | |
| | | | ID NO: 543) | | | | |
| TA62 | EVQLLESG | EIVMTQS | ASTKGPSVFPLAPSSK | GGGGSGGG | EVQLVESG | GGGGSG | DIQMTQSPS |
| | GGLVQPGG | PATLSVS | STSGGTAALGCLVKDY | GSGGGGSG | GGLVQPGR | GGGSGG | SLSASVGDR |
| | SLRLSCAA | PGERATL | FPEPVTVSWNSGALTS | GGGS (SEQ | SLRLSCAA | GGSGGG | VTITCRASQ |
| | SGFTFSDH | SCRASQS | GVHTFPAVLQSSGLYS | ID NO: 4) | SGSTLSSY | GS (SEQ | GIGSYLAWY |
| | YMSWVRQA | VSSNLAW | LSSVVTVPSSSLGTQT | | GMHWVRQA | ID NO: 4) | QQKPGKAPK |
| | PGKGLEWV | YQQKPGQ | YICNVNHKPSNTKVDK | | PGKGLEWV | | LLIYEASRL |
| | STISSGGN | APRLLIY | KVEPKSCDKTHTCPPC | | SAISYDAS | | ESGVPSRES |
| | YIYYADSV | GASTRAT | PAPELLGDGSAFLFPP | | TIDYADSV | | GSGSGTDFT |
| | KGRFTISR | GIPARFS | KPKDTLMISRTPEVTC | | EGRFTISR | | LTISSLQPE |
| | DSSKNTLY | GSGSGTE | VVVDVSHDEPEVKFNW | | DNAKNSLY | | DVATYYCQQ |
| | LQMNSLRA | FTLTISS | YVDGVEVHNAKTKPRE | | LQMNSLRA | | GYNAPITFG |
| | EDTAVYYC | LQSEDFA | EQYNSTYRVVSVLTVL | | EDTAVYYC | | GGTKVEIK |
| | ARILKNGK | VYYCQQY | HQDWLNGKEYKCKVSN | | ARDGISGI | | (SEQ ID NO: |
| | YIYYFDYW | NNWPPWT | KALPRPIEKTISKAKG | | DYWGQGTL | | 44) |
| | GQGTLVTV | FGQGTKV | QPREPQVYTLPPSRDE | | VTVSS | | |
| | SS (SEQ | EIK | LTKNQVSLTCLVKGFY | | (SEQ ID NO: | | |
| | ID NO: | (SEQ ID | PSDIAVEWESNGQPEN | | 90) | | |
| | 156) | NO: | NYKTTPPVLDSDGSFF | | | | |
| | | 323) | LYSKLTVDKSRWQQGN | | | | |
| | | | VFSCSVMHEALHNHYT | | | | |
| | | | QKSLSLSPG (SEQ | | | | |
| | | | ID NO: 544) | | | | |
| TA63 | EVQLLESG | EIVMTQS | ASTKGPSVFPLAPSSK | GGGGSGGG | EVQLVESG | GGGGSG | DIQMTQSPS |
| | GGLVQPGG | PATLSVS | STSGGTAALGCLVKDY | GSGGGGSG | GGLVQPGR | GGGSGG | SLSASVGDR |
| | SLRLSCAA | PGERATL | FPEPVTVSWNSGALTS | GGGS (SEQ | SLRLSCAA | GGSGGG | VTITCRASQ |
| | SGFTFSDH | SCRASQS | GVHTFPAVLQSSGLYS | ID NO: 4) | SGSTLSSY | GS (SEQ | GIGSYLAWY |
| | YMSWVRQA | VSSNLAW | LSSVVTVPSSSLGTQT | | GMHWVRQA | ID NO: 4) | QQKPGKAPK |
| | PGKGLEWV | YQQKPGQ | YICNVNHKPSNTKVDK | | PGKGLEWV | | LLIYEASRL |
| | STISSGGN | APRLLIY | KVEPKSCDKTHTCPPC | | SAISYDAS | | ESGVPSRES |
| | YIYYADSV | GASTRAT | PAPEFEGGPSVFLFPP | | TIDYADSV | | GSGSGTDFT |
| | KGRFTISR | GIPARFS | KPKDTLMISRTPEVTC | | EGRFTISR | | LTISSLQPE |
| | DSSKNTLY | GSGSGTE | VVVDVSDEDGEVKFNW | | DNAKNSLY | | DVATYYCQQ |
| | LQMNSLRA | FTLTISS | YVDGVEVHNAKTKPRE | | LQMNSLRA | | GYNAPITFG |
| | EDTAVYYC | LQSEDFA | EQYNSTYRVVSVLTVL | | EDTAVYYC | | GGTKVEIK |
| | ARILKNGK | VYYCQQY | HQDWLNGKEYKCKVSN | | ARDGISGI | | (SEQ ID NO: |
| | YIYYFDYW | NNWPPWT | KALAAPIEKTISKAKG | | DYWGQGTL | | 44) |
| | GQGTLVTV | FGQGTKV | QPREPQVYTLPPSRDE | | VTVSS | | |
| | SS (SEQ | EIK | LTKNQVSLTCLVKGFY | | (SEQ ID NO: | | |
| | ID NO: | | PSDIAVEWESNGQPEN | | 90) | | |
| | 156) | | NYKTTPPVLDSDGSFF | | | | |
| | | | LYSKLTVDKSRWQQGN | | | | |
| | | | VFSCSVMHEALHNHYT | | | | |
| | | | QKSLSLSPG (SEQ | | | | |
| | | | ID NO: 545) | | | | |
| TA64 | EVQLLESG | QSVLTQP | ASTKGPSVFPLAPSSK | GGGGSGGG | EVQLVESG | GGGGSG | DIQMTQSPS |
| | GAFVQPGG | PSASGAP | STSGGTAALGCLVKDY | GSGGGGSG | GGLVQPGR | GGGSGG | SLSASVGDR |
| | SLRLSCAA | GQRVTIS | FPEPVTVSWNSGALTS | GGGS (SEQ | SLRLSCAA | GGSGGG | VTITCRASQ |
| | SGFTFSDY | CSGSYSN | GVHTFPAVLQSSGLYS | ID NO: 4) | SGSTLSSY | GS (SEQ | GIGSYLAWY |
| | YMSWVRQA | IGNSYVS | LSSVVTVPSSSLGTQT | | GMHWVRQA | ID NO: 4) | QQKPGKAPK |
| | PGKGLEWV | WYQQLPG | YICNVNHKPSNTKVDK | | PGKGLEWV | | LLIYEASRL |
| | SVISNSGG | TAPKLLI | KVEPKSCDKTHTCPPC | | SAISYDAS | | ESGVPSRES |
| | STYYTDSV | YLNSQRP | PAPELLGEESVFLFPP | | TIDYADSV | | GSGSGTDFT |
| | KGRFTISR | SGVPDRF | KPKDTLMISRTPEVTC | | EGRFTISR | | LTISSLQPE |
| | DNSKNTLY | SGSKSGT | VVVDVSHEDPEVKFNW | | DNAKNSLY | | DVATYYCQQ |
| | LQINSLRA | SASLAIS | YVDGVEVHNAKTKPRE | | LQMNSLRA | | GYNAPITFG |
| | EDTAVYYC | GLQSEDE | EQYNSTYRVVSVLTVL | | EDTAVYYC | | GGTKVEIK |
| | TKDIGMTY | ADYYCAA | HQDWLNGKEYKCKVSN | | ARDGISGI | | (SEQ ID NO: |
| | FDYWGQGT | WDDLNVW | KALPAPIEKTISKAKG | | DYWGQGTL | | 44) |
| | LVTVSS | VFGGGTK | QPREPQVYTLPPSRDE | | VTVSS | | |
| | (SEQ ID | LTVL | LTKNQVSLTCLVKGFY | | (SEQ ID NO: | | |
| | NO: 187) | (SEQ ID | PSDIAVEWESNGQPEN | | 90) | | |
| | | NO: | NYKTTPPVLDSDGSFF | | | | |
| | | 344) | LYSKLTVDKSRWQQGN | | | | |
| | | | VFSCSVMHEALHNHYT | | | | |
| | | | QKSLSLSPG (SEQ | | | | |
| | | | ID NO: 543) | | | | |
| TA65 | EVQLLESG | QSVLTQP | ASTKGPSVFPLAPSSK | GGGGSGGG | EVQLVESG | GGGGSG | DIQMTQSPS |
| | GAFVQPGG | PSASGAP | STSGGTAALGCLVKDY | GSGGGGSG | GGLVQPGR | GGGSGG | SLSASVGDR |
| | SLRLSCAA | GQRVTIS | FPEPVTVSWNSGALTS | GGGS (SEQ | SLRLSCAA | GGSGGG | VTITCRASQ |
| | SGFTFSDY | CSGSYSN | GVHTFPAVLQSSGLYS | ID NO: 4) | SGSTLSSY | GS (SEQ | GIGSYLAWY |
| | YMSWVRQA | IGNSYVS | LSSVVTVPSSSLGTQT | | GMHWVRQA | ID NO: 4) | QQKPGKAPK |
| | PGKGLEWV | WYQQLPG | YICNVNHKPSNTKVDK | | PGKGLEWV | | LLIYEASRL |
| | SVISNSGG | TAPKLLI | KVEPKSCDKTHTCPPC | | SAISYDAS | | ESGVPSRES |
| | STYYTDSV | YLNSQRP | PAPELLGDGSAFLFPP | | TIDYADSV | | GSGSGTDFT |
| | KGRFTISR | SGVPDRF | KPKDTLMISRTPEVTC | | EGRFTISR | | LTISSLQPE |
| | DNSKNTLY | SGSKSGT | VVVDVSHDEPEVKFNW | | DNAKNSLY | | DVATYYCQQ |
| | LQINSLRA | SASLAIS | YVDGVEVHNAKTKPRE | | LQMNSLRA | | GYNAPITFG |
| | EDTAVYYC | GLQSEDE | EQYNSTYRVVSVLTVL | | EDTAVYYC | | GGTKVEIK |
| | TKDIGMTY | ADYYCAA | HQDWLNGKEYKCKVSN | | ARDGISGI | | (SEQ ID NO: |
| | FDYWGQGT | WDDLNVW | KALPRPIEKTISKAKG | | DYWGQGTL | | 44) |
| | LVTVSS | VFGGGTK | QPREPQVYTLPPSRDE | | VTVSS | | |
| | (SEQ ID | LTVL | LTKNQVSLTCLVKGFY | | (SEQ ID NO: | | |
| | NO: 187) | (SEQ ID | PSDIAVEWESNGQPEN | | 90) | | |
| | | NO: | NYKTTPPVLDSDGSFF | | | | |
| | | 344) | LYSKLTVDKSRWQQGN | | | | |
| | | | VFSCSVMHEALHNHYT | | | | |
| | | | QKSLSLSPG (SEQ | | | | |
| | | | ID NO: 544) | | | | |
| TA66 | EVQLLESG | QSVLTQP | ASTKGPSVFPLAPSSK | GGGGSGGG | EVQLVESG | GGGGSG | DIQMTQSPS |
| | GAFVQPGG | PSASGAP | STSGGTAALGCLVKDY | GSGGGGSG | GGLVQPGR | GGGSGG | SLSASVGDR |
| | SLRLSCAA | GQRVTIS | FPEPVTVSWNSGALTS | GGGS (SEQ | SLRLSCAA | GGSGGG | VTITCRASQ |
| | SGFTFSDY | CSGSYSN | GVHTFPAVLQSSGLYS | ID NO: 4) | SGSTLSSY | GS (SEQ | GIGSYLAWY |
| | YMSWVRQA | IGNSYVS | LSSVVTVPSSSLGTQT | | GMHWVRQA | ID NO: 4) | QQKPGKAPK |
| | PGKGLEWV | WYQQLPG | YICNVNHKPSNTKVDK | | PGKGLEWV | | LLIYEASRL |
| | SVISNSGG | TAPKLLI | KVEPKSCDKTHTCPPC | | SAISYDAS | | ESGVPSRES |
| | STYYTDSV | YLNSQRP | PAPEFEGGPSVFLFPP | | TIDYADSV | | GSGSGTDFT |
| | KGRFTISR | SGVPDRF | KPKDTLMISRTPEVTC | | EGRFTISR | | LTISSLQPE |
| | DNSKNTLY | SGSKSGT | VVVDVSDEDGEVKFNW | | DNAKNSLY | | DVATYYCQQ |
| | LQINSLRA | SASLAIS | YVDGVEVHNAKTKPRE | | LQMNSLRA | | GYNAPITFG |
| | EDTAVYYC | GLQSEDE | EQYNSTYRVVSVLTVL | | EDTAVYYC | | GGTKVEIK |
| | TKDIGMTY | ADYYCAA | HQDWLNGKEYKCKVSN | | ARDGISGI | | (SEQ ID NO: |
| | FDYWGQGT | WDDLNVW | KALAAPIEKTISKAKG | | DYWGQGTL | | 44) |
| | LVTVSS | VFGGGTK | QPREPQVYTLPPSRDE | | VTVSS | | |
| | (SEQ ID | LTVL | LTKNQVSLTCLVKGFY | | (SEQ ID NO: | | |
| | NO: 187) | (SEQ ID | PSDIAVEWESNGQPEN | | 90) | | |
| | | NO: | NYKTTPPVLDSDGSFF | | | | |
| | | 344) | LYSKLTVDKSRWQQGN | | | | |
| | | | VFSCSVMHEALHNHYT | | | | |
| | | | QKSLSLSPG (SEQ | | | | |
| | | | ID NO: 545) | | | | |
| TA67 | QVTLKESG | QAVVTQE | ASTKGPSVFPLAPSSK | GGGGSGGG | EVQLVESG | GGGGSG | DIQMTQSPS |
| | PGILQPSQ | SALTTSP | STSGGTAALGCLVKDY | GSGGGGSG | GGLVQPGR | GGGSGG | SLSASVGDR |
| | TLSLTCSF | GETVTLT | FPEPVTVSWNSGALTS | GGGS (SEQ | SLRLSCAA | GGSGGG | VTITCRASQ |
| | SGFSLSTF | CRSSTGA | GVHTFPAVLQSSGLYS | ID NO: 4) | SGSTLSSY | GS (SEQ | GIGSYLAWY |
| | GMGVGWIR | VTTSNYA | LSSVVTVPSSSLGTQT | | GMHWVRQA | ID NO: 4) | QQKPGKAPK |
| | QPSGKGLE | NWVQEKP | YICNVNHKPSNTKVDK | | PGKGLEWV | | LLIYEASRL |
| | WLAHIWWD | DHLFTGL | KVEPKSCDKTHTCPPC | | SAISYDAS | | ESGVPSRES |
| | DDKYYNPA | IGGTNNR | PAPELLGEESVFLFPP | | TIDYADSV | | GSGSGTDFT |
| | LKSRLTIS | APGVPAR | KPKDTLMISRTPEVTC | | EGRFTISR | | LTISSLQPE |
| | KDTSKNQV | FSGSLIG | VVVDVSHEDPEVKFNW | | DNAKNSLY | | DVATYYCQQ |
| | FLKIANVD | DKAALTI | YVDGVEVHNAKTKPRE | | LQMNSLRA | | GYNAPITFG |
| | TADTATYY | TGAQTED | EQYNSTYRVVSVLTVL | | EDTAVYYC | | GGTKVEIK |
| | CARIITTA | EAIYFCA | HQDWLNGKEYKCKVSN | | ARDGISGI | | (SEQ ID NO: |
| | WYFDVWGT | LWYSNHF | KALPAPIEKTISKAKG | | DYWGQGTL | | 44) |
| | GTTVTVSS | IFGSGTK | QPREPQVYTLPPSRDE | | VTVSS | | |
| | (SEQ ID | VTVL | LTKNQVSLTCLVKGFY | | (SEQ ID NO: | | |
| | NO: 290) | (SEQ ID | PSDIAVEWESNGQPEN | | 90) | | |
| | | NO: | NYKTTPPVLDSDGSFF | | | | |
| | | 388) | LYSKLTVDKSRWQQGN | | | | |
| | | | VFSCSVMHEALHNHYT | | | | |
| | | | QKSLSLSPG (SEQ | | | | |
| | | | ID NO: 543) | | | | |
| TA68 | QVTLKESG | QAVVTQE | ASTKGPSVFPLAPSSK | GGGGSGGG | EVQLVESG | GGGGSG | DIQMTQSPS |
| | PGILQPSQ | SALTTSP | STSGGTAALGCLVKDY | GSGGGGSG | GGLVQPGR | GGGSGG | SLSASVGDR |
| | TLSLTCSF | GETVTLT | FPEPVTVSWNSGALTS | GGGS (SEQ | SLRLSCAA | GGSGGG | VTITCRASQ |
| | SGFSLSTF | CRSSTGA | GVHTFPAVLQSSGLYS | ID NO: 4) | SGSTLSSY | GS (SEQ | GIGSYLAWY |
| | GMGVGWIR | VTTSNYA | LSSVVTVPSSSLGTQT | | GMHWVRQA | ID NO: 4) | QQKPGKAPK |
| | QPSGKGLE | NWVQEKP | YICNVNHKPSNTKVDK | | PGKGLEWV | | LLIYEASRL |
| | WLAHIWWD | DHLFTGL | KVEPKSCDKTHTCPPC | | SAISYDAS | | ESGVPSRES |
| | DDKYYNPA | IGGTNNR | PAPELLGDGSAFLFPP | | TIDYADSV | | GSGSGTDFT |
| | LKSRLTIS | APGVPAR | KPKDTLMISRTPEVTC | | EGRFTISR | | LTISSLQPE |
| | KDTSKNQV | FSGSLIG | VVVDVSHDEPEVKFNW | | DNAKNSLY | | DVATYYCQQ |
| | FLKIANVD | DKAALTI | YVDGVEVHNAKTKPRE | | LQMNSLRA | | GYNAPITFG |
| | TADTATYY | TGAQTED | EQYNSTYRVVSVLTVL | | EDTAVYYC | | GGTKVEIK |
| | CARIITTA | EAIYFCA | HQDWLNGKEYKCKVSN | | ARDGISGI | | (SEQ ID NO: |
| | WYFDVWGT | LWYSNHF | KALPRPIEKTISKAKG | | DYWGQGTL | | 44) |
| | GTTVTVSS | IFGSGTK | QPREPQVYTLPPSRDE | | VTVSS | | |
| | (SEQ ID | VTVL | LTKNQVSLTCLVKGFY | | (SEQ ID NO: | | |
| | NO: 290) | (SEQ ID | PSDIAVEWESNGQPEN | | 90) | | |
| | | NO: | NYKTTPPVLDSDGSFF | | | | |
| | | 388) | LYSKLTVDKSRWQQGN | | | | |
| | | | VFSCSVMHEALHNHYT | | | | |
| | | | QKSLSLSPG (SEQ | | | | |
| | | | ID NO: 544) | | | | |
| TA69 | QVTLKESG | QAVVTQE | ASTKGPSVFPLAPSSK | GGGGSGGG | EVQLVESG | GGGGSG | DIQMTQSPS |
| | PGILQPSQ | SALTTSP | STSGGTAALGCLVKDY | GSGGGGSG | GGLVQPGR | GGGSGG | SLSASVGDR |
| | TLSLTCSF | GETVTLT | FPEPVTVSWNSGALTS | GGGS (SEQ | SLRLSCAA | GGSGGG | VTITCRASQ |
| | SGFSLSTF | CRSSTGA | GVHTFPAVLQSSGLYS | ID NO: 4) | SGSTLSSY | GS (SEQ | GIGSYLAWY |
| | GMGVGWIR | VTTSNYA | LSSVVTVPSSSLGTQT | | GMHWVRQA | ID NO: 4) | QQKPGKAPK |
| | QPSGKGLE | NWVQEKP | YICNVNHKPSNTKVDK | | PGKGLEWV | | LLIYEASRL |
| | WLAHIWWD | DHLFTGL | KVEPKSCDKTHTCPPC | | SAISYDAS | | ESGVPSRES |
| | DDKYYNPA | IGGTNNR | PAPEFEGGPSVFLFPP | | TIDYADSV | | GSGSGTDFT |
| | LKSRLTIS | APGVPAR | KPKDTLMISRTPEVTC | | EGRFTISR | | LTISSLQPE |
| | KDTSKNQV | FSGSLIG | VVVDVSDEDGEVKFNW | | DNAKNSLY | | DVATYYCQQ |
| | FLKIANVD | DKAALTI | YVDGVEVHNAKTKPRE | | LQMNSLRA | | GYNAPITFG |
| | TADTATYY | TGAQTED | EQYNSTYRVVSVLTVL | | EDTAVYYC | | GGTKVEIK |
| | CARIITTA | EAIYFCA | HQDWLNGKEYKCKVSN | | ARDGISGI | | (SEQ ID NO: |
| | WYFDVWGT | LWYSNHF | KALAAPIEKTISKAKG | | DYWGQGTL | | 44) |
| | GTTVTVSS | IFGSGTK | QPREPQVYTLPPSRDE | | VTVSS | | |
| | (SEQ ID | VTVL | LTKNQVSLTCLVKGFY | | (SEQ ID NO: | | |
| | NO: 290) | (SEQ ID | PSDIAVEWESNGQPEN | | 90) | | |
| | | NO: | NYKTTPPVLDSDGSFF | | | | |
| | | 388) | LYSKLTVDKSRWQQGN | | | | |
| | | | VFSCSVMHEALHNHYT | | | | |
| | | | QKSLSLSPG (SEQ | | | | |
| | | | ID NO: 545) | | | | |
| TA70 | QVTLKESG | QAVVTQE | ASTKGPSVFPLAPSSK | GGGGSGGG | EVQLVESG | GGGGSG | DIVMTQSPL |
| | PGILQPSQ | SALTTSP | STSGGTAALGCLVKDY | GSGGGGSG | GGLVQPGG | GGGSGG | SLPVTPGEP |
| | TLSLTCSF | GETVTLT | FPEPVTVSWNSGALTS | GGGS (SEQ | SLRLSCAA | GGSGGG | ASISCRSSR |
| | SGFSLSTF | CRSSTGA | GVHTFPAVLQSSGLYS | ID NO: 4) | SRFTFSDY | GS (SEQ | SLVHGSGDN |
| | GMGVGWIR | VTTSNYA | LSSVVTVPSSSLGTQT | | HMSWVRQA | ID NO: 4) | YLHWYLQKP |
| | QPSGKGLE | NWVQEKP | YICNVNHKPSNTKVDK | | PGKGLELV | | GQSPQLLIY |
| | WLAHIWWD | DHLFTGL | KVEPKSCDKTHTCPPC | | ASIDTEGK | | MASNRAPGV |
| | DDKYYNPA | IGGTNNR | PAPEAAGAPSVFLFPP | | TYYPDSVK | | PDRFSGSGS |
| | LKSRLTIS | APGVPAR | KPKDTLMISRTPEVTC | | GRFTISRD | | GTDFTLKIS |
| | KDTSKNQV | FSGSLIG | VVVDVSHEDPEVKFNW | | NAKNSLYL | | RVEAEDVGV |
| | FLKIANVD | DKAALTI | YVDGVEVHNAKTKPRE | | QMNSLRAE | | YYCMQALRA |
| | TADTATYY | TGAQTED | EQYNSTYRVVSVLTVL | | DTAVYYCA | | PFSFGGGTK |
| | CARIITTA | EAIYFCA | HQDWLNGKEYKCKVSN | | RDVGDWYF | | VEIK (SEQ |
| | WYFDVWGT | LWYSNHF | KALPAPIEKTISKAKG | | DLWGRGTL | | ID NO: 42) |
| | GTTVTVSS | IFGSGTK | QPREPQVYTLPPSRDE | | VTVSS | | |
| | (SEQ ID | VTVL | LTKNQVSLTCLVKGFY | | (SEQ ID NO: | | |
| | NO: 290) | (SEQ ID | PSDIAVEWESNGQPEN | | 78) | | |
| | | NO: | NYKTTPPVLDSDGSFF | | | | |
| | | 388) | LYSKLTVDKSRWQQGN | | | | |
| | | | VFSCSVMHEALHNHYT | | | | |
| | | | QKSLSLSPG (SEQ ID | | | | |
| | | | NO: 513) | | | | |
| TA71 | QVTLKESG | QAVVTQE | ASTKGPSVFPLAPSSK | GGGGSGGG | EVQLVESG | GGGGSG | DIVMTQSPL |
| | PGILQPSQ | SALTTSP | STSGGTAALGCLVKDY | GSGGGGSG | GGLVQPGG | GGGSGG | SLPVTPGEP |
| | TLSLTCSF | GETVTLT | FPEPVTVSWNSGALTS | GGGS (SEQ | SLRLSCAA | GGSGGG | ASISCRSSR |
| | SGFSLSTF | CRSSTGA | GVHTFPAVLQSSGLYS | ID NO: 4) | SRFTFSDY | GS (SEQ | SLVHGSGDN |
| | GMGVGWIR | VTTSNYA | LSSVVTVPSSSLGTQT | | HMSWVRQA | ID NO: 4) | YLHWYLQKP |
| | QPSGKGLE | NWVQEKP | YICNVNHKPSNTKVDK | | PGKGLELV | | GQSPQLLIY |
| | WLAHIWWD | DHLFTGL | KVEPKSCDKTHTCPPC | | ASIDTEGK | | MASNRAPGV |
| | DDKYYNPA | IGGTNNR | PAPEAAGAPSVFLFPP | | TYYPDSVK | | PDRFSGSGS |
| | LKSRLTIS | APGVPAR | KPKDTLMISRTPEVTC | | GRFTISRD | | GTDFTLKIS |
| | KDTSKNQV | FSGSLIG | VVVDVSHEDPEVKFNW | | NAKNSLYL | | RVEAEDVGV |
| | FLKIANVD | DKAALTI | YVDGVEVHNAKTKPRE | | QMNSLRAE | | YYCMQALRA |
| | TADTATYY | TGAQTED | EQYNSTYRVVSVLTVL | | DTAVYYCA | | PFSFGGGTK |
| | CARIITTA | EAIYFCA | HQDWLNGKEYKCKVSN | | RDVGDWYF | | VEIK (SEQ |
| | WYFDVWGT | LWYSNHF | KALPAPIEKTISKAKG | | DLWGRGTL | | ID NO: 42) |
| | GTTVTVSS | IFGSGTK | QPREPQVYTLPPSRDE | | VTVSS | | |
| | (SEQ ID | VTVL | LTKNQVSLTCLVKGFY | | (SEQ ID NO: | | |
| | NO: 290) | (SEQ ID | PSDIAVEWESNGQPEN | | 78) | | |
| | | NO: | NYKTTPPVLDSDGSFF | | | | |
| | | 388) | LYSKLTVDKSRWQQGN | | | | |
| | | | VFSCSVMHEALHNHYT | | | | |
| | | | QKSLSLSPG (SEQ ID | | | | |
| | | | NO: 513) | | | | |

In some embodiments, non-limiting example of molecules comprising an anti-PD-1 antibody, or an antigen-binding fragment thereof, include those set forth in Table 10 below. In some embodiments, the signal peptide is optional, and thus, non-limiting examples of molecule optionally comprising the signal peptide may comprise the signal peptide. In some embodiments, non-limiting examples of molecules optionally comprising the signal peptide may not comprise the signal peptide. In some embodiments, the molecule comprising an anti-PD-1 antibody, or an antigen-binding fragment thereof, comprises a Ck or a Cl amino acid sequence. In some embodiments, the Ck or Cl amino acid sequence is selected from any one of SEQ ID NO: 415, or SEQ ID NO: 416.

| | VH | | | VL | | |
|---|---|---|---|---|---|---|
| | Signal | | | Signal | | |
| ID | Peptide | VH | Fc | Peptide | VL | Ck/Cl |
| TA72 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | TTYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSENTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 130) | | | | ID NO: |
| | | | | | | 415) |
| TA73 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDHYMSW | QTYICNVNHKPSNTKVDKKVEPKSCD | (SEQ ID | SVSSNLAWY | VVCLLNNF |
| | NO: | VRQAPGRGL | KTHTCPPCPAPDLLGDDSVFLFPPKP | NO: | QQKPGQAPR | YPREAKVQ |
| | 588) | EWVSTISSG | KDTLMISRTPEVTCVVVDVSDEDGEV | 588) | LLIYGASTR | WKVDNALQ |
| | | GSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCAKI | AVEWESNGQPENNYKTTPPVLDSDGS | | YNNWPPWTF | KHKVYACE |
| | | LKNGNYIYY | FFLYSKLTVDKSRWQQGNVFSCSVMH | | GQGTKVEIK | VTHQGLSS |
| | | FDYWGQGTL | EALHNHYTQKSLSLSPG (SEQ ID | | (SEQ ID | PVTKSFNR |
| | | VTVSS | NO: 587) | | NO: 323) | GEC (SEQ |
| | | (SEQ ID | | | | ID NO: |
| | | NO: 131) | | | | 415) |
| TA74 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFTGYDMTW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAISWN | KDTLMISRTPEVTCVVVDVSDEDGEV | 588) | LLIYGASTR | WKVDNALQ |
| | | TGSTTYYAD | KFNWYVDGVEVHNAKTKPREEQYNST | | ATGIPARFS | SGNSQESV |
| | | SVKGRFTIS | YRVVSVLTVLHQDWLNGKEYKCKVSN | | GSGSGTEFT | TEQDSKDS |
| | | RDNSKNTLY | KALPRPIEKTISKAKGQPREPQVYTL | | LTISSLQSE | TYSLSSTL |
| | | LQMNSLRAE | PPSRDELTKNQVSLTCLVKGFYPSDI | | DFAVYYCQQ | TLSKADYE |
| | | DTAVYYCTR | AVEWESNGQPENNYKTTPPVLDSDGS | | YNNWPPWTF | KHKVYACE |
| | | GYDRKNYFE | FFLYSKLTVDKSRWQQGNVFSCSVMH | | GQGTKVEIK | VTHQGLSS |
| | | YWGQGTLVT | EALHNHYTQKSLSLSPG (SEQ ID | | (SEQ ID | PVTKSFNR |
| | | VSS (SEQ | NO: 587) | | NO: 323) | GEC (SEQ |
| | | ID NO: | | | | ID NO: |
| | | 132) | | | | 415) |
| TA75 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDHYMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTISSG | KDTLMISRTPEVTCVVVDVSDEDGEV | 588) | LLIYGASTR | WKVDNALQ |
| | | GSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCAKI | AVEWESNGQPENNYKTTPPVLDSDGS | | YNNWPPWTF | KHKVYACE |
| | | LKNGNYIYY | FFLYSKLTVDKSRWQQGNVFSCSVMH | | GQGTKVEIK | VTHQGLSS |
| | | FDYWGQGTL | EALHNHYTQKSLSLSPG (SEQ ID | | (SEQ ID | PVTKSFNR |
| | | VTVSS | NO: 587) | | NO: 323) | GEC (SEQ |
| | | (SEQ ID | | | | ID NO: |
| | | NO: 133) | | | | 415) |
| TA76 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCVASGF | FPAVLQSSGLYSLSSVVTVPSSSLGT | TGVHS | ATLSCRASQ | QLKSGTAS |
| | (SEQ ID | TFDDYGMTW | QTYICNVNHKPSNTKVDKKVEPKSCD | (SEQ ID | SVSSNLAWY | VVCLLNNF |
| | NO: | VRQASGKGL | KTHTCPPCPAPDLLGDDSVFLFPPKP | NO: | QQKPGQAPR | YPREAKVQ |
| | 588) | EFVATVNWN | KDTLMISRTPEVTCVVVDVSDEDGEV | 588) | LLIYGASTR | WKVDNALQ |
| | | GNKTYYADS | KFNWYVDGVEVHNAKTKPREEQYNST | | ATGIPARFS | SGNSQESV |
| | | MKGRFTISR | YRVVSVLTVLHQDWLNGKEYKCKVSN | | GSGSGTEFT | TEQDSKDS |
| | | NNSENTVYL | KALPRPIEKTISKAKGQPREPQVYTL | | LTISSLQSE | TYSLSSTL |
| | | EVNSLRDED | PPSRDELTKNQVSLTCLVKGFYPSDI | | DFAVYYCQQ | TLSKADYE |
| | | TAIYYCVKN | AVEWESNGQPENNYKTTPPVLDSDGS | | YNNWPPWTF | KHKVYACE |
| | | HEWKFEYWG | FFLYSKLTVDKSRWQQGNVFSCSVMH | | GQGTKVEIK | VTHQGLSS |
| | | QGTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 134) | | | | ID NO: |
| | | | | | | 415) |
| TA77 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | PSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 135) | | | | ID NO: |
| | | | | | | 415) |
| TA78 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSSDAMNW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTIYSG | KDTLMISRTPEVTCVVVDVSDEDGEV | 588) | LLIYGASTR | WKVDNALQ |
| | | GSTYYADSV | KFNWYVDGVEVHNAKTKPREEQYNST | | ATGIPARFS | SGNSQESV |
| | | KGRFTISRD | YRVVSVLTVLHQDWLNGKEYKCKVSN | | GSGSGTEFT | TEQDSKDS |
| | | NSKNTLYLQ | KALPRPIEKTISKAKGQPREPQVYTL | | LTISSLQSE | TYSLSSTL |
| | | MNSLRAEDT | PPSRDELTKNQVSLTCLVKGFYPSDI | | DFAVYYCQQ | TLSKADYE |
| | | AVYYCARGG | AVEWESNGQPENNYKTTPPVLDSDGS | | YNNWPPWTF | KHKVYACE |
| | | NFYNYFDYW | FFLYSKLTVDKSRWQQGNVFSCSVMH | | GQGTKVEIK | VTHQGLSS |
| | | GQGTLVTVS | EALHNHYTQKSLSLSPG (SEQ ID | | (SEQ ID | PVTKSFNR |
| | | S (SEQ ID | NO: 587) | | NO: 323) | GEC (SEQ |
| | | NO: 136) | | | | ID NO: |
| | | | | | | 415) |
| TA79 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDHYMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTISSG | KDTLMISRTPEVTCVVVDVSDEDGEV | 588) | LLIYGASTR | WKVDNALQ |
| | | GSYVYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | AAVYYCAKI | AVEWESNGQPENNYKTTPPVLDSDGS | | YNNWPPWTF | KHKVYACE |
| | | LKNGKYIYY | FFLYSKLTVDKSRWQQGNVFSCSVMH | | GQGTKVEIK | VTHQGLSS |
| | | FDYWGQGTL | EALHNHYTQKSLSLSPG (SEQ ID | | (SEQ ID | PVTKSFNR |
| | | VTVSS | NO: 587) | | NO: 323) | GEC (SEQ |
| | | (SEQ ID | | | | ID NO: |
| | | NO: 137) | | | | 415) |
| TA80 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDHYMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTISSG | KDTLMISRTPEVTCVVVDVSDEDGEV | 588) | LLIYGASTR | WKVDNALQ |
| | | GSYKYYADS | KFNWYVDGVEVHNAKTKPREEQYNST | | ATGIPARFS | SGNSQESV |
| | | AKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCAKI | AVEWESNGQPENNYKTTPPVLDSDGS | | YNNWPPWTF | KHKVYACE |
| | | LKNGNYIYY | FFLYSKLTVDKSRWQQGNVFSCSVMH | | GQGTKVEIK | VTHQGLSS |
| | | FDYWGQGTL | EALHNHYTQKSLSLSPG (SEQ ID | | (SEQ ID | PVTKSFNR |
| | | VTVSS | NO: 587) | | NO: 323) | GEC (SEQ |
| | | (SEQ ID | | | | ID NO: |
| | | NO: 138) | | | | 415) |
| TA81 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | TSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSRNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYLDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 139) | | | | ID NO: |
| | | | | | | 415) |
| TA82 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDHYMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTISSG | KDTLMISRTPEVTCVVVDVSDEDGEV | 588) | LLIYGASTR | WKVDNALQ |
| | | GSYVYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLHL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | AAVYYCAKI | AVEWESNGQPENNYKTTPPVLDSDGS | | YNNWPPWTF | KHKVYACE |
| | | LKNGKYIYY | FFLYSKLTVDKSRWQQGNVFSCSVMH | | GQGTKVEIK | VTHQGLSS |
| | | FDYWGQGTL | EALHNHYTQKSLSLSPG (SEQ ID | | (SEQ ID | PVTKSFNR |
| | | VTVSS | NO: 587) | | NO: 323) | GEC (SEQ |
| | | (SEQ ID | | | | ID NO: |
| | | NO: 140) | | | | 415) |
| TA83 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAAFGF | FPAVLQSSGLYSLSSVVTVPSSSLGT | TGVHS | ATLSCRASQ | QLKSGTAS |
| | (SEQ ID | TFTGYDMTW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAISWN | KDTLMISRTPEVTCVVVDVSDEDGEV | 588) | LLIYGASTR | WKVDNALQ |
| | | TGSTTYYAD | KFNWYVDGVEVHNAKTKPREEQYNST | | ATGIPARFS | SGNSQESV |
| | | SVKGRFTIS | YRVVSVLTVLHQDWLNGKEYKCKVSN | | GSGSGTEFT | TEQDSKDS |
| | | RDNSKNTLY | KALPRPIEKTISKAKGQPREPQVYTL | | LTISSLQSE | TYSLSSTL |
| | | LQMNSLRAE | PPSRDELTKNQVSLTCLVKGFYPSDI | | DFAVYYCQQ | TLSKADYE |
| | | DTAVYYCTR | AVEWESNGQPENNYKTTPPVLDSDGS | | YNNWPPWTF | KHKVYACE |
| | | GYDRKNYFE | FFLYSKLTVDKSRWQQGNVFSCSVMH | | GQGTKVEIK | VTHQGLSS |
| | | YWGQGTLVT | EALHNHYTQKSLSLSPG (SEQ ID | | (SEQ ID | PVTKSFNR |
| | | VSS (SEQ | NO: 587) | | NO: 323) | GEC (SEQ |
| | | ID NO: | | | | ID NO: |
| | | 141) | | | | 415) |
| TA84 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | PSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNPKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 142) | | | | ID NO: |
| | | | | | | 415) |
| TA85 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TSSDYWMSW | QTYICNVNHKPSNTKVDKKVEPKSCD | (SEQ ID | SVSSNLAWY | VVCLLNNF |
| | NO: | VRQTPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | PSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 143) | | | | ID NO: |
| | | | | | | 415) |
| TA86 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQTPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | PSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 144) | | | | ID NO: |
| | | | | | | 415) |
| TA87 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCVASGF | FPAVLQSSGLYSLSSVVTVPSSSLGT | TGVHS | ATLSCRASQ | QLKSGTAS |
| | (SEQ ID | TFSDYYMGW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAIGTI | KDTLMISRTPEVTCVVVDVSDEDGEV | 588) | LLIYGASTR | WKVDNALQ |
| | | VTYYADSVK | KFNWYVDGVEVHNAKTKPREEQYNST | | ATGIPARFS | SGNSQESV |
| | | GRFTISRDN | YRVVSVLTVLHQDWLNGKEYKCKVSN | | GSGSGTEFT | TEQDSKDS |
| | | SKNTLYLQM | KALPRPIEKTISKAKGQPREPQVYTL | | LTISSLQSE | TYSLSSTL |
| | | NSLRADDTA | PPSRDELTKNQVSLTCLVKGFYPSDI | | DFAVYYCQQ | TLSKADYE |
| | | VYYCARGIN | AVEWESNGQPENNYKTTPPVLDSDGS | | YNNWPPWTF | KHKVYACE |
| | | YVDDWGQGT | FFLYSKLTVDKSRWQQGNVFSCSVMH | | GQGTKVEIK | VTHQGLSS |
| | | LVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 145) | | | | ID NO: |
| | | | | | | 415) |
| TA88 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSSYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTIGSP | KDTLMISRTPEVTCVVVDVSDEDGEV | 588) | LLIYGASTR | WKVDNALQ |
| | | GDTYYADSV | KFNWYVDGVEVHNAKTKPREEQYNST | | ATGIPARFS | SGNSQESV |
| | | KGRFTISRD | YRVVSVLTVLHQDWLNGKEYKCKVSN | | GSGSGTEFT | TEQDSKDS |
| | | NSKNTLYLQ | KALPRPIEKTISKAKGQPREPQVYTL | | LTISSLQSE | TYSLSSTL |
| | | LNSLTAEDT | PPSRDELTKNQVSLTCLVKGFYPSDI | | DFAVYYCQQ | TLSKADYE |
| | | AVYFCATGY | AVEWESNGQPENNYKTTPPVLDSDGS | | YNNWPPWTF | KHKVYACE |
| | | AIFDYWGQG | FFLYSKLTVDKSRWQQGNVFSCSVMH | | GQGTKVEIK | VTHQGLSS |
| | | TLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 146) | | | | ID NO: |
| | | | | | | 415) |
| TA89 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCVASGF | FPAVLQSSGLYSLSSVVTVPSSSLGT | TGVHS | ATLSCRASQ | QLKSGTAS |
| | (SEQ ID | TFSDYYMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAIGTI | KDTLMISRTPEVTCVVVDVSDEDGEV | 588) | LLIYGASTR | WKVDNALQ |
| | | ITYYADSVK | KFNWYVDGVEVHNAKTKPREEQYNST | | ATGIPARFS | SGNSQESV |
| | | GRFTISRDN | YRVVSVLTVLHQDWLNGKEYKCKVSN | | GSGSGTEFT | TEQDSKDS |
| | | SKNTLYLQM | KALPRPIEKTISKAKGQPREPQVYTL | | LTISSLQSE | TYSLSSTL |
| | | NSLRADDTA | PPSRDELTKNQVSLTCLVKGFYPSDI | | DFAVYYCQQ | TLSKADYE |
| | | VYYCARGIN | AVEWESNGQPENNYKTTPPVLDSDGS | | YNNWPPWTF | KHKVYACE |
| | | FVDDWGQGT | FFLYSKLTVDKSRWQQGNVFSCSVMH | | GQGTKVEIK | VTHQGLSS |
| | | LVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 147) | | | | ID NO: |
| | | | | | | 415) |
| TA90 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSGYDMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAIGWK | KDTLMISRTPEVTCVVVDVSDEDGEV | 588) | LLIYGASTR | WKVDNALQ |
| | | SGSIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCAKG | AVEWESNGQPENNYKTTPPVLDSDGS | | YNNWPPWTF | KHKVYACE |
| | | YNLKNYFDY | FFLYSKLTVDKSRWQQGNVFSCSVMH | | GQGTKVEIK | VTHQGLSS |
| | | WGQGTLVTV | EALHNHYTQKSLSLSPG (SEQ ID | | (SEQ ID | PVTKSFNR |
| | | SS (SEQ | NO: 587) | | NO: 323) | GEC (SEQ |
| | | ID NO: | | | | ID NO: |
| | | 148) | | | | 415) |
| TA91 | MGWSCII | EVQLLESGG | ASTKGPSVFPLAPSSKSTSGGTAALG | MGWSCII | EIVMTQSPA | RTVAAPSV |
| | LFLVATA | DSVQPGESL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSSSAMHW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSATNND | KDTLMISRTPEVTCVVVDVSDEDGEV | 588) | LLIYGASTR | WKVDNALQ |
| | | GSITYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNGLRAED | PPSRDELTKNQVSLTCLVKGFYPSDI | | DFAVYYCQQ | TLSKADYE |
| | | TAVYYCARI | AVEWESNGQPENNYKTTPPVLDSDGS | | YNNWPPWTF | KHKVYACE |
| | | IYYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 149) | | | | ID NO: |
| | | | | | | 415) |
| TA92 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | IFSGYDMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAISWT | KDTLMISRTPEVTCVVVDVSDEDGEV | 588) | LLIYGASTR | WKVDNALQ |
| | | SGSIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCAKG | AVEWESNGQPENNYKTTPPVLDSDGS | | YNNWPPWTF | KHKVYACE |
| | | YNRNNYFDY | FFLYSKLTVDKSRWQQGNVFSCSVMH | | GQGTKVEIK | VTHQGLSS |
| | | WGQGTLVTV | EALHNHYTQKSLSLSPG (SEQ ID | | (SEQ ID | PVTKSFNR |
| | | SS (SEQ | NO: 587) | | NO: 323) | GEC (SEQ |
| | | ID NO: | | | | ID NO: |
| | | 150) | | | | 415) |
| TA93 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | GSSSRSYIY | KFNWYVDGVEVHNAKTKPREEQYNST | | ATGIPARFS | SGNSQESV |
| | | YADSVKGRF | YRVVSVLTVLHQDWLNGKEYKCKVSN | | GSGSGTEFT | TEQDSKDS |
| | | TISRDNSKN | KALPRPIEKTISKAKGQPREPQVYTL | | LTISSLQSE | TYSLSSTL |
| | | TLYLQMSSL | PPSRDELTKNQVSLTCLVKGFYPSDI | | DFAVYYCQQ | TLSKADYE |
| | | RAEDTAVYY | AVEWESNGQPENNYKTTPPVLDSDGS | | YNNWPPWTF | KHKVYACE |
| | | CTRGWAYFD | FFLYSKLTVDKSRWQQGNVFSCSVMH | | GQGTKVEIK | VTHQGLSS |
| | | YWGQGTLVT | EALHNHYTQKSLSLSPG (SEQ ID | | (SEQ ID | PVTKSFNR |
| | | VSS (SEQ | NO: 587) | | NO: 323) | GEC (SEQ |
| | | ID NO: | | | | ID NO: |
| | | 151) | | | | 415) |
| TA94 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSTYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSNIYSG | KDTLMISRTPEVTCVVVDVSDEDGEV | 588) | LLIYGASTR | WKVDNALQ |
| | | GSTIDYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCARG | AVEWESNGQPENNYKTTPPVLDSDGS | | YNNWPPWTF | KHKVYACE |
| | | WSYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 152) | | | | ID NO: |
| | | | | | | 415) |
| TA95 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | IFSGYDMSW | QTYICNVNHKPSNTKVDKKVEPKSCD | (SEQ ID | SVSSNLAWY | VVCLLNNF |
| | NO: | VRQAPGKGP | KTHTCPPCPAPDLLGDDSVFLFPPKP | NO: | QQKPGQAPR | YPREAKVQ |
| | 588) | EWVSAITWD | KDTLMISRTPEVTCVVVDVSDEDGEV | 588) | LLIYGASTR | WKVDNALQ |
| | | SGSTYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QTNSLRAED | PPSRDELTKNQVSLTCLVKGFYPSDI | | DFAVYYCQQ | TLSKADYE |
| | | TAVYYCAKG | AVEWESNGQPENNYKTTPPVLDSDGS | | YNNWPPWTF | KHKVYACE |
| | | YDRNNYFEY | FFLYSKLTVDKSRWQQGNVFSCSVMH | | GQGTKVEIK | VTHQGLSS |
| | | WGQGTLVTV | EALHNHYTQKSLSLSPG (SEQ ID | | (SEQ ID | PVTKSFNR |
| | | SS (SEQ | NO: 587) | | NO: 323) | GEC (SEQ |
| | | ID NO: | | | | ID NO: |
| | | 153) | | | | 415) |
| TA96 | MGWSCII | EVQLLESGG | ASTKGPSVFPLAPSSKSTSGGTAALG | MGWSCII | EIVMTQSPA | RTVAAPSV |
| | LFLVATA | GLIQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | NFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWISAIYSG | KDTLMISRTPEVTCVVVDVSDEDGEV | 588) | LLIYGASTR | WKVDNALQ |
| | | GYIYYADSV | KFNWYVDGVEVHNAKTKPREEQYNST | | ATGIPARFS | SGNSQESV |
| | | KGRFTISRD | YRVVSVLTVLHQDWLNGKEYKCKVSN | | GSGSGTEFT | TEQDSKDS |
| | | NSKNTLYLQ | KALPRPIEKTISKAKGQPREPQVYTL | | LTISSLQSE | TYSLSSTL |
| | | MNSLRAEDT | PPSRDELTKNQVSLTCLVKGFYPSDI | | DFAVYYCQQ | TLSKADYE |
| | | AVYYCTRGW | AVEWESNGQPENNYKTTPPVLDSDGS | | YNNWPPWTF | KHKVYACE |
| | | SYCDYWGQG | FFLYSKLTVDKSRWQQGNVFSCSVMH | | GQGTKVEIK | VTHQGLSS |
| | | TLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 154) | | | | ID NO: |
| | | | | | | 415) |
| TA97 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | NFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAIYSG | KDTLMISRTPEVTCVVVDVSDEDGEV | 588) | LLIYGASTR | WKVDNALQ |
| | | GYIYYADSV | KFNWYVDGVEVHNAKTKPREEQYNST | | ATGIPARFS | SGNSQESV |
| | | KGRFTISRD | YRVVSVLTVLHQDWLNGKEYKCKVSN | | GSGSGTEFT | TEQDSKDS |
| | | NSKNTLYLQ | KALPRPIEKTISKAKGQPREPQVYTL | | LTISSLQSE | TYSLSSTL |
| | | MNSLRAEDT | PPSRDELTKNQVSLTCLVKGFYPSDI | | DFAVYYCQQ | TLSKADYE |
| | | AVYYCARGW | AVEWESNGQPENNYKTTPPVLDSDGS | | YNNWPPWTF | KHKVYACE |
| | | SYCDSWGQG | FFLYSKLTVDKSRWQQGNVFSCSVMH | | GQGTKVEIK | VTHQGLSS |
| | | TLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 155) | | | | ID NO: |
| | | | | | | 415) |
| TA98 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDHYMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTISSG | KDTLMISRTPEVTCVVVDVSDEDGEV | 588) | LLIYGASTR | WKVDNALQ |
| | | GNYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DSSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCARI | AVEWESNGQPENNYKTTPPVLDSDGS | | YNNWPPWTF | KHKVYACE |
| | | LKNGKYIYY | FFLYSKLTVDKSRWQQGNVFSCSVMH | | GQGTKVEIK | VTHQGLSS |
| | | FDYWGQGTL | EALHNHYTQKSLSLSPG (SEQ ID | | (SEQ ID | PVTKSFNR |
| | | VTVSS | NO: 587) | | NO: 323) | GEC (SEQ |
| | | (SEQ ID | | | | ID NO: |
| | | NO: 156) | | | | 415) |
| TA99 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSS | EELQANKA |
| | (SEQ ID | TFSSYDMSW | QTYICNVNHKPSNTKVDKKVEPKSCD | (SEQ ID | NIGSKYVSW | TLVCLISD |
| | NO: | VRQAPGEGL | KTHTCPPCPAPDLLGDDSVFLFPPKP | NO: | YQQLPGTAP | FYPGAVTV |
| | 588) | EWVSAISGS | KDTLMISRTPEVTCVVVDVSDEDGEV | 588) | KLLIYKDDQ | AWKADSSP |
| | | GVSAYYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCA | SLTPEQWK |
| | | TAIYYCAKD | AVEWESNGQPENNYKTTPPVLDSDGS | | AWDGSLNGW | SHRSYSCQ |
| | | GLFFDYWGL | FFLYSKLTVDKSRWQQGNVFSCSVMH | | VFGGGTKLT | VTHEGSTV |
| | | GTLVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 157) | | | 324) | ID NO: |
| | | | | | | 416) |
| TA100 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | VSGTPGQRV | VTLFPPSS |
| | TGVHS | RLSCAASGF | FPAVLQSSGLYSLSSVVTVPSSSLGT | TGVHS | TISCSGSNS | EELQANKA |
| | (SEQ ID | NFSSYDMNW | QTYICNVNHKPSNTKVDKKVEPKSCD | (SEQ ID | NIGGYYVSW | TLVCLISD |
| | NO: | VRQAPGEGL | KTHTCPPCPAPDLLGDDSVFLFPPKP | NO: | YQQVPGTAP | FYPGAVTV |
| | 588) | QWVATITAA | KDTLMISRTPEVTCVVVDVSDEDGEV | 588) | KLLIYKNFQ | AWKADSSP |
| | | GDITYYADS | KFNWYVDGVEVHNAKTKPREEQYNST | | RPSGVPDRF | VKAGVETT |
| | | VRGRFAISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | GLSSLRAED | PPSRDELTKNQVSLTCLVKGFYPSDI | | EDEADYYCA | SLTPEQWK |
| | | TAIYYCGRE | AVEWESNGQPENNYKTTPPVLDSDGS | | SWDGSLNGW | SHRSYSCQ |
| | | PWNSFSDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 158) | | | 325) | ID NO: |
| | | | | | | 416) |
| TA101 | MGWSCII | DVQLVESGG | ASTKGPSVFPLAPSSKSTSGGTAALG | MGWSCII | QSVLTQPPS | GQPKAAPS |
| | LFLVATA | GVARPGEFL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFRDYDMGW | QTYICNVNHKPSNTKVDKKVEPKSCD | (SEQ ID | NIGTRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVSSISVS | KDTLMISRTPEVTCVVVDVSDEDGEV | 588) | KLLIYKNLQ | AWKADSSP |
| | | GTTYYADSV | KFNWYVDGVEVHNAKTKPREEQYNST | | RPSGVPDRF | VKAGVETT |
| | | KGRFTISRD | YRVVSVLTVLHQDWLNGKEYKCKVSN | | SGSKSGTSA | TPSKQSNN |
| | | NSENTLYLQ | KALPRPIEKTISKAKGQPREPQVYTL | | SLAISGLQS | KYAASSYL |
| | | MNSLTAEDT | PPSRDELTKNQVSLTCLVKGFYPSDI | | EDEADYYCA | SLTPEQWK |
| | | AVYYCGRED | AVEWESNGQPENNYKTTPPVLDSDGS | | AWDERLNGW | SHRSYSCQ |
| | | TLFHYWGLG | FFLYSKLTVDKSRWQQGNVFSCSVMH | | VFGGGTKLT | VTHEGSTV |
| | | TLVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 159) | | | 326) | ID NO: |
| | | | | | | 416) |
| TA102 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSS | EELQANKA |
| | (SEQ ID | TFRNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DDTTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 160) | | | 327) | ID NO: |
| | | | | | | 416) |
| TA103 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFSNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVSSISPS | KDTLMISRTPEVTCVVVDVSDEDGEV | 588) | KLLIYRNFE | AWKADSSP |
| | | AYSAYYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | EMNSLRAED | PPSRDELTKNQVSLTCLVKGFYPSDI | | EDEADYYCA | SLTPEQWK |
| | | TAVYYCAKT | AVEWESNGQPENNYKTTPPVLDSDGS | | SWDESLNGW | SHRSYSCQ |
| | | SGNINYGLD | FFLYSKLTVDKSRWQQGNVFSCSVMH | | VFGGGTKLT | VTHEGSTV |
| | | YWGLGTLVT | EALHNHYTQKSLSLSPG (SEQ ID | | VL (SEQ | EKTVAPTE |
| | | VSS (SEQ | NO: 587) | | ID NO: | CS (SEQ |
| | | ID NO: | | | 328) | ID NO: |
| | | 161) | | | | 416) |
| TA104 | MGWSCII | DVQLVESGG | ASTKGPSVFPLAPSSKSTSGGTAALG | MGWSCII | QSVLTQPPS | GQPKAAPS |
| | LFLVATA | GVARPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFRDYDMGW | QTYICNVNHKPSNTKVDKKVEPKSCD | (SEQ ID | NIGTRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVSSISVS | KDTLMISRTPEVTCVVVDVSDEDGEV | 588) | KLLIYKNLQ | AWKADSSP |
| | | GTTYYADSV | KFNWYVDGVEVHNAKTKPREEQYNST | | RPSGVPDRF | VKAGVETT |
| | | KGRFTISRD | YRVVSVLTVLHQDWLNGKEYKCKVSN | | SGSKSGTSA | TPSKQSNN |
| | | NSENTLYLQ | KALPRPIEKTISKAKGQPREPQVYTL | | SLAISGLQS | KYAASSYL |
| | | MNSLTAEDT | PPSRDELTKNQVSLTCLVKGFYPSDI | | EDEADYYCA | SLTPEQWK |
| | | AVYYCGRED | AVEWESNGQPENNYKTTPPVLDSDGS | | AWDERLNGW | SHRSYSCQ |
| | | TLFHYWGLG | FFLYSKLTVDKSRWQQGNVFSCSVMH | | VFGGGTKLT | VTHEGSTV |
| | | TLVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 162) | | | 326) | ID NO: |
| | | | | | | 416) |
| TA105 | MGWSCII | EVQLLESGG | ASTKGPSVFPLAPSSKSTSGGTAALG | MGWSCII | EIVMTQSPA | RTVAAPSV |
| | LFLVATA | DLLQPGGSL | CLVKDYFPEPVTVSWNSGALTSGVHT | LFLVATA | TLSVSPGER | FIFPPSDE |
| | TGVHS | RLSCSASGF | FPAVLQSSGLYSLSSVVTVPSSSLGT | TGVHS | ATLSCRASQ | QLKSGTAS |
| | (SEQ ID | DFSSYYMTW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVAAITSL | KDTLMISRTPEVTCVVVDVSDEDGEV | 588) | LLIYGASTR | WKVDNALQ |
| | | GYTTYYANS | KFNWYVDGVEVHNAKTKPREEQYNST | | ATGIPARFS | SGNSQESV |
| | | VEGRFTISR | YRVVSVLTVLHQDWLNGKEYKCKVSN | | GSGSGTEFT | TEQDSKDS |
| | | DNSENTLYL | KALPRPIEKTISKAKGQPREPQVYTL | | LTISSLQSE | TYSLSSTL |
| | | EMNSLRAED | PPSRDELTKNQVSLTCLVKGFYPSDI | | DFAVYYCQQ | TLSKADYE |
| | | TAVYYCATT | AVEWESNGQPENNYKTTPPVLDSDGS | | YNNWPPWTF | KHKVYACE |
| | | HARGSRYFD | FFLYSKLTVDKSRWQQGNVFSCSVMH | | GQGTKVEIK | VTHQGLSS |
| | | YWGQGTLVT | EALHNHYTQKSLSLSPG (SEQ ID | | (SEQ ID | PVTKSFNR |
| | | VSS (SEQ | NO: 587) | | NO: 323) | GEC (SEQ |
| | | ID NO: | | | | ID NO: |
| | | 163) | | | | 415) |
| TA106 | MGWSCII | EVQLLESGG | ASTKGPSVFPLAPSSKSTSGGTAALG | MGWSCII | EIVMTQSPA | RTVAAPSV |
| | LFLVATA | GLLQPGGSL | CLVKDYFPEPVTVSWNSGALTSGVHT | LFLVATA | TLSVSPGER | FIFPPSDE |
| | TGVHS | RLSCSASGF | FPAVLQSSGLYSLSSVVTVPSSSLGT | TGVHS | ATLSCRASQ | QLKSGTAS |
| | (SEQ ID | DFSSYYMTW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVAAITSL | KDTLMISRTPEVTCVVVDVSDEDGEV | 588) | LLIYGASTR | WKVDNALQ |
| | | GYTTYYANS | KFNWYVDGVEVHNAKTKPREEQYNST | | ATGIPARFS | SGNSQESV |
| | | VEGRFTISR | YRVVSVLTVLHQDWLNGKEYKCKVSN | | GSGSGTEFT | TEQDSKDS |
| | | DNSENTLYL | KALPRPIEKTISKAKGQPREPQVYTL | | LTISSLQSE | TYSLSSTL |
| | | EMNSLRAED | PPSRDELTKNQVSLTCLVKGFYPSDI | | DFAVYYCQQ | TLSKADYE |
| | | TAVYYCATT | AVEWESNGQPENNYKTTPPVLDSDGS | | YNNWPPWTF | KHKVYACE |
| | | HARGSRYFD | FFLYSKLTVDKSRWQQGNVFSCSVMH | | GQGTKVEIK | VTHQGLSS |
| | | YWGQGTLVT | EALHNHYTQKSLSLSPG (SEQ ID | | (SEQ ID | PVTKSFNR |
| | | VSS (SEQ | NO: 587) | | NO: 323) | GEC (SEQ |
| | | ID NO: | | | | ID NO: |
| | | 164) | | | | 415) |
| TA107 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | SFSDYHMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSISWN | KDTLMISRTPEVTCVVVDVSDEDGEV | 588) | LLIYGASTR | WKVDNALQ |
| | | RGTTHYADS | KFNWYVDGVEVHNAKTKPREEQYNST | | ATGIPARFS | SGNSQESV |
| | | VRGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRM | AVEWESNGQPENNYKTTPPVLDSDGS | | YNNWPPWTF | KHKVYACE |
| | | QVYLMTSYF | FFLYSKLTVDKSRWQQGNVFSCSVMH | | GQGTKVEIK | VTHQGLSS |
| | | DYWGQGTLV | EALHNHYTQKSLSLSPG (SEQ ID | | (SEQ ID | PVTKSFNR |
| | | TVSS (SEQ | NO: 587) | | NO: 323) | GEC (SEQ |
| | | ID NO: | | | | ID NO: |
| | | 165) | | | | 415) |
| TA108 | MGWSCII | EVQLLESGG | ASTKGPSVFPLAPSSKSTSGGTAALG | MGWSCII | EIVMTQSPA | RTVAAPSV |
| | LFLVATA | GSVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYYMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAISWN | KDTLMISRTPEVTCVVVDVSDEDGEV | 588) | LLIYGASTR | WKVDNALQ |
| | | NGRTYYADS | KFNWYVDGVEVHNAKTKPREEQYNST | | ATGIPARFS | SGNSQESV |
| | | VKGRFTISR | YRVVSVLIVLHQDWLNGKEYKCKVSN | | GSGSGTEFT | TEQDSKDS |
| | | DDSKNTLYL | KALPRPIEKTISKAKGQPREPQVYTL | | LTISSLQSE | TYSLSSTL |
| | | QMNSLTAED | PPSRDELTKNQVSLTCLVKGFYPSDI | | DFAVYYCQQ | TLSKADYE |
| | | TAVYYCAKM | AVEWESNGQPENNYKTTPPVLDSDGS | | YNNWPPWTF | KHKVYACE |
| | | LVYLMTSYF | FFLYSKLTVDKSRWQQGNVFSCSVMH | | GQGTKVEIK | VTHQGLSS |
| | | DSWGQGTLV | EALHNHYTQKSLSLSPG (SEQ ID | | (SEQ ID | PVTKSFNR |
| | | TVSS (SEQ | NO: 587) | | NO: 323) | GEC (SEQ |
| | | ID NO: | | | | ID NO: |
| | | 166) | | | | 415) |
| TA109 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | SFSDYHMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTISWN | KDTLMISRTPEVTCVVVDVSDEDGEV | 588) | LLIYGASTR | WKVDNALQ |
| | | RGTTHYADS | KFNWYVDGVEVHNAKTKPREEQYNST | | ATGIPARFS | SGNSQESV |
| | | VKGRFTISR | YRVVSVLIVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRM | AVEWESNGQPENNYKTTPPVLDSDGS | | YNNWPPWTF | KHKVYACE |
| | | QVYLMTSYF | FFLYSKLTVDKSRWQQGNVFSCSVMH | | GQGTKVEIK | VTHQGLSS |
| | | DYWGQGTLV | EALHNHYTQKSLSLSPG (SEQ ID | | (SEQ ID | PVTKSFNR |
| | | TVSS (SEQ | NO: 587) | | NO: 323) | GEC (SEQ |
| | | ID NO: | | | | ID NO: |
| | | 167) | | | | 415) |
| TA110 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | SFSDYHMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTISWN | KDTLMISRTPEVTCVVVDVSDEDGEV | 588) | LLIYGASTR | WKVDNALQ |
| | | RGTTHYADS | KFNWYVDGVEVHNAKTKPREEQYNST | | ATGIPARFS | SGNSQESV |
| | | VKGRLTISR | YRVVSVLIVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRM | AVEWESNGQPENNYKTTPPVLDSDGS | | YNNWPPWTF | KHKVYACE |
| | | QVYLMTSYF | FFLYSKLTVDKSRWQQGNVFSCSVMH | | GQGTKVEIK | VTHQGLSS |
| | | DYWGQGTLV | EALHNHYTQKSLSLSPG (SEQ ID | | (SEQ ID | PVTKSFNR |
| | | TVSS (SEQ | NO: 587) | | NO: 323) | GEC (SEQ |
| | | ID NO: | | | | ID NO: |
| | | 168) | | | | 415) |
| TA111 | MGWSCII | EVQLLESGG | ASTKGPSVFPLAPSSKSTSGGTAALG | MGWSCII | EIVMTQSPA | RTVAAPSV |
| | LFLVATA | GSVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYYMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAISWN | KDTLMISRTPEVTCVVVDVSDEDGEV | 588) | LLIYGASTR | WKVDNALQ |
| | | NGRMYYADS | KFNWYVDGVEVHNAKTKPREEQYNST | | ATGIPARFS | SGNSQESV |
| | | VKGRFTISR | YRVVSVLIVLHQDWLNGKEYKCKVSN | | GSGSGTEFT | TEQDSKDS |
| | | DDSKNTLYL | KALPRPIEKTISKAKGQPREPQVYTL | | LTISSLQSE | TYSLSSTL |
| | | QMNSLTAED | PPSRDELTKNQVSLTCLVKGFYPSDI | | DFAVYYCQQ | TLSKADYE |
| | | TAVYYCAKM | AVEWESNGQPENNYKTTPPVLDSDGS | | YNNWPPWTF | KHKVYACE |
| | | LVYLMTSYF | FFLYSKLTVDKSRWQQGNVFSCSVMH | | GQGTKVEIK | VTHQGLSS |
| | | DSWGQGTLV | EALHNHYTQKSLSLSPG (SEQ ID | | (SEQ ID | PVTKSFNR |
| | | TVSS (SEQ | NO: 587) | | NO: 323) | GEC (SEQ |
| | | ID NQ | | | | ID NO: |
| | | 169) | | | | 415) |
| TA112 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCVGSGF | FPAVLQSSGLYSLSSVVTVPSSSLGT | TGVHS | ATLSCRASQ | QLKSGTAS |
| | (SEQ ID | VFDEYGIHW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVAAIDWS | KDTLMISRTPEVTCVVVDVSDEDGEV | 588) | LLIYGASTR | WKVDNALQ |
| | | GNRTYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTAYL | KALPRPIEKTISKAKGQPREPQVYTL | | LTISSLQSE | TYSLSSTL |
| | | QMNSLTAED | PPSRDELTKNQVSLTCLVKGFYPSDI | | DFAVYYCQQ | TLSKADYE |
| | | TAVYYCAKF | AVEWESNGQPENNYKTTPPVLDSDGS | | YNNWPPWTF | KHKVYACE |
| | | RWRDFYFEY | FFLYSKLTVDKSRWQQGNVFSCSVMH | | GQGTKVEIK | VTHQGLSS |
| | | WGQGTLVTV | EALHNHYTQKSLSLSPG (SEQ ID | | (SEQ ID | PVTKSFNR |
| | | SS (SEQ | NO: 587) | | NO: 323) | GEC (SEQ |
| | | ID NO: | | | | ID NO: |
| | | 170) | | | | 415) |
| TA113 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | SFSDYHMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSISWN | KDTLMISRTPEVTCVVVDVSDEDGEV | 588) | LLIYGASTR | WKVDNALQ |
| | | RGTTHYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMSSLRAED | PPSRDELTKNQVSLTCLVKGFYPSDI | | DFAVYYCQQ | TLSKADYE |
| | | TAVYYCTRM | AVEWESNGQPENNYKTTPPVLDSDGS | | YNNWPPWTF | KHKVYACE |
| | | QVYLMTSYF | FFLYSKLTVDKSRWQQGNVFSCSVMH | | GQGTKVEIK | VTHQGLSS |
| | | DYWGQGTLV | EALHNHYTQKSLSLSPG (SEQ ID | | (SEQ ID | PVTKSFNR |
| | | TVSS (SEQ | NO: 587) | | NO: 323) | GEC (SEQ |
| | | ID NO: | | | | ID NO: |
| | | 171) | | | | 415) |
| TA114 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCVGSGF | FPAVLQSSGLYSLSSVVTVPSSSLGT | TGVHS | ATLSCRASQ | QLKSGTAS |
| | (SEQ ID | VFDEYGIHW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVAAIDWS | KDTLMISRTPEVTCVVVDVSDEDGEV | 588) | LLIYGASTR | WKVDNALQ |
| | | GNRTYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTVYL | KALPRPIEKTISKAKGQPREPQVYTL | | LTISSLQSE | TYSLSSTL |
| | | QMNSLTAED | PPSRDELTKNQVSLTCLVKGFYPSDI | | DFAVYYCQQ | TLSKADYE |
| | | TAVYYCAKF | AVEWESNGQPENNYKTTPPVLDSDGS | | YNNWPPWTF | KHKVYACE |
| | | RWRDFYFEY | FFLYSKLTVDKSRWQQGNVFSCSVMH | | GQGTKVEIK | VTHQGLSS |
| | | WGQGTLVTV | EALHNHYTQKSLSLSPG (SEQ ID | | (SEQ ID | PVTKSFNR |
| | | SS (SEQ | NO: 587) | | NO: 323) | GEC (SEQ |
| | | ID NO: | | | | ID NO: |
| | | 172) | | | | 415) |
| TA115 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCVGSGF | FPAVLQSSGLYSLSSVVTVPSSSLGT | TGVHS | ATLSCRASQ | QLKSGTAS |
| | (SEQ ID | VFDEYGIHW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVAAIDWS | KDTLMISRTPEVTCVVVDVSDEDGEV | 588) | LLIYGASTR | WKVDNALQ |
| | | GNRTYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSRNTVYL | KALPRPIEKTISKAKGQPREPQVYTL | | LTISSLQSE | TYSLSSTL |
| | | QMNSLTAED | PPSRDELTKNQVSLTCLVKGFYPSDI | | DFAVYYCQQ | TLSKADYE |
| | | TAVYYCAKF | AVEWESNGQPENNYKTTPPVLDSDGS | | YNNWPPWTF | KHKVYACE |
| | | RRREFYFEY | FFLYSKLTVDKSRWQQGNVFSCSVMH | | GQGTKVEIK | VTHQGLSS |
| | | WGQGTLVTV | EALHNHYTQKSLSLSPG (SEQ ID | | (SEQ ID | PVTKSFNR |
| | | SS (SEQ | NO: 587) | | NO: 323) | GEC (SEQ |
| | | ID NO: | | | | ID NO: |
| | | 173) | | | | 415) |
| TA116 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | SFSDYHMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSISWN | KDTLMISRTPEVTCVVVDVSDEDGEV | 588) | LLIYGASTR | WKVDNALQ |
| | | RGTTHYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRM | AVEWESNGQPENNYKTTPPVLDSDGS | | YNNWPPWTF | KHKVYACE |
| | | QVYLMTSYF | FFLYSKLTVDKSRWQQGNVFSCSVMH | | GQGTKVEIK | VTHQGLSS |
| | | DYWGQGTLV | EALHNHYTQKSLSLSPG (SEQ ID | | (SEQ ID | PVTKSFNR |
| | | TVSS (SEQ | NO: 587) | | NO: 323) | GEC (SEQ |
| | | ID NO: | | | | ID NO: |
| | | 174) | | | | 415) |
| TA117 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSS | EELQANKA |
| | (SEQ ID | TLSDYWMSW | QTYICNVNHKPSNTKVDKKVEPKSCD | (SEQ ID | NIRNNYVSW | TLVCLISD |
| | NO: | VRQAPGKGL | KTHTCPPCPAPDLLGDDSVFLFPPKP | NO: | YQQLPGTAP | FYPGAVTV |
| | 588) | EWVSDISWS | KDTLMISRTPEVTCVVVDVSDEDGEV | 588) | KLLIYKYNQ | AWKADSSP |
| | | AGDTYYYAD | KFNWYVDGVEVHNAKTKPREEQYNST | | RPSGVPDRF | VKAGVETT |
| | | SVKGRFTIS | YRVVSVLTVLHQDWLNGKEYKCKVSN | | SGSKSGTSA | TPSKQSNN |
| | | RDNSKNTLY | KALPRPIEKTISKAKGQPREPQVYTL | | SLAISGLQS | KYAASSYL |
| | | LQMNSLRAE | PPSRDELTKNQVSLTCLVKGFYPSDI | | EDEADYYCG | SLTPEQWK |
| | | DTAVYYCAK | AVEWESNGQPENNYKTTPPVLDSDGS | | TWDDSLNGF | SHRSYSCQ |
| | | YQRNGGYSF | FFLYSKLTVDKSRWQQGNVFSCSVMH | | VFGGGTKLT | VTHEGSTV |
| | | DYWGQGTLV | EALHNHYTQKSLSLSPG (SEQ ID | | VL (SEQ | EKTVAPTE |
| | | TVSS (SEQ | NO: 587) | | ID NO: | CS (SEQ |
| | | ID NO: | | | 329) | ID NO: |
| | | 175) | | | | 416) |
| TA118 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSS | EELQANKA |
| | (SEQ ID | TFDDYGMSW | QTYICNVNHKPSNTKVDKKVEPKSCD | (SEQ ID | NIGNNYVSW | TLVCLISD |
| | NO: | VRQAPGKGL | KTHTCPPCPAPDLLGDDSVFLFPPKP | NO: | YQQLPGTAP | FYPGAVTV |
| | 588) | EWVSAISWS | KDTLMISRTPEVTCVVVDVSDEDGEV | 588) | KLLIYKDDQ | AWKADSSP |
| | | GGRTYYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DDSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCA | SLTPEQWK |
| | | TAVYYCTRD | AVEWESNGQPENNYKTTPPVLDSDGS | | ARVDTLNVW | SHRSYSCQ |
| | | ITILTTYYF | FFLYSKLTVDKSRWQQGNVFSCSVMH | | VFGGGTKLT | VTHEGSTV |
| | | DYWGQGTLV | EALHNHYTQKSLSLSPG (SEQ ID | | VL (SEQ | EKTVAPTE |
| | | TVSS (SEQ | NO: 587) | | ID NO: | CS (SEQ |
| | | ID NO: | | | 330) | ID NO: |
| | | 176) | | | | 416) |
| TA119 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSS | EELQANKA |
| | (SEQ ID | MENGSIMQW | QTYICNVNHKPSNTKVDKKVEPKSCD | (SEQ ID | NIRNNYVSW | TLVCLISD |
| | NO: | VRQAPGKGL | KTHTCPPCPAPDLLGDDSVFLFPPKP | NO: | YQQLPGTAP | FYPGAVTV |
| | 588) | EWVAGISDN | KDTLMISRTPEVTCVVVDVSDEDGEV | 588) | KLLIYRNSQ | AWKADSSP |
| | | GGTSYPDFV | KFNWYVDGVEVHNAKTKPREEQYNST | | RPSGVPDRF | VKAGVETT |
| | | KGRFTISRD | YRVVSVLTVLHQDWLNGKEYKCKVSN | | SGSKSGTSA | TPSKQSNN |
| | | NSKNTVYLQ | KALPRPIEKTISKAKGQPREPQVYTL | | SLAISGLQS | KYAASSYL |
| | | MNSLRAEDT | PPSRDELTKNQVSLTCLVKGFYPSDI | | EDEADYYCA | SLTPEQWK |
| | | AVYYCVKDI | AVEWESNGQPENNYKTTPPVLDSDGS | | AWDDSLNGW | SHRSYSCQ |
| | | DGYYFDYWG | FFLYSKLTVDKSRWQQGNVFSCSVMH | | VFGGGTKLT | VTHEGSTV |
| | | QGTLVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 177) | | | 331) | ID NO: |
| | | | | | | 416) |
| TA120 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSP | EELQANKA |
| | (SEQ ID | TLSDYWMSW | QTYICNVNHKPSNTKVDKKVEPKSCD | (SEQ ID | NIRNNYVSW | TLVCLISD |
| | NO: | VRQAPGKGL | KTHTCPPCPAPDLLGDDSVFLFPPKP | NO: | YQQLPGTAP | FYPGAVTV |
| | 588) | EWVSDISWS | KDTLMISRTPEVTCVVVDVSDEDGEV | 588) | KLLIYKYNQ | AWKADSSP |
| | | AGDTYYYAD | KFNWYVDGVEVHNAKTKPREEQYNST | | RPSGVPDRF | VKAGVETT |
| | | SVKGRFTIS | YRVVSVLIVLHQDWLNGKEYKCKVSN | | SGSKSGTSA | TPSKQSNN |
| | | RDNSKNTLY | KALPRPIEKTISKAKGQPREPQVYTL | | SLAISGLQS | KYAASSYL |
| | | LQMNSLRAE | PPSRDELTKNQVSLTCLVKGFYPSDI | | EDEADYYCG | SLTPEQWK |
| | | DTAVYYCAK | AVEWESNGQPENNYKTTPPVLDSDGS | | TWDDSLNGF | SHRSYSCQ |
| | | YQRNGGYSF | FFLYSKLTVDKSRWQQGNVFSCSVMH | | VFGGGTKLT | VTHEGSTV |
| | | DYWGQGTLV | EALHNHYTQKSLSLSPG (SEQ ID | | VL (SEQ | EKTVAPTE |
| | | TVSS (SEQ | NO: 587) | | ID NO: | CS (SEQ |
| | | ID NO: | | | 332) | ID NO: |
| | | 175) | | | | 416) |
| TA121 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAVSGF | FPAVLQSSGLYSLSSVVTVPSSSLGT | TGVHS | TISCSGSTS | EELQANKA |
| | (SEQ ID | TFSGSAMSW | QTYICNVNHKPSNTKVDKKVEPKSCD | (SEQ ID | NIGTNRVYW | TLVCLISD |
| | NO: | VRQAPGKGR | KTHTCPPCPAPDLLGDDSVFLFPPKP | NO: | YQRLPGTAP | FYPGAVTV |
| | 588) | EYVAGISSN | KDTLMISRTPEVTCVVVDVSDEDGEV | 588) | KLLIYRDQE | AWKADSSP |
| | | GGTTYFTDS | KFNWYVDGVEVHNAKTKPREEQYNST | | RPSGVPDRF | VKAGVETT |
| | | MKGRFTISR | YRVVSVLIVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMSSLRAED | PPSRDELTKNQVSLTCLVKGFYPSDI | | EDEADYYCA | SLTPEQWK |
| | | TAVYYCARG | AVEWESNGQPENNYKTTPPVLDSDGS | | SWDDSLNAW | SHRSYSCQ |
| | | GYNWNIYID | FFLYSKLTVDKSRWQQGNVFSCSVMH | | VFGGGTKLT | VTHEGSTV |
| | | YWGQGTLVT | EALHNHYTQKSLSLSPG (SEQ ID | | VL (SEQ | EKTVAPTE |
| | | VSS (SEQ | NO: 587) | | ID NO: | CS (SEQ |
| | | ID NO: | | | 333) | ID NO: |
| | | 178) | | | | 416) |
| TA122 | MGWSCII | EVQLLESGG | ASTKGPSVFPLAPSSKSTSGGTAALG | MGWSCII | QSVLTQPPS | GQPKAAPS |
| | LFLVATA | AFVQPGRSL | CLVKDYFPEPVTVSWNSGALTSGVHT | LFLVATA | ASGAPGQRV | VTLFPPSS |
| | TGVHS | GLSCAASGF | FPAVLQSSGLYSLSSVVTVPSSSLGT | TGVHS | TISCSGSYS | EELQANKA |
| | (SEQ ID | TFSDYYMSW | QTYICNVNHKPSNTKVDKKVEPKSCD | (SEQ ID | NIGNSYVSW | TLVCLISD |
| | NO: | VRQAPGKGL | KTHTCPPCPAPDLLGDDSVFLFPPKP | NO: | YQQPPGTAP | FYPGAVTV |
| | 588) | EWVSVISNS | KDTLMISRTPEVTCVVVDVSDEDGEV | 588) | KLLIYLNSQ | AWKADSSP |
| | | GGSTYYTDS | KFNWYVDGVEVHNAKTKPREEQYNST | | RPSGVPDRF | VKAGVETT |
| | | VKGRFTISR | YRVVSVLIVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCA | SLTPEQWK |
| | | TAVYYCTKD | AVEWESNGQPENNYKTTPPVLDSDGS | | AWDDLNVWV | SHRSYSCQ |
| | | IGMTYFDYW | FFLYSKLTVDKSRWQQGNVFSCSVMH | | FGGGTKLTV | VTHEGSTV |
| | | GQGTLVTVS | EALHNHYTQKSLSLSPG (SEQ ID | | L (SEQ ID | EKTVAPTE |
| | | S (SEQ ID | NO: 587) | | NO: 334) | CS (SEQ |
| | | NO: 179) | | | | ID NO: |
| | | | | | | 416) |
| TA123 | MGWSCII | EVQLLESGG | ASTKGPSVFPLAPSSKSTSGGTAALG | MGWSCII | QSVLTQPPS | GQPKAAPS |
| | LFLVATA | ALVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSYS | EELQANKA |
| | (SEQ ID | TFSDYYMSW | QTYICNVNHKPSNTKVDKKVEPKSCD | (SEQ ID | NIGNSYVSW | TLVCLISD |
| | NO: | VRQAPGKGL | KTHTCPPCPAPDLLGDDSVFLFPPKP | NO: | YQQLPGTAP | FYPGAVTV |
| | 588) | EWVSVISNS | KDTLMISRTPEVTCVVVDVSDEDGEV | 588) | KLLIYLNSQ | AWKADSSP |
| | | GGSTYYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLRS | KYAASSYL |
| | | QMNSLRAED | PPSRDELTKNQVSLTCLVKGFYPSDI | | EDEADYYCA | SLTPEQWK |
| | | TAVYYCTKD | AVEWESNGQPENNYKTTPPVLDSDGS | | AWDDLNVWV | SHRSYSCQ |
| | | IGMTYFDYW | FFLYSKLTVDKSRWQQGNVFSCSVMH | | FGGGTKLTV | VTHEGSTV |
| | | GQGTLVTVS | EALHNHYTQKSLSLSPG (SEQ ID | | L (SEQ ID | EKTVAPTE |
| | | S (SEQ ID | NO: 587) | | NO: 335) | CS (SEQ |
| | | NO: 180) | | | | ID NO: |
| | | | | | | 416) |
| TA124 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSRS | EELQANKA |
| | (SEQ ID | DFSGSPMTW | QTYICNVNHKPSNTKVDKKVEPKSCD | (SEQ ID | NIGTKYVSW | TLVCLISD |
| | NO: | VRQAPGKGL | KTHTCPPCPAPDLLGDDSVFLFPPKP | NO: | YQQLPGTAP | FYPGAVTV |
| | 588) | EWVSVIRSK | KDTLMISRTPEVTCVVVDVSDEDGEV | 588) | KLLIYKDDQ | AWKADSSP |
| | | ANSYATYYA | KFNWYVDGVEVHNAKTKPREEQYNST | | RPSGVPDRF | VKAGVETT |
| | | DSVKGRFTI | YRVVSVLTVLHQDWLNGKEYKCKVSN | | SGSQSGTSA | TPSKQSNN |
| | | SRDNSKNTL | KALPRPIEKTISKAKGQPREPQVYTL | | SLAISGLQS | KYAASSYL |
| | | YLQMNSLRA | PPSRDELTKNQVSLTCLVKGFYPSDI | | EDEADYYCG | SLTPEQWK |
| | | EDTAVYYCA | AVEWESNGQPENNYKTTPPVLDSDGS | | TWDDSLNVW | SHRSYSCQ |
| | | RGGTGYFDY | FFLYSKLTVDKSRWQQGNVFSCSVMH | | VFGGGTKLT | VTHEGSTV |
| | | WGQGTLVTV | EALHNHYTQKSLSLSPG (SEQ ID | | VL (SEQ | EKTVAPTE |
| | | SS (SEQ | NO: 587) | | ID NO: | CS (SEQ |
| | | ID NO: | | | 336) | ID NO: |
| | | 181) | | | | 416) |
| TA125 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSRS | EELQANKA |
| | (SEQ ID | TFSGSALSW | QTYICNVNHKPSNTKVDKKVEPKSCD | (SEQ ID | NIGTNRVYW | TLVCLISD |
| | NO: | VRQAPGKGL | KTHTCPPCPAPDLLGDDSVFLFPPKP | NO: | YQQLPGTAP | FYPGAVTV |
| | 588) | EWVSAIFSN | KDTLMISRTPEVTCVVVDVSDEDGEV | 588) | KLLIYKDDQ | AWKADSSP |
| | | GGSAYYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCA | SLTPEQWK |
| | | TAVYYCAKG | AVEWESNGQPENNYKTTPPVLDSDGS | | AWDDSLNGW | SHRSYSCQ |
| | | GYNWNNYLE | FFLYSKLTVDKSRWQQGNVFSCSVMH | | VFGGGTKLT | VTHEGSTV |
| | | YWGQGTLVT | EALHNHYTQKSLSLSPG (SEQ ID | | VL (SEQ | EKTVAPTE |
| | | VSS (SEQ | NO: 587) | | ID NO: | CS (SEQ |
| | | ID NO: | | | 337) | ID NO: |
| | | 182) | | | | 416) |
| TA126 | MGWSCII | EVQLLESGG | ASTKGPSVFPLAPSSKSTSGGTAALG | MGWSCII | QSVLTQPPS | GQPKAAPS |
| | LFLVATA | AFVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSYS | EELQANKA |
| | (SEQ ID | TFSDYYMSW | QTYICNVNHKPSNTKVDKKVEPKSCD | (SEQ ID | NIGNSYVSW | TLVCLISD |
| | NO: | VRQAPGKGL | KTHTCPPCPAPDLLGDDSVFLFPPKP | NO: | YQQLPGTAP | FYPGAVTV |
| | 588) | EWVSVISNS | KDTLMISRTPEVTCVVVDVSDEDGEV | 588) | KLLIYLNSQ | AWKADSSP |
| | | GGSTYYTDS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCA | SLTPEQWK |
| | | TAVYYCTKD | AVEWESNGQPENNYKTTPPVLDSDGS | | AWDDPNVWV | SHRSYSCQ |
| | | IGMTYFDYW | FFLYSKLTVDKSRWQQGNVFSCSVMH | | FGGGTKLTV | VTHEGSTV |
| | | GQGTLVTVS | EALHNHYTQKSLSLSPG (SEQ ID | | L (SEQ ID | EKTVAPTE |
| | | S (SEQ ID | NO: 587) | | NO: 338) | CS (SEQ |
| | | NO: 183) | | | | ID NO: |
| | | | | | | 416) |
| TA127 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSS | EELQANKA |
| | (SEQ ID | TFSNYEMNW | QTYICNVNHKPSNTKVDKKVEPKSCD | (SEQ ID | NIDINYIDW | TLVCLISD |
| | NO: | VRQAPGKGL | KTHTCPPCPAPDLLGDDSVFLFPPKP | NO: | YQQLPGTAP | FYPGAVTV |
| | 588) | EWVGIIGTG | KDTLMISRTPEVTCVVVDVSDEDGEV | 588) | KVLIYNTDQ | AWKADSSP |
| | | GAITYYADS | KFNWYVDGVEVHNAKTKPREEQYNST | | RPSGVPDRF | VKAGVETT |
| | | VKGRFTISR | YRVVSVLIVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCA | SLTPEQWK |
| | | TAIYYCVKY | AVEWESNGQPENNYKTTPPVLDSDGS | | GWDSSLRVW | SHRSYSCQ |
| | | STESYFDYW | FFLYSKLTVDKSRWQQGNVFSCSVMH | | VFGGGTKLT | VTHEGSTV |
| | | GQGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 184) | | | 339) | ID NO: |
| | | | | | | 416) |
| TA128 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | DFSGYDMTW | QTYICNVNHKPSNTKVDKKVEPKSCD | (SEQ ID | NIGRNNYVS | TLVCLISD |
| | NO: | VRQAPGKGL | KTHTCPPCPAPDLLGDDSVFLFPPKP | NO: | WYQQLPGTA | FYPGAVTV |
| | 588) | EWVSSITWN | KDTLMISRTPEVTCVVVDVSDEDGEV | 588) | PKLLIYRDY | AWKADSSP |
| | | SWIDGTKIY | KFNWYVDGVEVHNAKTKPREEQYNST | | QRPSGVPDR | VKAGVETT |
| | | YADSVKGRF | YRVVSVLIVLHQDWLNGKEYKCKVSN | | FSGSKSGTS | TPSKQSNN |
| | | TISRDNSNN | KALPRPIEKTISKAKGQPREPQVYTL | | ASLAISGLQ | KYAASSYL |
| | | TLYLQMNSL | PPSRDELTKNQVSLTCLVKGFYPSDI | | SEDEADYYC | SLTPEQWK |
| | | RDEDTAIYY | AVEWESNGQPENNYKTTPPVLDSDGS | | VAWDDRVNG | SHRSYSCQ |
| | | CAGGSLTVN | FFLYSKLTVDKSRWQQGNVFSCSVMH | | WVFGGGTKL | VTHEGSTV |
| | | YFDYWGQGT | EALHNHYTQKSLSLSPG (SEQ ID | | TVL (SEQ | EKTVAPTE |
| | | LVTVSS | NO: 587) | | ID NO: | CS (SEQ |
| | | (SEQ ID | | | 340) | ID NO: |
| | | NO: 185) | | | | 416) |
| TA129 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFSDYWMTW | QTYICNVNHKPSNTKVDKKVEPKSCD | (SEQ ID | NIRNNYVSW | TLVCLISD |
| | NO: | VRQAPGKGL | KTHTCPPCPAPDLLGDDSVFLFPPKP | NO: | YQQLPGTAP | FYPGAVTV |
| | 588) | EWVSTISDS | KDTLMISRTPEVTCVVVDVSDEDGEV | 588) | KLLIYGKNQ | AWKADSSP |
| | | SNGGRTYYA | KFNWYVDGVEVHNAKTKPREEQYNST | | RPSGVPDRF | VKAGVETT |
| | | DSVKGRFTI | YRVVSVLIVLHQDWLNGKEYKCKVSN | | SGSKSGTSA | TPSKQSNN |
| | | SRDNSKNTL | KALPRPIEKTISKAKGQPREPQVYTL | | SLAISGLQS | KYAASSYL |
| | | YLQMNSLRA | PPSRDELTKNQVSLTCLVKGFYPSDI | | EDEADYYCA | SLTPEQWK |
| | | EDTAVYYCT | AVEWESNGQPENNYKTTPPVLDSDGS | | TWDDSLNGW | SHRSYSCQ |
| | | KFDSWGQGA | FFLYSKLTVDKSRWQQGNVFSCSVMH | | VFGGGTKLT | VTHEGSTV |
| | | LVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 186) | | | 341) | ID NO: |
| | | | | | | 416) |
| TA130 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFSDYWMTW | QTYICNVNHKPSNTKVDKKVEPKSCD | (SEQ ID | NIRNNYVSW | TLVCLISD |
| | NO: | VRQAPGKGL | KTHTCPPCPAPDLLGDDSVFLFPPKP | NO: | YQQLPGTAP | FYPGAVTV |
| | 588) | EWVSTISDS | KDTLMISRTPEVTCVVVDVSDEDGEV | 588) | KLLIYGKNQ | AWKADSSP |
| | | SNGGRTYYA | KFNWYVDGVEVHNAKTKPREEQYNST | | RPSGVPDRF | VKAGVETT |
| | | DSVKGRFTI | YRVVSVLTVLHQDWLNGKEYKCKVSN | | SGSKSGTSA | TPSKQSNN |
| | | SRDNSKNTL | KALPRPIEKTISKAKGQPREPQVYTL | | SLAISGLQS | KYAASSYL |
| | | YLQMNSLRA | PPSRDELTKNQVSLTCLVKGFYPSDI | | EDEADYYCS | SLTPEQWK |
| | | EDTAVYYCT | AVEWESNGQPENNYKTTPPVLDSDGS | | TWDDSLNGW | SHRSYSCQ |
| | | KFDSWGQGA | FFLYSKLTVDKSRWQQGNVFSCSVMH | | VFGGGTKLT | VTHEGSTV |
| | | LVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 186) | | | 342) | ID NO: |
| | | | | | | 416) |
| TA131 | MGWSCII | EVQLLESGG | ASTKGPSVFPLAPSSKSTSGGTAALG | MGWSCII | QSVLTQPPS | GQPKAAPS |
| | LFLVATA | ALVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSYS | EELQANKA |
| | (SEQ ID | TFSDYYMSW | QTYICNVNHKPSNTKVDKKVEPKSCD | (SEQ ID | NIGNSYVSW | TLVCLISD |
| | NO: | VRQAPGKGL | KTHTCPPCPAPDLLGDDSVFLFPPKP | NO: | YQQLPGTAP | FYPGAVTV |
| | 588) | EWVSVISNS | KDTLMISRTPEVTCVVVDVSDEDGEV | 588) | KLLIYLNSQ | AWKADSSP |
| | | GGSTYYADS | KFNWYVDGVEVHNAKTKPREEQYNST | | RPSGVPDRF | VKAGVETT |
| | | VKGRFTISR | YRVVSVLIVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCA | SLTPEQWK |
| | | TAVYYCTKD | AVEWESNGQPENNYKTTPPVLDSDGS | | AWDDLNVWV | SHRSYSCQ |
| | | IGMTYFDYW | FFLYSKLTVDKSRWQQGNVFSCSVMH | | FGGGTKLTV | VTHEGSTV |
| | | GQGTLVTVS | EALHNHYTQKSLSLSPG (SEQ ID | | L (SEQ ID | EKTVAPTE |
| | | S (SEQ ID | NO: 587) | | NO: 343) | CS (SEQ |
| | | NO: 180) | | | | ID NO: |
| | | | | | | 416) |
| TA132 | MGWSCII | EVQLLESGG | ASTKGPSVFPLAPSSKSTSGGTAALG | MGWSCII | QSVLTQPPS | GQPKAAPS |
| | LFLVATA | AFVQPGGSL | CLVKDYFPEPVTVSWNSGALTSGVHT | LFLVATA | ASGAPGQRV | VTLFPPSS |
| | TGVHS | RLSCAASGF | FPAVLQSSGLYSLSSVVTVPSSSLGT | TGVHS | TISCSGSYS | EELQANKA |
| | (SEQ ID | TFSDYYMSW | QTYICNVNHKPSNTKVDKKVEPKSCD | (SEQ ID | NIGNSYVSW | TLVCLISD |
| | NO: | VRQAPGKGL | KTHTCPPCPAPDLLGDDSVFLFPPKP | NO: | YQQLPGTAP | FYPGAVTV |
| | 588) | EWVSVISNS | KDTLMISRTPEVTCVVVDVSDEDGEV | 588) | KLLIYLNSQ | AWKADSSP |
| | | GGSTYYTDS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QINSLRAED | PPSRDELTKNQVSLTCLVKGFYPSDI | | EDEADYYCA | SLTPEQWK |
| | | TAVYYCTKD | AVEWESNGQPENNYKTTPPVLDSDGS | | AWDDLNVWV | SHRSYSCQ |
| | | IGMTYFDYW | FFLYSKLTVDKSRWQQGNVFSCSVMH | | FGGGTKLTV | VTHEGSTV |
| | | GQGTLVTVS | EALHNHYTQKSLSLSPG (SEQ ID | | L (SEQ ID | EKTVAPTE |
| | | S (SEQ ID | NO: 587) | | NO: 344) | CS (SEQ |
| | | NO: 187) | | | | ID NO: |
| | | | | | | 416) |
| TA133 | MGWSCII | EVQLLESGG | ASTKGPSVFPLAPSSKSTSGGTAALG | MGWSCII | QSVLTQPPS | GQPKAAPS |
| | LFLVATA | AFVQPGGSL | CLVKDYFPEPVTVSWNSGALTSGVHT | LFLVATA | ASGAPGQRV | VTLFPPSS |
| | TGVHS | RLSCAASGF | FPAVLQSSGLYSLSSVVTVPSSSLGT | TGVHS | TISCSGSYS | EELQANKA |
| | (SEQ ID | TFSDYYMSW | QTYICNVNHKPSNTKVDKKVEPKSCD | (SEQ ID | NIGNSYVSW | TLVCLISD |
| | NO: | VRQAPGKGL | KTHTCPPCPAPDLLGDDSVFLFPPKP | NO: | YQQLPGTAP | FYPGAVTV |
| | 588) | EWVSVISNS | KDTLMISRTPEVTCVVVDVSDEDGEV | 588) | KLLIYLNSQ | AWKADSSP |
| | | GGSTYYTDS | KFNWYVDGVEVHNAKTKPREEQYNST | | RPSGVPDRF | VKAGVETT |
| | | VKGRFTISR | YRVVSVLIVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCA | SLTPEQWK |
| | | TAVYYCTKD | AVEWESNGQPENNYKTTPPVLDSDGS | | AWDDLNVWV | SHRSYSCQ |
| | | IGMTYFDYW | FFLYSKLTVDKSRWQQGNVFSCSVMH | | FGGGTKLTV | VTHEGSTV |
| | | GQGTLVTVS | EALHNHYTQKSLSLSPG (SEQ ID | | L (SEQ ID | EKTVAPTE |
| | | S (SEQ ID | NO: 587) | | NO: 344) | CS (SEQ |
| | | NO: 183) | | | | ID NO: |
| | | | | | | 416) |
| TA134 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | DFSGYDMTW | QTYICNVNHKPSNTKVDKKVEPKSCD | (SEQ ID | NIGRNNYVS | TLVCLISD |
| | NO: | VRQAPGKGL | KTHTCPPCPAPDLLGDDSVFLFPPKP | NO: | WYQQLPGTA | FYPGAVTV |
| | 588) | EWVSSITWN | KDTLMISRTPEVTCVVVDVSDEDGEV | 588) | PKLLIYRDY | AWKADSSP |
| | | SWIDGTKIY | KFNWYVDGVEVHNAKTKPREEQYNST | | QRPSGAPDR | VKAGVETT |
| | | YADSVKGRF | YRVVSVLTVLHQDWLNGKEYKCKVSN | | FSGSKSGTS | TPSKQSNN |
| | | TISRDNSNN | KALPRPIEKTISKAKGQPREPQVYTL | | ASLAISGLQ | KYAASSYL |
| | | TLYLQMNSL | PPSRDELTKNQVSLTCLVKGFYPSDI | | SEDEADYYC | SLTPEQWK |
| | | RDEDTAIYY | AVEWESNGQPENNYKTTPPVLDSDGS | | VAWDDRVNG | SHRSYSCQ |
| | | CAGGSLTVN | FFLYSKLTVDKSRWQQGNVFSCSVMH | | WVFGGGTKL | VTHEGSTV |
| | | YFDYWGQGT | EALHNHYTQKSLSLSPG (SEQ ID | | TVL (SEQ | EKTVAPTE |
| | | LVTVSS | NO: 587) | | ID NO: | CS (SEQ |
| | | (SEQ ID | | | 345) | ID NO: |
| | | NO: 185) | | | | 416) |
| TA135 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSRS | EELQANKA |
| | (SEQ ID | TFSGSALSW | QTYICNVNHKPSNTKVDKKVEPKSCD | (SEQ ID | NIGTNRVYW | TLVCLISD |
| | NO: | VRQAPGKGL | KTHTCPPCPAPDLLGDDSVFLFPPKP | NO: | YQQLPGTAP | FYPGAVTV |
| | 588) | EWVSAIFSN | KDTLMISRTPEVTCVVVDVSDEDGEV | 588) | KLLIYKDDQ | AWKADSSP |
| | | GGSAYYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCA | SLTPEQWK |
| | | TAVYYCAEG | AVEWESNGQPENNYKTTPPVLDSDGS | | SWDDSLNGW | SHRSYSCQ |
| | | GYNWNNYLE | FFLYSKLTVDKSRWQQGNVFSCSVMH | | VFGGGTKLT | VTHEGSTV |
| | | YWGQGTLVT | EALHNHYTQKSLSLSPG (SEQ ID | | VL (SEQ | EKTVAPTE |
| | | VSS (SEQ | NO: 587) | | ID NO: | CS (SEQ |
| | | ID NO: | | | 346) | ID NO: |
| | | 188) | | | | 416) |
| TA136 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSF | EELQANKA |
| | (SEQ ID | TFSDYDMAW | QTYICNVNHKPSNTKVDKKVEPKSCD | (SEQ ID | NIGTRYVYW | TLVCLISD |
| | NO: | VRQAPGKGL | KTHTCPPCPAPDLLGDDSVFLFPPKP | NO: | YQQLPGTAP | FYPGAVTV |
| | 588) | EWVSTITNT | KDTLMISRTPEVTCVVVDVSDEDGEV | 588) | KLLIYRNSQ | AWKADSSP |
| | | GSHIYYADS | KFNWYVDGVEVHNAKTKPREEQYNST | | RPSGVPDRF | VKAGVETT |
| | | VKGRSTISR | YRVVSVLTVLHQDWLNGKEYKCKVSN | | SGSKSGTSA | TPSKQSNN |
| | | DNSENTLYL | KALPRPIEKTISKAKGQPREPQVYTL | | SLAISGLQA | KYAASSYL |
| | | QMNSLRAED | PPSRDELTKNQVSLTCLVKGFYPSDI | | EDEADYYCA | SLTPEQWK |
| | | TAVYYCVKE | AVEWESNGQPENNYKTTPPVLDSDGS | | AWDDSLNGW | SHRSYSCQ |
| | | GTITIFDYW | FFLYSKLTVDKSRWQQGNVFSCSVMH | | VFGGGTKLT | VTHEGSTV |
| | | GQGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 189) | | | 347) | ID NO: |
| | | | | | | 416) |
| TA137 | MGWSCII | EVQLLESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSRS | EELQANKA |
| | (SEQ ID | TFSGSALSW | QTYICNVNHKPSNTKVDKKVEPKSCD | (SEQ ID | NIGTNRVYW | TLVCLISD |
| | NO: | VRQAPGKGL | KTHTCPPCPAPDLLGDDSVFLFPPKP | NO: | YQQLPGTAP | FYPGAVTV |
| | 588) | EWVSAIFSN | KDTLMISRTPEVTCVVVDVSDEDGEV | 588) | KLLIYKDDQ | AWKADSSP |
| | | GGSAYYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCA | SLTPEQWK |
| | | TAVYYCAKG | AVEWESNGQPENNYKTTPPVLDSDGS | | SWDDSLNGW | SHRSYSCQ |
| | | GYNWNNYLE | FFLYSKLTVDKSRWQQGNVFSCSVMH | | VFGGGTKLT | VTHEGSTV |
| | | YWGQGTLVT | EALHNHYTQKSLSLSPG (SEQ ID | | VL (SEQ | EKTVAPTE |
| | | VSS (SEQ | NO: 587) | | ID NO: | CS (SEQ |
| | | ID NO: | | | 346) | ID NO: |
| | | 182) | | | | 416) |
| TA138 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | GSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMSSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WAYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 190) | | | | ID NO: |
| | | | | | | 415) |
| TA139 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | PSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLGAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYSDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 191) | | | | ID NO: |
| | | | | | | 415) |
| TA140 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | PSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWDQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 192) | | | | ID NO: |
| | | | | | | 415) |
| TA141 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | IFSGYDMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAIIWN | KDTLMISRTPEVTCVVVDVSDEDGEV | 588) | LLIYGASTR | WKVDNALQ |
| | | SNTIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTVYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCARG | AVEWESNGQPENNYKTTPPVLDSDGS | | YNNWPPWTF | KHKVYACE |
| | | YNLKNYFDY | FFLYSKLTVDKSRWQQGNVFSCSVMH | | GQGTKVEIK | VTHQGLSS |
| | | WGQGTLVTV | EALHNHYTQKSLSLSPG (SEQ ID | | (SEQ ID | PVTKSFNR |
| | | SS (SEQ | NO: 587) | | NO: 323) | GEC (SEQ |
| | | ID NO: | | | | ID NO: |
| | | 193) | | | | 415) |
| TA142 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | GSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSENTLYL | KALPRPIEKTISKAKGQPREPQVYTL | | LTISSLQSE | TYSLSSTL |
| | | QMSSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WAYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 194) | | | | ID NO: |
| | | | | | | 415) |
| TA143 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | IFSGYDMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAIIWN | KDTLMISRTPEVTCVVVDVSDEDGEV | 588) | LLIYGASTR | WKVDNALQ |
| | | SNTIYYADS | KFNWYVDGVEVHNAKTKPREEQYNST | | ATGIPARFS | SGNSQESV |
| | | VKGRFTISR | YRVVSVLIVLHQDWLNGKEYKCKVSN | | GSGSGTEFT | TEQDSKDS |
| | | DNSKNTVYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCVRG | AVEWESNGQPENNYKTTPPVLDSDGS | | YNNWPPWTF | KHKVYACE |
| | | YNLKNYFDY | FFLYSKLTVDKSRWQQGNVFSCSVMH | | GQGTKVEIK | VTHQGLSS |
| | | WGQGTLVTV | EALHNHYTQKSLSLSPG (SEQ ID | | (SEQ ID | PVTKSFNR |
| | | SS (SEQ | NO: 587) | | NO: 323) | GEC (SEQ |
| | | ID NO: | | | | ID NO: |
| | | 195) | | | | 415) |
| TA144 | MGWSCII | EVQLLESGG | ASTKGPSVFPLAPSSKSTSGGTAALG | MGWSCII | EIVMTQSPA | RTVAAPSV |
| | LFLVATA | GLVQPGGVL | CLVKDYFPEPVTVSWNSGALTSGVHT | LFLVATA | TLSVSPGER | FIFPPSDE |
| | TGVHS | RLSCTASGF | FPAVLQSSGLYSLSSVVTVPSSSLGT | TGVHS | ATLSCRASQ | QLKSGTAS |
| | (SEQ ID | TFDDYGMHW | QTYICNVNHKPSNTKVDKKVEPKSCD | (SEQ ID | SVSSNLAWY | VVCLLNNF |
| | NO: | VRQAPEKGL | KTHTCPPCPAPDLLGDDSVFLFPPKP | NO: | QQKPGQAPR | YPREAKVQ |
| | 588) | EWVGAINWN | KDTLMISRTPEVTCVVVDVSDEDGEV | 588) | LLIYGASTR | WKVDNALQ |
| | | GNVTHYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | RMNSLRAED | PPSRDELTKNQVSLTCLVKGFYPSDI | | DFAVYYCQQ | TLSKADYE |
| | | TAVYYCAKN | AVEWESNGQPENNYKTTPPVLDSDGS | | YNNWPPWTF | KHKVYACE |
| | | SGSEKRNYY | FFLYSKLTVDKSRWQQGNVFSCSVMH | | GQGTKVEIK | VTHQGLSS |
| | | FGYWGQGTL | EALHNHYTQKSLSLSPG (SEQ ID | | (SEQ ID | PVTKSFNR |
| | | VTVSS | NO: 587) | | NO: 323) | GEC (SEQ |
| | | (SEQ ID | | | | ID NO: |
| | | NO: 196) | | | | 415) |
| TA145 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDHYMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTISSG | KDTLMISRTPEVTCVVVDVSDEDGEV | 588) | LLIYGASTR | WKVDNALQ |
| | | GNYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DSSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCARI | AVEWESNGQPENNYKTTPPVLDSDGS | | YNNWPPWTF | KHKVYACE |
| | | LKNGKYIYY | FFLYSKLTVDKSRWQQGNVFSCSVMH | | GQGTKVEIK | VTHQGLSS |
| | | FDYWGQGTL | EALHNHYTQKSLSLSPG (SEQ ID | | (SEQ ID | PVTKSFNR |
| | | VTVSS | NO: 587) | | NO: 323) | GEC (SEQ |
| | | (SEQ ID | | | | ID NO: |
| | | NO: 156) | | | | 415) |
| TA146 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFGDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTIYSG | KDTLMISRTPEVTCVVVDVSDEDGEV | 588) | LLIYGASTR | WKVDNALQ |
| | | VATIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCARG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 197) | | | | ID NO: |
| | | | | | | 415) |
| TA147 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCTASGF | FPAVLQSSGLYSLSSVVTVPSSSLGT | TGVHS | ATLSCRASQ | QLKSGTAS |
| | (SEQ ID | TFDDYGMHW | QTYICNVNHKPSNTKVDKKVEPKSCD | (SEQ ID | SVSSNLAWY | VVCLLNNF |
| | NO: | VRQAPEKGL | KTHTCPPCPAPDLLGDDSVFLFPPKP | NO: | QQKPGQAPR | YPREAKVQ |
| | 588) | EWVGAINWN | KDTLMISRTPEVTCVVVDVSDEDGEV | 588) | LLIYGASTR | WKVDNALQ |
| | | GNVTHYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | RMNSLRAED | PPSRDELTKNQVSLTCLVKGFYPSDI | | DFAVYYCQQ | TLSKADYE |
| | | TAVYYCAKN | AVEWESNGQPENNYKTTPPVLDSDGS | | YNNWPPWTF | KHKVYACE |
| | | SGSEKRNYY | FFLYSKLTVDKSRWQQGNVFSCSVMH | | GQGTKVEIK | VTHQGLSS |
| | | FGYWGQGTL | EALHNHYTQKSLSLSPG (SEQ ID | | (SEQ ID | PVTKSFNR |
| | | VTVSS | NO: 587) | | NO: 323) | GEC (SEQ |
| | | (SEQ ID | | | | ID NO: |
| | | NO: 198) | | | | 415) |
| TA148 | MGWSCII | EVQLLESGG | ASTKGPSVFPLAPSSKSTSGGTAALG | MGWSCII | EIVMTQSPA | RTVAAPSV |
| | LFLVATA | GLVQPGGPL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYTG | KDTLMISRTPEVTCVVVDVSDEDGEV | 588) | LLIYGASTR | WKVDNALQ |
| | | GSSYIYYAD | KFNWYVDGVEVHNAKTKPREEQYNST | | ATGIPARFS | SGNSQESV |
| | | SVKGRFTIS | YRVVSVLTVLHQDWLNGKEYKCKVSN | | GSGSGTEFT | TEQDSKDS |
| | | RDNSKNTLY | KALPRPIEKTISKAKGQPREPQVYTL | | LTISSLQSE | TYSLSSTL |
| | | LQMNSLRAE | PPSRDELTKNQVSLTCLVKGFYPSDI | | DFAVYYCQQ | TLSKADYE |
| | | DTAVYYCTR | AVEWESNGQPENNYKTTPPVLDSDGS | | YNNWPPWTF | KHKVYACE |
| | | GWTYFDYWG | FFLYSKLTVDKSRWQQGNVFSCSVMH | | GQGTKVEIK | VTHQGLSS |
| | | QGTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 199) | | | | ID NO: |
| | | | | | | 415) |
| TA149 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCTASGF | FPAVLQSSGLYSLSSVVTVPSSSLGT | TGVHS | ATLSCRASQ | QLKSGTAS |
| | (SEQ ID | TFDDYGMHW | QTYICNVNHKPSNTKVDKKVEPKSCD | (SEQ ID | SVSSNLAWY | VVCLLNNF |
| | NO: | VRQAPEKGL | KTHTCPPCPAPDLLGDDSVFLFPPKP | NO: | QQKPGQAPR | YPREAKVQ |
| | 588) | EWVGAVNWN | KDTLMISRTPEVTCVVVDVSDEDGEV | 588) | LLIYGASTR | WKVDNALQ |
| | | GNVTHYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | RMNSLRAED | PPSRDELTKNQVSLTCLVKGFYPSDI | | DFAVYYCQQ | TLSKADYE |
| | | TAVYYCAKN | AVEWESNGQPENNYKTTPPVLDSDGS | | YNNWPPWTF | KHKVYACE |
| | | SGSEKRNYY | FFLYSKLTVDKSRWQQGNVFSCSVMH | | GQGTKVEIK | VTHQGLSS |
| | | FGYWGQGTL | EALHNHYTQKSLSLSPG (SEQ ID | | (SEQ ID | PVTKSFNR |
| | | VTVSS | NO: 587) | | NO: 323) | GEC (SEQ |
| | | (SEQ ID | | | | ID NO: |
| | | NO: 200) | | | | 415) |
| TA150 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFDDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAIYSG | KDTLMISRTPEVTCVVVDVSDEDGEV | 588) | LLIYGASTR | WKVDNALQ |
| | | SYIYYADSV | KFNWYVDGVEVHNAKTKPREEQYNST | | ATGIPARFS | SGNSQESV |
| | | KGRFTISRD | YRVVSVLTVLHQDWLNGKEYKCKVSN | | GSGSGTEFT | TEQDSKDS |
| | | NSKNTLYLQ | KALPRPIEKTISKAKGQPREPQVYTL | | LTISSLQSE | TYSLSSTL |
| | | MNSLRAEDT | PPSRDELTKNQVSLTCLVKGFYPSDI | | DFAVYYCQQ | TLSKADYE |
| | | AVYYCARGW | AVEWESNGQPENNYKTTPPVLDSDGS | | YNNWPPWTF | KHKVYACE |
| | | SYFDYWGQG | FFLYSKLTVDKSRWQQGNVFSCSVMH | | GQGTKVEIK | VTHQGLSS |
| | | TLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 201) | | | | ID NO: |
| | | | | | | 415) |
| TA151 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | TTYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSENTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYRGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 202) | | | | ID NO: |
| | | | | | | 415) |
| TA152 | MGWSCII | EVQLLESGG | ASTKGPSVFPLAPSSKSTSGGTAALG | MGWSCII | EIVMTQSPA | RTVAAPSV |
| | LFLVATA | GLIQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | SSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 203) | | | | ID NO: |
| | | | | | | 415) |
| TA153 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | VSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 204) | | | | ID NO: |
| | | | | | | 415) |
| TA154 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSGYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | SSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DKSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 205) | | | | ID NO: |
| | | | | | | 415) |
| TA155 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | PSYIYYADS | KFNWYVDGVEVHNAKTKPREEQYNST | | ATGIPARFS | SGNSQESV |
| | | VKGRFTISR | YRVVSVLIVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYLGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 206) | | | | ID NO: |
| | | | | | | 415) |
| TA156 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | TTYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSENTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 130) | | | | ID NO: |
| | | | | | | 415) |
| TA157 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYTG | KDTLMISRTPEVTCVVVDVSDEDGEV | 588) | LLIYGASTR | WKVDNALQ |
| | | GSSYIYYAD | KFNWYVDGVEVHNAKTKPREEQYNST | | ATGIPARFS | SGNSQESV |
| | | SVKGRFTIS | YRVVSVLTVLHQDWLNGKEYKCKVSN | | GSGSGTEFT | TEQDSKDS |
| | | RDNSKNTLY | KALPRPIEKTISKAKGQPREPQVYTL | | LTISSLQSE | TYSLSSTL |
| | | LQMNSLRAE | PPSRDELTKNQVSLTCLVKGFYPSDI | | DFAVYYCQQ | TLSKADYE |
| | | DTAVYYCTR | AVEWESNGQPENNYKTTPPVLDSDGS | | YNNWPPWTF | KHKVYACE |
| | | GWTYFDYWG | FFLYSKLTVDKSRWQQGNVFSCSVMH | | GQGTKVEIK | VTHQGLSS |
| | | QGTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 207) | | | | ID NO: |
| | | | | | | 415) |
| TA158 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | ATYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 208) | | | | ID NO: |
| | | | | | | 415) |
| TA159 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYTG | KDTLMISRTPEVTCVVVDVSDEDGEV | 588) | LLIYGASTR | WKVDNALQ |
| | | GSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 209) | | | | ID NO: |
| | | | | | | 415) |
| TA160 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | PSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 135) | | | | ID NO: |
| | | | | | | 415) |
| TA161 | MGWSCII | EVQLLESGG | ASTKGPSVFPLAPSSKSTSGGTAALG | MGWSCII | EIVMTQSPA | RTVAAPSV |
| | LFLVATA | GLVQPGESL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | PSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYHCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 210) | | | | ID NO: |
| | | | | | | 415) |
| TA162 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | PTYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 211) | | | | ID NO: |
| | | | | | | 415) |
| TA163 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | IFSGYDMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAIIWN | KDTLMISRTPEVTCVVVDVSDEDGEV | 588) | LLIYGASTR | WKVDNALQ |
| | | SNTIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTVYL | KALPRPIEKTISKAKGQPREPQVYTL | | LTISSLQSE | TYSLSSTL |
| | | RMNSLRAED | PPSRDELTKNQVSLTCLVKGFYPSDI | | DFAVYYCQQ | TLSKADYE |
| | | TAVYYCARG | AVEWESNGQPENNYKTTPPVLDSDGS | | YNNWPPWTF | KHKVYACE |
| | | YNLKNYFDY | FFLYSKLTVDKSRWQQGNVFSCSVMH | | GQGTKVEIK | VTHQGLSS |
| | | WGQGTLVTV | EALHNHYTQKSLSLSPG (SEQ ID | | (SEQ ID | PVTKSFNR |
| | | SS (SEQ | NO: 587) | | NO: 323) | GEC (SEQ |
| | | ID NO: | | | | ID NO: |
| | | 212) | | | | 415) |
| TA164 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | VSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 204) | | | | ID NO: |
| | | | | | | 415) |
| TA165 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAAPGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | GSYIYYADS | KFNWYVDGVEVHNAKTKPREEQYNST | | ATGIPARFS | SGNSQESV |
| | | VKGRFTISR | YRVVSVLIVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMSSLRAED | PPSRDELTKNQVSLTCLVKGFYPSDI | | DEAVYYCQQ | TLSKADYE |
| | | TAVYYCTRG | AVEWESNGQPENNYKTTPPVLDSDGS | | YNNWPPWTF | KHKVYACE |
| | | WAYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 213) | | | | ID NO: |
| | | | | | | 415) |
| TA166 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | GPYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 214) | | | | ID NO: |
| | | | | | | 415) |
| TA167 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFDDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTIYSG | KDTLMISRTPEVTCVVVDVSDEDGEV | 588) | LLIYGASTR | WKVDNALQ |
| | | VATIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCARG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 215) | | | | ID NO: |
| | | | | | | 415) |
| TA168 | MGWSCII | EVQLLEFGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFDDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTIYSG | KDTLMISRTPEVTCVVVDVSDEDGEV | 588) | LLIYGASTR | WKVDNALQ |
| | | VATIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCARG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 216) | | | | ID NO: |
| | | | | | | 415) |
| TA169 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSTIYSV | KDTLMISRTPEVTCVVVDVSDEDGEV | 588) | LLIYGASTR | WKVDNALQ |
| | | GTIYYADSV | KFNWYVDGVEVHNAKTKPREEQYNST | | ATGIPARFS | SGNSQESV |
| | | KGRFTISRD | YRVVSVLTVLHQDWLNGKEYKCKVSN | | GSGSGTEFT | TEQDSKDS |
| | | NSKNTLYLQ | KALPRPIEKTISKAKGQPREPQVYTL | | LTISSLQSE | TYSLSSTL |
| | | MDSLRAEDT | PPSRDELTKNQVSLTCLVKGFYPSDI | | DFAVYYCQQ | TLSKADYE |
| | | AVYYCARGW | AVEWESNGQPENNYKTTPPVLDSDGS | | YNNWPPWTF | KHKVYACE |
| | | TYFDYWGQG | FFLYSKLTVDKSRWQQGNVFSCSVMH | | GQGTKVEIK | VTHQGLSS |
| | | TLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 217) | | | | ID NO: |
| | | | | | | 415) |
| TA170 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | IFSGYDMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAIGWK | KDTLMISRTPEVTCVVVDVSDEDGEV | 588) | LLIYGASTR | WKVDNALQ |
| | | SGSIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCAKG | AVEWESNGQPENNYKTTPPVLDSDGS | | YNNWPPWTF | KHKVYACE |
| | | YNLKNYFDY | FFLYSKLTVDKSRWQQGNVFSCSVMH | | GQGTKVEIK | VTHQGLSS |
| | | WGQGTLVTV | EALHNHYTQKSLSLSPG (SEQ ID | | (SEQ ID | PVTKSFNR |
| | | SS (SEQ | NO: 587) | | NO: 323) | GEC (SEQ |
| | | ID NO: | | | | ID NO: |
| | | 218) | | | | 415) |
| TA171 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLPCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | VSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 219) | | | | ID NO: |
| | | | | | | 415) |
| TA172 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | PSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSPRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 220) | | | | ID NO: |
| | | | | | | 415) |
| TA173 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | IFSGYDMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAISWT | KDTLMISRTPEVTCVVVDVSDEDGEV | 588) | LLIYGASTR | WKVDNALQ |
| | | SDSIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNFENTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCAKG | AVEWESNGQPENNYKTTPPVLDSDGS | | YNNWPPWTF | KHKVYACE |
| | | YNRNNYFDY | FFLYSKLTVDKSRWQQGNVFSCSVMH | | GQGTKVEIK | VTHQGLSS |
| | | WGQGTLVTV | EALHNHYTQKSLSLSPG (SEQ ID | | (SEQ ID | PVTKSFNR |
| | | SS (SEQ | NO: 587) | | NO: 323) | GEC (SEQ |
| | | ID NO: | | | | ID NO: |
| | | 221) | | | | 415) |
| TA174 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSFIYSG | KDTLMISRTPEVTCVVVDVSDEDGEV | 588) | LLIYGASTR | WKVDNALQ |
| | | PSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 222) | | | | ID NO: |
| | | | | | | 415) |
| TA175 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQASGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | GSYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMSSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WAYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 223) | | | | ID NO: |
| | | | | | | 415) |
| TA176 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | ASYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 224) | | | | ID NO: |
| | | | | | | 415) |
| TA177 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSAYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSSIYSG | KDTLMISRTPEVTCVVVDVSDEDGEV | 588) | LLIYGASTR | WKVDNALQ |
| | | TTYIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSENTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCTRG | AVEWESNGQPENNYKTTPPVLDSDGS | | YNNWPPWTF | KHKVYACE |
| | | WTYFDYWGQ | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 225) | | | | ID NO: |
| | | | | | | 415) |
| TA178 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFNDYWMSW | QTYICNVNHKPSNTKVDKKVEPKSCD | (SEQ ID | SVSSNLAWY | VVCLLNNF |
| | NO: | VRQAPGKEL | KTHTCPPCPAPDLLGDDSVFLFPPKP | NO: | QQKPGQAPR | YPREAKVQ |
| | 588) | EWVSAIYSG | KDTLMISRTPEVTCVVVDVSDEDGEV | 588) | LLIYGASTR | WKVDNALQ |
| | | GSSSYIYYA | KFNWYVDGVEVHNAKTKPREEQYNST | | ATGIPARFS | SGNSQESV |
| | | DSVKGRFTI | YRVVSVLTVLHQDWLNGKEYKCKVSN | | GSGSGTEFT | TEQDSKDS |
| | | SRDNSKNTL | KALPRPIEKTISKAKGQPREPQVYTL | | LTISSLQSE | TYSLSSTL |
| | | YLQMNSLRA | PPSRDELTKNQVSLTCLVKGFYPSDI | | DFAVYYCQQ | TLSKADYE |
| | | EDTAVYYCA | AVEWESNGQPENNYKTTPPVLDSDGS | | YNNWPPWTF | KHKVYACE |
| | | RGWSYFDYW | FFLYSKLTVDKSRWQQGNVFSCSVMH | | GQGTKVEIK | VTHQGLSS |
| | | GQGTLVTVS | EALHNHYTQKSLSLSPG (SEQ ID | | (SEQ ID | PVTKSFNR |
| | | S (SEQ ID | NO: 587) | | NO: 323) | GEC (SEQ |
| | | NO: 226) | | | | ID NO: |
| | | | | | | 415) |
| TA179 | MGWSCII | EVQLLESGG | ASTKGPSVFPLAPSSKSTSGGTAALG | MGWSCII | EIVMTQSPA | RTVAAPSV |
| | LFLVATA | GSVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | AFSSYWMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAMTGT | KDTLMISRTPEVTCVVVDVSDEDGEV | 588) | LLIYGASTR | WKVDNALQ |
| | | SYIYYADSV | KFNWYVDGVEVHNAKTKPREEQYNST | | ATGIPARFS | SGNSQESV |
| | | KGRFTISRD | YRVVSVLTVLHQDWLNGKEYKCKVSN | | GSGSGTEFT | TEQDSKDS |
| | | NSKNTLYLQ | KALPRPIEKTISKAKGQPREPQVYTL | | LTISSLQSE | TYSLSSTL |
| | | MNSLRAEDT | PPSRDELTKNQVSLTCLVKGFYPSDI | | DFAVYYCQQ | TLSKADYE |
| | | AVYYCARGW | AVEWESNGQPENNYKTTPPVLDSDGS | | YNNWPPWTF | KHKVYACE |
| | | AYLDYWGQG | FFLYSKLTVDKSRWQQGNVFSCSVMH | | GQGTKVEIK | VTHQGLSS |
| | | TLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 227) | | | | ID NO: |
| | | | | | | 415) |
| TA180 | MGWSCII | EVQLLESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | IFSGYDMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSAISWT | KDTLMISRTPEVTCVVVDVSDEDGEV | 588) | LLIYGASTR | WKVDNALQ |
| | | SGSIYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSLNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCAKG | AVEWESNGQPENNYKTTPPVLDSDGS | | YNNWPPWTF | KHKVYACE |
| | | YNRNNYFDY | FFLYSKLTVDKSRWQQGNVFSCSVMH | | GQGTKVEIK | VTHQGLSS |
| | | WGQGTLVTV | EALHNHYTQKSLSLSPG (SEQ ID | | (SEQ ID | PVTKSFNR |
| | | SS (SEQ | NO: 587) | | NO: 323) | GEC (SEQ |
| | | ID NO: | | | | ID NO: |
| | | 228) | | | | 415) |
| TA181 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | NFSSYDMNW | QTYICNVNHKPSNTKVDKKVEPKSCD | (SEQ ID | NIGTRYVYW | TLVCLISD |
| | NO: | VRQAPGEGL | KTHTCPPCPAPDLLGDDSVFLFPPKP | NO: | YQQLPGTAP | FYPGAVTV |
| | 588) | QWVATITAA | KDTLMISRTPEVTCVVVDVSDEDGEV | 588) | KLLIYRNFE | AWKADSSP |
| | | GDITYYADS | KFNWYVDGVEVHNAKTKPREEQYNST | | RPSGVPDRF | VKAGVETT |
| | | VRGRFAISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | GLSSLRAED | PPSRDELTKNQVSLTCLVKGFYPSDI | | EDEADYYCA | SLTPEQWK |
| | | TAIYYCGRE | AVEWESNGQPENNYKTTPPVLDSDGS | | AWDDRVNGW | SHRSYSCQ |
| | | PWNSFSDYW | FFLYSKLTVDKSRWQQGNVFSCSVMH | | LFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 158) | | | 348) | ID NO: |
| | | | | | | 416) |
| TA182 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSS | EELQANKA |
| | (SEQ ID | TFSNFYMNW | QTYICNVNHKPSNTKVDKKVEPKSCD | (SEQ ID | NIGTNTVDW | TLVCLISD |
| | NO: | VRQAPGEGL | KTHTCPPCPAPDLLGDDSVFLFPPKP | NO: | YQQLPGTAP | FYPGAVTV |
| | 588) | EWVSTISGI | KDTLMISRTPEVTCVVVDVSDEDGEV | 588) | KLLIYDNFE | AWKADSSP |
| | | DDTTWYADS | KFNWYVDGVEVHNAKTKPREEQYNST | | RPSGVPDRF | VKAGVETT |
| | | VKGRFAISR | YRVVSVLTVLHQDWLNGKEYKCKVSN | | SGSKSGISA | TPSKQSNN |
| | | DNSRNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCG | SLTPEQWK |
| | | TATYYCAKG | AVEWESNGQPENNYKTTPPVLDSDGS | | TWDDSLNAW | SHRSYSCQ |
| | | TDFLDYWGL | FFLYSKLTVDKSRWQQGNVFSCSVMH | | VFGGGTKLT | VTHEGSTV |
| | | GTLVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 229) | | | 349) | ID NO: |
| | | | | | | 416) |
| TA183 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSNS | EELQANKA |
| | (SEQ ID | TFSDYDMAW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYKNFE | AWKADSSP |
| | | DDSTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGPKSGTSA | TPSKQSNN |
| | | DNSNNTLYL | KALPRPIEKTISKAKGQPREPQVYTL | | SLAISGLRS | KYAASSYL |
| | | RLFSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 230) | | | 350) | ID NO: |
| | | | | | | 416) |
| TA184 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFRNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DDTTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFSLRAED | PPSRDELTKNQVSLTCLVKGFYPSDI | | EDEADYYCG | SLTPEQWK |
| | | TAIYYCARE | AVEWESNGQPENNYKTTPPVLDSDGS | | TWDDSLNSW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 160) | | | 351) | ID NO: |
| | | | | | | 416) |
| TA185 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCIASGE | FPAVLQSSGLYSLSSVVTVPSSSLGT | TGVHS | TISCSGSTF | EELQANKA |
| | (SEQ ID | TFTTYDMSW | QTYICNVNHKPSNTKVDKKVEPKSCD | (SEQ ID | NIGTNYVSW | TLVCLISD |
| | NO: | VRQAPGEGL | KTHTCPPCPAPDLLGDDSVFLFPPKP | NO: | YQQLPGTAP | FYPGAVTV |
| | 588) | EWVSAIRPS | KDTLMISRTPEVTCVVVDVSDEDGEV | 588) | KLLIYKNHQ | AWKADSSP |
| | | GSVTYYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SASKSGTSA | TPSKQSNN |
| | | DNSKNTLYL | KALPRPIEKTISKAKGQPREPQVYTL | | SLAISGLQS | KYAASSYL |
| | | QMNSLRGED | PPSRDELTKNQVSLTCLVKGFYPSDI | | EDEADYYCA | SLTPEQWK |
| | | TAIYYCATE | AVEWESNGQPENNYKTTPPVLDSDGS | | AWDDSLNVR | SHRSYSCQ |
| | | ASYSVFDWG | FFLYSKLTVDKSRWQQGNVFSCSVMH | | VFGGGTKLT | VTHEGSTV |
| | | LGTLVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 231) | | | 352) | ID NO: |
| | | | | | | 416) |
| TA186 | MGWSCII | DVQLVESGG | ASTKGPSVFPLAPSSKSTSGGTAALG | MGWSCII | QSVLTQPPS | GQPKAAPS |
| | LFLVATA | GLVQPGGSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSS | EELQANKA |
| | (SEQ ID | TFNSYDMNW | QTYICNVNHKPSNTKVDKKVEPKSCD | (SEQ ID | NIGTRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVSTISGD | KDTLMISRTPEVTCVVVDVSDEDGEV | 588) | KLLIYRNFE | AWKADSSP |
| | | GGDIYYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCA | SLTPEQWK |
| | | TAIYYCGRE | AVEWESNGQPENNYKTTPPVLDSDGS | | SWDDSLNGW | SHRSYSCQ |
| | | GLNAFSDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 232) | | | 353) | ID NO: |
| | | | | | | 416) |
| TA187 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSS | EELQANKA |
| | (SEQ ID | TFSNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DDSTYYADS | KFNWYVDGVEVHNAKTKPREEQYNST | | RPSGVPDRF | VKAGVETT |
| | | VKGRATIFR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLLSLRAED | PPSRDELTKNQVSLTCLVKGFYPSDI | | EDEAYYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 233) | | | 354) | ID NO: |
| | | | | | | 416) |
| TA188 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCIASGF | FPAVLQSSGLYSLSSVVTVPSSSLGT | TGVHS | TISCSGTIF | EELQANKA |
| | (SEQ ID | TFSTNDMSW | QTYICNVNHKPSNTKVDKKVEPKSCD | (SEQ ID | NIGTNYVSW | TLVCLISD |
| | NO: | VRQAPGEGL | KTHTCPPCPAPDLLGDDSVFLFPPKP | NO: | YQQLPGTAP | FYPGAVTV |
| | 588) | EWVSAIRPS | KDTLMISRTPEVTCVVVDVSDEDGEV | 588) | KLLIYLNSQ | AWKADSSP |
| | | GSVTYYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SASKSGTSA | TPSKQSNN |
| | | DNSKNTLYL | KALPRPIEKTISKAKGQPREPQVYTL | | SLAISGLQS | KYAASSYL |
| | | QMNSLRGED | PPSRDELTKNQVSLTCLVKGFYPSDI | | EDEADYYCA | SLTPEQWK |
| | | TAIYYCARE | AVEWESNGQPENNYKTTPPVLDSDGS | | AWDDSLNVR | SHRSYSCQ |
| | | ASYSVEDWG | FFLYSKLTVDKSRWQQGNVFSCSVMH | | VFGGGTKLT | VTHEGSTV |
| | | LGTLVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 234) | | | 355) | ID NO: |
| | | | | | | 416) |
| TA189 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFSNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DYTTYYAAS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFSLRAED | PPSRDELTKNQVSLTCLVKGFYPSDI | | EDEADYYCA | SLTPEQWK |
| | | TAIYYCTRE | AVEWESNGQPENNYKTTPPVLDSDGS | | AWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 235) | | | 356) | ID NO: |
| | | | | | | 416) |
| TA190 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | VSGTPGQRV | VTLFPPSS |
| | TGVHS | RLSCTASGF | FPAVLQSSGLYSLSSVVTVPSSSLGT | TGVHS | TISCSGTIS | EELQANKA |
| | (SEQ ID | SFYNYAMNW | QTYICNVNHKPSNTKVDKKVEPKSCD | (SEQ ID | NIGNDNRVH | TLVCLISD |
| | NO: | VRQAPGEGL | KTHTCPPCPAPDLLGDDSVFLFPPKP | NO: | WYQQLPGTA | FYPGAVTV |
| | 588) | EFVADISGS | KDTLMISRTPEVTCVVVDVSDEDGEV | 588) | PKLLIYGNH | AWKADSSP |
| | | GDTTSYAPA | KFNWYVDGVEVHNAKTKPREEQYNST | | QRPSGVPDR | VKAGVETT |
| | | VKGRFTISR | YRVVSVLTVLHQDWLNGKEYKCKVSN | | FSGSKSGTS | TPSKQSNN |
| | | DNSKNTLYL | KALPRPIEKTISKAKGQPREPQVYTL | | ASLAISGLQ | KYAASSYL |
| | | QMNSLRAED | PPSRDELTKNQVSLTCLVKGFYPSDI | | SEDEADYYC | SLTPEQWK |
| | | TAIYYCAKD | AVEWESNGQPENNYKTTPPVLDSDGS | | GTWDDSLNT | SHRSYSCQ |
| | | SGDILFDYW | FFLYSKLTVDKSRWQQGNVFSCSVMH | | WVFGGGTKL | VTHEGSTV |
| | | GLGTLVTVS | EALHNHYTQKSLSLSPG (SEQ ID | | TVL (SEQ | EKTVAPTE |
| | | S (SEQ ID | NO: 587) | | ID NO: | CS (SEQ |
| | | NO: 236) | | | 357) | ID NO: |
| | | | | | | 416) |
| TA191 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGTTF | EELQANKA |
| | (SEQ ID | TFSNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGI | KDTLMISRTPEVTCVVVDVSDEDGEV | 588) | KLLIYRNFE | AWKADSSP |
| | | DDSTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 237) | | | 358) | ID NO: |
| | | | | | | 416) |
| TA192 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSYS | EELQANKA |
| | (SEQ ID | TFSSYAMNW | QTYICNVNHKPSNTKVDKKVEPKSCD | (SEQ ID | NIGNNYVYW | TLVCLISD |
| | NO: | VRQAPGEGL | KTHTCPPCPAPDLLGDDSVFLFPPKP | NO: | YQQLPGTAP | FYPGAVTV |
| | 588) | EYVADISGS | KDTLMISRTPEVTCVVVDVSDEDGEV | 588) | KLLIYKDDQ | AWKADSSP |
| | | GDATGYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCG | SLTPEQWK |
| | | TAIYYCAKD | AVEWESNGQPENNYKTTPPVLDSDGS | | AFDDSLNVW | SHRSYSCQ |
| | | SGDIFFDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 238) | | | 359) | ID NO: |
| | | | | | | 416) |
| TA193 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFRNYDMAW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DGTTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLLSLRDED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 239) | | | 328) | ID NO: |
| | | | | | | 416) |
| TA194 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFRNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DDTTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GPGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 240) | | | 328) | ID NO: |
| | | | | | | 416) |
| TA195 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSS | EELQANKA |
| | (SEQ ID | TFSNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DDSTYYADS | KFNWYVDGVEVHNAKTKPREEQYNST | | RPSGVPDRF | VKAGVETT |
| | | VKGRATIFR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLLSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 233) | | | 327) | ID NO: |
| | | | | | | 416) |
| TA196 | MGWSCII | DVQLVESGG | ASTKGPSVFPLAPSSKSTSGGTAALG | MGWSCII | QSVLTQPPS | GQPKAAPS |
| | LFLVATA | GVVRPGDSL | CLVKDYFPEPVTVSWNSGALTSGVHT | LFLVATA | ASGTPGQRV | VTLFPPSS |
| | TGVHS | TLSCAASGF | FPAVLQSSGLYSLSSVVTVPSSSLGT | TGVHS | TISCSGSTF | EELQANKA |
| | (SEQ ID | TFSNYAMSW | QTYICNVNHKPSNTKVDKKVEPKSCD | (SEQ ID | NIQSNYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVASISTN | KDTLMISRTPEVTCVVVDVSDEDGEV | 588) | KLLIYQSHV | AWKADSSP |
| | | GGSTGYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | RLFSLRAED | PPSRDELTKNQVSLTCLVKGFYPSDI | | EDEADYYCS | SLTPEQWK |
| | | TAIYYCTKE | AVEWESNGQPENNYKTTPPVLDSDGS | | SWDASLNGW | SHRSYSCQ |
| | | TWNTSFDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 241) | | | 360) | ID NO: |
| | | | | | | 416) |
| TA197 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSS | EELQANKA |
| | (SEQ ID | TFSNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVTTISGT | KDTLMISRTPEVTCVVVDVSDEDGEV | 588) | KLLIYRNFE | AWKADSSP |
| | | DDSTYYADS | KFNWYVDGVEVHNAKTKPREEQYNST | | RPSGVPDRF | VKAGVETT |
| | | VKGRATIFR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLLSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 242) | | | 327) | ID NO: |
| | | | | | | 416) |
| TA198 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFSNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DYTTYYAAS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | AWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 243) | | | 356) | ID NO: |
| | | | | | | 416) |
| TA199 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGRRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | NFSSYDMNW | QTYICNVNHKPSNTKVDKKVEPKSCD | (SEQ ID | NIGTRYVYW | TLVCLISD |
| | NO: | VRQAPGEGL | KTHTCPPCPAPDLLGDDSVFLFPPKP | NO: | YQQLPGTAP | FYPGAVTV |
| | 588) | QWVATITAA | KDTLMISRTPEVTCVVVDVSDEDGEV | 588) | KLLIYRNFE | AWKADSSP |
| | | GDITYYADS | KFNWYVDGVEVHNAKTKPREEQYNST | | RPSGVPDRF | VKAGVETT |
| | | VRGRFAISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | GLSSLRAED | PPSRDELTKNQVSLTCLVKGFYPSDI | | EDEADYYCA | SLTPEQWK |
| | | TAIYYCGRE | AVEWESNGQPENNYKTTPPVLDSDGS | | AWDDRVNGW | SHRSYSCQ |
| | | PWNSFSDYW | FFLYSKLTVDKSRWQQGNVFSCSVMH | | LFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 158) | | | 361) | ID NO: |
| | | | | | | 416) |
| TA200 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGGSS | EELQANKA |
| | (SEQ ID | TFSNFYMNW | QTYICNVNHKPSNTKVDKKVEPKSCD | (SEQ ID | NIGTNTVDW | TLVCLISD |
| | NO: | VRQAPGEGL | KTHTCPPCPAPDLLGDDSVFLFPPKP | NO: | YQQLPGTAP | FYPGAVTV |
| | 588) | EWVSTISGI | KDTLMISRTPEVTCVVVDVSDEDGEV | 588) | KLLIYDNFE | AWKADSSP |
| | | DDTTWYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSRNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCG | SLTPEQWK |
| | | TATYYCAKG | AVEWESNGQPENNYKTTPPVLDSDGS | | TWDDSLNAW | SHRSYSCQ |
| | | TDFLDYWGL | FFLYSKLTVDKSRWQQGNVFSCSVMH | | VFGGGTKLT | VTHEGSTV |
| | | GTLVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 244) | | | 362) | ID NO: |
| | | | | | | 416) |
| TA201 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSNS | EELQANKA |
| | (SEQ ID | TFSDYDMAW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DDSTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 230) | | | 363) | ID NO: |
| | | | | | | 416) |
| TA202 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TMSCSGSSS | EELQANKA |
| | (SEQ ID | DFSNYDMNW | QTYICNVNHKPSNTKVDKKVEPKSCD | (SEQ ID | NIGNRYVYW | TLVCLISD |
| | NO: | VRQAPGEGL | KTHTCPPCPAPDLLGDDSVFLFPPKP | NO: | YQQFPGTAP | FYPGAVTV |
| | 588) | EWVATISGS | KDTLMISRTPEVTCVVVDVSDEDGEV | 588) | KLLIHHNSQ | AWKADSSP |
| | | ASITGTANI | KFNWYVDGVEVHNAKTKPREEQYNST | | RPSGVPDRF | VKAGVETT |
| | | TYYAPAVKG | YRVVSVLTVLHQDWLNGKEYKCKVSN | | SGSKSGTSA | TPSKQSNN |
| | | RFTISRDNS | KALPRPIEKTISKAKGQPREPQVYTL | | SLAISGLQS | KYAASSYL |
| | | KNTLYLRLF | PPSRDELTKNQVSLTCLVKGFYPSDI | | EDEADYYCA | SLTPEQWK |
| | | SLRAEDTAI | AVEWESNGQPENNYKTTPPVLDSDGS | | SWDDSLNGW | SHRSYSCQ |
| | | YYCARETED | FFLYSKLTVDKSRWQQGNVFSCSVMH | | VFGGGTKLT | VTHEGSTV |
| | | GFFDYCGLG | EALHNHYTQKSLSLSPG (SEQ ID | | VL (SEQ | EKTVAPTE |
| | | TLVTVSS | NO: 587) | | ID NO: | CS (SEQ |
| | | (SEQ ID | | | 364) | ID NO: |
| | | NO: 245) | | | | 416) |
| TA203 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFRNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNSQ | AWKADSSP |
| | | DDTTYYADS | KFNWYVDGVEVHNAKTKPREEQYNST | | RPSGVPDRF | VKAGVETT |
| | | VKGRAAISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLLSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 246) | | | 365) | ID NO: |
| | | | | | | 416) |
| TA204 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSS | EELQANKA |
| | (SEQ ID | TFSNFYMNW | QTYICNVNHKPSNTKVDKKVEPKSCD | (SEQ ID | NIGTNTVDW | TLVCLISD |
| | NO: | VRQAPGEGL | KTHTCPPCPAPDLLGDDSVFLFPPKP | NO: | YQQLPGTAP | FYPGAVTV |
| | 588) | EWVSTISGI | KDTLMISRTPEVTCVVVDVSDEDGEV | 588) | KLLIYDNFE | AWKADSSP |
| | | DDTTWYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSRNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCG | SLTPEQWK |
| | | TATYYCAKG | AVEWESNGQPENNYKTTPPVLDSDGS | | TWDDSLNAW | SHRSYSCQ |
| | | TDFLDYWGL | FFLYSKLTVDKSRWQQGNVFSCSVMH | | VFGGGTKLT | VTHEGSTV |
| | | GTLVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 244) | | | 366) | ID NO: |
| | | | | | | 416) |
| TA205 | MGWSCII | DVQLVESGG | ASTKGPSVFPLAPSSKSTSGGTAALG | MGWSCII | QSVLTQPPS | GQPKAAPS |
| | LFLVATA | GMVRPGESL | CLVKDYFPEPVTVSWNSGALTSGVHT | LFLVATA | ASGTPGQRV | VTLFPPSS |
| | TGVHS | RVSCAASGF | FPAVLQSSGLYSLSSVVTVPSSSLGT | TGVHS | TISCSGSNS | EELQANKA |
| | (SEQ ID | DFSNYHMNW | QTYICNVNHKPSNTKVDKKVEPKSCD | (SEQ ID | NIGDHYVDW | TLVCLISD |
| | NO: | VRQAPGEGL | KTHTCPPCPAPDLLGDDSVFLFPPKP | NO: | YQQLPGTAP | FYPGAVTV |
| | 588) | EWVSRISDG | KDTLMISRTPEVTCVVVDVSDEDGEV | 588) | KLLIYNNVQ | AWKADSSP |
| | | DSRTYYSDF | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFSLRADD | PPSRDELTKNQVSLTCLVKGFYPSDI | | EDEADYYCA | SLTPEQWK |
| | | TAIYYCVRE | AVEWESNGQPENNYKTTPPVLDSDGS | | TWDDNLNGW | SHRSYSCQ |
| | | TLNNVFDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 247) | | | 367) | ID NO: |
| | | | | | | 416) |
| TA206 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSY | EELQANKA |
| | (SEQ ID | TFSSHGMNW | QTYICNVNHKPSNTKVDKKVEPKSCD | (SEQ ID | NIDYNYIDW | TLVCLISD |
| | NO: | LRQAPGEGL | KTHTCPPCPAPDLLGDDSVFLFPPKP | NO: | YQQLPGTAP | FYPGAVTV |
| | 588) | ESVAGIRSD | KDTLMISRTPEVTCVVVDVSDEDGEV | 588) | KLLIYNSDQ | AWKADSSP |
| | | GKYIYYADS | KFNWYVDGVEVHNAKTKPREEQYNST | | RPSGVPDRF | VKAGVETT |
| | | VKGRATISR | YRVVSVLIVLHQDWLNGKEYKCKVSN | | SGSKSGTSA | TPSKQSNN |
| | | DDSKSTVYL | KALPRPIEKTISKAKGQPREPQVYTL | | SLAISGLQS | KYAASSYL |
| | | QMDSLRAED | PPSRDELTKNQVSLTCLVKGFYPSDI | | EDEADYYCA | SLTPEQWK |
| | | TAIYYCARG | AVEWESNGQPENNYKTTPPVLDSDGS | | SWDAGLNVW | SHRSYSCQ |
| | | WNFFDYWGP | FFLYSKLTVDKSRWQQGNVFSCSVMH | | MFGGGTKLT | VTHEGSTV |
| | | GALVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 248) | | | 368) | ID NO: |
| | | | | | | 416) |
| TA207 | MGWSCII | DVQLVDSGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFRNYDMAW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DGTTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLLSLRDED | PPSRDELTKNQVSLTCLVKGFYPSDI | | EDEADYYCA | SLTPEQWK |
| | | TAIYYCARE | AVEWESNGQPENNYKTTPPVLDSDGS | | SWDERLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 249) | | | 369) | ID NO: |
| | | | | | | 416) |
| TA208 | MGWSCII | DVQSVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | NFSSYDMNW | QTYICNVNHKPSNTKVDKKVEPKSCD | (SEQ ID | NIGTRYVYW | TLVCLISD |
| | NO: | VRQAPGEGL | KTHTCPPCPAPDLLGDDSVFLFPPKP | NO: | YQQLPGTAP | FYPGAVTV |
| | 588) | QWVATITAA | KDTLMISRTPEVTCVVVDVSDEDGEV | 588) | KLLIYRNFE | AWKADSSP |
| | | GDITYYADS | KFNWYVDGVEVHNAKTKPREEQYNST | | RPSGVPDRF | VKAGVETT |
| | | VRGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | GLSSLRAED | PPSRDELTKNQVSLTCLVKGFYPSDI | | EDEADYYCA | SLTPEQWK |
| | | TAIYYCGRE | AVEWESNGQPENNYKTTPPVLDSDGS | | AWDDRVNGW | SHRSYSCQ |
| | | PWNSFSDYW | FFLYSKLTVDKSRWQQGNVFSCSVMH | | LFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 250) | | | 348) | ID NO: |
| | | | | | | 416) |
| TA209 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | NCSSYDMNW | QTYICNVNHKPSNTKVDKKVEPKSCD | (SEQ ID | NIGTRYVYW | TLVCLISD |
| | NO: | VRQAPGEGL | KTHTCPPCPAPDLLGDDSVFLFPPKP | NO: | YQQLPGTAP | FYPGAVTV |
| | 588) | QWVATITAA | KDTLMISRTPEVTCVVVDVSDEDGEV | 588) | KLLIYRNFE | AWKADSSP |
| | | GDITYYADS | KFNWYVDGVEVHNAKTKPREEQYNST | | RPSGVPDRF | VKAGVETT |
| | | VRGRFAISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | GLSSLRAED | PPSRDELTKNQVSLTCLVKGFYPSDI | | EDEADYYCA | SLTPEQWK |
| | | TAIYYCGRE | AVEWESNGQPENNYKTTPPVLDSDGS | | AWDDRVNGW | SHRSYSCQ |
| | | PWNSFSDYW | FFLYSKLTVDKSRWQQGNVFSCSVMH | | LFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 251) | | | 348) | ID NO: |
| | | | | | | 416) |
| TA210 | MGWSCII | DVQLVESGG | ASTKGPSVFPLAPSSKSTSGGTAALG | MGWSCII | QSVLTQPPS | GQPKAAPS |
| | LFLVATA | GVVRLGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | NFIDYDMAW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DDSTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQA | KYAASSYL |
| | | RLFSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIGYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 252) | | | 370) | ID NO: |
| | | | | | | 416) |
| TA211 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSNS | EELQANKA |
| | (SEQ ID | TFSDYDMAW | QTYICNVNHKPSNTKVDKKVEPKSCD | (SEQ ID | SIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DDSTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 230) | | | 371) | ID NO: |
| | | | | | | 416) |
| TA212 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFRNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQVPGTAP | FYPGAVTV |
| | 588) | EWVATISAT | KDTLMISRTPEVTCVVVDVSDEDGEV | 588) | KLLIYRNFE | AWKADSSP |
| | | DDTTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFNLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFSGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 253) | | | 372) | ID NO: |
| | | | | | | 416) |
| TA213 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | NFSSYDMNW | QTYICNVNHKPSNTKVDKKVEPKSCD | (SEQ ID | NIGTRYVYW | TLVCLISD |
| | NO: | VRQAPGEGP | KTHTCPPCPAPDLLGDDSVFLFPPKP | NO: | YQQLPGTAP | FYPGAVTV |
| | 588) | QWVATITAA | KDTLMISRTPEVTCVVVDVSDEDGEV | 588) | KLLIYRNFE | AWKADSSP |
| | | GDITYYADS | KFNWYVDGVEVHNAKTKPREEQYNST | | RPSGVPDRF | VKAGVETT |
| | | VRGRFAISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | GLSSLRAED | PPSRDELTKNQVSLTCLVKGFYPSDI | | EDEADYYCA | SLTPEQWK |
| | | TAIYYCGRE | AVEWESNGQPENNYKTTPPVLDSDGS | | AWDDRVNGW | SHRSYSCQ |
| | | PWNSFSDYW | FFLYSKLTVDKSRWQQGNVFSCSVMH | | LFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 254) | | | 348) | ID NO: |
| | | | | | | 416) |
| TA214 | MGWSCII | DVQLVESGG | ASTKGPSVFPLAPSSKSTSGGTAALG | MGWSCII | QSVLTQQPA | GQPKAAPS |
| | LFLVATA | GVVRPGESL | CLVKDYFPEPVTVSWNSGALTSGVHT | LFLVATA | PVSGTPGQR | VTLFPPSS |
| | TGVHS | RLSCAASGF | FPAVLQSSGLYSLSSVVTVPSSSLGT | TGVHS | VTISCSGSS | EELQANKA |
| | (SEQ ID | DFINYVMNW | QTYICNVNHKPSNTKVDKKVEPKSCD | (SEQ ID | SNIGDHYVD | TLVCLISD |
| | NO: | VRQAPGEGL | KTHTCPPCPAPDLLGDDSVFLFPPKP | NO: | WYQQLPGTA | FYPGAVTV |
| | 588) | EFVARITNS | KDTLMISRTPEVTCVVVDVSDEDGEV | 588) | PKLLIYDNS | AWKADSSP |
| | | GDRTWYADS | KFNWYVDGVEVHNAKTKPREEQYNST | | QRPSGVPDR | VKAGVETT |
| | | VKGRFTISR | YRVVSVLTVLHQDWLNGKEYKCKVSN | | FSGSKSGTS | TPSKQSNN |
| | | DNSNNTLYL | KALPRPIEKTISKAKGQPREPQVYTL | | ASLAISGLQ | KYAASSYL |
| | | RLFSLRAED | PPSRDELTKNQVSLTCLVKGFYPSDI | | SEDEADYYC | SLTPEQWK |
| | | TAIYYCVRE | AVEWESNGQPENNYKTTPPVLDSDGS | | GTWDDDLNV | SHRSYSCQ |
| | | TSNYLLDYW | FFLYSKLTVDKSRWQQGNVFSCSVMH | | WVFGGGTKL | VTHEGSTV |
| | | GLGTLVTVS | EALHNHYTQKSLSLSPG (SEQ ID | | TVL (SEQ | EKTVAPTE |
| | | S (SEQ ID | NO: 587) | | ID NO: | CS (SEQ |
| | | NO: 255) | | | 373) | ID NO: |
| | | | | | | 416) |
| TA215 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | GLSCAASGF | FPAVLQSSGLYSLSSVVTVPSSSLGT | TGVHS | TISCSGSTS | EELQANKA |
| | (SEQ ID | TFRNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQFPGTAP | FYPGAVTV |
| | 588) | GWVATISGT | KDTLMISRTPEVTCVVVDVSDEDGEV | 588) | KLLIYRNSQ | AWKADSSP |
| | NO: | DDTTYYADS | KFNWYVDGVEVHNAKTKPREEQYNST | | RPSGVPDRF | VKAGVETT |
| | | VKGRAAISR | YRVVSVLTVLHQDWLNGKEYKCKVSN | | SGSKSGTSA | TPSKQSNN |
| | 588) | DNSNNTLYL | KALPRPIEKTISKAKGQPREPQVYTL | | SLAISGLQS | KYAASSYL |
| | | RLLSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 256) | | | 374) | ID NO: |
| | | | | | | 416) |
| TA216 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TMSCSGSSS | EELQANKA |
| | (SEQ ID | DFSNYDMNW | QTYICNVNHKPSNTKVDKKVEPKSCD | (SEQ ID | NIGNRYVYW | TLVCLISD |
| | NO: | VRQAPGEGL | KTHTCPPCPAPDLLGDDSVFLFPPKP | NO: | YRQFPGTAP | FYPGAVTV |
| | 588) | EWVATISGS | KDTLMISRTPEVTCVVVDVSDEDGEV | 588) | KLLIHHNSQ | AWKADSSP |
| | | ASITGTANI | KFNWYVDGVEVHNAKTKPREEQYNST | | RPSGVPDRF | VKAGVETT |
| | | TYYAPAVKG | YRVVSVLTVLHQDWLNGKEYKCKVSN | | SGSKSGTSA | TPSKQSNN |
| | | RFTISRDNS | KALPRPIEKTISKAKGQPREPQVYTL | | SLAISGLQS | KYAASSYL |
| | | KNTLYLRLF | PPSRDELTKNQVSLTCLVKGFYPSDI | | EDEADYYCA | SLTPEQWK |
| | | SLRAEDTAI | AVEWESNGQPENNYKTTPPVLDSDGS | | SWDDSLNGW | SHRSYSCQ |
| | | YYCARETFD | FFLYSKLTVDKSRWQQGNVFSCSVMH | | VFGGGTKLT | VTHEGSTV |
| | | GFFDYCGLG | EALHNHYTQKSLSLSPG (SEQ ID | | VL (SEQ | EKTVAPTE |
| | | TLVTVSS | NO: 587) | | ID NO: | CS (SEQ |
| | | (SEQ ID | | | 375) | ID NO: |
| | | NO: 245) | | | | 416) |
| TA217 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | NFSSYDMNW | QTYICNVNHKPSNTKVDKKVEPKSCD | (SEQ ID | NIGTRYVYW | TLVCLISD |
| | NO: | VRQAPGEGL | KTHTCPPCPAPDLLGDDSVFLFPPKP | NO: | YQQLPGTAP | FYPGAVTV |
| | 588) | QWVATITAA | KDTLMISRTPEVTCVVVDVSDEDGEV | 588) | KLLIYRNFE | AWKADSSP |
| | | GDITYYADS | KFNWYVDGVEVHNAKTKPREEQYNST | | RPSGVPDRF | VKAGVETT |
| | | VRGRFAISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | GLSSLRAKD | PPSRDELTKNQVSLTCLVKGFYPSDI | | EDEADYYCA | SLTPEQWK |
| | | TAIYYCGRE | AVEWESNGQPENNYKTTPPVLDSDGS | | AWDDRVNGW | SHRSYSCQ |
| | | PWNSFSDYW | FFLYSKLTVDKSRWQQGNVFSCSVMH | | LFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 257) | | | 348) | ID NO: |
| | | | | | | 416) |
| TA218 | MGWSCII | DVQLVESGG | ASTKGPSVFPLAPSSKSTSGGTAALG | MGWSCII | QSVLTQPPS | GQPKAAPS |
| | LFLVATA | GVVRPGDSL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSSS | EELQANKA |
| | (SEQ ID | TFSDYDMTW | QTYICNVNHKPSNTKVDKKVEPKSCD | (SEQ ID | NIGTRYVYW | TLVCLISD |
| | NO: | VRQAPGEGL | KTHTCPPCPAPDLLGDDSVFLFPPKP | NO: | YQQVPGTAP | FYPGAVTV |
| | 588) | EWVSTISGS | KDTLMISRTPEVTCVVVDVSDEDGEV | 588) | KLLIYRNFE | AWKADSSP |
| | | GDRIYYADS | KFNWYVDGVEVHNAKTKPREEQYNST | | RPSGVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | RLFSLRAED | PPSRDELTKNQVSLTCLVKGFYPSDI | | EDEADYYCA | SLTPEQWK |
| | | TAIYYCAKE | AVEWESNGQPENNYKTTPPVLDSDGS | | AWDDSLNGW | SHRSYSCQ |
| | | GWNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 258) | | | 376) | ID NO: |
| | | | | | | 416) |
| TA219 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGGTS | EELQANKA |
| | (SEQ ID | NFSSYDMNW | QTYICNVNHKPSNTKVDKKVEPKSCD | (SEQ ID | NIGTRYVYW | TLVCLISD |
| | NO: | VRQAPGEGL | KTHTCPPCPAPDLLGDDSVFLFPPKP | NO: | YQQLPGTAP | FYPGAVTV |
| | 588) | QWVATITAA | KDTLMISRTPEVTCVVVDVSDEDGEV | 588) | KLLIYRNFE | AWKADSSP |
| | | GDITYYADS | KFNWYVDGVEVHNAKTKPREEQYNST | | RPSGVPDRF | VKAGVETT |
| | | VRGRFAISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | GLSSLRAED | PPSRDELTKNQVSLTCLVKGFYPSDI | | EDEADYYCA | SLTPEQWK |
| | | TAIYYCGRE | AVEWESNGQPENNYKTTPPVLDSDGS | | AWDDRVNGW | SHRSYSCQ |
| | | PWNSFSDYW | FFLYSKLTVDKSRWQQGNVFSCSVMH | | LFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 158) | | | 377) | ID NO: |
| | | | | | | 416) |
| TA220 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGS | FPAVLQSSGLYSLSSVVTVPSSSLGT | TGVHS | TISCSGSTS | EELQANKA |
| | (SEQ ID | NFSSYDMNW | QTYICNVNHKPSNTKVDKKVEPKSCD | (SEQ ID | NIGTRYVYW | TLVCLISD |
| | NO: | VRQAPGEGL | KTHTCPPCPAPDLLGDDSVFLFPPKP | NO: | YQQLPGTAP | FYPGAVTV |
| | 588) | QWVATITAA | KDTLMISRTPEVTCVVVDVSDEDGEV | 588) | KLLIYRNFE | AWKADSSP |
| | | GDITYYADS | KFNWYVDGVEVHNAKTKPREEQYNST | | RPSGVPDRF | VKAGVETT |
| | | VRGRFAISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | GLSSLRAED | PPSRDELTKNQVSLTCLVKGFYPSDI | | EDEADYYCA | SLTPEQWK |
| | | TAIYYCGRE | AVEWESNGQPENNYKTTPPVLDSDGS | | AWDDRVNGW | SHRSYSCQ |
| | | PWNSFSDYW | FFLYSKLTVDKSRWQQGNVFSCSVMH | | LFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 259) | | | 348) | ID NO: |
| | | | | | | 416) |
| TA221 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFSNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DYTTYYAAS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFSLRAED | PPSRDELTKNQVSLTCLVKGFYPSDI | | EDEADYYCA | SLTPEQWK |
| | | TAIYYCTRE | AVEWESNGQPENNYKTTPPVLDSDGS | | AWDESLNGW | SHRSYSCQ |
| | | APNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 260) | | | 356) | ID NO: |
| | | | | | | 416) |
| TA222 | MGWSCII | DVQLVESGG | ASTKGPSVFPLAPSSKSTSGGTAALG | MGWSCII | QSVLTQQPA | GQPKAAPS |
| | LFLVATA | GVVRPGESL | CLVKDYFPEPVTVSWNSGALTSGVHT | LFLVATA | SVSGTPGQR | VTLFPPSS |
| | TGVHS | RLSCAASGF | FPAVLQSSGLYSLSSVVTVPSSSLGT | TGVHS | VTISCSGSS | EELQANKA |
| | (SEQ ID | DFINYVMNW | QTYICNVNHKPSNTKVDKKVEPKSCD | (SEQ ID | SNIGDHYVD | TLVCLISD |
| | NO: | VRQAPGEGL | KTHTCPPCPAPDLLGDDSVFLFPPKP | NO: | WYQQLPGTA | FYPGAVTV |
| | 588) | EFVARITNS | KDTLMISRTPEVTCVVVDVSDEDGEV | 588) | PKLLIYDNS | AWKADSSP |
| | | GDRTWYADS | KFNWYVDGVEVHNAKTKPREEQYNST | | QRPSGVPDR | VKAGVETT |
| | | VKGRFAISR | YRVVSVLTVLHQDWLNGKEYKCKVSN | | FSGSKSGTS | TPSKQSNN |
| | | DNSNNTLYL | KALPRPIEKTISKAKGQPREPQVYTL | | ASLAISGLQ | KYAASSYL |
| | | RLFSLRAED | PPSRDELTKNQVSLTCLVKGFYPSDI | | SEDEADYYC | SLTPEQWK |
| | | TAIYYCVRE | AVEWESNGQPENNYKTTPPVLDSDGS | | GTWDDDLNV | SHRSYSCQ |
| | | TSNYLLDYW | FFLYSKLTVDKSRWQQGNVFSCSVMH | | WVFGGGTKL | VTHEGSTV |
| | | GLGTLVTVS | EALHNHYTQKSLSLSPG (SEQ ID | | TVL (SEQ | EKTVAPTE |
| | | S (SEQ ID | NO: 587) | | ID NO: | CS (SEQ |
| | | NO: 261) | | | 378) | ID NO: |
| | | | | | | 416) |
| TA223 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | NFIDYDMAW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLFIYRNFE | AWKADSSP |
| | | DDSTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQA | KYAASSYL |
| | | RLFSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 262) | | | 379) | ID NO: |
| | | | | | | 416) |
| TA224 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | NFSSYDMNW | QTYICNVNHKPSNTKVDKKVEPKSCD | (SEQ ID | NIGTRYVYW | TLVCLISD |
| | NO: | VRQAPGEGL | KTHTCPPCPAPDLLGDDSVFLFPPKP | NO: | YQQLPGTAP | FYPGAVTV |
| | 588) | QWVATITAA | KDTLMISRTPEVTCVVVDVSDEDGEV | 588) | KLLIYRNFE | AWKADSSP |
| | | GDITYYAGS | KFNWYVDGVEVHNAKTKPREEQYNST | | RPSGVPDRF | VKAGVETT |
| | | VRGRFAISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | GLSSLRAED | PPSRDELTKNQVSLTCLVKGFYPSDI | | EDEADYYCA | SLTPEQWK |
| | | TAIYYCGRE | AVEWESNGQPENNYKTTPPVLDSDGS | | AWDDRVNGW | SHRSYSCQ |
| | | PWNSFSDYW | FFLYSKLTVDKSRWQQGNVFSCSVMH | | LFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 263) | | | 348) | ID NO: |
| | | | | | | 416) |
| TA225 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCVASGF | FPAVLQSSGLYSLSSVVTVPSSSLGT | TGVHS | TISCSGSNS | EELQANKA |
| | (SEQ ID | TFGSDGMNW | QTYICNVNHKPSNTKVDKKVEPKSCD | (SEQ ID | NIDYNYIDW | TLVCLISD |
| | NO: | VRQAPGKGL | KTHTCPPCPAPDLLGDDSVFLFPPKP | NO: | YQQLPGTAP | FYPGAVTV |
| | 588) | DWISTISID | KDTLMISRTPEVTCVVVDVSDEDGEV | 588) | KLLIYNNDQ | AWKADSSP |
| | | GTPTYYAES | KFNWYVDGVEVHNAKTKPREEQYNST | | RPSEVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCG | SLTPEQWK |
| | | TAIYYCVKG | AVEWESNGQPENNYKTTPPVLDSDGS | | TWDDSLNAW | SHRSYSCQ |
| | | YNFFDYWGL | FFLYSKLTVDKSRWQQGNVFSCSVMH | | VFGGGTKLT | VTHEGSTV |
| | | GTLVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 264) | | | 380) | ID NO: |
| | | | | | | 416) |
| TA226 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | NFIDYDMAW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DDSTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQA | KYAASSYL |
| | | RLFSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 262) | | | 370) | ID NO: |
| | | | | | | 416) |
| TA227 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | NFIDYDMAW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DDSTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | AWDDSLNAW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 262) | | | 381) | ID NO: |
| | | | | | | 416) |
| TA228 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSNS | EELQANKA |
| | (SEQ ID | TFSDYDMAW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQLPGTAP | FYPGAVTV |
| | 588) | EWVATISGT | KDTLMISRTPEVTCVVVDVSDEDGEV | 588) | KLLIYRNFE | AWKADSSP |
| | | DDSTCYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 265) | | | 363) | ID NO: |
| | | | | | | 416) |
| TA229 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | NFSSYDMNW | QTYICNVNHKPSNTKVDKKVEPKSCD | (SEQ ID | NIRTRYVYW | TLVCLISD |
| | NO: | VRQAPGEGL | KTHTCPPCPAPDLLGDDSVFLFPPKP | NO: | YQQLPGTAP | FYPGAVTV |
| | 588) | QWVATITAA | KDTLMISRTPEVTCVVVDVSDEDGEV | 588) | KLLIYRNFE | AWKADSSP |
| | | GDITYYADS | KFNWYVDGVEVHNAKTKPREEQYNST | | RPSGVPDRF | VKAGVETT |
| | | VRGRFAISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | GLSSLRAED | PPSRDELTKNQVSLTCLVKGFYPSDI | | EDEADYYCA | SLTPEQWK |
| | | TAIYYCGRE | AVEWESNGQPENNYKTTPPVLDSDGS | | AWDDRVNGW | SHRSYSCQ |
| | | PWNSFSDYW | FFLYSKLTVDKSRWQQGNVFSCSVMH | | LFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 158) | | | 382) | ID NO: |
| | | | | | | 416) |
| TA230 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | NFSSYDMNW | QTYICNVNHKPSNTKVDKKVEPKSCD | (SEQ ID | NIGTRYVYW | TLVCLISD |
| | NO: | VRQAPGEGL | KTHTCPPCPAPDLLGDDSVFLFPPKP | NO: | YQQLPGTAP | FYPGAVTV |
| | 588) | QWVATITAA | KDTLMISRTPEVTCVVVDVSDEDGEV | 588) | KLLIYRNFE | AWKADSSP |
| | | GDITYYADS | KFNWYVDGVEVHNAKTKPREEQYNST | | RPSGVPDRF | VKAGVETT |
| | | VRGRFAISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | GLSSLRAED | PPSRDELTKNQVSLTCLVKGFYPSDI | | EDEANYYCA | SLTPEQWK |
| | | TAIYYCGRE | AVEWESNGQPENNYKTTPPVLDSDGS | | AWDDSLNGW | SHRSYSCQ |
| | | PWNSFSDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 158) | | | 383) | ID NO: |
| | | | | | | 416) |
| TA231 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCVASGF | FPAVLQSSGLYSLSSVVTVPSSSLGT | TGVHS | TISCSGSNS | EELQANKA |
| | (SEQ ID | TFGSDGMNW | QTYICNVNHKPSNTKVDKKVEPKSCD | (SEQ ID | NIDYNYIDW | TLVCLISD |
| | NO: | VRQAPGKGL | KTHTCPPCPAPDLLGDDSVFLFPPKP | NO: | YQQLPGTAP | FYPGAVTV |
| | 588) | DWISTISID | KDTLMISRTPEVTCVVVDVSDEDGEV | 588) | KLLIYNNDQ | AWKADSSP |
| | | GTPTYYAES | KFNWYVDGVEVHNAKTKPREEQYNST | | RPSEVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLQS | KYAASSYL |
| | | QMNSLRAED | PPSRDELTKNQVSLTCLVKGFYPSDI | | EDEADYYCA | SLTPEQWK |
| | | TAIYYCVKG | AVEWESNGQPENNYKTTPPVLDSDGS | | AWDDNLNSW | SHRSYSCQ |
| | | YNFFDYWGL | FFLYSKLTVDKSRWQQGNVFSCSVMH | | VFGGGTKLT | VTHEGSTV |
| | | GTLVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 264) | | | 384) | ID NO: |
| | | | | | | 416) |
| TA232 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCVASGF | FPAVLQSSGLYSLSSVVTVPSSSLGT | TGVHS | TISCSGSNS | EELQANKA |
| | (SEQ ID | TFGSDGMNW | QTYICNVNHKPSNTKVDKKVEPKSCD | (SEQ ID | NIDYNYIDW | TLVCLISD |
| | NO: | VRQAPGKGL | KTHTCPPCPAPDLLGDDSVFLFPPKP | NO: | YQQLPGTAP | FYPGAVTV |
| | 588) | DWISTISID | KDTLMISRTPEVTCVVVDVSDEDGEV | 588) | KLLIYNNDQ | AWKADSSP |
| | | GTPTYYAES | KFNWYVDGVEVHNAKTKPREEQYNST | | RPSEVPDRF | VKAGVETT |
| | | VKGRFTISR | YRVVSVLTVLHQDWLNGKEYKCKVSN | | SGSKSGTSA | TPSKQSNN |
| | | DNSKNTLYL | KALPRPIEKTISKAKGQPREPQVYTL | | SLAISGLRS | KYAASSYL |
| | | QMNSLRAED | PPSRDELTKNQVSLTCLVKGFYPSDI | | EDEADYYCA | SLTPEQWK |
| | | TAIYYCVKG | AVEWESNGQPENNYKTTPPVLDSDGS | | TWDDNLNSW | SHRSYSCQ |
| | | YNFFDYWGL | FFLYSKLTVDKSRWQQGNVFSCSVMH | | VFGGGTKLT | VTHEGSTV |
| | | GTLVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 264) | | | 385) | ID NO: |
| | | | | | | 416) |
| TA233 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFRNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQVPGTAP | FYPGAVTV |
| | 588) | EWVATISAT | KDTLMISRTPEVTCVVVDVSDEDGEV | 588) | KLLIYRNFE | AWKADSSP |
| | | DDTTYYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLFNLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 253) | | | 386) | ID NO: |
| | | | | | | 416) |
| TA234 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCAASGF | FPAVLQSSGLYSLSSVVTVPSSSLGT | TGVHS | TISCSGSTS | EELQANKA |
| | (SEQ ID | TFRNYDMTW | QTYICNVNHKPSNTKVDKKVEPKSCD | (SEQ ID | NIGRRYVYW | TLVCLISD |
| | NO: | VRQAPGEGL | KTHTCPPCPAPDLLGDDSVFLFPPKP | NO: | YQQFPGTAP | FYPGAVTV |
| | 588) | EWVATISGT | KDTLMISRTPEVTCVVVDVSDEDGEV | 588) | KLLIYRNSQ | AWKADSSP |
| | | DDTTYYADS | KFNWYVDGVEVHNAKTKPREEQYNST | | RPSGVPDRF | VKAGVETT |
| | | VKGRAAISR | YRVVSVLTVLHQDWLNGKEYKCKVSN | | SGSKSGTSA | TPSKQSNN |
| | | DNSNNTLYL | KALPRPIEKTISKAKGQPREPQVYTL | | SLAISGLQS | KYAASSYL |
| | | RLLSLRAED | PPSRDELTKNQVSLTCLVKGFYPSDI | | EDEADYYCA | SLTPEQWK |
| | | TAIYYCARE | AVEWESNGQPENNYKTTPPVLDSDGS | | SWDESLNGW | SHRSYSCQ |
| | | ASNSFIDYW | FFLYSKLTVDKSRWQQGNVFSCSVMH | | VFGGGTKLT | VTHEGSTV |
| | | GLGTLVTVS | EALHNHYTQKSLSLSPG (SEQ ID | | VL (SEQ | EKTVAPTE |
| | | S (SEQ ID | NO: 587) | | ID NO: | CS (SEQ |
| | | NO: 246) | | | 374) | ID NO: |
| | | | | | | 416) |
| TA235 | MGWSCII | DVQLVESGG | ASTKGPSVFPLAPSSKSTSGGTAALG | MGWSCII | QSVLTQPPS | GQPKAAPS |
| | LFLVATA | GVVRPGESL | CLVKDYFPEPVTVSWNSGALTSGVHT | LFLVATA | ASGTPGQRV | VTLFPPSS |
| | TGVHS | RLSCIASGE | FPAVLQSSGLYSLSSVVTVPSSSLGT | TGVHS | TISCSGSTF | EELQANKA |
| | (SEQ ID | TFSSYDIEW | QTYICNVNHKPSNTKVDKKVEPKSCD | (SEQ ID | NIGNNYVSW | TLVCLISD |
| | NO: | VRQAPGKGL | KTHTCPPCPAPDLLGDDSVFLFPPKP | NO: | YQQLPGTAP | FYPGAVTV |
| | 588) | EWVAAIDDD | KDTLMISRTPEVTCVVVDVSDEDGEV | 588) | KVLIYKNLQ | AWKADSSP |
| | | GSRRWYADS | KFNWYVDGVEVHNAKTKPREEQYNST | | RPSGVPDRF | VKAGVETT |
| | | VKGRATISR | YRVVSVLTVLHQDWLNGKEYKCKVSN | | SGSKSGTSA | TPSKQSNN |
| | | DNSESTVYL | KALPRPIEKTISKAKGQPREPQVYTL | | SLAISGLQS | KYAASSYL |
| | | QLNSLRAED | PPSRDELTKNQVSLTCLVKGFYPSDI | | EDEADYYCA | SLTPEQWK |
| | | TAVYYCTRA | AVEWESNGQPENNYKTTPPVLDSDGS | | SWDDSLNVR | SHRSYSCQ |
| | | TLYSSYDWG | FFLYSKLTVDKSRWQQGNVFSCSVMH | | VFGGGTKLT | VTHEGSTV |
| | | LGTLVTVSS | EALHNHYTQKSLSLSPG (SEQ ID | | VL (SEQ | EKTVAPTE |
| | | (SEQ ID | NO: 587) | | ID NO: | CS (SEQ |
| | | NO: 266) | | | 387) | ID NO: |
| | | | | | | 416) |
| TA236 | MGWSCII | DVQLVESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDFAMGW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSTISGS | KDTLMISRTPEVTCVVVDVSDEDGEV | 588) | LLIYGASTR | WKVDNALQ |
| | | GVITFYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | TAVYFCSEG | AVEWESNGQPENNYKTTPPVLDSDGS | | YNNWPPWTF | KHKVYACE |
| | | MSYHDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 267) | | | | ID NO: |
| | | | | | | 415) |
| TA237 | MGWSCII | DVQLVESGG | ASTKGPSVFPLAPSSKSTSGGTAALG | MGWSCII | EIVMTQSPA | RTVAAPSV |
| | LFLVATA | GVVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDFAMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSTISGS | KDTLMISRTPEVTCVVVDVSDEDGEV | 588) | LLIYGASTR | WKVDNALQ |
| | | GVVTFYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNALYL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | TAVYFCSEG | AVEWESNGQPENNYKTTPPVLDSDGS | | YNNWPPWTF | KHKVYACE |
| | | MSYHDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 268) | | | | ID NO: |
| | | | | | | 415) |
| TA238 | MGWSCII | DVQLVESGG | ASTKGPSVFPLAPSSKSTSGGTAALG | MGWSCII | EIVMTQSPA | RTVAAPSV |
| | LFLVATA | GLVQPGGFL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFRDFAMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSSISHS | KDTLMISRTPEVTCVVVDVSDEDGEV | 588) | LLIYGASTR | WKVDNALQ |
| | | GVITFYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | TAIYYCSEG | AVEWESNGQPENNYKTTPPVLDSDGS | | YNNWPPWTF | KHKVYACE |
| | | LSYHDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 269) | | | | ID NO: |
| | | | | | | 415) |
| TA239 | MGWSCII | DVQLVESGG | ASTKGPSVFPLAPSSKSTSGGTAALG | MGWSCII | EIVMTQSPA | RTVAAPSV |
| | LFLVATA | GVVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFRDFAMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSSISHS | KDTLMISRTPEVTCVVVDVSDEDGEV | 588) | LLIYGASTR | WKVDNALQ |
| | | GVITFYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | TAVYFCSEG | AVEWESNGQPENNYKTTPPVLDSDGS | | YNNWPPWTF | KHKVYACE |
| | | LSYHDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 270) | | | | ID NO: |
| | | | | | | 415) |
| TA240 | MGWSCII | DVQLVESGG | ASTKGPSVFPLAPSSKSTSGGTAALG | MGWSCII | EIVMTQSPA | RTVAAPSV |
| | LFLVATA | GVVRPGESL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSSDAMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSAISGS | KDTLMISRTPEVTCVVVDVSDEDGEV | 588) | LLIYGASTR | WKVDNALQ |
| | | GVITYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSNNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAIYYCATG | AVEWESNGQPENNYKTTPPVLDSDGS | | YNNWPPWTF | KHKVYACE |
| | | FPFFDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 271) | | | | ID NO: |
| | | | | | | 415) |
| TA241 | MGWSCII | DVQLVESGG | ASTKGPSVFPLAPSSKSTSGGTAALG | MGWSCII | EIVMTQSPA | RTVAAPSV |
| | LFLVATA | GVVRPGESL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | SFSSYDMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSSISGS | KDTLMISRTPEVTCVVVDVSDEDGEV | 588) | LLIYGASTR | WKVDNALQ |
| | | GAITYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCAES | AVEWESNGQPENNYKTTPPVLDSDGS | | YNNWPPWTF | KHKVYACE |
| | | MIFFDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 272) | | | | ID NO: |
| | | | | | | 415) |
| TA242 | MGWSCII | DVQLVESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDFAMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSTISGS | KDTLMISRTPEVTCVVVDVSDEDGEV | 588) | LLIYGASTR | WKVDNALQ |
| | | GVIIFYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | TAVYFCSEG | AVEWESNGQPENNYKTTPPVLDSDGS | | YNNWPPWTF | KHKVYACE |
| | | MSYHDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 273) | | | | ID NO: |
| | | | | | | 415) |
| TA243 | MGWSCII | DVQLVESGG | ASTKGPSVFPLAPSSKSTSGGTAALG | MGWSCII | EIVMTQSPA | RTVAAPSV |
| | LFLVATA | GVVRPGESL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | SFDIYDMSW | QTYICNVNHKPSNTKVDKKVEPKSCD | (SEQ ID | SVSSNLAWY | VVCLLNNF |
| | NO: | ARQAPGKGL | KTHTCPPCPAPDLLGDDSVFLFPPKP | NO: | QQKPGQAPR | YPREAKVQ |
| | 588) | EWVSLISSS | KDTLMISRTPEVTCVVVDVSDEDGEV | 588) | LLIYGASTR | WKVDNALQ |
| | | GVITYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKSTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCARA | AVEWESNGQPENNYKTTPPVLDSDGS | | YNNWPPWTF | KHKVYACE |
| | | GNTFFHYWG | FFLYSKLTVDKSRWQQGNVFSCSVMH | | GQGTKVEIK | VTHQGLSS |
| | | LGTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 274) | | | | ID NO: |
| | | | | | | 415) |
| TA244 | MGWSCII | DVQLVESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSSYAMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSSISHS | KDTLMISRTPEVTCVVVDVSDEDGEV | 588) | LLIYGASTR | WKVDNALQ |
| | | GVITFYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | TAVYYCSEG | AVEWESNGQPENNYKTTPPVLDSDGS | | YNNWPPWTF | KHKVYACE |
| | | LSYHDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 275) | | | | ID NO: |
| | | | | | | 415) |
| TA245 | MGWSCII | DVQLVESGG | ASTKGPSVFPLAPSSKSTSGGTAALG | MGWSCII | EIVMTQSPA | RTVAAPSV |
| | LFLVATA | GVVRPGESL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | SFDIYDMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSLISSS | KDTLMISRTPEVTCVVVDVSDEDGEV | 588) | LLIYGASTR | WKVDNALQ |
| | | GVITYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKSTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAIYYCARA | AVEWESNGQPENNYKTTPPVLDSDGS | | YNNWPPWTF | KHKVYACE |
| | | GNTFFHYWG | FFLYSKLTVDKSRWQQGNVFSCSVMH | | GQGTKVEIK | VTHQGLSS |
| | | LGTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 276) | | | | ID NO: |
| | | | | | | 415) |
| TA246 | MGWSCII | DVQLVESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDFAMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSSISHS | KDTLMISRTPEVTCVVVDVSDEDGEV | 588) | LLIYGASTR | WKVDNALQ |
| | | GVITFYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | TAIYFCSEG | AVEWESNGQPENNYKTTPPVLDSDGS | | YNNWPPWTF | KHKVYACE |
| | | LSYHDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 277) | | | | ID NO: |
| | | | | | | 415) |
| TA247 | MGWSCII | DVQLVESGG | ASTKGPSVFPLAPSSKSTSGGTAALG | MGWSCII | EIVMTQSPA | RTVAAPSV |
| | LFLVATA | GVVRPGESL | CLVKDYFPEPVTVSWNSGALTSGVHT | LFLVATA | TLSVSPGER | FIFPPSDE |
| | TGVHS | RLSCVASGF | FPAVLQSSGLYSLSSVVTVPSSSLGT | TGVHS | ATLSCRASQ | QLKSGTAS |
| | (SEQ ID | TFSTDTMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSASSGS | KDTLMISRTPEVTCVVVDVSDEDGEV | 588) | LLIYGASTR | WKVDNALQ |
| | | GVITYYADS | KFNWYVDGVEVHNAKTKPREEQYNST | | ATGIPARFS | SGNSQESV |
| | | AKGRFTISR | YRVVSVLTVLHQDWLNGKEYKCKVSN | | GSGSGTEFT | TEQDSKDS |
| | | DNSNNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAIYYCATG | AVEWESNGQPENNYKTTPPVLDSDGS | | YNNWPPWTF | KHKVYACE |
| | | FPFFDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 278) | | | | ID NO: |
| | | | | | | 415) |
| TA248 | MGWSCII | DVQLVESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDFAMSW | QTYICNVNHKPSNTKVDKKVEPKSCD | (SEQ ID | SVSSNLAWY | VVCLLNNF |
| | NO: | IRQAPGEGL | KTHTCPPCPAPDLLGDDSVFLFPPKP | NO: | QQKPGQAPR | YPREAKVQ |
| | 588) | EWVSTISGS | KDTLMISRTPEVTCVVVDVSDEDGEV | 588) | LLIYGASTR | WKVDNALQ |
| | | GVITFYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | TAVYFCSEG | AVEWESNGQPENNYKTTPPVLDSDGS | | YNNWPPWTF | KHKVYACE |
| | | MSYHDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 279) | | | | ID NO: |
| | | | | | | 415) |
| TA249 | MGWSCII | DVQLVESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDFAVSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSTISGS | KDTLMISRTPEVTCVVVDVSDEDGEV | 588) | LLIYGASTR | WKVDNALQ |
| | | GVITFYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | TAVYFCSEG | AVEWESNGQPENNYKTTPPVLDSDGS | | YNNWPPWTF | KHKVYACE |
| | | MSYHDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 280) | | | | ID NO: |
| | | | | | | 415) |
| TA250 | MGWSCII | DVQLVESGG | ASTKGPSVFPLAPSSKSTSGGTAALG | MGWSCII | EIVMTQSPA | RTVAAPSV |
| | LFLVATA | GVVRPGESP | CLVKDYFPEPVTVSWNSGALTSGVHT | LFLVATA | TLSVSPGER | FIFPPSDE |
| | TGVHS | RLSCVASGF | FPAVLQSSGLYSLSSVVTVPSSSLGT | TGVHS | ATLSCRASQ | QLKSGTAS |
| | (SEQ ID | TFSTDTMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSASSGS | KDTLMISRTPEVTCVVVDVSDEDGEV | 588) | LLIYGASTR | WKVDNALQ |
| | | GVITYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSNNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAIYYCATG | AVEWESNGQPENNYKTTPPVLDSDGS | | YNNWPPWTF | KHKVYACE |
| | | FPFFDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 281) | | | | ID NO: |
| | | | | | | 415) |
| TA251 | MGWSCII | DVQLVESGG | ASTKGPSVFPLAPSSKSTSGGTAALG | MGWSCII | EIVMTQSPA | RTVAAPSV |
| | LFLVATA | GVVRPGESL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSSDAVSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSSISGS | KDTLMISRTPEVTCVVVDVSDEDGEV | 588) | LLIYGASTR | WKVDNALQ |
| | | GVITYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCAKD | AVEWESNGQPENNYKTTPPVLDSDGS | | YNNWPPWTF | KHKVYACE |
| | | VFFFNYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 282) | | | | ID NO: |
| | | | | | | 415) |
| TA252 | MGWSCII | DVQLVESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDFAMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSSISHS | KDTLMISRTPEVTCVVVDVSDEDGEV | 588) | LLIYGASTR | WKVDNALQ |
| | | GVITFYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | TAIYYCSEG | AVEWESNGQPENNYKTTPPVLDSDGS | | YNNWPPWTF | KHKVYACE |
| | | LSYHDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 283) | | | | ID NO: |
| | | | | | | 415) |
| TA253 | MGWSCII | DVQLVESGG | ASTKGPSVFPLAPSSKSTSGGTAALG | MGWSCII | EIVMTQSPA | RTVAAPSV |
| | LFLVATA | GVVRPGESL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSSDAMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSSISGS | KDTLMISRTPEVTCVVVDVSDEDGEV | 588) | LLIYGASTR | WKVDNALQ |
| | | GVITYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCAKD | AVEWESNGQPENNYKTTPPVLDSDGS | | YNNWPPWTF | KHKVYACE |
| | | VFFFNYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 284) | | | | ID NO: |
| | | | | | | 415) |
| TA254 | MGWSCII | DVQLVESGG | ASTKGPSVFPLAPSSKSTSGGTAALG | MGWSCII | EIVMTQSPA | RTVAAPSV |
| | LFLVATA | GVVRPGESL | CLVKDYFPEPVTVSWNSGALTSGVHT | LFLVATA | TLSVSPGER | FIFPPSDE |
| | TGVHS | RLSCVASGE | FPAVLQSSGLYSLSSVVTVPSSSLGT | TGVHS | ATLSCRASQ | QLKSGTAS |
| | (SEQ ID | TFSSDVMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSASSGS | KDTLMISRTPEVTCVVVDVSDEDGEV | 588) | LLIYGASTR | WKVDNALQ |
| | | GVITYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCAKA | AVEWESNGQPENNYKTTPPVLDSDGS | | YNNWPPWTF | KHKVYACE |
| | | GNTFLDYWG | FFLYSKLTVDKSRWQQGNVFSCSVMH | | GQGTKVEIK | VTHQGLSS |
| | | LGTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 285) | | | | ID NO: |
| | | | | | | 415) |
| TA255 | MGWSCII | DVQLVESGG | ASTKGPSVFPLAPSSKSTSGGTAALG | MGWSCII | EIVMTQSPA | RTVAAPSV |
| | LFLVATA | GVVRPGESL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | SFDIYDMSW | QTYICNVNHKPSNTKVDKKVEPKSCD | (SEQ ID | SVSSNLAWY | VVCLLNNF |
| | NO: | VRQAPGKGL | KTHTCPPCPAPDLLGDDSVFLFPPKP | NO: | QQKPGQAPR | YPREAKVQ |
| | 588) | EWVSLISSS | KDTLMISRTPEVTCVVVDVSDEDGEV | 588) | LLIYGASTR | WKVDNALQ |
| | | GVITYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKSTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAVYYCARA | AVEWESNGQPENNYKTTPPVLDSDGS | | YNNWPPWTF | KHKVYACE |
| | | GNTFFHYWG | FFLYSKLTVDKSRWQQGNVFSCSVMH | | GQGTKVEIK | VTHQGLSS |
| | | LGTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 286) | | | | ID NO: |
| | | | | | | 415) |
| TA256 | MGWSCII | DVQLVESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDFAMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSTISGS | KDTLMISRTPEVTCVVVDVSDEDGEV | 588) | LLIYGASTR | WKVDNALQ |
| | | GVITFYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNALYL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | TAVYFCSEG | AVEWESNGQPENNYKTTPPVLDSDGS | | YNNWPPWTF | KHKVYACE |
| | | MSYHDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 287) | | | | ID NO: |
| | | | | | | 415) |
| TA257 | MGWSCII | DVQLVESGG | ASTKGPSVFPLAPSSKSTSGGTAALG | MGWSCII | EIVMTQSPA | RTVAAPSV |
| | LFLVATA | GVVRPGESL | CLVKDYFPEPVTVSWNSGALTSGVHT | LFLVATA | TLSVSPGER | FIFPPSDE |
| | TGVHS | RLSCVASGF | FPAVLQSSGLYSLSSVVTVPSSSLGT | TGVHS | ATLSCRASQ | QLKSGTAS |
| | (SEQ ID | TFSTDTMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSASSGS | KDTLMISRTPEVTCVVVDVSDEDGEV | 588) | LLIYGASTR | WKVDNALQ |
| | | GVITYYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSNNTLYL | KALPRPIEKTISKAKGQPREPQVYTL | | LTISSLQSE | TYSLSSTL |
| | | QMNSLRAED | PPSRDELTKNQVSLTCLVKGFYPSDI | | DFAVYYCQQ | TLSKADYE |
| | | TAIYYCATG | AVEWESNGQPENNYKTTPPVLDSDGS | | YNNWPPWTF | KHKVYACE |
| | | FPFFDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 288) | | | | ID NO: |
| | | | | | | 415) |
| TA258 | MGWSCII | DVQLVESGG | ASTKGPSVFPLAPSSKSTSGGTAALG | MGWSCII | EIVMTQSPA | RTVAAPSV |
| | LFLVATA | GLVQPGGSL | CLVKDYFPEPVTVSWNSGALTSGVHT | LFLVATA | TLSVSPGER | FIFPPSDE |
| | TGVHS | RLSCAASGF | FPAVLQSSGLYSLSSVVTVPSSSLGT | TGVHS | ATLSCRASQ | QLKSGTAS |
| | (SEQ ID | TFSDFAMSW | QTYICNVNHKPSNTKVDKKVEPKSCD | (SEQ ID | SVSSNLAWY | VVCLLNNF |
| | NO: | VRQAPGEGL | KTHTCPPCPAPDLLGDDSVFLFPPKP | NO: | QQKPGQAPR | YPREAKVQ |
| | 588) | EWVSTISGS | KDTLMISRTPEVTCVVVDVSDEDGEV | 588) | LLIYGASTR | WKVDNALQ |
| | | GVITFYADS | KFNWYVDGVEVHNAKTKPREEQYNST | | ATGIPARFS | SGNSQESV |
| | | VKGRFTISR | YRVVSVLTVLHQDWLNGKEYKCKVSN | | GSGSGTEFT | TEQDSKDS |
| | | DNSKNTLYL | KALPRPIEKTISKAKGQPREPQVYTL | | LTISSLQSE | TYSLSSTL |
| | | QMNSLRVED | PPSRDELTKNQVSLTCLVKGFYPSDI | | DFAVYYCQQ | TLSKADYE |
| | | TAVYFCSEG | AVEWESNGQPENNYKTTPPVLDSDGS | | YNNWPPWTF | KHKVYACE |
| | | MSYHDYWGL | FFLYSKLTVDKSRWQQGNVFSCSVMH | | GQGTKVEIK | VTHQGLSS |
| | | GTLVTVSS | EALHNHYTQKSLSLSPG (SEQ ID | | (SEQ ID | PVTKSFNR |
| | | (SEQ ID | NO: 587) | | NO: 323) | GEC (SEQ |
| | | NO: 289) | | | | ID NO: |
| | | | | | | 415) |
| TA259 | MGWSCII | QVTLKESGP | ASTKGPSVFPLAPSSKSTSGGTAALG | MGWSCII | QAVVTQESA | RTVAAPSV |
| | LFLVATA | GILQPSQTL | CLVKDYFPEPVTVSWNSGALTSGVHT | LFLVATA | LTTSPGETV | FIFPPSDE |
| | TGVHS | SLTCSFSGF | FPAVLQSSGLYSLSSVVTVPSSSLGT | TGVHS | TLTCRSSTG | QLKSGTAS |
| | (SEQ ID | SLSTFGMGV | QTYICNVNHKPSNTKVDKKVEPKSCD | (SEQ ID | AVTTSNYAN | VVCLLNNF |
| | NO: | GWIRQPSGK | KTHTCPPCPAPDLLGDDSVFLFPPKP | NO: | WVQEKPDHL | YPREAKVQ |
| | 588) | GLEWLAHIW | KDTLMISRTPEVTCVVVDVSDEDGEV | 588) | FTGLIGGTN | WKVDNALQ |
| | | WDDDKYYNP | KFNWYVDGVEVHNAKTKPREEQYNST | | NRAPGVPAR | SGNSQESV |
| | | ALKSRLTIS | YRVVSVLTVLHQDWLNGKEYKCKVSN | | FSGSLIGDK | TEQDSKDS |
| | | KDTSKNQVF | KALPRPIEKTISKAKGQPREPQVYTL | | AALTITGAQ | TYSLSSTL |
| | | LKIANVDTA | PPSRDELTKNQVSLTCLVKGFYPSDI | | TEDEAIYFC | TLSKADYE |
| | | DTATYYCAR | AVEWESNGQPENNYKTTPPVLDSDGS | | ALWYSNHFI | KHKVYACE |
| | | IITTAWYFD | FFLYSKLTVDKSRWQQGNVFSCSVMH | | FGSGTKVTV | VTHQGLSS |
| | | VWGTGTTVT | EALHNHYTQKSLSLSPG (SEQ ID | | L (SEQ ID | PVTKSFNR |
| | | VSS (SEQ | NO: 587) | | NO: 388) | GEC (SEQ |
| | | ID NO: | | | | ID NO: |
| | | 290) | | | | 415) |
| TA260 | MGWSCII | QIQLVQSGP | ASTKGPSVFPLAPSSKSTSGGTAALG | MGWSCII | DIQMTQSPA | RTVAAPSV |
| | LFLVATA | ELKKPGETV | CLVKDYFPEPVTVSWNSGALTSGVHT | LFLVATA | SLSASVGET | FIFPPSDE |
| | TGVHS | KISCKASGY | FPAVLQSSGLYSLSSVVTVPSSSLGT | TGVHS | VTITCRASE | QLKSGTAS |
| | (SEQ ID | TFTTYGMSW | QTYICNVNHKPSNTKVDKKVEPKSCD | (SEQ ID | NIYSYLAWY | VVCLLNNF |
| | NO: | VKQAPGKGL | KTHTCPPCPAPDLLGDDSVFLFPPKP | NO: | QQKQGKSPQ | YPREAKVQ |
| | 588) | KWMGWINTY | KDTLMISRTPEVTCVVVDVSDEDGEV | 588) | LLVYNAKTL | WKVDNALQ |
| | | SGVPTYADD | KFNWYVDGVEVHNAKTKPREEQYNST | | AEGVPSRFS | SGNSQESV |
| | | FKGRFAFSL | YRVVSVLTVLHQDWLNGKEYKCKVSN | | GSGSGTQFS | TEQDSKDS |
| | | ETSASTAYL | KALPRPIEKTISKAKGQPREPQVYTL | | LKINSLQPE | TYSLSSTL |
| | | QINNLKNED | PPSRDELTKNQVSLTCLVKGFYPSDI | | DEGSYYCQH | TLSKADYE |
| | | TATYFCARP | AVEWESNGQPENNYKTTPPVLDSDGS | | HYGTPFTFG | KHKVYACE |
| | | RRQVFDYWG | FFLYSKLTVDKSRWQQGNVFSCSVMH | | SGTKLEIK | VTHQGLSS |
| | | QGTTLTVSS | EALHNHYTQKSLSLSPG (SEQ ID | | (SEQ ID | PVTKSFNR |
| | | (SEQ ID | NO: 587) | | NO: 389) | GEC (SEQ |
| | | NO: 291) | | | | ID NO: |
| | | | | | | 415) |
| TA261 | MGWSCII | QVKLQQSGA | ASTKGPSVFPLAPSSKSTSGGTAALG | MGWSCII | DIVLTQSPA | RTVAAPSV |
| | LFLVATA | ELVRPGASV | CLVKDYFPEPVTVSWNSGALTSGVHT | LFLVATA | SLAVSLGQR | FIFPPSDE |
| | TGVHS | KLSCKASGY | FPAVLQSSGLYSLSSVVTVPSSSLGT | TGVHS | ATISCRASK | QLKSGTAS |
| | (SEQ ID | TFTDYYINW | QTYICNVNHKPSNTKVDKKVEPKSCD | (SEQ ID | SVSTSGYSY | VVCLLNNF |
| | NO: | IKQRPGQGL | KTHTCPPCPAPDLLGDDSVFLFPPKP | NO: | MHWYQQKPG | YPREAKVQ |
| | 588) | EWIARIYPV | KDTLMISRTPEVTCVVVDVSDEDGEV | 588) | QPPKLLIYL | WKVDNALQ |
| | | SGSTYYNDK | KFNWYVDGVEVHNAKTKPREEQYNST | | ASNLESGVP | SGNSQESV |
| | | FKGKATLTA | YRVVSVLTVLHQDWLNGKEYKCKVSN | | ARFSGSGSG | TEQDSKDS |
| | | EKSSSTAYM | KALPRPIEKTISKAKGQPREPQVYTL | | TDFTLNIHP | TYSLSSTL |
| | | QLSSLTSED | PPSRDELTKNQVSLTCLVKGFYPSDI | | VEEEDAATY | TLSKADYE |
| | | SAVYFCVHI | AVEWESNGQPENNYKTTPPVLDSDGS | | YCQHSRELY | KHKVYACE |
| | | YYGNHPYYF | FFLYSKLTVDKSRWQQGNVFSCSVMH | | TFGGGTKLE | VTHQGLSS |
| | | DFWGQGTTL | EALHNHYTQKSLSLSPG (SEQ ID | | IK (SEQ | PVTKSFNR |
| | | TVSS (SEQ | NO: 587) | | ID NO: | GEC (SEQ |
| | | ID NO: | | | 390) | ID NO: |
| | | 292) | | | | 415) |
| TA262 | MGWSCII | QVQLQQPGA | ASTKGPSVFPLAPSSKSTSGGTAALG | MGWSCII | QAVVTQESA | RTVAAPSV |
| | LFLVATA | ELVKPGTSV | CLVKDYFPEPVTVSWNSGALTSGVHT | LFLVATA | LTTSPGETV | FIFPPSDE |
| | TGVHS | KMSCKASGY | FPAVLQSSGLYSLSSVVTVPSSSLGT | TGVHS | TLTCRSSTG | QLKSGTAS |
| | (SEQ ID | TFITYWITW | QTYICNVNHKPSNTKVDKKVEPKSCD | (SEQ ID | AVTTSNYAN | VVCLLNNF |
| | NO: | VKQRPGHGL | KTHTCPPCPAPDLLGDDSVFLFPPKP | NO: | WVQEKPDHL | YPREAKVQ |
| | 588) | EWIGDIYPG | KDTLMISRTPEVTCVVVDVSDEDGEV | 588) | FTGLIGGTN | WKVDNALQ |
| | | SGSTNYNAK | KFNWYVDGVEVHNAKTKPREEQYNST | | NRAPGVPAR | SGNSQESV |
| | | FRSKATLTV | YRVVSVLTVLHQDWLNGKEYKCKVSN | | FSGSLIGDK | TEQDSKDS |
| | | DTSSSTAYM | KALPRPIEKTISKAKGQPREPQVYTL | | AALTITGAQ | TYSLSSTL |
| | | QFISLTSED | PPSRDELTKNQVSLTCLVKGFYPSDI | | TEDEAIYFC | TLSKADYE |
| | | SAVYYCALK | AVEWESNGQPENNYKTTPPVLDSDGS | | ALWYSNHLV | KHKVYACE |
| | | EIVPDYWGQ | FFLYSKLTVDKSRWQQGNVFSCSVMH | | FGGGTKLTV | VTHQGLSS |
| | | GTTLTVSS | EALHNHYTQKSLSLSPG (SEQ ID | | L (SEQ ID | PVTKSFNR |
| | | (SEQ ID | NO: 587) | | NO: 391) | GEC (SEQ |
| | | NO: 293) | | | | ID NO: |
| | | | | | | 415) |
| TA263 | MGWSCII | QVQLQQPGA | ASTKGPSVFPLAPSSKSTSGGTAALG | MGWSCII | DIVLTQSPA | RTVAAPSV |
| | LFLVATA | ELVKPGTSV | CLVKDYFPEPVTVSWNSGALTSGVHT | LFLVATA | SLAVSLGQR | FIFPPSDE |
| | TGVHS | KMSCKASGY | FPAVLQSSGLYSLSSVVTVPSSSLGT | TGVHS | ATISCRASQ | QLKSGTAS |
| | (SEQ ID | TFITYWITW | QTYICNVNHKPSNTKVDKKVEPKSCD | (SEQ ID | SVSTSSYSY | VVCLLNNF |
| | NO: | VKQRPGHGL | KTHTCPPCPAPDLLGDDSVFLFPPKP | NO: | MHWYQQKPG | YPREAKVQ |
| | 588) | EWIGDIYPG | KDTLMISRTPEVTCVVVDVSDEDGEV | 588) | QPPKLLIKY | WKVDNALQ |
| | | SGSTNYNAK | KFNWYVDGVEVHNAKTKPREEQYNST | | ASNLESGVP | SGNSQESV |
| | | FRSKATLTV | YRVVSVLTVLHQDWLNGKEYKCKVSN | | ARFSGSGSG | TEQDSKDS |
| | | DTSSSTAYM | KALPRPIEKTISKAKGQPREPQVYTL | | TDFTLNIHP | TYSLSSTL |
| | | QFISLTSED | PPSRDELTKNQVSLTCLVKGFYPSDI | | VEEEDTATY | TLSKADYE |
| | | SAVYYCALK | AVEWESNGQPENNYKTTPPVLDSDGS | | YCQHSWEIP | KHKVYACE |
| | | EIVPDYWGQ | FFLYSKLTVDKSRWQQGNVFSCSVMH | | FTFGSGTKL | VTHQGLSS |
| | | GTTLTVSS | EALHNHYTQKSLSLSPG (SEQ ID | | EIK (SEQ | PVTKSFNR |
| | | (SEQ ID | NO: 587) | | ID NO: | GEC (SEQ |
| | | NO: 293) | | | 392) | ID NO: |
| | | | | | | 415) |
| TA264 | MGWSCII | QVQLQQPGA | ASTKGPSVFPLAPSSKSTSGGTAALG | MGWSCII | QAVVTQESA | RTVAAPSV |
| | LFLVATA | ELVKPGASV | CLVKDYFPEPVTVSWNSGALTSGVHT | LFLVATA | LTTSPGETV | FIFPPSDE |
| | TGVHS | KLSCKASGY | FPAVLQSSGLYSLSSVVTVPSSSLGT | TGVHS | TLTCRSSTG | QLKSGTAS |
| | (SEQ ID | TFTSYWMHW | QTYICNVNHKPSNTKVDKKVEPKSCD | (SEQ ID | AVTTSNYAN | VVCLLNNF |
| | NO: | VKQRPGQGL | KTHTCPPCPAPDLLGDDSVFLFPPKP | NO: | WVQEKPDHL | YPREAKVQ |
| | 588) | EWIGMIHPN | KDTLMISRTPEVTCVVVDVSDEDGEV | 588) | FTGLIGGTN | WKVDNALQ |
| | | SGSTNYNEK | KFNWYVDGVEVHNAKTKPREEQYNST | | NRAPGVPAR | SGNSQESV |
| | | FKSKATLTV | YRVVSVLTVLHQDWLNGKEYKCKVSN | | FSGSLIGDK | TEQDSKDS |
| | | DKSSSTAYM | KALPRPIEKTISKAKGQPREPQVYTL | | AALTITGAQ | TYSLSSTL |
| | | QLSSLISED | PPSRDELTKNQVSLTCLVKGFYPSDI | | TEDEAIYFC | TLSKADYE |
| | | SAVYYCARS | AVEWESNGQPENNYKTTPPVLDSDGS | | ALWYSNHSW | KHKVYACE |
| | | RGNYVWYFD | FFLYSKLTVDKSRWQQGNVFSCSVMH | | VFGGGTKLT | VTHQGLSS |
| | | VWGTGTTVT | EALHNHYTQKSLSLSPG (SEQ ID | | VL (SEQ | PVTKSFNR |
| | | VSS (SEQ | NO: 587) | | ID NO: | GEC (SEQ |
| | | ID NO: | | | 393) | ID NO: |
| | | 294) | | | | 415) |
| TA265 | MGWSCII | QVTLKESGP | ASTKGPSVFPLAPSSKSTSGGTAALG | MGWSCII | ENVLTQSPA | RTVAAPSV |
| | LFLVATA | GILQPSQTL | CLVKDYFPEPVTVSWNSGALTSGVHT | LFLVATA | IMSASLGEK | FIFPPSDE |
| | TGVHS | SLTCSFSGF | FPAVLQSSGLYSLSSVVTVPSSSLGT | TGVHS | VTMSCRASS | QLKSGTAS |
| | (SEQ ID | SLSTFGMGV | QTYICNVNHKPSNTKVDKKVEPKSCD | (SEQ ID | SVNYMYWYQ | VVCLLNNF |
| | NO: | GWIRQPSGK | KTHTCPPCPAPDLLGDDSVFLFPPKP | NO: | QKSDASPKL | YPREAKVQ |
| | 588) | GLEWLAHIW | KDTLMISRTPEVTCVVVDVSDEDGEV | 588) | WIYYTSNLA | WKVDNALQ |
| | | WDDDKYYNP | KFNWYVDGVEVHNAKTKPREEQYNST | | PGVPARFSG | SGNSQESV |
| | | ALKSRLTIS | YRVVSVLTVLHQDWLNGKEYKCKVSN | | SGSGNSYSL | TEQDSKDS |
| | | KDTSKNQVE | KALPRPIEKTISKAKGQPREPQVYTL | | TISSMEGED | TYSLSSTL |
| | | LKIANVDTA | PPSRDELTKNQVSLTCLVKGFYPSDI | | AATYYCQQF | TLSKADYE |
| | | DTATYYCAR | AVEWESNGQPENNYKTTPPVLDSDGS | | TSSPSTFGG | KHKVYACE |
| | | MNPRNYFDY | FFLYSKLTVDKSRWQQGNVFSCSVMH | | GTKLEIK | VTHQGLSS |
| | | WGQGTTLTV | EALHNHYTQKSLSLSPG (SEQ ID | | (SEQ ID | PVTKSFNR |
| | | SS (SEQ | NO: 587) | | NO: 394) | GEC (SEQ |
| | | ID NO: | | | | ID NO: |
| | | 295) | | | | 415) |
| TA266 | MGWSCII | QVTLKESGP | ASTKGPSVFPLAPSSKSTSGGTAALG | MGWSCII | QAVVTQESA | RTVAAPSV |
| | LFLVATA | GILQPSQTL | CLVKDYFPEPVTVSWNSGALTSGVHT | LFLVATA | LTTSPGETV | FIFPPSDE |
| | TGVHS | SLTCSFSGF | FPAVLQSSGLYSLSSVVTVPSSSLGT | TGVHS | TLTCRSSTG | QLKSGTAS |
| | (SEQ ID | SLSTFGMGV | QTYICNVNHKPSNTKVDKKVEPKSCD | (SEQ ID | AVTTSNYAN | VVCLLNNF |
| | NO: | GWIRQPSGK | KTHTCPPCPAPDLLGDDSVFLFPPKP | NO: | WVQEKPDHL | YPREAKVQ |
| | 588) | GLEWLAHIW | KDTLMISRTPEVTCVVVDVSDEDGEV | 588) | FTGLIGGTN | WKVDNALQ |
| | | WDDDKYYNP | KFNWYVDGVEVHNAKTKPREEQYNST | | NRAPGVPAR | SGNSQESV |
| | | ALKSRLTIS | YRVVSVLTVLHQDWLNGKEYKCKVSN | | FSGSLIGDK | TEQDSKDS |
| | | KDTSKNQVF | KALPRPIEKTISKAKGQPREPQVYTL | | AALTITGAQ | TYSLSSTL |
| | | LKIANVDTA | PPSRDELTKNQVSLTCLVKGFYPSDI | | TEDEAIYFC | TLSKADYE |
| | | DTATYYCAR | AVEWESNGQPENNYKTTPPVLDSDGS | | ALWYSNRWV | KHKVYACE |
| | | MNPRNYFDY | FFLYSKLTVDKSRWQQGNVFSCSVMH | | FGGGTKLTV | VTHQGLSS |
| | | WGQGTTLTV | EALHNHYTQKSLSLSPG (SEQ ID | | L (SEQ ID | PVTKSFNR |
| | | SS (SEQ | NO: 587) | | NO: 395) | GEC (SEQ |
| | | ID NO: | | | | ID NO: |
| | | 295) | | | | 415) |
| TA267 | MGWSCII | QVQLQQPGA | ASTKGPSVFPLAPSSKSTSGGTAALG | MGWSCII | ENVLTQSPA | RTVAAPSV |
| | LFLVATA | ELVKPGASV | CLVKDYFPEPVTVSWNSGALTSGVHT | LFLVATA | IMSASLGEK | FIFPPSDE |
| | TGVHS | KVSCKASGY | FPAVLQSSGLYSLSSVVTVPSSSLGT | TGVHS | VTMSCRASS | QLKSGTAS |
| | (SEQ ID | TFTSYWMHW | QTYICNVNHKPSNTKVDKKVEPKSCD | (SEQ ID | SVNYMYWYQ | VVCLLNNF |
| | NO: | VKQRPGQGL | KTHTCPPCPAPDLLGDDSVFLFPPKP | NO: | QKSDASPKL | YPREAKVQ |
| | 588) | EWIGRIHPS | KDTLMISRTPEVTCVVVDVSDEDGEV | 588) | WIYYTSNLA | WKVDNALQ |
| | | DSDTNYNQK | KFNWYVDGVEVHNAKTKPREEQYNST | | PGVPARFSG | SGNSQESV |
| | | FKGKATLTV | YRVVSVLTVLHQDWLNGKEYKCKVSN | | SGSGNSYSL | TEQDSKDS |
| | | DKSSSTAYM | KALPRPIEKTISKAKGQPREPQVYTL | | TISSMEGED | TYSLSSTL |
| | | QLSSLTSED | PPSRDELTKNQVSLTCLVKGFYPSDI | | AATYYCQQF | TLSKADYE |
| | | SAVYYCAIR | AVEWESNGQPENNYKTTPPVLDSDGS | | TSSPSTFGG | KHKVYACE |
| | | DWDLDYWGQ | FFLYSKLTVDKSRWQQGNVFSCSVMH | | GTKLEIK | VTHQGLSS |
| | | GTTLTVSS | EALHNHYTQKSLSLSPG (SEQ ID | | (SEQ ID | PVTKSFNR |
| | | (SEQ ID | NO: 587) | | NO: 394) | GEC (SEQ |
| | | NO: 296) | | | | ID NO: |
| | | | | | | 415) |
| TA268 | MGWSCII | QVQLQQPGA | ASTKGPSVFPLAPSSKSTSGGTAALG | MGWSCII | QAVVTQESA | RTVAAPSV |
| | LFLVATA | ELVKPGASV | CLVKDYFPEPVTVSWNSGALTSGVHT | LFLVATA | LTTSPGETV | FIFPPSDE |
| | TGVHS | KVSCKASGY | FPAVLQSSGLYSLSSVVTVPSSSLGT | TGVHS | TLTCRSSTG | QLKSGTAS |
| | (SEQ ID | TFTSYWMHW | QTYICNVNHKPSNTKVDKKVEPKSCD | (SEQ ID | AVTTSNYAN | VVCLLNNF |
| | NO: | VKQRPGQGL | KTHTCPPCPAPDLLGDDSVFLFPPKP | NO: | WVQEKPDHL | YPREAKVQ |
| | 588) | EWIGRIHPS | KDTLMISRTPEVTCVVVDVSDEDGEV | 588) | FTGLIGGTN | WKVDNALQ |
| | | DSDTNYNQK | KFNWYVDGVEVHNAKTKPREEQYNST | | NRAPGVPAR | SGNSQESV |
| | | FKGKATLTV | YRVVSVLTVLHQDWLNGKEYKCKVSN | | FSGSLIGDK | TEQDSKDS |
| | | DKSSSTAYM | KALPRPIEKTISKAKGQPREPQVYTL | | AALTITGAQ | TYSLSSTL |
| | | QLSSLTSED | PPSRDELTKNQVSLTCLVKGFYPSDI | | TEDEAIYFC | TLSKADYE |
| | | SAVYYCAIR | AVEWESNGQPENNYKTTPPVLDSDGS | | ALWYSNRWV | KHKVYACE |
| | | DWDLDYWGQ | FFLYSKLTVDKSRWQQGNVFSCSVMH | | FGGGTKLTV | VTHQGLSS |
| | | GTTLTVSS | EALHNHYTQKSLSLSPG (SEQ ID | | L (SEQ ID | PVTKSFNR |
| | | (SEQ ID | NO: 587) | | NO: 395) | GEC (SEQ |
| | | NO: 296) | | | | ID NO: |
| | | | | | | 415) |
| TA269 | MGWSCII | EVMLVESGG | ASTKGPSVFPLAPSSKSTSGGTAALG | MGWSCII | DIQMTQSPA | RTVAAPSV |
| | LFLVATA | GLVKPGGSL | CLVKDYFPEPVTVSWNSGALTSGVHT | LFLVATA | SQSASLGES | FIFPPSDE |
| | TGVHS | KLSCAASGE | FPAVLQSSGLYSLSSVVTVPSSSLGT | TGVHS | VTITCLASQ | QLKSGTAS |
| | (SEQ ID | TFSSYLMSW | QTYICNVNHKPSNTKVDKKVEPKSCD | (SEQ ID | TIGTWLAWY | VVCLLNNF |
| | NO: | VRQTPEKRL | KTHTCPPCPAPDLLGDDSVFLFPPKP | NO: | QQKPGKSPQ | YPREAKVQ |
| | 588) | EWVASISGG | KDTLMISRTPEVTCVVVDVSDEDGEV | 588) | VLIYAATSL | WKVDNALQ |
| | | GRDTYYPDS | KFNWYVDGVEVHNAKTKPREEQYNST | | ADGVPSRES | SGNSQESV |
| | | VKGRFTISR | YRVVSVLTVLHQDWLNGKEYKCKVSN | | GSGSGTKFS | TEQDSKDS |
| | | DNAKNTLYL | KALPRPIEKTISKAKGQPREPQVYTL | | FKISSLQAE | TYSLSSTL |
| | | QMSSLRSED | PPSRDELTKNQVSLTCLVKGFYPSDI | | DFVSYYCQQ | TLSKADYE |
| | | TALYYCARH | AVEWESNGQPENNYKTTPPVLDSDGS | | LYSTPWTFG | KHKVYACE |
| | | GVGAMNYWG | FFLYSKLTVDKSRWQQGNVFSCSVMH | | GGTKLEIK | VTHQGLSS |
| | | QGTSVTVSS | EALHNHYTQKSLSLSPG (SEQ ID | | (SEQ ID | PVTKSFNR |
| | | (SEQ ID | NO: 587) | | NO: 396) | GEC (SEQ |
| | | NO: 297) | | | | ID NO: |
| | | | | | | 415) |
| TA270 | MGWSCII | QVTLKESGP | ASTKGPSVFPLAPSSKSTSGGTAALG | MGWSCII | QAVVTQESA | GQPKAAPS |
| | LFLVATA | GILQPSQTL | CLVKDYFPEPVTVSWNSGALTSGVHT | LFLVATA | LTTSPGETV | VTLFPPSS |
| | TGVHS | SLTCSFSGF | FPAVLQSSGLYSLSSVVTVPSSSLGT | TGVHS | TLTCRSSTG | EELQANKA |
| | (SEQ ID | SLSTFGMGV | QTYICNVNHKPSNTKVDKKVEPKSCD | (SEQ ID | AVTTSNYAN | TLVCLISD |
| | NO: | GWIRQPSGK | KTHTCPPCPAPDLLGDDSVFLFPPKP | NO: | WVQEKPDHL | FYPGAVTV |
| | 588) | GLEWLAHIW | KDTLMISRTPEVTCVVVDVSDEDGEV | 588) | FTGLIGGTN | AWKADSSP |
| | | WDDDKYYNP | KFNWYVDGVEVHNAKTKPREEQYNST | | NRAPGVPAR | VKAGVETT |
| | | ALKSRLTIS | YRVVSVLTVLHQDWLNGKEYKCKVSN | | FSGSLIGDK | TPSKQSNN |
| | | KDTSKNQVE | KALPRPIEKTISKAKGQPREPQVYTL | | AALTITGAQ | KYAASSYL |
| | | LKIANVDTA | PPSRDELTKNQVSLTCLVKGFYPSDI | | TEDEAIYFC | SLTPEQWK |
| | | DTATYYCAR | AVEWESNGQPENNYKTTPPVLDSDGS | | ALWYSNHFI | SHRSYSCQ |
| | | IITTAWYFD | FFLYSKLTVDKSRWQQGNVFSCSVMH | | FGSGTKVTV | VTHEGSTV |
| | | VWGTGTTVT | EALHNHYTQKSLSLSPG (SEQ ID | | L (SEQ ID | EKTVAPTE |
| | | VSS (SEQ | NO: 587) | | NO: 388) | CS (SEQ |
| | | ID NO: | | | | ID NO: |
| | | 290) | | | | 416) |
| TA271 | MGWSCII | QVQLQQPGA | ASTKGPSVFPLAPSSKSTSGGTAALG | MGWSCII | QAVVTQESA | GQPKAAPS |
| | LFLVATA | ELVKPGTSV | CLVKDYFPEPVTVSWNSGALTSGVHT | LFLVATA | LTTSPGETV | VTLFPPSS |
| | TGVHS | KMSCKASGY | FPAVLQSSGLYSLSSVVTVPSSSLGT | TGVHS | TLTCRSSTG | EELQANKA |
| | (SEQ ID | TFITYWITW | QTYICNVNHKPSNTKVDKKVEPKSCD | (SEQ ID | AVTTSNYAN | TLVCLISD |
| | NO: | VKQRPGHGL | KTHTCPPCPAPDLLGDDSVFLFPPKP | NO: | WVQEKPDHL | FYPGAVTV |
| | 588) | EWIGDIYPG | KDTLMISRTPEVTCVVVDVSDEDGEV | 588) | FTGLIGGTN | AWKADSSP |
| | | SGSTNYNAK | KFNWYVDGVEVHNAKTKPREEQYNST | | NRAPGVPAR | VKAGVETT |
| | | FRSKATLTV | YRVVSVLTVLHQDWLNGKEYKCKVSN | | FSGSLIGDK | TPSKQSNN |
| | | DTSSSTAYM | KALPRPIEKTISKAKGQPREPQVYTL | | AALTITGAQ | KYAASSYL |
| | | QFISLTSED | PPSRDELTKNQVSLTCLVKGFYPSDI | | TEDEAIYFC | SLTPEQWK |
| | | SAVYYCALK | AVEWESNGQPENNYKTTPPVLDSDGS | | ALWYSNHLV | SHRSYSCQ |
| | | EIVPDYWGQ | FFLYSKLTVDKSRWQQGNVFSCSVMH | | FGGGTKLTV | VTHEGSTV |
| | | GTTLTVSS | EALHNHYTQKSLSLSPG (SEQ ID | | L (SEQ ID | EKTVAPTE |
| | | (SEQ ID | NO: 587) | | NO: 391) | CS (SEQ |
| | | NO: 298) | | | | ID NO: |
| | | | | | | 416) |
| TA272 | MGWSCII | QVQLQQPGA | ASTKGPSVFPLAPSSKSTSGGTAALG | MGWSCII | QAVVTQESA | GQPKAAPS |
| | LFLVATA | ELVKPGASV | CLVKDYFPEPVTVSWNSGALTSGVHT | LFLVATA | LTTSPGETV | VTLFPPSS |
| | TGVHS | KLSCKASGY | FPAVLQSSGLYSLSSVVTVPSSSLGT | TGVHS | TLTCRSSTG | EELQANKA |
| | (SEQ ID | TFTSYWMHW | QTYICNVNHKPSNTKVDKKVEPKSCD | (SEQ ID | AVTTSNYAN | TLVCLISD |
| | NO: | VKQRPGQGL | KTHTCPPCPAPDLLGDDSVFLFPPKP | NO: | WVQEKPDHL | FYPGAVTV |
| | 588) | EWIGMIHPN | KDTLMISRTPEVTCVVVDVSDEDGEV | 588) | FTGLIGGTN | AWKADSSP |
| | | SGSTNYNEK | KFNWYVDGVEVHNAKTKPREEQYNST | | NRAPGVPAR | VKAGVETT |
| | | FKSKATLTV | YRVVSVLTVLHQDWLNGKEYKCKVSN | | FSGSLIGDK | TPSKQSNN |
| | | DKSSSTAYM | KALPRPIEKTISKAKGQPREPQVYTL | | AALTITGAQ | KYAASSYL |
| | | QLSSLTSED | PPSRDELTKNQVSLTCLVKGFYPSDI | | TEDEAIYFC | SLTPEQWK |
| | | SAVYYCARS | AVEWESNGQPENNYKTTPPVLDSDGS | | ALWYSNHSW | SHRSYSCQ |
| | | RGNYVWYFD | FFLYSKLTVDKSRWQQGNVFSCSVMH | | VFGGGTKLT | VTHEGSTV |
| | | VWGTGTTVT | EALHNHYTQKSLSLSPG (SEQ ID | | VL (SEQ | EKTVAPTE |
| | | VSS (SEQ | NO: 587) | | ID NO: | CS (SEQ |
| | | ID NO: | | | 393) | ID NO: |
| | | 294) | | | | 416) |
| TA273 | MGWSCII | QVTLKESGP | ASTKGPSVFPLAPSSKSTSGGTAALG | MGWSCII | QAVVIQESA | GQPKAAPS |
| | LFLVATA | GILQPSQTL | CLVKDYFPEPVTVSWNSGALTSGVHT | LFLVATA | LTTSPGETV | VTLFPPSS |
| | TGVHS | SLTCSFSGF | FPAVLQSSGLYSLSSVVTVPSSSLGT | TGVHS | TLTCRSSTG | EELQANKA |
| | (SEQ ID | SLSTFGMGV | QTYICNVNHKPSNTKVDKKVEPKSCD | (SEQ ID | AVTTSNYAN | TLVCLISD |
| | NO: | GWIRQPSGK | KTHTCPPCPAPDLLGDDSVFLFPPKP | NO: | WVQEKPDHL | FYPGAVTV |
| | 588) | GLEWLAHIW | KDTLMISRTPEVTCVVVDVSDEDGEV | 588) | FTGLIGGTN | AWKADSSP |
| | | WDDDKYYNP | KFNWYVDGVEVHNAKTKPREEQYNST | | NRAPGVPAR | VKAGVETT |
| | | ALKSRLTIS | YRVVSVLIVLHQDWLNGKEYKCKVSN | | FSGSLIGDK | TPSKQSNN |
| | | KDTSKNQVF | KALPRPIEKTISKAKGQPREPQVYTL | | AALTITGAQ | KYAASSYL |
| | | LKIANVDTA | PPSRDELTKNQVSLTCLVKGFYPSDI | | TEDEAIYFC | SLTPEQWK |
| | | DTATYYCAR | AVEWESNGQPENNYKTTPPVLDSDGS | | ALWYSNRWV | SHRSYSCQ |
| | | MNPRNYFDY | FFLYSKLTVDKSRWQQGNVFSCSVMH | | FGGGTKLTV | VTHEGSTV |
| | | WGQGTTLTV | EALHNHYTQKSLSLSPG (SEQ ID | | L (SEQ ID | EKTVAPTE |
| | | SS (SEQ | NO: 587) | | NO: 395) | CS (SEQ |
| | | ID NO: | | | | ID NO: |
| | | 295) | | | | 416) |
| TA274 | MGWSCII | QVQLQQPGA | ASTKGPSVFPLAPSSKSTSGGTAALG | MGWSCII | QAVVIQESA | GQPKAAPS |
| | LFLVATA | ELVKPGASV | CLVKDYFPEPVTVSWNSGALTSGVHT | LFLVATA | LTTSPGETV | VTLFPPSS |
| | TGVHS | KVSCKASGY | FPAVLQSSGLYSLSSVVTVPSSSLGT | TGVHS | TLTCRSSTG | EELQANKA |
| | (SEQ ID | TFTSYWMHW | QTYICNVNHKPSNTKVDKKVEPKSCD | (SEQ ID | AVTTSNYAN | TLVCLISD |
| | NO: | VKQRPGQGL | KTHTCPPCPAPDLLGDDSVFLFPPKP | NO: | WVQEKPDHL | FYPGAVTV |
| | 588) | EWIGRIHPS | KDTLMISRTPEVTCVVVDVSDEDGEV | 588) | FTGLIGGTN | AWKADSSP |
| | | DSDTNYNQK | KFNWYVDGVEVHNAKTKPREEQYNST | | NRAPGVPAR | VKAGVETT |
| | | FKGKATLTV | YRVVSVLIVLHQDWLNGKEYKCKVSN | | FSGSLIGDK | TPSKQSNN |
| | | DKSSSTAYM | KALPRPIEKTISKAKGQPREPQVYTL | | AALTITGAQ | KYAASSYL |
| | | QLSSLTSED | PPSRDELTKNQVSLTCLVKGFYPSDI | | TEDEAIYFC | SLTPEQWK |
| | | SAVYYCAIR | AVEWESNGQPENNYKTTPPVLDSDGS | | ALWYSNRWV | SHRSYSCQ |
| | | DWDLDYWGQ | FFLYSKLTVDKSRWQQGNVFSCSVMH | | FGGGTKLTV | VTHEGSTV |
| | | GTTLTVSS | EALHNHYTQKSLSLSPG (SEQ ID | | L (SEQ ID | EKTVAPTE |
| | | (SEQ ID | NO: 587) | | NO: 395) | CS (SEQ |
| | | NO: 296) | | | | ID NO: |
| | | | | | | 416) |
| TA275 | MGWSCII | QVTLKESGP | ASTKGPSVFPLAPSSKSTSGGTAALG | MGWSCII | QAVVTQESA | GQPKAAPS |
| | LFLVATA | GILQPSQTL | CLVKDYFPEPVTVSWNSGALTSGVHT | LFLVATA | LTTSPGETV | VTLFPPSS |
| | TGVHS | SLTCSFSGF | FPAVLQSSGLYSLSSVVTVPSSSLGT | TGVHS | TLTCRSSTG | EELQANKA |
| | (SEQ ID | SLSTFGMGV | QTYICNVNHKPSNTKVDKKVEPKSCD | (SEQ ID | AVTTSNYAN | TLVCLISD |
| | NO: | GWIRQPSGK | KTHTCPPCPAPDLLGDDSVFLFPPKP | NO: | WVQEKPDHL | FYPGAVTV |
| | 588) | GLEWLAHIW | KDTLMISRTPEVTCVVVDVSDEDGEV | 588) | FTGLIGGTN | AWKADSSP |
| | | WDDDKYYNP | KFNWYVDGVEVHNAKTKPREEQYNST | | NRAPGVPAR | VKAGVETT |
| | | ALKSRLTIS | YRVVSVLTVLHQDWLNGKEYKCKVSN | | FSGSLIGDK | TPSKQSNN |
| | | KDTSKNQVF | KALPRPIEKTISKAKGQPREPQVYTL | | AALTITGAQ | KYAASSYL |
| | | LKIANVDTA | PPSRDELTKNQVSLTCLVKGFYPSDI | | TEDEAIYFC | SLTPEQWK |
| | | DTATYYCAR | AVEWESNGQPENNYKTTPPVLDSDGS | | ALWYSTHYV | SHRSYSCQ |
| | | IPTRYWYFD | FFLYSKLTVDKSRWQQGNVFSCSVMH | | FGGGTKVTV | VTHEGSTV |
| | | VWGTGTTVT | EALHNHYTQKSLSLSPG (SEQ ID | | L (SEQ ID | EKTVAPTE |
| | | VSS (SEQ | NO: 587) | | NO: 397) | CS (SEQ |
| | | ID NO: | | | | ID NO: |
| | | 299) | | | | 416) |
| TA276 | MGWSCII | QAYLQQSGA | ASTKGPSVFPLAPSSKSTSGGTAALG | MGWSCII | QAVVTQESA | GQPKAAPS |
| | LFLVATA | ELVRPGASV | CLVKDYFPEPVTVSWNSGALTSGVHT | LFLVATA | LTTSPGETV | VTLFPPSS |
| | TGVHS | KMSCKASGY | FPAVLQSSGLYSLSSVVTVPSSSLGT | TGVHS | TLTCRSSTG | EELQANKA |
| | (SEQ ID | TFTSYNMHW | QTYICNVNHKPSNTKVDKKVEPKSCD | (SEQ ID | AVTTSNYAN | TLVCLISD |
| | NO: | VKQTPRQGL | KTHTCPPCPAPDLLGDDSVFLFPPKP | NO: | WVQEKPDHL | FYPGAVTV |
| | 588) | EWIGAIYPG | KDTLMISRTPEVTCVVVDVSDEDGEV | 588) | FTGLIGGTN | AWKADSSP |
| | | NGDTSYNQK | KFNWYVDGVEVHNAKTKPREEQYNST | | NRAPGVPAR | VKAGVETT |
| | | FKGKATLTV | YRVVSVLTVLHQDWLNGKEYKCKVSN | | FSGSLIGDK | TPSKQSNN |
| | | DKSSSTAYM | KALPRPIEKTISKAKGQPREPQVYTL | | AALTITGAQ | KYAASSYL |
| | | QLSSLTSED | PPSRDELTKNQVSLTCLVKGFYPSDI | | TEDEAIYFC | SLTPEQWK |
| | | SAVYFCARS | AVEWESNGQPENNYKTTPPVLDSDGS | | ALWYSTHYV | SHRSYSCQ |
| | | RDYDGLYAM | FFLYSKLTVDKSRWQQGNVFSCSVMH | | FGGGTKVTV | VTHEGSTV |
| | | DYWGQGTSV | EALHNHYTQKSLSLSPG (SEQ ID | | L (SEQ ID | EKTVAPTE |
| | | TVSS (SEQ | NO: 587) | | NO: 397) | CS (SEQ |
| | | ID NO: | | | | ID NO: |
| | | 300) | | | | 416) |
| TA277 | MGWSCII | QVTLKESGP | ASTKGPSVFPLAPSSKSTSGGTAALG | MGWSCII | QAVVTQESA | GQPKAAPS |
| | LFLVATA | GILQPSQTL | CLVKDYFPEPVTVSWNSGALTSGVHT | LFLVATA | LTTSPGGTV | VTLFPPSS |
| | TGVHS | SLTCSFSGF | FPAVLQSSGLYSLSSVVTVPSSSLGT | TGVHS | ILTCRSSTG | EELQANKA |
| | (SEQ ID | SLSTFGMGV | QTYICNVNHKPSNTKVDKKVEPKSCD | (SEQ ID | AVTTSNYAN | TLVCLISD |
| | NO: | GWIRQPSGK | KTHTCPPCPAPDLLGDDSVFLFPPKP | NO: | WVQEKPDHL | FYPGAVTV |
| | 588) | GLEWLAHIW | KDTLMISRTPEVTCVVVDVSDEDGEV | 588) | FTGLIGGTS | AWKADSSP |
| | | WDDDKYYNP | KFNWYVDGVEVHNAKTKPREEQYNST | | NRAPGVPVR | VKAGVETT |
| | | ALKSRLTIS | YRVVSVLTVLHQDWLNGKEYKCKVSN | | FSGSLIGDK | TPSKQSNN |
| | | KDTSKNQVF | KALPRPIEKTISKAKGQPREPQVYTL | | AALTITGAQ | KYAASSYL |
| | | LKIANVDTA | PPSRDELTKNQVSLTCLVKGFYPSDI | | TEDDAMYFC | SLTPEQWK |
| | | DTATYYCAR | AVEWESNGQPENNYKTTPPVLDSDGS | | ALWYSTHYV | SHRSYSCQ |
| | | KWNYWYFDV | FFLYSKLTVDKSRWQQGNVFSCSVMH | | FGGGTKVTV | VTHEGSTV |
| | | WGTGTTVTV | EALHNHYTQKSLSLSPG (SEQ ID | | L (SEQ ID | EKTVAPTE |
| | | SS (SEQ | NO: 587) | | NO: 398) | CS (SEQ |
| | | ID NO: | | | | ID NO: |
| | | 301) | | | | 416) |
| TA278 | MGWSCII | QVTLKESGP | ASTKGPSVFPLAPSSKSTSGGTAALG | MGWSCII | DIQMTQSPA | RTVAAPSV |
| | LFLVATA | GILQPSQTL | CLVKDYFPEPVTVSWNSGALTSGVHT | LFLVATA | SLSASVGET | FIFPPSDE |
| | TGVHS | SLTCSFSGF | FPAVLQSSGLYSLSSVVTVPSSSLGT | TGVHS | VTITCGASE | QLKSGTAS |
| | (SEQ ID | SLSTFGMGV | QTYICNVNHKPSNTKVDKKVEPKSCD | (SEQ ID | NIYGGLNWY | VVCLLNNF |
| | NO: | GWIRQPSGK | KTHTCPPCPAPDLLGDDSVFLFPPKP | NO: | QRKQGKSPQ | YPREAKVQ |
| | 588) | GLEWLAHIW | KDTLMISRTPEVTCVVVDVSDEDGEV | 588) | LLIYGATNL | WKVDNALQ |
| | | WDDDKYYNP | KFNWYVDGVEVHNAKTKPREEQYNST | | ADGMSSRFS | SGNSQESV |
| | | ALKSRLTIS | YRVVSVLTVLHQDWLNGKEYKCKVSN | | GSGSGRQYS | TEQDSKDS |
| | | KDTSKNQVF | KALPRPIEKTISKAKGQPREPQVYTL | | LKIRSLHPD | TYSLSSTL |
| | | LKIANVDTA | PPSRDELTKNQVSLTCLVKGFYPSDI | | DVATYYCQN | TLSKADYE |
| | | DTATYYCAR | AVEWESNGQPENNYKTTPPVLDSDGS | | VLSIPYTFG | KHKVYACE |
| | | KWNYWYFDV | FFLYSKLTVDKSRWQQGNVFSCSVMH | | GGTKLEIK | VTHQGLSS |
| | | WGTGTTVTV | EALHNHYTQKSLSLSPG (SEQ ID | | (SEQ ID | PVTKSFNR |
| | | SS (SEQ | NO: 587) | | NO: 399) | GEC (SEQ |
| | | ID NO: | | | | ID NO: |
| | | 301) | | | | 415) |
| TA279 | MGWSCII | QVQLQQPGA | ASTKGPSVFPLAPSSKSTSGGTAALG | MGWSCII | QAVVTQESA | GQPKAAPS |
| | LFLVATA | ELVKPGASV | CLVKDYFPEPVTVSWNSGALTSGVHT | LFLVATA | LTTSPGGTV | VTLFPPSS |
| | TGVHS | KMSCKASGD | FPAVLQSSGLYSLSSVVTVPSSSLGT | TGVHS | ILTCRSSTG | EELQANKA |
| | (SEQ ID | TFTSYWITW | QTYICNVNHKPSNTKVDKKVEPKSCD | (SEQ ID | AVTTSNYAN | TLVCLISD |
| | NO: | VKQRPGQGL | KTHTCPPCPAPDLLGDDSVFLFPPKP | NO: | WVQEKPDHL | FYPGAVTV |
| | 588) | EWIGDIYPG | KDTLMISRTPEVTCVVVDVSDEDGEV | 588) | FTGLIGGTS | AWKADSSP |
| | | SGSTNYNEK | KFNWYVDGVEVHNAKTKPREEQYNST | | NRAPGVPVR | VKAGVETT |
| | | FKSKATLTV | YRVVSVLTVLHQDWLNGKEYKCKVSN | | FSGSLIGDK | TPSKQSNN |
| | | DTSSSTAYM | KALPRPIEKTISKAKGQPREPQVYTL | | AALTITGAQ | KYAASSYL |
| | | QLSSLTSED | PPSRDELTKNQVSLTCLVKGFYPSDI | | TEDDAMYFC | SLTPEQWK |
| | | SAVYYCARK | AVEWESNGQPENNYKTTPPVLDSDGS | | ALWYSTHYV | SHRSYSCQ |
| | | WNYWYFDVW | FFLYSKLTVDKSRWQQGNVFSCSVMH | | FGGGTKVTV | VTHEGSTV |
| | | GTGTTVTVS | EALHNHYTQKSLSLSPG (SEQ ID | | L (SEQ ID | EKTVAPTE |
| | | S (SEQ ID | NO: 587) | | NO: 398) | CS (SEQ |
| | | NO: 302) | | | | ID NO: |
| | | | | | | 416) |
| TA280 | MGWSCII | QVQLQQPGA | ASTKGPSVFPLAPSSKSTSGGTAALG | MGWSCII | DIQMTQSPA | RTVAAPSV |
| | LFLVATA | ELVKPGASV | CLVKDYFPEPVTVSWNSGALTSGVHT | LFLVATA | SLSASVGET | FIFPPSDE |
| | TGVHS | KMSCKASGD | FPAVLQSSGLYSLSSVVTVPSSSLGT | TGVHS | VTITCGASE | QLKSGTAS |
| | (SEQ ID | TFTSYWITW | QTYICNVNHKPSNTKVDKKVEPKSCD | (SEQ ID | NIYGGLNWY | VVCLLNNF |
| | NO: | VKQRPGQGL | KTHTCPPCPAPDLLGDDSVFLFPPKP | NO: | QRKQGKSPQ | YPREAKVQ |
| | 588) | EWIGDIYPG | KDTLMISRTPEVTCVVVDVSDEDGEV | 588) | LLIYGATNL | WKVDNALQ |
| | | SGSTNYNEK | KFNWYVDGVEVHNAKTKPREEQYNST | | ADGMSSRFS | SGNSQESV |
| | | FKSKATLTV | YRVVSVLTVLHQDWLNGKEYKCKVSN | | GSGSGRQYS | TEQDSKDS |
| | | DTSSSTAYM | KALPRPIEKTISKAKGQPREPQVYTL | | LKIRSLHPD | TYSLSSTL |
| | | QLSSLTSED | PPSRDELTKNQVSLTCLVKGFYPSDI | | DVATYYCQN | TLSKADYE |
| | | SAVYYCARK | AVEWESNGQPENNYKTTPPVLDSDGS | | VLSIPYTFG | KHKVYACE |
| | | WNYWYFDVW | FFLYSKLTVDKSRWQQGNVFSCSVMH | | GGTKLEIK | VTHQGLSS |
| | | GTGTTVTVS | EALHNHYTQKSLSLSPG (SEQ ID | | (SEQ ID | PVTKSFNR |
| | | S (SEQ ID | NO: 587) | | NO: 399) | GEC (SEQ |
| | | NO: 302) | | | | ID NO: |
| | | | | | | 415) |
| TA281 | MGWSCII | QVQLQQPGA | ASTKGPSVFPLAPSSKSTSGGTAALG | MGWSCII | QAVVTQESA | GQPKAAPS |
| | LFLVATA | ELVKPGASV | CLVKDYFPEPVTVSWNSGALTSGVHT | LFLVATA | LTTSPGGTV | VTLFPPSS |
| | TGVHS | KMSCKASGD | FPAVLQSSGLYSLSSVVTVPSSSLGT | TGVHS | ILTCRSSTG | EELQANKA |
| | (SEQ ID | TFTSYWITW | QTYICNVNHKPSNTKVDKKVEPKSCD | (SEQ ID | AVTTSNYAN | TLVCLISD |
| | NO: | VKQRPGQGL | KTHTCPPCPAPDLLGDDSVFLFPPKP | NO: | WVQEKPDHL | FYPGAVTV |
| | 588) | EWIGDIYPG | KDTLMISRTPEVTCVVVDVSDEDGEV | 588) | FTGLIGGTS | AWKADSSP |
| | | SGSTNYNEK | KFNWYVDGVEVHNAKTKPREEQYNST | | NRAPGVPVR | VKAGVETT |
| | | FKSKATLTV | YRVVSVLTVLHQDWLNGKEYKCKVSN | | FSGSLIGDK | TPSKQSNN |
| | | DTSSSTAYM | KALPRPIEKTISKAKGQPREPQVYTL | | AALTITGAQ | KYAASSYL |
| | | QLSSLTSED | PPSRDELTKNQVSLTCLVKGFYPSDI | | TEDDAMYFC | SLTPEQWK |
| | | SAVYYCARR | AVEWESNGQPENNYKTTPPVLDSDGS | | ALWYSTHYV | SHRSYSCQ |
| | | YYHGSSWYF | FFLYSKLTVDKSRWQQGNVFSCSVMH | | FGGGTKVTV | VTHEGSTV |
| | | DVWGTGTTV | EALHNHYTQKSLSLSPG (SEQ ID | | L (SEQ ID | EKTVAPTE |
| | | TVSS (SEQ | NO: 587) | | NO: 398) | CS (SEQ |
| | | ID NO: | | | | ID NO: |
| | | 303) | | | | 416) |
| TA282 | MGWSCII | QVQLQQPGA | ASTKGPSVFPLAPSSKSTSGGTAALG | MGWSCII | DIQMTQSPA | RTVAAPSV |
| | LFLVATA | ELVKPGASV | CLVKDYFPEPVTVSWNSGALTSGVHT | LFLVATA | SLSASVGET | FIFPPSDE |
| | TGVHS | KMSCKASGD | FPAVLQSSGLYSLSSVVTVPSSSLGT | TGVHS | VTITCGASE | QLKSGTAS |
| | (SEQ ID | TFTSYWITW | QTYICNVNHKPSNTKVDKKVEPKSCD | (SEQ ID | NIYGGLNWY | VVCLLNNF |
| | NO: | VKQRPGQGL | KTHTCPPCPAPDLLGDDSVFLFPPKP | NO: | QRKQGKSPQ | YPREAKVQ |
| | 588) | EWIGDIYPG | KDTLMISRTPEVTCVVVDVSDEDGEV | 588) | LLIYGATNL | WKVDNALQ |
| | | SGSTNYNEK | KFNWYVDGVEVHNAKTKPREEQYNST | | ADGMSSRFS | SGNSQESV |
| | | FKSKATLTV | YRVVSVLTVLHQDWLNGKEYKCKVSN | | GSGSGRQYS | TEQDSKDS |
| | | DTSSSTAYM | KALPRPIEKTISKAKGQPREPQVYTL | | LKIRSLHPD | TYSLSSTL |
| | | QLSSLTSED | PPSRDELTKNQVSLTCLVKGFYPSDI | | DVATYYCQN | TLSKADYE |
| | | SAVYYCARR | AVEWESNGQPENNYKTTPPVLDSDGS | | VLSIPYTFG | KHKVYACE |
| | | YYHGSSWYF | FFLYSKLTVDKSRWQQGNVFSCSVMH | | GGTKLEIK | VTHQGLSS |
| | | DVWGTGTTV | EALHNHYTQKSLSLSPG (SEQ ID | | (SEQ ID | PVTKSFNR |
| | | TVSS (SEQ | NO: 587) | | NO: 399) | GEC (SEQ |
| | | ID NO: | | | | ID NO: |
| | | 303) | | | | 415) |
| TA283 | MGWSCII | QIQFVQSGP | ASTKGPSVFPLAPSSKSTSGGTAALG | MGWSCII | DIVLTQSPA | RTVAAPSV |
| | LFLVATA | ELKKPGETV | CLVKDYFPEPVTVSWNSGALTSGVHT | LFLVATA | SLAVSLGQR | FIFPPSDE |
| | TGVHS | KISCKASVY | FPAVLQSSGLYSLSSVVTVPSSSLGT | TGVHS | ATISCRASE | QLKSGTAS |
| | (SEQ ID | TFTEYPIHW | QTYICNVNHKPSNTKVDKKVEPKSCD | (SEQ ID | SVDNYGISF | VVCLLNNF |
| | NO: | VKQAPGKGF | KTHTCPPCPAPDLLGDDSVFLFPPKP | NO: | MKWFQQKPG | YPREAKVQ |
| | 588) | KWMGCINTY | KDTLMISRTPEVTCVVVDVSDEDGEV | 588) | QSPKLLIYA | WKVDNALQ |
| | | SGEPTYADD | KFNWYVDGVEVHNAKTKPREEQYNST | | ASNQGSGVP | SGNSQESV |
| | | FKRRFAFSL | YRVVSVLTVLHQDWLNGKEYKCKVSN | | ARFSGSGSG | TEQDSKDS |
| | | ETSASTAYL | KALPRPIEKTISKAKGQPREPQVYTL | | TDFSLNIHP | TYSLSSTL |
| | | QINNLKNED | PPSRDELTKNQVSLTCLVKGFYPSDI | | MEEDDTAMY | TLSKADYE |
| | | TATYFCARC | AVEWESNGQPENNYKTTPPVLDSDGS | | FCQQSKEVP | KHKVYACE |
| | | YGYDVFDYW | FFLYSKLTVDKSRWQQGNVFSCSVMH | | RTFGGGTKL | VTHQGLSS |
| | | GQGTTLTVS | EALHNHYTQKSLSLSPG (SEQ ID | | EVK (SEQ | PVTKSFNR |
| | | S (SEQ ID | NO: 587) | | ID NO: | GEC (SEQ |
| | | NO: 304) | | | 400) | ID NO: |
| | | | | | | 415) |
| TA284 | MGWSCII | QVQLQQSGA | ASTKGPSVFPLAPSSKSTSGGTAALG | MGWSCII | DIVLTQSPA | RTVAAPSV |
| | LFLVATA | ELVRPGASV | CLVKDYFPEPVTVSWNSGALTSGVHT | LFLVATA | SLAVSLGQR | FIFPPSDE |
| | TGVHS | TLSCKASGY | FPAVLQSSGLYSLSSVVTVPSSSLGT | TGVHS | ATISCRASE | QLKSGTAS |
| | (SEQ ID | TFTDYEMHW | QTYICNVNHKPSNTKVDKKVEPKSCD | (SEQ ID | SVDNYGISF | VVCLLNNF |
| | NO: | VKQTPVHGL | KTHTCPPCPAPDLLGDDSVFLFPPKP | NO: | MKWFQQKPG | YPREAKVQ |
| | 588) | EWIGTIDPE | KDTLMISRTPEVTCVVVDVSDEDGEV | 588) | QSPKLLIYA | WKVDNALQ |
| | | TGGTAYNQK | KFNWYVDGVEVHNAKTKPREEQYNST | | ASNQGSGVP | SGNSQESV |
| | | FKGKAILTA | YRVVSVLTVLHQDWLNGKEYKCKVSN | | ARFSGSGSG | TEQDSKDS |
| | | DKSSSTAYM | KALPRPIEKTISKAKGQPREPQVYTL | | TDFSLNIHP | TYSLSSTL |
| | | VLRSLTSED | PPSRDELTKNQVSLTCLVKGFYPSDI | | MEEDDTAMY | TLSKADYE |
| | | SAVYYCTRE | AVEWESNGQPENNYKTTPPVLDSDGS | | FCQQSKEVP | KHKVYACE |
| | | GYGSPYYFD | FFLYSKLTVDKSRWQQGNVFSCSVMH | | RTFGGGTKL | VTHQGLSS |
| | | YWGQGTTLT | EALHNHYTQKSLSLSPG (SEQ ID | | EVK (SEQ | PVTKSFNR |
| | | VSS (SEQ | NO: 587) | | ID NO: | GEC (SEQ |
| | | ID NO: | | | 400) | ID NO: |
| | | 305) | | | | 415) |
| TA285 | MGWSCII | QVTLKESGP | ASTKGPSVFPLAPSSKSTSGGTAALG | MGWSCII | DIVMTQSHK | RTVAAPSV |
| | LFLVATA | GILQPSQTL | CLVKDYFPEPVTVSWNSGALTSGVHT | LFLVATA | FMSTSVGDR | FIFPPSDE |
| | TGVHS | SLTCSFSGF | FPAVLQSSGLYSLSSVVTVPSSSLGT | TGVHS | VSITCKASQ | QLKSGTAS |
| | (SEQ ID | SLSTFGMGV | QTYICNVNHKPSNTKVDKKVEPKSCD | (SEQ ID | DVSTAVAWY | VVCLLNNF |
| | NO: | GWIRQPSGK | KTHTCPPCPAPDLLGDDSVFLFPPKP | NO: | QQKPGQSPK | YPREAKVQ |
| | 588) | GLEWLAHIW | KDTLMISRTPEVTCVVVDVSDEDGEV | 588) | LLIYSASYR | WKVDNALQ |
| | | WDDDKYYNP | KFNWYVDGVEVHNAKTKPREEQYNST | | YTGVPDRFT | SGNSQESV |
| | | ALKSRLTIS | YRVVSVLTVLHQDWLNGKEYKCKVSN | | GSGSGTDFT | TEQDSKDS |
| | | KDTSKNQVF | KALPRPIEKTISKAKGQPREPQVYTL | | FTISSVQAE | TYSLSSTL |
| | | LKIANVDTA | PPSRDELTKNQVSLTCLVKGFYPSDI | | DLAVYYCQQ | TLSKADYE |
| | | DTATYYCAR | AVEWESNGQPENNYKTTPPVLDSDGS | | HYSTPYTFG | KHKVYACE |
| | | IAEGRWYFD | FFLYSKLTVDKSRWQQGNVFSCSVMH | | GGTKLEIK | VTHQGLSS |
| | | VWGTGTTVT | EALHNHYTQKSLSLSPG (SEQ ID | | (SEQ ID | PVTKSFNR |
| | | VSS (SEQ | NO: 587) | | NO: 401) | GEC (SEQ |
| | | ID NO: | | | | ID NO: |
| | | 306) | | | | 415) |
| TA286 | MGWSCII | QVQLQQPGA | ASTKGPSVFPLAPSSKSTSGGTAALG | MGWSCII | DIQMTQTTS | RTVAAPSV |
| | LFLVATA | ELVMPGASV | CLVKDYFPEPVTVSWNSGALTSGVHT | LFLVATA | SLSVSLGDR | FIFPPSDE |
| | TGVHS | KLSCKASGY | FPAVLQSSGLYSLSSVVTVPSSSLGT | TGVHS | VTISCSASQ | QLKSGTAS |
| | (SEQ ID | TFTSYWMHW | QTYICNVNHKPSNTKVDKKVEPKSCD | (SEQ ID | VITNYLNWY | VVCLLNNF |
| | NO: | VKQRPGQGL | KTHTCPPCPAPDLLGDDSVFLFPPKP | NO: | QQKPDGTIK | YPREAKVQ |
| | 588) | EWIGEIDPS | KDTLMISRTPEVTCVVVDVSDEDGEV | 588) | LLIYYTSSL | WKVDNALQ |
| | | DSYTNYNQK | KFNWYVDGVEVHNAKTKPREEQYNST | | HSGVPSRES | SGNSQESV |
| | | FKGKSTLTV | YRVVSVLTVLHQDWLNGKEYKCKVSN | | GSGSGTDYS | TEQDSKDS |
| | | DKSSSTAYM | KALPRPIEKTISKAKGQPREPQVYTL | | LTISNLEPE | TYSLSSTL |
| | | QLSSLTSED | PPSRDELTKNQVSLTCLVKGFYPSDI | | DIATYFCQQ | TLSKADYE |
| | | SAVYYCARS | AVEWESNGQPENNYKTTPPVLDSDGS | | YDKLPWTFG | KHKVYACE |
| | | PFDYWGQGT | FFLYSKLTVDKSRWQQGNVFSCSVMH | | GGTKLEIK | VTHQGLSS |
| | | TLTVSS | EALHNHYTQKSLSLSPG (SEQ ID | | (SEQ ID | PVTKSFNR |
| | | (SEQ ID | NO: 587) | | NO: 402) | GEC (SEQ |
| | | NO: 307) | | | | ID NO: |
| | | | | | | 415) |
| TA287 | MGWSCII | EVQLQQSGP | ASTKGPSVFPLAPSSKSTSGGTAALG | MGWSCII | DIQMTQTTS | RTVAAPSV |
| | LFLVATA | ELVKPGASV | CLVKDYFPEPVTVSWNSGALTSGVHT | LFLVATA | SLSVSLGDR | FIFPPSDE |
| | TGVHS | KIPCKASGY | FPAVLQSSGLYSLSSVVTVPSSSLGT | TGVHS | VTISCSASQ | QLKSGTAS |
| | (SEQ ID | TFTDYNMDW | QTYICNVNHKPSNTKVDKKVEPKSCD | (SEQ ID | VITNYLNWY | VVCLLNNF |
| | NO: | VKQSHGKSL | KTHTCPPCPAPDLLGDDSVFLFPPKP | NO: | QQKPDGTIK | YPREAKVQ |
| | 588) | EWIGDINPN | KDTLMISRTPEVTCVVVDVSDEDGEV | 588) | LLIYYTSSL | WKVDNALQ |
| | | NGGTIYNQK | KFNWYVDGVEVHNAKTKPREEQYNST | | HSGVPSRES | SGNSQESV |
| | | FKGKATLTV | YRVVSVLTVLHQDWLNGKEYKCKVSN | | GSGSGTDYS | TEQDSKDS |
| | | DKSSSTAYM | KALPRPIEKTISKAKGQPREPQVYTL | | LTISNLEPE | TYSLSSTL |
| | | ELRSLTSED | PPSRDELTKNQVSLTCLVKGFYPSDI | | DIATYFCQQ | TLSKADYE |
| | | TGVYYCVTS | AVEWESNGQPENNYKTTPPVLDSDGS | | YDKLPWTFG | KHKVYACE |
| | | IYYDSAWFG | FFLYSKLTVDKSRWQQGNVFSCSVMH | | GGTKLEIK | VTHQGLSS |
| | | YWGQGTLVT | EALHNHYTQKSLSLSPG (SEQ ID | | (SEQ ID | PVTKSFNR |
| | | VSA (SEQ | NO: 587) | | NO: 402) | GEC (SEQ |
| | | ID NO: | | | | ID NO: |
| | | 308) | | | | 415) |
| TA288 | MGWSCII | QIQLVQSGP | ASTKGPSVFPLAPSSKSTSGGTAALG | MGWSCII | QAVVTQESA | GQPKAAPS |
| | LFLVATA | ELKKPGETV | CLVKDYFPEPVTVSWNSGALTSGVHT | LFLVATA | LTTSPGETV | VTLFPPSS |
| | TGVHS | KISCKASGY | FPAVLQSSGLYSLSSVVTVPSSSLGT | TGVHS | TLTCRSSTG | EELQANKA |
| | (SEQ ID | TFTTYGMSW | QTYICNVNHKPSNTKVDKKVEPKSCD | (SEQ ID | AVTTSNYAN | TLVCLISD |
| | NO: | VKQAPGKGL | KTHTCPPCPAPDLLGDDSVFLFPPKP | NO: | WVQEKPDHL | FYPGAVTV |
| | 588) | KWMGWINTY | KDTLMISRTPEVTCVVVDVSDEDGEV | 588) | FTGLIGGTN | AWKADSSP |
| | | SGVPAYAAD | KFNWYVDGVEVHNAKTKPREEQYNST | | NRAPGVPAR | VKAGVETT |
| | | FKGRFAFSL | YRVVSVLTVLHQDWLNGKEYKCKVSN | | FSGSLIGDK | TPSKQSNN |
| | | ETSASTAYL | KALPRPIEKTISKAKGQPREPQVYTL | | AALTITGAQ | KYAASSYL |
| | | QINTLKNED | PPSRDELTKNQVSLTCLVKGFYPSDI | | TEDEAIYFC | SLTPEQWK |
| | | TATYFCARG | AVEWESNGQPENNYKTTPPVLDSDGS | | ALWYSNQFI | SHRSYSCQ |
| | | GNPYWGQGT | FFLYSKLTVDKSRWQQGNVFSCSVMH | | FGSGTKVTV | VTHEGSTV |
| | | LVTVSA | EALHNHYTQKSLSLSPG (SEQ ID | | L (SEQ ID | EKTVAPTE |
| | | (SEQ ID | NO: 587) | | NO: 403) | CS (SEQ |
| | | NO: 309) | | | | ID NO: |
| | | | | | | 416) |
| TA289 | MGWSCII | QVTLKESGP | ASTKGPSVFPLAPSSKSTSGGTAALG | MGWSCII | QAVVTQESA | GQPKAAPS |
| | LFLVATA | GILQPSQTL | CLVKDYFPEPVTVSWNSGALTSGVHT | LFLVATA | LTTSPGETV | VTLFPPSS |
| | TGVHS | SLTCSFSGF | FPAVLQSSGLYSLSSVVTVPSSSLGT | TGVHS | TLTCRSSTG | EELQANKA |
| | (SEQ ID | SLSTFGMGV | QTYICNVNHKPSNTKVDKKVEPKSCD | (SEQ ID | AVTTSNYAN | TLVCLISD |
| | NO: | GWIRQPSGK | KTHTCPPCPAPDLLGDDSVFLFPPKP | NO: | WVQEKPDHL | FYPGAVTV |
| | 588) | GLEWLAHIW | KDTLMISRTPEVTCVVVDVSDEDGEV | 588) | FTGLIGGTN | AWKADSSP |
| | | WDDDKYYNP | KFNWYVDGVEVHNAKTKPREEQYNST | | NRAPGVPAR | VKAGVETT |
| | | ALKSRLTIS | YRVVSVLTVLHQDWLNGKEYKCKVSN | | FSGSLIGDK | TPSKQSNN |
| | | KDTSKNQVF | KALPRPIEKTISKAKGQPREPQVYTL | | AALTITGAQ | KYAASSYL |
| | | LKIANVDTA | PPSRDELTKNQVSLTCLVKGFYPSDI | | TEDEAIYFC | SLTPEQWK |
| | | DTATYYCAR | AVEWESNGQPENNYKTTPPVLDSDGS | | ALWYSNQFI | SHRSYSCQ |
| | | IRGTWWYFD | FFLYSKLTVDKSRWQQGNVFSCSVMH | | FGSGTKVTV | VTHEGSTV |
| | | VWGTGTTVT | EALHNHYTQKSLSLSPG (SEQ ID | | L (SEQ ID | EKTVAPTE |
| | | VSS (SEQ | NO: 587) | | NO: 403) | CS (SEQ |
| | | ID NO: | | | | ID NO: |
| | | 310) | | | | 416) |
| TA290 | MGWSCII | EVQLQQSGP | ASTKGPSVFPLAPSSKSTSGGTAALG | MGWSCII | ENVLTQSQA | RTVAAPSV |
| | LFLVATA | ELVKPGASV | CLVKDYFPEPVTVSWNSGALTSGVHT | LFLVATA | IMSASPGEK | FIFPPSDE |
| | TGVHS | KISCKASGY | FPAVLQSSGLYSLSSVVTVPSSSLGT | TGVHS | VTMTCRASS | QLKSGTAS |
| | (SEQ ID | TFTDYYMNW | QTYICNVNHKPSNTKVDKKVEPKSCD | (SEQ ID | SVSSSYLHW | VVCLLNNF |
| | NO: | VKQSHGKSL | KTHTCPPCPAPDLLGDDSVFLFPPKP | NO: | YQQKSGASP | YPREAKVQ |
| | 588) | EWIGDINPN | KDTLMISRTPEVTCVVVDVSDEDGEV | 588) | KLWIYSTSN | WKVDNALQ |
| | | NGGTSYNQK | KFNWYVDGVEVHNAKTKPREEQYNST | | LASGVPARF | SGNSQESV |
| | | FKGKATLTV | YRVVSVLTVLHQDWLNGKEYKCKVSN | | SGSGSGTSY | TEQDSKDS |
| | | DKSSSTAYM | KALPRPIEKTISKAKGQPREPQVYTL | | SLTISSVEA | TYSLSSTL |
| | | ELRSLTSED | PPSRDELTKNQVSLTCLVKGFYPSDI | | EDAATYYCQ | TLSKADYE |
| | | SAVYYCARR | AVEWESNGQPENNYKTTPPVLDSDGS | | QYSGYPLTF | KHKVYACE |
| | | GGSSSYWYF | FFLYSKLTVDKSRWQQGNVFSCSVMH | | GAGTKLELK | VTHQGLSS |
| | | DVWGTGTTV | EALHNHYTQKSLSLSPG (SEQ ID | | (SEQ ID | PVTKSFNR |
| | | TVSS (SEQ | NO: 587) | | NO: 404) | GEC (SEQ |
| | | ID NO: | | | | ID NO: |
| | | 311) | | | | 415) |
| TA291 | MGWSCII | EVQLQQSGP | ASTKGPSVFPLAPSSKSTSGGTAALG | MGWSCII | DIVLTQSPA | RTVAAPSV |
| | LFLVATA | ELVKPGASV | CLVKDYFPEPVTVSWNSGALTSGVHT | LFLVATA | SLAVSLGQR | FIFPPSDE |
| | TGVHS | KISCKASGY | FPAVLQSSGLYSLSSVVTVPSSSLGT | TGVHS | ATISCRASQ | QLKSGTAS |
| | (SEQ ID | TFTDYYMNW | QTYICNVNHKPSNTKVDKKVEPKSCD | (SEQ ID | SVSTSSYSY | VVCLLNNF |
| | NO: | VKQSHGKSL | KTHTCPPCPAPDLLGDDSVFLFPPKP | NO: | MHWYQQKPG | YPREAKVQ |
| | 588) | EWIGDINPN | KDTLMISRTPEVTCVVVDVSDEDGEV | 588) | QPPKLLIKY | WKVDNALQ |
| | | NGGTSYNQK | KFNWYVDGVEVHNAKTKPREEQYNST | | ASNLESGVP | SGNSQESV |
| | | FKGKATLTV | YRVVSVLTVLHQDWLNGKEYKCKVSN | | ARFSGSGSG | TEQDSKDS |
| | | DKSSSTAYM | KALPRPIEKTISKAKGQPREPQVYTL | | TDFTLNIHP | TYSLSSTL |
| | | ELRSLTSED | PPSRDELTKNQVSLTCLVKGFYPSDI | | VEEEDTATY | TLSKADYE |
| | | SAVYYCARR | AVEWESNGQPENNYKTTPPVLDSDGS | | YCQHSWEIP | KHKVYACE |
| | | GGSSSYWYF | FFLYSKLTVDKSRWQQGNVFSCSVMH | | PTFGSGTKL | VTHQGLSS |
| | | DVWGTGTTV | EALHNHYTQKSLSLSPG (SEQ ID | | EIK (SEQ | PVTKSFNR |
| | | TVSS (SEQ | NO: 587) | | ID NO: | GEC (SEQ |
| | | ID NO: | | | 405) | ID NO: |
| | | 311) | | | | 415) |
| TA292 | MGWSCII | EVQLQQSGP | ASTKGPSVFPLAPSSKSTSGGTAALG | MGWSCII | DIQMTQSPA | RTVAAPSV |
| | LFLVATA | ELVKPGASV | CLVKDYFPEPVTVSWNSGALTSGVHT | LFLVATA | SQSASLGES | FIFPPSDE |
| | TGVHS | KISCKASGY | FPAVLQSSGLYSLSSVVTVPSSSLGT | TGVHS | VTITCLASQ | QLKSGTAS |
| | (SEQ ID | TFTDYYMNW | QTYICNVNHKPSNTKVDKKVEPKSCD | (SEQ ID | TIGTWLAWY | VVCLLNNF |
| | NO: | VKQSHGKSL | KTHTCPPCPAPDLLGDDSVFLFPPKP | NO: | QQKPGKSPQ | YPREAKVQ |
| | 588) | EWIGDINPN | KDTLMISRTPEVTCVVVDVSDEDGEV | 588) | LLIYAATSL | WKVDNALQ |
| | | NGGTSYNQK | KFNWYVDGVEVHNAKTKPREEQYNST | | ADGVPSRES | SGNSQESV |
| | | FKGKATLTV | YRVVSVLTVLHQDWLNGKEYKCKVSN | | GSGSGTKFS | TEQDSKDS |
| | | DKSSSTAYM | KALPRPIEKTISKAKGQPREPQVYTL | | FKISSLQAE | TYSLSSTL |
| | | ELRSLTSED | PPSRDELTKNQVSLTCLVKGFYPSDI | | DFVSYYCQQ | TLSKADYE |
| | | SAVYYCARR | AVEWESNGQPENNYKTTPPVLDSDGS | | FYSTPWTFG | KHKVYACE |
| | | GGSSSYWYF | FFLYSKLTVDKSRWQQGNVFSCSVMH | | GGTKLEIK | VTHQGLSS |
| | | DVWGTGTTV | EALHNHYTQKSLSLSPG (SEQ ID | | (SEQ ID | PVTKSFNR |
| | | TVSS (SEQ | NO: 587) | | NO: 406) | GEC (SEQ |
| | | ID NO: | | | | ID NO: |
| | | 311) | | | | 415) |
| TA293 | MGWSCII | EVQLQQSGP | ASTKGPSVFPLAPSSKSTSGGTAALG | MGWSCII | DIQMTQSPA | RTVAAPSV |
| | LFLVATA | ELVKPGASV | CLVKDYFPEPVTVSWNSGALTSGVHT | LFLVATA | SQSASLGES | FIFPPSDE |
| | TGVHS | KISCKASGY | FPAVLQSSGLYSLSSVVTVPSSSLGT | TGVHS | VTITCLASQ | QLKSGTAS |
| | (SEQ ID | TFTDYYMNW | QTYICNVNHKPSNTKVDKKVEPKSCD | (SEQ ID | TIGTWLAWY | VVCLLNNF |
| | NO: | VKQSHGKSL | KTHTCPPCPAPDLLGDDSVFLFPPKP | NO: | QQKPGKSPQ | YPREAKVQ |
| | 588) | EWIGDINPN | KDTLMISRTPEVTCVVVDVSDEDGEV | 588) | LLIYAATSL | WKVDNALQ |
| | | NGGTSYNQK | KFNWYVDGVEVHNAKTKPREEQYNST | | ADGVPSRFS | SGNSQESV |
| | | FKGKATLTV | YRVVSVLTVLHQDWLNGKEYKCKVSN | | GSGSGTKES | TEQDSKDS |
| | | DKSSSTAYM | KALPRPIEKTISKAKGQPREPQVYTL | | FKISSLQAE | TYSLSSTL |
| | | ELRSLTSED | PPSRDELTKNQVSLTCLVKGFYPSDI | | DFESYYCQQ | TLSKADYE |
| | | SAVYYCARR | AVEWESNGQPENNYKTTPPVLDSDGS | | FYSTPWTFG | KHKVYACE |
| | | GGSSSYWYF | FFLYSKLTVDKSRWQQGNVFSCSVMH | | GGTKLEIK | VTHQGLSS |
| | | DVWGTGTTV | EALHNHYTQKSLSLSPG (SEQ ID | | (SEQ ID | PVTKSFNR |
| | | TVSS (SEQ | NO: 587) | | NO: 407) | GEC (SEQ |
| | | ID NO: | | | | ID NO: |
| | | 311) | | | | 415) |
| TA294 | MGWSCII | QVTLKESGP | ASTKGPSVFPLAPSSKSTSGGTAALG | MGWSCII | DIVLTQSPA | RTVAAPSV |
| | LFLVATA | GILQPSQTL | CLVKDYFPEPVTVSWNSGALTSGVHT | LFLVATA | SLAVSLGQR | FIFPPSDE |
| | TGVHS | SLTCSFSGF | FPAVLQSSGLYSLSSVVTVPSSSLGT | TGVHS | ATISCRASQ | QLKSGTAS |
| | (SEQ ID | SLSTFGMGV | QTYICNVNHKPSNTKVDKKVEPKSCD | (SEQ ID | SVSTSSYSY | VVCLLNNF |
| | NO: | GWIRQPSGK | KTHTCPPCPAPDLLGDDSVFLFPPKP | NO: | MHWYQQKPG | YPREAKVQ |
| | 588) | GLEWLAHIW | KDTLMISRTPEVTCVVVDVSDEDGEV | 588) | QPPKLLIKY | WKVDNALQ |
| | | WDDDKYYNP | KFNWYVDGVEVHNAKTKPREEQYNST | | ASNLESGVP | SGNSQESV |
| | | ALKSRLTIS | YRVVSVLTVLHQDWLNGKEYKCKVSN | | ARFSGSGSG | TEQDSKDS |
| | | KDTSKNQVF | KALPRPIEKTISKAKGQPREPQVYTL | | TDFTLNIHP | TYSLSSTL |
| | | LKIANVDTA | PPSRDELTKNQVSLTCLVKGFYPSDI | | VEEEDTATY | TLSKADYE |
| | | DTATYYCAR | AVEWESNGQPENNYKTTPPVLDSDGS | | YCQHSWEIP | KHKVYACE |
| | | KVGRPLWYF | FFLYSKLTVDKSRWQQGNVFSCSVMH | | YTFGGGTKL | VTHQGLSS |
| | | DVWGAGTTV | EALHNHYTQKSLSLSPG (SEQ ID | | EIK (SEQ | PVTKSFNR |
| | | TVSS (SEQ | NO: 587) | | ID NO: | GEC (SEQ |
| | | ID NO: | | | 408) | ID NO: |
| | | 312) | | | | 415) |
| TA295 | MGWSCII | QVTLKESGP | ASTKGPSVFPLAPSSKSTSGGTAALG | MGWSCII | QAVVTQESA | GQPKAAPS |
| | LFLVATA | GILQPSQTL | CLVKDYFPEPVTVSWNSGALTSGVHT | LFLVATA | LTTSPGGTV | VTLFPPSS |
| | TGVHS | SLTCSFSGF | FPAVLQSSGLYSLSSVVTVPSSSLGT | TGVHS | ILTCRSSTG | EELQANKA |
| | (SEQ ID | SLSTFGMGV | QTYICNVNHKPSNTKVDKKVEPKSCD | (SEQ ID | AVTTSYYAN | TLVCLISD |
| | NO: | GWIRQPSGK | KTHTCPPCPAPDLLGDDSVFLFPPKP | NO: | WVQEKPDHL | FYPGAVTV |
| | 588) | GLEWLAHIW | KDTLMISRTPEVTCVVVDVSDEDGEV | 588) | FTGLIGGTS | AWKADSSP |
| | | WDDDKYYNP | KFNWYVDGVEVHNAKTKPREEQYNST | | NRAPGVPVR | VKAGVETT |
| | | ALKSRLTIS | YRVVSVLTVLHQDWLNGKEYKCKVSN | | FSGSLIGDK | TPSKQSNN |
| | | KDTSKNQVF | KALPRPIEKTISKAKGQPREPQVYTL | | AALTITGAQ | KYAASSYL |
| | | LKIANVDTA | PPSRDELTKNQVSLTCLVKGFYPSDI | | TEDDAMYFC | SLTPEQWK |
| | | DTATYYCAR | AVEWESNGQPENNYKTTPPVLDSDGS | | ALWYSTHYV | SHRSYSCQ |
| | | KVGRPLWYF | FFLYSKLTVDKSRWQQGNVFSCSVMH | | FGGGTKVTV | VTHEGSTV |
| | | DVWGAGTTV | EALHNHYTQKSLSLSPG (SEQ ID | | L (SEQ ID | EKTVAPTE |
| | | TVSS (SEQ | NO: 587) | | NO: 409) | CS (SEQ |
| | | ID NO: | | | | ID NO: |
| | | 312) | | | | 416) |
| TA296 | MGWSCII | QVQLKESGP | ASTKGPSVFPLAPSSKSTSGGTAALG | MGWSCII | DIVLTQSPA | RTVAAPSV |
| | LFLVATA | GLVAPSQSL | CLVKDYFPEPVTVSWNSGALTSGVHT | LFLVATA | SLAVSLGQR | FIFPPSDE |
| | TGVHS | SITCTVSGF | FPAVLQSSGLYSLSSVVTVPSSSLGT | TGVHS | ATISCRASQ | QLKSGTAS |
| | (SEQ ID | SLTSYAISW | QTYICNVNHKPSNTKVDKKVEPKSCD | (SEQ ID | SVSTSSYSY | VVCLLNNF |
| | NO: | VRQPPGKGL | KTHTCPPCPAPDLLGDDSVFLFPPKP | NO: | MHWYQQKPG | YPREAKVQ |
| | 588) | EWLGVIWTG | KDTLMISRTPEVTCVVVDVSDEDGEV | 588) | QPPKLLIKY | WKVDNALQ |
| | | GGTNYNSAL | KFNWYVDGVEVHNAKTKPREEQYNST | | ASNLESGVP | SGNSQESV |
| | | KSRLSISKD | YRVVSVLTVLHQDWLNGKEYKCKVSN | | ARFSGSGSG | TEQDSKDS |
| | | NSKSQVELK | KALPRPIEKTISKAKGQPREPQVYTL | | TDFTLNIHP | TYSLSSTL |
| | | MNSLQTDDT | PPSRDELTKNQVSLTCLVKGFYPSDI | | VEEEDTATY | TLSKADYE |
| | | ARYYCASSK | AVEWESNGQPENNYKTTPPVLDSDGS | | YCQHSWEIP | KHKVYACE |
| | | GSYYAMDYW | FFLYSKLTVDKSRWQQGNVFSCSVMH | | YTFGGGTKL | VTHQGLSS |
| | | GQGTSVTVS | EALHNHYTQKSLSLSPG (SEQ ID | | EIK (SEQ | PVTKSFNR |
| | | S (SEQ ID | NO: 587) | | ID NO: | GEC (SEQ |
| | | NO: 313) | | | 408) | ID NO: |
| | | | | | | 415) |
| TA297 | MGWSCII | QVQLKESGP | ASTKGPSVFPLAPSSKSTSGGTAALG | MGWSCII | QAVVTQESA | GQPKAAPS |
| | LFLVATA | GLVAPSQSL | CLVKDYFPEPVTVSWNSGALTSGVHT | LFLVATA | LTTSPGGTV | VTLFPPSS |
| | TGVHS | SITCTVSGF | FPAVLQSSGLYSLSSVVTVPSSSLGT | TGVHS | ILTCRSSTG | EELQANKA |
| | (SEQ ID | SLTSYAISW | QTYICNVNHKPSNTKVDKKVEPKSCD | (SEQ ID | AVTTSYYAN | TLVCLISD |
| | NO: | VRQPPGKGL | KTHTCPPCPAPDLLGDDSVFLFPPKP | NO: | WVQEKPDHL | FYPGAVTV |
| | 588) | EWLGVIWTG | KDTLMISRTPEVTCVVVDVSDEDGEV | 588) | FTGLIGGTS | AWKADSSP |
| | | GGTNYNSAL | KFNWYVDGVEVHNAKTKPREEQYNST | | NRAPGVPVR | VKAGVETT |
| | | KSRLSISKD | YRVVSVLTVLHQDWLNGKEYKCKVSN | | FSGSLIGDK | TPSKQSNN |
| | | NSKSQVELK | KALPRPIEKTISKAKGQPREPQVYTL | | AALTITGAQ | KYAASSYL |
| | | MNSLQTDDT | PPSRDELTKNQVSLTCLVKGFYPSDI | | TEDDAMYFC | SLTPEQWK |
| | | ARYYCASSK | AVEWESNGQPENNYKTTPPVLDSDGS | | ALWYSTHYV | SHRSYSCQ |
| | | GSYYAMDYW | FFLYSKLTVDKSRWQQGNVFSCSVMH | | FGGGTKVTV | VTHEGSTV |
| | | GQGTSVTVS | EALHNHYTQKSLSLSPG (SEQ ID | | L (SEQ ID | EKTVAPTE |
| | | S (SEQ ID | NO: 587) | | NO: 409) | CS (SEQ |
| | | NO: 313) | | | | ID NO: |
| | | | | | | 416) |
| TA298 | MGWSCII | QIQLVQSGP | ASTKGPSVFPLAPSSKSTSGGTAALG | MGWSCII | QIVLTQSPT | RTVAAPSV |
| | LFLVATA | ELKKPGATV | CLVKDYFPEPVTVSWNSGALTSGVHT | LFLVATA | IMSASPGEK | FIFPPSDE |
| | TGVHS | KISCKASGF | FPAVLQSSGLYSLSSVVTVPSSSLGT | TGVHS | VTMTCSASL | QLKSGTAS |
| | (SEQ ID | TFTTYGMSW | QTYICNVNHKPSNTKVDKKVEPKSCD | (SEQ ID | SLSSMFWYQ | VVCLLNNF |
| | NO: | VKQAPGKGF | KTHTCPPCPAPDLLGDDSVFLFPPKP | NO: | QKPGSSPRL | YPREAKVQ |
| | 588) | KWMGWLNTY | KDTLMISRTPEVTCVVVDVSDEDGEV | 588) | LIYDTSKLA | WKVDNALQ |
| | | SGVPTYADD | KFNWYVDGVEVHNAKTKPREEQYNST | | SGVPVRFSG | SGNSQESV |
| | | FKGRFAFSL | YRVVSVLTVLHQDWLNGKEYKCKVSN | | SGSGTSYSL | TEQDSKDS |
| | | ETSASTAYL | KALPRPIEKTISKAKGQPREPQVYTL | | TISRMEAED | TYSLSSTL |
| | | QINNLKNED | PPSRDELTKNQVSLTCLVKGFYPSDI | | GATYYCQQW | TLSKADYE |
| | | TATYFCARG | AVEWESNGQPENNYKTTPPVLDSDGS | | SSFPFTEGS | KHKVYACE |
| | | GYPYWGRGT | FFLYSKLTVDKSRWQQGNVFSCSVMH | | GTKLEIK | VTHQGLSS |
| | | TLTVSS | EALHNHYTQKSLSLSPG (SEQ ID | | (SEQ ID | PVTKSFNR |
| | | (SEQ ID | NO: 587) | | NO: 410) | GEC (SEQ |
| | | NO: 314) | | | | ID NO: |
| | | | | | | 415) |
| TA299 | MGWSCII | QIQLVQSGP | ASTKGPSVFPLAPSSKSTSGGTAALG | MGWSCII | DIKMTQSPS | RTVAAPSV |
| | LFLVATA | ELKKPGATV | CLVKDYFPEPVTVSWNSGALTSGVHT | LFLVATA | SMYASLGER | FIFPPSDE |
| | TGVHS | KISCKASGF | FPAVLQSSGLYSLSSVVTVPSSSLGT | TGVHS | VTITCKASQ | QLKSGTAS |
| | (SEQ ID | TFTTYGMSW | QTYICNVNHKPSNTKVDKKVEPKSCD | (SEQ ID | DINSYLSWF | VVCLLNNF |
| | NO: | VKQAPGKGF | KTHTCPPCPAPDLLGDDSVFLFPPKP | NO: | QQKPGKSPK | YPREAKVQ |
| | 588) | KWMGWLNTY | KDTLMISRTPEVTCVVVDVSDEDGEV | 588) | TLIYRANRL | WKVDNALQ |
| | | SGVPTYADD | KFNWYVDGVEVHNAKTKPREEQYNST | | VDGVPSRFS | SGNSQESV |
| | | FKGRFAFSL | YRVVSVLTVLHQDWLNGKEYKCKVSN | | GSGSGQDYS | TEQDSKDS |
| | | ETSASTAYL | KALPRPIEKTISKAKGQPREPQVYTL | | LTISSLEYE | TYSLSSTL |
| | | QINNLKNED | PPSRDELTKNQVSLTCLVKGFYPSDI | | DMGIYYCLQ | TLSKADYE |
| | | TATYFCARG | AVEWESNGQPENNYKTTPPVLDSDGS | | YDEFPYTFG | KHKVYACE |
| | | GYPYWGRGT | FFLYSKLTVDKSRWQQGNVFSCSVMH | | GGTKLEIK | VTHQGLSS |
| | | TLTVSS | EALHNHYTQKSLSLSPG (SEQ ID | | (SEQ ID | PVTKSFNR |
| | | (SEQ ID | NO: 587) | | NO: 411) | GEC (SEQ |
| | | NO: 314) | | | | ID NO: |
| | | | | | | 415) |
| TA300 | MGWSCII | EVQLQQSGA | ASTKGPSVFPLAPSSKSTSGGTAALG | MGWSCII | QIVLTQSPT | RTVAAPSV |
| | LFLVATA | ELVKPGASV | CLVKDYFPEPVTVSWNSGALTSGVHT | LFLVATA | IMSASPGEK | FIFPPSDE |
| | TGVHS | KLSCTASGF | FPAVLQSSGLYSLSSVVTVPSSSLGT | TGVHS | VTMTCSASL | QLKSGTAS |
| | (SEQ ID | NIKDYYMHW | QTYICNVNHKPSNTKVDKKVEPKSCD | (SEQ ID | SLSSMFWYQ | VVCLLNNF |
| | NO: | VKQRTEQGL | KTHTCPPCPAPDLLGDDSVFLFPPKP | NO: | QKPGSSPRL | YPREAKVQ |
| | 588) | EWIGRIDPE | KDTLMISRTPEVTCVVVDVSDEDGEV | 588) | LIYDTSKLA | WKVDNALQ |
| | | DGETKYAPK | KFNWYVDGVEVHNAKTKPREEQYNST | | SGVPVRFSG | SGNSQESV |
| | | FQGKATITA | YRVVSVLTVLHQDWLNGKEYKCKVSN | | SGSGTSYSL | TEQDSKDS |
| | | DTSSNTAYL | KALPRPIEKTISKAKGQPREPQVYTL | | TISRMEAED | TYSLSSTL |
| | | QLSSLTSED | PPSRDELTKNQVSLTCLVKGFYPSDI | | GATYYCQQW | TLSKADYE |
| | | TAVYYCGRD | AVEWESNGQPENNYKTTPPVLDSDGS | | SSFPFTEGS | KHKVYACE |
| | | GYYDVYFDY | FFLYSKLTVDKSRWQQGNVFSCSVMH | | GTKLEIK | VTHQGLSS |
| | | WGQGTTLTV | EALHNHYTQKSLSLSPG (SEQ ID | | (SEQ ID | PVTKSFNR |
| | | SS (SEQ | NO: 587) | | NO: 410) | GEC (SEQ |
| | | ID NO: | | | | ID NO: |
| | | 315) | | | | 415) |
| TA301 | MGWSCII | EVQLQQSGA | ASTKGPSVFPLAPSSKSTSGGTAALG | MGWSCII | DIKMTQSPS | RTVAAPSV |
| | LFLVATA | ELVKPGASV | CLVKDYFPEPVTVSWNSGALTSGVHT | LFLVATA | SMYASLGER | FIFPPSDE |
| | TGVHS | KLSCTASGF | FPAVLQSSGLYSLSSVVTVPSSSLGT | TGVHS | VTITCKASQ | QLKSGTAS |
| | (SEQ ID | NIKDYYMHW | QTYICNVNHKPSNTKVDKKVEPKSCD | (SEQ ID | DINSYLSWF | VVCLLNNF |
| | NO: | VKQRTEQGL | KTHTCPPCPAPDLLGDDSVFLFPPKP | NO: | QQKPGKSPK | YPREAKVQ |
| | 588) | EWIGRIDPE | KDTLMISRTPEVTCVVVDVSDEDGEV | 588) | TLIYRANRL | WKVDNALQ |
| | | DGETKYAPK | KFNWYVDGVEVHNAKTKPREEQYNST | | VDGVPSRES | SGNSQESV |
| | | FQGKATITA | YRVVSVLTVLHQDWLNGKEYKCKVSN | | GSGSGQDYS | TEQDSKDS |
| | | DTSSNTAYL | KALPRPIEKTISKAKGQPREPQVYTL | | LTISSLEYE | TYSLSSTL |
| | | QLSSLTSED | PPSRDELTKNQVSLTCLVKGFYPSDI | | DMGIYYCLQ | TLSKADYE |
| | | TAVYYCGRD | AVEWESNGQPENNYKTTPPVLDSDGS | | YDEFPYTFG | KHKVYACE |
| | | GYYDVYFDY | FFLYSKLTVDKSRWQQGNVFSCSVMH | | GGTKLEIK | VTHQGLSS |
| | | WGQGTTLTV | EALHNHYTQKSLSLSPG (SEQ ID | | (SEQ ID | PVTKSFNR |
| | | SS (SEQ | NO: 587) | | NO: 411) | GEC (SEQ |
| | | ID NO: | | | | ID NO: |
| | | 315) | | | | 415) |
| TA302 | MGWSCII | QVQLQQSGA | ASTKGPSVFPLAPSSKSTSGGTAALG | MGWSCII | QIVLTQSPT | RTVAAPSV |
| | LFLVATA | ELVKPGASV | CLVKDYFPEPVTVSWNSGALTSGVHT | LFLVATA | IMSASPGEK | FIFPPSDE |
| | TGVHS | KISCKTSGY | FPAVLQSSGLYSLSSVVTVPSSSLGT | TGVHS | VTMTCSASL | QLKSGTAS |
| | (SEQ ID | IFSNYWMNW | QTYICNVNHKPSNTKVDKKVEPKSCD | (SEQ ID | SLSSMFWYQ | VVCLLNNF |
| | NO: | VKQRPGKGL | KTHTCPPCPAPDLLGDDSVFLFPPKP | NO: | QKPGSSPRL | YPREAKVQ |
| | 588) | EWIGQIYPG | KDTLMISRTPEVTCVVVDVSDEDGEV | 588) | LIYDTSKLA | WKVDNALQ |
| | | DGDTNYNGD | KFNWYVDGVEVHNAKTKPREEQYNST | | SGVPVRFSG | SGNSQESV |
| | | FKGKATLTA | YRVVSVLTVLHQDWLNGKEYKCKVSN | | SGSGTSYSL | TEQDSKDS |
| | | DKSSSTAYI | KALPRPIEKTISKAKGQPREPQVYTL | | TISRMEAED | TYSLSSTL |
| | | YLNSLTSED | PPSRDELTKNQVSLTCLVKGFYPSDI | | GATYYCQQW | TLSKADYE |
| | | SAVYFCARG | AVEWESNGQPENNYKTTPPVLDSDGS | | SSFPFTEGS | KHKVYACE |
| | | PYWGLGTLV | FFLYSKLTVDKSRWQQGNVFSCSVMH | | GTKLEIK | VTHQGLSS |
| | | TVSA (SEQ | EALHNHYTQKSLSLSPG (SEQ ID | | (SEQ ID | PVTKSFNR |
| | | ID NO: | NO: 587) | | NO: 410) | GEC (SEQ |
| | | 316) | | | | ID NO: |
| | | | | | | 415) |
| TA303 | MGWSCII | QVQLQQSGA | ASTKGPSVFPLAPSSKSTSGGTAALG | MGWSCII | DIKMTQSPS | RTVAAPSV |
| | LFLVATA | ELVKPGASV | CLVKDYFPEPVTVSWNSGALTSGVHT | LFLVATA | SMYASLGER | FIFPPSDE |
| | TGVHS | KISCKTSGY | FPAVLQSSGLYSLSSVVTVPSSSLGT | TGVHS | VTITCKASQ | QLKSGTAS |
| | (SEQ ID | IFSNYWMNW | QTYICNVNHKPSNTKVDKKVEPKSCD | (SEQ ID | DINSYLSWF | VVCLLNNF |
| | NO: | VKQRPGKGL | KTHTCPPCPAPDLLGDDSVFLFPPKP | NO: | QQKPGKSPK | YPREAKVQ |
| | 588) | EWIGQIYPG | KDTLMISRTPEVTCVVVDVSDEDGEV | 588) | TLIYRANRL | WKVDNALQ |
| | | DGDTNYNGD | KFNWYVDGVEVHNAKTKPREEQYNST | | VDGVPSRES | SGNSQESV |
| | | FKGKATLTA | YRVVSVLTVLHQDWLNGKEYKCKVSN | | GSGSGQDYS | TEQDSKDS |
| | | DKSSSTAYI | KALPRPIEKTISKAKGQPREPQVYTL | | LTISSLEYE | TYSLSSTL |
| | | YLNSLTSED | PPSRDELTKNQVSLTCLVKGFYPSDI | | DMGIYYCLQ | TLSKADYE |
| | | SAVYFCARG | AVEWESNGQPENNYKTTPPVLDSDGS | | YDEFPYTFG | KHKVYACE |
| | | PYWGLGTLV | FFLYSKLTVDKSRWQQGNVFSCSVMH | | GGTKLEIK | VTHQGLSS |
| | | TVSA (SEQ | EALHNHYTQKSLSLSPG (SEQ ID | | (SEQ ID | PVTKSFNR |
| | | ID NO: | NO: 587) | | NO: 411) | GEC (SEQ |
| | | 316) | | | | ID NO: |
| | | | | | | 415) |
| TA304 | MGWSCII | QVTLKESGP | ASTKGPSVFPLAPSSKSTSGGTAALG | MGWSCII | QAVVTQESA | GQPKAAPS |
| | LFLVATA | GILQPSQTL | CLVKDYFPEPVTVSWNSGALTSGVHT | LFLVATA | LTTSPGETV | VTLFPPSS |
| | TGVHS | SLTCSFSGF | FPAVLQSSGLYSLSSVVTVPSSSLGT | TGVHS | TLTCRSSTG | EELQANKA |
| | (SEQ ID | SLSTFGMGV | QTYICNVNHKPSNTKVDKKVEPKSCD | (SEQ ID | AVTTSNYAN | TLVCLISD |
| | NO: | GWIRQPSGK | KTHTCPPCPAPDLLGDDSVFLFPPKP | NO: | WVQEKPDHL | FYPGAVTV |
| | 588) | GLEWLAHIW | KDTLMISRTPEVTCVVVDVSDEDGEV | 588) | FTGLIGGTN | AWKADSSP |
| | | WDDDKYYNP | KFNWYVDGVEVHNAKTKPREEQYNST | | NRAPGVPAR | VKAGVETT |
| | | ALKSRLTIS | YRVVSVLTVLHQDWLNGKEYKCKVSN | | FSGSLIGDK | TPSKQSNN |
| | | KDTSKNQVF | KALPRPIEKTISKAKGQPREPQVYTL | | AALTITGAQ | KYAASSYL |
| | | LKIANVDTA | PPSRDELTKNQVSLTCLVKGFYPSDI | | TEDEAIYFC | SLTPEQWK |
| | | DTATYYCAR | AVEWESNGQPENNYKTTPPVLDSDGS | | ALWYSNHYI | SHRSYSCQ |
| | | IQSFYWYFD | FFLYSKLTVDKSRWQQGNVFSCSVMH | | FGSGTKVTV | VTHEGSTV |
| | | VWGTGTTVT | EALHNHYTQKSLSLSPG (SEQ ID | | L (SEQ ID | EKTVAPTE |
| | | VSS (SEQ | NO: 587) | | NO: 412) | CS (SEQ |
| | | ID NO: | | | | ID NO: |
| | | 317) | | | | 416) |
| TA305 | MGWSCII | EVQLQQSGA | ASTKGPSVFPLAPSSKSTSGGTAALG | MGWSCII | QAVVTQESA | GQPKAAPS |
| | LFLVATA | ELVKPGASV | CLVKDYFPEPVTVSWNSGALTSGVHT | LFLVATA | LTTSPGETV | VTLFPPSS |
| | TGVHS | KLSCTASGF | FPAVLQSSGLYSLSSVVTVPSSSLGT | TGVHS | TLTCRSSTG | EELQANKA |
| | (SEQ ID | NIKDYYMHW | QTYICNVNHKPSNTKVDKKVEPKSCD | (SEQ ID | AVTTSNYAN | TLVCLISD |
| | NO: | VKQRTEQGL | KTHTCPPCPAPDLLGDDSVFLFPPKP | NO: | WVQEKPDHL | FYPGAVTV |
| | 588) | EWIGRIDPE | KDTLMISRTPEVTCVVVDVSDEDGEV | 588) | FTGLIGGTN | AWKADSSP |
| | | DGETKYAPK | KFNWYVDGVEVHNAKTKPREEQYNST | | NRAPGVPAR | VKAGVETT |
| | | FQGKTTITA | YRVVSVLTVLHQDWLNGKEYKCKVSN | | FSGSLIGDK | TPSKQSNN |
| | | DTSSNTAYL | KALPRPIEKTISKAKGQPREPQVYTL | | AALTITGAQ | KYAASSYL |
| | | QLSSLTSED | PPSRDELTKNQVSLTCLVKGFYPSDI | | TEDEAIYFC | SLTPEQWK |
| | | TAVYYCGRD | AVEWESNGQPENNYKTTPPVLDSDGS | | ALWYSNHYI | SHRSYSCQ |
| | | GYYDVYFDY | FFLYSKLTVDKSRWQQGNVFSCSVMH | | FGSGTKVTV | VTHEGSTV |
| | | WGQGTTLTV | EALHNHYTQKSLSLSPG (SEQ ID | | L (SEQ ID | EKTVAPTE |
| | | SS (SEQ | NO: 587) | | NO: 412) | CS (SEQ |
| | | ID NO: | | | | ID NO: |
| | | 318) | | | | 416) |
| TA306 | MGWSCII | QIQLVQSGP | ASTKGPSVFPLAPSSKSTSGGTAALG | MGWSCII | DVVMTQTPL | RTVAAPSV |
| | LFLVATA | ELKKPGETV | CLVKDYFPEPVTVSWNSGALTSGVHT | LFLVATA | TLSVTIGQP | FIFPPSDE |
| | TGVHS | KISCKASGY | FPAVLQSSGLYSLSSVVTVPSSSLGT | TGVHS | ASISCKSSQ | QLKSGTAS |
| | (SEQ ID | TFTTYGMSW | QTYICNVNHKPSNTKVDKKVEPKSCD | (SEQ ID | SLLDSDGKT | VVCLLNNF |
| | NO: | VKQAPGKGL | KTHTCPPCPAPDLLGDDSVFLFPPKP | NO: | YLNWLLQRP | YPREAKVQ |
| | 588) | KWMGWINTY | KDTLMISRTPEVTCVVVDVSDEDGEV | 588) | GQSPKRLIY | WKVDNALQ |
| | | SGLPTYADD | KFNWYVDGVEVHNAKTKPREEQYNST | | LVSKLDSGV | SGNSQESV |
| | | FKGRFAFSL | YRVVSVLTVLHQDWLNGKEYKCKVSN | | PDRFTGSGS | TEQDSKDS |
| | | ETSASTAYL | KALPRPIEKTISKAKGQPREPQVYTL | | GTDFTLKIS | TYSLSSTL |
| | | QINNLKNED | PPSRDELTKNQVSLTCLVKGFYPSDI | | RVEAEDLGV | TLSKADYE |
| | | TATYFCARG | AVEWESNGQPENNYKTTPPVLDSDGS | | YYCWQGTHE | KHKVYACE |
| | | GYDYGAAYW | FFLYSKLTVDKSRWQQGNVFSCSVMH | | PHTFGAGTK | VTHQGLSS |
| | | GQGTLVTVS | EALHNHYTQKSLSLSPG (SEQ ID | | LELK (SEQ | PVTKSFNR |
| | | A (SEQ ID | NO: 587) | | ID NO: | GEC (SEQ |
| | | NO: 319) | | | 413) | ID NO: |
| | | | | | | 415) |
| TA307 | MGWSCII | QIQLVQSGP | ASTKGPSVFPLAPSSKSTSGGTAALG | MGWSCII | QAVVTQESA | GQPKAAPS |
| | LFLVATA | ELKKPGETV | CLVKDYFPEPVTVSWNSGALTSGVHT | LFLVATA | LTTSPGETV | VTLFPPSS |
| | TGVHS | KISCKASGY | FPAVLQSSGLYSLSSVVTVPSSSLGT | TGVHS | TLTCRSSTG | EELQANKA |
| | (SEQ ID | TFTTYGMSW | QTYICNVNHKPSNTKVDKKVEPKSCD | (SEQ ID | AVTTSNYAN | TLVCLISD |
| | NO: | VKQAPGKGL | KTHTCPPCPAPDLLGDDSVFLFPPKP | NO: | WVQEKPDHL | FYPGAVTV |
| | 588) | KWMGWINTY | KDTLMISRTPEVTCVVVDVSDEDGEV | 588) | FTGLIGGTN | AWKADSSP |
| | | SGLPTYADD | KFNWYVDGVEVHNAKTKPREEQYNST | | NRAPGVPAR | VKAGVETT |
| | | FKGRFAFSL | YRVVSVLTVLHQDWLNGKEYKCKVSN | | FSGSLIGDK | TPSKQSNN |
| | | ETSASTAYL | KALPRPIEKTISKAKGQPREPQVYTL | | AALTITGAQ | KYAASSYL |
| | | QINNLKNED | PPSRDELTKNQVSLTCLVKGFYPSDI | | TEDEAIYFC | SLTPEQWK |
| | | TATYFCARG | AVEWESNGQPENNYKTTPPVLDSDGS | | ALWYSNHLV | SHRSYSCQ |
| | | GYDYGAAYW | FFLYSKLTVDKSRWQQGNVFSCSVMH | | FGGGTKLTV | VTHEGSTV |
| | | GQGTLVTVS | EALHNHYTQKSLSLSPG (SEQ ID | | L (SEQ ID | EKTVAPTE |
| | | A (SEQ ID | NO: 587) | | NO: 391) | CS (SEQ |
| | | NO: 319) | | | | ID NO: |
| | | | | | | 416) |
| TA308 | MGWSCII | QVTLKESGP | ASTKGPSVFPLAPSSKSTSGGTAALG | MGWSCII | DVVMTQTPL | RTVAAPSV |
| | LFLVATA | GILQPSQTL | CLVKDYFPEPVTVSWNSGALTSGVHT | LFLVATA | TLSVTIGQP | FIFPPSDE |
| | TGVHS | SLTCSFSGF | FPAVLQSSGLYSLSSVVTVPSSSLGT | TGVHS | ASISCKSSQ | QLKSGTAS |
| | (SEQ ID | SLSTFGMGV | QTYICNVNHKPSNTKVDKKVEPKSCD | (SEQ ID | SLLDSDGKT | VVCLLNNF |
| | NO: | GWIRQPSGK | KTHTCPPCPAPDLLGDDSVFLFPPKP | NO: | YLNWLLQRP | YPREAKVQ |
| | 588) | GLEWLTHIW | KDTLMISRTPEVTCVVVDVSDEDGEV | 588) | GQSPKRLIY | WKVDNALQ |
| | | WDDDKYYNP | KFNWYVDGVEVHNAKTKPREEQYNST | | LVSKLDSGV | SGNSQESV |
| | | ALKSRLTIS | YRVVSVLTVLHQDWLNGKEYKCKVSN | | PDRFTGSGS | TEQDSKDS |
| | | KDTSKNQVF | KALPRPIEKTISKAKGQPREPQVYTL | | GTDFTLKIS | TYSLSSTL |
| | | LKIANVDTA | PPSRDELTKNQVSLTCLVKGFYPSDI | | RVEAEDLGV | TLSKADYE |
| | | DTATYYCAR | AVEWESNGQPENNYKTTPPVLDSDGS | | YYCWQGTHE | KHKVYACE |
| | | VRMSHWYFD | FFLYSKLTVDKSRWQQGNVFSCSVMH | | PHTFGAGTK | VTHQGLSS |
| | | VWATGTTVT | EALHNHYTQKSLSLSPG (SEQ ID | | LELK (SEQ | PVTKSFNR |
| | | VSS (SEQ | NO: 587) | | ID NO: | GEC (SEQ |
| | | ID NO: | | | 413) | ID NO: |
| | | 320) | | | | 415) |
| TA309 | MGWSCII | QVTLKESGP | ASTKGPSVFPLAPSSKSTSGGTAALG | MGWSCII | QAVVTQESA | GQPKAAPS |
| | LFLVATA | GILQPSQTL | CLVKDYFPEPVTVSWNSGALTSGVHT | LFLVATA | LTTSPGETV | VTLFPPSS |
| | TGVHS | SLTCSFSGF | FPAVLQSSGLYSLSSVVTVPSSSLGT | TGVHS | TLTCRSSTG | EELQANKA |
| | (SEQ ID | SLSTFGMGV | QTYICNVNHKPSNTKVDKKVEPKSCD | (SEQ ID | AVTTSNYAN | TLVCLISD |
| | NO: | GWIRQPSGK | KTHTCPPCPAPDLLGDDSVFLFPPKP | NO: | WVQEKPDHL | FYPGAVTV |
| | 588) | GLEWLTHIW | KDTLMISRTPEVTCVVVDVSDEDGEV | 588) | FTGLIGGTN | AWKADSSP |
| | | WDDDKYYNP | KFNWYVDGVEVHNAKTKPREEQYNST | | NRAPGVPAR | VKAGVETT |
| | | ALKSRLTIS | YRVVSVLTVLHQDWLNGKEYKCKVSN | | FSGSLIGDK | TPSKQSNN |
| | | KDTSKNQVF | KALPRPIEKTISKAKGQPREPQVYTL | | AALTITGAQ | KYAASSYL |
| | | LKIANVDTA | PPSRDELTKNQVSLTCLVKGFYPSDI | | TEDEAIYFC | SLTPEQWK |
| | | DTATYYCAR | AVEWESNGQPENNYKTTPPVLDSDGS | | ALWYSNHLV | SHRSYSCQ |
| | | VRMSHWYFD | FFLYSKLTVDKSRWQQGNVFSCSVMH | | FGGGTKLTV | VTHEGSTV |
| | | VWATGTTVT | EALHNHYTQKSLSLSPG (SEQ ID | | L (SEQ ID | EKTVAPTE |
| | | VSS (SEQ | NO: 587) | | NO: 391) | CS (SEQ |
| | | ID NO: | | | | ID NO: |
| | | 320) | | | | 416) |
| TA310 | MGWSCII | QVQLQQSGA | ASTKGPSVFPLAPSSKSTSGGTAALG | MGWSCII | DVVMTQTPL | RTVAAPSV |
| | LFLVATA | ELMKPGASV | CLVKDYFPEPVTVSWNSGALTSGVHT | LFLVATA | TLSVTIGQP | FIFPPSDE |
| | TGVHS | KLSCKATGY | FPAVLQSSGLYSLSSVVTVPSSSLGT | TGVHS | ASISCKSSQ | QLKSGTAS |
| | (SEQ ID | TFTGYWIEW | QTYICNVNHKPSNTKVDKKVEPKSCD | (SEQ ID | SLLDSDGKT | VVCLLNNF |
| | NO: | VKQRPGHGL | KTHTCPPCPAPDLLGDDSVFLFPPKP | NO: | YLNWLLQRP | YPREAKVQ |
| | 588) | EWIGEILPG | KDTLMISRTPEVTCVVVDVSDEDGEV | 588) | GQSPKRLIY | WKVDNALQ |
| | | SGSTNYNEK | KFNWYVDGVEVHNAKTKPREEQYNST | | LVSKLDSGV | SGNSQESV |
| | | FKGKATFTA | YRVVSVLTVLHQDWLNGKEYKCKVSN | | PDRFTGSGS | TEQDSKDS |
| | | DTSSNTAYM | KALPRPIEKTISKAKGQPREPQVYTL | | GTDFTLKIS | TYSLSSTL |
| | | QLSSLTTED | PPSRDELTKNQVSLTCLVKGFYPSDI | | RVEAEDLGV | TLSKADYE |
| | | SAIHYCARK | AVEWESNGQPENNYKTTPPVLDSDGS | | YYCWQGTHE | KHKVYACE |
| | | EDGYYLYYF | FFLYSKLTVDKSRWQQGNVFSCSVMH | | PHTFGAGTK | VTHQGLSS |
| | | DYWGQGTTL | EALHNHYTQKSLSLSPG (SEQ ID | | LELK (SEQ | PVTKSFNR |
| | | TVSS (SEQ | NO: 587) | | ID NO: | GEC (SEQ |
| | | ID NO: | | | 413) | ID NO: |
| | | 321) | | | | 415) |
| TA311 | MGWSCII | QVQLQQSGA | ASTKGPSVFPLAPSSKSTSGGTAALG | MGWSCII | QAVVTQESA | GQPKAAPS |
| | LFLVATA | ELMKPGASV | CLVKDYFPEPVTVSWNSGALTSGVHT | LFLVATA | LTTSPGETV | VTLFPPSS |
| | TGVHS | KLSCKATGY | FPAVLQSSGLYSLSSVVTVPSSSLGT | TGVHS | TLTCRSSTG | EELQANKA |
| | (SEQ ID | TFTGYWIEW | QTYICNVNHKPSNTKVDKKVEPKSCD | (SEQ ID | AVTTSNYAN | TLVCLISD |
| | NO: | VKQRPGHGL | KTHTCPPCPAPDLLGDDSVFLFPPKP | NO: | WVQEKPDHL | FYPGAVTV |
| | 588) | EWIGEILPG | KDTLMISRTPEVTCVVVDVSDEDGEV | 588) | FTGLIGGTN | AWKADSSP |
| | | SGSTNYNEK | KFNWYVDGVEVHNAKTKPREEQYNST | | NRAPGVPAR | VKAGVETT |
| | | FKGKATFTA | YRVVSVLTVLHQDWLNGKEYKCKVSN | | FSGSLIGDK | TPSKQSNN |
| | | DTSSNTAYM | KALPRPIEKTISKAKGQPREPQVYTL | | AALTITGAQ | KYAASSYL |
| | | QLSSLITED | PPSRDELTKNQVSLTCLVKGFYPSDI | | TEDEAIYFC | SLTPEQWK |
| | | SAIHYCARK | AVEWESNGQPENNYKTTPPVLDSDGS | | ALWYSNHLV | SHRSYSCQ |
| | | EDGYYLYYF | FFLYSKLTVDKSRWQQGNVFSCSVMH | | FGGGTKLTV | VTHEGSTV |
| | | DYWGQGTTL | EALHNHYTQKSLSLSPG (SEQ ID | | L (SEQ ID | EKTVAPTE |
| | | TVSS (SEQ | NO: 587) | | NO: 391) | CS (SEQ |
| | | ID NO: | | | | ID NO: |
| | | 321) | | | | 416) |
| TA312 | MGWSCII | QVQLQQSGA | ASTKGPSVFPLAPSSKSTSGGTAALG | MGWSCII | DIQMTQTTS | RTVAAPSV |
| | LFLVATA | VLMKPGASV | CLVKDYFPEPVTVSWNSGALTSGVHT | LFLVATA | SLSASLGDR | FIFPPSDE |
| | TGVHS | KLSCKATGY | FPAVLQSSGLYSLSSVVTVPSSSLGT | TGVHS | VTISCRASQ | QLKSGTAS |
| | (SEQ ID | TITGSWIAW | QTYICNVNHKPSNTKVDKKVEPKSCD | (SEQ ID | DISNYLNWY | VVCLLNNF |
| | NO: | LKQRPGHGL | KTHTCPPCPAPDLLGDDSVFLFPPKP | NO: | QQKPDGTVK | YPREAKVQ |
| | 588) | EWIGEILPG | KDTLMISRTPEVTCVVVDVSDEDGEV | 588) | LLIYYTSRL | WKVDNALQ |
| | | SGRINLNED | KFNWYVDGVEVHNAKTKPREEQYNST | | HSGVPSRES | SGNSQESV |
| | | FKGKATFTA | YRVVSVLTVLHQDWLNGKEYKCKVSN | | GSGSGTDYS | TEQDSKDS |
| | | DTSSNTVFI | KALPRPIEKTISKAKGQPREPQVYTL | | LTIRNLDQE | TYSLSSTL |
| | | QLSSLTTED | PPSRDELTKNQVSLTCLVKGFYPSDI | | DIATYFCQQ | TLSKADYE |
| | | SAIYYCYNG | AVEWESNGQPENNYKTTPPVLDSDGS | | DKTFPWTFG | KHKVYACE |
| | | SAFDYWGQG | FFLYSKLTVDKSRWQQGNVFSCSVMH | | GGTKLEIK | VTHQGLSS |
| | | TTLTVSS | EALHNHYTQKSLSLSPG (SEQ ID | | (SEQ ID | PVTKSFNR |
| | | (SEQ ID | NO: 587) | | NO: 414) | GEC (SEQ |
| | | NO: 322) | | | | ID NO: |
| | | | | | | 415) |

In some embodiments, non-limiting example of molecules comprising an anti-PD-1 antibody, or an antigen-binding fragment thereof, include those set forth in Table 11 below. In some embodiments, a bispecific antibody comprising an anti-PD-1 antibody and an FcγRIIβ-selective moiety is provided. In some embodiments, a bispecific antibody comprising an anti-PD-1 antibody in Fab format and a FcγRIIβ-selective moiety is provided. In some embodiments, a bispecific antibody comprising an anti-PD-1 antibody in scFv format and a FcγRIIβ-selective moiety is provided. In some embodiments, a bispecific antibody comprises an anti-PD-1 antibody, and a FcγRIIβ-selective Fc molecule. In some embodiments, a bispecific antibody comprises an anti-PD-1 antibody in Fab format, a and a FcγRIIβ-selective Fc molecule. In some embodiments, a bispecific antibody comprises an anti-PD-1 antibody in scFv format, and a FcγRIIβ-selective Fc molecule. In some embodiments, the bispecific antibody is as provided in Table 11.

TABLE 11

| ID# | VH Sequence | Fc Sequence | VL Sequence | Ck Sequence |
|---|---|---|---|---|
| TA313 | EVQLLESGGGLV | ASTKGPSVFPLAPSSKSTSGGTAALGCLVK | EIVMTQSPATL | RTVAAPSVFIF |
| | QPGGSLRLSCAA | DYFPEPVTVSWNSGALTSGVHTFPAVLQSS | SVSPGERATLS | PPSDEQLKSGT |
| | SGFTFSSDAMNW | GLYSLSSVVTVPSSSLGTQTYICNVNHKPS | CRASQSVSSNL | ASVVCLLNNFY |
| | VRQAPGKGLEWV | NTKVDKKVEPKSCDKTHTCPPCPAPELLGE | AWYQQKPGQAP | PREAKVQWKVD |
| | STIYSGGSTYYA | ESVFLFPPKPKDTLMISRTPEVTCVVVDVS | RLLIYGASTRA | NALQSGNSQES |
| | DSVKGRFTISRD | HEDPEVKFNWYVDGVEVHNAKTKPREEQYN | TGIPARFSGSG | VTEQDSKDSTY |
| | NSKNTLYLQMNS | STYRVVSVLTVLHQDWLNGKEYKCKVSNKA | SGTEFTLTISS | SLSSTLTLSKA |
| | LRAEDTAVYYCA | LPAPIEKTISKAKGQPREPQVYTLPPSRDE | LQSEDFAVYYC | DYEKHKVYACE |
| | RGGNFYNYFDYW | LTKNQVSLTCLVKGFYPSDIAVEWESNGQP | QQYNNWPPWTF | VTHQGLSSPVT |
| | GQGTLVTVSS | ENNYKTTPPVLDSDGSFFLYSKLTVDKSRW | GQGTKVEIK | KSFNRGEC |
| | (SEQ ID NO: | QQGNVFSCSVMHEALHNHYTQKSLSLSPG | (SEQ ID NO: | (SEQ ID NO: |
| | 136) | (SEQ ID NO: 543) | 323) | 415) |
| TA314 | EVQLLESGGGLV | ASTKGPSVFPLAPSSKSTSGGTAALGCLVK | EIVMTQSPATL | RTVAAPSVFIF |
| | QPGGSLRLSCAA | DYFPEPVTVSWNSGALTSGVHTFPAVLQSS | SVSPGERATLS | PPSDEQLKSGT |
| | SGFTFSSDAMNW | GLYSLSSVVTVPSSSLGTQTYICNVNHKPS | CRASQSVSSNL | ASVVCLLNNFY |
| | VRQAPGKGLEWV | NTKVDKKVEPKSCDKTHTCPPCPAPELLGD | AWYQQKPGQAP | PREAKVQWKVD |
| | STIYSGGSTYYA | GSAFLFPPKPKDTLMISRTPEVTCVVVDVS | RLLIYGASTRA | NALQSGNSQES |
| | DSVKGRFTISRD | HDEPEVKFNWYVDGVEVHNAKTKPREEQYN | TGIPARFSGSG | VTEQDSKDSTY |
| | NSKNTLYLQMNS | STYRVVSVLTVLHQDWLNGKEYKCKVSNKA | SGTEFTLTISS | SLSSTLTLSKA |
| | LRAEDTAVYYCA | LPRPIEKTISKAKGQPREPQVYTLPPSRDE | LQSEDFAVYYC | DYEKHKVYACE |
| | RGGNFYNYFDYW | LTKNQVSLTCLVKGFYPSDIAVEWESNGQP | QQYNNWPPWTF | VTHQGLSSPVT |
| | GQGTLVTVSS | ENNYKTTPPVLDSDGSFFLYSKLTVDKSRW | GQGTKVEIK | KSFNRGEC |
| | (SEQ ID NO: | QQGNVFSCSVMHEALHNHYTQKSLSLSPG | (SEQ ID NO: | (SEQ ID NO: |
| | 136) | (SEQ ID NO: 544) | 323) | 415) |
| TA315 | EVQLLESGGGLV | ASTKGPSVFPLAPSSKSTSGGTAALGCLVK | EIVMTQSPATL | RTVAAPSVFIF |
| | QPGGSLRLSCAA | DYFPEPVTVSWNSGALTSGVHTFPAVLQSS | SVSPGERATLS | PPSDEQLKSGT |
| | SGFTFSSDAMNW | GLYSLSSVVTVPSSSLGTQTYICNVNHKPS | CRASQSVSSNL | ASVVCLLNNFY |
| | VRQAPGKGLEWV | NTKVDKKVEPKSCDKTHTCPPCPAPEFEGG | AWYQQKPGQAP | PREAKVQWKVD |
| | STIYSGGSTYYA | PSVFLFPPKPKDTLMISRTPEVTCVVVDVS | RLLIYGASTRA | NALQSGNSQES |
| | DSVKGRFTISRD | DEDGEVKFNWYVDGVEVHNAKTKPREEQYN | TGIPARFSGSG | VTEQDSKDSTY |
| | NSKNTLYLQMNS | STYRVVSVLTVLHQDWLNGKEYKCKVSNKA | SGTEFTLTISS | SLSSTLTLSKA |
| | LRAEDTAVYYCA | LAAPIEKTISKAKGQPREPQVYTLPPSRDE | LQSEDFAVYYC | DYEKHKVYACE |
| | RGGNFYNYFDYW | LTKNQVSLTCLVKGFYPSDIAVEWESNGQP | QQYNNWPPWTF | VTHQGLSSPVT |
| | GQGTLVTVSS | ENNYKTTPPVLDSDGSFFLYSKLTVDKSRW | GQGTKVEIK | KSFNRGEC |
| | (SEQ ID NO: | QQGNVFSCSVMHEALHNHYTQKSLSLSPG | (SEQ ID NO: | (SEQ ID NO: |
| | 136) | (SEQ ID NO: 545) | 323) | 415) |
| TA316 | EVQLLESGGGLV | ASTKGPSVFPLAPSSKSTSGGTAALGCLVK | EIVMTQSPATL | RTVAAPSVFIF |
| | QPGGSLRLSCAA | DYFPEPVTVSWNSGALTSGVHTFPAVLQSS | SVSPGERATLS | PPSDEQLKSGT |
| | SGFTFSDHYMSW | GLYSLSSVVTVPSSSLGTQTYICNVNHKPS | CRASQSVSSNL | ASVVCLLNNFY |
| | VRQAPGKGLEWV | NTKVDKKVEPKSCDKTHTCPPCPAPELLGE | AWYQQKPGQAP | PREAKVQWKVD |
| | STISSGGNYIYY | ESVFLFPPKPKDTLMISRTPEVTCVVVDVS | RLLIYGASTRA | NALQSGNSQES |
| | ADSVKGRFTISR | HEDPEVKFNWYVDGVEVHNAKTKPREEQYN | TGIPARFSGSG | VTEQDSKDSTY |
| | DSSKNTLYLQMN | STYRVVSVLTVLHQDWLNGKEYKCKVSNKA | SGTEFTLTISS | SLSSTLTLSKA |
| | SLRAEDTAVYYC | LPAPIEKTISKAKGQPREPQVYTLPPSRDE | LQSEDFAVYYC | DYEKHKVYACE |
| | ARILKNGKYIYY | LTKNQVSLTCLVKGFYPSDIAVEWESNGQP | QQYNNWPPWTF | VTHQGLSSPVT |
| | FDYWGQGTLVTV | ENNYKTTPPVLDSDGSFFLYSKLTVDKSRW | GQGTKVEIK | KSFNRGEC |
| | SS (SEQ ID | QQGNVFSCSVMHEALHNHYTQKSLSLSPG | (SEQ ID NO: | (SEQ ID NO: |
| | NO: 156) | (SEQ ID NO: 543) | 323) | 415) |
| TA317 | EVQLLESGGGLV | ASTKGPSVFPLAPSSKSTSGGTAALGCLVK | EIVMTQSPATL | RTVAAPSVFIF |
| | QPGGSLRLSCAA | DYFPEPVTVSWNSGALTSGVHTFPAVLQSS | SVSPGERATLS | PPSDEQLKSGT |
| | SGFTFSDHYMSW | GLYSLSSVVTVPSSSLGTQTYICNVNHKPS | CRASQSVSSNL | ASVVCLLNNFY |
| | VRQAPGKGLEWV | NTKVDKKVEPKSCDKTHTCPPCPAPELLGD | AWYQQKPGQAP | PREAKVQWKVD |
| | STISSGGNYIYY | GSAFLFPPKPKDTLMISRTPEVTCVVVDVS | RLLIYGASTRA | NALQSGNSQES |
| | ADSVKGRFTISR | HDEPEVKFNWYVDGVEVHNAKTKPREEQYN | TGIPARFSGSG | VTEQDSKDSTY |
| | DSSKNTLYLQMN | STYRVVSVLTVLHQDWLNGKEYKCKVSNKA | SGTEFTLTISS | SLSSTLTLSKA |
| | SLRAEDTAVYYC | LPRPIEKTISKAKGQPREPQVYTLPPSRDE | LQSEDFAVYYC | DYEKHKVYACE |
| | ARILKNGKYIYY | LTKNQVSLTCLVKGFYPSDIAVEWESNGQP | QQYNNWPPWTF | VTHQGLSSPVT |
| | FDYWGQGTLVTV | ENNYKTTPPVLDSDGSFFLYSKLTVDKSRW | GQGTKVEIK | KSFNRGEC |
| | SS (SEQ ID | QQGNVFSCSVMHEALHNHYTQKSLSLSPG | (SEQ ID NO: | (SEQ ID NO: |
| | NO: 156) | (SEQ ID NO: 544) | 323) | 415) |
| TA318 | EVQLLESGGGLV | ASTKGPSVFPLAPSSKSTSGGTAALGCLVK | EIVMTQSPATL | RTVAAPSVFIF |
| | QPGGSLRLSCAA | DYFPEPVTVSWNSGALTSGVHTFPAVLQSS | SVSPGERATLS | PPSDEQLKSGT |
| | SGFTFSDHYMSW | GLYSLSSVVTVPSSSLGTQTYICNVNHKPS | CRASQSVSSNL | ASVVCLLNNFY |
| | VRQAPGKGLEWV | NTKVDKKVEPKSCDKTHTCPPCPAPEFEGG | AWYQQKPGQAP | PREAKVQWKVD |
| | STISSGGNYIYY | PSVFLFPPKPKDTLMISRTPEVTCVVVDVS | RLLIYGASTRA | NALQSGNSQES |
| | ADSVKGRFTISR | DEDGEVKFNWYVDGVEVHNAKTKPREEQYN | TGIPARFSGSG | VTEQDSKDSTY |
| | DSSKNTLYLQMN | STYRVVSVLTVLHQDWLNGKEYKCKVSNKA | SGTEFTLTISS | SLSSTLTLSKA |
| | SLRAEDTAVYYC | LAAPIEKTISKAKGQPREPQVYTLPPSRDE | LQSEDFAVYYC | DYEKHKVYACE |
| | ARILKNGKYIYY | LTKNQVSLTCLVKGFYPSDIAVEWESNGQP | QQYNNWPPWTF | VTHQGLSSPVT |
| | FDYWGQGTLVTV | ENNYKTTPPVLDSDGSFFLYSKLTVDKSRW | GQGTKVEIK | KSFNRGEC |
| | SS (SEQ ID | QQGNVFSCSVMHEALHNHYTQKSLSLSPG | (SEQ ID NO: | (SEQ ID NO: |
| | NO: 156) | (SEQ ID NO: 545) | 323) | 415) |
| TA319 | EVQLLESGGAFV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGNSY | KATLVCLISDE |
| | VRQAPGKGLEWV | NTKVDKKVEPKSCDKTHTCPPCPAPELLGE | VSWYQQLPGTA | YPGAVTVAWKA |
| | SVISNSGGSTYY | ESVFLFPPKPKDTLMISRTPEVTCVVVDVS | PKLLIYLNSQR | DSSPVKAGVET |
| | TDSVKGRFTISR | HEDPEVKFNWYVDGVEVHNAKTKPREEQYN | PSGVPDRFSGS | TTPSKQSNNKY |
| | DNSKNTLYLQIN | STYRVVSVLTVLHQDWLNGKEYKCKVSNKA | KSGTSASLAIS | AASSYLSLTPE |
| | SLRAEDTAVYYC | LPAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 187) | (SEQ ID NO: 543) | 344) | |
| TA320 | EVQLLESGGAFV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGNSY | KATLVCLISDF |
| | VRQAPGKGLEWV | NTKVDKKVEPKSCDKTHTCPPCPAPELLGD | VSWYQQLPGTA | YPGAVTVAWKA |
| | SVISNSGGSTYY | GSAFLFPPKPKDTLMISRTPEVTCVVVDVS | PKLLIYLNSQR | DSSPVKAGVET |
| | TDSVKGRFTISR | HDEPEVKFNWYVDGVEVHNAKTKPREEQYN | PSGVPDRFSGS | TTPSKQSNNKY |
| | DNSKNTLYLQIN | STYRVVSVLTVLHQDWLNGKEYKCKVSNKA | KSGTSASLAIS | AASSYLSLTPE |
| | SLRAEDTAVYYC | LPRPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 187) | (SEQ ID NO: 544) | 344) | |
| TA321 | EVQLLESGGAFV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGNSY | KATLVCLISDE |
| | VRQAPGKGLEWV | NTKVDKKVEPKSCDKTHTCPPCPAPEFEGG | VSWYQQLPGTA | YPGAVTVAWKA |
| | SVISNSGGSTYY | PSVFLFPPKPKDTLMISRTPEVTCVVVDVS | PKLLIYLNSQR | DSSPVKAGVET |
| | TDSVKGRFTISR | DEDGEVKFNWYVDGVEVHNAKTKPREEQYN | PSGVPDRFSGS | TTPSKQSNNKY |
| | DNSKNTLYLQIN | STYRVVSVLTVLHQDWLNGKEYKCKVSNKA | KSGTSASLAIS | AASSYLSLTPE |
| | SLRAEDTAVYYC | LAAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 187) | (SEQ ID NO: 545) | 344) | |
| TA322 | QVTLKESGPGIL | ASTKGPSVFPLAPSSKSTSGGTAALGCLVK | QAVVTQESALT | RTVAAPSVFIF |
| | QPSQTLSLTCSF | DYFPEPVTVSWNSGALTSGVHTFPAVLQSS | TSPGETVTLTC | PPSDEQLKSGT |
| | SGFSLSTFGMGV | GLYSLSSVVTVPSSSLGTQTYICNVNHKPS | RSSTGAVTTSN | ASVVCLLNNFY |
| | GWIRQPSGKGLE | NTKVDKKVEPKSCDKTHTCPPCPAPELLGE | YANWVQEKPDH | PREAKVQWKVD |
| | WLAHIWWDDDKY | ESVFLFPPKPKDTLMISRTPEVTCVVVDVS | LFTGLIGGINN | NALQSGNSQES |
| | YNPALKSRLTIS | HEDPEVKFNWYVDGVEVHNAKTKPREEQYN | RAPGVPARFSG | VTEQDSKDSTY |
| | KDTSKNQVFLKI | STYRVVSVLTVLHQDWLNGKEYKCKVSNKA | SLIGDKAALTI | SLSSTLTLSKA |
| | ANVDTADTATYY | LPAPIEKTISKAKGQPREPQVYTLPPSRDE | TGAQTEDEAIY | DYEKHKVYACE |
| | CARIITTAWYFD | LTKNQVSLTCLVKGFYPSDIAVEWESNGQP | FCALWYSNHFI | VTHQGLSSPVT |
| | VWGTGTTVTVSS | ENNYKTTPPVLDSDGSFFLYSKLTVDKSRW | FGSGTKVTVL | KSFNRGEC |
| | (SEQ ID NO: | QQGNVFSCSVMHEALHNHYTQKSLSLSPG | (SEQ ID NO: | (SEQ ID NO: |
| | 290) | (SEQ ID NO: 543) | 388) | 415) |
| TA323 | QVTLKESGPGIL | ASTKGPSVFPLAPSSKSTSGGTAALGCLVK | QAVVIQESALT | RTVAAPSVFIF |
| | QPSQTLSLTCSF | DYFPEPVTVSWNSGALTSGVHTFPAVLQSS | TSPGETVTLTC | PPSDEQLKSGT |
| | SGFSLSTFGMGV | GLYSLSSVVTVPSSSLGTQTYICNVNHKPS | RSSTGAVTTSN | ASVVCLLNNFY |
| | GWIRQPSGKGLE | NTKVDKKVEPKSCDKTHTCPPCPAPELLGD | YANWVQEKPDH | PREAKVQWKVD |
| | WLAHIWWDDDKY | GSAFLFPPKPKDTLMISRTPEVTCVVVDVS | LFTGLIGGINN | NALQSGNSQES |
| | YNPALKSRLTIS | HDEPEVKFNWYVDGVEVHNAKTKPREEQYN | RAPGVPARFSG | VTEQDSKDSTY |
| | KDTSKNQVFLKI | STYRVVSVLTVLHQDWLNGKEYKCKVSNKA | SLIGDKAALTI | SLSSTLTLSKA |
| | ANVDTADTATYY | LPRPIEKTISKAKGQPREPQVYTLPPSRDE | TGAQTEDEAIY | DYEKHKVYACE |
| | CARIITTAWYFD | LTKNQVSLTCLVKGFYPSDIAVEWESNGQP | FCALWYSNHFI | VTHQGLSSPVT |
| | VWGTGTTVTVSS | ENNYKTTPPVLDSDGSFFLYSKLTVDKSRW | FGSGTKVTVL | KSFNRGEC |
| | (SEQ ID NO: | QQGNVFSCSVMHEALHNHYTQKSLSLSPG | (SEQ ID NO: | (SEQ ID NO: |
| | 290) | (SEQ ID NO: 544) | 388) | 415) |
| TA324 | QVTLKESGPGIL | ASTKGPSVFPLAPSSKSTSGGTAALGCLVK | QAVVTQESALT | RTVAAPSVFIF |
| | QPSQTLSLTCSF | DYFPEPVTVSWNSGALTSGVHTFPAVLQSS | TSPGETVTLTC | PPSDEQLKSGT |
| | SGFSLSTFGMGV | GLYSLSSVVTVPSSSLGTQTYICNVNHKPS | RSSTGAVTTSN | ASVVCLLNNFY |
| | GWIRQPSGKGLE | NTKVDKKVEPKSCDKTHTCPPCPAPEFEGG | YANWVQEKPDH | PREAKVQWKVD |
| | WLAHIWWDDDKY | PSVFLFPPKPKDTLMISRTPEVTCVVVDVS | LFTGLIGGTNN | NALQSGNSQES |
| | YNPALKSRLTIS | DEDGEVKFNWYVDGVEVHNAKTKPREEQYN | RAPGVPARFSG | VTEQDSKDSTY |
| | KDTSKNQVFLKI | STYRVVSVLTVLHQDWLNGKEYKCKVSNKA | SLIGDKAALTI | SLSSTLTLSKA |
| | ANVDTADTATYY | LAAPIEKTISKAKGQPREPQVYTLPPSRDE | TGAQTEDEAIY | DYEKHKVYACE |
| | CARIITTAWYFD | LTKNQVSLTCLVKGFYPSDIAVEWESNGQP | FCALWYSNHFI | VTHQGLSSPVT |
| | VWGTGTTVTVSS | ENNYKTTPPVLDSDGSFFLYSKLTVDKSRW | FGSGTKVTVL | KSFNRGEC |
| | (SEQ ID NO: | QQGNVFSCSVMHEALHNHYTQKSLSLSPG | (SEQ ID NO: | (SEQ ID NO: |
| | 290) | (SEQ ID NO: 545) | 388) | 415) |
| TA325 | QVTLKESGPGIL | ASTKGPSVFPLAPSSKSTSGGTAALGCLVK | QAVVTQESALT | RTVAAPSVFIF |
| | QPSQTLSLTCSF | DYFPEPVTVSWNSGALTSGVHTFPAVLQSS | TSPGETVTLTC | PPSDEQLKSGT |
| | SGFSLSTFGMGV | GLYSLSSVVTVPSSSLGTQTYICNVNHKPS | RSSTGAVTTSN | ASVVCLLNNFY |
| | GWIRQPSGKGLE | NTKVDKKVEPKSCDKTHTCPPCPAPELLGG | YANWVQEKPDH | PREAKVQWKVD |
| | WLAHIWWDDDKY | PSVFLFPPKPKDTLMISRTPEVTCVVVDVS | LFTGLIGGINN | NALQSGNSQES |
| | YNPALKSRLTIS | HEDPEVKFNWYVDGVEVHNAKTKPREEQYN | RAPGVPARFSG | VTEQDSKDSTY |
| | KDTSKNQVFLKI | STYRVVSVLTVLHQDWLNGKEYKCKVSNKA | SLIGDKAALTI | SLSSTLTLSKA |
| | ANVDTADTATYY | LPAPIEKTISKAKGQPREPQVYTLPPSRDE | TGAQTEDEAIY | DYEKHKVYACE |
| | CARIITTAWYFD | LTKNQVSLTCLVKGFYPSDIAVEWESNGQP | FCALWYSNHFI | VTHQGLSSPVT |
| | VWGTGTTVTVSS | ENNYKTTPPVLDSDGSFFLYSKLTVDKSRW | FGSGTKVTVL | KSFNRGEC |
| | (SEQ ID NO: | QQGNVFSCSVMHEALHNHYTQKSLSLSPG | (SEQ ID NO: | (SEQ ID NO: |
| | 290) | (SEQ ID NO: 546) | 388) | 415) |
| TA326 | EVQLLESGGGLV | ASTKGPSVFPLAPSSKSTSGGTAALGCLVK | EIVMTQSPATL | RTVAAPSVFIF |
| | QPGGSLRLSCAA | DYFPEPVTVSWNSGALTSGVHTFPAVLQSS | SVSPGERATLS | PPSDEQLKSGT |
| | SGFTFSSDAMNW | GLYSLSSVVTVPSSSLGTQTYICNVNHKPS | CRASQSVSSNL | ASVVCLLNNFY |
| | VRQAPGKGLEWV | NTKVDKKVEPKSCDKTHTCPPCPAPELLGG | AWYQQKPGQAP | PREAKVQWKVD |
| | STIYSGGSTYYA | PSVFLFPPKPKDTLMISRTPEVTCVVVDVS | RLLIYGASTRA | NALQSGNSQES |
| | DSVKGRFTISRD | HEDPEVKFNWYVDGVEVHNAKTKPREEQYN | TGIPARFSGSG | VTEQDSKDSTY |
| | NSKNTLYLQMNS | STYRVVSVLTVLHQDWLNGKEYKCKVSNKA | SGTEFTLTISS | SLSSTLTLSKA |
| | LRAEDTAVYYCA | LPAPIEKTISKAKGQPREPQVYTLPPSRDE | LQSEDFAVYYC | DYEKHKVYACE |
| | RGGNFYNYFDYW | LTKNQVSLTCLVKGFYPSDIAVEWESNGQP | QQYNNWPPWTF | VTHQGLSSPVT |
| | GQGTLVTVSS | ENNYKTTPPVLDSDGSFFLYSKLTVDKSRW | GQGTKVEIK | KSFNRGEC |
| | (SEQ ID NO: | QQGNVFSCSVMHEALHNHYTQKSLSLSPG | (SEQ ID NO: | (SEQ ID NO: |
| | 136) | (SEQ ID NO: 546) | 323) | 415) |
| TA327 | EVQLLESGGGLV | ASTKGPSVFPLAPSSKSTSGGTAALGCLVK | EIVMTQSPATL | RTVAAPSVFIF |
| | QPGGSLRLSCAA | DYFPEPVTVSWNSGALTSGVHTFPAVLQSS | SVSPGERATLS | PPSDEQLKSGT |
| | SGFTFSDHYMSW | GLYSLSSVVTVPSSSLGTQTYICNVNHKPS | CRASQSVSSNL | ASVVCLLNNFY |
| | VRQAPGKGLEWV | NTKVDKKVEPKSCDKTHTCPPCPAPELLGG | AWYQQKPGQAP | PREAKVQWKVD |
| | STISSGGNYIYY | PSVFLFPPKPKDTLMISRTPEVTCVVVDVS | RLLIYGASTRA | NALQSGNSQES |
| | ADSVKGRFTISR | HEDPEVKFNWYVDGVEVHNAKTKPREEQYN | TGIPARFSGSG | VTEQDSKDSTY |
| | DSSKNTLYLQMN | STYRVVSVLTVLHQDWLNGKEYKCKVSNKA | SGTEFTLTISS | SLSSTLTLSKA |
| | SLRAEDTAVYYC | LPAPIEKTISKAKGQPREPQVYTLPPSRDE | LQSEDFAVYYC | DYEKHKVYACE |
| | ARILKNGKYIYY | LTKNQVSLTCLVKGFYPSDIAVEWESNGQP | QQYNNWPPWTF | VTHQGLSSPVT |
| | FDYWGQGTLVTV | ENNYKTTPPVLDSDGSFFLYSKLTVDKSRW | GQGTKVEIK | KSFNRGEC |
| | SS (SEQ ID | QQGNVFSCSVMHEALHNHYTQKSLSLSPG | (SEQ ID NO: | (SEQ ID NO: |
| | NO: 156) | (SEQ ID NO: 546) | 323) | 415) |
| TA328 | EVQLLESGGAFV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGNSY | KATLVCLISDE |
| | VRQAPGKGLEWV | NTKVDKKVEPKSCDKTHTCPPCPAPELLGG | VSWYQQLPGTA | YPGAVTVAWKA |
| | SVISNSGGSTYY | PSVFLFPPKPKDTLMISRTPEVTCVVVDVS | PKLLIYLNSQR | DSSPVKAGVET |
| | TDSVKGRFTISR | HEDPEVKFNWYVDGVEVHNAKTKPREEQYN | PSGVPDRFSGS | TTPSKQSNNKY |
| | DNSKNTLYLQIN | STYRVVSVLTVLHQDWLNGKEYKCKVSNKA | KSGTSASLAIS | AASSYLSLTPE |
| | SLRAEDTAVYYC | LPAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 187) | (SEQ ID NO: 546) | 344) | |
| TA329 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGESY | KATLVCLISDE |
| | VRQAPGKGLEWV | NTKVDKKVEPKSCDKTHTCPPCPAPELLGE | VSWYQQLPGTA | YPGAVTVAWKA |
| | SVISNSGGSTYY | ESVFLFPPKPKDTLMISRTPEVTCVVVDVS | PKLLIYLNSQR | DSSPVKAGVET |
| | TDSVKGRFTISR | HEDPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 543) | 531) | |
| TA330 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGGSY | KATLVCLISDE |
| | VRQAPGKGLEWV | NTKVDKKVEPKSCDKTHTCPPCPAPELLGE | VSWYQQLPGTA | YPGAVTVAWKA |
| | SVISNSGGSTYY | ESVFLFPPKPKDTLMISRTPEVTCVVVDVS | PKLLIYLNSQR | DSSPVKAGVET |
| | TDSVKGRFTISR | HEDPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 543) | 532) | |
| TA331 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGLSY | KATLVCLISDF |
| | VRQAPGKGLEWV | NTKVDKKVEPKSCDKTHTCPPCPAPELLGE | VSWYQQLPGTA | YPGAVTVAWKA |
| | SVISNSGGSTYY | ESVFLFPPKPKDTLMISRTPEVTCVVVDVS | PKLLIYLNSQR | DSSPVKAGVET |
| | TDSVKGRFTISR | HEDPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 543) | 533) | |
| TA332 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGQSY | KATLVCLISDE |
| | VRQAPGKGLEWV | NTKVDKKVEPKSCDKTHTCPPCPAPELLGE | VSWYQQLPGTA | YPGAVTVAWKA |
| | SVISNSGGSTYY | ESVFLFPPKPKDTLMISRTPEVTCVVVDVS | PKLLIYLNSQR | DSSPVKAGVET |
| | TDSVKGRFTISR | HEDPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 543) | 534) | |
| TA333 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGSSY | KATLVCLISDE |
| | VRQAPGKGLEWV | NTKVDKKVEPKSCDKTHTCPPCPAPELLGE | VSWYQQLPGTA | YPGAVTVAWKA |
| | SVISNSGGSTYY | ESVFLFPPKPKDTLMISRTPEVTCVVVDVS | PKLLIYLNSQR | DSSPVKAGVET |
| | TDSVKGRFTISR | HEDPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 543) | 535) | |
| TA334 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGNDY | KATLVCLISDE |
| | VRQAPGKGLEWV | NTKVDKKVEPKSCDKTHTCPPCPAPELLGE | VSWYQQLPGTA | YPGAVTVAWKA |
| | SVISNSGGSTYY | ESVFLFPPKPKDTLMISRTPEVTCVVVDVS | PKLLIYLNSQR | DSSPVKAGVET |
| | TDSVKGRFTISR | HEDPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID: | ID NO: 415) |
| | 530) | (SEQ ID NO: 543) | 536) | |
| TA335 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGNQY | KATLVCLISDE |
| | VRQAPGKGLEWV | NTKVDKKVEPKSCDKTHTCPPCPAPELLGE | VSWYQQLPGTA | YPGAVTVAWKA |
| | SVISNSGGSTYY | ESVFLFPPKPKDTLMISRTPEVTCVVVDVS | PKLLIYLNSQR | DSSPVKAGVET |
| | TDSVKGRFTISR | HEDPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 543) | 537) | |
| TA336 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGESY | KATLVCLISDF |
| | VRQAPGKGLEWV | NTKVDKKVEPKSCDKTHTCPPCPAPELLGD | VSWYQQLPGTA | YPGAVTVAWKA |
| | SVISNSGGSTYY | GSAFLFPPKPKDTLMISRTPEVTCVVVDVS | PKLLIYLNSQR | DSSPVKAGVET |
| | TDSVKGRFTISR | HDEPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPRPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 544) | 531) | |
| TA337 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGGSY | KATLVCLISDF |
| | VRQAPGKGLEWV | NTKVDKKVEPKSCDKTHTCPPCPAPELLGD | VSWYQQLPGTA | YPGAVTVAWKA |
| | SVISNSGGSTYY | GSAFLFPPKPKDTLMISRTPEVTCVVVDVS | PKLLIYLNSQR | DSSPVKAGVET |
| | TDSVKGRFTISR | HDEPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPRPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 544) | 532) | |
| TA338 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGLSY | KATLVCLISDE |
| | VRQAPGKGLEWV | NTKVDKKVEPKSCDKTHTCPPCPAPELLGD | VSWYQQLPGTA | YPGAVTVAWKA |
| | SVISNSGGSTYY | GSAFLFPPKPKDTLMISRTPEVTCVVVDVS | PKLLIYLNSQR | DSSPVKAGVET |
| | TDSVKGRFTISR | HDEPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPRPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 544) | 533) | |
| TA339 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGQSY | KATLVCLISDE |
| | VRQAPGKGLEWV | NTKVDKKVEPKSCDKTHTCPPCPAPELLGD | VSWYQQLPGTA | YPGAVTVAWKA |
| | SVISNSGGSTYY | GSAFLFPPKPKDTLMISRTPEVTCVVVDVS | PKLLIYLNSQR | DSSPVKAGVET |
| | TDSVKGRFTISR | HDEPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPRPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 544) | 534) | |
| TA340 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGSSY | KATLVCLISDE |
| | VRQAPGKGLEWV | NTKVDKKVEPKSCDKTHTCPPCPAPELLGD | VSWYQQLPGTA | YPGAVTVAWKA |
| | SVISNSGGSTYY | GSAFLFPPKPKDTLMISRTPEVTCVVVDVS | PKLLIYLNSQR | DSSPVKAGVET |
| | TDSVKGRFTISR | HDEPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPRPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 544) | 535) | |
| TA341 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGNDY | KATLVCLISDE |
| | VRQAPGKGLEWV | NTKVDKKVEPKSCDKTHTCPPCPAPELLGD | VSWYQQLPGTA | YPGAVTVAWKA |
| | SVISNSGGSTYY | GSAFLFPPKPKDTLMISRTPEVTCVVVDVS | PKLLIYLNSQR | DSSPVKAGVET |
| | TDSVKGRFTISR | HDEPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPRPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 544) | 536) | |
| TA342 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGNQY | KATLVCLISDE |
| | VRQAPGKGLEWV | NTKVDKKVEPKSCDKTHTCPPCPAPELLGD | VSWYQQLPGTA | YPGAVTVAWKA |
| | SVISNSGGSTYY | GSAFLFPPKPKDTLMISRTPEVTCVVVDVS | PKLLIYLNSQR | DSSPVKAGVET |
| | TDSVKGRFTISR | HDEPEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LPRPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 544) | 537) | |
| TA343 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGESY | KATLVCLISDE |
| | VRQAPGKGLEWV | NTKVDKKVEPKSCDKTHTCPPCPAPEFEGG | VSWYQQLPGTA | YPGAVTVAWKA |
| | SVISNSGGSTYY | PSVFLFPPKPKDTLMISRTPEVTCVVVDVS | PKLLIYLNSQR | DSSPVKAGVET |
| | TDSVKGRFTISR | DEDGEVKENWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LAAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 545) | 531) | |
| TA344 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGGSY | KATLVCLISDE |
| | VRQAPGKGLEWV | NTKVDKKVEPKSCDKTHTCPPCPAPEFEGG | VSWYQQLPGTA | YPGAVTVAWKA |
| | SVISNSGGSTYY | PSVFLFPPKPKDTLMISRTPEVTCVVVDVS | PKLLIYLNSQR | DSSPVKAGVET |
| | TDSVKGRFTISR | DEDGEVKFNWYVDGVEVHNAKTKPREEQYN | PSGVPDRFSGS | TTPSKQSNNKY |
| | DNSKNTLYLQMN | STYRVVSVLIVLHQDWLNGKEYKCKVSNKA | KSGTSASLAIS | AASSYLSLTPE |
| | SLRAEDTAVYYC | LAAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 545) | 532) | |
| TA345 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGLSY | KATLVCLISDF |
| | VRQAPGKGLEWV | NTKVDKKVEPKSCDKTHTCPPCPAPEFEGG | VSWYQQLPGTA | YPGAVTVAWKA |
| | SVISNSGGSTYY | PSVFLFPPKPKDTLMISRTPEVTCVVVDVS | PKLLIYLNSQR | DSSPVKAGVET |
| | TDSVKGRFTISR | DEDGEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LAAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 545) | 533) | |
| TA346 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGQSY | KATLVCLISDE |
| | VRQAPGKGLEWV | NTKVDKKVEPKSCDKTHTCPPCPAPEFEGG | VSWYQQLPGTA | YPGAVTVAWKA |
| | SVISNSGGSTYY | PSVFLFPPKPKDTLMISRTPEVTCVVVDVS | PKLLIYLNSQR | DSSPVKAGVET |
| | TDSVKGRFTISR | DEDGEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LAAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 545) | 534) | |
| TA347 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGSSY | KATLVCLISDF |
| | VRQAPGKGLEWV | NTKVDKKVEPKSCDKTHTCPPCPAPEFEGG | VSWYQQLPGTA | YPGAVTVAWKA |
| | SVISNSGGSTYY | PSVFLFPPKPKDTLMISRTPEVTCVVVDVS | PKLLIYLNSQR | DSSPVKAGVET |
| | TDSVKGRFTISR | DEDGEVKENWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LAAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 545) | 535) | |
| TA348 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGNDY | KATLVCLISDF |
| | VRQAPGKGLEWV | NTKVDKKVEPKSCDKTHTCPPCPAPEFEGG | VSWYQQLPGTA | YPGAVTVAWKA |
| | SVISNSGGSTYY | PSVFLFPPKPKDTLMISRTPEVTCVVVDVS | PKLLIYLNSQR | DSSPVKAGVET |
| | TDSVKGRFTISR | DEDGEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LAAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 545) | 536) | |
| TA349 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGNQY | KATLVCLISDE |
| | VRQAPGKGLEWV | NTKVDKKVEPKSCDKTHTCPPCPAPEFEGG | VSWYQQLPGTA | YPGAVTVAWKA |
| | SVISNSGGSTYY | PSVFLFPPKPKDTLMISRTPEVTCVVVDVS | PKLLIYLNSQR | DSSPVKAGVET |
| | TDSVKGRFTISR | DEDGEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LAAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 545) | 537) | |
| TA350 | QVTLKESGPTLV | ASTKGPSVFPLAPSSKSTSGGTAALGCLVK | QAVVTQEPSLT | GQPKAAPSVTL |
| | KPTQTLTLTCTF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKALE | NTKVDKKVEPKSCDKTHTCPPCPAPELLGE | YANWVQQKPGQ | YPGAVTVAWKA |
| | WLAHIWWDDDKY | ESVFLFPPKPKDTLMISRTPEVTCVVVDVS | AFRGLIGGINN | DSSPVKAGVET |
| | YNPALKSRLTIT | HEDPEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SILGNKAALTI | AASSYLSLTPE |
| | TNMDPVDTATYY | LPAPIEKTISKAKGQPREPQVYTLPPSRDE | TGAQADDESIY | QWKSHRSYSCQ |
| | CARIITTAWYFD | LTKNQVSLTCLVKGFYPSDIAVEWESNGQP | FCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID | QQGNVFSCSVMHEALHNHYTQKSLSLSPG | (SEQ ID NO: | ID NO: 415) |
| | NO: 538) | (SEQ ID NO: 543) | 539) | |
| TA351 | QVTLKESGPTLV | ASTKGPSVFPLAPSSKSTSGGTAALGCLVK | QAVVTQEPSLT | GQPKAAPSVTL |
| | KPTQTLTLTCTF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKALE | NTKVDKKVEPKSCDKTHTCPPCPAPELLGD | YANWVQQKPGQ | YPGAVTVAWKA |
| | WLAHIWWDDDKY | GSAFLFPPKPKDTLMISRTPEVTCVVVDVS | AFRGLIGGTNN | DSSPVKAGVET |
| | YNPALKSRLTIT | HDEPEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SILGNKAALTI | AASSYLSLTPE |
| | TNMDPVDTATYY | LPRPIEKTISKAKGQPREPQVYTLPPSRDE | TGAQADDESIY | QWKSHRSYSCQ |
| | CARIITTAWYFD | LTKNQVSLTCLVKGFYPSDIAVEWESNGQP | FCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID NO: | QQGNVFSCSVMHEALHNHYTQKSLSLSPG | (SEQ ID NO: | ID NO: 415) |
| | 538) | (SEQ ID NO: 544) | 539) | |
| TA352 | QVTLKESGPTLV | ASTKGPSVFPLAPSSKSTSGGTAALGCLVK | QAVVTQEPSLT | GQPKAAPSVTL |
| | KPTQTLTLTCTF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKALE | NTKVDKKVEPKSCDKTHTCPPCPAPEFEGG | YANWVQQKPGQ | YPGAVTVAWKA |
| | WLAHIWWDDDKY | PSVFLFPPKPKDTLMISRTPEVTCVVVDVS | AFRGLIGGINN | DSSPVKAGVET |
| | YNPALKSRLTIT | DEDGEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SILGNKAALTI | AASSYLSLTPE |
| | TNMDPVDTATYY | LAAPIEKTISKAKGQPREPQVYTLPPSRDE | TGAQADDESIY | QWKSHRSYSCQ |
| | CARIITTAWYFD | LTKNQVSLTCLVKGFYPSDIAVEWESNGQP | FCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID NO: | QQGNVFSCSVMHEALHNHYTQKSLSLSPG | (SEQ ID NO: | ID NO: 415) |
| | 538) | (SEQ ID NO: 545) | 539) | |
| TA353 | QVTLKESGPTLV | ASTKGPSVFPLAPSSKSTSGGTAALGCLVK | QAVVTQEPSLT | GQPKAAPSVTL |
| | KPTQTLTLTCSF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKGLE | NTKVDKKVEPKSCDKTHTCPPCPAPELLGE | YANWVQQKPGQ | YPGAVTVAWKA |
| | WIGHIWWDDDKY | ESVFLFPPKPKDTLMISRTPEVTCVVVDVS | AFRGLIGGINN | DSSPVKAGVET |
| | YNPALKSRLTIT | HEDPEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SILGNKAALTI | AASSYLSLTPE |
| | TNMDPVDTATYY | LPAPIEKTISKAKGQPREPQVYTLPPSRDE | TGAQADDESIY | QWKSHRSYSCQ |
| | CARIITTAWYFD | LTKNQVSLTCLVKGFYPSDIAVEWESNGQP | FCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID NO: | QQGNVFSCSVMHEALHNHYTQKSLSLSPG | (SEQ ID NO: | ID NO: 415) |
| | 540) | (SEQ ID NO: 543) | 539) | |
| TA354 | QVTLKESGPTLV | ASTKGPSVFPLAPSSKSTSGGTAALGCLVK | QAVVTQEPSLT | GQPKAAPSVTL |
| | KPTQTLTLTCSF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKGLE | NTKVDKKVEPKSCDKTHTCPPCPAPELLGD | YANWVQQKPGQ | YPGAVTVAWKA |
| | WIGHIWWDDDKY | GSAFLFPPKPKDTLMISRTPEVTCVVVDVS | AFRGLIGGTNN | DSSPVKAGVET |
| | YNPALKSRLTIT | HDEPEVKFNWYVDGVEVHNAKTKPREEQYN | RAPGVPDRFSG | AASSYLSLTPE |
| | KDTSKNQVVLTM | STYRVVSVLTVLHQDWLNGKEYKCKVSNKA | SILGNKAALTI | QWKSHRSYSCQ |
| | TNMDPVDTATYY | LPRPIEKTISKAKGQPREPQVYTLPPSRDE | TGAQADDESIY | VTHEGSTVEKT |
| | CARIITTAWYED | LTKNQVSLTCLVKGFYPSDIAVEWESNGQP | FCALWYSNHFI | VAPTECS (SEQ |
| | VWGTGTTVTVSS | ENNYKTTPPVLDSDGSFFLYSKLTVDKSRW | FGSGTKVTVL | ID NO: 415) |
| | (SEQ ID NO: | QQGNVFSCSVMHEALHNHYTQKSLSLSPG | (SEQ ID NO: | |
| | 540) | (SEQ ID NO: 544) | 539) | |
| TA355 | QVTLKESGPTLV | ASTKGPSVFPLAPSSKSTSGGTAALGCLVK | QAVVTQEPSLT | GQPKAAPSVTL |
| | KPTQTLTLTCSF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKGLE | NTKVDKKVEPKSCDKTHTCPPCPAPEFEGG | YANWVQQKPGQ | YPGAVTVAWKA |
| | WIGHIWWDDDKY | PSVFLFPPKPKDTLMISRTPEVTCVVVDVS | AFRGLIGGTNN | DSSPVKAGVET |
| | YNPALKSRLTIT | DEDGEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SILGNKAALTI | AASSYLSLTPE |
| | TNMDPVDTATYY | LAAPIEKTISKAKGQPREPQVYTLPPSRDE | TGAQADDESIY | QWKSHRSYSCQ |
| | CARIITTAWYFD | LTKNQVSLTCLVKGFYPSDIAVEWESNGQP | FCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID NO: | QQGNVFSCSVMHEALHNHYTQKSLSLSPG | (SEQ ID NO: | ID NO: 415) |
| | 540) | (SEQ ID NO: 545) | 539) | |
| TA356 | QVTLKESGPTLV | ASTKGPSVFPLAPSSKSTSGGTAALGCLVK | QAVVTQEPSLT | GQPKAAPSVTL |
| | KPTQTLTLTCSF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKGLE | NTKVDKKVEPKSCDKTHTCPPCPAPELLGE | YANWVQQKPGQ | YPGAVTVAWKA |
| | WIGHIWWDDDKY | ESVFLFPPKPKDTLMISRTPEVTCVVVDVS | AFRGLIGGINN | DSSPVKAGVET |
| | YNPALKSRLTIT | HEDPEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SLIGDKAALTI | AASSYLSLTPE |
| | TNMDPVDTATYY | LPAPIEKTISKAKGQPREPQVYTLPPSRDE | TGAQADDESDY | QWKSHRSYSCQ |
| | CARIITTAWYFD | LTKNQVSLTCLVKGFYPSDIAVEWESNGQP | YCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID NO: | QQGNVFSCSVMHEALHNHYTQKSLSLSPG | (SEQ ID NO: | ID NO: 415) |
| | 540) | (SEQ ID NO: 543) | 541) | |
| TA357 | QVTLKESGPTLV | ASTKGPSVFPLAPSSKSTSGGTAALGCLVK | QAVVTQEPSLT | GQPKAAPSVTL |
| | KPTQTLTLTCSF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKGLE | NTKVDKKVEPKSCDKTHTCPPCPAPELLGD | YANWVQQKPGQ | YPGAVTVAWKA |
| | WIGHIWWDDDKY | GSAFLFPPKPKDTLMISRTPEVTCVVVDVS | AFRGLIGGTNN | DSSPVKAGVET |
| | YNPALKSRLTIT | HDEPEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SLIGDKAALTI | AASSYLSLTPE |
| | TNMDPVDTATYY | LPRPIEKTISKAKGQPREPQVYTLPPSRDE | TGAQADDESDY | QWKSHRSYSCQ |
| | CARIITTAWYFD | LTKNQVSLTCLVKGFYPSDIAVEWESNGQP | YCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID NO: | QQGNVFSCSVMHEALHNHYTQKSLSLSPG | (SEQ ID NO: | ID NO: 415) |
| | 540) | (SEQ ID NO: 544) | 541) | |
| TA358 | QVTLKESGPTLV | ASTKGPSVFPLAPSSKSTSGGTAALGCLVK | QAVVTQEPSLT | GQPKAAPSVTL |
| | KPTQTLTLTCSF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKGLE | NTKVDKKVEPKSCDKTHTCPPCPAPEFEGG | YANWVQQKPGQ | YPGAVTVAWKA |
| | WIGHIWWDDDKY | PSVFLFPPKPKDTLMISRTPEVTCVVVDVS | AFRGLIGGTNN | DSSPVKAGVET |
| | YNPALKSRLTIT | DEDGEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SLIGDKAALTI | AASSYLSLTPE |
| | TNMDPVDTATYY | LAAPIEKTISKAKGQPREPQVYTLPPSRDE | TGAQADDESDY | QWKSHRSYSCQ |
| | CARIITTAWYFD | LTKNQVSLTCLVKGFYPSDIAVEWESNGQP | YCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID NO: | QQGNVFSCSVMHEALHNHYTQKSLSLSPG | (SEQ ID NO: | ID NO: 415) |
| | 540) | (SEQ ID NO: 545) | 541) | |
| TA359 | QVTLKESGPTLV | ASTKGPSVFPLAPSSKSTSGGTAALGCLVK | QAVVTQEPSLT | GQPKAAPSVTL |
| | KPTQTLTLTCTF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKALE | NTKVDKKVEPKSCDKTHTCPPCPAPELLGE | YANWVQQKPGQ | YPGAVTVAWKA |
| | WLAHIWWDDDKY | ESVFLFPPKPKDTLMISRTPEVTCVVVDVS | AFRGLIGGTNN | DSSPVKAGVET |
| | YNPALKSRLTIT | HEDPEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SILGNKAALTI | AASSYLSLTPE |
| | TNMDPVDTATYY | LPAPIEKTISKAKGQPREPQVYTLPPSRDE | TGAQADDESDY | QWKSHRSYSCQ |
| | CARIITTAWYFD | LTKNQVSLTCLVKGFYPSDIAVEWESNGQP | YCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID NO: | QQGNVFSCSVMHEALHNHYTQKSLSLSPG | (SEQ ID NO: | ID NO: 415) |
| | 538) | (SEQ ID NO: 543) | 542) | |
| TA360 | QVTLKESGPTLV | ASTKGPSVFPLAPSSKSTSGGTAALGCLVK | QAVVTQEPSLT | GQPKAAPSVTL |
| | KPTQTLTLTCTF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKALE | NTKVDKKVEPKSCDKTHTCPPCPAPELLGD | YANWVQQKPGQ | YPGAVTVAWKA |
| | WLAHIWWDDDKY | GSAFLFPPKPKDTLMISRTPEVTCVVVDVS | AFRGLIGGINN | DSSPVKAGVET |
| | YNPALKSRLTIT | HDEPEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SILGNKAALTI | AASSYLSLTPE |
| | TNMDPVDTATYY | LPRPIEKTISKAKGQPREPQVYTLPPSRDE | TGAQADDESDY | QWKSHRSYSCQ |
| | CARIITTAWYFD | LTKNQVSLTCLVKGFYPSDIAVEWESNGQP | YCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID NO: | QQGNVFSCSVMHEALHNHYTQKSLSLSPG | (SEQ ID NO: | ID NO: 415) |
| | 538) | (SEQ ID NO: 544) | 542) | |
| TA361 | QVTLKESGPTLV | ASTKGPSVFPLAPSSKSTSGGTAALGCLVK | QAVVTQEPSLT | GQPKAAPSVTL |
| | KPTQTLTLTCTF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKALE | NTKVDKKVEPKSCDKTHTCPPCPAPEFEGG | YANWVQQKPGQ | YPGAVTVAWKA |
| | WLAHIWWDDDKY | PSVFLFPPKPKDTLMISRTPEVTCVVVDVS | AFRGLIGGINN | DSSPVKAGVET |
| | YNPALKSRLTIT | DEDGEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SILGNKAALTI | AASSYLSLTPE |
| | TNMDPVDTATYY | LAAPIEKTISKAKGQPREPQVYTLPPSRDE | TGAQADDESDY | QWKSHRSYSCQ |
| | CARIITTAWYFD | LTKNQVSLTCLVKGFYPSDIAVEWESNGQP | YCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID NO: | QQGNVFSCSVMHEALHNHYTQKSLSLSPG | (SEQ ID NO: | ID NO: 415) |
| | 538) | (SEQ ID NO: 545) | 542) | |
| TA362 | QVTLKESGPTLV | ASTKGPSVFPLAPSSKSTSGGTAALGCLVK | QAVVTQEPSLT | GQPKAAPSVTL |
| | KPTQTLTLTCSF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKGLE | NTKVDKKVEPKSCDKTHTCPPCPAPELLGE | YANWVQQKPGQ | YPGAVTVAWKA |
| | WIGHIWWDDDKY | ESVFLFPPKPKDTLMISRTPEVTCVVVDVS | AFRGLIGGTNN | DSSPVKAGVET |
| | YNPALKSRLTIT | HEDPEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SILGNKAALTI | AASSYLSLTPE |
| | TNMDPVDTATYY | LPAPIEKTISKAKGQPREPQVYTLPPSRDE | TGAQADDESDY | QWKSHRSYSCQ |
| | CARIITTAWYED | LTKNQVSLTCLVKGFYPSDIAVEWESNGQP | YCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID NO: | QQGNVFSCSVMHEALHNHYTQKSLSLSPG | (SEQ ID NO: | ID NO: 415) |
| | 540) | (SEQ ID NO: 543) | 542) | |
| TA363 | QVTLKESGPTLV | ASTKGPSVFPLAPSSKSTSGGTAALGCLVK | QAVVTQEPSLT | GQPKAAPSVTL |
| | KPTQTLTLTCSF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDF |
| | GWIRQPPGKGLE | NTKVDKKVEPKSCDKTHTCPPCPAPELLGD | YANWVQQKPGQ | YPGAVTVAWKA |
| | WIGHIWWDDDKY | GSAFLFPPKPKDTLMISRTPEVTCVVVDVS | AFRGLIGGTNN | DSSPVKAGVET |
| | YNPALKSRLTIT | HDEPEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SILGNKAALTI | AASSYLSLTPE |
| | TNMDPVDTATYY | LPRPIEKTISKAKGQPREPQVYTLPPSRDE | TGAQADDESDY | QWKSHRSYSCQ |
| | CARIITTAWYFD | LTKNQVSLTCLVKGFYPSDIAVEWESNGQP | YCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID NO: | QQGNVFSCSVMHEALHNHYTQKSLSLSPG | (SEQ ID NO: | ID NO: 415) |
| | 540) | (SEQ ID NO: 544) | 542) | |
| TA364 | QVTLKESGPTLV | ASTKGPSVFPLAPSSKSTSGGTAALGCLVK | QAVVTQEPSLT | GQPKAAPSVTL |
| | KPTQTLTLTCSF | DYFPEPVTVSWNSGALTSGVHTFPAVLQSS | VSPGGTVTLTC | FPPSSEELQAN |
| | SGFSLSTFGMGV | GLYSLSSVVTVPSSSLGTQTYICNVNHKPS | RSSTGAVTTSN | KATLVCLISDE |
| | GWIRQPPGKGLE | NTKVDKKVEPKSCDKTHTCPPCPAPEFEGG | YANWVQQKPGQ | YPGAVTVAWKA |
| | WIGHIWWDDDKY | PSVFLFPPKPKDTLMISRTPEVTCVVVDVS | AFRGLIGGINN | DSSPVKAGVET |
| | YNPALKSRLTIT | DEDGEVKFNWYVDGVEVHNAKTKPREEQYN | RAPGVPDRFSG | TTPSKQSNNKY |
| | KDTSKNQVVLTM | STYRVVSVLTVLHQDWLNGKEYKCKVSNKA | SILGNKAALTI | AASSYLSLTPE |
| | TNMDPVDTATYY | LAAPIEKTISKAKGQPREPQVYTLPPSRDE | TGAQADDESDY | QWKSHRSYSCQ |
| | CARIITTAWYFD | LTKNQVSLTCLVKGFYPSDIAVEWESNGQP | YCALWYSNHFI | VTHEGSTVEKT |
| | VWGTGTTVTVSS | ENNYKTTPPVLDSDGSFFLYSKLTVDKSRW | FGSGTKVTVL | VAPTECS (SEQ |
| | (SEQ ID NO: | QQGNVFSCSVMHEALHNHYTQKSLSLSPG | (SEQ ID NO: | ID NO: 415) |
| | 540) | (SEQ ID NO: 545) | 542) | |
| TA365 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGNSY | KATLVCLISDE |
| | VRQAPGKGLEWV | NTKVDKKVEPKSCDKTHTCPPCPAPELLGE | VSWYQQLPGTA | YPGAVTVAWKA |
| | SVISNSGGSTYY | ESVFLFPPKPKDTLMISRTPEVTCVVVDVS | PKLLIYLNSQR | DSSPVKAGVET |
| | TDSVKGRFTISR | HEDPEVKFNWYVDGVEVHNAKTKPREEQYN | PSGVPDRFSGS | TTPSKQSNNKY |
| | DNSKNTLYLQMN | STYRVVSVLIVLHQDWLNGKEYKCKVSNKA | KSGTSASLAIS | AASSYLSLTPE |
| | SLRAEDTAVYYC | LPAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 543) | 344) | |
| TA366 | EVQLLESGGGLV | ASTKGPSVFPLAPSSKSTSGGTAALGCLVK | QSVLTQPPSAS | GQPKAAPSVTL |
| | QPGGSLRLSCAA | DYFPEPVTVSWNSGALTSGVHTFPAVLQSS | GAPGQRVTISC | FPPSSEELQAN |
| | SGFTFSDYYMSW | GLYSLSSVVTVPSSSLGTQTYICNVNHKPS | SGSYSNIGNSY | KATLVCLISDE |
| | VRQAPGKGLEWV | NTKVDKKVEPKSCDKTHTCPPCPAPEFEGG | VSWYQQLPGTA | YPGAVTVAWKA |
| | SVISNSGGSTYY | PSVFLFPPKPKDTLMISRTPEVTCVVVDVS | PKLLIYLNSQR | DSSPVKAGVET |
| | TDSVKGRFTISR | DEDGEVKFNWYVDGVEVHNAKTKPREEQYN | PSGVPDRFSGS | TTPSKQSNNKY |
| | DNSKNTLYLQMN | STYRVVSVLTVLHQDWLNGKEYKCKVSNKA | KSGTSASLAIS | AASSYLSLTPE |
| | SLRAEDTAVYYC | LAAPIEKTISKAKGQPREPQVYTLPPSRDE | GLQSEDEADYY | QWKSHRSYSCQ |
| | TKDIGMTYFDYW | LTKNQVSLTCLVKGFYPSDIAVEWESNGQP | CAAWDDLNVWV | VTHEGSTVEKT |
| | GQGTLVTVSS | ENNYKTTPPVLDSDGSFFLYSKLTVDKSRW | FGGGTKLTVL | VAPTECS (SEQ |
| | (SEQ ID NO: | QQGNVFSCSVMHEALHNHYTQKSLSLSPG | (SEQ ID NO: | ID NO: 415) |
| | 530) | (SEQ ID NO: 545) | 344) | |

In some embodiments, the bispecific antibodies are comprised of four polypeptide chains comprising the following:
Chain 1: nt-VH1-CH1-CH2-CH3-Linker A-scFv [VL2-Linker Region B-VH2]-ct
Chain 2: nt-VH1-CH1-CH2-CH3-Linker A-scFv [VL2-Linker Region B-VH2]-ct
Chain 3: nt-VL1-CL-ct
Chain 4: nt-VL1-CL-ct.

In some embodiments, the bispecific antibodies are comprised of four polypeptide chains comprising the following:
Chain 1: nt-VH1-CH1-CH2-CH3-Linker A-scFv [VH2-Linker Region B-VL2]-ct
Chain 2: nt-VH1-CH1-CH2-CH3-Linker A-scFv [VH2-Linker Region B-VL2]-ct
Chain 3: nt-VL1-CL-ct
Chain 4: nt-VL1-CL-ct.

In some embodiments, the bispecific antibodies are comprised of four polypeptide chains that do not contain the Fc polypeptide, which can be illustrated as having the following formula:
Chain 1: nt-VH1-CH1-Linker A-scFv [VH2-Linker Region B-VL2]-ct
Chain 2: nt-VH1-CH1-Linker A-scFv [VH2-Linker Region B-VL2]-ct
Chain 3: nt-VL1-CL-ct
Chain 4: nt-VL1-CL-ct.

In some embodiments, the bispecific antibodies are comprised of four polypeptide chains that do not contain the Fc polypeptide, which can be illustrated as having the following formula:
Chain 1: nt-VH1-Linker A-scFv [VH2-Linker Region B-VL2]-ct
Chain 2: nt-VH1-Linker A-scFv [VH2-Linker Region B-VL2]-ct
Chain 3: nt-VL1-CL-ct
Chain 4: nt-VL1-CL-ct.

The Fc domain can be effectorless or can be an Fc domain that selectively binds to FcγRIIβ, such as those provided for herein.

In some embodiments, the bispecific antibodies are comprised of four polypeptide chains that do not contain the Fc polypeptide, which can be illustrated as having the following formula:
Chain 1: nt-VH1-CH1-Linker A-scFv [VL2-Linker Region B-VH2]-ct
Chain 2: nt-VH1-CH1-Linker A-scFv [VL2-Linker Region B-VH2]-ct
Chain 3: nt-VL1-CL-ct
Chain 4: nt-VL1-CL-ct.

In some embodiments, the bispecific antibodies are comprised of four polypeptide chains that do not contain the Fc polypeptide, which can be illustrated as having the following formula:
Chain 1: nt-VH1-Linker A-scFv [VL2-Linker Region B-VH2]-ct
Chain 2: nt-VH1-Linker A-scFv [VL2-Linker Region B-VH2]-ct
Chain 3: nt-VL1-CL-ct
Chain 4: nt-VL1-CL-ct.

In some embodiments, the bispecific antibodies are comprised of four polypeptide chains comprising the following:
Chain 1: nt-VH1-CH1-CH2-CH3-ct
Chain 2: nt-VH1-CH1-CH2-CH3-ct
Chain 3: nt-VL1-CL-ct
Chain 4: nt-VL1-CL-ct.

In some embodiments, the bispecific antibodies are comprised of four polypeptide chains comprising the following:
Chain 1: nt-VH1-CH1-CH2-CH3-Linker A-scFv [VL2-Linker Region B-VH2]-ct
Chain 2: nt-VH1-CH1-CH2-CH3-Linker A-scFv [VL2-Linker Region B-VH2]-ct
Chain 3: nt-VL1-Ck-ct
Chain 4: nt-VL1-Ck-ct.

In some embodiments, the bispecific antibodies are comprised of four polypeptide chains comprising the following:
Chain 1: nt-VH1-CH1-CH2-CH3-Linker A-scFv [VH2-Linker Region B-VL2]-ct
Chain 2: nt-VH1-CH1-CH2-CH3-Linker A-scFv [VH2-Linker Region B-VL2]-ct
Chain 3: nt-VL1-Ck-ct
Chain 4: nt-VL1-Ck-ct.

In some embodiments, the bispecific antibodies are comprised of four polypeptide chains that do not contain the Fc polypeptide, which can be illustrated as having the following formula:
Chain 1: nt-VH1-CH1-Linker A-scFv [VH2-Linker Region B-VL2]-ct
Chain 2: nt-VH1-CH1-Linker A-scFv [VH2-Linker Region B-VL2]-ct
Chain 3: nt-VL1-Ck-ct
Chain 4: nt-VL1-Ck-ct.

In some embodiments, the bispecific antibodies are comprised of four polypeptide chains that do not contain the Fc polypeptide, which can be illustrated as having the following formula:
Chain 1: nt-VH1—Linker A-scFv [VH2-Linker Region B-VL2]-ct
Chain 2: nt-VH1-Linker A-scFv [VH2-Linker Region B-VL2]-ct
Chain 3: nt-VL1-Ck-ct
Chain 4: nt-VL1-Ck-ct.

The Fc domain can be effectorless or can be an Fc domain that selectively binds to FcγRIIβ, such as those provided for herein.

In some embodiments, the bispecific antibodies are comprised of four polypeptide chains that do not contain the Fc polypeptide, which can be illustrated as having the following formula:
Chain 1: nt-VH1-CH1-Linker A-scFv [VL2-Linker Region B-VH2]-ct
Chain 2: nt-VH1-CH1-Linker A-scFv [VL2-Linker Region B-VH2]-ct
Chain 3: nt-VL1-Ck-ct
Chain 4: nt-VL1-Ck-ct.

In some embodiments, the bispecific antibodies are comprised of four polypeptide chains that do not contain the Fc polypeptide, which can be illustrated as having the following formula:
Chain 1: nt-VH1—Linker A-scFv [VL2-Linker Region B-VH2]-ct
Chain 2: nt-VH1-Linker A-scFv [VL2-Linker Region B-VH2]-ct
Chain 3: nt-VL1-Ck-ct
Chain 4: nt-VL1-Ck-ct.

In some embodiments, the bispecific antibodies are comprised of four polypeptide chains comprising the following:
Chain 1: nt-VH1-CH1-CH2-CH3-ct
Chain 2: nt-VH1-CH1-CH2-CH3-ct
Chain 3: nt-VL1-Ck-ct
Chain 4: nt-VL1-Ck-ct.

In some embodiments, the VH1 comprises an amino acid sequence of any VH selected from those in Table 9, Table 10 or Table 11. In some embodiments, the VH1 comprises an amino acid sequence of any one of SEQ ID NOs: 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 530, 538, and 540.

In some embodiments, the VL1 comprises an amino acid sequence of any VL selected from those in Table 9, Table 10 or Table 11. In some embodiments, the VL1 comprises an amino acid sequence of any one of SEQ ID NOs: 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 531, 532, 533, 534, 535, 536, 537, 539, 541, and 542.

In some embodiments, the CH1-CH2-CH3 comprises an amino acid sequence of any Fc provided herein. In some embodiments, the CH1-CH2-CH3 comprises an amino acid sequence of any Fc selected from those in Table 9, Table 10 or Table 11.

In some embodiments, the bispecific antibody comprises a VH1 comprising an amino acid sequence of any VH selected from those in Table 9, Table 10 or Table 11; a VL1 comprising an amino acid sequence of any VL selected from those in Table 9, Table 10 or Table 11; and a CH1-CH2-CH3 comprising an amino acid sequence of any Fc selected from those in Table 9, Table 10 or Table 11.

In some embodiments, the bispecific antibody comprises a VH1 comprising an amino acid sequence of any one of SEQ ID NOs: 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 530, 538, and 540; a VL1 comprising an amino acid sequence of any one of SEQ ID NOs: 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 531, 532, 533, 534, 535, 536, 537, 539, 541, and 542; and a CH1-CH2-CH3 comprising an amino acid sequence of any Fc selected from any one of SEQ ID NOs: 417, 418, 419, 420, 421, 422, 423, 424, 425, 426, 427, 428, 429, 430, 431, 432, 434, 435, 436, 437, 438, 439, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 543, 544, 545, 546, 683, 684, 685, 686, 687, 688, 689, and 690.

In some embodiments, the bispecific antibody comprises a VH1 comprising an amino acid sequence of SEQ ID NO: 136; a VL1 comprising an amino acid sequence of SEQ ID NO: 323; and a CH1-CH2-CH3 comprising an amino acid sequence of SEQ ID NO: 543. In some embodiments, the bispecific antibody comprises a VH1 comprising an amino acid sequence of SEQ ID NO: 136; a VL1 comprising an amino acid sequence of SEQ ID NO: 323; and a CH1-CH2-CH3 comprising an amino acid sequence of SEQ ID NO: 544. In some embodiments, the bispecific antibody comprises a VH1 comprising an amino acid sequence of SEQ ID NO: 136; a VL1 comprising an amino acid sequence of SEQ ID NO: 323; and a CH1-CH2-CH3 comprising an amino acid sequence of SEQ ID NO: 545. In some embodiments, the bispecific antibody comprises a VH1 comprising an amino acid sequence of SEQ ID NO: 156; a VL1 comprising an amino acid sequence of SEQ ID NO: 323; and a CH1-CH2-CH3 comprising an amino acid sequence of SEQ ID NO: 543. In some embodiments, the bispecific antibody comprises a VH1 comprising an amino acid sequence of SEQ ID NO: 156; a VL1 comprising an amino acid sequence of SEQ ID NO: 323; and a CH1-CH2-CH3 comprising an amino acid sequence of SEQ ID NO: 544. In some embodiments, the bispecific antibody comprises a VH1 comprising an amino acid sequence of SEQ ID NO: 156; a VL1 comprising an amino acid sequence of SEQ ID NO: 323; and a CH1-CH2-CH3 comprising an amino acid sequence of SEQ ID NO: 545. In some embodiments, the bispecific antibody comprises a VH1 comprising an amino acid sequence of SEQ ID NO: 187; a VL1 comprising an amino acid sequence of SEQ ID NO: 344; and a CH1-CH2-CH3 comprising an amino acid sequence of SEQ ID NO: 543. In some embodiments, the bispecific antibody comprises a VH1 comprising an amino acid sequence of SEQ ID NO: 187; a VL1 comprising an amino acid sequence of SEQ ID NO: 344; and a CH1-CH2-CH3 comprising an amino acid sequence of SEQ ID NO: 544. In some embodiments, the bispecific antibody comprises a VH1 comprising an amino acid sequence of SEQ ID NO: 187; a VL1 comprising an amino acid sequence of SEQ ID NO: 344; and a CH1-CH2-CH3 comprising an amino acid sequence of SEQ ID NO: 545. In some embodiments, the bispecific antibody comprises a VH1 comprising an amino acid sequence of SEQ ID NO: 290; a VL1 comprising an amino acid sequence of SEQ ID NO: 388; and a CH1-CH2-CH3 comprising an amino acid sequence of SEQ ID NO: 543. In some embodiments, the bispecific antibody comprises a VH1 comprising an amino acid sequence of SEQ ID NO: 290; a VL1 comprising an amino acid sequence of SEQ ID NO: 388; and a CH1-CH2-CH3 comprising an amino acid sequence of SEQ ID NO: 544. In some embodiments, the bispecific antibody comprises a VH1 comprising an amino acid sequence of SEQ ID NO: 290; a VL1 comprising an amino acid sequence of SEQ ID NO: 388; and a CH1-CH2-CH3 comprising an amino acid sequence of SEQ ID NO: 545. In some embodiments, the bispecific antibody comprises a VH1 comprising an amino acid sequence of SEQ ID NO: 290; a VL1 comprising an amino acid sequence of SEQ ID NO: 388; and a CH1-CH2-CH3 comprising an amino acid sequence of SEQ ID NO: 546. In some embodiments, the bispecific antibody comprises a VH1 comprising an amino acid sequence of SEQ ID NO: 136; a VL1 comprising an amino acid sequence of SEQ ID NO: 323; and a CH1-CH2-CH3 comprising an amino acid sequence of SEQ ID NO: 546. In some embodiments, the bispecific antibody comprises a VH1 comprising an amino acid sequence of SEQ ID NO: 156; a VL1 comprising an amino acid sequence of SEQ ID NO: 323; and a CH1-CH2-CH3 comprising an amino acid sequence of SEQ ID NO: 546. In some embodiments, the bispecific antibody comprises a VH1 comprising an amino acid sequence of SEQ ID NO: 187; a VL1 comprising an amino acid sequence of SEQ ID NO: 344; and a CH1-CH2-CH3 comprising an amino acid sequence of SEQ ID NO: 546. In some embodiments, the bispecific antibody comprises a VH1 comprising an amino acid sequence of SEQ ID NO: 530; a VL1 comprising an amino acid sequence of SEQ ID NO: 531; and a CH1-CH2-CH3 comprising an amino acid sequence of SEQ ID NO: 543. In some embodiments, the bispecific antibody comprises a VH1 comprising an amino acid sequence of SEQ ID NO: 530; a VL1 comprising an amino acid sequence of SEQ ID NO: 532; and a CH1-CH2-CH3 comprising an amino acid sequence of SEQ ID NO: 543. In some embodiments, the bispecific antibody comprises a VH1 comprising an amino acid sequence of SEQ ID NO: 530; a VL1 comprising an amino acid sequence of SEQ ID NO: 533; and a CH1-CH2-CH3 comprising an amino acid sequence of SEQ ID NO: 543. In some embodiments, the bispecific antibody comprises a VH1 comprising an amino acid sequence of SEQ ID NO: 530; a VL1 comprising an amino acid sequence of SEQ ID NO: 534; and a CH1-CH2-CH3 comprising an amino acid sequence of SEQ ID NO: 543. In some embodiments, the bispecific antibody comprises a VH1 comprising an amino acid sequence of SEQ ID NO: 530; a VL1 comprising an amino acid sequence of SEQ ID NO: 535; and a CH1-CH2-CH3 comprising an amino acid sequence of SEQ ID NO: 543. In some embodiments, the bispecific antibody comprises a VH1 comprising an amino acid sequence of SEQ ID NO: 530; a VL1 comprising an amino acid sequence of SEQ ID NO: 536; and a CH1-CH2-CH3 comprising an amino acid sequence of SEQ ID NO: 543. In some embodiments, the bispecific antibody comprises a VH1 comprising an amino acid sequence of SEQ ID NO: 530; a VL1 comprising an amino acid sequence of SEQ ID NO: 537; and a CH1-CH2-CH3 comprising an amino acid sequence of SEQ ID NO: 543. In some embodiments, the bispecific antibody comprises a VH1 comprising an amino acid sequence of SEQ ID NO: 530; a VL1 comprising an amino acid sequence of SEQ ID NO: 531; and a CH1-CH2-CH3 comprising an amino acid sequence of SEQ ID NO: 544. In some embodiments, the bispecific antibody comprises a VH1 comprising an amino acid sequence of SEQ ID NO: 530; a VL1 comprising an amino acid sequence of SEQ ID NO: 532; and a CH1-CH2-CH3 comprising an amino acid sequence of SEQ ID NO: 544. In some embodiments, the bispecific antibody comprises a VH1 comprising an amino acid sequence of SEQ ID NO: 530; a VL1 comprising an amino acid sequence of SEQ ID NO: 533; and a CH1-CH2-CH3 comprising an amino acid sequence of SEQ ID NO: 544. In some embodiments, the bispecific antibody comprises a VH1 comprising an amino acid sequence of SEQ ID NO: 530; a VL1 comprising an amino acid sequence of SEQ ID NO: 534; and a CH1-CH2-CH3 comprising an amino acid sequence of SEQ ID NO: 544. In some embodiments, the bispecific antibody comprises a VH1 comprising an amino acid sequence of SEQ ID NO: 530; a VL1 comprising an amino acid sequence of SEQ ID NO: 535; and a CH1-CH2-CH3 comprising an amino acid sequence of SEQ ID NO: 544. In some embodiments, the bispecific antibody comprises a VH1 comprising an amino acid sequence of SEQ ID NO: 530; a VL1 comprising an amino acid sequence of SEQ ID NO: 536; and a CH1-CH2-CH3 comprising an amino acid sequence of SEQ ID NO: 544. In some embodiments, the bispecific antibody comprises a VH1 comprising an amino acid sequence of SEQ ID NO: 530; a VL1 comprising an amino acid sequence of SEQ ID NO: 537; and a CH1-CH2-CH3 comprising an amino acid sequence of SEQ ID NO: 544. In some embodiments, the bispecific antibody comprises a VH1 comprising an amino acid sequence of SEQ ID NO: 530; a VL1 comprising an amino acid sequence of SEQ ID NO: 531; and a CH1-CH2-CH3 comprising an amino acid sequence of SEQ ID NO: 545. In some embodiments, the bispecific antibody comprises a VH1 comprising an amino acid sequence of SEQ ID NO: 530; a VL1 comprising an amino acid sequence of SEQ ID NO: 532; and a CH1-CH2-CH3 comprising an amino acid sequence of SEQ ID NO: 545. In some embodiments, the bispecific antibody comprises a VH1 comprising an amino acid sequence of SEQ ID NO: 530; a VL1 comprising an amino acid sequence of SEQ ID NO: 533; and a CH1-CH2-CH3 comprising an amino acid sequence of SEQ ID NO: 545. In some embodiments, the bispecific antibody comprises a VH1 comprising an amino acid sequence of SEQ ID NO: 530; a VL1 comprising an amino acid sequence of SEQ ID NO: 534; and a CH1-CH2-CH3 comprising an amino acid sequence of SEQ ID NO: 545. In some embodiments, the bispecific antibody comprises a VH1 comprising an amino acid sequence of SEQ ID NO: 530; a VL1 comprising an amino acid sequence of SEQ ID NO: 535; and a CH1-CH2-CH3 comprising an amino acid sequence of SEQ ID NO: 545. In some embodiments, the bispecific antibody comprises a VH1 comprising an amino acid sequence of SEQ ID NO: 530; a VL1 comprising an amino acid sequence of SEQ ID NO: 536; and a CH1-CH2-CH3 comprising an amino acid sequence of SEQ ID NO: 545. In some embodiments, the bispecific antibody comprises a VH1 comprising an amino acid sequence of SEQ ID NO: 530; a VL1 comprising an amino acid sequence of SEQ ID NO: 537; and a CH1-CH2-CH3 comprising an amino acid sequence of SEQ ID NO: 545. In some embodiments, the bispecific antibody comprises a VH1 comprising an amino acid sequence of SEQ ID NO: 538; a VL1 comprising an amino acid sequence of SEQ ID NO: 539; and a CH1-CH2-CH3 comprising an amino acid sequence of SEQ ID NO: 543. In some embodiments, the bispecific antibody comprises a VH1 comprising an amino acid sequence of SEQ ID NO: 538; a VL1 comprising an amino acid sequence of SEQ ID NO: 539; and a CH1-CH2-CH3 comprising an amino acid sequence of SEQ ID NO: 544. In some embodiments, the bispecific antibody comprises a VH1 comprising an amino acid sequence of SEQ ID NO: 538; a VL1 comprising an amino acid sequence of SEQ ID NO: 539; and a CH1-CH2-CH3 comprising an amino acid sequence of SEQ ID NO: 545. In some embodiments, the bispecific antibody comprises a VH1 comprising an amino acid sequence of SEQ ID NO: 540; a VL1 comprising an amino acid sequence of SEQ ID NO: 539; and a CH1-CH2-CH3 comprising an amino acid sequence of SEQ ID NO: 543. In some embodiments, the bispecific antibody comprises a VH1 comprising an amino acid sequence of SEQ ID NO: 540; a VL1 comprising an amino acid sequence of SEQ ID NO: 539; and a CH1-CH2-CH3 comprising an amino acid sequence of SEQ ID NO: 544. In some embodiments, the bispecific antibody comprises a VH1 comprising an amino acid sequence of SEQ ID NO: 540; a VL1 comprising an amino acid sequence of SEQ ID NO: 539; and a CH1-CH2-CH3 comprising an amino acid sequence of SEQ ID NO: 545. In some embodiments, the bispecific antibody comprises a VH1 comprising an amino acid sequence of SEQ ID NO: 540; a VL1 comprising an amino acid sequence of SEQ ID NO: 541; and a CH1-CH2-CH3 comprising an amino acid sequence of SEQ ID NO: 543. In some embodiments, the bispecific antibody comprises a VH1 comprising an amino acid sequence of SEQ ID NO: 540; a VL1 comprising an amino acid sequence of SEQ ID NO: 541; and a CH1-CH2-CH3 comprising an amino acid sequence of SEQ ID NO: 544. In some embodiments, the bispecific antibody comprises a VH1 comprising an amino acid sequence of SEQ ID NO: 540; a VL1 comprising an amino acid sequence of SEQ ID NO: 541; and a CH1-CH2-CH3 comprising an amino acid sequence of SEQ ID NO: 545. In some embodiments, the bispecific antibody comprises a VH1 comprising an amino acid sequence of SEQ ID NO: 538; a VL1 comprising an amino acid sequence of SEQ ID NO: 542; and a CH1-CH2-CH3 comprising an amino acid sequence of SEQ ID NO: 543. In some embodiments, the bispecific antibody comprises a VH1 comprising an amino acid sequence of SEQ ID NO: 538; a VL1 comprising an amino acid sequence of SEQ ID NO: 542; and a CH1-CH2-CH3 comprising an amino acid sequence of SEQ ID NO: 544. In some embodiments, the bispecific antibody comprises a VH1 comprising an amino acid sequence of SEQ ID NO: 538; a VL1 comprising an amino acid sequence of SEQ ID NO: 542; and a CH1-CH2-CH3 comprising an amino acid sequence of SEQ ID NO: 545. In some embodiments, the bispecific antibody comprises a VH1 comprising an amino acid sequence of SEQ ID NO: 540; a VL1 comprising an amino acid sequence of SEQ ID NO: 542; and a CH1-CH2-CH3 comprising an amino acid sequence of SEQ ID NO: 543. In some embodiments, the bispecific antibody comprises a VH1 comprising an amino acid sequence of SEQ ID NO: 540; a VL1 comprising an amino acid sequence of SEQ ID NO: 542; and a CH1-CH2-CH3 comprising an amino acid sequence of SEQ ID NO: 544. In some embodiments, the bispecific antibody comprises a VH1 comprising an amino acid sequence of SEQ ID NO: 540; a VL1 comprising an amino acid sequence of SEQ ID NO: 542; and a CH1-CH2-CH3 comprising an amino acid sequence of SEQ ID NO: 545. In some embodiments, the bispecific antibody comprises a VH1 comprising an amino acid sequence of SEQ ID NO: 530; a VL1 comprising an amino acid sequence of SEQ ID NO: 344; and a CH1-CH2-CH3 comprising an amino acid sequence of SEQ ID NO: 543. In some embodiments, the bispecific antibody comprises a VH1 comprising an amino acid sequence of SEQ ID NO: 530; a VL1 comprising an amino acid sequence of SEQ ID NO: 344; and a CH1-CH2-CH3 comprising an amino acid sequence of SEQ ID NO: 545.

In some embodiments, the bispecific antibody comprises a VH1 comprising an amino acid sequence of any VH selected from those in Table 9, Table 10 or Table 11; a VL1 comprising an amino acid sequence of any VL selected from those in Table 9, Table 10 or Table 11; a CH1-CH2-CH3 comprising an amino acid sequence of any Fc selected from those in Table 9, Table 10 or Table 11; and a Ck comprising an amino acid sequence of SEQ ID NO: 415.

In some embodiments, the bispecific antibody comprises a VH1 comprising an amino acid sequence of any one of SEQ ID NOs: 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 530, 538, and 540; a VL1 comprising an amino acid sequence of any one of SEQ ID NOs: 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 531, 532, 533, 534, 535, 536, 537, 539, 541, and 542; a CH1-CH2-CH3 comprising an amino acid sequence of any Fc selected from any one of SEQ ID NOs: 417, 418, 419, 420, 421, 422, 423, 424, 425, 426, 427, 428, 429, 430, 431, 432, 434, 435, 436, 437, 438, 439, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 543, 544, 545, 546, 683, 684, 685, 686, 687, 688, 689, and 690; and a Ck comprising an amino acid sequence of SEQ ID NO: 415.

In some embodiments, the bispecific antibody comprises a VH1 comprising an amino acid sequence of SEQ ID NO: 136; a VL1 comprising an amino acid sequence of SEQ ID NO: 323; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 136; a VL1 comprising an amino acid sequence of SEQ ID NO: 323; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 136; a VL1 comprising an amino acid sequence of SEQ ID NO: 323; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 156; a VL1 comprising an amino acid sequence of SEQ ID NO: 323; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 156; a VL1 comprising an amino acid sequence of SEQ ID NO: 323; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 156; a VL1 comprising an amino acid sequence of SEQ ID NO: 323; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 187; a VL1 comprising an amino acid sequence of SEQ ID NO: 344; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 187; a VL1 comprising an amino acid sequence of SEQ ID NO: 344; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 187; a VL1 comprising an amino acid sequence of SEQ ID NO: 344; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 290; a VL1 comprising an amino acid sequence of SEQ ID NO: 388; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 290; a VL1 comprising an amino acid sequence of SEQ ID NO: 388; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 290; a VL1 comprising an amino acid sequence of SEQ ID NO: 388; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 290; a VL1 comprising an amino acid sequence of SEQ ID NO: 388; a CH1-CH2-CH3 comprising an amino acid sequence of SEQ ID NO: 546; and a Ck comprising an amino acid sequence of SEQ ID NO: 415. In some embodiments, the bispecific antibody comprises a VH1 comprising an amino acid sequence of SEQ ID NO: 136; a VL1 comprising an amino acid sequence of SEQ ID NO: 323; a CH1-CH2-CH3 comprising an amino acid sequence of SEQ ID NO: 546; and a Ck comprising an amino acid sequence of SEQ ID NO: 415. In some embodiments, the bispecific antibody comprises a VH1 comprising an amino acid sequence of SEQ ID NO: 156; a VL1 comprising an amino acid sequence of SEQ ID NO: 323; a CH1-CH2-CH3 comprising an amino acid sequence of SEQ ID NO: 546; and a Ck comprising an amino acid sequence of SEQ ID NO: 415. In some embodiments, the bispecific antibody comprises a VH1 comprising an amino acid sequence of SEQ ID NO: 187; a VL1 comprising an amino acid sequence of SEQ ID NO: 344; a CH1-CH2-CH3 comprising an amino acid sequence of SEQ ID NO: 546; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 531; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 532; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 533; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 534; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 535; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 536; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 537; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 531; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 532; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 533; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 534; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 535; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 536; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 537; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 531; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 532; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 533; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 534; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 535; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 536; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 537; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 538; a VL1 comprising an amino acid sequence of SEQ ID NO: 539; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 538; a VL1 comprising an amino acid sequence of SEQ ID NO: 539; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 538; a VL1 comprising an amino acid sequence of SEQ ID NO: 539; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 540; a VL1 comprising an amino acid sequence of SEQ ID NO: 539; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 540; a VL1 comprising an amino acid sequence of SEQ ID NO: 539; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 540; a VL1 comprising an amino acid sequence of SEQ ID NO: 539; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 540; a VL1 comprising an amino acid sequence of SEQ ID NO: 541; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 540; a VL1 comprising an amino acid sequence of SEQ ID NO: 541; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 540; a VL1 comprising an amino acid sequence of SEQ ID NO: 541; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 538; a VL1 comprising an amino acid sequence of SEQ ID NO: 542; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 538; a VL1 comprising an amino acid sequence of SEQ ID NO: 542; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 538; a VL1 comprising an amino acid sequence of SEQ ID NO: 542; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 540; a VL1 comprising an amino acid sequence of SEQ ID NO: 542; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 540; a VL1 comprising an amino acid sequence of SEQ ID NO: 542; a CH1-CH2-CH3 comprising an amino acid sequence of SEQ ID NO: 544; and a Ck comprising an amino acid sequence of SEQ ID NO: 415. In some embodiments, the bispecific antibody comprises a VH1 comprising an amino acid sequence of SEQ ID NO: 540; a VL1 comprising an amino acid sequence of SEQ ID NO: 542; a CH1-CH2-CH3 comprising an amino acid sequence of SEQ ID NO: 545; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 344; a CH1-CH2-CH3 comprising an amino acid sequence of SEQ ID NO: 543; and a Ck comprising an amino acid sequence of SEQ ID NO: 415. In some embodiments, the bispecific antibody comprises a VH1 comprising an amino acid sequence of SEQ ID NO: 530; a VL1 comprising an amino acid sequence of SEQ ID NO: 344; a CH1-CH2-CH3 comprising an amino acid sequence of SEQ ID NO: 545; and a Ck comprising an amino acid sequence of SEQ ID NO: 415.

In some embodiments, chains 1 and 2 are identical to each other, and chains 3 and 4 are identical to each other. In some embodiments, chains 3 and 4 are identical and chains 1 and 2 are different from one another or are different from one another at the N or C terminus or both. In some embodiments, each of the chains have different sequences. In some embodiments, wherein chain 1 forms a homodimer with chain 2; and chain 3 and 4 associate with chain 1 and chain 2. That is, when each light chain associates with each heavy chain, VL1 associates with VH1 and CL associates with CH1 to form two functional Fab units. Without being bound to any particular theory, each scFv unit is intrinsically functional since VL2 and VH2 are covalently linked in tandem with a linker as provided herein (e.g. GGGGSG (SEQ ID NO: 13), GGGGS (SEQ ID NO: 1), GGGGSGGGGSGGGGSGGGGS (SEQ ID NO: 4), GGGGSGGGGSGGGGS (SEQ ID NO: 3) or GGGGSGGGGS (SEQ ID NO: 2)). The sequences of Linker A and Linker Region B, which are independent of one another can be the same or different and as otherwise described throughout the present application. Thus, in some embodiments, Linker A comprises GGGGS (SEQ ID NO: 1), GGGGSGGGGS (SEQ ID NO: 2), GGGGSGGGGSGGGGS (SEQ ID NO: 3), or GGGGSGGGGSGGGGSGGGGS (SEQ ID NO: 4). In some embodiments, Linker Region B comprises GGGGS (SEQ ID NO: 1), GGGGSGGGGS (SEQ ID NO: 2), GGGGSGGGGSGGGGS (SEQ ID NO: 3), or GGGGGGGGSGGGGSGGGGS (SEQ ID NO: 4). In some embodiments, Linker A comprises 1, 2, 3, 4, or 5 GGGGS repeats. In some embodiments, Linker Region B comprises 1, 2, 3, 4, or 5 GGGGS (SEQ ID NO: 1) repeats. For the avoidance of doubt, the sequences of Linker A and Linker Region B, which are used throughout this application, are independent of one another. Therefore, in some embodiments, Linker A and Linker Region B can be the same or different. In some embodiments, Linker A comprises GGGGS (SEQ ID NO: 1), GGGGSGGGGS (SEQ ID NO: 2), GGGGSGGGGSGGGGS (SEQ ID NO: 3), or GGGGGGGGSGGGGSGGGGS (SEQ ID NO: 4). In some embodiments, Linker Region B comprises GGGGS (SEQ ID NO: 1), GGGGSGGGGS (SEQ ID NO: 2), GGGGSGGGGSGGGGS (SEQ ID NO: 3), or GGGGSGGGGSGGGGSGGGGS (SEQ ID NO: 4). In some embodiments, the Linker A or Linker Region B comprises: GGGGS (SEQ ID NO: 1), (GGGGS)₃ (SEQ ID NO: 3), (GGGGS)ₙ (n=1, 2, 3, 4) (SEQ ID NO: 1-4), (Gly)₈ (SEQ ID NO: 5), (Gly)₆ (SEQ ID NO: 6). (EAAAK)₃ (SEQ ID NO: 7), (EAAK)ₙ (n=1-3) (SEQ ID NO: 8-10), A(EAAAK)₄ALEA(EAAAK)₄A (SEQ ID NO: 11), or AEAAAKEAAAKA (SEQ ID NO: 12).

The scFv may also be arranged in the NT-VH2-VL2-CT or NT-VL2-VH2-CT orientation. NT or nt stands for N-terminus and CT or ct stands for C-terminus of the protein. In some embodiments, the CH1, CH2, and CH3 are the domains from the IgG Fc polypeptide, and CL stands for Constant Light chain, which can be either kappa or lambda family light chains. The other definitions stand for the way they are normally used in the art. In some embodiments, the CH2 portions when present on the strands are different from one another. In some embodiments, the CH2 portions are the same.

In some embodiments, the compound comprises a light chain and a heavy chain. In some embodiments, the light and heavy chain begin at the N-terminus with the VH domain of a inhibitory receptor effector domain followed by the CH1 domain of a human IgG1, which is fused to a Fc polypeptide (e.g. CH2-CH3) of human IgG1. In some embodiments, at the c-terminus of the Fc polypeptide is fused to a linker as provided herein, such as but not limited to, GGGGS (SEQ ID NO; 1), GGGGSGGGGS (SEQ ID NO: 2) or GGGGSGGGGSGGGGS (SEQ ID NO: 3). The linker can then be fused to FcγRII binding effector domain. The polypeptides can dimerize because through the heavy chain dimerization, which results in a therapeutic compound having two effector moieties, such as two anti-PD-1 antibodies. However, where the antibodies bind to different molecules, they can form a heterodimer that bind to two different inhibitory receptors, such as, but not limited to those provided for herein, including PD-1 and LAG-3. In this orientation, the targeting moiety is an IgG format, there are two Fab arms that each recognize binding partner of the inhibitory receptor, for example, PD-1 being bound by the anti-PD-1 inhibitory receptor effector domain.

For the sake of clarity, in some embodiments, the VH1 and VL1 can form an antibody binding region that binds to FcγRII (i.e., is the FcγRII binding effector domain) and the scFv is the inhibitory receptor effector domain. In some embodiments, the VH1 and VL1 can form an antibody that is the inhibitory receptor effector domain and the scFv is the antibody that binds to FcγRII (i.e., is the FcγRII binding effector domain).

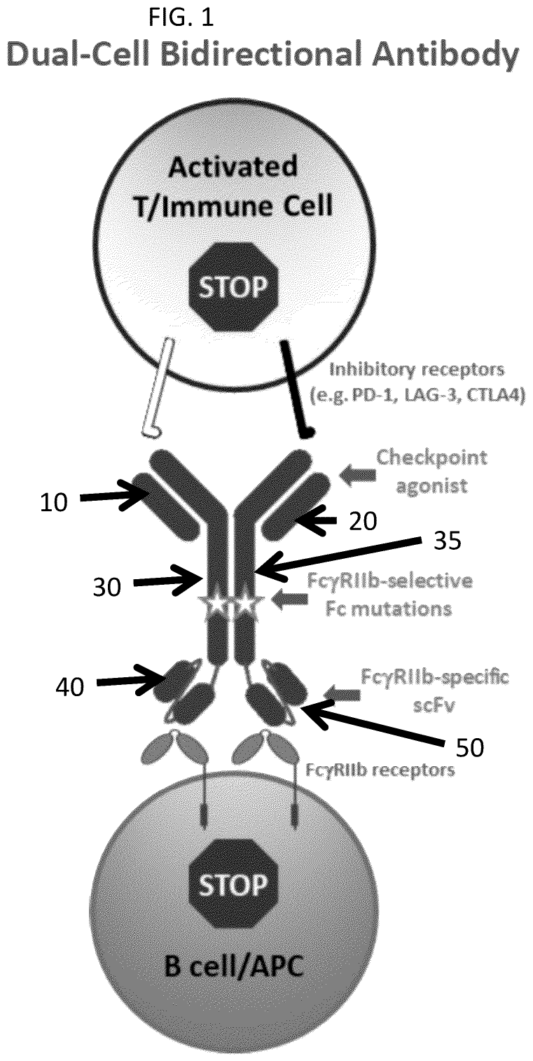

Another non-limiting example of a compound as provided for herein is illustrated in FIG. 1. Referencing FIG. 1, illustrates a dual targeted (bidirectional) antibody that can bind to two different cells at the same time or nearly simultaneously. Referencing FIG. 1 (10) illustrates a inhibitory receptor effector domain as an antibody (e.g. F′Ab2) that binds to an inhibitory receptor, such as PD-1, LAG3, or CTLA4. (20) illustrates another inhibitory receptor effector domain as an antibody that binds to an inhibitory receptor. The inhibitory receptor domains of (10) and (20) can bind to the same inhibitory receptor or different inhibitory receptors. Although illustrated as being a checkpoint agonist, the inhibitory receptor effector domains of (10) and (20) can be checkpoint antagonists as described herein.

Referencing FIGS. 1, (30) and (35) illustrate Fc domains that can comprise FcγRIIb selective mutations. Although illustrated as having FcγRIIb selective mutations the Fc polypeptide can also instead harbor FcγRIIα selective mutations. In such embodiments, if the Fc polypeptide comprises FcγRIIα selective mutations, the inhibitory receptor effector domain can comprise an inhibitory checkpoint antagonist.

Referencing FIG. 1, (40) and (50) illustrate FcγRII binding effector domains, which are shown as a scFv antibody. In some embodiments, (40) and (50) are the same, but they can have different structures, i.e. sequences. Additionally, (40) and (50) are illustrated as being FcγRIIb-specific scFv antibodies. However, (40) and (50) can also be being FcγRIIα-specific scFv antibodies.

Examples of formats for multispecific therapeutic compounds, e.g., bispecific antibody molecules are shown in the following non-limiting examples. Although illustrated with antibody molecules, they can be used as platforms for therapeutic molecules that include other non-antibody moieties as specific binding or effector moieties. In some embodiments, these non-limiting examples are based upon either a symmetrical or asymmetrical Fc formats.

For example, the figures illustrate non-limiting and varied symmetric homodimer approach. In some embodiments, the dimerization interface centers around human IgG CH2-CH3 domains of the Fc polypeptides selective for FcγRIIb, which dimerize via a contact interface spanning both CH2/CH2 and CH3/CH3. The resulting bispecific antibodies shown have a total valence comprised of four binding units with two identical binding units at the N-terminus on each side of the dimer and two identical units at the C-terminus on each side of the dimer. In each case the binding units at the N-terminus of the homodimer are different from those at the C-terminus of the homodimer. Using this type of bivalency for both an inhibitory T cell receptor at either terminus of the molecule and bivalency for an FcγRIIb receptor can be achieved at either end of the molecule.

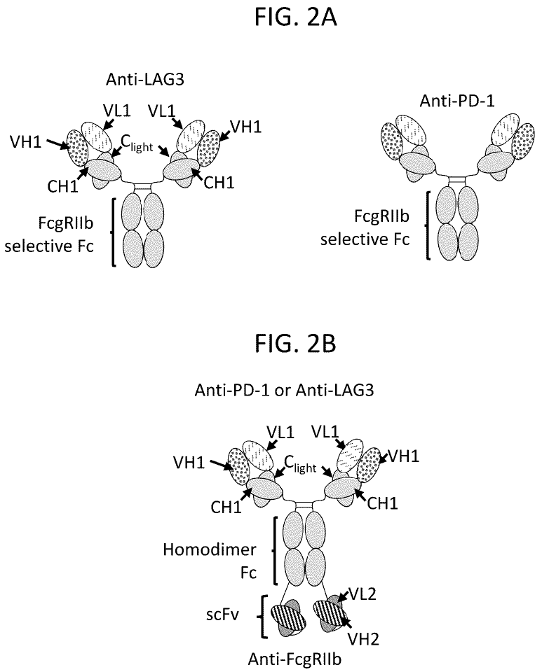

For example, in FIG. 2A, a non-limiting embodiment is illustrated. The N-terminus of the homodimer contains two identical Fab domains comprised of two identical light chains, which are separate polypeptides, interfaced with the N-terminal VH1-CH1 domains of each heavy chain via the VH/VL interaction and Clight interaction with CH1. The native disulfide bond between the Clight and CH1 is present providing a covalent anchor between the light and heavy chains. Further in FIG. 2B, at the C-terminus of the design shown in FIG. 2A are two identical scFv units where by (in this example) the C-terminus of the CH3 domain of the Fc, is followed by a flexible, hydrophilic linker typically comprised of (but not limited to) serine, glycine, alanine, and/or threonine residues, which is followed by the VH2 domain of each scFv unit, which is followed by a glycine/serine rich linker, followed by a VL2 domain. These tandem VH2 and VL2 domains associate to form a single chain fragment variable (scFv) appended at the C-terminus of the Fc. Two such units exist at the C-terminus of this molecule owing to the homodimeric nature centered at the Fc. The domain order of scFvs may be configured to be from N to C terminus either VH-Linker-VL or VL-Linker-VH.

A non-limiting example of a molecule that has different binding regions on the different ends is where, one end is an anti-PD-1 antibody, or an antigen-binding fragment thereof, and the other end is an anti-LAG3 antibody. This can be illustrated as shown, for example, in FIG. 3, which illustrates the molecules in different orientations.

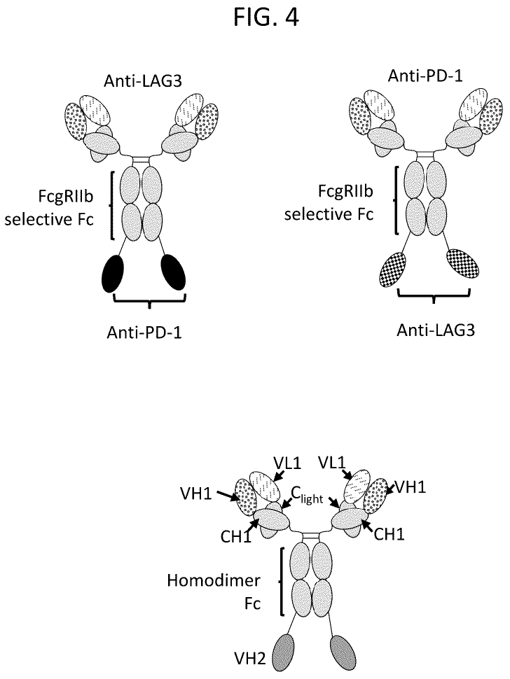

In another example, and as depicted in FIG. 4, the N-terminus of the homodimer contains two identical Fab domains comprised of two identical light chains, which are separate polypeptides, interfaced with the N-terminal VH1-CH1 domains of each heavy chain via the VH/VL interaction and Clight interaction with CH1. The native disulfide bond between the Clight and CH1 is present providing a covalent anchor between the light and heavy chains. At the C-terminus of this design are two identical VH2 units (though non-antibody moieties could also be substituted here or at any of the four terminal attachment/fusion points) where by (in this example) the C-terminus of the CH3 domain of the Fc, is followed by a flexible, hydrophilic linker typically comprised of (but not limited to) serine, glycine, alanine, and/or threonine residues, which is followed by a soluble independent VH2 domain. Two such units exist at the C-terminus of this molecule owing to the homodimeric nature centered at the Fc.

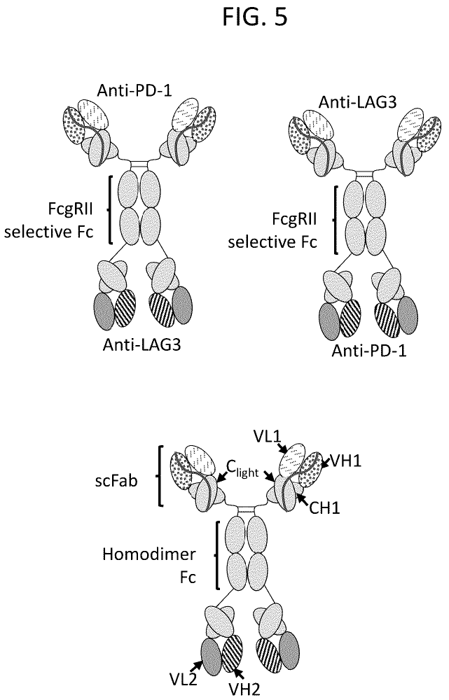

In another non-limiting example, as depicted in FIG. 5, the N-terminus of the homodimer contains two identical single chain Fab (scFab) domains comprised of two identical light chains, which, unlike FIG. 3 and FIG. 4, are physically conjoined with the heavy chain at the N-terminus via a Linker Region Between the C-terminus of Clight and the N-terminus of the VH1. The linker may be 36-80 amino acids in length and comprised of serine, glycine, alanine and threonine residues. The physically conjoined N-terminal light chains interface with the N-terminal VH1-CH1 domains of each heavy chain via the VH/VL interaction and Clight interaction with CH1. The native disulfide bond between the Clight and CH1 is present providing additional stability between the light and heavy chains. At the C-terminus of this design are two identical Fab units whereby (in this example) the C-terminus of the CH3 domain of the Fc, is followed by a flexible, hydrophilic linker typically comprised of (but not limited to) serine, glycine, alanine, and/or threonine residues, which is followed by a CH1 domain, followed by a VH2 domain at the C-terminus. The light chain that is designed to pair with the C-terminal CH1/VH2 domains is expressed as a separate polypeptide, unlike the N-terminal light chain which is conjoined to the N-terminal VH1/CH1 domains as described. The C-terminal light chains form an interface at between VH/VL and Clight with CH1. The native disulfide anchors this light chain to the heavy chain. Again, any of the antibody moieties at any of the four attachment/fusion points can be substituted with a non-antibody moiety, e.g., a effector binding/modulating moiety that does not comprise an antibody molecule.

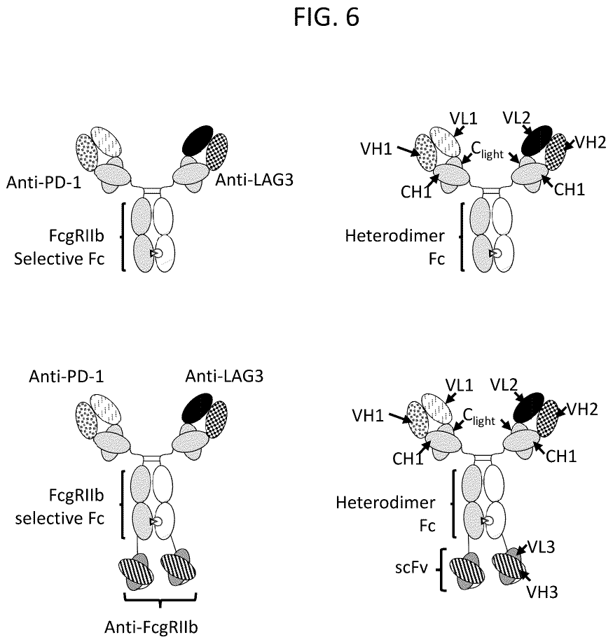

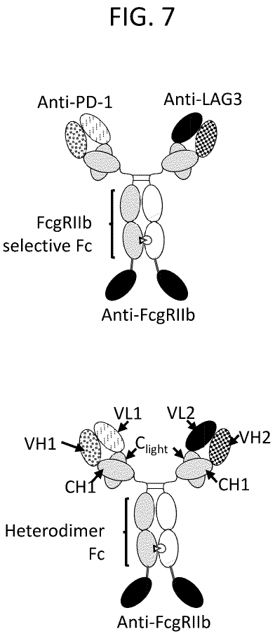

The bispecific antibodies can also be asymmetric as shown in the following non-limiting examples. FIG. 6 and FIG. 7 illustrate an asymmetric/heterodimer approach. In any of these formats, any of the antibody moieties at any of the four attachment/fusion points can be substituted with a non-antibody moiety, e.g., a effector binding/modulating moiety that does not comprise an antibody molecule. In some embodiments, the dimerization interface centers around the human IgG CH2-CH3 domains, which dimerize via a contact interface spanning both CH2/CH2 and CH3/CH3. However, in order to achieve heterodimerization instead of homodimerization of each heavy chain, mutations are introduced in each CH3 domain. The heterodimerizing mutations include T366W mutation (Kabat) in one CH3 domain and T366S, L368A, and Y407V (Kabat) mutations in the other CH3 domain. The heterodimerizing interface may be further stabilized with de novo disulfide bonds via mutation of native residues to cysteine residues such as S354 and Y349 on opposite sides of the CH3/CH3 interface. The resulting bispecific antibodies shown have a total valence comprised of four binding units. With this approach, the overall molecule can be designed to have bispecificity at just one terminus and monospecificity at the other terminus (trispecificity overall) or bispecificity at either terminus with an overall molecular specificity of 2 or 4. In the illustrative examples below, the C-terminus comprises two identical binding domains which could, for example, provide bivalent monospecificity for a tissue tethering target. At the N-terminus of all three of the illustrative examples, both binding domains comprise different recognition elements/paratopes and which could achieve recognition of two different epitopes on the same effector moiety target, or could recognize for examples a T cell inhibitory receptor and CD3. In some embodiments, the N-terminal binding moieties may be interchanged with other single polypeptide formats such as scFv, single chain Fab, tandem scFv, VH or VHH domain antibody configurations for example. Other types of recognition element may be used also, such as linear or cyclic peptides.

An example of an asymmetric molecule is depicted in FIG. 6. Referring to FIG. 6, the N-terminus of the molecule is comprised of a first light chain paired with a first heavy chain via VH/VL and Clight with CH1 interactions and a covalent tether comprised of the native heavy/light chain disulfide bond. On the opposite side of this heterodimeric molecule at the N-terminus is a second light chain paired with a second heavy chain via VH/VL and Clight with CH1 interactions and a covalent tether comprised of the native heavy/light chain disulfide bond. The physically conjoined N-terminal light chains interface with the N-terminal VH-CH1 domains of each heavy chain via the VH/VL interaction and Clight interaction with CH1. The native disulfide bond between the Clight and CH1 is present providing additional stability between the light and heavy chains. At the C-terminus of the molecule are two identical scFv units whereby, in this example, the C-terminus of the CH3 domain of the Fc, is followed by a flexible, hydrophilic linker typically comprised of (but not limited to) serine, glycine, alanine, and/or threonine residues, which is followed by the VH2 domain of each scFv unit, which is followed by a glycine/serine rich linker, followed by a VL2 domain. These tandem VH2 and VL2 domains associate to form a single chain fragment variable (scFv) appended at the C-terminus of the Fc. Two such units exist at the C-terminus of this molecule owing to the homodimeric nature centered at the Fc. The domain order of scFvs may be configured to be from N to C terminus either VH-Linker-VL or VL-Linker-VH.

Another example of an asymmetric molecule is depicted in FIG. 7. Referring to FIG. 7, the N-terminus of the molecule is comprised of a first light chain paired with a first heavy chain via VH/VL and Clight with CH1 interactions and a covalent tether comprised of the native heavy/light chain disulfide bond. On the opposite side of this heterodimeric molecule at the N-terminus is a second light chain paired with a second heavy chain via VH/VL and Clight with CH1 interactions and a covalent tether comprised of the native heavy/light chain disulfide bond. The physically conjoined N-terminal light chains interface with the N-terminal VH-CH1 domains of each heavy chain via the VH/VL interaction and Clight interaction with CH1. The native disulfide bond between the Clight and CH1 is present providing additional stability between the light and heavy chains. At the C-terminus of the molecule are two identical soluble VH germline based VH domains, which are identical on each of the two heavy chains. The heavy chain heterodimerizes via the previously described knobs into holes mutations present at the CH3 interface of the Fc module.

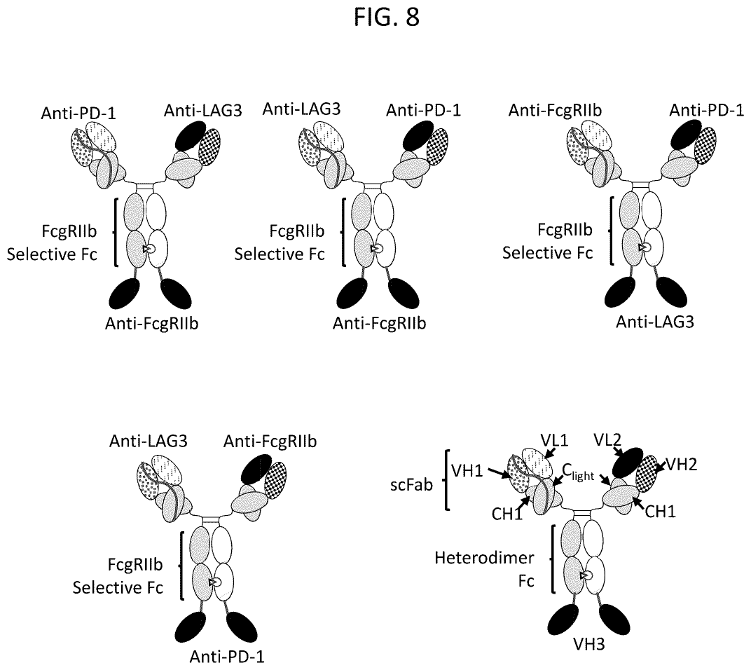

Referring to FIG. 8, the N-terminus of the molecule is comprised of a first light chain paired with a first heavy chain via VH/VL and Clight with CH1 interactions, and a covalent tether comprising the native heavy/light chain disulfide bond. On the opposite side of this heterodimeric molecule at the N-terminus is a second light chain and a second heavy chain which are physically conjoined via a Linker Region Between the C-terminus of Clight and the N-terminus of the VH1. The linker may be 36-80 amino acids in length and comprised of serine, glycine, alanine and threonine residues. The physically conjoined N-terminal light chains interface with the N-terminal VH1-CH1 domains of each heavy chain via the VH/VL interaction and Clight interaction with CH1. The native disulfide bond between the Clight and CH1 is present providing additional stability between the light and heavy chains. At the C-terminus of this molecule are two identical soluble VH3 germline family VH domains joined via an N-terminal glycine/serine/alanine/threonine based linker to the C-terminus of the CH3 domain of both heavy chain 1 and heavy chain 2 of the Fc dimer.

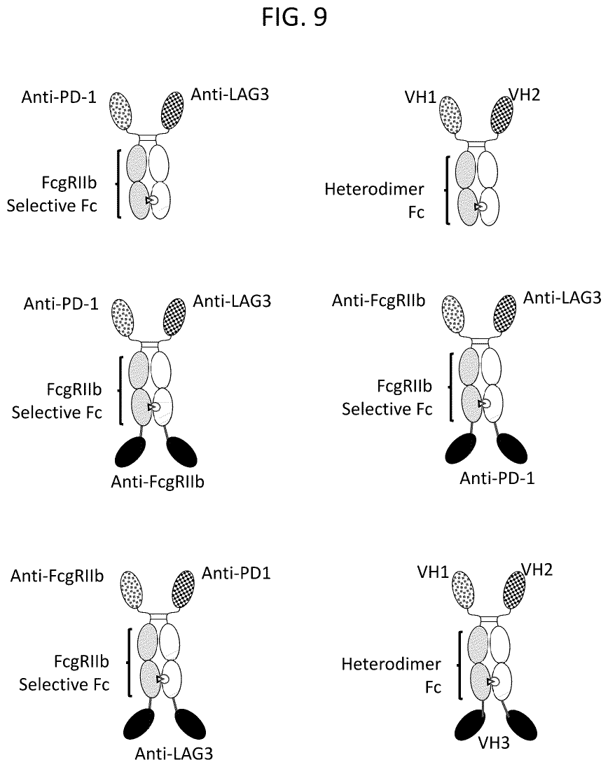

In some embodiments, an asymmetric molecule can be as illustrated as depicted in FIG. 9. For example, the N-terminus of the molecule is comprised of two different VH germlined based soluble VH domains linked to the human IgG1 hinge region via a glycine/serine/alanine/threonine based linker. The VH domain connected to the first heavy chain is different than the VH domain connected to the second heavy chain. At the C-terminus of each heavy chain is an additional soluble VH germline based VH domain, which is identical on each of the two heavy chains. The heavy chain heterodimerizes via the previously described knobs into holes mutations present at the CH3 interface of the Fc module.

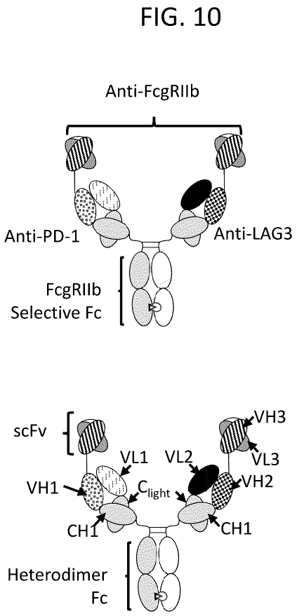

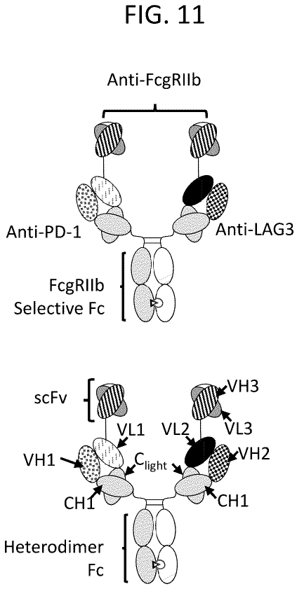

Other embodiments of trispecific molecules are illustrated in FIGS. 10 and 11. As illustrated in FIG. 10, the N-terminus of the molecule is comprised of a first light chain paired with a first heavy chain via VH/VL and Clight with CH1 interactions and a covalent tether comprised of the native heavy/light chain disulfide bond. On the opposite side of this heterodimeric molecule at the N-terminus is a second light chain paired with a second heavy chain via VH/VL and Clight with CH1 interactions and a covalent tether comprised of the native heavy/light chain disulfide bond. The physically conjoined N-terminal light chains interface with the N-terminal VH-CH1 domains of each heavy chain via the VH/VL interaction and Clight interaction with CH1. The native disulfide bond between the Clight and CH1 is present providing additional stability between the light and heavy chains. Further at the N-terminus of the molecule are two identical scFv units whereby, in this example, the N-terminus of the VH1 or VH2 domain of either Fab moiety, is followed by a flexible, hydrophilic linker typically comprised of (but not limited to) serine, glycine, alanine, and/or threonine residues, which is followed by the VH3 domain of each scFv unit, which is followed by a glycine/serine rich linker, followed by a VL3 domain. These tandem VH3 and VL3 domains associate to form a single chain fragment variable (scFv) domains appended at the N-terminus of the Fab molecule. As illustrated in FIG. 11, the N-terminus of the molecule is comprised of a first light chain paired with a first heavy chain via VH/VL and Clight with CH1 interactions and a covalent tether comprised of the native heavy/light chain disulfide bond. On the opposite side of this heterodimeric molecule at the N-terminus is a second light chain paired with a second heavy chain via VH/VL and Clight with CH1 interactions and a covalent tether comprised of the native heavy/light chain disulfide bond. The physically conjoined N-terminal light chains interface with the N-terminal VH-CH1 domains of each heavy chain via the VH/VL interaction and Clight interaction with CH1. The native disulfide bond between the Clight and CH1 is present providing additional stability between the light and heavy chains. Further at the N-terminus of the molecule are two identical scFv units whereby, in this example, the N-terminus of the VL1 or VL2 domain of either Fab moiety, is followed by a flexible, hydrophilic linker typically comprised of (but not limited to) serine, glycine, alanine, and/or threonine residues, which is followed by the VH3 domain of each scFv unit, which is followed by a glycine/serine rich linker, followed by a VL3 domain. These tandem VH3 and VL3 domains associate to form a single chain fragment variable (scFv) domains appended at the N-terminus of the Fab molecule.

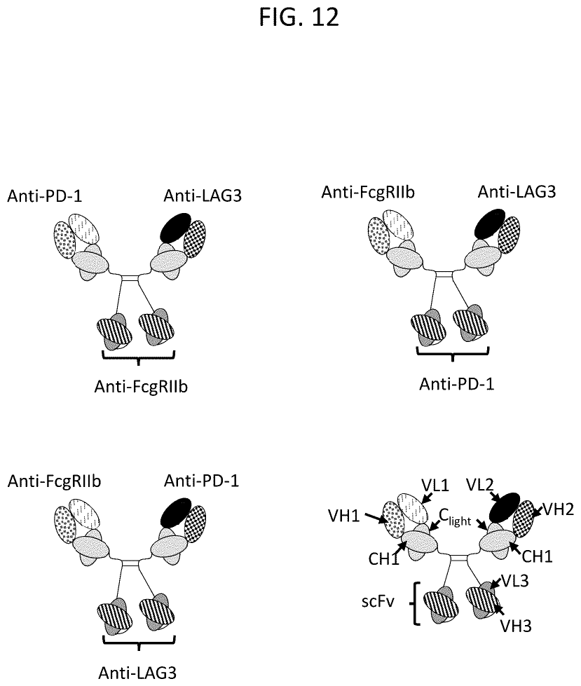

FIG. 12 illustrates another embodiment. FIG. 12 is a F (ab′) 2 scFv fusion. This consists of two Fab components having different specificity, joined via two disulfide bonds in the native human IgG hinge region C-terminal of the human IgG CH1 domain. The human IgG CH2 and CH3 domains are absent. At the C-terminus of heavy chains 1 and 2 are two identical scFv fragments linked via a gly/ser/ala/thr rich linker to the C-terminus of the human IgG hinge region. In the configuration shown, the VH is N-terminal in each scFv unit and linked via a 12-15 amino acid gly/ser rich linker to the N-terminus of a VL domain. An alternative configuration would be N-terminus-VL-Linker-VH-C-terminus. In this design, the construct is trispecific. Again, in this format, any of the antibody moieties at any of the four attachment/fusion points can be substituted with a non-antibody moiety, e.g., a effector binding/modulating moiety that does not comprise an antibody molecule.

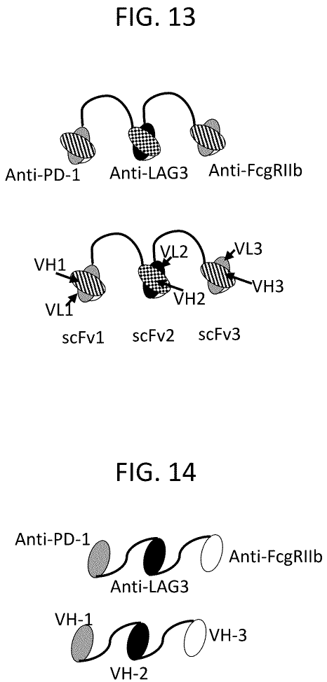

FIG. 13 represents a tandem scFv format consisting of a first N-terminal VL domain linked at its C-terminus to the N-terminus of a first VH domain with a 12-15 amino acid gly/ser rich linker, followed at the first VH C-terminus by a 25-30 amino acid gly/ser/ala/thr based linker to the N-terminus of a second VL domain. The second VL domain is linked at the C-terminus to the N-terminus of a second VH domain by a 12-15 amino acid gly/ser linker, followed at the second VH C-terminus by a 25-30 amino acid gly/ser/ala/thr based linker to the N-terminus of a third VL domain. The third VL domain is linked at the C-terminus to the N-terminus of a third VH domain by a 12-15 amino acid gly/ser linker. Each scFv recognizes a different target antigen such as a co-stimulatory T cell molecule and a FcγRIIb receptor. In this format, any of the antibody moieties can be substituted with a non-antibody moiety, e.g., a effector binding/modulating moiety that does not comprise an antibody molecule.

FIG. 14 illustrates another example. In this example, the molecule is comprised of three VH germline based soluble VH domains. Each of the three domains has different specificity. For example, the first domain from the N-terminus may have specificity for an inhibitory receptor, the second domain from the N-terminus may have specificity for another inhibitory receptor, and the third domain from the N-terminus may have specificity for a tissue antigen and the fourth domain from the N-terminus may have specificity for FcγRIIb. Two glycine, serine, alanine and/or threonine rich linkers exists between domains 1 and 2, and domains 2 and 3. This format may be configured with up to trispecificity, but monovalent in each case, or to have bispecificity with bivalency in each case. The order of domains can be changed. Again, in this format, any of the antibody moieties can be substituted with a non-antibody moiety, e.g., a effector binding/modulating moiety that does not comprise an antibody molecule.

In some embodiments, when the inhibitory receptor effector domain is a checkpoint agonist, the Fc polypeptide comprises mutations that are FcγRIIb selective mutations and the FcγRII binding effector domains is a FcγRIIb-specific scFv antibody.

In some embodiments, when the inhibitory receptor effector domain is a checkpoint antagonist, the Fc polypeptide comprises mutations that are FcγRIIα selective mutations and the FcγRII binding effector domains is a FcγRIIα-specific scFv antibody.

### Methods of Use

The compounds provided for herein can be used to treat auto-immune diseases. Thus, in some embodiments, embodiments are provided for methods of treating an autoimmune disease or disorder in a subject. In some embodiments, the methods comprise administering to the subject a compound as provided for herein. In some embodiments, the subject has or is at risk of having an autoimmune disorder. In some embodiments, the autoimmune disorder is Type 1 Diabetes, Multiple Sclerosis, Cardiomyositis, vitiligo, alopecia, inflammatory bowel disease (IBD, e.g. Crohn's disease or ulcerative colitis), Sjogren's syndrome, focal segmented glomerular sclerosis (FSGS), scleroderma/systemic sclerosis (SSc) or rheumatoid arthritis. In some embodiments, the treatment minimizes rejection of, minimizes immune effector cell mediated damage to, prolongs the survival of subject tissue undergoing, or a risk for, autoimmune attack, such as from a transplant.

Other examples of autoimmune disorders and diseases that can be treated with the compounds described herein include, but are not limited to, myocarditis, postmyocardial infarction syndrome, postpericardiotomy syndrome, subacute bacterial endocarditis, anti-glomerular basement membrane nephritis, interstitial cystitis, lupus nephritis, membranous glomerulonephropathy, chronic kidney disease (CKD), autoimmune hepatitis, primary biliary cirrhosis, primary sclerosing cholangitis, antisynthetase syndrome, alopecia areata, autoimmune angioedema, autoimmune progesterone dermatitis, overlap connective tissues disease syndromes, polymyalgia rheumatic, autoimmune urticaria, bullous pemphigoid, cicatricial pemphigoid, dermatitis herpetiformis, discoid lupus erythematosus, epidermolysis bullosa acquisita, erythema nodosum, anti-neutrophil cytoplasmic antibody associated vasculitis, Henoch-Schonlein purpura, Cogan's syndrome, Buerger's disease, Susan's disease, immune complex vasculitis, primary angiitis of the CNS, gestational pemphigoid, hidradenitis suppurativa, lichen planus, lichen sclerosus, linear iga disease (lad), morphea, pemphigus vulgaris, pityriasis lichenoides ct varioliformis acuta, mucha-habermann disease, psoriasis, systemic scleroderma, vitiligo, Addison's disease, autoimmune polyendocrine syndrome (APS) type 1, autoimmune polyendocrine syndrome (APS) type 2, juvenile idiopathic arthritis, juvenile dermatomyositis, autoimmune brain disease, autoimmune polyendocrine syndrome (APS) type 3, autoimmune pancreatitis (AIP), diabetes mellitus type 1, autoimmune thyroiditis, Ord's thyroiditis, Graves' disease, autoimmune oophoritis, endometriosis, autoimmune orchitis, Sjögren's syndrome, autoimmune enteropathy, Coeliac disease, Crohn's disease, microscopic colitis, ulcerative colitis, thrombocytopenia, adiposis, dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, lyme disease (chronic), mixed connective tissue disease (MCTD), palindromic rheumatism, Parry Romberg syndrome, Parsonage-Turner syndrome, psoriatic arthritis, IBD-associated arthritis, reactive arthritis, relapsing polychondritis, retroperitoneal fibrosis, rheumatic fever, rheumatoid arthritis, autoimmune complications of immune checkpoint inhibitors (IRAEs), sarcoidosis, neurosarcoidosis, Schnitzler syndrome, systemic lupus erythematosus (SLE), undifferentiated connective tissue disease (UCTD), dermatomyositis, IgG4 related disease, fibromyalgia, antiphospholipid syndrome, inclusion body myositis, myositis, myasthenia gravis, neuromyotonia, parancoplastic cerebellar degeneration, polymyositis, acute disseminated encephalomyelitis (ADEM), adult onset Still's disease, acute motor axonal neuropathy, anti-N-Methyl-D-Aspartate (anti-NMDA) receptor encephalitis, warm antibody hemolytic anemia (wAIHA), immune thrombocytopenia, immune thrombotic thrombocytopenia, thrombotic thrombocytopenia, pernicious anemia, aplastic anemia, Evan's syndrome, autoimmune neutropenia, acquired von Willibrand syndrome, recurring fetal loss, Rh mismatch, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, primary biliary sclerosis, glomerulonephritis, glomerular basement membrane disease, multiple sclerosis, Oshtoran syndrome, pediatric autoimmune neuropsychiatric disorder associated with Streptococcus (PANDAS), progressive inflammatory neuropathy, cutaneous lupus erythematosus, restless leg syndrome, pemphigus foliaceus including fogo selvage, transplantation, antibody-mediated rejection, alloantibody hypersensitization, xenoantibody mediated rejection, solid organ rejection, graft vs host disease acute and chronic, stiff person syndrome, Sydenham chorea, transverse myelitis, autoimmune retinopathy, autoimmune uveitis, uveitis, Cogan syndrome, Graves ophthalmopathy, amyotrophic lateral sclerosis (ALS), Parkinson's disease, autoimmune encephalitis, CNS vasculitis, chronic idiopathic demyelinating polyneuropathy (CIDP), keratitis, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, rheumatic heart disease, chronic rhinosinusitis with nasal polyps, allergic bronchoplmonary mycosis, hypersensitivity pneumonitis, rheumatoid arthritis-associated interstitial lung disease (RA-ILD), nonspecific interstitial pneumonia, allergic asthma, infectious disease/vaccination, antibody dependent enhancement (as with dengue virus infection), chronic meningitis, anti-myelin oligodendrocyte glycoprotein (MOG) disease, activated-DLBCL, anti-drug antibody, anti-gene therapy vector antibody (anti-AAV antibody), antibody to therapeutic biologic agents (cytokines, monoclonal antibodies, enzymes, coagulation factors), autoimmune inner ear disease (AIED), Ménière's disease, Behcet's disease, eosinophilic granulomatosis with polyangiitis (EGPA), giant cell arteritis, polyglandular autoimmune endocrine syndromes, granulmatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumaticia, vasculitis, primary immune deficiency, and the like.

Other examples of potential autoimmune disorders and diseases, as well as autoimmune comorbidities that can be treated with the compounds described herein include, but are not limited to, chronic fatigue syndrome, complex regional pain syndrome, eosinophilic esophagitis, gastritis, interstitial lung disease, POEMS syndrome, Raynaud's phenomenon, primary immunodeficiency, pyoderma gangrenosum, agammaglobulinemia, anyloidosis, anyotrophic lateral sclerosis, anti-tubular basement membrane nephritis, atopic allergy, atopic dermatitis, autoimmune peripheral neuropathy, Blau syndrome, Castleman's disease, Chagas disease, chronic obstructive pulmonary disease, chronic recurrent multifocal osteomyelitis, complement component 2 deficiency, contact dermatitis, Cushing's syndrome, cutaneous leukocytoclastic angiitis, Dego′ disease, eczema, eosinophilic gastroenteritis, eosinophilic pneumonia, erythroblastosis fetalsis, fibrodysplasia ossificans progressive, gastrointestinal pemphigoid, hypogammaglobulinemia, idiopathic giant-cell myocarditis, idiopathic pulmonary fibrosis, IgA nephropathy, immunoregulatory lipoproteins, IPEX syndrome, ligenous conjunctivitis, Majeed syndrome, narcolepsy, Rasmussen's encephalitis, schizophrenia, serum sickness, spondyloathropathy, Sweet's syndrome, Takayasu's arteritis, and the like.

In some embodiments, the autoimmune disorder does not comprise pemphigus vulgaris, pemphigus. In some embodiments, the autoimmune disorder does not comprise pemphigus foliaceus. In some embodiments, the autoimmune disorder does not comprise bullous pemphigoid. In some embodiments, the autoimmune disorder does not comprise Goodpasture's Disease. In some embodiments, the autoimmune disorder does not comprise psoriasis. In some embodiments, the autoimmune disorder does not comprise a skin disorder. In some embodiments, the disorder does not comprise a neoplastic disorder, e.g., cancer.

In some embodiments, the condition to be treated is a neoplastic disorder, such as a cancer. In contrast, to the molecule that is used to treat an autoimmune disorder the molecule is used to antagonize the inhibitor receptor to which the inhibitory receptor effector domain binds to. Additionally, the Fc polypeptide comprises mutations that are not inhibitory, such that they can be used to extend the half-life of the molecule. In some embodiments, the FcγRII binding effector domain binds preferentially to the FcγRIIb binding effector domain.

In some embodiments, the cancer is a solid or liquid tumor. In some embodiments, the liquid or solid tumor include, but are not limited to, hematopoietic cancer, lymphoid cancer, skin cancer, head and neck cancer, genitourinary cancer, blood cancer, lung cancer, breast cancer, brain cancer, esophageal cancer, colorectal cancer, pancreatic cancer, and any combination thereof.

In some embodiments, the antibodies, the molecules, the polypeptides provided for herein are used in a method of modulating two types of cells, the method comprising contacting the two types of cells with the antibody, the molecule, the polypeptide, or a pharmaceutical composition thereof. In some embodiments, one cell is a T-cell, NK Cell, Dendritic cell, and the like, and the second cell is a B-Cell, an antigen presenting cell (APC), or a myeloid cell.

In some embodiments, the antibodies, the molecules, the polypeptides provided for herein are used in a method of inhibiting an activated immune cell (e.g. T-cell) and the activity of a B-Cell, an antigen presenting cell (APC), or a myeloid cell, the method comprising contacting the activated immune cell and the B Cell or antigen presenting cell with the antibody, the molecule, the polypeptide, or a pharmaceutical composition thereof.

In some embodiments, the antibodies, the molecules, the polypeptides provided for herein are used in a method of activating or enhancing an activated immune cell (e.g. T-cell) and the activity of B-Cell, an antigen presenting cell (APC), or a myeloid cell, the method comprising contacting the activated immune cell and the B Cell or antigen presenting cell with the antibody, the molecule, the polypeptide, or a pharmaceutical composition thereof.

In some embodiments, the antibodies, the molecules, the polypeptides provided for herein are used in a method of inhibiting B cell activation, the method comprising administering the antibody, the molecule, the polypeptide, or a pharmaceutical composition thereof. In some embodiments, the antibody, the molecule, the polypeptide, or a pharmaceutical composition thereof selectively binds to FcγRIIβ to inhibit the activation of the B cell.

In some embodiments, the molecules (e.g. antibodies) provided for herein can be used to increase the number of Tregs (T-regulatory cells) in a subject or in vivo. In some embodiments, the molecules increase the percentage of CD4+ T cells that are FoxP3 positive, which can be referred to as FOXP3+ Tregs. In some embodiments, the percentage increase is at least 10%. In some embodiments, the percentage increase is at least 20%. In some embodiments, the percentage increase is at least 30%. In some embodiments, the percentage increase is at least 40%. In some embodiments, the percentage increase is at least 50%. In some embodiments, the percentage increase is at least 60%. In some embodiments, the percentage increase is at least 70%. In some embodiments, the percentage increase is at least 80%. In some embodiments, the percentage increase is at least 90%. In some embodiments, the percentage increase is at least 100%. In some embodiments, the percentage increase is at least 125%. In some embodiments, the percentage increase is at least 150%. In some embodiments, the percentage increase is at least 200%. In some embodiments, the percentage increase of FOXP3+ Tregs is determined by comparing an average of a population of patients that are untreated with an average of a population of patients that are treated with the molecule. In some embodiments, the percentage increase is determined by comparing a subject's FOXP3+ Tregs (FOXP3+, CD4+ T cells) before and after administration of the molecule. As provided for herein, the molecule can be administered by various types of administration, but in some embodiments, the number of FOXP3+ Tregs is measured after intravenous or subcutaneous administration of the molecule to determine the percentage increase.

In some embodiments, the molecules (e.g. antibodies) provided for herein can be used to increase the activation of Tregs in a in a subject or in vivo. In some embodiments, the Tregs are activated at least 10%. In some embodiments, the Tregs are activated at least 20%. In some embodiments, the Tregs are activated at least 30%. In some embodiments, the Tregs are activated at least 40%. In some embodiments, the Tregs are activated at least 50%. In some embodiments, the Tregs are activated at least 60%. In some embodiments, the Tregs are activated at least 70%. In some embodiments, the Tregs are activated at least 80%. In some embodiments, the Tregs are activated at least 90%. In some embodiments, the Tregs are activated at least 100%. In some embodiments, the Tregs are activated at least 90%. In some embodiments, the Tregs are activated at least 100%. In some embodiments, the Treg activation is determined by measuring the percentage of Treg cells (FOXP3+, CD4+ T cells) that are CD69+ before and after the molecule is administered to a subject or population of subjects. Without being bound to any particular theory, CD69+ expression on Tregs correlates with a more suppressive Treg population and/or a more activated population of Tregs. Thus, in some embodiments, the molecules can be used to increase the number of CD69+ Tregs, which can be characterized as CD69+, FOX3P+, CD4+ T cells. In some embodiments, the increase of CD69+ Tregs is increased by at least 10%. In some embodiments, the increase of CD69+ Tregs is increased by at least 20%. In some embodiments, the increase of CD69+ Tregs is increased by at least 30%. In some embodiments, the increase of CD69+ Tregs is increased by at least 40%. In some embodiments, the increase of CD69+ Tregs is increased by at least 50%. In some embodiments, the increase of CD69+ Tregs is increased by at least 60%. In some embodiments, the increase of CD69+ Tregs is increased by at least 70%. In some embodiments, the increase of CD69+ Tregs is increased by at least 80%. In some embodiments, the increase of CD69+ Tregs is increased by at least 90%. In some embodiments, the increase of CD69+ Tregs is increased by at least 100%. In some embodiments, the percent increase in CD69+ Tregs, the increase in activation, or percent increase in activation is determined on a population of patients that have been treated or untreated with the molecule. In some embodiments, the percent increase in CD69+ Tregs, the increase in activation, or percent increase in activation is determined in a single subject before and after administration of the molecule. As provided for herein, the molecule can be administered by various types of administration, but in some embodiments, the increase in activation (increase in CD69+ Tregs) is measured after intravenous or subcutaneous administration of the molecule to determine the increase or percentage increase.

In some embodiments, the molecules (e.g. antibodies) provided for herein can be used to increase TIGIT expression on Tregs or expand the TIGIT+ Treg population. Without being bound to any particular theory, TIGIT expression on Tregs correlates with a more suppressive Treg population. In some embodiments, the increase of TIGIT expression on Tregs (FOXP3+, CD4+ T Cells) or the expansion of TIGIT+ Treg population is increased by at least 10%. In some embodiments, the increase of TIGIT expression on Tregs (FOXP3+, CD4+ T Cells) or the expansion of TIGIT+ Treg population is increased by at least 20%. In some embodiments, the increase of TIGIT expression on Tregs (FOXP3+, CD4+ T Cells) or the expansion of TIGIT+ Treg population is increased by at least 30%. In some embodiments, the increase of TIGIT expression on Tregs (FOXP3+, CD4+ T Cells) or the expansion of TIGIT+ Treg population is increased by at least 40%. In some embodiments, the increase of TIGIT expression on Tregs (FOXP3+, CD4+ T Cells) or the expansion of TIGIT+ Treg population is increased by at least 50%. In some embodiments, the increase of TIGIT expression on Tregs (FOXP3+, CD4+ T Cells) or the expansion of TIGIT+ Treg population is increased by at least 60%. In some embodiments, the increase of TIGIT expression on Tregs (FOXP3+, CD4+ T Cells) or the expansion of TIGIT+ Treg population is increased by at least 70%. In some embodiments, the increase of TIGIT expression on Tregs (FOXP3+, CD4+ T Cells) or the expansion of TIGIT+ Treg population is increased by at least 80%. In some embodiments, the increase of TIGIT expression on Tregs (FOXP3+, CD4+ T Cells) or the expansion of TIGIT+ Treg population is increased by at least 90%. In some embodiments, the increase of TIGIT expression on Tregs (FOXP3+, CD4+ T Cells) or the expansion of TIGIT+ Treg population is increased by at least 100%. In some embodiments, the increase of TIGIT expression on Tregs (FOXP3+, CD4+ T Cells) or the expansion of TIGIT+ Treg population is increased by at least 125%. In some embodiments, the increase of TIGIT expression on Tregs (FOXP3+, CD4+ T Cells) or the expansion of TIGIT+ Treg population is increased by at least 150%. In some embodiments, the increase of TIGIT expression on Tregs (FOXP3+, CD4+ T Cells) or the expansion of TIGIT+ Treg population is increased by at least 200%. In some embodiments, the increase of TIGIT expression on Tregs (FOXP3+, CD4+ T Cells) or the expansion of TIGIT+ Treg population is increased by at least 25% to about 200%. %. In some embodiments, the increase of TIGIT expression on Tregs (FOXP3+, CD4+ T Cells) or the expansion of TIGIT+ Treg population is increased by at least 25% to about 150%. In some embodiments, the increase of TIGIT expression on Tregs (FOXP3+, CD4+ T Cells) or the expansion of TIGIT+ Treg population is increased by at least 25% to about 100%. In some embodiments, the increase of TIGIT expression on Tregs (FOXP3+, CD4+ T Cells) or the expansion of TIGIT+ Treg population is increased by at least 50% to about 150%. In some embodiments, the increase of TIGIT expression on Tregs (FOXP3+, CD4+ T Cells) or the expansion of TIGIT+ Treg population is increased by at least 50% to about 100%. In some embodiments, the increase of TIGIT expression on Tregs (FOXP3+, CD4+ T Cells) or the expansion of TIGIT+ Treg population is increased by at least 100% to about 150%. In some embodiments, the increase of TIGIT expression on Tregs (FOXP3+, CD4+ T Cells) or the expansion of TIGIT+ Treg population is increased by at least 100% to about 200%.

As used herein, a cell is considered to be positive for a marker, such as, but not limited to FOXP3, CD4, CD69, TIGIT, and the like, if the cell expresses the protein. The expression of a protein can be determined by any method, such as, but not limited to flow cytometry, western blot, and the like.

The contacting or administration of the molecule (polypeptide or antibody) can occur, for example, by administration of the polypeptides provided for herein to a subject. The administration can be as provided for herein, such as but not limited to, intravenous or subcutaneous administration.

### Pharmaceutical Compositions and Kits

In some embodiments, the present embodiments provide compositions, e.g., pharmaceutically acceptable compositions, which include a therapeutic compound described herein, formulated together with a pharmaceutically acceptable carrier. As used herein, “pharmaceutically acceptable carrier” includes any and all solvents, dispersion media, isotonic and absorption delaying agents, and the like that are physiologically compatible.

The carrier can be suitable for intravenous, intramuscular, subcutaneous, parenteral, rectal, local, ophthalmic, topical, spinal or epidermal administration (e.g. by injection or infusion). As used herein, the term “carrier” means a diluent, adjuvant, or excipient with which a compound is administered. In some embodiments, pharmaceutical carriers can also be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used. The carriers can be used in pharmaceutical compositions comprising the therapeutic compounds provided for herein.

The compositions and compounds of the embodiments provided for herein may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In some embodiments, the mode of administration is parenteral (e.g., intravenous, subcutaneous, intraperitoneal, intramuscular). In some embodiments, the therapeutic molecule is administered by intravenous infusion or injection. In another embodiment, the therapeutic molecule is administered by intramuscular or subcutaneous injection. In another embodiment, the therapeutic molecule is administered locally, e.g., by injection, or topical application, to a target site. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The compositions typically should be sterile and stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic compound can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic compound is 0.1-30 mg/kg, more preferably 1-25 mg/kg. Dosages and therapeutic regimens of the therapeutic compound can be determined by a skilled artisan. In certain embodiments, the therapeutic compound is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the therapeutic compound is administered at a dose from about 10 to 20 mg/kg every other week. The therapeutic compound can be administered by intravenous infusion at a rate of more than 20 mg/min, e.g., 20-40 mg/min, and typically greater than or equal to 40 mg/min to reach a dose of about 35 to 440 mg/m2, typically about 70 to 310 mg/m2, and more typically, about 110 to 130 mg/m2. In embodiments, the infusion rate of about 110 to 130 mg/m2 achieves a level of about 3 mg/kg. In other embodiments, the therapeutic compound can be administered by intravenous infusion at a rate of less than 10 mg/min, e.g., less than or equal to 5 mg/min to reach a dose of about 1 to 100 mg/m2, e.g., about 5 to 50 mg/m2, about 7 to 25 mg/m2, or, about 10 mg/m2. In some embodiments, the therapeutic compound is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of a therapeutic molecule. A “therapeutically effective amount” refers to an amount effective, at dosages and for periods of time necessary, to achieve the desired therapeutic result. A therapeutically effective amount of a therapeutic molecule may vary according to factors such as the disease state, age, sex, and weight of the individual, and the ability of the therapeutic compound to elicit a desired response in the individual. A therapeutically effective amount is also one in which any toxic or detrimental effects of a therapeutic molecule t is outweighed by the therapeutically beneficial effects. A “therapeutically effective dosage” preferably inhibits a measurable parameter, e.g., immune attack at least about 20%, more preferably by at least about 40%, even more preferably by at least about 60%, and still more preferably by at least about 80% relative to untreated subjects. The ability of a compound to inhibit a measurable parameter, e.g., immune attack, can be evaluated in an animal model system predictive of efficacy in transplant rejection or autoimmune disorders. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the embodiments is a kit comprising a therapeutic compound described herein. The kit can include one or more other elements including: instructions for use; other reagents, e.g., a label, a therapeutic agent, or an agent useful for chelating, or otherwise coupling, a therapeutic molecule to a label or other therapeutic agent, or a radioprotective composition; devices or other materials for preparing the a therapeutic molecule for administration; pharmaceutically acceptable carriers; and devices or other materials for administration to a subject.

### EXAMPLES

The following examples are illustrative, but not limiting, of the compounds, compositions and methods described herein. Other suitable modifications and adaptations known to those skilled in the art are within the scope of the following embodiments.

Example 1: PD-1/LAG-3 and FcγRIIβ dual targeted polypeptide (i.e., targeting cells that express PD-1 and LAG-3 as well as an antibody that binds selectively to FcγRIIβ) Fc is used to treat autoimmune disorder. A polypeptide comprising an antibody that is a PD-1 agonist, an antibody that is a LAG-3 agonist, and a scFv that is selective for FcγRIIβ, which are linked with a Fc polypeptide with mutations that are selective for FcγRIIβ is administered to a subject with an autoimmune disorder and the subject is treated for the autoimmune disorder. Although this example describes a Fc polypeptide with mutations that are selective for FcγRIIβ, the Fc polypeptide may also not be selective, but could have the LALAGA mutations (AAA) as provided for herein.

Example 2: PD-1-FcγRIIβ dual targeted polypeptide is used to treat autoimmune disorder. A polypeptide comprising an antibody that is a PD-1 agonist, a scFv that is selective for FcγRIIβ, which are linked with a Fc polypeptide with mutations that are selective for FcγRIIβ is administered to a subject with an autoimmune disorder and the subject is treated for the autoimmune disorder. Although this example describes a Fc polypeptide with mutations that are selective for FcγRIIβ, the Fc polypeptide may also not be selective, but could have the LALAGA mutations (AAA) as provided for herein.

Example 3: PD-1-FcγRIIα dual targeted polypeptide is used to treat lung cancer. A polypeptide comprising an antibody that is a PD-1 antagonist, a scFv that is selective for FcγRIIα, which are linked with a Fc polypeptide with mutations that are selective for FcγRIIα is administered to a subject with lung cancer and the subject is treated for the autoimmune disorder. Although this example describes a Fc polypeptide with mutations that are selective for FcγRIIβ, the Fc polypeptide may also not be selective, but could have the LALAGA mutations (AAA) as provided for herein.

Example 4: PD-1/LAG-3 and FcγRIIα dual targeted polypeptide (i.e., targeting cells that express PD-1 and LAG-3 as well as an antibody that binds selectively to FcγRIIα) Fc is used to treat autoimmune disorder. A polypeptide comprising an antibody that is a PD-1 antagonist, an antibody that is a LAG-3 antagonist, and a scFv that is selective for FcγRIIα, which are linked with a Fc polypeptide with mutations that are selective for FcγRIIβ is administered to a subject with an autoimmune disorder and the subject is treated for the autoimmune disorder. Although this example describes a Fc polypeptide with mutations that are selective for FcγRIIβ, the Fc polypeptide may also not be selective, but could have the LALAGA mutations (AAA) as provided for herein.

Example 5: PD-1 or LAG-3, and FcγRIIβ dual targeted polypeptide (i.e., targeting cells that express PD-1 or LAG-3 as well as an antibody that binds selectively to FcγRIIβ) Fc is used to treat autoimmune disorder. A polypeptide comprising an antibody that is a PD-1 agonist, or an antibody that is a LAG-3 agonist, and a scFv that is selective for FcγRIIβ, which are linked with a Fc polypeptide with mutations that are selective for FcγRIIβ is administered to a subject with an autoimmune disorder and the subject is treated for the autoimmune disorder. Although this example describes a Fc polypeptide with mutations that are selective for FcγRIIβ, the Fc polypeptide may also not be selective, but could have the LALAGA mutations (AAA) as provided for herein.

Example 6: PD-1 or LAG-3, and FcγRIIα dual targeted polypeptide (i.e., targeting cells that express PD-1 or LAG-3 as well as an antibody that binds selectively to FcγRIIα) Fc is used to treat autoimmune disorder. A polypeptide comprising an antibody that is a PD-1 antagonist, or an antibody that is a LAG-3 antagonist, and a scFv that is selective for FcγRIIα, which are linked with a Fc polypeptide with mutations that are selective for FcγRIIβ is administered to a subject with an autoimmune disorder and the subject is treated for the autoimmune disorder. Although this example describes a Fc polypeptide with mutations that are selective for FcγRIIβ, the Fc polypeptide may also not be selective, but could have the LALAGA mutations (AAA) as provided for herein.

Example 7: LAG-3-FcγRIIβ dual targeted polypeptide is used to treat autoimmune disorder. A polypeptide comprising an antibody that is a LAG-3 agonist, a scFv that is selective for FcγRIIβ, which are linked with a Fc polypeptide with mutations that are selective for FcγRIIβ is administered to a subject with an autoimmune disorder and the subject is treated for the autoimmune disorder. Although this example describes a Fc polypeptide with mutations that are selective for FcγRIIβ, the Fc polypeptide may also not be selective, but could have the LALAGA mutations (AAA) as provided for herein.

Example 8: LAG-3-FcγRIIα dual targeted polypeptide is used to treat lung cancer. A polypeptide comprising an antibody that is a LAG-3 antagonist, a scFv that is selective for FcγRIIα, which are linked with a Fc polypeptide with mutations that are selective for FcγRIIα is administered to a subject with lung cancer and the subject is treated for the autoimmune disorder. Although this example describes a Fc polypeptide with mutations that are selective for FcγRIIβ, the Fc polypeptide may also not be selective, but could have the LALAGA mutations (AAA) as provided for herein.

Example 9: PD1/LAG-3-FcγRIIβ dual targeted polypeptide (i.e., targeting cells that express PD-1 and LAG-3 as well as an antibody that binds selectively to FcγRIIβ) is used to treat autoimmune disorder. A polypeptide comprising an antibody that is a PD-1 agonist, an antibody that is a LAG-3 agonist, and an scFv that is selective for FcγRIIβ, which are linked with a Fc polypeptide with mutations that are selective for FcγRIIβ is administered to a subject with an autoimmune disorder and the subject is treated for the autoimmune disorder. Although this example describes a Fc polypeptide with mutations that are selective for FcγRIIβ, the Fc polypeptide may also not be selective, but could have the LALAGA mutations (AAA) as provided for herein.

Example 10: PD1/LAG-3-FcγRIIα dual targeted polypeptide (i.e., targeting cells that express PD-1 and LAG-3 as well as an antibody that binds selectively to FcγRIIβ) is used to treat lung cancer. A polypeptide comprising an antibody that is a PD-1 antagonist, an antibody that is a LAG-3 antagonist, and an scFv that is selective for FcγRIIα, which are linked with a Fc polypeptide with mutations that are selective for FcγRIIα is administered to a subject with lung cancer and the subject is treated for the autoimmune disorder. Although this example describes a Fc polypeptide with mutations that are selective for FcγRIIα, the Fc polypeptide may also not be selective, but could have the LALAGA mutations (AAA) as provided for herein.

Example 11: Benchmark anti-PD-1 and anti-LAG3 agonist antibody variable domains were used for prototype generation, and anti-RSV variable domains were used as negative control. The variable domains were fused to a panel of benchmark Fc domains with different levels of selectivity: Xencor “SELF” mutant, Chugai “V12” and P238D mutants in addition to IgG1 wild-type Fc and “AAA” low/no FcR binding Fc. Benchmark moieties included IgG1-Fc (WT), IgG1-Fc V12 (V12) which exhibits increased selectivity and affinity for FcγRIIβ, IgG1-Fc P238D (P238D) which is selected for FcγRIIβ, IgG1-Fc S276E L328F (SELF) which exhibits increase affinity but not selectivity for FcγRIIβ, and IgG1-Fc AAA (AAA) which is Fc binding silent.

Example 12: Purified prototype antibody test articles were generated for binding and functional assays. Said test articles were expressed in Expi293F cells. Each antibody test article was purified by passing it through a 5 mL PrismA column. Target antibodies were eluted with 0.1M Glycine at pH 2.8, and neutralized immediately using 5% 1M Tris HCl at pH 8.0. The eluted samples were loaded to analytical SEC to check for monodispersity of Protein of Interest (POI). All test articles were purified with the majority population present as monomers, as shown in Table 15 below.

TABLE 15

| Test Articles | monomeric POI (%) | Yield (mg/L) |
|---|---|---|
| anti PD1 IgG1 WT | 95 | ~98 |
| anti PD1 IgG1 P238D | 96 | ~97 |
| anti PD1 IgG1 V12 | 96 | ~96 |
| anti-RSV WT | 96 | ~145 |
| anti-RSV V12 | 99 | ~106 |
| Anti-LAG3 SELF | 93 | ~72 |

Example 13: Binding between test antibodies and Fc gamma receptors, or PD-1 receptors, was analyzed using Carterra SPR. In brief, antibodies were captured on protein A/G chips at concentrations of 10 μg/mL, 1 μg/mL, and 0.1 μg/mL (in duplicates). Each solution analyte protein was injected at 5 μM for Fc gamma receptors, or at 0.5 μM for PD-1 receptors. PD-1 was used to confirm antibody integrity. Binding kinetics for each antibody against PD-1, human and monkey FcγRIIβ and FcγRIIα, including human FcγRIIα-R167 and FcγRIIα-H167, were obtained. Test antibodies showed the following affinities: (1) anti-PD1 IgG1 V12 showed a KD (nM) of 2.0 to human PD-1, KD of 11 to cyno PD-1, KD of 1.8 to human FcγRIIα, and a KD of 0.03 to human FcγRIIβ. Effectively, the anti-PD1 IgG1 V12 molecule binds to both PD-1 and FcγRII receptors.

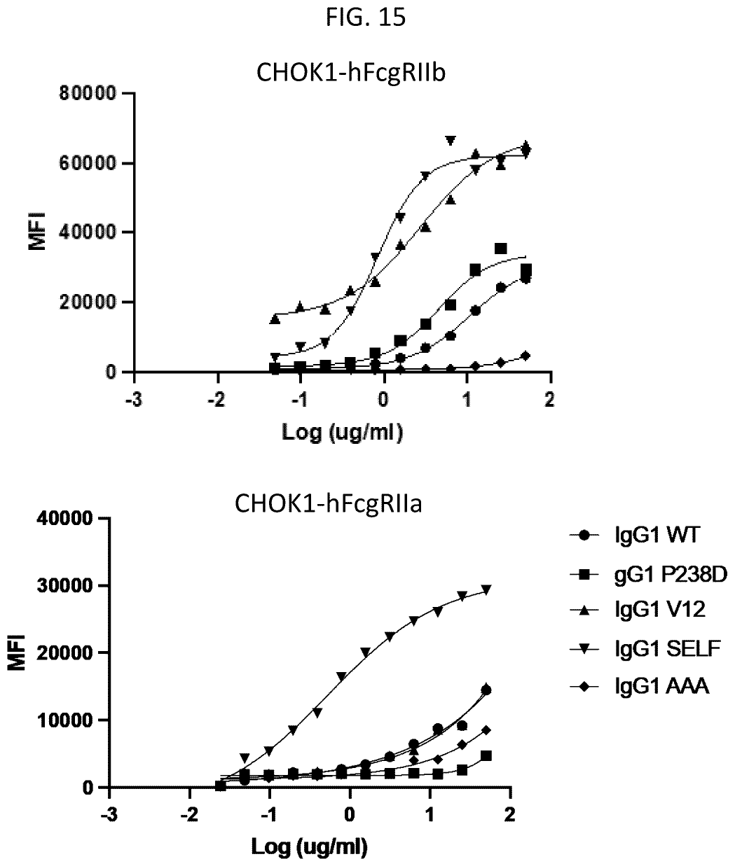

Example 14: Binding of antibodies with different affinities to Fc gamma receptors was measured using flow cytometry. In brief, CHOK1 lines overexpressing either FcγRIIβ or FcγRIIα (R131) were detached, resuspended in phosphate buffered saline (PBS) 3% FBS and incubated for 30 minutes at 4° C. with test articles (1:2 serial dilution, 11 points dilution starting from 50 μg total protein). Next, cells were washed and incubated for additional 30 minutes at 4° C. with a directly conjugated antibody recognizing the human kappa chain of the test articles. Cells were then washed, resuspended in fixation buffer for 1 hour at 4° C., washed again and resuspended in PBS prior to flow cytometry. Binding curves (logEC50) for each antibody against human FcγRIIβ or FcγRIIα (R131) were obtained, and are shown in FIG. 15. EC50 values are also shown in Table 16 below.

TABLE 16

| | Log EC50 | Log EC50 |
|---|---|---|
| Test Articles | (FcγR2IIβ) | (FcγR2IIα) |
| anti PD1 IgG1 WT | 1.026 | 9.376 |
| anti PD1 IgG1 P238D | 0.6485 | 3.438 |
| anti PD1 IgG1 V12 | 0.437 | 7.822 |
| anti-LAG3 SELF | −0.0779 | −0.2704 |
| anti-RSV AAA | 4.425 | 3.903 |

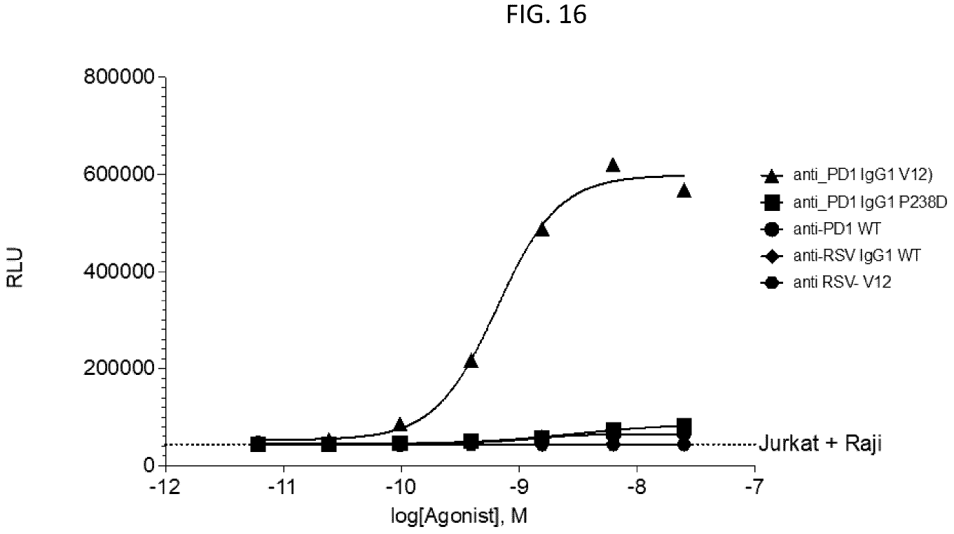

Example 15: A reporter cell line that measures fluorescence derived from SHP2 recruitment to PD1 was used as a proxy of PD-1 agonism. In brief, Raji B cells were removed from cell culture, resuspended in cell plating reagent with 3% FBS and incubated for 1 hour at 37° C. with 100 nM to 0.006 nM of test articles. Jurkat PD-1 (SHP2) reporter cells were removed from cell culture, resuspended in cell plating reagent with 3% FBS, and incubated with the Raji cells with test articles for an additional 2 hours at room temperature. Detection reagents were added to each well and luminescence was read using a plate reader. Agonism produced in reporter cell lines was enhanced by antibodies with greater affinities to FcγRIIβ over the wild-type antibody control, as shown in FIG. 16 and in Table 17 below.

TABLE 17

| Test Articles | Log EC50 (SHP2 recruitment) |
|---|---|
| anti PD1 IgG1 WT | −8.911 |
| anti PD1 IgG1 P238D | −8.501 |
| anti PD1 IgG1 V12 | −9.187 |
| anti-RSV-WT | Not measurable |
| anti-RSV-V12 | Not measurable |

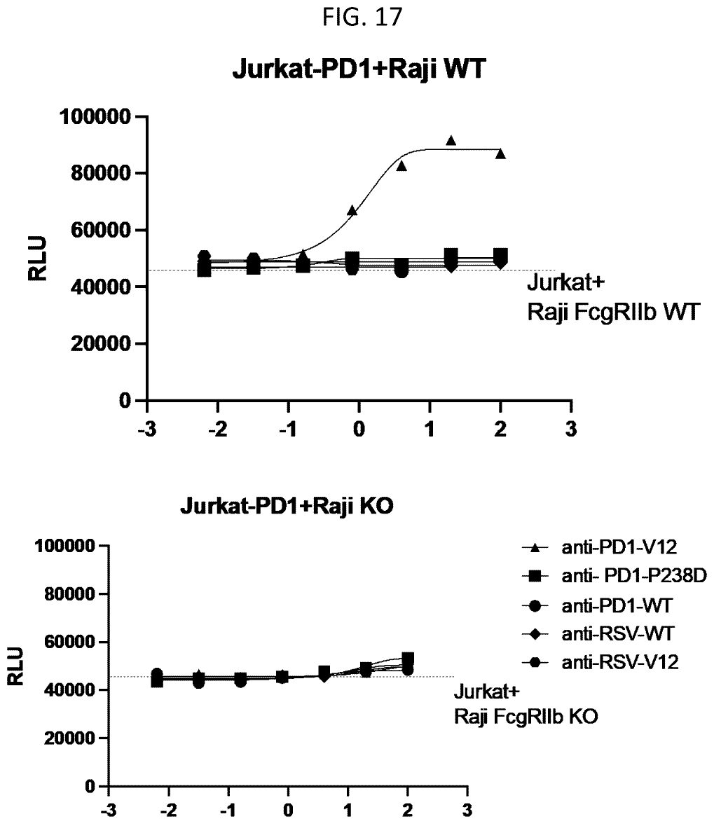

Example 16: Next, it was assessed whether the enhanced PD1 agonism observed with high affinity antibodies for FcγRIIβ is dependent on FcγRIIβ expression. Raji B cells expressing or deficient of FcγRIIβ, and Jurkat-PD1 (SHP-2) reporter cells were removed from cell culture, resuspended in cell plating reagent with 3% FBS and incubated for 3 hour at 37° C. with 100 nM to 0.006 nM of test articles. Detection reagents were added to each well and luminescence was read using a plate reader. Agonism produced in reporter cell lines was enhanced by antibodies with greater affinities to FcγRIIβ over the wild-type antibody control, as shown in FIG. 17 and in Table 18 below. Agonism was completely abrogated in absence of FcγRIIβ. Accordingly, superior inhibitory checkpoint receptor agonism on the T cell reporter line is mediated through selective binding of Fc to FcγRIIβ.

TABLE 18

| | JURKAT-PD1 + | JURKAT-PD1 + |
|---|---|---|
| | RAJI WT | RAJI FcγRIIβ |
| | (FcγRIIβ+) | Knock Out |
| Test Articles | Log EC50 | Log EC50 |
| | (SHP2 recruitment) | (SHP2 recruitment) |
| anti PD1 IgG1 WT | 19.71 | Not measurable |
| anti PD1 IgG1 P238D | 0.1946 | Not measurable |
| anti PD1 IgG1 V12 | −11.06 | Not measurable |
| anti-RSV-WT | 0.01219 | Not measurable |
| anti-RSV-V12 | Not measurable | Not measurable |

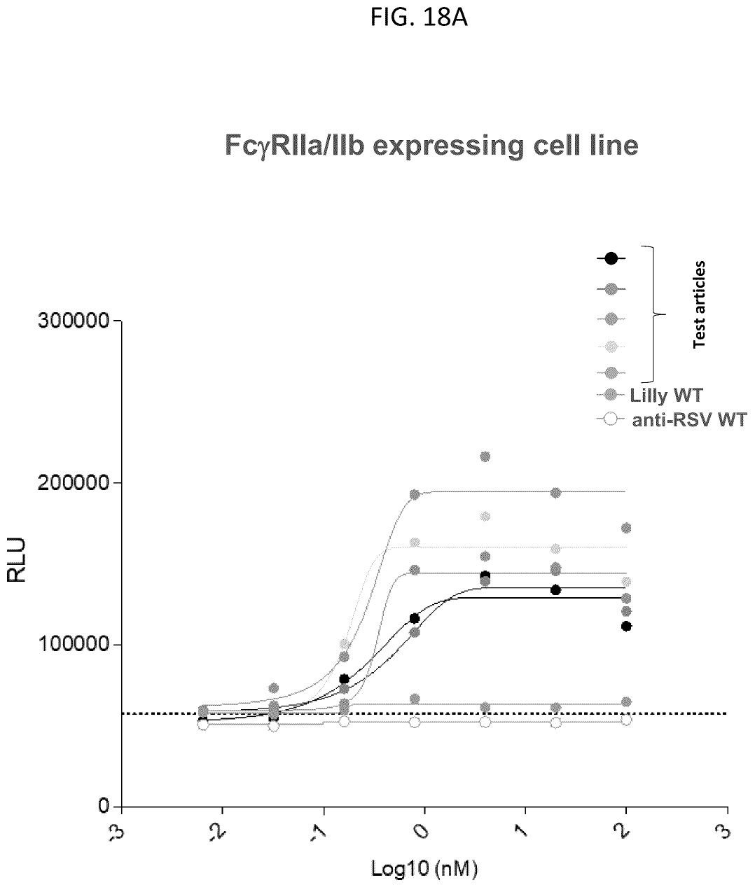

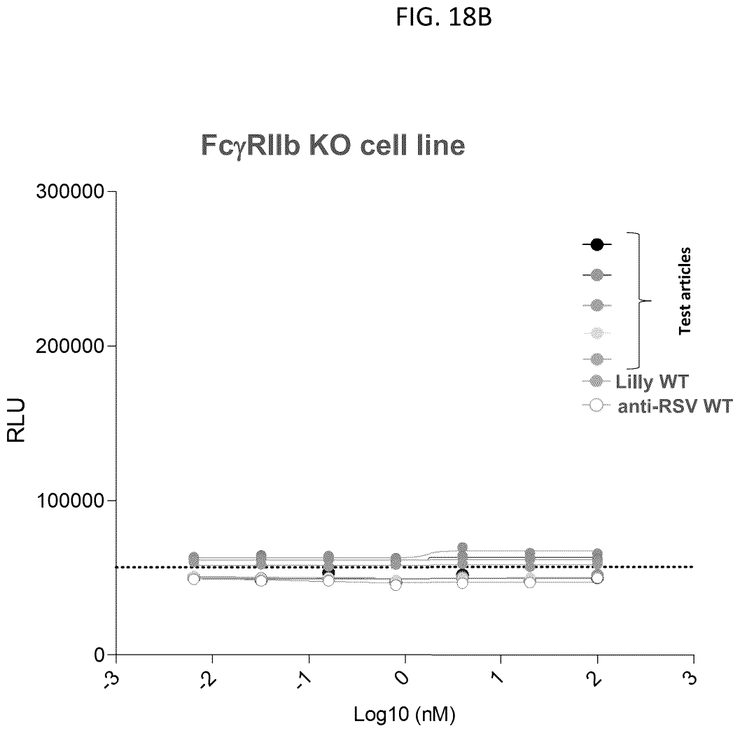

Example 17: PD-1 agonism by selective anchoring to FcγRIIβ was next assessed. Daudi cells expressing FcγRIIβ and FcγRIIα or daudi cells expressing only FcγRIIα were used as anchoring cells. Jurkat-PD1 (SHP-2) reporter cells were used to measure PD-1 agonism. Test articles comprised prototype anti-PD-1 moiety linked to an effectorless Fc polypeptide on one terminal of the Fc, and FcγRIIβ selective scFv moiety linked to the effectorless Fc polypeptide on the other terminal of the Fc. Anti-RSV and anti-PD1 (Lilly) antibodies were used as controls. Agonism produced in reporter cell lines was enhanced by antibodies with scFv moieties having affinity for FcγRIIβ, as shown in FIG. 18A. Agonism was completely abrogated in absence of FcγRIIβ as shown in FIG. 18B. Accordingly, PD-1 agonism was achieved by selective anchoring to FcγRIIβ via the scFv moiety.

Example 18: Expi293F cells were transfected to transiently express anti-FcγRIIβ antibodies. Each anti-FcγRIIβ antibody, or antigen-binding fragment thereof, was purified by passing through 1 ml PrismA columns. Target antibodies were eluted with 0.1M Glycine at pH 2.8, and neutralized immediately with 5% of 1M Tris HCl at pH 8. The eluted samples were loaded to analytical SEC to assess monodispersity of Protein of Interest (Pol). Accordingly, all test articles were purified with majority population as monomers on analytical SEC, as shown in the Table 19 below.

TABLE 19

| mAb | monomeric POI (% ) | Yield (mg/L) |
|---|---|---|
| TA25 | 96.5 | 29.6 |
| TA26 | 86.3 | 63.6 |
| TA28 | 89.7 | 13 |
| TA30 | 71.8 | 22.7 |
| TA36 | 78.2 | 62.4 |
| TA39 | 85.2 | 21.2 |

Example 19: Anti-FcγRIIβ antibodies were captured on AHC biosensors at a concentration of 5 μg/mL. Antigens (FcγRIIα/FcγRIIβ) were serially diluted two-fold with a starting concentration of 5u M. The biosensors with immobilized antibodies were dipped into the different antigen concentrations and kinetics data were collected. The binding kinetics (KD affinity, Kon association rate, and Koff dissociation rates for each antibody against human FcγRIIβ and FcγRIIα show selectivity of tested antibodies for FcγRIIβ over FcγRIIα.

TABLE 20

| | huFcγRIIβ | huFcγRIIα | |
|---|---|---|---|
| | Affinity | (R131) Affinity | Fold |
| Name | K_{D} (nM) | K_{D} (nM) | selectivity |
| TA25 | 122.0 | 114000 | 934 |
| TA26 | 50.2 | 715 | 14 |
| TA28 | 11.0 | 623 | 56 |
| TA36 | 45.0 | 141 | 3 |
| TA39 | 30.3 | 115 | 4 |

Example 20: Anti-FcγRIIβ antibodies were captured on AHC biosensors at a concentration of 5 μg/mL. Antigens (FcγRI or FcγRIIIα) were serially diluted two-fold with a starting concentration of 5 μM. The biosensors with immobilized antibodies were dipped into the different antigen concentrations and kinetics data were collected. The binding kinetics (KD affinity, Kon association rate, and Koff dissociation rates) for each antibody against human FcγRI or FcγRIIIα show that these antibodies do not bind to FcγRI or FcγRIIIα.

TABLE 21
FcRN Binding

| Construct | KD (nM) | Ka (1/Ms) | Kdis (1/s) |
|---|---|---|---|
| TA | 180 | 3.56E+05 | 6.39E−02 |
| TA25 | 231 | 3.41E+05 | 7.88E−02 |
| TA26 | 236 | 4.03E+05 | 9.51E−02 |
| TA28 | 273 | 3.78E+05 | 1.03E−01 |
| TA36 | 289 | 4.07E+05 | 1.18E−01 |
| TA39 | 250 | 4.00E+05 | 1.00E−01 |

Example 21: Anti-FcγRIIβ antibodies were captured on protein A/G chips at 10 ug/mL, 1 ug/mL and 0.1 ug/mL concentrations (in duplicates). Each analyte binder was injected at 7 concentrations with 5-fold serial dilutions, with the starting concentration for FcγRs at 5 uM, and for PD-1 at 0.5 uM. PD-1 was used to confirmed antibody integrity. Kinetics data was subsequently collected. The binding kinetics (KD affinity, Kon association rate, Koff dissociation rates) for each antibody against PD-1, human and monkey FcγRIIβ and FcγRIIα, including human FcγRIIα-R167 and FcγRIIα_H167, showed binding of FcγRIIβ selective antibodies to Hu/Cy FcγRIIβ/IIα.

TABLE 22

| Sample ID | huR2b | huR2a | CyR2b | CyR2a |
|---|---|---|---|---|
| TA25 | 2.5348E−07 | 6.0836E−06 | 1.497E−06 | 7.7692E−07 |
| TA26 | 1.5393E−07 | 6.5136E−06 | 3.1335E−06 | 2.4819E−06 |
| TA30 | 5.611E−08 | 6.3323E−07 | 1.2083E−06 | 1.1201E−06 |
| TA36 | 2.9895E−07 | 4.0639E−06 | 3.6618E−06 | 2.2968E−06 |

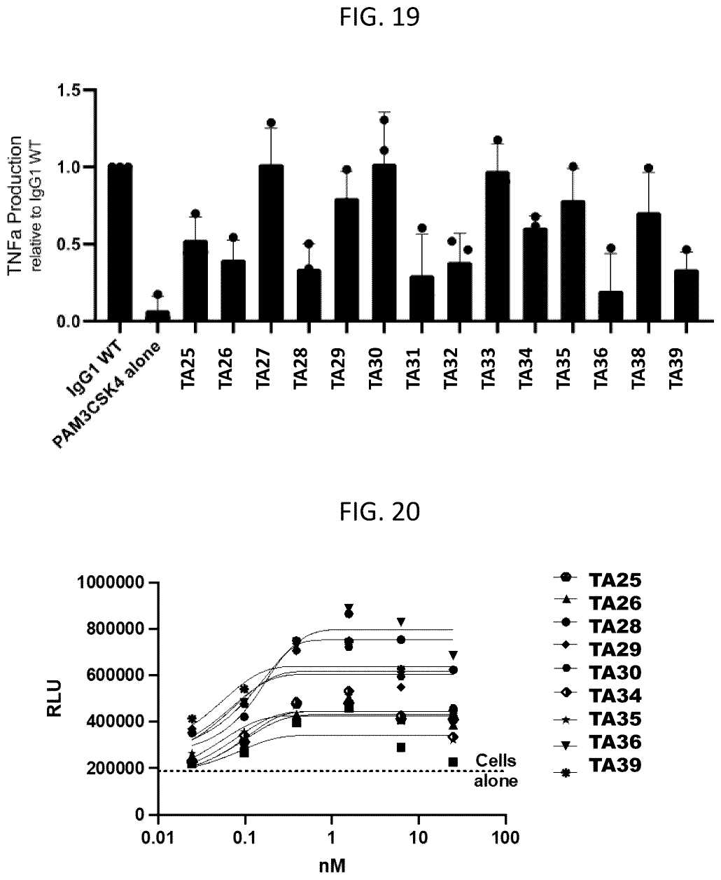

Example 22: Anti-FcγRIIβ antibodies fused to Fc molecules at 2 μg/mL were coated in PBS for 1 hour at room temperature. Plate is washed and blocked with PBS 10% FBS for 1 hour at 37° C. MoDCs with the addition of 10 μg/mL of PAM3CSK4 in RPMI were added to a coated plate. Supernatant was then collected at 24 hours and TNFa was measured by MSD. As illustrated in FIG. 19, anti-FcγRIIβ antibodies fused to Fc molecules showed reduced TNFa production relative to IgG1 wild-type.

Example 23:293 cells expressing FcγRIIβ and FcγRIIα were removed from cell culture, resuspended in cell plating reagent with 10% FBS and incubated for 4 hours at 37° C. with (100 nM to 0.002 nM) of test articles and in co-culture with Jurkat PD-1 (SHP2) reporter cells. Detection reagents were added to each well and luminescence was detected using a plate reader. Increase in luminescence (RLU) as result of SHP2 recruitment allowed for identification of PD-1 agonists, as illustrated in FIG. 20.

Example 24: In-silico docking was performed to build Fc-FcγRIIα with kinked hinge with PDB 3WJJ and PDB 1H9V. In-silico free energy calculations were performed for all publicly available Fc mutants with reported affinities to build a machine learning model for predicting mutation effects on the affinities. In-silico free energy calculations were performed for all possible point mutations near the interfaces between Fc and receptors and predict the affinities and selectivity. Evolutionary sequence modeling to predict mutation effects on Fc stability was performed. Mutation libraries were constructed, selectivity and evolutionary scores were co-optimized separately and top mutations and interfaces were combined. A number of mutations were introduced around residues P238 as alternatives to P238D at interface 1: P238E, P238F, P238N, P238Q, P238M. The model favored D/E mutations for the near contact residues to Y205: A327D and A330E. Additional close G237 was also identified with candidates: G237H, G237M and G237D, and G237E were also included due to proximity to Y205 and preferred D/E mutations to enhance selectivity.

Example 25: In-silico docking was performed to build Fc-FcγRIIα with kinked hinge with PDB 3WJJ and PDB 1H9V. In-silico free energy calculations were performed for all publicly available Fc mutants with reported affinities to build a machine learning model for predicting mutation effects on the affinities. In-silico free energy calculations were performed for all possible point mutations near the interfaces between Fc and receptors and predict the affinities and selectivity. Evolutionary sequence modeling was performed to predict mutation effects on Fc stability. Mutation libraries were constructed, selectivity and evolutionary scores for interface 2 were co-optimized separately and top mutations and interfaces were combined. A number of mutations of interest were found near S177 of FcγRIIβ within 10 A: E269Y, D270E, T299F, D265F, forming hydrogen bonds. Y269N mutation targeted K172 of FcγRIIβ within 7 A to enhanced binding affinity. H268Q mutation had superior stability predicted from evolutionary model.

Example 26: In-silico docking was performed to build Fc-FcγRIIα and Fc-FcγRIIβ with normal hinge with PDB 1E4K. In-silico free energy calculations were performed for all publicly available Fc mutants with reported affinities to build a machine learning model for predicting mutation effects on the affinities. In-silico free energy calculations were performed for all possible point mutations near the interfaces between Fc and receptors and predict the affinities and selectivity. Evolutionary sequence modeling was performed to predict mutation effects on Fc stability. Mutation libraries were constructed, selectivity and evolutionary scores for interface 1 were co-optimized separately and top mutations and interfaces were combined. L235G mutation showed upstream flexibility and G236Q mutation showed polar engagement to Y205 at interface 1 within 5A. P329R mutation showed backbone change with long polar residue, selectivity/affinity boosted by G237R with same reason at interface 1. A327D mutation targeted —OH of S177 within 5A at interface 2. S298Q mutation targeted K172 within 4A for enhanced affinity. Upstream mutations of L234M, G236P, G237L created nonpolar local environment to boost A327D affinity to S177.

Example 27: The variant IgG Fc polypeptides described herein were transiently expressed in Expi293F cells. The variant IgG Fc polypeptides were purified by passing through PrismA resins. Target antibodies were eluted with buffer 0.1M Glycine, pH 2.7. The flow through and prismA eluted samples were loaded to CE-SDS to check purification result. All variants were purified with majority population as monomers on analytical SEC.

Example 28: Antibodies comprising variant IgG Fc polypeptides described herein were captured on a protein A/G chip at 10 μg/mL, 1 μg/mL, and 0.1 ug/mL concentrations (in duplicates). Each analyte binder was injected at seven concentrations with 5-fold serial dilutions, and kinetics data was collected. Binding kinetics (KD affinity, Kon association rate, Koff dissociation rate) were collected. Affinity KD of each Fc variant against human FcγRIIα_R167, FcγRIIα_H167, FcγRIIβ, and cyno FcγRIIα and FcγRIIβ was described in Table 23 below. Due to the limitation of affinity measurement, KD values were categorized as “no binding” when no response was observed, “>5 uM” when KD is weaker than 5 uM, and KD was measured if the KD was stronger than 5 uM. For IgG1 WT, KD of 5.1 uM is shown. This value was consistent with previous reports.

TABLE 23

| | ID | | | | | | | | | | |
|---|---|---|---|---|---|---|---|---|---|---|---|
| | | | | | | | | | | Ratio IIα/IIβ | Ratio IIβ/IIα |
| | Hu FcγRIIA_R131 | | | Hu FcγRIIA_H131 | | | Hu FcγRIIβ | | | (KD human | (KD human |
| | KD | SD | | KD | SD | | KD | SD | | IIα R/KD | IIβ/KD |
| | (M) | (M) | Fold | (M) | (M) | Fold | (M) | (M) | Fold | human IIβ) | human IIα R) |
| IgG1 | 1.55E−06 | 2.12E−07 | 1.00 | 1.00E−06 | 7.56E−08 | 1.00 | 5.10E−06 | 9.52E−08 | 1.00 | 0.30 | 3.29 |
| WT | | | | | | | | | | | |
| IgG1 | >5 μM | N/A | <0.3 | No | N/A | N/A | 1.60E−06 | 7.55E−08 | 3.18 | 3.12 | 0.32 |
| P238D | | | | binding | | | | | | | |
| IgG1 | 1.80E−06 | 7.07E−07 | 0.86 | 3.35E−06 | 1.56E−06 | 0.30 | 3.10E−08 | 1.38E−10 | 164.35 | 57.97 | 0.02 |
| V12 | | | | | | | | | | | |
| AB-3 | >5 μM | N/A | <0.3 | 2.58E−06 | 1.84E−07 | 0.39 | 3.38E−06 | 6.86E−07 | 1.51 | 1.48 | 0.68 |
| AB-4 | No | N/A | N/A | No | N/A | N/A | 3.72E−06 | 8.66E−07 | 1.37 | >10 | <0.1 |
| | binding | | | binding | | | | | | | |
| AB-10 | No | N/A | N/A | No | N/A | N/A | 6.12E−06 | 3.08E−07 | 0.83 | >10 | <0.1 |
| | binding | | | binding | | | | | | | |
| AB-11 | >5 μM | N/A | <0.3 | >5 μM | N/A | <0.2 | 1.74E−06 | 2.60E−07 | 2.93 | 2.87 | 0.35 |
| AB-12 | >5 μM | N/A | <0.3 | 2.32E−06 | 7.31E−10 | 0.43 | 2.74E−07 | 5.24E−09 | 18.64 | 18.26 | 0.05 |
| AB-18 | >5 μM | N/A | N/A | >5 μM | N/A | <0.2 | 3.89E−06 | 1.05E−07 | 1.31 | 1.29 | 0.78 |
| AB-19 | 2.20E−06 | 5.66E−07 | 0.70 | 2.23E−06 | 3.82E−07 | 0.45 | 1.55E−06 | 3.54E−07 | 3.29 | 1.42 | 0.70 |
| AB-23 | >5 μM | N/A | <0.3 | No | N/A | N/A | 2.03E−06 | 5.72E−08 | 2.51 | 2.46 | 0.41 |
| | | | | binding | | | | | | | |
| AB-24 | >5 μM | N/A | <0.3 | >5 μM | N/A | <0.2 | 4.18E−07 | 1.74E−08 | 12.20 | 11.95 | 0.08 |

Ratio (IIα/IIβ) = KD(IIα)/KD(IIβ) (higher means stronger IIβ selectivity).
Fold = KD(IgG_WT)/KD(variants) (higher number means stronger Fcγ receptor affinity).
Ratio (IIβ/IIα) = KD(IIβ)/KD(IIα) (lower means stronger IIβ selectivity).
Compared to IgG1 wild type, the FcγRIIβ selective clones may have stronger human FcγRIIβ binding, weaker human FcγRIIα binding, or both Ila & IIβ at the same time.

Example 29: CHOK1 lines overexpressing either FcγRIIβ or IIα (R131) were detached, resuspended in PBS 3% FBS and incubated for 30 minutes at 4° C. with 40 μg/mL of test articles. Cells were washed and incubated for additional 30 minutes at 4° C. with a detection antibody (BV421 conjugated) recognizing the human kappa chain of our test articles (Cat #316518). Cells were further washed, resuspend in fixation buffer (cat number) for 1 hour, then washed and resuspended in PBS before their acquisition at the flow cytometer. Binding curves (EC50) for each antibody against human FcγRIIβ and FcγRIIα (R131) were obtained, and are shown below in Table 24.

TABLE 24

| | IIβ | IIα | IIα/IIβ |
|---|---|---|---|
| IgG1 P238D | 0.4 | −32.0 | −77.8 |
| IgG1 WT | 0.8 | −0.6 | −0.7 |
| IgG1 V12 | −0.1 | −0.4 | 5.5 |
| AB-12 | −0.2 | −0.5 | 3.3 |
| AB-18 | 0.0 | −1.0 | −52.4 |
| AB-23 | 0.3 | −57.5 | −209.6 |
| AB-24 | 0.0 | −1.3 | 27.6 |

The data illustrated in Table 24 shows that the variant IgG Fc polypeptides have comparable EC50 values to the IgG1 P238D molecule.

Example 30: Raji B cells were removed from cell culture, resuspended in cell plating reagent with 3% FBS and incubated for 1 hour at 37° C. with (100 nM to 0.006 nM) of test articles. Jurkat PD-1 (SHP2) reporter cells were removed from cell culture, resuspended in cell plating reagent with 3% FBS, and incubated with the Raji cells with test articles for an additional 2 hours at room temperature. Detection reagents were added to each well and luminescence was read using a plate reader. Agonism produced in reporter cell lines was enhanced by antibodies with greater affinities to FcγRIIβ over the wild-type antibody control.

Example 31: Expi293F cells were transfected to transiently express anti-PD-1 antibodies. Each anti-PD-1 antibody, or antigen-binding fragment thereof, was purified by passing through 5 ml PrismA columns. Target antibodies were eluted with 0.1M Glycine at pH 2.8, and neutralized immediately with 5% of 1M Tris HCl at pH 8. The eluted samples were loaded to analytical SEC to assess monodispersity of Protein of Interest (Pol). Accordingly, all test articles were purified with majority population as monomers on analytical SEC, as shown in the Table 25 below.

TABLE 25

| Test Articles | POI (%) or % monomer | Yield (mg/L) |
|---|---|---|
| TA98 | 98 | ~24 |
| TA132 | 100 | ~10 |
| TA126 | 99 | ~76 |
| TA128 | 99 | ~132 |
| TA119 | 99 | ~79 |
| TA78 | 96 | ~214 |
| TA259 | 84 | ~69 |
| TA335 | 95 | ~200 |

Example 32: Anti-PD-1 antibodies were captured on anti-human Fc biosensors at a concentration of 5 μg/mL. Antigens (huPD1 or cyPD1) were serially diluted two-fold with a starting concentration of 5u M. The biosensors with immobilized antibodies were dipped into the different antigen concentrations and kinetics data were collected. The binding kinetics (KD affinity, Kon association rate, and Koff dissociation rates for each antibody are shown in Table 26 below.

TABLE 26

| | huPD1 Binding Affinity | | | cyPD1 Binding | | |
|---|---|---|---|---|---|---|
| | and Kinetics | | | Affinity and Kinetics | | |
| Sample | K_{D} | kₒₙ | k_{off} | KD | kₒₙ | k_{off} |
| ID | (M) | (1/Ms) | (1/s) | (M) | (1/Ms) | (1/s) |
| TA259 | 399.0E−09 | 5.6E+05 | 2.2E−01 | 718.0 | 3.7E+05 | 2.7E−01 |
| TA78 | 4.1E−09 | 9.6E+08 | 3.9E+00 | 9.7 | 7.1E+08 | 6.9E+00 |
| TA98 | 258.0E−09 | 2.2E+06 | 5.7E−01 | 591.0 | 9.0E+05 | 5.3E−01 |
| TA101 | 32.9E−09 | 6.8E+05 | 2.2E−02 | 62.2 | 1.2E+05 | 7.6E−03 |
| TA102 | 24.8E−09 | 5.4E+05 | 1.4E−02 | 7.2 | 1.4E+06 | 1.0E−02 |
| TA104 | 33.1E−09 | 7.1E−05 | 2.4E−02 | 56.8 | 1.6E+05 | 9.0E−03 |
| TA275 | 600.0E−09 | 3.3E+05 | 2.0E−01 | 987.0 | 2.0E+05 | 2.0E−01 |
| TA289 | 484.0E−09 | 3.4E+06 | 1.6E+00 | 1390.0 | 1.6E+06 | 2.1E+00 |
| TA132 | 5.721E−09 | 1.131E+05 | 6.473E−04 | 9.201E−08 | 1.491E+05 | 1.372E−02 |

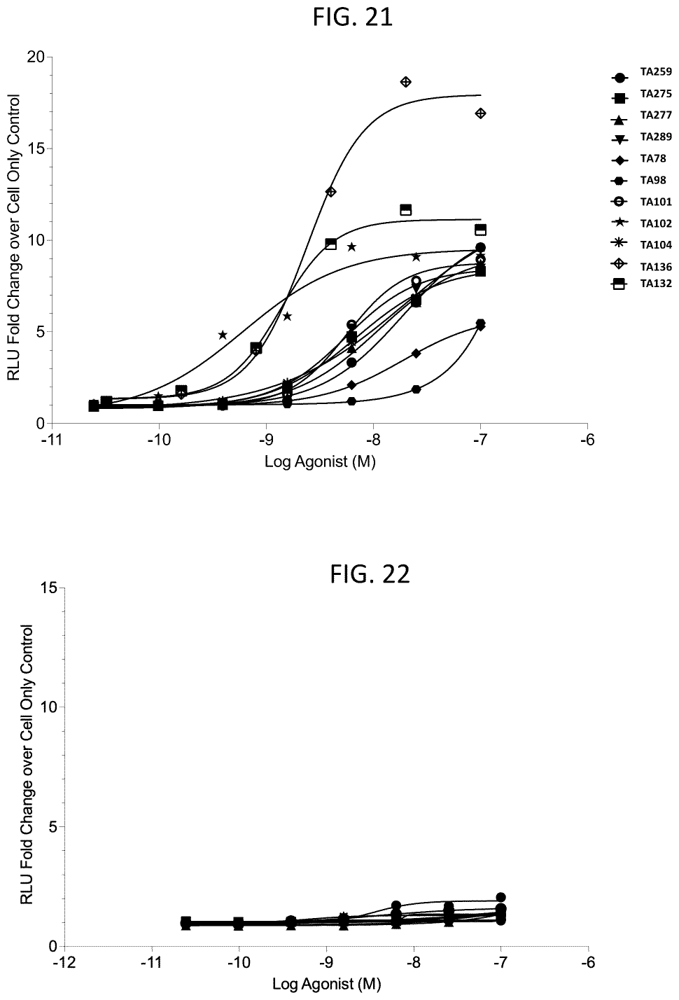

Example 33: Raji B cells were removed from cell culture, resuspended in cell plating reagent with 3% FBS and incubated for 1 hour at 37° C. with (100 nM to 0.006 nM) of test articles. Jurkat PD-1 (SHP2) reporter cells were removed from cell culture, resuspended in cell plating reagent with 3% FBS, and incubated with the Raji cells with test articles for an additional 2 hours at room temperature. Detection reagents were added to each well and luminescence was read using a plate reader. Agonism produced in reporter cell lines was enhanced by antibodies fused to an Fc as shown in FIG. 21.

Example 34: Raji B cells deficient in FcγRIIβ (FcγRIIβ knock-out) were removed from cell culture, resuspended in cell plating reagent with 3% FBS and incubated for 1 hour at 37° C. with (100 nM to 0.006 nM) of test articles. Jurkat PD-1 (SHP2) reporter cells were removed from cell culture, resuspended in cell plating reagent with 3% FBS, and incubated with the Raji cells with test articles for an additional 2 hours at room temperature. Detection reagents were added to each well and luminescence was read using a plate reader. Agonism produced in reporter cell lines was not observed by antibodies not fused to an Fc as shown in FIG. 22.

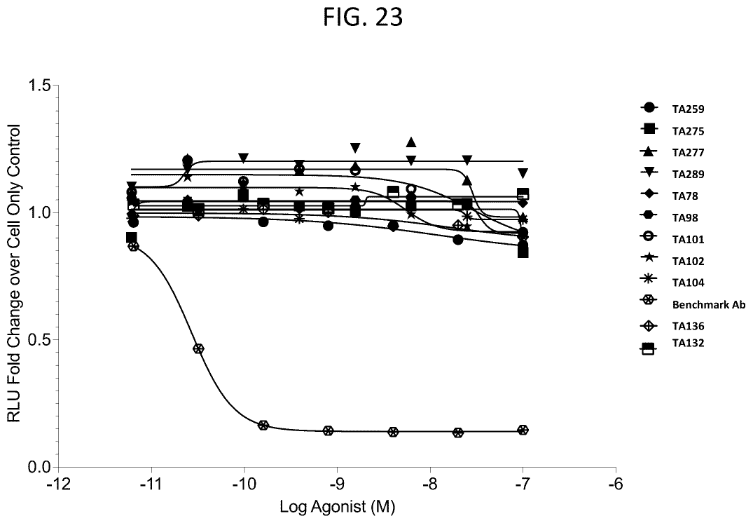

Example 35: U2OS-PDL1 were removed from cell culture, resuspended in cell plating reagent with 3% FBS and incubated for 1 hour at 37° C. with (100 nM to 0.006 nM) of test articles. Jurkat PD-1 (SHP2) reporter cells were removed from cell culture, resuspended in cell plating reagent with 3% FBS, and incubated with the Raji cells with test articles for an additional 2 hours at room temperature. Detection reagents were added to each well and luminescence was read using a plate reader. Antagonism produced in reporter cell lines was not observed by antibodies fused to an Fc as shown in FIG. 23.

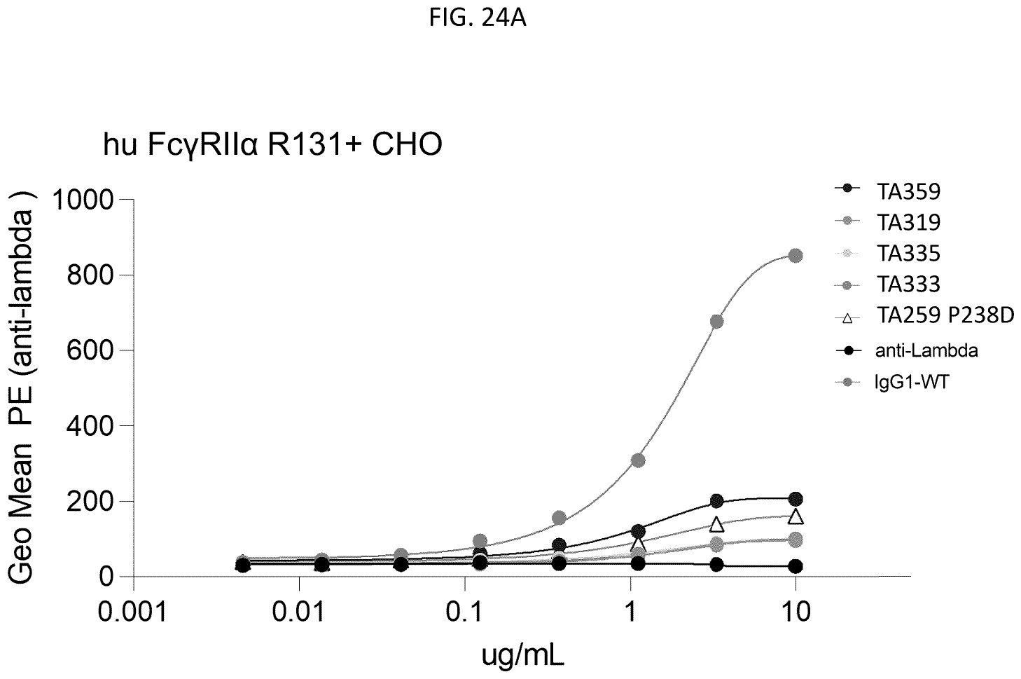

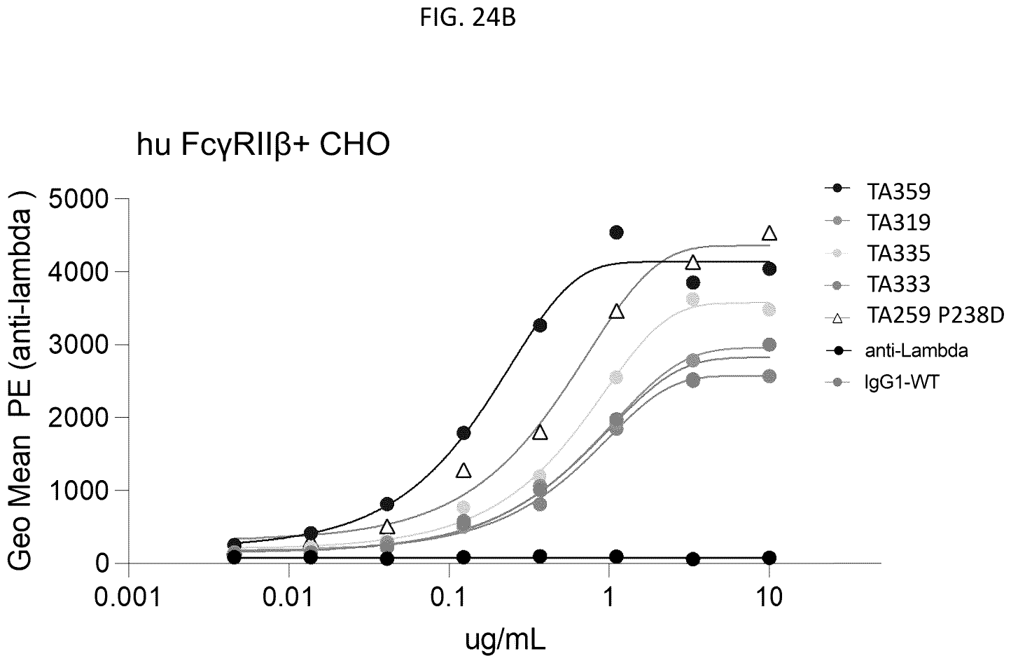

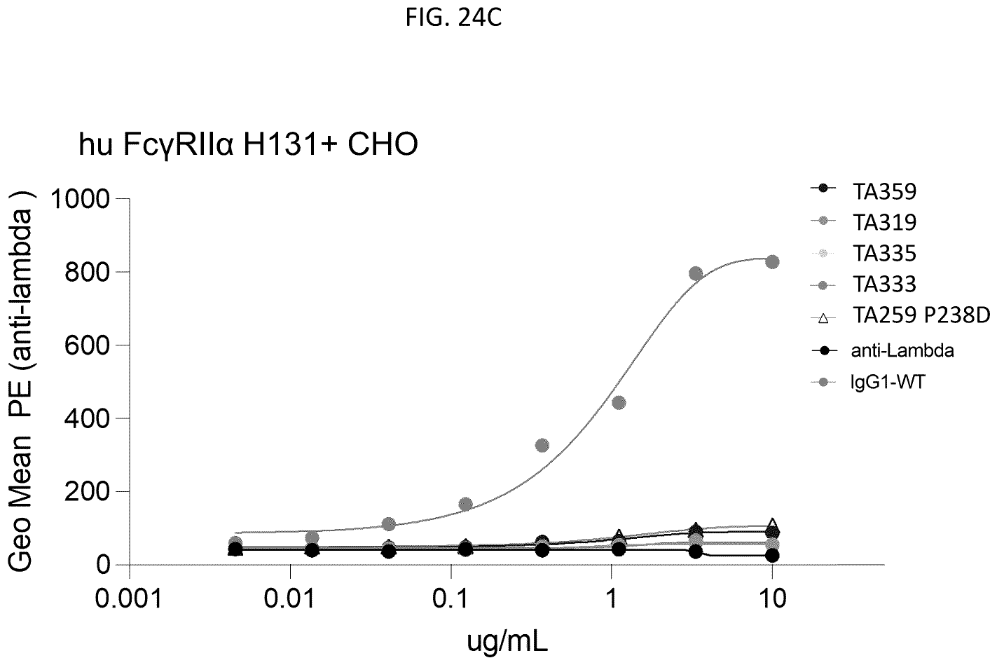

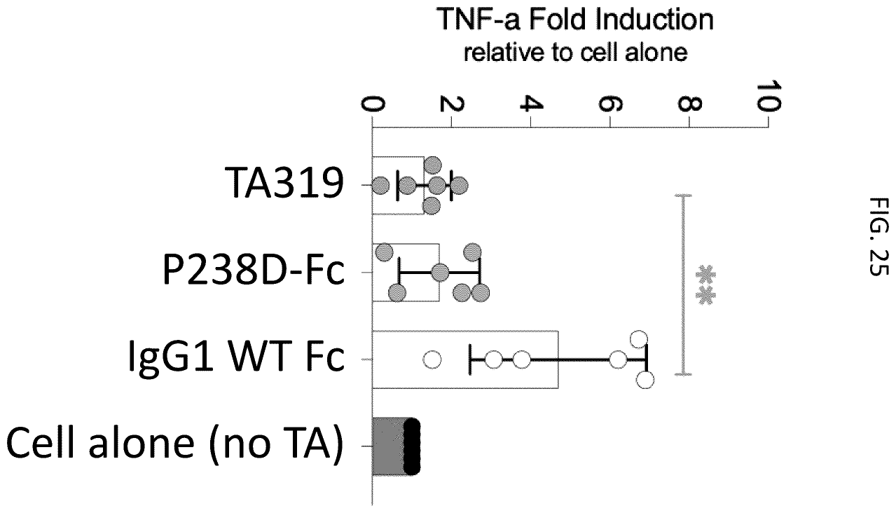

Example 36: Binding of test articles (TA359, TA319, TA335, and TA333) or controls (human TA259 P238D, anti-Lambda, IgG1-WT) to human FcγRIIβ, FcγRIIβ R131 or FcγRIIα H131 was assessed. In brief, human FcγRIIβ, FcγRIIβ R131, or FcγRIIβ H131 were expressed in Chinese hamster ovary (CHO) cells, and binding was assessed following administration of test articles or controls. As shown in FIGS. 24A-24C, test articles showed selectivity for FcγRIIβ as compared to controls.

Example 37: Test articles (TA359, TA322, TA319, TA335, TA333) and a control (benchmark antibody) were evaluated for PD-1 agonism or antagonism using a reporter cell-based assay. Antagonism was not observed in the reporter cells. Agonism was observed in the reporter cells, as shown in Table 27 below.

TABLE 27

| | log EC50 | Emax |
|---|---|---|
| TA359 | −8.3 | 2.2 |
| TA322 | −8.3 | 2.1 |
| TA319 | −9.8 | 1.6 |
| TA335 | −8.2 | 1.8 |
| TA333 | −9.9 | 1.8 |
| Benchmark | −10.1 | 1.9 |
| Antibody | | |

Example 38: Human peripheral blood mononuclear cells (PBMCs) from multiple donors (n=7-10) were cultured and either activated with Staphylococcal Enterotoxin B (SEB) or not activated (control). PBMCs activated with SEB were treated with 100 nM of test articles (TA322, H3L23, TA319, TA335, or TA333) or control molecules (benchmark antibody, or PD-1 agonist fused to wild-type IgG4 Fc) and evaluated for IL-2 expression. Test articles demonstrated a little-to-no induction of IL-2, similarly to cells treated with SEB only.

In another set of experiments, PMBCs from a single donor were activated with SEB and treated with test articles (TA359, TA319, TA335, TA333) or control molecules (benchmark antibody, or PD-1 agonist fused to wild-type IgG4 Fc). Test articles showed lack of IL-2 induction similar to SEB, as compared to controls, which showed IL-2 induction.

Example 39: PMBCs from three donors were activated with SEB and treated with test articles (TA322, H3L23, TA319, TA335, or TA333) or a control molecule (benchmark antibody). PD-1 expression was evaluated and data showed no reduced PD-1 expression, as compared to the control.

In another set of experiments, PMBCs from a single donor were activated with SEB and treated with test articles (TA359, TA319, TA335, TA333) or control molecules (benchmark antibody, or PD-1 agonist fused to wild-type IgG4 Fc). Treatment with test articles resulted in no reduction of PD-1 expression, as compared to the benchmark antibody control.

Example 40: Monocyte derived dendritic cells (DCs) form multiple human donors were exposed to 2 ug of test articles, a benchmark antibody, or untreated. As shown in FIG. 25, treatment with the test articles showed reduced TNF-α induction. Accordingly, reduced TNF-α induction was dependent upon FcγRII clustering on monocyte derived DCs.

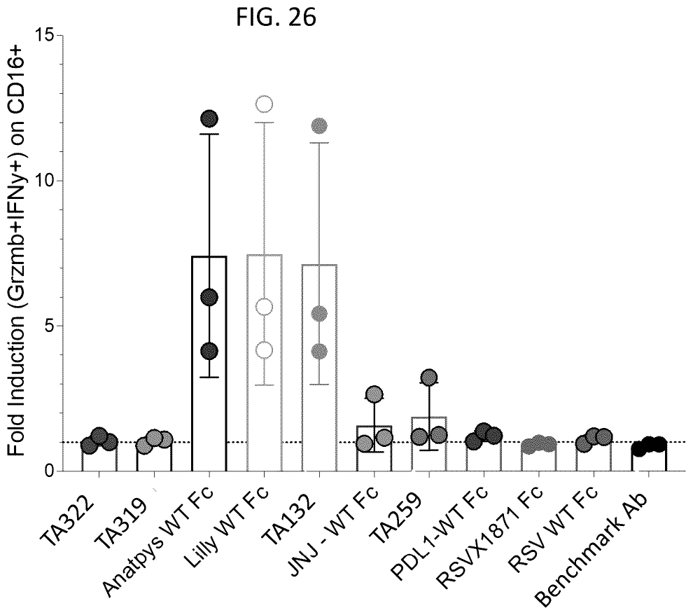

In another experiment, CD16+ PMBCs from multiple human donors were treated with 100 nM of test articles (TA319 or TA322), higher affinity anti-PD-1 antibodies, lower affinity anti-PD-1 antibodies, or control molecules. As shown in FIG. 26, analysis of granzyme b and IFNγ showed a reduction following treatment with test articles, as compared to higher affinity anti-PD-1 antibodies

In another set of experiments, PBMCs from three donors were treated with test articles (TA359, TA319, or TA335) or control molecules (benchmark antibody, or benchmark antibody fused to P238D mutant Fc). Data showed reduced induction of TNF-α following treatment with test articles or the benchmark antibody fused to P238D mutant Fc, as compared to the benchmark antibody which exhibited at least 5-fold TNF-α induction.

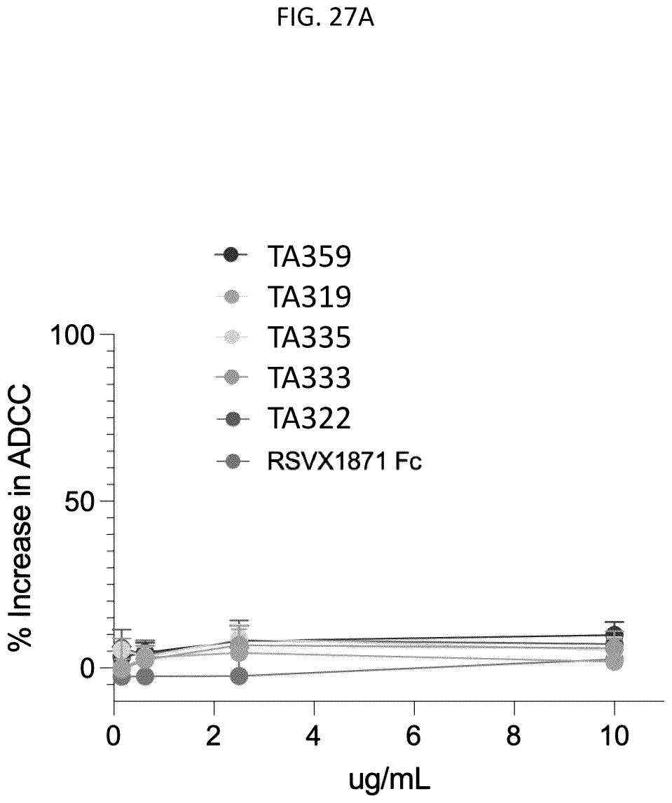

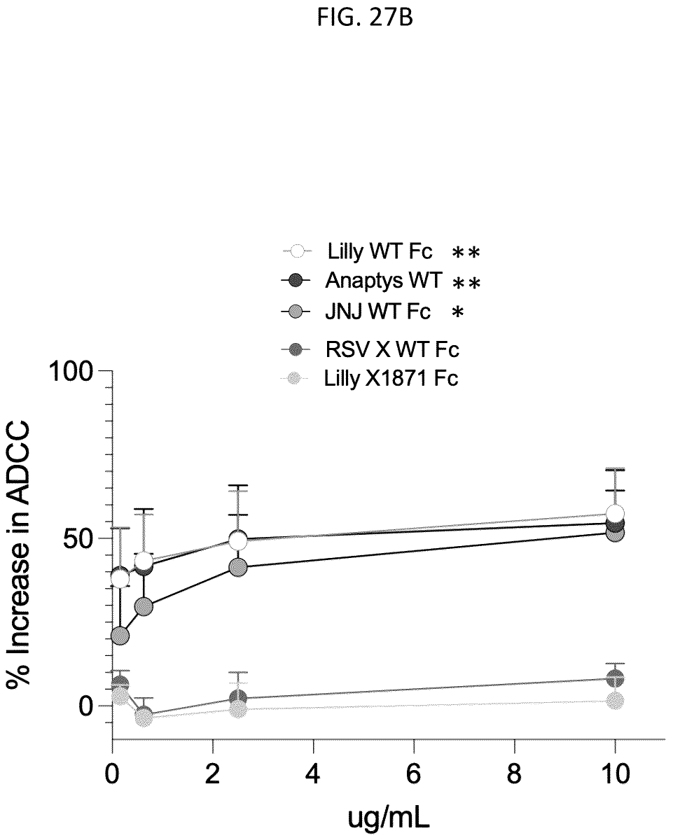

Example 41: Antibody dependent cellular cytotoxicity (ADCC) was evaluated in NK cells from 4 donors. The NK cells were incubated with target Jurkat cells expressing PD-1. ADCC was evaluated following treatment with test articles (TA359, TA319, TA335, or TA333), control molecules, or benchmark molecules As shown in FIG. 27A and FIG. 27B, test articles did not trigger ADCC.

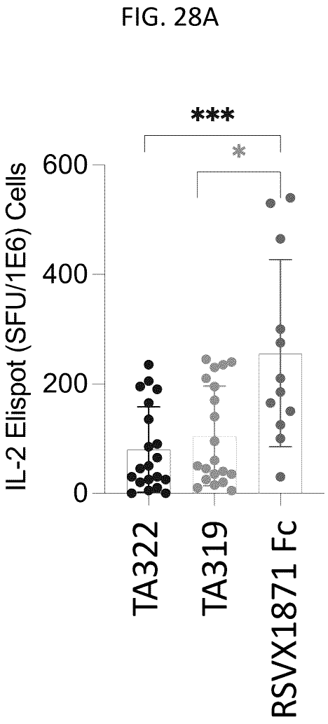

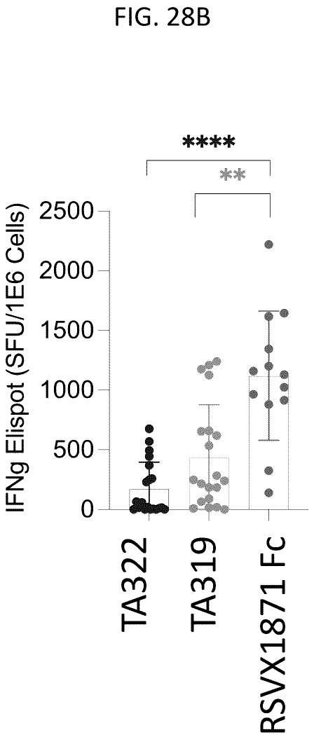

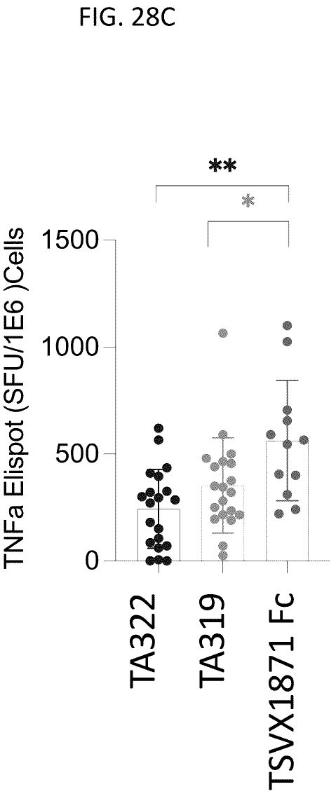

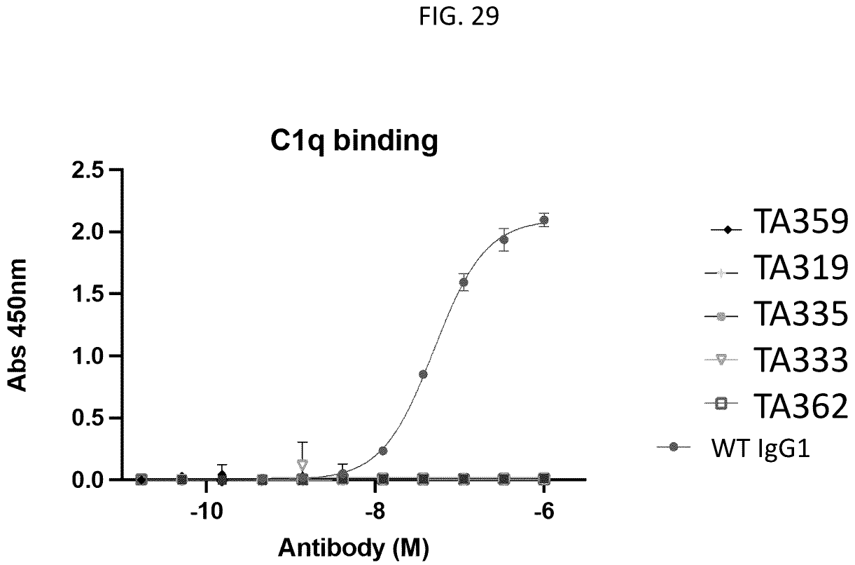

Example 42: Donor cells comprising T-cells collected and depleted from knock-in human FcγR H-2b mice, and T-cells collected and enriched from knock-in human PD-1 H-2b mice, were administered to recipient BDF-1 H-2b,d mice (n=10 per treatment group). Recipient mice were administered test articles (TA322 or TA319) or control molecule twice a week at 1 mpk. Animals treated with test articles showed reduction in IL-2, IFNγ, and TNF-α production, as shown in FIGS. 28A-28C.

Example 43: Analysis of PD-1 binding; FcγRIIβ selectivity and binding to activating receptors FcγRIII and FcγRI of engineered Fc mutants showed diverse affinities for PD-1 binding; FcγRIIβ selectivity and do not bind activating receptors FcγRIII and FcγRI, as shown in Tables 28 and 29 below. Affinity to PD-1 was measured using biolayer inferometry. Test articles were diluted in assay buffer to a final concentration of 5 μg/mL. Recombinant human PD-1 was titrated. Test articles were captured on anti-human IgG Fc biosensors. Association was performed in wells with human PD-1. Dissociation was performed in wells with assay buffer. Kinetic parameters (kon and kdis) and equilibrium dissociation constant (KD) were calculated from a 1:1 Langmuir global Rmax fit model using the data analysis software of the Octet RED96 version 10.0.

TABLE 28
PD-1 variable regions.

| | Human | | | Cyno | | |
|---|---|---|---|---|---|---|
| PD01: | KD | Ka | Kdis | KD | Ka | Kdis |
| FcγRIIβ | (nM) | (1/Ms) | (1/s) | (nM) | (1/Ms) | (1/s) |
| TA359 | 1000 | N/A* | N/A* | 600 | N/A* | N/A* |
| TA319 | 7.3 | 7.2E04 | 5.7E−04 | 73.2 | 1.4E05 | 1.0E−02 |
| TA335 | 151.6 | 6.9E04 | 1.1E−02 | 736.8 | N/A* | N/A* |
| TA333 | 7.5 | 8.6E04 | 6.5E−04 | 97.9 | 1.48E05 | 1.45E−02 |

*Affinity obtained by steady state kinetics

TABLE 29
Engineered Fc mutants

| | KD | KD R2a | KD R2a | R131/ | H131/ | | Affinity |
|---|---|---|---|---|---|---|---|
| PD-1: | R2b | R131 | H131 | 2b | 2b | KD | to |
| FcγRIIβ | (μM) | (μM) | (μM) | (fold) | (fold) | RIII | RI |
| TA322 | 3.5 | 33 | No | 9.7 | Highly | No | >10 fold |
| | | | binding | | selective | binding | lower than |
| | | | | | | | WT |
| TA359 | 3.1 | No | No | Highly | Highly | No | >10 fold |
| | | binding | binding | selective | selective | binding | lower than |
| | | | | | | | WT |
| TA319 | 2.1 | 7.9 | No | 3.2 | Highly | No | >10 fold |
| | | | binding | | selective | binding | lower than |
| | | | | | | | WT |
| TA335 | 2.4 | 6.8 | No | 2.8 | Highly | No | >10 fold |
| | | | binding | | selective | binding | lower than |
| | | | | | | | WT |
| TA333 | 2.3 | 8 | No | 3.5 | Highly | No | >10 fold |
| | | | binding | | selective | binding | lower than |
| | | | | | | | WT |

Example 44: Binding ability of test articles comprising variant Fc molecules with increased affinity for FcγRIIβ to C1q was evaluated via enzyme-linked immunoassay. As shown in FIG. 29, test articles displayed no binding to C1q.

Example 45: Analysis of binding to FcRn of engineered Fc mutants displayed preserved FcRn binding, as shown in Table 30 below.

TABLE 30
Engineered Fc mutants

| | | Acidic | Neutral |
|---|---|---|---|
| | | Dissociation | Dissociation KD |
| Construct | Fc Type | KD (M) | (M) |
| WT Fc fused to anti- | WT | 9.6E−7 | 6.4E−4 |
| PD-1 antibody | | | |
| VFC-88 fused to anti- | VFC-88 | 9.9E−7 | 6.4E−7 |
| PD-1 antibody | | | |

Example 46: Identification of residues for anti-PD-1 antibody binding. Structures of antibody Fab fragments bound to PD-1 were determined by single particle cryo-electron microscopy. Data was collected on a Titan Krios (ThermoFisher Scientific) electron microscope. The data were processed using CryoSPARC software (Structura Biotechnology) and the structural models were built in Coot (free software under GNU General Public License). The complex structures were analyzed using Pymol software (Schrödinger) to determine epitope contact residues, which are defined as amino acid residues in the Fab fragment that are within 4 Å of any atoms in PD-1. Thus, the epitope for TA335 includes residues P39, A40, L41, L42, V43, V44, T45, D48, H107, R143, T145, R147, and R148 of PD-1. The residue numbering is as set forth in SEQ ID NO: 589. The epitope for TA359 includes E61, S62, L128, A129, P130, K131, A132, and Q133 of PD-1. The residue numbering is as set forth in SEQ ID NO: 589. The epitope for TA98 includes E146, R147, R148, A149, E150, V151, P152, and A154 of PD-1 as compared to SEQ ID NO: 589.

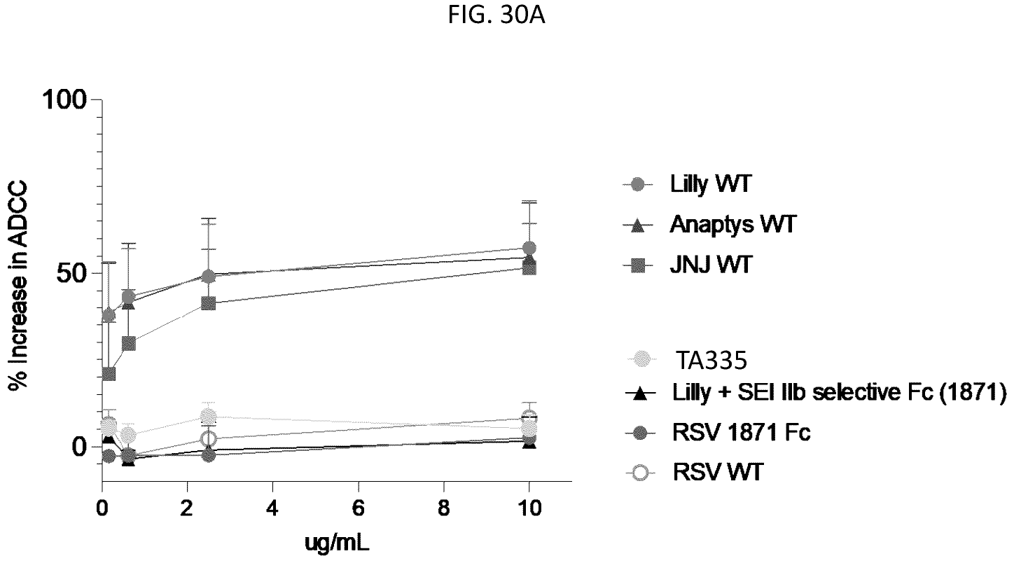

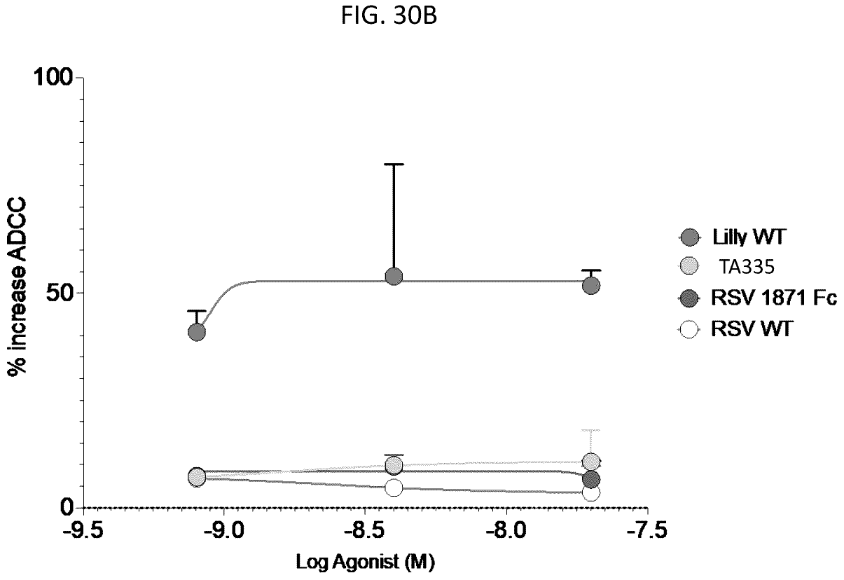

Example 47: Antibody dependent cellular cytotoxicity (ADCC) was evaluated in human or cynomolgus monkey cell cultures by using isolated NK cells or PBMC, respectively. The cells were incubated with target Jurkat cells expressing PD-1. ADCC was evaluated by measuring dead target cells via flow cytometry following treatment with test article (TA335), control molecules, or benchmark molecules. As shown in FIG. 30A and FIG. 30B, TA335 did not trigger ADCC. Without being bound to any theory, the lack of ADCC is believed to be due to its affinity not being to strong, i.e. “low affinity.”

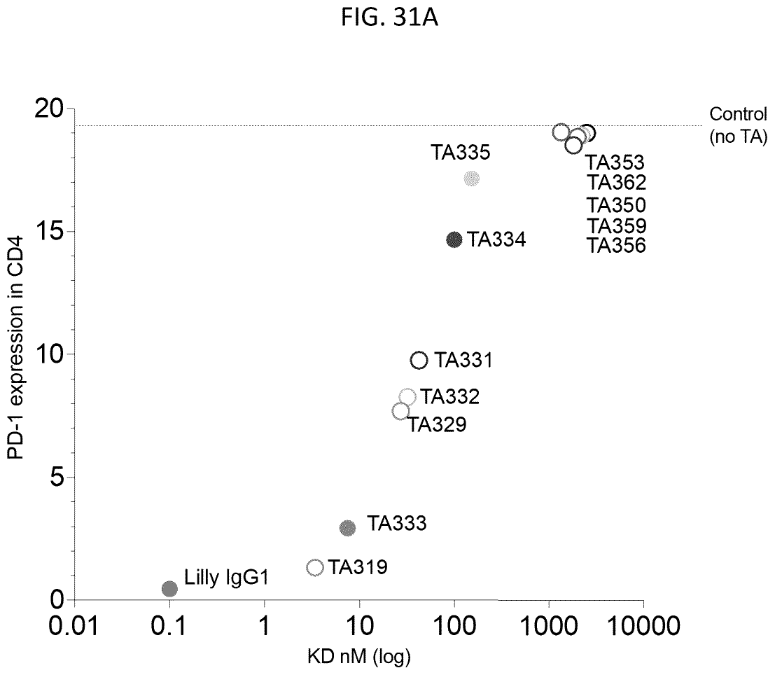

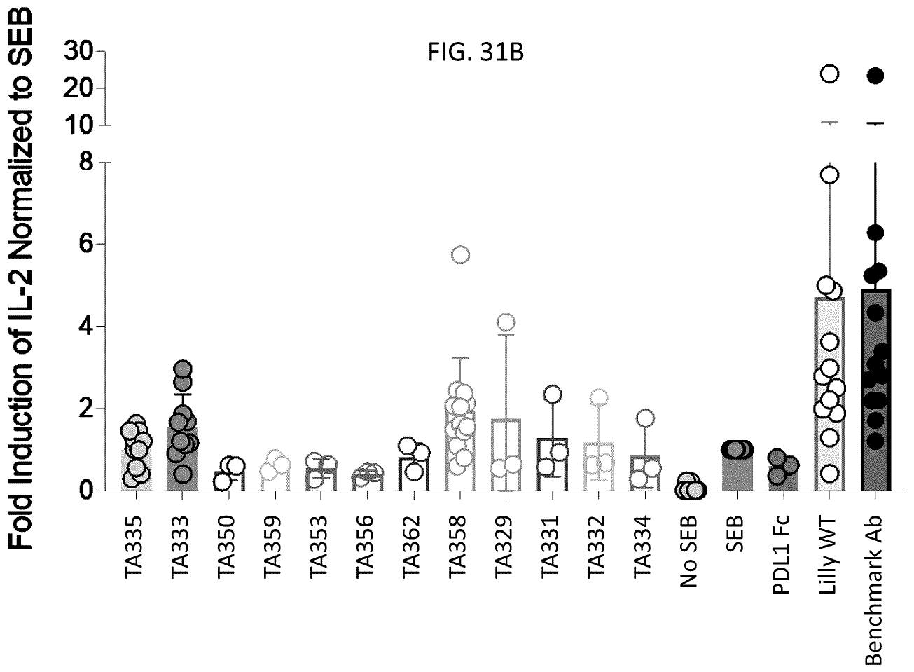

Example 48: PD-1 expression was measured on CD4+ T cells upon PBMC cells activated with SEB in presence or absence of test articles (100 nM). PD-1 binding affinities of anti-PD-1 antibodies is as shown in FIG. 31A. In the same assay, IL-2 induction was measured in multiple donors, and revealed IL-2 production triggered by loss of PD-1 signaling by high-affinity binding anti-PD-1 antibodies, as shown in FIG. 31B

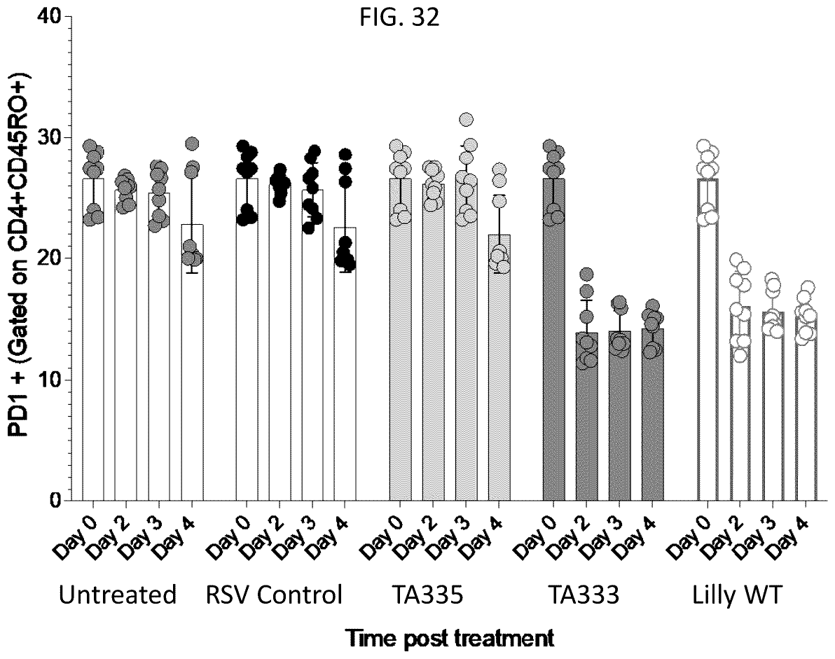

Example 49: PD-1 expression was measured in CD4+CD45RO+ from unstimulated whole blood cell cultures treated with or without test articles. As shown in FIG. 32, high-affinity binding anti-PD-1 antibodies (TA333, Lilly WT) triggered loss of surface PD-1, suggestive of PD-1 internalization, as opposed to low-affinity binding anti-PD-1 antibody (TA335). Thus, the low affinity binding antibodies that bind to an epitope, such as the epitope that TA335 binds to (see, Example 46) have surprising and unexpected properties that make such molecules better therapeutic candidates as compared to PD-1 agonist antibodies that bind with high affinity and/or bind to a different epitope.

Example 50: PD-1 Agonist Antibody Molecule Comprising an FcγRIIβ-selective Fc Polypeptide binds specifically to FcγRIIβ. The binding of TA335 was evaluated for its specificity in binding to FcγRIIβ as compared to two different variants of human FcγRIIα. Affinity to FcγRIIβ/FcγRIIα variants/FcgRIII was measured using surface plasmon resonance. TA335 was diluted in assay buffer to a final concentration of 1 μg/mL. Recombinant human FcγRIIβ was titrated. TA335 was captured on ProAG HC30M chip. Association was performed by injecting human FcγRIIβ/FcγRIIα variants/FcgRIII over immobilized TA335. Dissociation was performed with injections of assay buffer. Kinetic parameters (ka and kd) and equilibrium dissociation constant (KD) were calculated from a 1:1 Langmuir global Rmax fit model using the data analysis software of the Carterra LSA Kinetics Software.

Briefly, cell lines expressing the different receptors were contacted with TA335. Affinity was measured and TA335 was found to be selective for FcγRIIb as compared to the human variants of FcγRIIα tested. The binding to FcRN was not affected. That data is summarized in the Table 35 below.

| | Kd | Kd R2a | Kd R2a | R131/ | H131/ | Kd | |
|---|---|---|---|---|---|---|---|
| PD-1: | R2b | R131 | H131 | 2b | 2b | RIII | FcRn |
| FcgRIIb | (uM) | (uM) | (uM) | (fold) | (fold) | (uM) | (uM) |
| TA335 | ++ | + | No | Selective | Highly | No | +++ |
| | | | Binding | | Selective | Binding | |
| WT | ++ | ++ | ++ | Not | Not | ++ | +++ |
| IgG1 | | | | Selective | Selective | | |

Furthermore, TA335 was analyzed in vivo to determine its effect on Treg. Specifically, TA335 was tested in vivo and was found to increase % of FOXP3+ Tregs. Additionally, Tregs were found to be activated in vivo, which correlates with stronger suppressive functions. The antibody also led to an increase in TIGIT expression on Tregs and/or expanded the TIGIT positive population of T cells, which also correlates with an increase in a more immune suppressive Treg population of cell. These data demonstrate that the molecules provided for herein can be used to treat auto-immune conditions by increasing the number and/or activation of Tregs.

The embodiments and examples provided for herein demonstrate the unexpected and surprising properties of the molecules provided for herein that not only agonize the activity of PD-1, but also specifically bind to FcγRIIb, which have superior properties to other antibodies that have a negative effect on PD-1 recycling as well as on ADCC activity.

Example 51: Purified anti-FcγRIIβ antibody test articles were generated for binding and functional assays. Said test articles were expressed in Expi293F cells. Each antibody test article was purified by passing it through a 5 mL PrismA column. Target antibodies were eluted with 0.1M Glycine at pH 2.8, and neutralized immediately using 5% 1M Tris HCl at pH 8.0. The eluted samples were loaded to analytical SEC to check for monodispersity of Protein of Interest (POI). All test articles were purified with the majority population present as monomers, as shown in Table 31 below.

TABLE 31

| Test Articles | monomeric POI (%) | Yield (mg/L) |
|---|---|---|
| ARB43 | 97.3 | 5.8 |
| ARB52 | 96.5 | 3.9 |
| ARB61 | 97 | 9.4 |
| ARB70 | 97.8 | 5.1 |
| ARB40 | 95.5 | 4.3 |
| ARB49 | 96.2 | 5.3 |
| ARB58 | 96.3 | 3.6 |
| ARB67 | 97.1 | 3.6 |

Example 52: The binding of anti-FcγRIIβ antibodies to human FcγRIIβ was evaluated. Antibodies were captured on ProA/G HC30M chip at 5 ug/mL, 1 ug/mL, and 0.2 ug/mL for 15 minutes. Six buffer injections were made over the chip followed by injections of increasing concentrations of recombinant human FcγRIIβ (4 nm-5 uM). The association was measured during each injection for 5 minutes and the dissociation was measured for 10 minutes post injection with buffer flow (1×HBSTE+ BSA). Results are shown in Table 32 below.

TABLE 32

| | Average Kinetic | Average Steady State |
|---|---|---|
| Name | KD (M) | KD (M) |
| ARB40 | 1.2E−07 | 1.2E−07 |
| ARB49 | 3.73E−08 | 4.0E−08 |
| ARB58 | 2.07E−07 | 1.8E−07 |
| ARB67 | 3.8E−06 | 3.5E−06 |
| Benchmark Ab | 2.43E−08 | N/A |
| 2B6 | 4.07E−10 | N/A |
| V12 Fc | 2.13E−08 | 2.1E−08 |
| 6G08 | 1.73E−06 | 1.7E−06 |

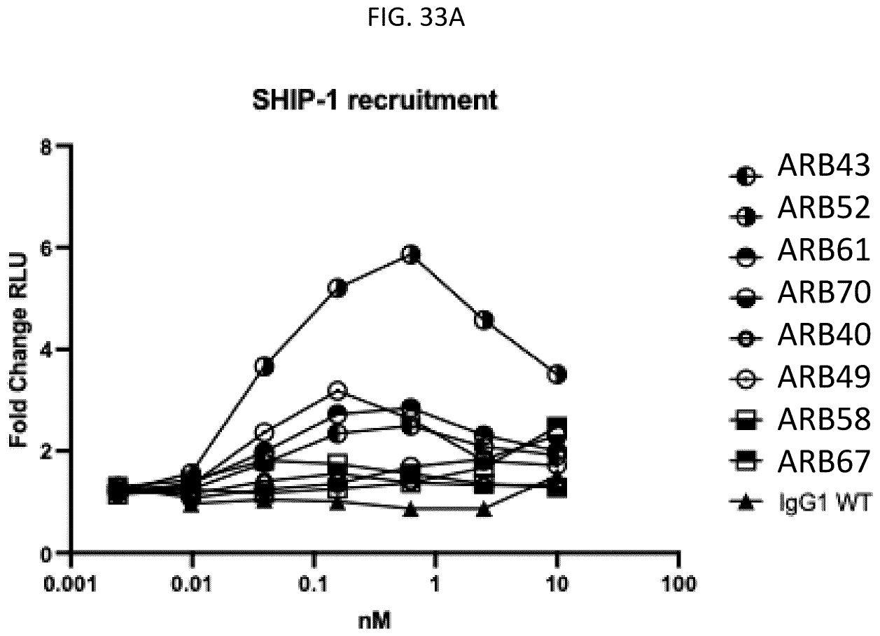

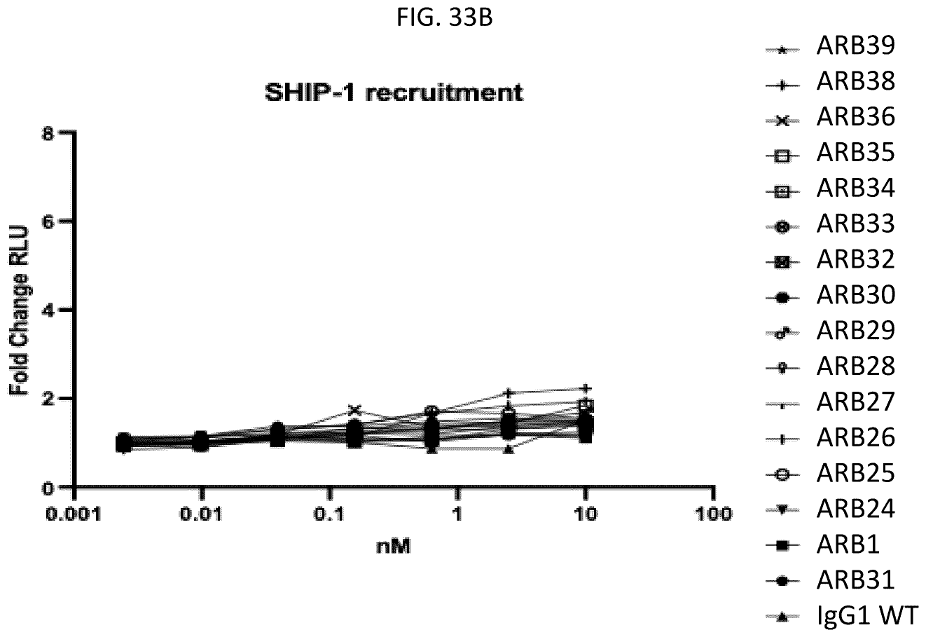

Example 53:293 T cells engineered to express only FcγRIIβ (Promega-NanoBiT® SHIP-1) were harvested from and suspended in Opti-MEM+5% low IgG FBS and plated into a 96-well plate at the density of 5×10\hat{ }4 cells/well. Nano-Glo® Live Cell Reagent was added, followed by serial dilutions of test article. Luminescence was recorded after 30 minute incubation at 37° C., 5% CO2. Analysis of the data was performed with GraphPad Prism® software, reporter as fold change RLU compared to cells alone. Results show several Fab format molecules capable of mediating the recruitment of SHIP-1 to FcγRIIβ in absence of any activating signal derived from FcγRIIα (FIG. 33A), as opposed to molecules in scFv format (FIG. 33B).

Thus, the antibodies that are specific for FcγRIIβ can be used to negatively regulate an immune response, and, thus can be used to treat various auto-immune diseases, such as those provided for herein.

The disclosures of each and every patent, patent application, accession number, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

1-60. (canceled)

61. An anti-PD-1 antibody, or an antigen-binding fragment thereof, that binds to an epitope on PD-1 that comprises residues P39, A40, L41, L42, V43, V44, T45, D48, E61, S62, H107, L128, A129, P130, K131, A132, Q133, R143, T145, E146, R147, R148, A149, E150, V151, P152, A154, or any combination thereof.

62. The anti-PD-1 antibody, or the antigen-binding fragment thereof, of claim 61, wherein the anti-PD-1 antibody, or an antigen-binding fragment thereof, binds to an epitope of PD-1 with a low binding affinity and the antibody is a PD-1 agonist.

63. The anti-PD-1 antibody, or an antigen-binding fragment thereof, of claim 61, wherein the antibody comprises:
a variable heavy chain comprising a HCDR1 having an amino acid sequence of any one SEQ ID NOs: 553, 547, 550, or 556, a HCDR2 having an amino acid sequence of any one SEQ ID NOs: 554, 548, 551, or 557, and a HCDR3 having an amino acid sequence of any one SEQ ID NOs: 555, 549, 552, or 558; and
a variable light chain comprising a LCDR1 having an amino acid sequence of any one SEQ ID NOs: 574, 559, 562, 565, 568, 569, 570, 571, 572, or 573, a LCDR2 having an amino acid sequence of any one SEQ ID NOs: 563, 560, or 566, and a LCDR3 having an amino acid sequence of any one SEQ ID NOs: 564, 561, or 567.

64. The anti-PD-1 antibody, or the antigen-binding fragment thereof, of claim 61, wherein the antibody, or the antigen-binding fragment thereof, comprises:
a variable heavy chain domain (VH) comprising an amino acid sequence that is at least 90-99% identical to the amino acid sequence of SEQ ID NO: 530, provided that the VH comprises a HCDR1 of SEQ ID NO: 553, a HCDR2 of SEQ ID NO: 554, and a HCDR3 of SEQ ID NO: 555; and
a variable light chain domain (VL) comprising an amino acid sequence that is at least 90-99% identical to the amino acid sequence of SEQ ID NO: 537, provided that the VL comprises a LCDR1 of SEQ ID NO: 574, a LCDR2 of SEQ ID NO: 563, and a LCDR3 of SEQ ID NO: 564.

65. The antibody of claim 64, wherein the antibody comprises a VH comprising the amino acid sequence of SEQ ID NO: 530 and a VL comprising the amino acid sequence of SEQ ID NO: 537.

66. The antibody of claim 65, wherein the antibody comprises a heavy chain (HC) comprising an amino acid sequence that is at least 90-99% identical to the amino acid sequence of the amino acid sequence of SEQ ID NO: 691 and a light chain (LC) comprising an amino acid sequence that is at least 90-99% identical to the amino acid sequence of SEQ ID NO: 692.

67. The antibody, or antigen fragment thereof, of claim 66, wherein the HC comprises the amino acid sequence of SEQ ID NO: 691 and the LC comprises the amino acid sequence of SEQ ID NO: 692.

68. The anti-PD-1 antibody, or the antigen-binding fragment thereof, of claim 61, wherein the anti-PD-1 antibody, or the antigen-binding fragment thereof, comprising a VH is conjugated to an Fc polypeptide.

69. The anti-PD-1 antibody, or the antigen-binding fragment thereof, of claim 68, wherein the Fc polypeptide comprises an amino acid sequence having at least 90-99% sequence identity to any one of SEQ ID NOs: 543, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 544, 545, 546, 683, 684, 685, 686, 687, 688, 689, and 690, and
wherein the Fc polypeptide selectively binds to FcγRIIβ.

70. The anti-PD-1 antibody, or the antigen-binding fragment thereof, of claim 69, wherein the Fc polypeptide comprises the amino acid sequence of any one of SEQ ID NOs: 543, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 544, 545, 546, 683, 684, 685, 686, 687, 688, 689, and 690.

71. A pharmaceutical composition comprising the antibody, or the antigen-binding fragment thereof, of claim 61 and a pharmaceutically acceptable excipient.

72. A nucleic acid molecule encoding the antibody, or the antigen-binding fragment thereof, of claim 61.

73. A cell comprising the nucleic acid molecule of claim 72.

74. A method of:
treating an autoimmune disorder in a subject, the method comprising administering a pharmaceutical composition comprising the antibody, or the antigen-binding fragment thereof, of claim 61 and a pharmaceutically acceptable excipient;
increasing FoxP3+ Tregs in a subject, the method comprising administering a pharmaceutical composition comprising the antibody, or the antigen-binding fragment thereof, of claim 61 and a pharmaceutically acceptable excipient;
increasing TIGIT expression in FoxP3+ Tregs in a subject or a method of expanding TIGIT+, FOXP3+ Tregs in a subject, the method comprising administering a pharmaceutical composition comprising the antibody, or the antigen-binding fragment thereof, of claim 61 and a pharmaceutically acceptable excipient; or
modulating a T cell expressing PD-1 and/or B-Cell expressing FcγRIIβ, the method comprising administering a pharmaceutical composition comprising the antibody, or the antigen-binding fragment thereof, of claim 61 and a pharmaceutically acceptable excipient.

75. An anti-FcγRIIβ antibody, or an antigen-binding fragment thereof, comprising a polypeptide comprising:
a variable heavy chain comprising a HCDR1 having an amino acid sequence of any one SEQ ID NOs: 606, 575, 578, 590, 595, or 601, a HCDR2 having an amino acid sequence of any one SEQ ID NOs: 607, 576, 579, 591, 596, or 602, and a HCDR3 having an amino acid sequence of any one SEQ ID NOs: 608, 577, 580, 592, 597, or 603; and
a variable light chain comprising a LCDR1 having an amino acid sequence of any one SEQ ID NOs: 609, 581, 584, 593, 598, or 604, a LCDR2 having an amino acid sequence of any one SEQ ID NOs: 610, 582, 585, 594, or 599, and a LCDR3 having an amino acid sequence of any one SEQ ID NOs: 586, 583, 600, or 605, and
wherein the anti-FcγRIIβ antibody, or the antigen-binding fragment thereof, binds selectively to FcγRIIβ.

76. The anti-FcγRIIβ antibody, or the antigen-binding fragment thereof, of claim 75, wherein the polypeptide comprises:
i.
a variable light chain having an amino acid sequence having at least 90-99% sequence identity to SEQ ID NO: 44, and a variable heavy chain having an amino acid sequence having at least 90-99% sequence identity to SEQ ID NO: 84;
a variable light chain having an amino acid sequence having at least 90-99% sequence identity to SEQ ID NO: 18, and a variable heavy chain having an amino acid sequence having at least 90-99% sequence identity to SEQ ID NO: 54;
a variable light chain having an amino acid sequence having at least 90-99% sequence identity to SEQ ID NO: 19, and a variable heavy chain having an amino acid sequence having at least 90-99% sequence identity to SEQ ID NO: 55;
a variable light chain having an amino acid sequence having at least 90-99% sequence identity to SEQ ID NO: 20, and a variable heavy chain having an amino acid sequence having at least 90-99% sequence identity to SEQ ID NO: 56;
a variable light chain having an amino acid sequence having at least 90-99% sequence identity to SEQ ID NO: 21, and a variable heavy chain having an amino acid sequence having at least 90-99% sequence identity to SEQ ID NO: 57;
a variable light chain having an amino acid sequence having at least 90-99% sequence identity to SEQ ID NO: 22, and a variable heavy chain having an amino acid sequence having at least 90-99% sequence identity to SEQ ID NO: 58;
a variable light chain having an amino acid sequence having at least 90-99% sequence identity to SEQ ID NO: 23, and a variable heavy chain having an amino acid sequence having at least 90-99% sequence identity to SEQ ID NO: 59;
a variable light chain having an amino acid sequence having at least 90-99% sequence identity to SEQ ID NO: 24, and a variable heavy chain having an amino acid sequence having at least 90-99% sequence identity to SEQ ID NO: 60;
a variable light chain having an amino acid sequence having at least 90-99% sequence identity to SEQ ID NO: 25, and a variable heavy chain having an amino acid sequence having at least 90-99% sequence identity to SEQ ID NO: 61;
a variable light chain having an amino acid sequence having at least 90-99% sequence identity to SEQ ID NO: 26, and a variable heavy chain having an amino acid sequence having at least 90-99% sequence identity to SEQ ID NO: 62;
a variable light chain having an amino acid sequence having at least 90-99% sequence identity to SEQ ID NO: 27, and a variable heavy chain having an amino acid sequence having at least 90-99% sequence identity to SEQ ID NO: 63;
a variable light chain having an amino acid sequence having at least 90-99% sequence identity to SEQ ID NO: 28, and a variable heavy chain having an amino acid sequence having at least 90-99% sequence identity to SEQ ID NO: 64;
a variable light chain having an amino acid sequence having at least 90-99% sequence identity to SEQ ID NO: 29, and a variable heavy chain having an amino acid sequence having at least 90-99% sequence identity to SEQ ID NO: 65;
a variable light chain having an amino acid sequence having at least 90-99% sequence identity to SEQ ID NO: 30, and a variable heavy chain having an amino acid sequence having at least 90-99% sequence identity to SEQ ID NO: 66;
a variable light chain having an amino acid sequence having at least 90-99% sequence identity to SEQ ID NO: 31, and a variable heavy chain having an amino acid sequence having at least 90-99% sequence identity to SEQ ID NO: 67;
a variable light chain having an amino acid sequence having at least 90-99% sequence identity to SEQ ID NO: 32, and a variable heavy chain having an amino acid sequence having at least 90-99% sequence identity to SEQ ID NO: 68;
a variable light chain having an amino acid sequence having at least 90-99% sequence identity to SEQ ID NO: 33, and a variable heavy chain having an amino acid sequence having at least 90-99% sequence identity to SEQ ID NO: 69;
a variable light chain having an amino acid sequence having at least 90-99% sequence identity to SEQ ID NO: 34, and a variable heavy chain having an amino acid sequence having at least 90-99% sequence identity to SEQ ID NO: 70;
a variable light chain having an amino acid sequence having at least 90-99% sequence identity to SEQ ID NO: 35, and a variable heavy chain having an amino acid sequence having at least 90-99% sequence identity to SEQ ID NO: 71;
a variable light chain having an amino acid sequence having at least 90-99% sequence identity to SEQ ID NO: 36, and a variable heavy chain having an amino acid sequence having at least 90-99% sequence identity to SEQ ID NO: 72;
a variable light chain having an amino acid sequence having at least 90-99% sequence identity to SEQ ID NO: 37, and a variable heavy chain having an amino acid sequence having at least 90-99% sequence identity to SEQ ID NO: 73;
a variable light chain having an amino acid sequence having at least 90-99% sequence identity to SEQ ID NO: 38, and a variable heavy chain having an amino acid sequence having at least 90-99% sequence identity to SEQ ID NO: 74;
a variable light chain having an amino acid sequence having at least 90-99% sequence identity to SEQ ID NO: 39, and a variable heavy chain having an amino acid sequence having at least 90-99% sequence identity to SEQ ID NO: 75;
a variable light chain having an amino acid sequence having at least 90-99% sequence identity to SEQ ID NO: 40, and a variable heavy chain having an amino acid sequence having at least 90-99% sequence identity to SEQ ID NO: 76;
a variable light chain having an amino acid sequence having at least 90-99% sequence identity to SEQ ID NO: 41, and a variable heavy chain having an amino acid sequence having at least 90-99% sequence identity to SEQ ID NO: 77;
a variable light chain having an amino acid sequence having at least 90-99% sequence identity to SEQ ID NO: 42, and a variable heavy chain having an amino acid sequence having at least 90-99% sequence identity to SEQ ID NO: 78;
a variable light chain having an amino acid sequence having at least 90-99% sequence identity to SEQ ID NO: 43, and a variable heavy chain having an amino acid sequence having at least 90-99% sequence identity to SEQ ID NO: 79;
a variable light chain having an amino acid sequence having at least 90-99% sequence identity to SEQ ID NO: 44, and a variable heavy chain having an amino acid sequence having at least 90-99% sequence identity to SEQ ID NO: 80;
a variable light chain having an amino acid sequence having at least 90-99% sequence identity to SEQ ID NO: 45, and a variable heavy chain having an amino acid sequence having at least 90-99% sequence identity to SEQ ID NO: 81;
a variable light chain having an amino acid sequence having at least 90-99% sequence identity to SEQ ID NO: 46, and a variable heavy chain having an amino acid sequence having at least 90-99% sequence identity to SEQ ID NO: 80;
a variable light chain having an amino acid sequence having at least 90-99% sequence identity to SEQ ID NO: 47, and a variable heavy chain having an amino acid sequence having at least 90-99% sequence identity to SEQ ID NO: 82;
a variable light chain having an amino acid sequence having at least 90-99% sequence identity to SEQ ID NO: 48, and a variable heavy chain having an amino acid sequence having at least 90-99% sequence identity to SEQ ID NO: 83;
a variable light chain having an amino acid sequence having at least 90-99% sequence identity to SEQ ID NO: 49, and a variable heavy chain having an amino acid sequence having at least 90-99% sequence identity to SEQ ID NO: 85;
a variable light chain having an amino acid sequence having at least 90-99% sequence identity to SEQ ID NO: 44, and a variable heavy chain having an amino acid sequence having at least 90-99% sequence identity to SEQ ID NO: 86;
a variable light chain having an amino acid sequence having at least 90-99% sequence identity to SEQ ID NO: 50, and a variable heavy chain having an amino acid sequence having at least 90-99% sequence identity to SEQ ID NO: 87;
a variable light chain having an amino acid sequence having at least 90-99% sequence identity to SEQ ID NO: 51, and a variable heavy chain having an amino acid sequence having at least 90-99% sequence identity to SEQ ID NO: 88;
a variable light chain having an amino acid sequence having at least 90-99% sequence identity to SEQ ID NO: 52, and a variable heavy chain having an amino acid sequence having at least 90-99% sequence identity to SEQ ID NO: 88;
a variable light chain having an amino acid sequence having at least 90-99% sequence identity to SEQ ID NO: 53, and a variable heavy chain having an amino acid sequence having at least 90-99% sequence identity to SEQ ID NO: 89; or
a variable light chain having an amino acid sequence having at least 90-99% sequence identity to SEQ ID NO: 44, and a variable heavy chain having an amino acid sequence having at least 90-99% sequence identity to SEQ ID NO: 90,
wherein the referenced variable light chain and the variable heavy chain comprise the CDRs as set forth herein for the referenced variable light chain and the variable heavy chain; or
ii.
a variable light chain having an amino acid sequence of SEQ ID NO: 44, and a variable heavy chain having an amino acid sequence of SEQ ID NO: 84;
a variable light chain having an amino acid sequence of SEQ ID NO: 18, and a variable heavy chain having an amino acid sequence of SEQ ID NO: 54;
a variable light chain having an amino acid sequence of SEQ ID NO: 19, and a variable heavy chain having an amino acid sequence of SEQ ID NO: 55;
a variable light chain having an amino acid sequence of SEQ ID NO: 20, and a variable heavy chain having an amino acid sequence of SEQ ID NO: 56;
a variable light chain having an amino acid sequence of SEQ ID NO: 21, and a variable heavy chain having an amino acid sequence of SEQ ID NO: 57;
a variable light chain having an amino acid sequence of SEQ ID NO: 22, and a variable heavy chain having an amino acid sequence of SEQ ID NO: 58;
a variable light chain having an amino acid sequence of SEQ ID NO: 23, and a variable heavy chain having an amino acid sequence of SEQ ID NO: 59;
a variable light chain having an amino acid sequence of SEQ ID NO: 24, and a variable heavy chain having an amino acid sequence of SEQ ID NO: 60;
a variable light chain having an amino acid sequence of SEQ ID NO: 25, and a variable heavy chain having an amino acid sequence of SEQ ID NO: 61;
a variable light chain having an amino acid sequence of SEQ ID NO: 26, and a variable heavy chain having an amino acid sequence of SEQ ID NO: 62;
a variable light chain having an amino acid sequence of SEQ ID NO: 27, and a variable heavy chain having an amino acid sequence of SEQ ID NO: 63;
a variable light chain having an amino acid sequence of SEQ ID NO: 28, and a variable heavy chain having an amino acid sequence of SEQ ID NO: 64;
a variable light chain having an amino acid sequence of SEQ ID NO: 29, and a variable heavy chain having an amino acid sequence of SEQ ID NO: 65;
a variable light chain having an amino acid sequence of SEQ ID NO: 30, and a variable heavy chain having an amino acid sequence of SEQ ID NO: 66;
a variable light chain having an amino acid sequence of SEQ ID NO: 31, and a variable heavy chain having an amino acid sequence of SEQ ID NO: 67;
a variable light chain having an amino acid sequence of SEQ ID NO: 32, and a variable heavy chain having an amino acid sequence of SEQ ID NO: 68;
a variable light chain having an amino acid sequence of SEQ ID NO: 33, and a variable heavy chain having an amino acid sequence of SEQ ID NO: 69;
a variable light chain having an amino acid sequence of SEQ ID NO: 34, and a variable heavy chain having an amino acid sequence of SEQ ID NO: 70;
a variable light chain having an amino acid sequence of SEQ ID NO: 35, and a variable heavy chain having an amino acid sequence of SEQ ID NO: 71;
a variable light chain having an amino acid sequence of SEQ ID NO: 36, and a variable heavy chain having an amino acid sequence of SEQ ID NO: 72;
a variable light chain having an amino acid sequence of SEQ ID NO: 37, and a variable heavy chain having an amino acid sequence of SEQ ID NO: 73;
a variable light chain having an amino acid sequence of SEQ ID NO: 38, and a variable heavy chain having an amino acid sequence of SEQ ID NO: 74;
a variable light chain having an amino acid sequence of SEQ ID NO: 39, and a variable heavy chain having an amino acid sequence of SEQ ID NO: 75;
a variable light chain having an amino acid sequence of SEQ ID NO: 40, and a variable heavy chain having an amino acid sequence of SEQ ID NO: 76;
a variable light chain having an amino acid sequence of SEQ ID NO: 41, and a variable heavy chain having an amino acid sequence of SEQ ID NO: 77;
a variable light chain having an amino acid sequence of SEQ ID NO: 42, and a variable heavy chain having an amino acid sequence of SEQ ID NO: 78;
a variable light chain having an amino acid sequence of SEQ ID NO: 43, and a variable heavy chain having an amino acid sequence of SEQ ID NO: 79;
a variable light chain having an amino acid sequence of SEQ ID NO: 44, and a variable heavy chain having an amino acid sequence of SEQ ID NO: 80;
a variable light chain having an amino acid sequence of SEQ ID NO: 45, and a variable heavy chain having an amino acid sequence of SEQ ID NO: 81;
a variable light chain having an amino acid sequence of SEQ ID NO: 46, and a variable heavy chain having an amino acid sequence of SEQ ID NO: 80;
a variable light chain having an amino acid sequence of SEQ ID NO: 47, and a variable heavy chain having an amino acid sequence of SEQ ID NO: 82;
a variable light chain having an amino acid sequence of SEQ ID NO: 48, and a variable heavy chain having an amino acid sequence of SEQ ID NO: 83;
a variable light chain having an amino acid sequence of SEQ ID NO: 49, and a variable heavy chain having an amino acid sequence of SEQ ID NO: 85;
a variable light chain having an amino acid sequence of SEQ ID NO: 44, and a variable heavy chain having an amino acid sequence of SEQ ID NO: 86;
a variable light chain having an amino acid sequence of SEQ ID NO: 50, and a variable heavy chain having an amino acid sequence of SEQ ID NO: 87;
a variable light chain having an amino acid sequence of SEQ ID NO: 51, and a variable heavy chain having an amino acid sequence of SEQ ID NO: 88;
a variable light chain having an amino acid sequence of SEQ ID NO: 52, and a variable heavy chain having an amino acid sequence of SEQ ID NO: 88;
a variable light chain having an amino acid sequence of SEQ ID NO: 53, and a variable heavy chain having an amino acid sequence of SEQ ID NO: 89; or
a variable light chain having an amino acid sequence of SEQ ID NO: 44, and a variable heavy chain having an amino acid sequence of SEQ ID NO: 90.

77. The anti-FcγRIIβ antibody, or the antigen-binding fragment thereof, of claim 75, wherein the anti-FcγRIIβ antibody, or the antigen-binding fragment thereof, is conjugated to an Fc polypeptide.

78. The anti-FcγRIIβ antibody, or the antigen-binding fragment thereof, of claim 77, wherein the Fc polypeptide is:
effectorless, which may have mutations such as LALA (L234A, L235A), or AAA/LALAGA (L234A, L235A, G237A);
selectively binds to FcγRIIβ; or
comprises the amino acid sequence of any one of SEQ ID NOs: 543, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 544, 545, 546, 683, 684, 685, 686, 687, 688, 689, and 690.

79. A pharmaceutical composition comprising the antibody, or the antigen-binding fragment thereof, of claim 75 and a pharmaceutically acceptable excipient.

80. A method of treating an autoimmune disorder in a subject, the method comprising administering a pharmaceutical composition comprising the antibody, or the antigen-binding fragment thereof, of claim 75 and a pharmaceutically acceptable excipient.


SEQUENCE LISTING (692 sequences)

SEQ ID NO: 1 - protein, 5 aa, synthetic construct
GGGGS

SEQ ID NO: 2 - protein, 10 aa, synthetic construct
GGGGSGGGGS

SEQ ID NO: 3 - protein, 15 aa, synthetic construct
GGGGSGGGGSGGGGS

SEQ ID NO: 4 - protein, 20 aa, synthetic construct
GGGGSGGGGSGGGGSGGGGS

SEQ ID NO: 5 - protein, 8 aa, synthetic construct
GGGGGGGG

SEQ ID NO: 6 - protein, 6 aa, synthetic construct
GGGGGG

SEQ ID NO: 7 - protein, 15 aa, synthetic construct
EAAAKEAAAKEAAAK

SEQ ID NO: 8 - protein, 4 aa, synthetic construct
EAAK

SEQ ID NO: 9 - protein, 8 aa, synthetic construct
EAAKEAAK

SEQ ID NO: 10 - protein, 12 aa, synthetic construct
EAAKEAAKEAAK

SEQ ID NO: 11 - protein, 46 aa, synthetic construct
AEAAAKEAAAKEAAAKEAAAKALEAEAAAKEAAAKEAAAKEAAAKA

SEQ ID NO: 12 - protein, 12 aa, synthetic construct
AEAAAKEAAAKA

SEQ ID NO: 13 - protein, 6 aa, synthetic construct
GGGGSG

SEQ ID NO: 14-17 - skipped (4 entries)

SEQ ID NO: 18 - protein, 112 aa, synthetic construct
DIVMTQSPLSLPVTPGEPASISCRSSQSLVHGSGYTFLHWYLQKPGQSPQLLIYDAVTRATGVPDRFSGSGSGTDFTLKISRVEAEDVGVYYCMQTTEFPYTFGGGTKVEIK

SEQ ID NO: 19 - protein, 112 aa, synthetic construct
DIVMTQSPLSLPVTPGEPASISCRSSQSLLHSTGYNFLHWYLQKPGQSPQLLIYDASKRAPGVPDRFSGSGSGTDFTLKISRVEAEDVGVYYCMQTVELPFTFGGGTKVEIK

SEQ ID NO: 20 - protein, 107 aa, synthetic construct
EIVMTQSPATLSLSPGERATLSCRASQSVDRHLAWYQQKPGQAPRLLIYDVSNRAPGIPARFSGSGSGTDFTLTISSLEPEDFAVYYCQQYGWPSITFGGGTKVEIK

SEQ ID NO: 21 - protein, 112 aa, synthetic construct
DIVMTQSPLSLPVTPGEPASISCRSSQSLLSSYGYHNLHWYLQKPGQSPQLLIYDAYVRATGVPDRFSGSGSGTDFTLKISRVEAEDVGVYYCMQSKELPYTFGGGTKVEIK

SEQ ID NO: 22 - protein, 108 aa, synthetic construct
DIQMTQSPSSVSASVGDRVTITCRASQDIGSNLAWYQQKPGKAPKLLIYSGSTLQSGVPSRFSGSGSGTDFTLTISSLQPEDFANYYCQQTSSSPPYTFGGGTKVEIK

SEQ ID NO: 23 - protein, 107 aa, synthetic construct
DIQMTQSPSSVSASVGDRVTITCRASQNIGNWLAWYQQKPGKAPKLLIYAASNLQRGVPSRFSGSGSGTDFTLTISSLQPEDFANYYCQQYHNIPITFGGGTKVEIK

SEQ ID NO: 24 - protein, 107 aa, synthetic construct
DIQMTQSPSSVSASVGDRVTITCRASQGIGTYLAWYQQKPGKAPKLLIYDASILGSGVPSRFSGSGSGTDFTLTISSLQPEDFANYYCQHYGTSPPTFGGGTKVEIK

SEQ ID NO: 25 - protein, 112 aa, synthetic construct
DIVMTQSPLSLPVTPGEPASISCRSSQSLLHSTGYNFLHWYLQKPGQSPQLLIYDTFHRATGVPDRFSGSGSGTDFTLKISRVEAEDVGVYYCMQSLQPRFTFGGGTKVEIK

SEQ ID NO: 26 - protein, 112 aa, synthetic construct
DIVMTQSPLSLPVTPGEPASISCRSSQSLLHSTGYNYLHWYLQKPGQSPQLLIYDTSTRASGVPDRFSGSGSGTDFTLKISRVEAEDVGVYYCMQTFHLPFTFGGGTKVEIK

SEQ ID NO: 27 - protein, 112 aa, synthetic construct
DIVMTQSPLSLPVTPGEPASISCRSSQSLLHSSGYNYLHWYLQKPGQSPQLLIYDTTNRATGVPDRFSGSGSGTDFTLKISRVEAEDVGVYYCMQTTQLPYTFGGGTKVEIK

SEQ ID NO: 28 - protein, 107 aa, synthetic construct
DIQMTQSPSSVSASVGDRVTITCRASQGIGRSLAWYQQKPGKAPKLLIYKDTERASGVPSRFSGSGSGTDFTLTISSLQPEDFANYYCQQVHTFPPTFGGGTKVEIK

SEQ ID NO: 29 - protein, 112 aa, synthetic construct
DIVMTQSPLSLPVTPGEPASISCRSSQSLLHVTGYNYLHWYLQKPGQSPQLLIYETSKRAPGVPDRFSGSGSGTDFTLKISRVEAEDVGVYYCMQTTHWPSTFGGGTKVEIK

SEQ ID NO: 30 - protein, 107 aa, synthetic construct
DIQMTQSPSSVSASVGDRVTITCRASQSIGTNLAWYQQKPGKAPKLLIYDASKRPTGVPSRFSGSGSGTDFTLTISSLQPEDFANYYCQQGYDIPLTFGGGTKVEIK

SEQ ID NO: 31 - protein, 107 aa, synthetic construct
DIQMTQSPSSVSASVGDRVTITCRASQNIGDRLAWYQQKPGKAPKLLIYQDRNRPSGVPSRFSGSGSGTDFTLTISSLQPEDFANYYCQQYATSPFTFGGGTKVEIK

SEQ ID NO: 32 - protein, 107 aa, synthetic construct
DIQMTQSPSSVSASVGDRVTITCRASQNIDTYLAWYQQKPGKAPKLLIYEGTKRPSGVPSRFSGSGSGTDFTLTISSLQPEDFANYYCQQWDNLPLTFGGGTKVEIK

SEQ ID NO: 33 - protein, 107 aa, synthetic construct
DIQMTQSPSSVSASVGDRVTITCRASESISSHLAWYQQKPGKAPKLLIYAGSSRATGVPSRFSGSGSGTDFTLTISSLQPEDFANYYCHQYDTLPFTFGGGTKVEIK

SEQ ID NO: 34 - protein, 112 aa, synthetic construct
DIVMTQSPLSLPVTPGEPASISCRSSRSLVHGSGDNYLHWYLQKPGQSPQLLIYMTSNRAPGVPDRFSGSGSGTDFTLKISRVEAEDVGVYYCMQSGHWPPTFGGGTKVEIK

SEQ ID NO: 35 - protein, 107 aa, synthetic construct
DIQMTQSPSSVSASVGDRVTITCRASQNIDTWLAWYQQKPGKAPKLLIYKASTLASGVPSRFSGSGSGTDFTLTISSLQPEDFANYYCQQYNEVPLTFGGGTKVEIK

SEQ ID NO: 36 - protein, 112 aa, synthetic construct
DIVMTQSPLSLPVTPGEPASISCRSSQSLLHSTGYNYLHWYLQKPGQSPQLLIYDATNRASGVPDRFSGSGSGTDFTLKISRVEAEDVGVYYCQQYAASPPTFGGGTKVEIK

SEQ ID NO: 37 - protein, 107 aa, synthetic construct
DIQMTQSPSSVSASVGDRVTITCRASQDISTYLAWYQQKPGKAPKLLIYDTSNRATGIPSRFSGSGSGTDFTLTISSLQPEDFANYYCQQSAKIPFTFGGGTKVEIK

SEQ ID NO: 38 - protein, 107 aa, synthetic construct
EIVMTQSPATLSLSPGERATLSCRASHSVTSNLAWYQQKPGQAPRLLIYGASSRVPGIPARFSGSGSGTDFTLTISSLEPEDFAVYYCQQYDNRPITFGGGTKVEIK

SEQ ID NO: 39 - protein, 106 aa, synthetic construct
DIQMTQSPSSVSASVGDRVTITCRASQSIAPLAWYQQKPGKAPKLLIYAASTLQPGVPSRFSGSGSGTDFTLTISSLQPEDFANYYCLQYHVLPITFGGGTKVEIK

SEQ ID NO: 40 - protein, 107 aa, synthetic construct
EIVMTQSPATLSLSPGERATLSCRASQNIGTNLAWYQQKPGQAPRLLIYDASKRPTGIPARFSGSGSGTDFTLTISSLEPEDFAVYYCQQYGTTPFTFGGGTKVEIK

SEQ ID NO: 41 - protein, 107 aa, synthetic construct
EIVMTQSPATLSLSPGERATLSCRASESIGSNLAWYQQKPGQAPRLLIYDASNRATGIPARFSGSGSGTDFTLTISSLEPEDFAVYYCQQYDHWPLTFGGGTKVEIK

SEQ ID NO: 42 - protein, 112 aa, synthetic construct
DIVMTQSPLSLPVTPGEPASISCRSSRSLVHGSGDNYLHWYLQKPGQSPQLLIYMASNRAPGVPDRFSGSGSGTDFTLKISRVEAEDVGVYYCMQALRAPFSFGGGTKVEIK

SEQ ID NO: 43 - protein, 108 aa, synthetic construct
EIVMTQSPATLSLSPGERATLSCRASQSLSSSHLAWYQQKPGQAPRLLIYDASIRVPGIPARFSGSGSGTDFTLTISSLEPEDFAVYYCQQYAVPPITFGGGTKVEIK

SEQ ID NO: 44 - protein, 107 aa, synthetic construct
DIQMTQSPSSLSASVGDRVTITCRASQGIGSYLAWYQQKPGKAPKLLIYEASRLESGVPSRFSGSGSGTDFTLTISSLQPEDVATYYCQQGYNAPITFGGGTKVEIK

SEQ ID NO: 45 - protein, 107 aa, synthetic construct
DIQMTQSPSSVSASVGDRVTITCRASQDIGNWLAWYQQKPGKAPKLLIYDASTLDTGVPSRFSGSGSGTDFTLTISSLQPEDFANYYCLQDYSYPLTFGGGTKVEIK

SEQ ID NO: 46 - protein, 107 aa, synthetic construct
DIQMTQSPSSLSASVGDRVTITCRASQDISNWLAWYQQKPGKAPKLLIYEASRLESGVPSRFSGSGSGTDFTLTISSLQPEDVATYYCQQGYNAPITFGGGTKVEIK

SEQ ID NO: 47 - protein, 107 aa, synthetic construct
DIQMTQSPSSLSASVGDRVTITCRASQDISNWLAWYQQKPGKAPKLLIYDASSLVSGVPSRFSGSGSGTDFTLTISSLQPEDVATYYCHQIYDTPPTFGGGTKVEIK

SEQ ID NO: 48 - protein, 107 aa, synthetic construct
DIQMTQSPSSLSASVGDRVTITCRASQAISGSLAWYQQKPGKAPKLLIYATSRLESGVPSRFSGSGSGTDFTLTISSLQPEDVATYYCQQSGSTPITFGGGTKVEIK

SEQ ID NO: 49 - protein, 107 aa, synthetic construct
DIQMTQSPSSLSASVGDRVTITCRASQDIAGWLAWYQQKPGKAPKLLIYDASTLQGGVPSRFSGSGSGTDFTLTISSLQPEDVATYYCQQSYTAPLNFGGGTKVEIK

SEQ ID NO: 50 - protein, 107 aa, synthetic construct
EIVMTQSPATLSLSPGERATLSCRASQTIGTNLAWYQQKPGQAPRLLIYDASTRANGIPARFSGSGSGTDFTLTISSLEPEDFAVYYCQQYAAPPLTFGGGTKVEIK

SEQ ID NO: 51 - protein, 108 aa, synthetic construct
EIVMTQSPATLSLSPGERATLSCRASQSIRSDLAWYQQKPGQAPRLLIYDASHRPAGIPARFSGSGSGTDFTLTISSLEPEDFAVYYCQQYGSVPRPTFGGGTKVEIK

SEQ ID NO: 52 - protein, 107 aa, synthetic construct
EIVMTQSPATLSLSPGERATLSCRASQSIRSDLAWYQQKPGQAPRLLIYDATSRAAGIPARFSGSGSGTDFTLTISSLEPEDFAVYYCQEYGSAPLTFGGGTKVEIK

SEQ ID NO: 53 - protein, 107 aa, synthetic construct
EIVMTQSPATLSLSPGERATLSCRASMGVSSNLAWYQQKPGQAPRLLIYDASNRAAGIPARFSGSGSGTDFTLTISSLEPEDFAVYYCQQYAEGPLTFGGGTKVEIK

SEQ ID NO: 54 - protein, 120 aa, synthetic construct
EVQLVESGGGLVQPGGSLRLSCAASAFTFSAHSMSWVRQAPGKGLELVASISPSGSVTYYPDSVKGRFTISRDNAKNSLYLQMNSLRAEDTAVYYCARDSGSYRDAFDIWGQGTMVTVSS

SEQ ID NO: 55 - protein, 119 aa, synthetic construct
EVQLVESGGGLVQPGGSLRLSCAASGFTFAGHAMSWVRQAPGKGLELVASISPSGSTTYYPDSVKGRFTISRDNAKNSLYLQMNSLRAEDTAVYYCARDGDSSDAFDIWGQGTMVTVSS

SEQ ID NO: 56 - protein, 119 aa, synthetic construct
QVQLQESGPGLVKPSQTLSLTCTVSGASISTIDYSWSWIRQPPGKGLEWIGYIDESGRIDYNPSLKSRVTMSVDTSKNQFSLKVNSVTAADTAVYYCAREGQWGQFDYWGQGTLVTVSS

SEQ ID NO: 57 - protein, 119 aa, synthetic construct
EVQLVESGGGLVQPGGSLRLSCAASGFTFSNHAMSWVRQAPGKGLELVASINPSGSVTYYPDSVKGRFTISRDNAKNSLYLQMNSLRAEDTAVYYCARDGDYGDYLDYWGQGTLVTVSS

SEQ ID NO: 58 - protein, 119 aa, synthetic construct
QVQLVQSGAEVKKPGASVKVSCKVSGDTFTSNAIHWVRQAPGKGLEWMGGIVAGSGHTIYAQKFQGRVTMTEDTSTDTAYMELSSLKSEDTAVYYCAREGAEYNGFDPWGQGTLVTVSS

SEQ ID NO: 59 - protein, 119 aa, synthetic construct
QVQLVQSGAEVKKPGASVKVSCKVSGFTFTSSVIHWVRQAPGKGLEWMGGISPRGESTIYAQKFQGRVTMTEDTSTDTAYMELSSLKSEDTAVYYCAREGASTGAFDIWGQGTMVTVSS

SEQ ID NO: 60 - protein, 120 aa, synthetic construct
QVQLVQSGAEVKKPGASVKVSCKVSGYTLTGNAIHWVRQAPGKGLEWMGGIIPSIGTAIYAQKFQGRVTMTEDTSTDTAYMELSSLKSEDTAVYYCAKDRSDIGDIFDYWGQGTLVTVSS

SEQ ID NO: 61 - protein, 121 aa, synthetic construct
EVQLVESGGGLVQPGGSLRLSCAASGFTFTTHSMSWVRQAPGKGLELVASINPAGSITYYPDSVKGRFTISRDNAKNSLYLQMNSLRAEDTAVYYCARDNNYGYGDHFDYWGQGTLVTVSS

SEQ ID NO: 62 - protein, 118 aa, synthetic construct
EVQLVESGGGLVQPGGSLRLSCAASGFTFGDHVMSWVRQAPGKGLELVASISPSGDVTYYPDSVKGRFTISRDNAKNSLYLQMNSLRAEDTAVYYCTTDGDSDAFDIWGQGTMVTVSS

SEQ ID NO: 63 - protein, 118 aa, synthetic construct
EVQLVESGGGLVQPGGSLRLSCAASGLTFSNHAMSWVRQAPGKGLELVASINPSGSVTYYPDSVKGRFTISRDNAKNSLYLQMNSLRAEDTAVYYCTADDYGDYLDYWGQGTLVTVSS

SEQ ID NO: 64 - protein, 119 aa, synthetic construct
QVQLVQSGAEVKKPGASVKVSCKVSGYTFSNYVIHWVRQAPGKGLEWMGGIVAGSGHTIYAQKFQGRVTMTEDTSTDTAYMELSSLKSEDTAVYYCATESAAGNWFDPWGQGTLVTVSS

SEQ ID NO: 65 - protein, 119 aa, synthetic construct
EVQLVESGGGLVQPGGSLRLSCAASGFTFSDHTMSWVRQAPGKGLELVASINPSGSVTYYPDSVKGRFTISRDNAKNSLYLQMNSLRAEDTAVYYCARDGGYGDYFDYWGQGTLVTVSS

SEQ ID NO: 66 - protein, 120 aa, synthetic construct
QVQLVQSGAEVKKPGASVKVSCKVSGTPLPATIHWVRQAPGKGLEWMGGINPSGATIYAQKFQGRVTMTEDTSTDTAYMELSSLKSEDTAVYYCAKDSDVAAAGSFFDYWGQGTLVTVSS

SEQ ID NO: 67 - protein, 121 aa, synthetic construct
QVQLVQSGAEVKKPGASVKVSCKVSGYTFRNYAIHWVRQAPGKGLEWMGGISPSGSTTIYAQKFQGRVTMTEDTSTDTAYMELSSLKSEDTAVYYCARESAENYGDYLDYWGQGTLVTVSS

SEQ ID NO: 68 - protein, 119 aa, synthetic construct
QVQLVQSGAEVKKPGASVKVSCKVSGIDLTTSAIHWVRQAPGKGLEWMGGIAIGSGHTIYAQKFQGRVTMTEDTSTDTAYMELSSLKSEDTAVYYCARDGNFGDYIEYWGQGTLVTVSS

SEQ ID NO: 69 - protein, 119 aa, synthetic construct
QVQLVQSGAEVKKPGASVKVSCKVSGHTFTGYFIHWVRQAPGKGLEWMGGMDPISGATIYAQKFQGRVTMTEDTSTDTAYMELSSLKSEDTAVYYCAREGTTIDAFDIWGQGTMVTVSS

SEQ ID NO: 70 - protein, 121 aa, synthetic construct
EVQLVESGGGLVQPGGSLRLSCAASGLMFSTSTMSWVRQAPGKGLELVASINPSGSVTYYPDSVKGRFTISRDNAKNSLYLQMNSLRAEDTAVYYCAREDGDSRGEAFDIWGQGTMVTVSS

SEQ ID NO: 71 - protein, 119 aa, synthetic construct
QVQLVQSGAEVKKPGASVKVSCKVSGIDLTTSAIHWVRQAPGKGLEWMGGINPGTGSTIYAQKFQGRVTMTEDTSTDTAYMELSSLKSEDTAVYYCAKEVAATGAYYYWGQGTLVTVSS

SEQ ID NO: 72 - protein, 119 aa, synthetic construct
EVQLVESGGGLVQPGGSLRLSCAASGFPFSDYVMSWVRQAPGKGLELVASISPSGSTTYYPDSVKGRFTISRDNAKNSLYLQMNSLRAEDTAVYYCVRSGEWTDAFDIWGQGTLVTVSS

SEQ ID NO: 73 - protein, 120 aa, synthetic construct
QVQLVQSGAEVKKPGASVKVSCKVSGYTFSDYVIHWVRQAPGKGLEWMGGINPYSGHTIYAQKFQGRVTMTEDTSTDTAYMELSSLKSEDTAVYYCAKELDTAGNAFDIWGQGTMVTVSS

SEQ ID NO: 74 - protein, 117 aa, synthetic construct
QVQLQESGPGLVKPSQTLSLTCTVSGASVRDHYWSWIRQPPGKGLEWIGYAHYSGITDYNPSLKSRVTMSVDTSKNQFSLKVNSVTAADTAVYYCAIYSSGWWEDYWGQGTLVTVSS

SEQ ID NO: 75 - protein, 120 aa, synthetic construct
QVQLVQSGAEVKKPGASVKVSCKVSGYPFPSYDIHWVRQAPGKGLEWMGGMNPTTGDTIYAQKFQGRVTMTEDTSTDTAYMELSSLKSEDTAVYYCARETGGGEMAFDIWGQGTMVTVSS

SEQ ID NO: 76 - protein, 122 aa, synthetic construct
QVQLQESGPGLVKPSQTLSLTCTVSEYAIRSDYTWSWIRQPPGKGLEWIGYIHHSGLTDYNPSLKSRVTMSVDTSKNQFSLKVNSVTAADTAVYYCARVRYSSSSEGWFDPWGQGTLVTVSS

SEQ ID NO: 77 - protein, 121 aa, synthetic construct
QVQLQESGPGLVKPSQTLSLTCTVSGASISTSDTWSWIRQPPGKGLEWIGYIHHSGITDYNPSLKSRVTMSVDTSKNQFSLKVNSVTAADTAVYYCARGGSSGNWYLLDYWGQGTLVTVSS

SEQ ID NO: 78 - protein, 117 aa, synthetic construct
EVQLVESGGGLVQPGGSLRLSCAASRFTFSDYHMSWVRQAPGKGLELVASIDTEGKTYYPDSVKGRFTISRDNAKNSLYLQMNSLRAEDTAVYYCARDVGDWYFDLWGRGTLVTVSS

SEQ ID NO: 79 - protein, 118 aa, synthetic construct
QVQLQESGPGLVKPSQTLSLTCTVSGVSLSNARMGWSWIRQPPGKGLEWIGYVHTSGSTDYNPSLKSRVTMSVDTSKNQFSLKVNSVTAADTAVYYCARDAGNWFDPWGQGTLVTVSS

SEQ ID NO: 80 - protein, 118 aa, synthetic construct
EVQLVESGGGLVQPGRSLRLSCAASGSTLSSYGMHWVRQAPGKGLEWVSAISYDASTIDYADSVEGRFTISRDNAKNSLYLQMNSLRAEDTAVYYCARDLLGYGMDVWGQGTMVTVSS

SEQ ID NO: 81 - protein, 123 aa, synthetic construct
QVQLVQSGAEVKKPGASVKVSCKVSGSAFTSNDIHWVRQAPGKGLEWMGGINPRSGATIYAQKFQGRVTMTEDTSTDTAYMELSSLKSEDTAVYYCARALSDDSSGYDAFDIWGQGTMVTVSS

SEQ ID NO: 82 - protein, 120 aa, synthetic construct
EVQLVESGGGLVQPGRSLRLSCAASGFNFGAFAMHWVRQAPGKGLEWVSAINQGGSEVDYADSVEGRFTISRDNAKNSLYLQMNSLRAEDTAVYYCARDPGLPVSAFDIWGLGTMVTVSS

SEQ ID NO: 83 - protein, 117 aa, synthetic construct
EVQLVESGGGLVQPGRSLRLSCAASGFTFENYGMHWVRQAPGKGLEWVSAISYDASTIDYADSVEGRFTISRDNAKNSLYLQMNSLRAEDTAVYYCASEYGLAFDQWGQGTLVTVSS

SEQ ID NO: 84 - protein, 117 aa, synthetic construct
EVQLVESGGGLVQPGRSLRLSCAASGSTLSSYGMHWVRQAPGKGLEWVSAISYDASTIDYADSVEGRFTISRDNAKNSLYLQMNSLRAEDTAVYYCASEYGLAFDQWGQGTLVTVSS

SEQ ID NO: 85 - protein, 118 aa, synthetic construct
EVQLVESGGGLVQPGRSLRLSCAASGLTFSNHGMHWVRQAPGKGLEWVSAISSSADIVDYADSVEGRFTISRDNAKNSLYLQMNSLRAEDTAVYYCASEGVPYGMDVWGQGTMVTVSS

SEQ ID NO: 86 - protein, 118 aa, synthetic construct
EVQLVESGGGLVQPGRSLRLSCAASGSTLSSYGMHWVRQAPGKGLEWVSAISYDASTIDYADSVEGRFTISRDNAKNSLYLQMNSLRAEDTAVYYCASEGVPYGMDVWGQGTMVTVSS

SEQ ID NO: 87 - protein, 117 aa, synthetic construct
QVQLQESGPGLVKPSQTLSLTCTVSGESFSDFYWSWIRQPPGKGLEWIGYIDHTGSTDYNPSLKSRVTMSVDTSKNQFSLKVNSVTAADTAVYYCAGDKYADGFDVWGQGTMVTVSS

SEQ ID NO: 88 - protein, 127 aa, synthetic construct
QVQLQESGPGLVKPSQTLSLTCTVSGFSLSTSGVRWSWIRQPPGKGLEWIGYINPSGTTDYNPSLKSRVTMSVDTSKNQFSLKVNSVTAADTAVYYCARGGGYCSGGSCYDWYFDLWGRGTLVTVSS

SEQ ID NO: 89 - protein, 121 aa, synthetic construct
QVQLQESGPGLVKPSQTLSLTCTVSGESFSGYSWSWIRQPPGKGLEWIGYITHSGTIDYNPSLKSRVTMSVDTSKNQFSLKVNSVTAADTAVYYCAKPLDFGDYKDAFDIWGQGTMVTVSS

SEQ ID NO: 90 - protein, 117 aa, synthetic construct
EVQLVESGGGLVQPGRSLRLSCAASGSTLSSYGMHWVRQAPGKGLEWVSAISYDASTIDYADSVEGRFTISRDNAKNSLYLQMNSLRAEDTAVYYCARDGISGIDYWGQGTLVTVSS

SEQ ID NO: 91 - protein, 252 aa, synthetic construct
DIVMTQSPLSLPVTPGEPASISCRSSQSLVHGSGYTFLHWYLQKPGQSPQLLIYDAVTRATGVPDRFSGSGSGTDFTLKISRVEAEDVGVYYCMQTTEFPYTFGGGTKVEIKGGGGSGGGGSGGGGSGGGGSEVQLVESGGGLVQPGGSLRLSCAASAFTFSAHSMSWVRQAPGKGLELVASISPSGSVTYYPDSVKGRFTISRDNAKNSLYLQMNSLRAEDTAVYYCARDSGSYRDAFDIWGQGTMVTVSS

SEQ ID NO: 92 - protein, 251 aa, synthetic construct
DIVMTQSPLSLPVTPGEPASISCRSSQSLLHSTGYNFLHWYLQKPGQSPQLLIYDASKRAPGVPDRFSGSGSGTDFTLKISRVEAEDVGVYYCMQTVELPFTFGGGTKVEIKGGGGSGGGGSGGGGSGGGGSEVQLVESGGGLVQPGGSLRLSCAASGFTFAGHAMSWVRQAPGKGLELVASISPSGSTTYYPDSVKGRFTISRDNAKNSLYLQMNSLRAEDTAVYYCARDGDSSDAFDIWGQGTMVTVSS

SEQ ID NO: 93 - protein, 246 aa, synthetic construct
EIVMTQSPATLSLSPGERATLSCRASQSVDRHLAWYQQKPGQAPRLLIYDVSNRAPGIPARFSGSGSGTDFTLTISSLEPEDFAVYYCQQYGWPSITFGGGTKVEIKGGGGSGGGGSGGGGSGGGGSQVQLQESGPGLVKPSQTLSLTCTVSGASISTIDYSWSWIRQPPGKGLEWIGYIDESGRIDYNPSLKSRVTMSVDTSKNQFSLKVNSVTAADTAVYYCAREGQWGQFDYWGQGTLVTVSS

SEQ ID NO: 94 - protein, 251 aa, synthetic construct
DIVMTQSPLSLPVTPGEPASISCRSSQSLLSSYGYHNLHWYLQKPGQSPQLLIYDAYVRATGVPDRFSGSGSGTDFTLKISRVEAEDVGVYYCMQSKELPYTFGGGTKVEIKGGGGSGGGGSGGGGSGGGGSEVQLVESGGGLVQPGGSLRLSCAASGFTFSNHAMSWVRQAPGKGLELVASINPSGSVTYYPDSVKGRFTISRDNAKNSLYLQMNSLRAEDTAVYYCARDGDYGDYLDYWGQGTLVTVSS

SEQ ID NO: 95 - protein, 247 aa, synthetic construct
DIQMTQSPSSVSASVGDRVTITCRASQDIGSNLAWYQQKPGKAPKLLIYSGSTLQSGVPSRFSGSGSGTDFTLTISSLQPEDFANYYCQQTSSSPPYTFGGGTKVEIKGGGGSGGGGSGGGGSGGGGSQVQLVQSGAEVKKPGASVKVSCKVSGDTFTSNAIHWVRQAPGKGLEWMGGIVAGSGHTIYAQKFQGRVTMTEDTSTDTAYMELSSLKSEDTAVYYCAREGAEYNGFDPWGQGTLVTVSS

SEQ ID NO: 96 - protein, 246 aa, synthetic construct
DIQMTQSPSSVSASVGDRVTITCRASQNIGNWLAWYQQKPGKAPKLLIYAASNLQRGVPSRFSGSGSGTDFTLTISSLQPEDFANYYCQQYHNIPITFGGGTKVEIKGGGGSGGGGSGGGGSGGGGSQVQLVQSGAEVKKPGASVKVSCKVSGFTFTSSVIHWVRQAPGKGLEWMGGISPRGESTIYAQKFQGRVTMTEDTSTDTAYMELSSLKSEDTAVYYCAREGASTGAFDIWGQGTMVTVSS

SEQ ID NO: 97 - protein, 247 aa, synthetic construct
DIQMTQSPSSVSASVGDRVTITCRASQGIGTYLAWYQQKPGKAPKLLIYDASILGSGVPSRFSGSGSGTDFTLTISSLQPEDFANYYCQHYGTSPPTFGGGTKVEIKGGGGSGGGGSGGGGSGGGGSQVQLVQSGAEVKKPGASVKVSCKVSGYTLTGNAIHWVRQAPGKGLEWMGGIIPSIGTAIYAQKFQGRVTMTEDTSTDTAYMELSSLKSEDTAVYYCAKDRSDIGDIFDYWGQGTLVTVSS

SEQ ID NO: 98 - protein, 253 aa, synthetic construct
DIVMTQSPLSLPVTPGEPASISCRSSQSLLHSTGYNFLHWYLQKPGQSPQLLIYDTFHRATGVPDRFSGSGSGTDFTLKISRVEAEDVGVYYCMQSLQPRFTFGGGTKVEIKGGGGSGGGGSGGGGSGGGGSEVQLVESGGGLVQPGGSLRLSCAASGFTFTTHSMSWVRQAPGKGLELVASINPAGSITYYPDSVKGRFTISRDNAKNSLYLQMNSLRAEDTAVYYCARDNNYGYGDHFDYWGQGTLVTVSS

SEQ ID NO: 99 - protein, 250 aa, synthetic construct
DIVMTQSPLSLPVTPGEPASISCRSSQSLLHSTGYNYLHWYLQKPGQSPQLLIYDTSTRASGVPDRFSGSGSGTDFTLKISRVEAEDVGVYYCMQTFHLPFTFGGGTKVEIKGGGGSGGGGSGGGGSGGGGSEVQLVESGGGLVQPGGSLRLSCAASGFTFGDHVMSWVRQAPGKGLELVASISPSGDVTYYPDSVKGRFTISRDNAKNSLYLQMNSLRAEDTAVYYCTTDGDSDAFDIWGQGTMVTVSS

SEQ ID NO: 100 - protein, 250 aa, synthetic construct
DIVMTQSPLSLPVTPGEPASISCRSSQSLLHSSGYNYLHWYLQKPGQSPQLLIYDTTNRATGVPDRFSGSGSGTDFTLKISRVEAEDVGVYYCMQTTQLPYTFGGGTKVEIKGGGGSGGGGSGGGGSGGGGSEVQLVESGGGLVQPGGSLRLSCAASGLTFSNHAMSWVRQAPGKGLELVASINPSGSVTYYPDSVKGRFTISRDNAKNSLYLQMNSLRAEDTAVYYCTADDYGDYLDYWGQGTLVTVSS

SEQ ID NO: 101 - protein, 246 aa, synthetic construct
DIQMTQSPSSVSASVGDRVTITCRASQGIGRSLAWYQQKPGKAPKLLIYKDTERASGVPSRFSGSGSGTDFTLTISSLQPEDFANYYCQQVHTFPPTFGGGTKVEIKGGGGSGGGGSGGGGSGGGGSQVQLVQSGAEVKKPGASVKVSCKVSGYTFSNYVIHWVRQAPGKGLEWMGGIVAGSGHTIYAQKFQGRVTMTEDTSTDTAYMELSSLKSEDTAVYYCATESAAGNWFDPWGQGTLVTVSS

SEQ ID NO: 102 - protein, 251 aa, synthetic construct
DIVMTQSPLSLPVTPGEPASISCRSSQSLLHVTGYNYLHWYLQKPGQSPQLLIYETSKRAPGVPDRFSGSGSGTDFTLKISRVEAEDVGVYYCMQTTHWPSTFGGGTKVEIKGGGGSGGGGSGGGGSGGGGSEVQLVESGGGLVQPGGSLRLSCAASGFTFSDHTMSWVRQAPGKGLELVASINPSGSVTYYPDSVKGRFTISRDNAKNSLYLQMNSLRAEDTAVYYCARDGGYGDYFDYWGQGTLVTVSS

SEQ ID NO: 103 - protein, 247 aa, synthetic construct
DIQMTQSPSSVSASVGDRVTITCRASQSIGTNLAWYQQKPGKAPKLLIYDASKRPTGVPSRFSGSGSGTDFTLTISSLQPEDFANYYCQQGYDIPLTFGGGTKVEIKGGGGSGGGGSGGGGSGGGGSQVQLVQSGAEVKKPGASVKVSCKVSGTPLPATIHWVRQAPGKGLEWMGGINPSGATIYAQKFQGRVTMTEDTSTDTAYMELSSLKSEDTAVYYCAKDSDVAAAGSFFDYWGQGTLVTVSS

SEQ ID NO: 104 - protein, 248 aa, synthetic construct
DIQMTQSPSSVSASVGDRVTITCRASQNIGDRLAWYQQKPGKAPKLLIYQDRNRPSGVPSRFSGSGSGTDFTLTISSLQPEDFANYYCQQYATSPFTFGGGTKVEIKGGGGSGGGGSGGGGSGGGGSQVQLVQSGAEVKKPGASVKVSCKVSGYTFRNYAIHWVRQAPGKGLEWMGGISPSGSTTIYAQKFQGRVTMTEDTSTDTAYMELSSLKSEDTAVYYCARESAENYGDYLDYWGQGTLVTVSS

SEQ ID NO: 105 - protein, 246 aa, synthetic construct
DIQMTQSPSSVSASVGDRVTITCRASQNIDTYLAWYQQKPGKAPKLLIYEGTKRPSGVPSRFSGSGSGTDFTLTISSLQPEDFANYYCQQWDNLPLTFGGGTKVEIKGGGGSGGGGSGGGGSGGGGSQVQLVQSGAEVKKPGASVKVSCKVSGIDLTTSAIHWVRQAPGKGLEWMGGIAIGSGHTIYAQKFQGRVTMTEDTSTDTAYMELSSLKSEDTAVYYCARDGNFGDYIEYWGQGTLVTVSS

SEQ ID NO: 106 - protein, 246 aa, synthetic construct
DIQMTQSPSSVSASVGDRVTITCRASESISSHLAWYQQKPGKAPKLLIYAGSSRATGVPSRFSGSGSGTDFTLTISSLQPEDFANYYCHQYDTLPFTFGGGTKVEIKGGGGSGGGGSGGGGSGGGGSQVQLVQSGAEVKKPGASVKVSCKVSGHTFTGYFIHWVRQAPGKGLEWMGGMDPISGATIYAQKFQGRVTMTEDTSTDTAYMELSSLKSEDTAVYYCAREGTTIDAFDIWGQGTMVTVSS

SEQ ID NO: 107 - protein, 253 aa, synthetic construct
DIVMTQSPLSLPVTPGEPASISCRSSRSLVHGSGDNYLHWYLQKPGQSPQLLIYMTSNRAPGVPDRFSGSGSGTDFTLKISRVEAEDVGVYYCMQSGHWPPTFGGGTKVEIKGGGGSGGGGSGGGGSGGGGSEVQLVESGGGLVQPGGSLRLSCAASGLMFSTSTMSWVRQAPGKGLELVASINPSGSVTYYPDSVKGRFTISRDNAKNSLYLQMNSLRAEDTAVYYCAREDGDSRGEAFDIWGQGTMVTVSS

SEQ ID NO: 108 - protein, 246 aa, synthetic construct
DIQMTQSPSSVSASVGDRVTITCRASQNIDTWLAWYQQKPGKAPKLLIYKASTLASGVPSRFSGSGSGTDFTLTISSLQPEDFANYYCQQYNEVPLTFGGGTKVEIKGGGGSGGGGSGGGGSGGGGSQVQLVQSGAEVKKPGASVKVSCKVSGIDLTTSAIHWVRQAPGKGLEWMGGINPGTGSTIYAQKFQGRVTMTEDTSTDTAYMELSSLKSEDTAVYYCAKEVAATGAYYYWGQGTLVTVSS

SEQ ID NO: 109 - protein, 251 aa, synthetic construct
DIVMTQSPLSLPVTPGEPASISCRSSQSLLHSTGYNYLHWYLQKPGQSPQLLIYDATNRASGVPDRFSGSGSGTDFTLKISRVEAEDVGVYYCQQYAASPPTFGGGTKVEIKGGGGSGGGGSGGGGSGGGGSEVQLVESGGGLVQPGGSLRLSCAASGFPFSDYVMSWVRQAPGKGLELVASISPSGSTTYYPDSVKGRFTISRDNAKNSLYLQMNSLRAEDTAVYYCVRSGEWTDAFDIWGQGTLVTVSS

SEQ ID NO: 110 - protein, 247 aa, synthetic construct
DIQMTQSPSSVSASVGDRVTITCRASQDISTYLAWYQQKPGKAPKLLIYDTSNRATGIPSRFSGSGSGTDFTLTISSLQPEDFANYYCQQSAKIPFTFGGGTKVEIKGGGGSGGGGSGGGGSGGGGSQVQLVQSGAEVKKPGASVKVSCKVSGYTFSDYVIHWVRQAPGKGLEWMGGINPYSGHTIYAQKFQGRVTMTEDTSTDTAYMELSSLKSEDTAVYYCAKELDTAGNAFDIWGQGTMVTVSS

SEQ ID NO: 111 - protein, 244 aa, synthetic construct
EIVMTQSPATLSLSPGERATLSCRASHSVTSNLAWYQQKPGQAPRLLIYGASSRVPGIPARFSGSGSGTDFTLTISSLEPEDFAVYYCQQYDNRPITFGGGTKVEIKGGGGSGGGGSGGGGSGGGGSQVQLQESGPGLVKPSQTLSLTCTVSGASVRDHYWSWIRQPPGKGLEWIGYAHYSGITDYNPSLKSRVTMSVDTSKNQFSLKVNSVTAADTAVYYCAIYSSGWWEDYWGQGTLVTVSS

SEQ ID NO: 112 - protein, 246 aa, synthetic construct
DIQMTQSPSSVSASVGDRVTITCRASQSIAPLAWYQQKPGKAPKLLIYAASTLQPGVPSRFSGSGSGTDFTLTISSLQPEDFANYYCLQYHVLPITFGGGTKVEIKGGGGSGGGGSGGGGSGGGGSQVQLVQSGAEVKKPGASVKVSCKVSGYPFPSYDIHWVRQAPGKGLEWMGGMNPTTGDTIYAQKFQGRVTMTEDTSTDTAYMELSSLKSEDTAVYYCARETGGGEMAFDIWGQGTMVTVSS

SEQ ID NO: 113 - protein, 249 aa, synthetic construct
EIVMTQSPATLSLSPGERATLSCRASQNIGTNLAWYQQKPGQAPRLLIYDASKRPTGIPARFSGSGSGTDFTLTISSLEPEDFAVYYCQQYGTTPFTFGGGTKVEIKGGGGSGGGGSGGGGSGGGGSQVQLQESGPGLVKPSQTLSLTCTVSEYAIRSDYTWSWIRQPPGKGLEWIGYIHHSGLTDYNPSLKSRVTMSVDTSKNQFSLKVNSVTAADTAVYYCARVRYSSSSEGWFDPWGQGTLVTVSS

SEQ ID NO: 114 - protein, 248 aa, synthetic construct
EIVMTQSPATLSLSPGERATLSCRASESIGSNLAWYQQKPGQAPRLLIYDASNRATGIPARFSGSGSGTDFTLTISSLEPEDFAVYYCQQYDHWPLTFGGGTKVEIKGGGGSGGGGSGGGGSGGGGSQVQLQESGPGLVKPSQTLSLTCTVSGASISTSDTWSWIRQPPGKGLEWIGYIHHSGITDYNPSLKSRVTMSVDTSKNQFSLKVNSVTAADTAVYYCARGGSSGNWYLLDYWGQGTLVTVSS

SEQ ID NO: 115 - protein, 249 aa, synthetic construct
DIVMTQSPLSLPVTPGEPASISCRSSRSLVHGSGDNYLHWYLQKPGQSPQLLIYMASNRAPGVPDRFSGSGSGTDFTLKISRVEAEDVGVYYCMQALRAPFSFGGGTKVEIKGGGGSGGGGSGGGGSGGGGSEVQLVESGGGLVQPGGSLRLSCAASRFTFSDYHMSWVRQAPGKGLELVASIDTEGKTYYPDSVKGRFTISRDNAKNSLYLQMNSLRAEDTAVYYCARDVGDWYFDLWGRGTLVTVSS

SEQ ID NO: 116 - protein, 246 aa, synthetic construct
EIVMTQSPATLSLSPGERATLSCRASQSLSSSHLAWYQQKPGQAPRLLIYDASIRVPGIPARFSGSGSGTDFTLTISSLEPEDFAVYYCQQYAVPPITFGGGTKVEIKGGGGSGGGGSGGGGSGGGGSQVQLQESGPGLVKPSQTLSLTCTVSGVSLSNARMGWSWIRQPPGKGLEWIGYVHTSGSTDYNPSLKSRVTMSVDTSKNQFSLKVNSVTAADTAVYYCARDAGNWFDPWGQGTLVTVSS

SEQ ID NO: 117 - protein, 245 aa, synthetic construct
DIQMTQSPSSLSASVGDRVTITCRASQGIGSYLAWYQQKPGKAPKLLIYEASRLESGVPSRFSGSGSGTDFTLTISSLQPEDVATYYCQQGYNAPITFGGGTKVEIKGGGGSGGGGSGGGGSGGGGSEVQLVESGGGLVQPGRSLRLSCAASGSTLSSYGMHWVRQAPGKGLEWVSAISYDASTIDYADSVEGRFTISRDNAKNSLYLQMNSLRAEDTAVYYCARDLLGYGMDVWGQGTMVTVSS

SEQ ID NO: 118 - protein, 250 aa, synthetic construct
DIQMTQSPSSVSASVGDRVTITCRASQDIGNWLAWYQQKPGKAPKLLIYDASTLDTGVPSRFSGSGSGTDFTLTISSLQPEDFANYYCLQDYSYPLTFGGGTKVEIKGGGGSGGGGSGGGGSGGGGSQVQLVQSGAEVKKPGASVKVSCKVSGSAFTSNDIHWVRQAPGKGLEWMGGINPRSGATIYAQKFQGRVTMTEDTSTDTAYMELSSLKSEDTAVYYCARALSDDSSGYDAFDIWGQGTMVTVSS

SEQ ID NO: 119 - protein, 245 aa, synthetic construct
DIQMTQSPSSLSASVGDRVTITCRASQDISNWLAWYQQKPGKAPKLLIYEASRLESGVPSRFSGSGSGTDFTLTISSLQPEDVATYYCQQGYNAPITFGGGTKVEIKGGGGSGGGGSGGGGSGGGGSEVQLVESGGGLVQPGRSLRLSCAASGSTLSSYGMHWVRQAPGKGLEWVSAISYDASTIDYADSVEGRFTISRDNAKNSLYLQMNSLRAEDTAVYYCARDLLGYGMDVWGQGTMVTVSS

SEQ ID NO: 120 - protein, 247 aa, synthetic construct
DIQMTQSPSSLSASVGDRVTITCRASQDISNWLAWYQQKPGKAPKLLIYDASSLVSGVPSRFSGSGSGTDFTLTISSLQPEDVATYYCHQIYDTPPTFGGGTKVEIKGGGGSGGGGSGGGGSGGGGSEVQLVESGGGLVQPGRSLRLSCAASGFNFGAFAMHWVRQAPGKGLEWVSAINQGGSEVDYADSVEGRFTISRDNAKNSLYLQMNSLRAEDTAVYYCARDPGLPVSAFDIWGLGTMVTVSS

SEQ ID NO: 121 - protein, 244 aa, synthetic construct
DIQMTQSPSSLSASVGDRVTITCRASQAISGSLAWYQQKPGKAPKLLIYATSRLESGVPSRFSGSGSGTDFTLTISSLQPEDVATYYCQQSGSTPITFGGGTKVEIKGGGGSGGGGSGGGGSGGGGSEVQLVESGGGLVQPGRSLRLSCAASGFTFENYGMHWVRQAPGKGLEWVSAISYDASTIDYADSVEGRFTISRDNAKNSLYLQMNSLRAEDTAVYYCASEYGLAFDQWGQGTLVTVSS

SEQ ID NO: 122 - protein, 244 aa, synthetic construct
DIQMTQSPSSLSASVGDRVTITCRASQGIGSYLAWYQQKPGKAPKLLIYEASRLESGVPSRFSGSGSGTDFTLTISSLQPEDVATYYCQQGYNAPITFGGGTKVEIKGGGGSGGGGSGGGGSGGGGSEVQLVESGGGLVQPGRSLRLSCAASGSTLSSYGMHWVRQAPGKGLEWVSAISYDASTIDYADSVEGRFTISRDNAKNSLYLQMNSLRAEDTAVYYCASEYGLAFDQWGQGTLVTVSS

SEQ ID NO: 123 - protein, 245 aa, synthetic construct
DIQMTQSPSSLSASVGDRVTITCRASQDIAGWLAWYQQKPGKAPKLLIYDASTLQGGVPSRFSGSGSGTDFTLTISSLQPEDVATYYCQQSYTAPLNFGGGTKVEIKGGGGSGGGGSGGGGSGGGGSEVQLVESGGGLVQPGRSLRLSCAASGLTFSNHGMHWVRQAPGKGLEWVSAISSSADIVDYADSVEGRFTISRDNAKNSLYLQMNSLRAEDTAVYYCASEGVPYGMDVWGQGTMVTVSS

SEQ ID NO: 124 - protein, 245 aa, synthetic construct
DIQMTQSPSSLSASVGDRVTITCRASQGIGSYLAWYQQKPGKAPKLLIYEASRLESGVPSRFSGSGSGTDFTLTISSLQPEDVATYYCQQGYNAPITFGGGTKVEIKGGGGSGGGGSGGGGSGGGGSEVQLVESGGGLVQPGRSLRLSCAASGSTLSSYGMHWVRQAPGKGLEWVSAISYDASTIDYADSVEGRFTISRDNAKNSLYLQMNSLRAEDTAVYYCASEGVPYGMDVWGQGTMVTVSS

SEQ ID NO: 125 - protein, 244 aa, synthetic construct
EIVMTQSPATLSLSPGERATLSCRASQTIGTNLAWYQQKPGQAPRLLIYDASTRANGIPARFSGSGSGTDFTLTISSLEPEDFAVYYCQQYAAPPLTFGGGTKVEIKGGGGSGGGGSGGGGSGGGGSQVQLQESGPGLVKPSQTLSLTCTVSGESFSDFYWSWIRQPPGKGLEWIGYIDHTGSTDYNPSLKSRVTMSVDTSKNQFSLKVNSVTAADTAVYYCAGDKYADGFDVWGQGTMVTVSS

SEQ ID NO: 126 - protein, 255 aa, synthetic construct
EIVMTQSPATLSLSPGERATLSCRASQSIRSDLAWYQQKPGQAPRLLIYDASHRPAGIPARFSGSGSGTDFTLTISSLEPEDFAVYYCQQYGSVPRPTFGGGTKVEIKGGGGSGGGGSGGGGSGGGGSQVQLQESGPGLVKPSQTLSLTCTVSGFSLSTSGVRWSWIRQPPGKGLEWIGYINPSGTTDYNPSLKSRVTMSVDTSKNQFSLKVNSVTAADTAVYYCARGGGYCSGGSCYDWYFDLWGRGTLVTVSS

SEQ ID NO: 127 - protein, 254 aa, synthetic construct
EIVMTQSPATLSLSPGERATLSCRASQSIRSDLAWYQQKPGQAPRLLIYDATSRAAGIPARFSGSGSGTDFTLTISSLEPEDFAVYYCQEYGSAPLTFGGGTKVEIKGGGGSGGGGSGGGGSGGGGSQVQLQESGPGLVKPSQTLSLTCTVSGFSLSTSGVRWSWIRQPPGKGLEWIGYINPSGTTDYNPSLKSRVTMSVDTSKNQFSLKVNSVTAADTAVYYCARGGGYCSGGSCYDWYFDLWGRGTLVTVSS

SEQ ID NO: 128 - protein, 248 aa, synthetic construct
EIVMTQSPATLSLSPGERATLSCRASMGVSSNLAWYQQKPGQAPRLLIYDASNRAAGIPARFSGSGSGTDFTLTISSLEPEDFAVYYCQQYAEGPLTFGGGTKVEIKGGGGSGGGGSGGGGSGGGGSQVQLQESGPGLVKPSQTLSLTCTVSGESFSGYSWSWIRQPPGKGLEWIGYITHSGTIDYNPSLKSRVTMSVDTSKNQFSLKVNSVTAADTAVYYCAKPLDFGDYKDAFDIWGQGTMVTVSS

SEQ ID NO: 129 - protein, 244 aa, synthetic construct
DIQMTQSPSSLSASVGDRVTITCRASQGIGSYLAWYQQKPGKAPKLLIYEASRLESGVPSRFSGSGSGTDFTLTISSLQPEDVATYYCQQGYNAPITFGGGTKVEIKGGGGSGGGGSGGGGSGGGGSEVQLVESGGGLVQPGRSLRLSCAASGSTLSSYGMHWVRQAPGKGLEWVSAISYDASTIDYADSVEGRFTISRDNAKNSLYLQMNSLRAEDTAVYYCARDGISGIDYWGQGTLVTVSS

SEQ ID NO: 130 - protein, 116 aa, synthetic construct
EVQLLESGGGLVQPGGSLRLSCAASGFTFSDYWMSWVRQAPGKGLEWVSSIYSGTTYIYYADSVKGRFTISRDNSENTLYLQMNSLRAEDTAVYYCTRGWTYFDYWGQGTLVTVSS

SEQ ID NO: 131 - protein, 122 aa, synthetic construct
EVQLLESGGGLVQPGGSLRLSCAASGFTFSDHYMSWVRQAPGRGLEWVSTISSGGSYIYYADSVKGRFTISRDNSKNTLYLQMNSLRAEDTAVYYCAKILKNGNYIYYFDYWGQGTLVTVSS

SEQ ID NO: 132 - protein, 120 aa, synthetic construct
EVQLLESGGGLVQPGGSLRLSCAASGFTFTGYDMTWVRQAPGKGLEWVSAISWNTGSTTYYADSVKGRFTISRDNSKNTLYLQMNSLRAEDTAVYYCTRGYDRKNYFEYWGQGTLVTVSS

SEQ ID NO: 133 - protein, 122 aa, synthetic construct
EVQLLESGGGLVQPGGSLRLSCAASGFTFSDHYMSWVRQAPGKGLEWVSTISSGGSYIYYADSVKGRFTISRDNSKNTLYLQMNSLRAEDTAVYYCAKILKNGNYIYYFDYWGQGTLVTVSS

SEQ ID NO: 134 - protein, 117 aa, synthetic construct
EVQLLESGGGLVQPGGSLRLSCVASGFTFDDYGMTWVRQASGKGLEFVATVNWNGNKTYYADSMKGRFTISRNNSENTVYLEVNSLRDEDTAIYYCVKNHEWKFEYWGQGTLVTVSS

SEQ ID NO: 135 - protein, 116 aa, synthetic construct
EVQLLESGGGLVQPGGSLRLSCAASGFTFSDYWMSWVRQAPGKGLEWVSSIYSGPSYIYYADSVKGRFTISRDNSKNTLYLQMNSLRAEDTAVYYCTRGWTYFDYWGQGTLVTVSS

SEQ ID NO: 136 - protein, 118 aa, synthetic construct
EVQLLESGGGLVQPGGSLRLSCAASGFTFSSDAMNWVRQAPGKGLEWVSTIYSGGSTYYADSVKGRFTISRDNSKNTLYLQMNSLRAEDTAVYYCARGGNFYNYFDYWGQGTLVTVSS

SEQ ID NO: 137 - protein, 122 aa, synthetic construct
EVQLLESGGGLVQPGGSLRLSCAASGFTFSDHYMSWVRQAPGKGLEWVSTISSGGSYVYYADSVKGRFTISRDNSKNTLYLQMNSLRVEDAAVYYCAKILKNGKYIYYFDYWGQGTLVTVSS

SEQ ID NO: 138 - protein, 122 aa, synthetic construct
EVQLLESGGGLVQPGGSLRLSCAASGFTFSDHYMSWVRQAPGKGLEWVSTISSGGSYKYYADSAKGRFTISRDNSKNTLYLQMNSLRAEDTAVYYCAKILKNGNYIYYFDYWGQGTLVTVSS

SEQ ID NO: 139 - protein, 116 aa, synthetic construct
EVQLLESGGGLVQPGGSLRLSCAASGFTFSDYWMSWVRQAPGKGLEWVSSIYSGTSYIYYADSVKGRFTISRDNSRNTLYLQMNSLRAEDTAVYYCTRGWTYLDYWGQGTLVTVSS

SEQ ID NO: 140 - protein, 122 aa, synthetic construct
EVQLLESGGGLVQPGGSLRLSCAASGFTFSDHYMSWVRQAPGKGLEWVSTISSGGSYVYYADSVKGRFTISRDNSKNTLHLQMNSLRVEDAAVYYCAKILKNGKYIYYFDYWGQGTLVTVSS

SEQ ID NO: 141 - protein, 120 aa, synthetic construct
EVQLLESGGGLVQPGGSLRLSCAAFGFTFTGYDMTWVRQAPGKGLEWVSAISWNTGSTTYYADSVKGRFTISRDNSKNTLYLQMNSLRAEDTAVYYCTRGYDRKNYFEYWGQGTLVTVSS

SEQ ID NO: 142 - protein, 116 aa, synthetic construct
EVQLLESGGGLVQPGGSLRLSCAASGFTFSDYWMSWVRQAPGKGLEWVSSIYSGPSYIYYADSVKGRFTISRDNPKNTLYLQMNSLRAEDTAVYYCTRGWTYFDYWGQGTLVTVSS

SEQ ID NO: 143 - protein, 116 aa, synthetic construct
EVQLLESGGGLVQPGGSLRLSCAASGFTSSDYWMSWVRQTPGKGLEWVSSIYSGPSYIYYADSVKGRFTISRDNSKNTLYLQMNSLRAEDTAVYYCTRGWTYFDYWGQGTLVTVSS

SEQ ID NO: 144 - protein, 116 aa, synthetic construct
EVQLLESGGGLVQPGGSLRLSCAASGFTFSDYWMSWVRQTPGKGLEWVSSIYSGPSYIYYADSVKGRFTISRDNSKNTLYLQMNSLRAEDTAVYYCTRGWTYFDYWGQGTLVTVSS

SEQ ID NO: 145 - protein, 114 aa, synthetic construct
EVQLLESGGGLVQPGGSLRLSCVASGFTFSDYYMGWVRQAPGKGLEWVSAIGTIVTYYADSVKGRFTISRDNSKNTLYLQMNSLRADDTAVYYCARGINYVDDWGQGTLVTVSS

SEQ ID NO: 146 - protein, 115 aa, synthetic construct
EVQLLESGGGLVQPGGSLRLSCAASGFTFSSYWMSWVRQAPGKGLEWVSTIGSPGDTYYADSVKGRFTISRDNSKNTLYLQLNSLTAEDTAVYFCATGYAIFDYWGQGTLVTVSS

SEQ ID NO: 147 - protein, 114 aa, synthetic construct
EVQLLESGGGLVQPGGSLRLSCVASGFTFSDYYMSWVRQAPGKGLEWVSAIGTIITYYADSVKGRFTISRDNSKNTLYLQMNSLRADDTAVYYCARGINFVDDWGQGTLVTVSS

SEQ ID NO: 148 - protein, 119 aa, synthetic construct
EVQLLESGGGLVQPGGSLRLSCAASGFTFSGYDMSWVRQAPGKGLEWVSAIGWKSGSIYYADSVKGRFTISRDNSKNTLYLQMNSLRAEDTAVYYCAKGYNLKNYFDYWGQGTLVTVSS

SEQ ID NO: 149 - protein, 116 aa, synthetic construct
EVQLLESGGDSVQPGESLRLSCAASGFTFSSSAMHWVRQAPGKGLEWVSATNNDGSITYYADSVKGRFTISRDNSKNTLYLQMNGLRAEDTAVYYCARIIYYFDYWGQGTLVTVSS

SEQ ID NO: 150 - protein, 119 aa, synthetic construct
EVQLLESGGGLVQPGGSLRLSCAASGFIFSGYDMSWVRQAPGKGLEWVSAISWTSGSIYYADSVKGRFTISRDNSKNTLYLQMNSLRAEDTAVYYCAKGYNRNNYFDYWGQGTLVTVSS

SEQ ID NO: 151 - protein, 120 aa, synthetic construct
EVQLLESGGGLVQPGGSLRLSCAASGFTFSDYWMSWVRQAPGKGLEWVSSIYSGGSSSRSYIYYADSVKGRFTISRDNSKNTLYLQMSSLRAEDTAVYYCTRGWAYFDYWGQGTLVTVSS

SEQ ID NO: 152 - protein, 116 aa, synthetic construct
EVQLLESGGGLVQPGGSLRLSCAASGFTFSTYWMSWVRQAPGKGLEWVSNIYSGGSTIDYADSVKGRFTISRDNSKNTLYLQMNSLRAEDTAVYYCARGWSYFDYWGQGTLVTVSS

SEQ ID NO: 153 - protein, 119 aa, synthetic construct
EVQLLESGGGLVQPGGSLRLSCAASGFIFSGYDMSWVRQAPGKGPEWVSAITWDSGSTYYADSVKGRFTISRDNSKNTLYLQTNSLRAEDTAVYYCAKGYDRNNYFEYWGQGTLVTVSS

SEQ ID NO: 154 - protein, 115 aa, synthetic construct
EVQLLESGGGLIQPGGSLRLSCAASGFNFSDYWMSWVRQAPGKGLEWISAIYSGGYIYYADSVKGRFTISRDNSKNTLYLQMNSLRAEDTAVYYCTRGWSYCDYWGQGTLVTVSS

SEQ ID NO: 155 - protein, 115 aa, synthetic construct
EVQLLESGGGLVQPGGSLRLSCAASGFNFSDYWMSWVRQAPGKGLEWVSAIYSGGYIYYADSVKGRFTISRDNSKNTLYLQMNSLRAEDTAVYYCARGWSYCDSWGQGTLVTVSS

SEQ ID NO: 156 - protein, 122 aa, synthetic construct
EVQLLESGGGLVQPGGSLRLSCAASGFTFSDHYMSWVRQAPGKGLEWVSTISSGGNYIYYADSVKGRFTISRDSSKNTLYLQMNSLRAEDTAVYYCARILKNGKYIYYFDYWGQGTLVTVSS

SEQ ID NO: 157 - protein, 116 aa, synthetic construct
DVQLVESGGGVVRPGESLRLSCAASGFTFSSYDMSWVRQAPGEGLEWVSAISGSGVSAYYADSVKGRFTISRDNSKNTLYLQMNSLRAEDTAIYYCAKDGLFFDYWGLGTLVTVSS

SEQ ID NO: 158 - protein, 118 aa, synthetic construct
DVQLVESGGGVVRPGESLRLSCAASGFNFSSYDMNWVRQAPGEGLQWVATITAAGDITYYADSVRGRFAISRDNSKNTLYLGLSSLRAEDTAIYYCGREPWNSFSDYWGLGTLVTVSS

SEQ ID NO: 159 - protein, 115 aa, synthetic construct
DVQLVESGGGVARPGEFLRLSCAASGFTFRDYDMGWVRQAPGEGLEWVSSISVSGTTYYADSVKGRFTISRDNSENTLYLQMNSLTAEDTAVYYCGREDTLFHYWGLGTLVTVSS

SEQ ID NO: 160 - protein, 118 aa, synthetic construct
DVQLVESGGGVVRPGESLRLSCAASGFTFRNYDMTWVRQAPGEGLEWVATISGTDDTTYYADSVKGRATISRDNSNNTLYLRLFSLRAEDTAIYYCAREASNSFIDYWGLGTLVTVSS

SEQ ID NO: 161 - protein, 120 aa, synthetic construct
DVQLVESGGGVVRPGESLRLSCAASGFTFSNYDMTWVRQAPGEGLEWVSSISPSAYSAYYADSVKGRFTISRDNSKNTLYLEMNSLRAEDTAVYYCAKTSGNINYGLDYWGLGTLVTVSS

SEQ ID NO: 162 - protein, 115 aa, synthetic construct
DVQLVESGGGVARPGESLRLSCAASGFTFRDYDMGWVRQAPGEGLEWVSSISVSGTTYYADSVKGRFTISRDNSENTLYLQMNSLTAEDTAVYYCGREDTLFHYWGLGTLVTVSS

SEQ ID NO: 163 - protein, 120 aa, synthetic construct
EVQLLESGGDLLQPGGSLRLSCSASGFDFSSYYMTWVRQAPGKGLEWVAAITSLGYTTYYANSVEGRFTISRDNSENTLYLEMNSLRAEDTAVYYCATTHARGSRYFDYWGQGTLVTVSS

SEQ ID NO: 164 - protein, 120 aa, synthetic construct
EVQLLESGGGLLQPGGSLRLSCSASGFDFSSYYMTWVRQAPGKGLEWVAAITSLGYTTYYANSVEGRFTISRDNSENTLYLEMNSLRAEDTAVYYCATTHARGSRYFDYWGQGTLVTVSS

SEQ ID NO: 165 - protein, 121 aa, synthetic construct
EVQLLESGGGLVQPGGSLRLSCAASGFSFSDYHMSWVRQAPGKGLEWVSSISWNRGTTHYADSVRGRFTISRDNSKNTLYLQMNSLRAEDTAVYYCTRMQVYLMTSYFDYWGQGTLVTVSS

SEQ ID NO: 166 - protein, 121 aa, synthetic construct
EVQLLESGGGSVQPGGSLRLSCAASGFTFSDYYMSWVRQAPGKGLEWVSAISWNNGRTYYADSVKGRFTISRDDSKNTLYLQMNSLTAEDTAVYYCAKMLVYLMTSYFDSWGQGTLVTVSS

SEQ ID NO: 167 - protein, 121 aa, synthetic construct
EVQLLESGGGLVQPGGSLRLSCAASGFSFSDYHMSWVRQAPGKGLEWVSTISWNRGTTHYADSVKGRFTISRDNSKNTLYLQMNSLRAEDTAVYYCTRMQVYLMTSYFDYWGQGTLVTVSS

SEQ ID NO: 168 - protein, 121 aa, synthetic construct
EVQLLESGGGLVQPGGSLRLSCAASGFSFSDYHMSWVRQAPGKGLEWVSTISWNRGTTHYADSVKGRLTISRDNSKNTLYLQMNSLRAEDTAVYYCTRMQVYLMTSYFDYWGQGTLVTVSS

SEQ ID NO: 169 - protein, 121 aa, synthetic construct
EVQLLESGGGSVQPGGSLRLSCAASGFTFSDYYMSWVRQAPGKGLEWVSAISWNNGRMYYADSVKGRFTISRDDSKNTLYLQMNSLTAEDTAVYYCAKMLVYLMTSYFDSWGQGTLVTVSS

SEQ ID NO: 170 - protein, 119 aa, synthetic construct
EVQLLESGGGLVQPGGSLRLSCVGSGFVFDEYGIHWVRQAPGKGLEWVAAIDWSGNRTYYADSVKGRFTISRDNSKNTAYLQMNSLTAEDTAVYYCAKFRWRDFYFEYWGQGTLVTVSS

SEQ ID NO: 171 - protein, 121 aa, synthetic construct
EVQLLESGGGLVQPGGSLRLSCAASGFSFSDYHMSWVRQAPGKGLEWVSSISWNRGTTHYADSVKGRFTISRDNSKNTLYLQMSSLRAEDTAVYYCTRMQVYLMTSYFDYWGQGTLVTVSS

SEQ ID NO: 172 - protein, 119 aa, synthetic construct
EVQLLESGGGLVQPGGSLRLSCVGSGFVFDEYGIHWVRQAPGKGLEWVAAIDWSGNRTYYADSVKGRFTISRDNSKNTVYLQMNSLTAEDTAVYYCAKFRWRDFYFEYWGQGTLVTVSS

SEQ ID NO: 173 - protein, 119 aa, synthetic construct
EVQLLESGGGLVQPGGSLRLSCVGSGFVFDEYGIHWVRQAPGKGLEWVAAIDWSGNRTYYADSVKGRFTISRDNSRNTVYLQMNSLTAEDTAVYYCAKFRRREFYFEYWGQGTLVTVSS

SEQ ID NO: 174 - protein, 121 aa, synthetic construct
EVQLLESGGGLVQPGGSLRLSCAASGFSFSDYHMSWVRQAPGKGLEWVSSISWNRGTTHYADSVKGRFTISRDNSKNTLYLQMNSLRAEDTAVYYCTRMQVYLMTSYFDYWGQGTLVTVSS

SEQ ID NO: 175 - protein, 121 aa, synthetic construct
EVQLLESGGGLVQPGGSLRLSCAASGFTLSDYWMSWVRQAPGKGLEWVSDISWSAGDTYYYADSVKGRFTISRDNSKNTLYLQMNSLRAEDTAVYYCAKYQRNGGYSFDYWGQGTLVTVSS

SEQ ID NO: 176 - protein, 121 aa, synthetic construct
EVQLLESGGGLVQPGGSLRLSCAASGFTFDDYGMSWVRQAPGKGLEWVSAISWSGGRTYYADSVKGRFTISRDDSKNTLYLQMNSLRAEDTAVYYCTRDITILTTYYFDYWGQGTLVTVSS

SEQ ID NO: 177 - protein, 117 aa, synthetic construct
EVQLLESGGGLVQPGGSLRLSCAASGFMFNGSIMQWVRQAPGKGLEWVAGISDNGGTSYPDFVKGRFTISRDNSKNTVYLQMNSLRAEDTAVYYCVKDIDGYYFDYWGQGTLVTVSS

SEQ ID NO: 178 - protein, 120 aa, synthetic construct
EVQLLESGGGLVQPGGSLRLSCAVSGFTFSGSAMSWVRQAPGKGREYVAGISSNGGTTYFTDSMKGRFTISRDNSKNTLYLQMSSLRAEDTAVYYCARGGYNWNIYIDYWGQGTLVTVSS

SEQ ID NO: 179 - protein, 118 aa, synthetic construct
EVQLLESGGAFVQPGRSLGLSCAASGFTFSDYYMSWVRQAPGKGLEWVSVISNSGGSTYYTDSVKGRFTISRDNSKNTLYLQMNSLRAEDTAVYYCTKDIGMTYFDYWGQGTLVTVSS

SEQ ID NO: 180 - protein, 118 aa, synthetic construct
EVQLLESGGALVQPGGSLRLSCAASGFTFSDYYMSWVRQAPGKGLEWVSVISNSGGSTYYADSVKGRFTISRDNSKNTLYLQMNSLRAEDTAVYYCTKDIGMTYFDYWGQGTLVTVSS

SEQ ID NO: 181 - protein, 119 aa, synthetic construct
EVQLLESGGGLVQPGGSLRLSCAASGFDFSGSPMTWVRQAPGKGLEWVSVIRSKANSYATYYADSVKGRFTISRDNSKNTLYLQMNSLRAEDTAVYYCARGGTGYFDYWGQGTLVTVSS

SEQ ID NO: 182 - protein, 120 aa, synthetic construct
EVQLLESGGGLVQPGGSLRLSCAASGFTFSGSALSWVRQAPGKGLEWVSAIFSNGGSAYYADSVKGRFTISRDNSKNTLYLQMNSLRAEDTAVYYCAKGGYNWNNYLEYWGQGTLVTVSS

SEQ ID NO: 183 - protein, 118 aa, synthetic construct
EVQLLESGGAFVQPGGSLRLSCAASGFTFSDYYMSWVRQAPGKGLEWVSVISNSGGSTYYTDSVKGRFTISRDNSKNTLYLQMNSLRAEDTAVYYCTKDIGMTYFDYWGQGTLVTVSS

SEQ ID NO: 184 - protein, 118 aa, synthetic construct
EVQLLESGGGLVQPGGSLRLSCAASGFTFSNYEMNWVRQAPGKGLEWVGIIGTGGAITYYADSVKGRFTISRDNSKNTLYLQMNSLRAEDTAIYYCVKYSTESYFDYWGQGTLVTVSS

SEQ ID NO: 185 - protein, 123 aa, synthetic construct
EVQLLESGGGLVQPGGSLRLSCAASGFDFSGYDMTWVRQAPGKGLEWVSSITWNSWIDGTKIYYADSVKGRFTISRDNSNNTLYLQMNSLRDEDTAIYYCAGGSLTVNYFDYWGQGTLVTVSS

SEQ ID NO: 186 - protein, 114 aa, synthetic construct
EVQLLESGGGLVQPGGSLRLSCAASGFTFSDYWMTWVRQAPGKGLEWVSTISDSSNGGRTYYADSVKGRFTISRDNSKNTLYLQMNSLRAEDTAVYYCTKFDSWGQGALVTVSS

SEQ ID NO: 187 - protein, 118 aa, synthetic construct
EVQLLESGGAFVQPGGSLRLSCAASGFTFSDYYMSWVRQAPGKGLEWVSVISNSGGSTYYTDSVKGRFTISRDNSKNTLYLQINSLRAEDTAVYYCTKDIGMTYFDYWGQGTLVTVSS

SEQ ID NO: 188 - protein, 120 aa, synthetic construct
EVQLLESGGGLVQPGGSLRLSCAASGFTFSGSALSWVRQAPGKGLEWVSAIFSNGGSAYYADSVKGRFTISRDNSKNTLYLQMNSLRAEDTAVYYCAEGGYNWNNYLEYWGQGTLVTVSS

SEQ ID NO: 189 - protein, 118 aa, synthetic construct
EVQLLESGGGLVQPGGSLRLSCAASGFTFSDYDMAWVRQAPGKGLEWVSTITNTGSHIYYADSVKGRSTISRDNSENTLYLQMNSLRAEDTAVYYCVKEGTITIFDYWGQGTLVTVSS

SEQ ID NO: 190 - protein, 116 aa, synthetic construct
EVQLLESGGGLVQPGGSLRLSCAASGFTFSDYWMSWVRQAPGKGLEWVSSIYSGGSYIYYADSVKGRFTISRDNSKNTLYLQMSSLRAEDTAVYYCTRGWAYFDYWGQGTLVTVSS

SEQ ID NO: 191 - protein, 116 aa, synthetic construct
EVQLLESGGGLVQPGGSLRLSCAASGFTFSDYWMSWVRQAPGKGLEWVSSIYSGPSYIYYADSVKGRFTISRDNSKNTLYLQMNSLGAEDTAVYYCTRGWTYSDYWGQGTLVTVSS

SEQ ID NO: 192 - protein, 116 aa, synthetic construct
EVQLLESGGGLVQPGGSLRLSCAASGFTFSDYWMSWVRQAPGKGLEWVSSIYSGPSYIYYADSVKGRFTISRDNSKNTLYLQMNSLRAEDTAVYYCTRGWTYFDYWDQGTLVTVSS

SEQ ID NO: 193 - protein, 119 aa, synthetic construct
EVQLLESGGGLVQPGGSLRLSCAASGFIFSGYDMSWVRQAPGKGLEWVSAIIWNSNTIYYADSVKGRFTISRDNSKNTVYLQMNSLRAEDTAVYYCARGYNLKNYFDYWGQGTLVTVSS

SEQ ID NO: 194 - protein, 116 aa, synthetic construct
EVQLLESGGGLVQPGGSLRLSCAASGFTFSDYWMSWVRQAPGKGLEWVSSIYSGGSYIYYADSVKGRFTISRDNSENTLYLQMSSLRAEDTAVYYCTRGWAYFDYWGQGTLVTVSS

SEQ ID NO: 195 - protein, 119 aa, synthetic construct
EVQLLESGGGLVQPGGSLRLSCAASGFIFSGYDMSWVRQAPGKGLEWVSAIIWNSNTIYYADSVKGRFTISRDNSKNTVYLQMNSLRAEDTAVYYCVRGYNLKNYFDYWGQGTLVTVSS

SEQ ID NO: 196 - protein, 122 aa, synthetic construct
EVQLLESGGGLVQPGGVLRLSCTASGFTFDDYGMHWVRQAPEKGLEWVGAINWNGNVTHYADSVKGRFTISRDNSKNTLYLRMNSLRAEDTAVYYCAKNSGSEKRNYYFGYWGQGTLVTVSS

SEQ ID NO: 197 - protein, 116 aa, synthetic construct
EVQLLESGGGLVQPGGSLRLSCAASGFTFGDYWMSWVRQAPGKGLEWVSTIYSGVATIYYADSVKGRFTISRDNSKNTLYLQMNSLRAEDTAVYYCARGWTYFDYWGQGTLVTVSS

SEQ ID NO: 198 - protein, 122 aa, synthetic construct
EVQLLESGGGLVQPGGSLRLSCTASGFTFDDYGMHWVRQAPEKGLEWVGAINWNGNVTHYADSVKGRFTISRDNSKNTLYLRMNSLRAEDTAVYYCAKNSGSEKRNYYFGYWGQGTLVTVSS

SEQ ID NO: 199 - protein, 117 aa, synthetic construct
EVQLLESGGGLVQPGGPLRLSCAASGFTFSDYWMSWVRQAPGKGLEWVSSIYTGGSSYIYYADSVKGRFTISRDNSKNTLYLQMNSLRAEDTAVYYCTRGWTYFDYWGQGTLVTVSS

SEQ ID NO: 200 - protein, 122 aa, synthetic construct
EVQLLESGGGLVQPGGSLRLSCTASGFTFDDYGMHWVRQAPEKGLEWVGAVNWNGNVTHYADSVKGRFTISRDNSKNTLYLRMNSLRAEDTAVYYCAKNSGSEKRNYYFGYWGQGTLVTVSS

SEQ ID NO: 201 - protein, 115 aa, synthetic construct
EVQLLESGGGLVQPGGSLRLSCAASGFTFDDYWMSWVRQAPGKGLEWVSAIYSGSYIYYADSVKGRFTISRDNSKNTLYLQMNSLRAEDTAVYYCARGWSYFDYWGQGTLVTVSS

SEQ ID NO: 202 - protein, 116 aa, synthetic construct
EVQLLESGGGLVQPGGSLRLSCAASGFTFSDYWMSWVRQAPGKGLEWVSSIYSGTTYIYYADSVKGRFTISRDNSENTLYLQMNSLRAEDTAVYYCTRGWTYFDYRGQGTLVTVSS

SEQ ID NO: 203 - protein, 116 aa, synthetic construct
EVQLLESGGGLIQPGGSLRLSCAASGFTFSDYWMSWVRQAPGKGLEWVSSIYSGSSYIYYADSVKGRFTISRDNSKNTLYLQMNSLRAEDTAVYYCTRGWTYFDYWGQGTLVTVSS

SEQ ID NO: 204 - protein, 116 aa, synthetic construct
EVQLLESGGGLVQPGGSLRLSCAASGFTFSDYWMSWVRQAPGKGLEWVSSIYSGVSYIYYADSVKGRFTISRDNSKNTLYLQMNSLRAEDTAVYYCTRGWTYFDYWGQGTLVTVSS

SEQ ID NO: 205 - protein, 116 aa, synthetic construct
EVQLLESGGGLVQPGGSLRLSCAASGFTFSGYWMSWVRQAPGKGLEWVSSIYSGSSYIYYADSVKGRFTISRDKSKNTLYLQMNSLRAEDTAVYYCTRGWTYFDYWGQGTLVTVSS

SEQ ID NO: 206 - protein, 116 aa, synthetic construct
EVQLLESGGGLVQPGGSLRLSCAASGFTFSDYWMSWVRQAPGKGLEWVSSIYSGPSYIYYADSVKGRFTISRDNSKNTLYLQMNSLRAEDTAVYYCTRGWTYFDYLGQGTLVTVSS

SEQ ID NO: 207 - protein, 117 aa, synthetic construct
EVQLLESGGGLVQPGGSLRLSCAASGFTFSDYWMSWVRQAPGKGLEWVSSIYTGGSSYIYYADSVKGRFTISRDNSKNTLYLQMNSLRAEDTAVYYCTRGWTYFDYWGQGTLVTVSS

SEQ ID NO: 208 - protein, 116 aa, synthetic construct
EVQLLESGGGLVQPGGSLRLSCAASGFTFSDYWMSWVRQAPGKGLEWVSSIYSGATYIYYADSVKGRFTISRDNSKNTLYLQMNSLRAEDTAVYYCTRGWTYFDYWGQGTLVTVSS

SEQ ID NO: 209 - protein, 116 aa, synthetic construct
EVQLLESGGGLVQPGGSLRLSCAASGFTFSDYWMSWVRQAPGKGLEWVSSIYTGGSYIYYADSVKGRFTISRDNSKNTLYLQMNSLRAEDTAVYYCTRGWTYFDYWGQGTLVTVSS

SEQ ID NO: 210 - protein, 116 aa, synthetic construct
EVQLLESGGGLVQPGESLRLSCAASGFTFSDYWMSWVRQAPGKGLEWVSSIYSGPSYIYYADSVKGRFTISRDNSKNTLYLQMNSLRAEDTAVYHCTRGWTYFDYWGQGTLVTVSS

SEQ ID NO: 211 - protein, 116 aa, synthetic construct
EVQLLESGGGLVQPGGSLRLSCAASGFTFSDYWMSWVRQAPGKGLEWVSSIYSGPTYIYYADSVKGRFTISRDNSKNTLYLQMNSLRAEDTAVYYCTRGWTYFDYWGQGTLVTVSS

SEQ ID NO: 212 - protein, 119 aa, synthetic construct
EVQLLESGGGLVQPGGSLRLSCAASGFIFSGYDMSWVRQAPGKGLEWVSAIIWNSNTIYYADSVKGRFTISRDNSKNTVYLRMNSLRAEDTAVYYCARGYNLKNYFDYWGQGTLVTVSS

SEQ ID NO: 213 - protein, 116 aa, synthetic construct
EVQLLESGGGLVQPGGSLRLSCAAPGFTFSDYWMSWVRQAPGKGLEWVSSIYSGGSYIYYADSVKGRFTISRDNSKNTLYLQMSSLRAEDTAVYYCTRGWAYFDYWGQGTLVTVSS

SEQ ID NO: 214 - protein, 116 aa, synthetic construct
EVQLLESGGGLVQPGGSLRLSCAASGFTFSDYWMSWVRQAPGKGLEWVSSIYSGGPYIYYADSVKGRFTISRDNSKNTLYLQMNSLRAEDTAVYYCTRGWTYFDYWGQGTLVTVSS

SEQ ID NO: 215 - protein, 116 aa, synthetic construct
EVQLLESGGGLVQPGGSLRLSCAASGFTFDDYWMSWVRQAPGKGLEWVSTIYSGVATIYYADSVKGRFTISRDNSKNTLYLQMNSLRAEDTAVYYCARGWTYFDYWGQGTLVTVSS

SEQ ID NO: 216 - protein, 116 aa, synthetic construct
EVQLLEFGGGLVQPGGSLRLSCAASGFTFDDYWMSWVRQAPGKGLEWVSTIYSGVATIYYADSVKGRFTISRDNSKNTLYLQMNSLRAEDTAVYYCARGWTYFDYWGQGTLVTVSS

SEQ ID NO: 217 - protein, 115 aa, synthetic construct
EVQLLESGGGLVQPGGSLRLSCAASGFTFSDYWMSWVRQAPGKGLEWVSTIYSVGTIYYADSVKGRFTISRDNSKNTLYLQMDSLRAEDTAVYYCARGWTYFDYWGQGTLVTVSS

SEQ ID NO: 218 - protein, 119 aa, synthetic construct
EVQLLESGGGLVQPGGSLRLSCAASGFIFSGYDMSWVRQAPGKGLEWVSAIGWKSGSIYYADSVKGRFTISRDNSKNTLYLQMNSLRAEDTAVYYCAKGYNLKNYFDYWGQGTLVTVSS

SEQ ID NO: 219 - protein, 116 aa, synthetic construct
EVQLLESGGGLVQPGGSLRLPCAASGFTFSDYWMSWVRQAPGKGLEWVSSIYSGVSYIYYADSVKGRFTISRDNSKNTLYLQMNSLRAEDTAVYYCTRGWTYFDYWGQGTLVTVSS

SEQ ID NO: 220 - protein, 116 aa, synthetic construct
EVQLLESGGGLVQPGGSLRLSCAASGFTFSDYWMSWVRQAPGKGLEWVSSIYSGPSYIYYADSVKGRFTISRDNSKNTLYLQMNSPRAEDTAVYYCTRGWTYFDYWGQGTLVTVSS

SEQ ID NO: 221 - protein, 119 aa, synthetic construct
EVQLLESGGGLVQPGGSLRLSCAASGFIFSGYDMSWVRQAPGKGLEWVSAISWTSDSIYYADSVKGRFTISRDNFENTLYLQMNSLRAEDTAVYYCAKGYNRNNYFDYWGQGTLVTVSS

SEQ ID NO: 222 - protein, 116 aa, synthetic construct
EVQLLESGGGLVQPGGSLRLSCAASGFTFSDYWMSWVRQAPGKGLEWVSFIYSGPSYIYYADSVKGRFTISRDNSKNTLYLQMNSLRAEDTAVYYCTRGWTYFDYWGQGTLVTVSS

SEQ ID NO: 223 - protein, 116 aa, synthetic construct
EVQLLESGGGLVQPGGSLRLSCAASGFTFSDYWMSWVRQASGKGLEWVSSIYSGGSYIYYADSVKGRFTISRDNSKNTLYLQMSSLRAEDTAVYYCTRGWAYFDYWGQGTLVTVSS

SEQ ID NO: 224 - protein, 116 aa, synthetic construct
EVQLLESGGGLVQPGGSLRLSCAASGFTFSDYWMSWVRQAPGKGLEWVSSIYSGASYIYYADSVKGRFTISRDNSKNTLYLQMNSLRAEDTAVYYCTRGWTYFDYWGQGTLVTVSS

SEQ ID NO: 225 - protein, 116 aa, synthetic construct
EVQLLESGGGLVQPGGSLRLSCAASGFTFSAYWMSWVRQAPGKGLEWVSSIYSGTTYIYYADSVKGRFTISRDNSENTLYLQMNSLRAEDTAVYYCTRGWTYFDYWGQGTLVTVSS

SEQ ID NO: 226 - protein, 118 aa, synthetic construct
EVQLLESGGGLVQPGGSLRLSCAASGFTFNDYWMSWVRQAPGKELEWVSAIYSGGSSSYIYYADSVKGRFTISRDNSKNTLYLQMNSLRAEDTAVYYCARGWSYFDYWGQGTLVTVSS

SEQ ID NO: 227 - protein, 115 aa, synthetic construct
EVQLLESGGGSVQPGGSLRLSCAASGFAFSSYWMSWVRQAPGKGLEWVSAMTGTSYIYYADSVKGRFTISRDNSKNTLYLQMNSLRAEDTAVYYCARGWAYLDYWGQGTLVTVSS

SEQ ID NO: 228 - protein, 119 aa, synthetic construct
EVQLLESGGGLVQPGGSLRLSCAASGFIFSGYDMSWVRQAPGKGLEWVSAISWTSGSIYYADSVKGRFTISRDNSLNTLYLQMNSLRAEDTAVYYCAKGYNRNNYFDYWGQGTLVTVSS

SEQ ID NO: 229 - protein, 116 aa, synthetic construct
DVQLVESGGGVVRPGESLRLSCAASGFTFSNFYMNWVRQAPGEGLEWVSTISGIDDTTWYADSVKGRFAISRDNSRNTLYLQMNSLRAEDTATYYCAKGTDFLDYWGLGTLVTVSS

SEQ ID NO: 230 - protein, 118 aa, synthetic construct
DVQLVESGGGVVRPGESLRLSCAASGFTFSDYDMAWVRQAPGEGLEWVATISGTDDSTYYADSVKGRATISRDNSNNTLYLRLFSLRAEDTAIYYCAREASNSFIDYWGLGTLVTVSS

SEQ ID NO: 231 - protein, 117 aa, synthetic construct
DVQLVESGGGVVRPGESLRLSCIASGFTFTTYDMSWVRQAPGEGLEWVSAIRPSGSVTYYADSVKGRFTISRDNSKNTLYLQMNSLRGEDTAIYYCATEASYSVFDWGLGTLVTVSS

SEQ ID NO: 232 - protein, 118 aa, synthetic construct
DVQLVESGGGLVQPGGSLRLSCAASGFTFNSYDMNWVRQAPGEGLEWVSTISGDGGDIYYADSVKGRFTISRDNSKNTLYLQMNSLRAEDTAIYYCGREGLNAFSDYWGLGTLVTVSS

SEQ ID NO: 233 - protein, 118 aa, synthetic construct
DVQLVESGGGVVRPGESLRLSCAASGFTFSNYDMTWVRQAPGEGLEWVATISGTDDSTYYADSVKGRATIFRDNSNNTLYLRLLSLRAEDTAIYYCAREASNSFIDYWGLGTLVTVSS

SEQ ID NO: 234 - protein, 117 aa, synthetic construct
DVQLVESGGGVVRPGESLRLSCIASGFTFSTNDMSWVRQAPGEGLEWVSAIRPSGSVTYYADSVKGRFTISRDNSKNTLYLQMNSLRGEDTAIYYCAREASYSVFDWGLGTLVTVSS

SEQ ID NO: 235 - protein, 118 aa, synthetic construct
DVQLVESGGGVVRPGESLRLSCAASGFTFSNYDMTWVRQAPGEGLEWVATISGTDYTTYYAASVKGRATISRDNSNNTLYLRLFSLRAEDTAIYYCTREASNSFIDYWGLGTLVTVSS

SEQ ID NO: 236 - protein, 118 aa, synthetic construct
DVQLVESGGGVVRPGESLRLSCTASGFSFYNYAMNWVRQAPGEGLEFVADISGSGDTTSYAPAVKGRFTISRDNSKNTLYLQMNSLRAEDTAIYYCAKDSGDILFDYWGLGTLVTVSS

SEQ ID NO: 237 - protein, 118 aa, synthetic construct
DVQLVESGGGVVRPGESLRLSCAASGFTFSNYDMTWVRQAPGEGLEWVATISGIDDSTYYADSVKGRATISRDNSNNTLYLRLFSLRAEDTAIYYCAREASNSFIDYWGLGTLVTVSS

SEQ ID NO: 238 - protein, 118 aa, synthetic construct
DVQLVESGGGVVRPGESLRLSCAASGFTFSSYAMNWVRQAPGEGLEYVADISGSGDATGYADSVKGRFTISRDNSKNTLYLQMNSLRAEDTAIYYCAKDSGDIFFDYWGLGTLVTVSS

SEQ ID NO: 239 - protein, 118 aa, synthetic construct
DVQLVESGGGVVRPGESLRLSCAASGFTFRNYDMAWVRQAPGEGLEWVATISGTDGTTYYADSVKGRATISRDNSNNTLYLRLLSLRDEDTAIYYCAREASNSFIDYWGLGTLVTVSS

SEQ ID NO: 240 - protein, 118 aa, synthetic construct
DVQLVESGGGVVRPGESLRLSCAASGFTFRNYDMTWVRQAPGEGLEWVATISGTDDTTYYADSVKGRATISRDNSNNTLYLRLFSLRAEDTAIYYCAREASNSFIDYWGPGTLVTVSS

SEQ ID NO: 241 - protein, 118 aa, synthetic construct
DVQLVESGGGVVRPGDSLTLSCAASGFTFSNYAMSWVRQAPGEGLEWVASISTNGGSTGYADSVKGRFTISRDNSKNTLYLRLFSLRAEDTAIYYCTKETWNTSFDYWGLGTLVTVSS

SEQ ID NO: 242 - protein, 118 aa, synthetic construct
DVQLVESGGGVVRPGESLRLSCAASGFTFSNYDMTWVRQAPGEGLEWVTTISGTDDSTYYADSVKGRATIFRDNSNNTLYLRLLSLRAEDTAIYYCAREASNSFIDYWGLGTLVTVSS

SEQ ID NO: 243 - protein, 118 aa, synthetic construct
DVQLVESGGGVVRPGESLRLSCAASGFTFSNYDMTWVRQAPGEGLEWVATISGTDYTTYYAASVKGRATISRDNSNNTLYLRLFSLRAEDTAIYYCAREASNSFIDYWGLGTLVTVSS

SEQ ID NO: 244 - protein, 116 aa, synthetic construct
DVQLVESGGGVVRPGESLRLSCAASGFTFSNFYMNWVRQAPGEGLEWVSTISGIDDTTWYADSVKGRFTISRDNSRNTLYLQMNSLRAEDTATYYCAKGTDFLDYWGLGTLVTVSS

SEQ ID NO: 245 - protein, 124 aa, synthetic construct
DVQLVESGGGVVRPGESLRLSCAASGFDFSNYDMNWVRQAPGEGLEWVATISGSASITGTANITYYAPAVKGRFTISRDNSKNTLYLRLFSLRAEDTAIYYCARETFDGFFDYCGLGTLVTVSS

SEQ ID NO: 246 - protein, 118 aa, synthetic construct
DVQLVESGGGVVRPGESLRLSCAASGFTFRNYDMTWVRQAPGEGLEWVATISGTDDTTYYADSVKGRAAISRDNSNNTLYLRLLSLRAEDTAIYYCAREASNSFIDYWGLGTLVTVSS

SEQ ID NO: 247 - protein, 118 aa, synthetic construct
DVQLVESGGGMVRPGESLRVSCAASGFDFSNYHMNWVRQAPGEGLEWVSRISDGDSRTYYSDFVKGRATISRDNSNNTLYLRLFSLRADDTAIYYCVRETLNNVFDYWGLGTLVTVSS

SEQ ID NO: 248 - protein, 116 aa, synthetic construct
DVQLVESGGGVVRPGESLRLSCAASGFTFSSHGMNWLRQAPGEGLESVAGIRSDGKYIYYADSVKGRATISRDDSKSTVYLQMDSLRAEDTAIYYCARGWNFFDYWGPGALVTVSS

SEQ ID NO: 249 - protein, 118 aa, synthetic construct
DVQLVDSGGGVVRPGESLRLSCAASGFTFRNYDMAWVRQAPGEGLEWVATISGTDGTTYYADSVKGRATISRDNSNNTLYLRLLSLRDEDTAIYYCAREASNSFIDYWGLGTLVTVSS

SEQ ID NO: 250 - protein, 118 aa, synthetic construct
DVQSVESGGGVVRPGESLRLSCAASGFNFSSYDMNWVRQAPGEGLQWVATITAAGDITYYADSVRGRFTISRDNSKNTLYLGLSSLRAEDTAIYYCGREPWNSFSDYWGLGTLVTVSS

SEQ ID NO: 251 - protein, 118 aa, synthetic construct
DVQLVESGGGVVRPGESLRLSCAASGFNCSSYDMNWVRQAPGEGLQWVATITAAGDITYYADSVRGRFAISRDNSKNTLYLGLSSLRAEDTAIYYCGREPWNSFSDYWGLGTLVTVSS

SEQ ID NO: 252 - protein, 118 aa, synthetic construct
DVQLVESGGGVVRLGESLRLSCAASGFNFIDYDMAWVRQAPGEGLEWVATISGTDDSTYYADSVKGRATISRDNSNNTLYLRLFSLRAEDTAIYYCAREASNSFIGYWGLGTLVTVSS

SEQ ID NO: 253 - protein, 118 aa, synthetic construct
DVQLVESGGGVVRPGESLRLSCAASGFTFRNYDMTWVRQAPGEGLEWVATISATDDTTYYADSVKGRATISRDNSNNTLYLRLFNLRAEDTAIYYCAREASNSFIDYWGLGTLVTVSS

SEQ ID NO: 254 - protein, 118 aa, synthetic construct
DVQLVESGGGVVRPGESLRLSCAASGFNFSSYDMNWVRQAPGEGPQWVATITAAGDITYYADSVRGRFAISRDNSKNTLYLGLSSLRAEDTAIYYCGREPWNSFSDYWGLGTLVTVSS

SEQ ID NO: 255 - protein, 118 aa, synthetic construct
DVQLVESGGGVVRPGESLRLSCAASGFDFINYVMNWVRQAPGEGLEFVARITNSGDRTWYADSVKGRFTISRDNSNNTLYLRLFSLRAEDTAIYYCVRETSNYLLDYWGLGTLVTVSS

SEQ ID NO: 256 - protein, 118 aa, synthetic construct
DVQLVESGGGVVRPGESLGLSCAASGFTFRNYDMTWVRQAPGEGLGWVATISGTDDTTYYADSVKGRAAISRDNSNNTLYLRLLSLRAEDTAIYYCAREASNSFIDYWGLGTLVTVSS

SEQ ID NO: 257 - protein, 118 aa, synthetic construct
DVQLVESGGGVVRPGESLRLSCAASGFNFSSYDMNWVRQAPGEGLQWVATITAAGDITYYADSVRGRFAISRDNSKNTLYLGLSSLRAKDTAIYYCGREPWNSFSDYWGLGTLVTVSS

SEQ ID NO: 258 - protein, 118 aa, synthetic construct
DVQLVESGGGVVRPGDSLRLSCAASGFTFSDYDMTWVRQAPGEGLEWVSTISGSGDRIYYADSVKGRFTISRDNSKNTLYLRLFSLRAEDTAIYYCAKEGWNSFIDYWGLGTLVTVSS

SEQ ID NO: 259 - protein, 118 aa, synthetic construct
DVQLVESGGGVVRPGESLRLSCAASGSNFSSYDMNWVRQAPGEGLQWVATITAAGDITYYADSVRGRFAISRDNSKNTLYLGLSSLRAEDTAIYYCGREPWNSFSDYWGLGTLVTVSS

SEQ ID NO: 260 - protein, 118 aa, synthetic construct
DVQLVESGGGVVRPGESLRLSCAASGFTFSNYDMTWVRQAPGEGLEWVATISGTDYTTYYAASVKGRATISRDNSNNTLYLRLFSLRAEDTAIYYCTREAPNSFIDYWGLGTLVTVSS

SEQ ID NO: 261 - protein, 118 aa, synthetic construct
DVQLVESGGGVVRPGESLRLSCAASGFDFINYVMNWVRQAPGEGLEFVARITNSGDRTWYADSVKGRFAISRDNSNNTLYLRLFSLRAEDTAIYYCVRETSNYLLDYWGLGTLVTVSS

SEQ ID NO: 262 - protein, 118 aa, synthetic construct
DVQLVESGGGVVRPGESLRLSCAASGFNFIDYDMAWVRQAPGEGLEWVATISGTDDSTYYADSVKGRATISRDNSNNTLYLRLFSLRAEDTAIYYCAREASNSFIDYWGLGTLVTVSS

SEQ ID NO: 263 - protein, 118 aa, synthetic construct
DVQLVESGGGVVRPGESLRLSCAASGFNFSSYDMNWVRQAPGEGLQWVATITAAGDITYYAGSVRGRFAISRDNSKNTLYLGLSSLRAEDTAIYYCGREPWNSFSDYWGLGTLVTVSS

SEQ ID NO: 264 - protein, 116 aa, synthetic construct
DVQLVESGGGVVRPGESLRLSCVASGFTFGSDGMNWVRQAPGKGLDWISTISIDGTPTYYAESVKGRFTISRDNSKNTLYLQMNSLRAEDTAIYYCVKGYNFFDYWGLGTLVTVSS

SEQ ID NO: 265 - protein, 118 aa, synthetic construct
DVQLVESGGGVVRPGESLRLSCAASGFTFSDYDMAWVRQAPGEGLEWVATISGTDDSTCYADSVKGRATISRDNSNNTLYLRLFSLRAEDTAIYYCAREASNSFIDYWGLGTLVTVSS

SEQ ID NO: 266 - protein, 117 aa, synthetic construct
DVQLVESGGGVVRPGESLRLSCIASGFTFSSYDIEWVRQAPGKGLEWVAAIDDDGSRRWYADSVKGRATISRDNSESTVYLQLNSLRAEDTAVYYCTRATLYSSYDWGLGTLVTVSS

SEQ ID NO: 267 - protein, 116 aa, synthetic construct
DVQLVESGGGLVQPGGSLRLSCAASGFTFSDFAMGWVRQAPGEGLEWVSTISGSGVITFYADSVKGRFTISRDNSKNTLYLQMNSLRVEDTAVYFCSEGMSYHDYWGLGTLVTVSS

SEQ ID NO: 268 - protein, 116 aa, synthetic construct
DVQLVESGGGVVQPGGSLRLSCAASGFTFSDFAMSWVRQAPGEGLEWVSTISGSGVVTFYADSVKGRFTISRDNSKNALYLQMNSLRVEDTAVYFCSEGMSYHDYWGLGTLVTVSS

SEQ ID NO: 269 - protein, 116 aa, synthetic construct
DVQLVESGGGLVQPGGFLRLSCAASGFTFRDFAMSWVRQAPGEGLEWVSSISHSGVITFYADSVKGRFTISRDNSKNTLYLQMNSLRVEDTAIYYCSEGLSYHDYWGLGTLVTVSS

SEQ ID NO: 270 - protein, 116 aa, synthetic construct
DVQLVESGGGVVQPGGSLRLSCAASGFTFRDFAMSWVRQAPGEGLEWVSSISHSGVITFYADSVKGRFTISRDNSKNTLYLQMNSLRVEDTAVYFCSEGLSYHDYWGLGTLVTVSS

SEQ ID NO: 271 - protein, 116 aa, synthetic construct
DVQLVESGGGVVRPGESLRLSCAASGFTFSSDAMSWVRQAPGEGLEWVSAISGSGVITYYADSVKGRFTISRDNSNNTLYLQMNSLRAEDTAIYYCATGFPFFDYWGLGTLVTVSS

SEQ ID NO: 272 - protein, 116 aa, synthetic construct
DVQLVESGGGVVRPGESLRLSCAASGFSFSSYDMSWVRQAPGEGLEWVSSISGSGAITYYADSVKGRFTISRDNSKNTLYLQMNSLRAEDTAVYYCAESMIFFDYWGLGTLVTVSS

SEQ ID NO: 273 - protein, 116 aa, synthetic construct
DVQLVESGGGLVQPGGSLRLSCAASGFTFSDFAMSWVRQAPGEGLEWVSTISGSGVIIFYADSVKGRFTISRDNSKNTLYLQMNSLRVEDTAVYFCSEGMSYHDYWGLGTLVTVSS

SEQ ID NO: 274 - protein, 117 aa, synthetic construct
DVQLVESGGGVVRPGESLRLSCAASGFSFDIYDMSWARQAPGKGLEWVSLISSSGVITYYADSVKGRFTISRDNSKSTLYLQMNSLRAEDTAVYYCARAGNTFFHYWGLGTLVTVSS

SEQ ID NO: 275 - protein, 116 aa, synthetic construct
DVQLVESGGGLVQPGGSLRLSCAASGFTFSSYAMSWVRQAPGEGLEWVSSISHSGVITFYADSVKGRFTISRDNSKNTLYLQMNSLRVEDTAVYYCSEGLSYHDYWGLGTLVTVSS

SEQ ID NO: 276 - protein, 117 aa, synthetic construct
DVQLVESGGGVVRPGESLRLSCAASGFSFDIYDMSWVRQAPGKGLEWVSLISSSGVITYYADSVKGRFTISRDNSKSTLYLQMNSLRAEDTAIYYCARAGNTFFHYWGLGTLVTVSS

SEQ ID NO: 277 - protein, 116 aa, synthetic construct
DVQLVESGGGLVQPGGSLRLSCAASGFTFSDFAMSWVRQAPGEGLEWVSSISHSGVITFYADSVKGRFTISRDNSKNTLYLQMNSLRVEDTAIYFCSEGLSYHDYWGLGTLVTVSS

SEQ ID NO: 278 - protein, 116 aa, synthetic construct
DVQLVESGGGVVRPGESLRLSCVASGFTFSTDTMSWVRQAPGEGLEWVSASSGSGVITYYADSAKGRFTISRDNSNNTLYLQMNSLRAEDTAIYYCATGFPFFDYWGLGTLVTVSS

SEQ ID NO: 279 - protein, 116 aa, synthetic construct
DVQLVESGGGLVQPGGSLRLSCAASGFTFSDFAMSWIRQAPGEGLEWVSTISGSGVITFYADSVKGRFTISRDNSKNTLYLQMNSLRVEDTAVYFCSEGMSYHDYWGLGTLVTVSS

SEQ ID NO: 280 - protein, 116 aa, synthetic construct
DVQLVESGGGLVQPGGSLRLSCAASGFTFSDFAVSWVRQAPGEGLEWVSTISGSGVITFYADSVKGRFTISRDNSKNTLYLQMNSLRVEDTAVYFCSEGMSYHDYWGLGTLVTVSS

SEQ ID NO: 281 - protein, 116 aa, synthetic construct
DVQLVESGGGVVRPGESPRLSCVASGFTFSTDTMSWVRQAPGEGLEWVSASSGSGVITYYADSVKGRFTISRDNSNNTLYLQMNSLRAEDTAIYYCATGFPFFDYWGLGTLVTVSS

SEQ ID NO: 282 - protein, 116 aa, synthetic construct
DVQLVESGGGVVRPGESLRLSCAASGFTFSSDAVSWVRQAPGEGLEWVSSISGSGVITYYADSVKGRFTISRDNSKNTLYLQMNSLRAEDTAVYYCAKDVFFFNYWGLGTLVTVSS

SEQ ID NO: 283 - protein, 116 aa, synthetic construct
DVQLVESGGGLVQPGGSLRLSCAASGFTFSDFAMSWVRQAPGEGLEWVSSISHSGVITFYADSVKGRFTISRDNSKNTLYLQMNSLRVEDTAIYYCSEGLSYHDYWGLGTLVTVSS

SEQ ID NO: 284 - protein, 116 aa, synthetic construct
DVQLVESGGGVVRPGESLRLSCAASGFTFSSDAMSWVRQAPGEGLEWVSSISGSGVITYYADSVKGRFTISRDNSKNTLYLQMNSLRAEDTAVYYCAKDVFFFNYWGLGTLVTVSS

SEQ ID NO: 285 - protein, 117 aa, synthetic construct
DVQLVESGGGVVRPGESLRLSCVASGFTFSSDVMSWVRQAPGEGLEWVSASSGSGVITYYADSVKGRFTISRDNSKNTLYLQMNSLRAEDTAVYYCAKAGNTFLDYWGLGTLVTVSS

SEQ ID NO: 286 - protein, 117 aa, synthetic construct
DVQLVESGGGVVRPGESLRLSCAASGFSFDIYDMSWVRQAPGKGLEWVSLISSSGVITYYADSVKGRFTISRDNSKSTLYLQMNSLRAEDTAVYYCARAGNTFFHYWGLGTLVTVSS

SEQ ID NO: 287 - protein, 116 aa, synthetic construct
DVQLVESGGGLVQPGGSLRLSCAASGFTFSDFAMSWVRQAPGEGLEWVSTISGSGVITFYADSVKGRFTISRDNSKNALYLQMNSLRVEDTAVYFCSEGMSYHDYWGLGTLVTVSS

SEQ ID NO: 288 - protein, 116 aa, synthetic construct
DVQLVESGGGVVRPGESLRLSCVASGFTFSTDTMSWVRQAPGEGLEWVSASSGSGVITYYADSVKGRFTISRDNSNNTLYLQMNSLRAEDTAIYYCATGFPFFDYWGLGTLVTVSS

SEQ ID NO: 289 - protein, 116 aa, synthetic construct
DVQLVESGGGLVQPGGSLRLSCAASGFTFSDFAMSWVRQAPGEGLEWVSTISGSGVITFYADSVKGRFTISRDNSKNTLYLQMNSLRVEDTAVYFCSEGMSYHDYWGLGTLVTVSS

SEQ ID NO: 290 - protein, 120 aa, synthetic construct
QVTLKESGPGILQPSQTLSLTCSFSGFSLSTFGMGVGWIRQPSGKGLEWLAHIWWDDDKYYNPALKSRLTISKDTSKNQVFLKIANVDTADTATYYCARIITTAWYFDVWGTGTTVTVSS

SEQ ID NO: 291 - protein, 117 aa, synthetic construct
QIQLVQSGPELKKPGETVKISCKASGYTFTTYGMSWVKQAPGKGLKWMGWINTYSGVPTYADDFKGRFAFSLETSASTAYLQINNLKNEDTATYFCARPRRQVFDYWGQGTTLTVSS

SEQ ID NO: 292 - protein, 121 aa, synthetic construct
QVKLQQSGAELVRPGASVKLSCKASGYTFTDYYINWIKQRPGQGLEWIARIYPVSGSTYYNDKFKGKATLTAEKSSSTAYMQLSSLTSEDSAVYFCVHIYYGNHPYYFDFWGQGTTLTVSS

SEQ ID NO: 293 - protein, 116 aa, synthetic construct
QVQLQQPGAELVKPGTSVKMSCKASGYTFITYWITWVKQRPGHGLEWIGDIYPGSGSTNYNAKFRSKATLTVDTSSSTAYMQFISLTSEDSAVYYCALKEIVPDYWGQGTTLTVSS

SEQ ID NO: 294 - protein, 120 aa, synthetic construct
QVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCARSRGNYVWYFDVWGTGTTVTVSS

SEQ ID NO: 295 - protein, 119 aa, synthetic construct
QVTLKESGPGILQPSQTLSLTCSFSGFSLSTFGMGVGWIRQPSGKGLEWLAHIWWDDDKYYNPALKSRLTISKDTSKNQVFLKIANVDTADTATYYCARMNPRNYFDYWGQGTTLTVSS

SEQ ID NO: 296 - protein, 116 aa, synthetic construct
QVQLQQPGAELVKPGASVKVSCKASGYTFTSYWMHWVKQRPGQGLEWIGRIHPSDSDTNYNQKFKGKATLTVDKSSSTAYMQLSSLTSEDSAVYYCAIRDWDLDYWGQGTTLTVSS

SEQ ID NO: 297 - protein, 117 aa, synthetic construct
EVMLVESGGGLVKPGGSLKLSCAASGFTFSSYLMSWVRQTPEKRLEWVASISGGGRDTYYPDSVKGRFTISRDNAKNTLYLQMSSLRSEDTALYYCARHGVGAMNYWGQGTSVTVSS

SEQ ID NO: 298 - protein, 116 aa, synthetic construct; identical to SEQ ID NO: 293

SEQ ID NO: 299 - protein, 120 aa, synthetic construct
QVTLKESGPGILQPSQTLSLTCSFSGFSLSTFGMGVGWIRQPSGKGLEWLAHIWWDDDKYYNPALKSRLTISKDTSKNQVFLKIANVDTADTATYYCARIPTRYWYFDVWGTGTTVTVSS

SEQ ID NO: 300 - protein, 121 aa, synthetic construct
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARSRDYDGLYAMDYWGQGTSVTVSS

SEQ ID NO: 301 - protein, 119 aa, synthetic construct
QVTLKESGPGILQPSQTLSLTCSFSGFSLSTFGMGVGWIRQPSGKGLEWLAHIWWDDDKYYNPALKSRLTISKDTSKNQVFLKIANVDTADTATYYCARKWNYWYFDVWGTGTTVTVSS

SEQ ID NO: 302 - protein, 118 aa, synthetic construct
QVQLQQPGAELVKPGASVKMSCKASGDTFTSYWITWVKQRPGQGLEWIGDIYPGSGSTNYNEKFKSKATLTVDTSSSTAYMQLSSLTSEDSAVYYCARKWNYWYFDVWGTGTTVTVSS

SEQ ID NO: 303 - protein, 121 aa, synthetic construct
QVQLQQPGAELVKPGASVKMSCKASGDTFTSYWITWVKQRPGQGLEWIGDIYPGSGSTNYNEKFKSKATLTVDTSSSTAYMQLSSLTSEDSAVYYCARRYYHGSSWYFDVWGTGTTVTVSS

SEQ ID NO: 304 - protein, 118 aa, synthetic construct
QIQFVQSGPELKKPGETVKISCKASVYTFTEYPIHWVKQAPGKGFKWMGCINTYSGEPTYADDFKRRFAFSLETSASTAYLQINNLKNEDTATYFCARCYGYDVFDYWGQGTTLTVSS

SEQ ID NO: 305 - protein, 120 aa, synthetic construct
QVQLQQSGAELVRPGASVTLSCKASGYTFTDYEMHWVKQTPVHGLEWIGTIDPETGGTAYNQKFKGKAILTADKSSSTAYMVLRSLTSEDSAVYYCTREGYGSPYYFDYWGQGTTLTVSS

SEQ ID NO: 306 - protein, 120 aa, synthetic construct
QVTLKESGPGILQPSQTLSLTCSFSGFSLSTFGMGVGWIRQPSGKGLEWLAHIWWDDDKYYNPALKSRLTISKDTSKNQVFLKIANVDTADTATYYCARIAEGRWYFDVWGTGTTVTVSS

SEQ ID NO: 307 - protein, 114 aa, synthetic construct
QVQLQQPGAELVMPGASVKLSCKASGYTFTSYWMHWVKQRPGQGLEWIGEIDPSDSYTNYNQKFKGKSTLTVDKSSSTAYMQLSSLTSEDSAVYYCARSPFDYWGQGTTLTVSS

SEQ ID NO: 308 - protein, 120 aa, synthetic construct
EVQLQQSGPELVKPGASVKIPCKASGYTFTDYNMDWVKQSHGKSLEWIGDINPNNGGTIYNQKFKGKATLTVDKSSSTAYMELRSLTSEDTGVYYCVTSIYYDSAWFGYWGQGTLVTVSA

SEQ ID NO: 309 - protein, 114 aa, synthetic construct
QIQLVQSGPELKKPGETVKISCKASGYTFTTYGMSWVKQAPGKGLKWMGWINTYSGVPAYAADFKGRFAFSLETSASTAYLQINTLKNEDTATYFCARGGNPYWGQGTLVTVSA

SEQ ID NO: 310 - protein, 120 aa, synthetic construct
QVTLKESGPGILQPSQTLSLTCSFSGFSLSTFGMGVGWIRQPSGKGLEWLAHIWWDDDKYYNPALKSRLTISKDTSKNQVFLKIANVDTADTATYYCARIRGTWWYFDVWGTGTTVTVSS

SEQ ID NO: 311 - protein, 121 aa, synthetic construct
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCARRGGSSSYWYFDVWGTGTTVTVSS

SEQ ID NO: 312 - protein, 121 aa, synthetic construct
QVTLKESGPGILQPSQTLSLTCSFSGFSLSTFGMGVGWIRQPSGKGLEWLAHIWWDDDKYYNPALKSRLTISKDTSKNQVFLKIANVDTADTATYYCARKVGRPLWYFDVWGAGTTVTVSS

SEQ ID NO: 313 - protein, 118 aa, synthetic construct
QVQLKESGPGLVAPSQSLSITCTVSGFSLTSYAISWVRQPPGKGLEWLGVIWTGGGTNYNSALKSRLSISKDNSKSQVFLKMNSLQTDDTARYYCASSKGSYYAMDYWGQGTSVTVSS

SEQ ID NO: 314 - protein, 114 aa, synthetic construct
QIQLVQSGPELKKPGATVKISCKASGFTFTTYGMSWVKQAPGKGFKWMGWLNTYSGVPTYADDFKGRFAFSLETSASTAYLQINNLKNEDTATYFCARGGYPYWGRGTTLTVSS

SEQ ID NO: 315 - protein, 119 aa, synthetic construct
EVQLQQSGAELVKPGASVKLSCTASGFNIKDYYMHWVKQRTEQGLEWIGRIDPEDGETKYAPKFQGKATITADTSSNTAYLQLSSLTSEDTAVYYCGRDGYYDVYFDYWGQGTTLTVSS

SEQ ID NO: 316 - protein, 112 aa, synthetic construct
QVQLQQSGAELVKPGASVKISCKTSGYIFSNYWMNWVKQRPGKGLEWIGQIYPGDGDTNYNGDFKGKATLTADKSSSTAYIYLNSLTSEDSAVYFCARGPYWGLGTLVTVSA

SEQ ID NO: 317 - protein, 120 aa, synthetic construct
QVTLKESGPGILQPSQTLSLTCSFSGFSLSTFGMGVGWIRQPSGKGLEWLAHIWWDDDKYYNPALKSRLTISKDTSKNQVFLKIANVDTADTATYYCARIQSFYWYFDVWGTGTTVTVSS

SEQ ID NO: 318 - protein, 119 aa, synthetic construct
EVQLQQSGAELVKPGASVKLSCTASGFNIKDYYMHWVKQRTEQGLEWIGRIDPEDGETKYAPKFQGKTTITADTSSNTAYLQLSSLTSEDTAVYYCGRDGYYDVYFDYWGQGTTLTVSS

SEQ ID NO: 319 - protein, 118 aa, synthetic construct
QIQLVQSGPELKKPGETVKISCKASGYTFTTYGMSWVKQAPGKGLKWMGWINTYSGLPTYADDFKGRFAFSLETSASTAYLQINNLKNEDTATYFCARGGYDYGAAYWGQGTLVTVSA

SEQ ID NO: 320 - protein, 120 aa, synthetic construct
QVTLKESGPGILQPSQTLSLTCSFSGFSLSTFGMGVGWIRQPSGKGLEWLTHIWWDDDKYYNPALKSRLTISKDTSKNQVFLKIANVDTADTATYYCARVRMSHWYFDVWATGTTVTVSS

SEQ ID NO: 321 - protein, 121 aa, synthetic construct
QVQLQQSGAELMKPGASVKLSCKATGYTFTGYWIEWVKQRPGHGLEWIGEILPGSGSTNYNEKFKGKATFTADTSSNTAYMQLSSLTTEDSAIHYCARKEDGYYLYYFDYWGQGTTLTVSS

SEQ ID NO: 322 - protein, 115 aa, synthetic construct
QVQLQQSGAVLMKPGASVKLSCKATGYTITGSWIAWLKQRPGHGLEWIGEILPGSGRTNLNEDFKGKATFTADTSSNTVFIQLSSLTTEDSAIYYCYNGSAFDYWGQGTTLTVSS

SEQ ID NO: 323 - protein, 108 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTKVEIK

SEQ ID NO: 324 - protein, 110 aa, synthetic construct
QSVLTQPPSASGTPGQRVTISCSGSSSNIGSKYVSWYQQLPGTAPKLLIYKDDQRPSGVPDRFSGSKSGTSASLAISGLQSEDEADYYCAAWDGSLNGWVFGGGTKLTVL

SEQ ID NO: 325 - protein, 110 aa, synthetic construct
QSVLTQPPSVSGTPGQRVTISCSGSNSNIGGYYVSWYQQVPGTAPKLLIYKNFQRPSGVPDRFSGSKSGTSASLAISGLQSEDEADYYCASWDGSLNGWVFGGGTKLTVL

SEQ ID NO: 326 - protein, 110 aa, synthetic construct
QSVLTQPPSASGTPGQRVTISCSGSTSNIGTRYVYWYQQLPGTAPKLLIYKNLQRPSGVPDRFSGSKSGTSASLAISGLQSEDEADYYCAAWDERLNGWVFGGGTKLTVL

SEQ ID NO: 327 - protein, 110 aa, synthetic construct
QSVLTQPPSASGTPGQRVTISCSGSSSNIGRRYVYWYQQLPGTAPKLLIYRNFERPSGVPDRFSGSKSGTSASLAISGLQSEDEADYYCASWDESLNGWVFGGGTKLTVL

SEQ ID NO: 328 - protein, 110 aa, synthetic construct
QSVLTQPPSASGTPGQRVTISCSGSTSNIGRRYVYWYQQLPGTAPKLLIYRNFERPSGVPDRFSGSKSGTSASLAISGLQSEDEADYYCASWDESLNGWVFGGGTKLTVL

SEQ ID NO: 329 - protein, 110 aa, synthetic construct
QSVLTQPPSASGTPGQRVTISCSGSSSNIRNNYVSWYQQLPGTAPKLLIYKYNQRPSGVPDRFSGSKSGTSASLAISGLQSEDEADYYCGTWDDSLNGFVFGGGTKLTVL

SEQ ID NO: 330 - protein, 110 aa, synthetic construct
QSVLTQPPSASGTPGQRVTISCSGSSSNIGNNYVSWYQQLPGTAPKLLIYKDDQRPSGVPDRFSGSKSGTSASLAISGLQSEDEADYYCAARVDTLNVWVFGGGTKLTVL

SEQ ID NO: 331 - protein, 110 aa, synthetic construct
QSVLTQPPSASGTPGQRVTISCSGSSSNIRNNYVSWYQQLPGTAPKLLIYRNSQRPSGVPDRFSGSKSGTSASLAISGLQSEDEADYYCAAWDDSLNGWVFGGGTKLTVL

SEQ ID NO: 332 - protein, 110 aa, synthetic construct
QSVLTQPPSASGTPGQRVTISCSGSSPNIRNNYVSWYQQLPGTAPKLLIYKYNQRPSGVPDRFSGSKSGTSASLAISGLQSEDEADYYCGTWDDSLNGFVFGGGTKLTVL

SEQ ID NO: 333 - protein, 110 aa, synthetic construct
QSVLTQPPSASGTPGQRVTISCSGSTSNIGTNRVYWYQRLPGTAPKLLIYRDQERPSGVPDRFSGSKSGTSASLAISGLQSEDEADYYCASWDDSLNAWVFGGGTKLTVL

SEQ ID NO: 334 - protein, 109 aa, synthetic construct
QSVLTQPPSASGAPGQRVTISCSGSYSNIGNSYVSWYQQPPGTAPKLLIYLNSQRPSGVPDRFSGSKSGTSASLAISGLQSEDEADYYCAAWDDLNVWVFGGGTKLTVL

SEQ ID NO: 335 - protein, 109 aa, synthetic construct
QSVLTQPPSASGTPGQRVTISCSGSYSNIGNSYVSWYQQLPGTAPKLLIYLNSQRPSGVPDRFSGSKSGTSASLAISGLRSEDEADYYCAAWDDLNVWVFGGGTKLTVL

SEQ ID NO: 336 - protein, 110 aa, synthetic construct
QSVLTQPPSASGTPGQRVTISCSGSRSNIGTKYVSWYQQLPGTAPKLLIYKDDQRPSGVPDRFSGSQSGTSASLAISGLQSEDEADYYCGTWDDSLNVWVFGGGTKLTVL

SEQ ID NO: 337 - protein, 110 aa, synthetic construct
QSVLTQPPSASGTPGQRVTISCSGSRSNIGTNRVYWYQQLPGTAPKLLIYKDDQRPSGVPDRFSGSKSGTSASLAISGLQSEDEADYYCAAWDDSLNGWVFGGGTKLTVL

SEQ ID NO: 338 - protein, 109 aa, synthetic construct
QSVLTQPPSASGTPGQRVTISCSGSYSNIGNSYVSWYQQLPGTAPKLLIYLNSQRPSGVPDRFSGSKSGTSASLAISGLQSEDEADYYCAAWDDPNVWVFGGGTKLTVL

SEQ ID NO: 339 - protein, 110 aa, synthetic construct
QSVLTQPPSASGTPGQRVTISCSGSSSNIDINYIDWYQQLPGTAPKVLIYNTDQRPSGVPDRFSGSKSGTSASLAISGLQSEDEADYYCAGWDSSLRVWVFGGGTKLTVL

SEQ ID NO: 340 - protein, 111 aa, synthetic construct
QSVLTQPPSASGTPGQRVTISCSGSTSNIGRNNYVSWYQQLPGTAPKLLIYRDYQRPSGVPDRFSGSKSGTSASLAISGLQSEDEADYYCVAWDDRVNGWVFGGGTKLTVL

SEQ ID NO: 341 - protein, 110 aa, synthetic construct
QSVLTQPPSASGTPGQRVTISCSGSTSNIRNNYVSWYQQLPGTAPKLLIYGKNQRPSGVPDRFSGSKSGTSASLAISGLQSEDEADYYCATWDDSLNGWVFGGGTKLTVL

SEQ ID NO: 342 - protein, 110 aa, synthetic construct
QSVLTQPPSASGTPGQRVTISCSGSTSNIRNNYVSWYQQLPGTAPKLLIYGKNQRPSGVPDRFSGSKSGTSASLAISGLQSEDEADYYCSTWDDSLNGWVFGGGTKLTVL

SEQ ID NO: 343 - protein, 109 aa, synthetic construct
QSVLTQPPSASGTPGQRVTISCSGSYSNIGNSYVSWYQQLPGTAPKLLIYLNSQRPSGVPDRFSGSKSGTSASLAISGLQSEDEADYYCAAWDDLNVWVFGGGTKLTVL

SEQ ID NO: 344 - protein, 109 aa, synthetic construct
QSVLTQPPSASGAPGQRVTISCSGSYSNIGNSYVSWYQQLPGTAPKLLIYLNSQRPSGVPDRFSGSKSGTSASLAISGLQSEDEADYYCAAWDDLNVWVFGGGTKLTVL

SEQ ID NO: 345 - protein, 111 aa, synthetic construct
QSVLTQPPSASGTPGQRVTISCSGSTSNIGRNNYVSWYQQLPGTAPKLLIYRDYQRPSGAPDRFSGSKSGTSASLAISGLQSEDEADYYCVAWDDRVNGWVFGGGTKLTVL

SEQ ID NO: 346 - protein, 110 aa, synthetic construct
QSVLTQPPSASGTPGQRVTISCSGSRSNIGTNRVYWYQQLPGTAPKLLIYKDDQRPSGVPDRFSGSKSGTSASLAISGLQSEDEADYYCASWDDSLNGWVFGGGTKLTVL

SEQ ID NO: 347 - protein, 110 aa, synthetic construct
QSVLTQPPSASGTPGQRVTISCSGSSFNIGTRYVYWYQQLPGTAPKLLIYRNSQRPSGVPDRFSGSKSGTSASLAISGLQAEDEADYYCAAWDDSLNGWVFGGGTKLTVL

SEQ ID NO: 348 - protein, 110 aa, synthetic construct
QSVLTQPPSASGTPGQRVTISCSGSTSNIGTRYVYWYQQLPGTAPKLLIYRNFERPSGVPDRFSGSKSGTSASLAISGLQSEDEADYYCAAWDDRVNGWLFGGGTKLTVL

SEQ ID NO: 349 - protein, 110 aa, synthetic construct
QSVLTQPPSASGTPGQRVTISCSGSSSNIGTNTVDWYQQLPGTAPKLLIYDNFERPSGVPDRFSGSKSGISASLAISGLQSEDEADYYCGTWDDSLNAWVFGGGTKLTVL

SEQ ID NO: 350 - protein, 110 aa, synthetic construct
QSVLTQPPSASGTPGQRVTISCSGSNSNIGRRYVYWYQQLPGTAPKLLIYKNFERPSGVPDRFSGPKSGTSASLAISGLRSEDEADYYCASWDESLNGWVFGGGTKLTVL

SEQ ID NO: 351 - protein, 110 aa, synthetic construct
QSVLTQPPSASGTPGQRVTISCSGSTSNIGRRYVYWYQQLPGTAPKLLIYRNFERPSGVPDRFSGSKSGTSASLAISGLQSEDEADYYCGTWDDSLNSWVFGGGTKLTVL

SEQ ID NO: 352 - protein, 110 aa, synthetic construct
QSVLTQPPSASGTPGQRVTISCSGSTFNIGTNYVSWYQQLPGTAPKLLIYKNHQRPSGVPDRFSASKSGTSASLAISGLQSEDEADYYCAAWDDSLNVRVFGGGTKLTVL

SEQ ID NO: 353 - protein, 110 aa, synthetic construct
QSVLTQPPSASGTPGQRVTISCSGSSSNIGTRYVYWYQQLPGTAPKLLIYRNFERPSGVPDRFSGSKSGTSASLAISGLQSEDEADYYCASWDDSLNGWVFGGGTKLTVL

SEQ ID NO: 354 - protein, 110 aa, synthetic construct
QSVLTQPPSASGTPGQRVTISCSGSSSNIGRRYVYWYQQLPGTAPKLLIYRNFERPSGVPDRFSGSKSGTSASLAISGLQSEDEAYYYCASWDESLNGWVFGGGTKLTVL

SEQ ID NO: 355 - protein, 110 aa, synthetic construct
QSVLTQPPSASGTPGQRVTISCSGTIFNIGTNYVSWYQQLPGTAPKLLIYLNSQRPSGVPDRFSASKSGTSASLAISGLQSEDEADYYCAAWDDSLNVRVFGGGTKLTVL

SEQ ID NO: 356 - protein, 110 aa, synthetic construct
QSVLTQPPSASGTPGQRVTISCSGSTSNIGRRYVYWYQQLPGTAPKLLIYRNFERPSGVPDRFSGSKSGTSASLAISGLQSEDEADYYCAAWDESLNGWVFGGGTKLTVL

SEQ ID NO: 357 - protein, 111 aa, synthetic construct
QSVLTQPPSVSGTPGQRVTISCSGTISNIGNDNRVHWYQQLPGTAPKLLIYGNHQRPSGVPDRFSGSKSGTSASLAISGLQSEDEADYYCGTWDDSLNTWVFGGGTKLTVL

SEQ ID NO: 358 - protein, 110 aa, synthetic construct
QSVLTQPPSASGTPGQRVTISCSGTTFNIGRRYVYWYQQLPGTAPKLLIYRNFERPSGVPDRFSGSKSGTSASLAISGLQSEDEADYYCASWDESLNGWVFGGGTKLTVL

SEQ ID NO: 359 - protein, 110 aa, synthetic construct
QSVLTQPPSASGTPGQRVTISCSGSYSNIGNNYVYWYQQLPGTAPKLLIYKDDQRPSGVPDRFSGSKSGTSASLAISGLQSEDEADYYCGAFDDSLNVWVFGGGTKLTVL

SEQ ID NO: 360 - protein, 110 aa, synthetic construct
QSVLTQPPSASGTPGQRVTISCSGSTFNIQSNYVYWYQQLPGTAPKLLIYQSHVRPSGVPDRFSGSKSGTSASLAISGLQSEDEADYYCSSWDASLNGWVFGGGTKLTVL

SEQ ID NO: 361 - protein, 110 aa, synthetic construct
QSVLTQPPSASGTPGRRVTISCSGSTSNIGTRYVYWYQQLPGTAPKLLIYRNFERPSGVPDRFSGSKSGTSASLAISGLQSEDEADYYCAAWDDRVNGWLFGGGTKLTVL

SEQ ID NO: 362 - protein, 110 aa, synthetic construct
QSVLTQPPSASGTPGQRVTISCSGGSSNIGTNTVDWYQQLPGTAPKLLIYDNFERPSGVPDRFSGSKSGTSASLAISGLQSEDEADYYCGTWDDSLNAWVFGGGTKLTVL

SEQ ID NO: 363 - protein, 110 aa, synthetic construct
QSVLTQPPSASGTPGQRVTISCSGSNSNIGRRYVYWYQQLPGTAPKLLIYRNFERPSGVPDRFSGSKSGTSASLAISGLQSEDEADYYCASWDESLNGWVFGGGTKLTVL

SEQ ID NO: 364 - protein, 110 aa, synthetic construct
QSVLTQPPSASGTPGQRVTMSCSGSSSNIGNRYVYWYQQFPGTAPKLLIHHNSQRPSGVPDRFSGSKSGTSASLAISGLQSEDEADYYCASWDDSLNGWVFGGGTKLTVL

SEQ ID NO: 365 - protein, 110 aa, synthetic construct
QSVLTQPPSASGTPGQRVTISCSGSTSNIGRRYVYWYQQLPGTAPKLLIYRNSQRPSGVPDRFSGSKSGTSASLAISGLQSEDEADYYCASWDESLNGWVFGGGTKLTVL

SEQ ID NO: 366 - protein, 110 aa, synthetic construct
QSVLTQPPSASGTPGQRVTISCSGSSSNIGTNTVDWYQQLPGTAPKLLIYDNFERPSGVPDRFSGSKSGTSASLAISGLQSEDEADYYCGTWDDSLNAWVFGGGTKLTVL

SEQ ID NO: 367 - protein, 110 aa, synthetic construct
QSVLTQPPSASGTPGQRVTISCSGSNSNIGDHYVDWYQQLPGTAPKLLIYNNVQRPSGVPDRFSGSKSGTSASLAISGLQSEDEADYYCATWDDNLNGWVFGGGTKLTVL

SEQ ID NO: 368 - protein, 110 aa, synthetic construct
QSVLTQPPSASGTPGQRVTISCSGSSYNIDYNYIDWYQQLPGTAPKLLIYNSDQRPSGVPDRFSGSKSGTSASLAISGLQSEDEADYYCASWDAGLNVWMFGGGTKLTVL

SEQ ID NO: 369 - protein, 110 aa, synthetic construct
QSVLTQPPSASGTPGQRVTISCSGSTSNIGRRYVYWYQQLPGTAPKLLIYRNFERPSGVPDRFSGSKSGTSASLAISGLQSEDEADYYCASWDERLNGWVFGGGTKLTVL

SEQ ID NO: 370 - protein, 110 aa, synthetic construct
QSVLTQPPSASGTPGQRVTISCSGSTSNIGRRYVYWYQQLPGTAPKLLIYRNFERPSGVPDRFSGSKSGTSASLAISGLQAEDEADYYCASWDESLNGWVFGGGTKLTVL

SEQ ID NO: 371 - protein, 110 aa, synthetic construct
QSVLTQPPSASGTPGQRVTISCSGSNSSIGRRYVYWYQQLPGTAPKLLIYRNFERPSGVPDRFSGSKSGTSASLAISGLQSEDEADYYCASWDESLNGWVFGGGTKLTVL

SEQ ID NO: 372 - protein, 110 aa, synthetic construct
QSVLTQPPSASGTPGQRVTISCSGSTSNIGRRYVYWYQQVPGTAPKLLIYRNFERPSGVPDRFSGSKSGTSASLAISGLQSEDEADYYCASWDESLNGWVFSGGTKLTVL

SEQ ID NO: 373 - protein, 111 aa, synthetic construct
QSVLTQQPAPVSGTPGQRVTISCSGSSSNIGDHYVDWYQQLPGTAPKLLIYDNSQRPSGVPDRFSGSKSGTSASLAISGLQSEDEADYYCGTWDDDLNVWVFGGGTKLTVL

SEQ ID NO: 374 - protein, 110 aa, synthetic construct
QSVLTQPPSASGTPGQRVTISCSGSTSNIGRRYVYWYQQFPGTAPKLLIYRNSQRPSGVPDRFSGSKSGTSASLAISGLQSEDEADYYCASWDESLNGWVFGGGTKLTVL

SEQ ID NO: 375 - protein, 110 aa, synthetic construct
QSVLTQPPSASGTPGQRVTMSCSGSSSNIGNRYVYWYRQFPGTAPKLLIHHNSQRPSGVPDRFSGSKSGTSASLAISGLQSEDEADYYCASWDDSLNGWVFGGGTKLTVL

SEQ ID NO: 376 - protein, 110 aa, synthetic construct
QSVLTQPPSASGTPGQRVTISCSGSSSNIGTRYVYWYQQVPGTAPKLLIYRNFERPSGVPDRFSGSKSGTSASLAISGLQSEDEADYYCAAWDDSLNGWVFGGGTKLTVL

SEQ ID NO: 377 - protein, 110 aa, synthetic construct
QSVLTQPPSASGTPGQRVTISCSGGTSNIGTRYVYWYQQLPGTAPKLLIYRNFERPSGVPDRFSGSKSGTSASLAISGLQSEDEADYYCAAWDDRVNGWLFGGGTKLTVL

SEQ ID NO: 378 - protein, 111 aa, synthetic construct
QSVLTQQPASVSGTPGQRVTISCSGSSSNIGDHYVDWYQQLPGTAPKLLIYDNSQRPSGVPDRFSGSKSGTSASLAISGLQSEDEADYYCGTWDDDLNVWVFGGGTKLTVL

SEQ ID NO: 379 - protein, 110 aa, synthetic construct
QSVLTQPPSASGTPGQRVTISCSGSTSNIGRRYVYWYQQLPGTAPKLFIYRNFERPSGVPDRFSGSKSGTSASLAISGLQAEDEADYYCASWDESLNGWVFGGGTKLTVL

SEQ ID NO: 380 - protein, 110 aa, synthetic construct
QSVLTQPPSASGTPGQRVTISCSGSNSNIDYNYIDWYQQLPGTAPKLLIYNNDQRPSEVPDRFSGSKSGTSASLAISGLQSEDEADYYCGTWDDSLNAWVFGGGTKLTVL

SEQ ID NO: 381 - protein, 110 aa, synthetic construct
QSVLTQPPSASGTPGQRVTISCSGSTSNIGRRYVYWYQQLPGTAPKLLIYRNFERPSGVPDRFSGSKSGTSASLAISGLQSEDEADYYCAAWDDSLNAWVFGGGTKLTVL

SEQ ID NO: 382 - protein, 110 aa, synthetic construct
QSVLTQPPSASGTPGQRVTISCSGSTSNIRTRYVYWYQQLPGTAPKLLIYRNFERPSGVPDRFSGSKSGTSASLAISGLQSEDEADYYCAAWDDRVNGWLFGGGTKLTVL

SEQ ID NO: 383 - protein, 110 aa, synthetic construct
QSVLTQPPSASGTPGQRVTISCSGSTSNIGTRYVYWYQQLPGTAPKLLIYRNFERPSGVPDRFSGSKSGTSASLAISGLQSEDEANYYCAAWDDSLNGWVFGGGTKLTVL

SEQ ID NO: 384 - protein, 110 aa, synthetic construct
QSVLTQPPSASGTPGQRVTISCSGSNSNIDYNYIDWYQQLPGTAPKLLIYNNDQRPSEVPDRFSGSKSGTSASLAISGLQSEDEADYYCAAWDDNLNSWVFGGGTKLTVL

SEQ ID NO: 385 - protein, 110 aa, synthetic construct
QSVLTQPPSASGTPGQRVTISCSGSNSNIDYNYIDWYQQLPGTAPKLLIYNNDQRPSEVPDRFSGSKSGTSASLAISGLRSEDEADYYCATWDDNLNSWVFGGGTKLTVL

SEQ ID NO: 386 - protein, 110 aa, synthetic construct
QSVLTQPPSASGTPGQRVTISCSGSTSNIGRRYVYWYQQVPGTAPKLLIYRNFERPSGVPDRFSGSKSGTSASLAISGLQSEDEADYYCASWDESLNGWVFGGGTKLTVL

SEQ ID NO: 387 - protein, 110 aa, synthetic construct
QSVLTQPPSASGTPGQRVTISCSGSTFNIGNNYVSWYQQLPGTAPKVLIYKNLQRPSGVPDRFSGSKSGTSASLAISGLQSEDEADYYCASWDDSLNVRVFGGGTKLTVL

SEQ ID NO: 388 - protein, 109 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHFIFGSGTKVTVL

SEQ ID NO: 389 - protein, 107 aa, synthetic construct
DIQMTQSPASLSASVGETVTITCRASENIYSYLAWYQQKQGKSPQLLVYNAKTLAEGVPSRFSGSGSGTQFSLKINSLQPEDFGSYYCQHHYGTPFTFGSGTKLEIK

SEQ ID NO: 390 - protein, 110 aa, synthetic construct
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELYTFGGGTKLEIK

SEQ ID NO: 391 - protein, 109 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHLVFGGGTKLTVL

SEQ ID NO: 392 - protein, 111 aa, synthetic construct
DIVLTQSPASLAVSLGQRATISCRASQSVSTSSYSYMHWYQQKPGQPPKLLIKYASNLESGVPARFSGSGSGTDFTLNIHPVEEEDTATYYCQHSWEIPFTFGSGTKLEIK

SEQ ID NO: 393 - protein, 110 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHSWVFGGGTKLTVL

SEQ ID NO: 394 - protein, 106 aa, synthetic construct
ENVLTQSPAIMSASLGEKVTMSCRASSSVNYMYWYQQKSDASPKLWIYYTSNLAPGVPARFSGSGSGNSYSLTISSMEGEDAATYYCQQFTSSPSTFGGGTKLEIK

SEQ ID NO: 395 - protein, 109 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSNRWVFGGGTKLTVL

SEQ ID NO: 396 - protein, 107 aa, synthetic construct
DIQMTQSPASQSASLGESVTITCLASQTIGTWLAWYQQKPGKSPQVLIYAATSLADGVPSRFSGSGSGTKFSFKISSLQAEDFVSYYCQQLYSTPWTFGGGTKLEIK

SEQ ID NO: 397 - protein, 109 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSTHYVFGGGTKVTVL

SEQ ID NO: 398 - protein, 109 aa, synthetic construct
QAVVTQESALTTSPGGTVILTCRSSTGAVTTSNYANWVQEKPDHLFTGLIGGTSNRAPGVPVRFSGSLIGDKAALTITGAQTEDDAMYFCALWYSTHYVFGGGTKVTVL

SEQ ID NO: 399 - protein, 107 aa, synthetic construct
DIQMTQSPASLSASVGETVTITCGASENIYGGLNWYQRKQGKSPQLLIYGATNLADGMSSRFSGSGSGRQYSLKIRSLHPDDVATYYCQNVLSIPYTFGGGTKLEIK

SEQ ID NO: 400 - protein, 111 aa, synthetic construct
DIVLTQSPASLAVSLGQRATISCRASESVDNYGISFMKWFQQKPGQSPKLLIYAASNQGSGVPARFSGSGSGTDFSLNIHPMEEDDTAMYFCQQSKEVPRTFGGGTKLEVK

SEQ ID NO: 401 - protein, 107 aa, synthetic construct
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPYTFGGGTKLEIK

SEQ ID NO: 402 - protein, 107 aa, synthetic construct
DIQMTQTTSSLSVSLGDRVTISCSASQVITNYLNWYQQKPDGTIKLLIYYTSSLHSGVPSRFSGSGSGTDYSLTISNLEPEDIATYFCQQYDKLPWTFGGGTKLEIK

SEQ ID NO: 403 - protein, 109 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSNQFIFGSGTKVTVL

SEQ ID NO: 404 - protein, 108 aa, synthetic construct
ENVLTQSQAIMSASPGEKVTMTCRASSSVSSSYLHWYQQKSGASPKLWIYSTSNLASGVPARFSGSGSGTSYSLTISSVEAEDAATYYCQQYSGYPLTFGAGTKLELK

SEQ ID NO: 405 - protein, 111 aa, synthetic construct
DIVLTQSPASLAVSLGQRATISCRASQSVSTSSYSYMHWYQQKPGQPPKLLIKYASNLESGVPARFSGSGSGTDFTLNIHPVEEEDTATYYCQHSWEIPPTFGSGTKLEIK

SEQ ID NO: 406 - protein, 107 aa, synthetic construct
DIQMTQSPASQSASLGESVTITCLASQTIGTWLAWYQQKPGKSPQLLIYAATSLADGVPSRFSGSGSGTKFSFKISSLQAEDFVSYYCQQFYSTPWTFGGGTKLEIK

SEQ ID NO: 407 - protein, 107 aa, synthetic construct
DIQMTQSPASQSASLGESVTITCLASQTIGTWLAWYQQKPGKSPQLLIYAATSLADGVPSRFSGSGSGTKFSFKISSLQAEDFESYYCQQFYSTPWTFGGGTKLEIK

SEQ ID NO: 408 - protein, 111 aa, synthetic construct
DIVLTQSPASLAVSLGQRATISCRASQSVSTSSYSYMHWYQQKPGQPPKLLIKYASNLESGVPARFSGSGSGTDFTLNIHPVEEEDTATYYCQHSWEIPYTFGGGTKLEIK

SEQ ID NO: 409 - protein, 109 aa, synthetic construct
QAVVTQESALTTSPGGTVILTCRSSTGAVTTSYYANWVQEKPDHLFTGLIGGTSNRAPGVPVRFSGSLIGDKAALTITGAQTEDDAMYFCALWYSTHYVFGGGTKVTVL

SEQ ID NO: 410 - protein, 106 aa, synthetic construct
QIVLTQSPTIMSASPGEKVTMTCSASLSLSSMFWYQQKPGSSPRLLIYDTSKLASGVPVRFSGSGSGTSYSLTISRMEAEDGATYYCQQWSSFPFTFGSGTKLEIK

SEQ ID NO: 411 - protein, 107 aa, synthetic construct
DIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEIK

SEQ ID NO: 412 - protein, 109 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHYIFGSGTKVTVL

SEQ ID NO: 413 - protein, 112 aa, synthetic construct
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPHTFGAGTKLELK

SEQ ID NO: 414 - protein, 107 aa, synthetic construct
DIQMTQTTSSLSASLGDRVTISCRASQDISNYLNWYQQKPDGTVKLLIYYTSRLHSGVPSRFSGSGSGTDYSLTIRNLDQEDIATYFCQQDKTFPWTFGGGTKLEIK

SEQ ID NO: 415 - protein, 107 aa, synthetic construct [sequence omitted]

SEQ ID NO: 416 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 417 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGFSVFLFPPKPKDTLMISRTPEVTCVVVDVKHEDPEVKFNWYVDGVEVHNAKTKPREEQYNSTYRVVSVLTVLHQDWLNGKEYKCKVSNKAMPEPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 418 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGFSVFLFPPKPKDTLMISRTPEVTCVVVDVKHEDPEVKFNWYVDGVEVHNAKTKPREEQYNSTYRVVSVLTVLHQDWLNGKEYKCKVSNKDMPEPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 419 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGHFSVFLFPPKPKDTLMISRTPEVTCVVVDVKHEDPEVKFNWYVDGVEVHNAKTKPREEQYNSTYRVVSVLTVLHQDWLNGKEYKCKVSNKDMPEPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 420 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGMFSVFLFPPKPKDTLMISRTPEVTCVVVDVKHEDPEVKFNWYVDGVEVHNAKTKPREEQYNSTYRVVSVLTVLHQDWLNGKEYKCKVSNKDMPEPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 421 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 422 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELGQGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 423 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEPGQGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 424 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELGQGPSVFLFPPKPKDTLMISRTPEVTCVVVDVSHEDPEVKFNWYVDGVEVHNAKTKPREEQYNSTYRVVSVLTVLHQDWLNGKEYKCKVSNKAWRAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 425 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELGQRPSVFLFPPKPKDTLMISRTPEVTCVVVDVSHEDPEVKFNWYVDGVEVHNAKTKPREEQYNSTYRVVSVLTVLHQDWLNGKEYKCKVSNKAWRAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 426 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELGQGPSVFLFPPKPKDTLMISRTPEVTCVVVDVSHEDPEVKFNWYVDGVEVHNAKTKPREEQYNSTYRVVSVLTVLHQDWLNGKEYKCKVSNKALDAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 427 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPLKLGAPSVFLFPPKPKDTLMISRTPEVTCVVVDVSPENPEVKFNWYVDGVEVHNAKTKPREEQYNSTYRVVSVLTVLHQDWLNGKEYKCKVSTSTIPGQIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 428 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPMGGGAPSVFLFPPKPKDTLMISRTPEVTCVVVDVSPEDPEVEFNWYVDGVEVHNAKTKPREEQYNSTYRVVSVLTVLHQDWLNGKEYKCKVSTPSQPADIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 429 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPITPGAPSVFLFPPKPKDTLMISRTPEVTCVVVDVSPEAPEVEFNWYVDGVEVHNAKTKPREEQYNSTYRVVSVLTVLHQDWLNGKEYKCKVSTAGLGSNIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 430 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPRTPALPSVFLFPPKPKDTLMISRTPEVTCVVVDVSPEDPEVEFNWYVDGVEVHNAKTKPREEQYNSTYRVVSVLTVLHQDWLNGKEYKCKVSNGEIREHIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 431 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGLPSVFLFPPKPKDTLMISRTPEVTCVVVDVSHEDPEVKFNWYVDGVEVHNAKTKPREEQYNQTYRVVSVLTVLHQDWLNGKEYKCKVSNKDLPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 432 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGLPSVFLFPPKPKDTLMISRTPEVTCVVVDVSHEDPEVKFNWYVDGVEVHNAKTKPREEQYNQRYRVVSVLTVLHQDWLNGKEYKCKVSNKDLPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 433 - skipped

SEQ ID NO: 434 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLPLPSVFLFPPKPKDTLMISRTPEVTCVVVDVSHEDPEVKFNWYVDGVEVHNAKTKPREEQYNQTYRVVSVLTVLHQDWLNGKEYKCKVSNKDLPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 435 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGLPSVFLFPPKPKDTLMISRTPEVTCVVVDVSHEDPEVKFNWYVDGVEVHNAKTKPREEQYNQTYRVVSVLTVLHQDWLNGKEYKCKVSDKDLPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 436 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEMLPLPSVFLFPPKPKDTLMISRTPEVTCVVVDVSHEDPEVKFNWYVDGVEVHNAKTKPREEQYNQTYRVVSVLTVLHQDWLNGKEYKCKVSNKDLPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 437 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEMLPLPSVFLFPPKPKDTLMISRTPEVTCVVVDVSHEDPEVKFNWYVDGVEVHNAKTKPREEQYNQRYRVVSVLTVLHQDWLNGKEYKCKVSNKDLPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 438 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEMLPLPSVFLFPPKPKDTLMISRTPEVTCVVVDVSHEDPEVKFNWYVDGVEVHNAKTKPREEQYNQTYRVVSVLTVLHQDWLNGKEYKCKVSDKDLPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 439 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVWDVSHEDPEVKFNWYVDGVEVHNAKTKPREEQYNSTYRVVSVLTVLHQDWLNGKEYKCKVSNKDLPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 440-449 - skipped (10 entries)

SEQ ID NO: 450 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVWDVSHEDPEVKFNWYVDGVEVHNAKTKPREEQYNSTYRVVSVLTVLHQDWLNGKEYKCKVSDKDLPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 451 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVWDHSHEDPEVKFNWYVDGVEVHNAKTKPREEQYNSTYRVVSVLTVLHQDWLNGKEYKCKVSDKDLPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 452 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSQYDPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 453 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDASQYDPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 454 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVVDVSQYDPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 455 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSNYDPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 456 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVVDVSQYEPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 457 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVVDASQYEPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 458 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVVDVSNYEPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 459 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVVFVSQYEPEVKFNWYVDGVEVHNAKTKPREEQNNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 460 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPMKEGAPSVFLFPPKPKDTLMISRTPEVTCVVVDVSPKSPEVEFNWYVDGVEVHNAKTKPREEQYNSTYRVVSVLTVLHQDWLNGKEYKCKVSTSALAAEIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 461 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPGPGSPSVFLFPPKPKDTLMISRTPEVTCVVVDVSPADPEVHFNWYVDGVEVHNAKTKPREEQYNSTYRVVSVLTVLHQDWLNGKEYKCKVSNNALIGQIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 462 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPVISGGPSVFLFPPKPKDTLMISRTPEVTCVVVDVSPENPEVKFNWYVDGVEVHNAKTKPREEQYNSTYRVVSVLTVLHQDWLNGKEYKCKVSTKNHPQPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 463 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPKLLGAPSVFLFPPKPKDTLMISRTPEVTCVVVDVSPSDPEVHFNWYVDGVEVHNAKTKPREEQYNSTYRVVSVLTVLHQDWLNGKEYKCKVSNKNVNGVIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 464 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 465 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVWDHSHEDPEVKFNWYVDGVEVHNAKTKPREEQYNSTYRVVSVLTVLHQDWLNGKEYKCKVSNKDLPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 466 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 467 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 468 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 469 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGNSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 470 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGENSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 471 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDNSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 472 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGNSVFLFPPKPKDTLMISRTPEVTCVVVDVKHEDPEVKFNWYVDGVEVHNAKTKPREEQYNSTYRVVSVLTVLHQDWLNGKEYKCKVSNKAMPEPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 473 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGFSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 474 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEFSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 475 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDFSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 476 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGFSVFLFPPKPKDTLMISRTPEVTCVVVDVKHEDPEVKFNWYVDGVEVHNAKTKPREEQYNSTYRVVSVLTVLHQDWLNGKEYKCKVSNKAMPEPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 477 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGQSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 478 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEQSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 479 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDQSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 480 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGQSVFLFPPKPKDTLMISRTPEVTCVVVDVKHEDPEVKFNWYVDGVEVHNAKTKPREEQYNSTYRVVSVLTVLHQDWLNGKEYKCKVSNKAMPEPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 481 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGMSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 482 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEMSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 483 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDMSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 484 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGMSVFLFPPKPKDTLMISRTPEVTCVVVDVKHEDPEVKFNWYVDGVEVHNAKTKPREEQYNSTYRVVSVLTVLHQDWLNGKEYKCKVSNKAMPEPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 485 - protein, 331 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEGLLGGDSVFLFPPKPKDTLMISRTPEVTCVVVDVKHEDPEVKFNWYVDGVEVHNAKTKPREEQYNSTYRVVSVLTVLHQDWLNGKEYKCKVSNKAMPEPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 486 - protein, 331 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEGLLGGDSVFLFPPKPKDTLMISRTPEVTCVVVDVKHEDPEVKFNWYVDGVEVHNAKTKPREEQYNSTYRVVSVLTVLHQDWLNGKEYKCKVSNKDMPEPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 487 - protein, 331 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEGLLGHDSVFLFPPKPKDTLMISRTPEVTCVVVDVKHEDPEVKFNWYVDGVEVHNAKTKPREEQYNSTYRVVSVLTVLHQDWLNGKEYKCKVSNKAMPEPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 488 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSQYDPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 489 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDASQYDPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 490 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVVDVSQYDPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 491 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSNYDPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 492 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVVDVSQYEPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 493 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVVDASQYEPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 494 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVVDVSNYEPEVKFNWYVDGVEVHNAKTKPREEQNNSFYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 495 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPEVFLFPPKPKDTLMISRTPEVTCVVVFVSQYEPEVKFNWYVDGVEVHNAKTKPREEQNNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 496 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALDAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 497 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKDLDAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 498 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSDKALDAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 499 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSDKDLDAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 500 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALEAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 501 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 502 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSDEDGEVKFNWYVDGVEVHNAKTKPREEQYNSTYRVVSVLTVLHQDWLNGKEYKCKVSNKALAAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 503 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHDEPEVKFNWYVDGVEVHNAKTKPREEQYNSTYRVVSVLTVLHQDWLNGKEYKCKVSNKALPRPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 504 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGESVFLFPPKPKDTLMISRTPEVTCVVVDVSHDEPEVKFNWYVDGVEVHNAKTKPREEQYNSTYRVVSVLTVLHQDWLNGKEYKCKVSNKALPRPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 505 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGSSVFLFPPKPKDTLMISRTPEVTCVVVDVSHDEPEVKFNWYVDGVEVHNAKTKPREEQYNSTYRVVSVLTVLHQDWLNGKEYKCKVSNKALPRPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 506 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSDEDGEVQFNWYVDGVEVHNAKTKPREEQFNSTYRVVSVLTVLHQDWLNGKEYKCKVSNKALASSIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 507 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDGSVFLFPPKPKDTLMISRTPEVTCVVVDVSHDEPEVKFNWYVDGVEVHNAKTKPREEQYNSTYRVVSVLTVLHQDWLNGKEYKCKVSNKALPRPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 508 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPDVFLFPPKPKDTLMISRTPEVTCVVVDVSDEDGEVQFNWYVDGVEVHNAKTKPREEQFNSTYRVVSVLTVLHQDWLNGKEYKCKVSNKALASSIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 509 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDSGVFLFPPKPKDTLMISRTPEVTCVVVDVSHDEPEVKFNWYVDGVEVHNAKTKPREEQYNSTYRVVSVLTVLHQDWLNGKEYKCKVSNKALPRPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 510 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKALPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 511 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDGSAFLFPPKPKDTLMISRTPEVTCVVVDVSHDEPEVKFNWYVDGVEVHNAKTKPREEQYNSTYRVVSVLTVLHQDWLNGKEYKCKVSNKALPRPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 512 - protein, 333 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEGEVLVGGDSVFLFPPKPKDTLMISRTPEVTCVVVDVSHDEPEVKFNWYVDGVEVHNAKTKPREEQYNSTYRVVSVLTVLHQDWLNGKEYKCKVSNKALPRPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 513 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 514 - protein, 119 aa, synthetic construct
QVQLVQSGAEVKKPGASVKVSCKVSGYSLSKYDMSWVRQAPGKGLEWMGIIYTSGYTDYAQKFQGRVTMTEDTSTDTAYMELSSLRSEDTAVYYCATGNPYYTNGFNSWGQGTLVTVSS

SEQ ID NO: 515 - protein, 110 aa, synthetic construct
DIQMTQSPSSLSASVGDRVTITCQASQSPNNLLAWYQQKPGKAPKLLIYGASDLPSGVPSRFSGSGSGTDFTLTISSLQPEDFATYYCQNNYYVGPVSYAFGGGTKVEIK

SEQ ID NO: 516 - protein, 330 aa, synthetic construct [sequence omitted]

SEQ ID NO: 517 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 518 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 519 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 520 - protein, 23 aa, synthetic construct
EPKSCDKTHTCPPCPAPELLGGP

SEQ ID NO: 521 - protein, 25 aa, synthetic construct
ELKTPLGDTTHTCPRCPAPELLGGP

SEQ ID NO: 522 - protein, 70 aa, synthetic construct
ELKTPLGDTTHTCPRCPEPKSCDTPPPCPRCPEPKSCDTPPPCPRCPEPKSCDTPPPCPRCPAPELLGGP

SEQ ID NO: 523 - protein, 20 aa, synthetic construct
ESKYGPPCPSCPAPEFLGGP

SEQ ID NO: 524 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 525 - protein, 109 aa, synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 526 - protein, 110 aa, synthetic construct
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 527 - protein, 110 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 528 - protein, 101 aa, synthetic construct
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKA

SEQ ID NO: 529 - protein, 101 aa, synthetic construct
SVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKA

SEQ ID NO: 530 - protein, 118 aa, synthetic construct
EVQLLESGGGLVQPGGSLRLSCAASGFTFSDYYMSWVRQAPGKGLEWVSVISNSGGSTYYTDSVKGRFTISRDNSKNTLYLQMNSLRAEDTAVYYCTKDIGMTYFDYWGQGTLVTVSS

SEQ ID NO: 531 - protein, 109 aa, synthetic construct
QSVLTQPPSASGAPGQRVTISCSGSYSNIGESYVSWYQQLPGTAPKLLIYLNSQRPSGVPDRFSGSKSGTSASLAISGLQSEDEADYYCAAWDDLNVWVFGGGTKLTVL

SEQ ID NO: 532 - protein, 109 aa, synthetic construct
QSVLTQPPSASGAPGQRVTISCSGSYSNIGGSYVSWYQQLPGTAPKLLIYLNSQRPSGVPDRFSGSKSGTSASLAISGLQSEDEADYYCAAWDDLNVWVFGGGTKLTVL

SEQ ID NO: 533 - protein, 109 aa, synthetic construct
QSVLTQPPSASGAPGQRVTISCSGSYSNIGLSYVSWYQQLPGTAPKLLIYLNSQRPSGVPDRFSGSKSGTSASLAISGLQSEDEADYYCAAWDDLNVWVFGGGTKLTVL

SEQ ID NO: 534 - protein, 109 aa, synthetic construct
QSVLTQPPSASGAPGQRVTISCSGSYSNIGQSYVSWYQQLPGTAPKLLIYLNSQRPSGVPDRFSGSKSGTSASLAISGLQSEDEADYYCAAWDDLNVWVFGGGTKLTVL

SEQ ID NO: 535 - protein, 109 aa, synthetic construct
QSVLTQPPSASGAPGQRVTISCSGSYSNIGSSYVSWYQQLPGTAPKLLIYLNSQRPSGVPDRFSGSKSGTSASLAISGLQSEDEADYYCAAWDDLNVWVFGGGTKLTVL

SEQ ID NO: 536 - protein, 109 aa, synthetic construct
QSVLTQPPSASGAPGQRVTISCSGSYSNIGNDYVSWYQQLPGTAPKLLIYLNSQRPSGVPDRFSGSKSGTSASLAISGLQSEDEADYYCAAWDDLNVWVFGGGTKLTVL

SEQ ID NO: 537 - protein, 109 aa, synthetic construct
QSVLTQPPSASGAPGQRVTISCSGSYSNIGNQYVSWYQQLPGTAPKLLIYLNSQRPSGVPDRFSGSKSGTSASLAISGLQSEDEADYYCAAWDDLNVWVFGGGTKLTVL

SEQ ID NO: 538 - protein, 120 aa, synthetic construct
QVTLKESGPTLVKPTQTLTLTCTFSGFSLSTFGMGVGWIRQPPGKALEWLAHIWWDDDKYYNPALKSRLTITKDTSKNQVVLTMTNMDPVDTATYYCARIITTAWYFDVWGTGTTVTVSS

SEQ ID NO: 539 - protein, 109 aa, synthetic construct
QAVVTQEPSLTVSPGGTVTLTCRSSTGAVTTSNYANWVQQKPGQAFRGLIGGTNNRAPGVPDRFSGSILGNKAALTITGAQADDESIYFCALWYSNHFIFGSGTKVTVL

SEQ ID NO: 540 - protein, 120 aa, synthetic construct
QVTLKESGPTLVKPTQTLTLTCSFSGFSLSTFGMGVGWIRQPPGKGLEWIGHIWWDDDKYYNPALKSRLTITKDTSKNQVVLTMTNMDPVDTATYYCARIITTAWYFDVWGTGTTVTVSS

SEQ ID NO: 541 - protein, 109 aa, synthetic construct
QAVVTQEPSLTVSPGGTVTLTCRSSTGAVTTSNYANWVQQKPGQAFRGLIGGTNNRAPGVPDRFSGSLIGDKAALTITGAQADDESDYYCALWYSNHFIFGSGTKVTVL

SEQ ID NO: 542 - protein, 109 aa, synthetic construct
QAVVTQEPSLTVSPGGTVTLTCRSSTGAVTTSNYANWVQQKPGQAFRGLIGGTNNRAPGVPDRFSGSILGNKAALTITGAQADDESDYYCALWYSNHFIFGSGTKVTVL

SEQ ID NO: 543 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 544 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDGSAFLFPPKPKDTLMISRTPEVTCVVVDVSHDEPEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 545 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSDEDGEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 546 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 547 - protein, 5 aa, synthetic construct
SDAMN

SEQ ID NO: 548 - protein, 16 aa, synthetic construct
TIYSGGSTYYADSVKG

SEQ ID NO: 549 - protein, 10 aa, synthetic construct
GGNFYNYFDY

SEQ ID NO: 550 - protein, 5 aa, synthetic construct
DHYMS

SEQ ID NO: 551 - protein, 17 aa, synthetic construct
TISSGGNYIYYADSVKG

SEQ ID NO: 552 - protein, 13 aa, synthetic construct
ILKNGKYIYYFDY

SEQ ID NO: 553 - protein, 5 aa, synthetic construct
DYYMS

SEQ ID NO: 554 - protein, 17 aa, synthetic construct
VISNSGGSTYYTDSVKG

SEQ ID NO: 555 - protein, 9 aa, synthetic construct
DIGMTYFDY

SEQ ID NO: 556 - protein, 7 aa, synthetic construct
TFGMGVG

SEQ ID NO: 557 - protein, 16 aa, synthetic construct
HIWWDDDKYYNPALKS

SEQ ID NO: 558 - protein, 10 aa, synthetic construct
IITTAWYFDV

SEQ ID NO: 559 - protein, 11 aa, synthetic construct
RASQSVSSNLA

SEQ ID NO: 560 - protein, 7 aa, synthetic construct
GASTRAT

SEQ ID NO: 561 - protein, 10 aa, synthetic construct [sequence omitted]

SEQ ID NO: 562 - protein, 13 aa, synthetic construct
SGSYSNIGNSYVS

SEQ ID NO: 563 - protein, 7 aa, synthetic construct
LNSQRPS

SEQ ID NO: 564 - protein, 10 aa, synthetic construct
AAWDDLNVWV

SEQ ID NO: 565 - protein, 14 aa, synthetic construct
RSSTGAVTTSNYAN

SEQ ID NO: 566 - protein, 7 aa, synthetic construct
GTNNRAP

SEQ ID NO: 567 - protein, 9 aa, synthetic construct
ALWYSNHFI

SEQ ID NO: 568 - protein, 13 aa, synthetic construct
SGSYSNIGESYVS

SEQ ID NO: 569 - protein, 13 aa, synthetic construct
SGSYSNIGGSYVS

SEQ ID NO: 570 - protein, 13 aa, synthetic construct
SGSYSNIGLSYVS

SEQ ID NO: 571 - protein, 13 aa, synthetic construct
SGSYSNIGQSYVS

SEQ ID NO: 572 - protein, 13 aa, synthetic construct
SGSYSNIGSSYVS

SEQ ID NO: 573 - protein, 13 aa, synthetic construct
SGSYSNIGNDYVS

SEQ ID NO: 574 - protein, 13 aa, synthetic construct
SGSYSNIGNQYVS

SEQ ID NO: 575 - protein, 5 aa, synthetic construct
DYHMS

SEQ ID NO: 576 - protein, 16 aa, synthetic construct
SIDTEGKTYYPDSVKG

SEQ ID NO: 577 - protein, 9 aa, synthetic construct
DVGDWYFDL

SEQ ID NO: 578 - protein, 5 aa, synthetic construct
SYGMH

SEQ ID NO: 579 - protein, 17 aa, synthetic construct
AISYDASTIDYADSVEG

SEQ ID NO: 580 - protein, 8 aa, synthetic construct
DGISGIDY

SEQ ID NO: 581 - protein, 16 aa, synthetic construct
RSSRSLVHGSGDNYLH

SEQ ID NO: 582 - protein, 7 aa, synthetic construct
MASNRAP

SEQ ID NO: 583 - protein, 9 aa, synthetic construct
MQALRAPFS

SEQ ID NO: 584 - protein, 11 aa, synthetic construct
RASQGIGSYLA

SEQ ID NO: 585 - protein, 7 aa, synthetic construct
EASRLES

SEQ ID NO: 586 - protein, 9 aa, synthetic construct
QQGYNAPIT

SEQ ID NO: 587 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 588 - protein, 19 aa, synthetic construct
MGWSCIILFLVATATGVHS

SEQ ID NO: 589 - protein, 288 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 590 - protein, 8 aa, synthetic construct
RFTFSDYH

SEQ ID NO: 591 - protein, 7 aa, synthetic construct
IDTEGKT

SEQ ID NO: 592 - protein, 11 aa, synthetic construct
ARDVGDWYFDL

SEQ ID NO: 593 - protein, 11 aa, synthetic construct
RSLVHGSGDNY

SEQ ID NO: 594 - skipped

SEQ ID NO: 595 - protein, 10 aa, synthetic construct
GVSLSNARMG

SEQ ID NO: 596 - protein, 7 aa, synthetic construct
VHTSGST

SEQ ID NO: 597 - protein, 10 aa, synthetic construct
ARDAGNWFDP

SEQ ID NO: 598 - protein, 7 aa, synthetic construct
QSLSSSH

SEQ ID NO: 599 - skipped

SEQ ID NO: 600 - protein, 9 aa, synthetic construct
QQYAVPPIT

SEQ ID NO: 601 - protein, 8 aa, synthetic construct
GFNFGAFA

SEQ ID NO: 602 - protein, 8 aa, synthetic construct
INQGGSEV

SEQ ID NO: 603 - protein, 13 aa, synthetic construct
ARDPGLPVSAFDI

SEQ ID NO: 604 - protein, 6 aa, synthetic construct
QDISNW

SEQ ID NO: 605 - protein, 9 aa, synthetic construct
HQIYDTPPT

SEQ ID NO: 606 - protein, 8 aa, synthetic construct
GSTLSSYG

SEQ ID NO: 607 - protein, 8 aa, synthetic construct
ISYDASTI

SEQ ID NO: 608 - protein, 10 aa, synthetic construct
ASEYGLAFDQ

SEQ ID NO: 609 - protein, 6 aa, synthetic construct
QGIGSY

SEQ ID NO: 610 - skipped

SEQ ID NO: 611 - protein, 446 aa, synthetic construct
EVQLVESGGGLVQPGGSLRLSCAASRFTFSDYHMSWVRQAPGKGLELVASIDTEGKTYYPDSVKGRFTISRDNAKNSLYLQMNSLRAEDTAVYYCARDVGDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 612 - protein, 446 aa, synthetic construct
EVQLVESGGGLVQPGGSLRLSCAASRFTFSDYHMSWVRQAPGKGLELVASIDTEGKTYYPDSVKGRFTISRDNAKNSLYLQMNSLRAEDTAVYYCARDVGDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 613 - protein, 446 aa, synthetic construct
EVQLVESGGGLVQPGGSLRLSCAASRFTFSDYHMSWVRQAPGKGLELVASIDTEGKTYYPDSVKGRFTISRDNAKNSLYLQMNSLRAEDTAVYYCARDVGDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 614 - protein, 446 aa, synthetic construct
EVQLVESGGGLVQPGGSLRLSCAASRFTFSDYHMSWVRQAPGKGLELVASIDTEGKTYYPDSVKGRFTISRDNAKNSLYLQMNSLRAEDTAVYYCARDVGDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 615 - protein, 446 aa, synthetic construct
EVQLVESGGGLVQPGGSLRLSCAASRFTFSDYHMSWVRQAPGKGLELVASIDTEGKTYYPDSVKGRFTISRDNAKNSLYLQMNSLRAEDTAVYYCARDVGDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 616 - protein, 446 aa, synthetic construct
EVQLVESGGGLVQPGGSLRLSCAASRFTFSDYHMSWVRQAPGKGLELVASIDTEGKTYYPDSVKGRFTISRDNAKNSLYLQMNSLRAEDTAVYYCARDVGDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 617 - protein, 446 aa, synthetic construct
EVQLVESGGGLVQPGGSLRLSCAASRFTFSDYHMSWVRQAPGKGLELVASIDTEGKTYYPDSVKGRFTISRDNAKNSLYLQMNSLRAEDTAVYYCARDVGDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 618 - protein, 446 aa, synthetic construct
EVQLVESGGGLVQPGGSLRLSCAASRFTFSDYHMSWVRQAPGKGLELVASIDTEGKTYYPDSVKGRFTISRDNAKNSLYLQMNSLRAEDTAVYYCARDVGDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 619 - protein, 446 aa, synthetic construct
EVQLVESGGGLVQPGGSLRLSCAASRFTFSDYHMSWVRQAPGKGLELVASIDTEGKTYYPDSVKGRFTISRDNAKNSLYLQMNSLRAEDTAVYYCARDVGDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 620 - protein, 447 aa, synthetic construct
QVQLQESGPGLVKPSQTLSLTCTVSGVSLSNARMGWSWIRQPPGKGLEWIGYVHTSGSTDYNPSLKSRVTMSVDTSKNQFSLKVNSVTAADTAVYYCARDAGNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 621 - protein, 447 aa, synthetic construct
QVQLQESGPGLVKPSQTLSLTCTVSGVSLSNARMGWSWIRQPPGKGLEWIGYVHTSGSTDYNPSLKSRVTMSVDTSKNQFSLKVNSVTAADTAVYYCARDAGNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 622 - protein, 447 aa, synthetic construct
QVQLQESGPGLVKPSQTLSLTCTVSGVSLSNARMGWSWIRQPPGKGLEWIGYVHTSGSTDYNPSLKSRVTMSVDTSKNQFSLKVNSVTAADTAVYYCARDAGNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 623 - protein, 447 aa, synthetic construct
QVQLQESGPGLVKPSQTLSLTCTVSGVSLSNARMGWSWIRQPPGKGLEWIGYVHTSGSTDYNPSLKSRVTMSVDTSKNQFSLKVNSVTAADTAVYYCARDAGNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 624 - protein, 447 aa, synthetic construct
QVQLQESGPGLVKPSQTLSLTCTVSGVSLSNARMGWSWIRQPPGKGLEWIGYVHTSGSTDYNPSLKSRVTMSVDTSKNQFSLKVNSVTAADTAVYYCARDAGNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 625 - protein, 447 aa, synthetic construct
QVQLQESGPGLVKPSQTLSLTCTVSGVSLSNARMGWSWIRQPPGKGLEWIGYVHTSGSTDYNPSLKSRVTMSVDTSKNQFSLKVNSVTAADTAVYYCARDAGNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 626 - protein, 447 aa, synthetic construct
QVQLQESGPGLVKPSQTLSLTCTVSGVSLSNARMGWSWIRQPPGKGLEWIGYVHTSGSTDYNPSLKSRVTMSVDTSKNQFSLKVNSVTAADTAVYYCARDAGNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 627 - protein, 447 aa, synthetic construct
QVQLQESGPGLVKPSQTLSLTCTVSGVSLSNARMGWSWIRQPPGKGLEWIGYVHTSGSTDYNPSLKSRVTMSVDTSKNQFSLKVNSVTAADTAVYYCARDAGNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 628 - protein, 447 aa, synthetic construct
QVQLQESGPGLVKPSQTLSLTCTVSGVSLSNARMGWSWIRQPPGKGLEWIGYVHTSGSTDYNPSLKSRVTMSVDTSKNQFSLKVNSVTAADTAVYYCARDAGNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 629 - protein, 449 aa, synthetic construct
EVQLVESGGGLVQPGRSLRLSCAASGFNFGAFAMHWVRQAPGKGLEWVSAINQGGSEVDYADSVEGRFTISRDNAKNSLYLQMNSLRAEDTAVYYCARDPGLPVSAFDIWGLGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 630 - protein, 449 aa, synthetic construct
EVQLVESGGGLVQPGRSLRLSCAASGFNFGAFAMHWVRQAPGKGLEWVSAINQGGSEVDYADSVEGRFTISRDNAKNSLYLQMNSLRAEDTAVYYCARDPGLPVSAFDIWGLGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 631 - protein, 449 aa, synthetic construct
EVQLVESGGGLVQPGRSLRLSCAASGFNFGAFAMHWVRQAPGKGLEWVSAINQGGSEVDYADSVEGRFTISRDNAKNSLYLQMNSLRAEDTAVYYCARDPGLPVSAFDIWGLGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 632 - protein, 449 aa, synthetic construct
EVQLVESGGGLVQPGRSLRLSCAASGFNFGAFAMHWVRQAPGKGLEWVSAINQGGSEVDYADSVEGRFTISRDNAKNSLYLQMNSLRAEDTAVYYCARDPGLPVSAFDIWGLGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 633 - protein, 449 aa, synthetic construct
EVQLVESGGGLVQPGRSLRLSCAASGFNFGAFAMHWVRQAPGKGLEWVSAINQGGSEVDYADSVEGRFTISRDNAKNSLYLQMNSLRAEDTAVYYCARDPGLPVSAFDIWGLGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 634 - protein, 449 aa, synthetic construct
EVQLVESGGGLVQPGRSLRLSCAASGFNFGAFAMHWVRQAPGKGLEWVSAINQGGSEVDYADSVEGRFTISRDNAKNSLYLQMNSLRAEDTAVYYCARDPGLPVSAFDIWGLGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 635 - protein, 449 aa, synthetic construct
EVQLVESGGGLVQPGRSLRLSCAASGFNFGAFAMHWVRQAPGKGLEWVSAINQGGSEVDYADSVEGRFTISRDNAKNSLYLQMNSLRAEDTAVYYCARDPGLPVSAFDIWGLGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 636 - protein, 449 aa, synthetic construct
EVQLVESGGGLVQPGRSLRLSCAASGFNFGAFAMHWVRQAPGKGLEWVSAINQGGSEVDYADSVEGRFTISRDNAKNSLYLQMNSLRAEDTAVYYCARDPGLPVSAFDIWGLGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 637 - protein, 449 aa, synthetic construct
EVQLVESGGGLVQPGRSLRLSCAASGFNFGAFAMHWVRQAPGKGLEWVSAINQGGSEVDYADSVEGRFTISRDNAKNSLYLQMNSLRAEDTAVYYCARDPGLPVSAFDIWGLGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 638 - protein, 446 aa, synthetic construct
EVQLVESGGGLVQPGRSLRLSCAASGSTLSSYGMHWVRQAPGKGLEWVSAISYDASTIDYADSVEGRFTISRDNAKNSLYLQMNSLRAEDTAVYYCASEYGLAFDQWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 639 - protein, 446 aa, synthetic construct
EVQLVESGGGLVQPGRSLRLSCAASGSTLSSYGMHWVRQAPGKGLEWVSAISYDASTIDYADSVEGRFTISRDNAKNSLYLQMNSLRAEDTAVYYCASEYGLAFDQWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 640 - protein, 446 aa, synthetic construct
EVQLVESGGGLVQPGRSLRLSCAASGSTLSSYGMHWVRQAPGKGLEWVSAISYDASTIDYADSVEGRFTISRDNAKNSLYLQMNSLRAEDTAVYYCASEYGLAFDQWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 641 - protein, 446 aa, synthetic construct
EVQLVESGGGLVQPGRSLRLSCAASGSTLSSYGMHWVRQAPGKGLEWVSAISYDASTIDYADSVEGRFTISRDNAKNSLYLQMNSLRAEDTAVYYCASEYGLAFDQWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 642 - protein, 446 aa, synthetic construct
EVQLVESGGGLVQPGRSLRLSCAASGSTLSSYGMHWVRQAPGKGLEWVSAISYDASTIDYADSVEGRFTISRDNAKNSLYLQMNSLRAEDTAVYYCASEYGLAFDQWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 643 - protein, 446 aa, synthetic construct
EVQLVESGGGLVQPGRSLRLSCAASGSTLSSYGMHWVRQAPGKGLEWVSAISYDASTIDYADSVEGRFTISRDNAKNSLYLQMNSLRAEDTAVYYCASEYGLAFDQWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 644 - protein, 446 aa, synthetic construct
EVQLVESGGGLVQPGRSLRLSCAASGSTLSSYGMHWVRQAPGKGLEWVSAISYDASTIDYADSVEGRFTISRDNAKNSLYLQMNSLRAEDTAVYYCASEYGLAFDQWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 645 - protein, 446 aa, synthetic construct
EVQLVESGGGLVQPGRSLRLSCAASGSTLSSYGMHWVRQAPGKGLEWVSAISYDASTIDYADSVEGRFTISRDNAKNSLYLQMNSLRAEDTAVYYCASEYGLAFDQWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 646 - protein, 446 aa, synthetic construct
EVQLVESGGGLVQPGRSLRLSCAASGSTLSSYGMHWVRQAPGKGLEWVSAISYDASTIDYADSVEGRFTISRDNAKNSLYLQMNSLRAEDTAVYYCASEYGLAFDQWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 647 - protein, 219 aa, synthetic construct
DIVMTQSPLSLPVTPGEPASISCRSSRSLVHGSGDNYLHWYLQKPGQSPQLLIYMASNRAPGVPDRFSGSGSGTDFTLKISRVEAEDVGVYYCMQALRAPFSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 648 - protein, 219 aa, synthetic construct; identical to SEQ ID NO: 647

SEQ ID NO: 649 - protein, 219 aa, synthetic construct; identical to SEQ ID NO: 647

SEQ ID NO: 650 - protein, 219 aa, synthetic construct; identical to SEQ ID NO: 647

SEQ ID NO: 651 - protein, 219 aa, synthetic construct; identical to SEQ ID NO: 647

SEQ ID NO: 652 - protein, 219 aa, synthetic construct; identical to SEQ ID NO: 647

SEQ ID NO: 653 - protein, 219 aa, synthetic construct; identical to SEQ ID NO: 647

SEQ ID NO: 654 - protein, 219 aa, synthetic construct; identical to SEQ ID NO: 647

SEQ ID NO: 655 - protein, 219 aa, synthetic construct; identical to SEQ ID NO: 647

SEQ ID NO: 656 - protein, 215 aa, synthetic construct
EIVMTQSPATLSLSPGERATLSCRASQSLSSSHLAWYQQKPGQAPRLLIYDASIRVPGIPARFSGSGSGTDFTLTISSLEPEDFAVYYCQQYAVPPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 657 - protein, 215 aa, synthetic construct; identical to SEQ ID NO: 656

SEQ ID NO: 658 - protein, 215 aa, synthetic construct; identical to SEQ ID NO: 656

SEQ ID NO: 659 - protein, 215 aa, synthetic construct; identical to SEQ ID NO: 656

SEQ ID NO: 660 - protein, 215 aa, synthetic construct; identical to SEQ ID NO: 656

SEQ ID NO: 661 - protein, 215 aa, synthetic construct; identical to SEQ ID NO: 656

SEQ ID NO: 662 - protein, 215 aa, synthetic construct; identical to SEQ ID NO: 656

SEQ ID NO: 663 - protein, 215 aa, synthetic construct; identical to SEQ ID NO: 656

SEQ ID NO: 664 - protein, 215 aa, synthetic construct; identical to SEQ ID NO: 656

SEQ ID NO: 665 - protein, 214 aa, synthetic construct
DIQMTQSPSSLSASVGDRVTITCRASQDISNWLAWYQQKPGKAPKLLIYDASSLVSGVPSRFSGSGSGTDFTLTISSLQPEDVATYYCHQIYDTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 666 - protein, 214 aa, synthetic construct; identical to SEQ ID NO: 665

SEQ ID NO: 667 - protein, 214 aa, synthetic construct; identical to SEQ ID NO: 665

SEQ ID NO: 668 - protein, 214 aa, synthetic construct; identical to SEQ ID NO: 665

SEQ ID NO: 669 - protein, 214 aa, synthetic construct; identical to SEQ ID NO: 665

SEQ ID NO: 670 - protein, 214 aa, synthetic construct; identical to SEQ ID NO: 665

SEQ ID NO: 671 - protein, 214 aa, synthetic construct; identical to SEQ ID NO: 665

SEQ ID NO: 672 - protein, 214 aa, synthetic construct; identical to SEQ ID NO: 665

SEQ ID NO: 673 - protein, 214 aa, synthetic construct; identical to SEQ ID NO: 665

SEQ ID NO: 674 - protein, 214 aa, synthetic construct
DIQMTQSPSSLSASVGDRVTITCRASQGIGSYLAWYQQKPGKAPKLLIYEASRLESGVPSRFSGSGSGTDFTLTISSLQPEDVATYYCQQGYNAPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 675 - protein, 214 aa, synthetic construct; identical to SEQ ID NO: 674

SEQ ID NO: 676 - protein, 214 aa, synthetic construct; identical to SEQ ID NO: 674

SEQ ID NO: 677 - protein, 214 aa, synthetic construct; identical to SEQ ID NO: 674

SEQ ID NO: 678 - protein, 214 aa, synthetic construct; identical to SEQ ID NO: 674

SEQ ID NO: 679 - protein, 214 aa, synthetic construct; identical to SEQ ID NO: 674

SEQ ID NO: 680 - protein, 214 aa, synthetic construct; identical to SEQ ID NO: 674

SEQ ID NO: 681 - protein, 214 aa, synthetic construct; identical to SEQ ID NO: 674

SEQ ID NO: 682 - protein, 214 aa, synthetic construct; identical to SEQ ID NO: 674

SEQ ID NO: 683 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 684 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 685 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDGSAFLFPPKPKDTLMISRTPEVTCVVVDVSHDEPEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 686 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDGSAFLFPPKPKDTLYITREPEVTCVVVDVSHDEPEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 687 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSDEDGEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 688 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLYITREPEVTCVVVDVSDEDGEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 689 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 690 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 691 - protein, 447 aa, synthetic construct
EVQLLESGGGLVQPGGSLRLSCAASGFTFSDYYMSWVRQAPGKGLEWVSVISNSGGSTYYTDSVKGRFTISRDNSKNTLYLQMNSLRAEDTAVYYCTKDIGMTYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGEESVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 692 - protein, 215 aa, synthetic construct
QSVLTQPPSASGAPGQRVTISCSGSYSNIGNQYVSWYQQLPGTAPKLLIYLNSQRPSGVPDRFSGSKSGTSASLAISGLQSEDEADYYCAAWDDLNVWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS